(19) United States
(12) Patent Application Publication
(10) Pub. No.: US 2026/0071223 A1
(43) Pub. Date: Mar. 12, 2026

(54) LIPID CONJUGATES FOR THE DELIVERY OF THERAPEUTIC AGENTS TO ADIPOSE TISSUE

(71) Applicant: Arrowhead Pharmaceuticals, Inc., Pasadena, CA (US)

(72) Inventors: Mark Majewski, Rockton, IL (US); Agnieszka Glebocka, Madison, WI (US); Tao Pei, Middleton, WI (US); Jonathan Benson, Stoughton, WI (US); Zhao Xu, San Diego, CA (US)

(21) Appl. No.: 19/259,183

(22) Filed: Jul. 3, 2025

Related U.S. Application Data
(63) Continuation of application No. PCT/US24/10584, filed on Jan. 5, 2024.
(60) Provisional application No. 63/478,795, filed on Jan. 6, 2023.
(60) Provisional application No. 63/612,901, filed on Dec. 20, 2023.

(51) Int. Cl. C12N 15/113

(52) U.S. Cl. CPC C12N 15/1138; C12N 2310/14; C12N 2310/3515

(57) ABSTRACT

Disclosed herein are compounds comprising lipid PK/PD modulators for delivery of oligonucleotide-based agents, e.g., double stranded RNAi agents or antisense oligonucleotides, to certain tissues (e.g., adipose tissue) or cell types (e.g., adipocytes) in vivo. The PK/PD modulators disclosed herein, when conjugated to an oligonucleotide-based therapeutic or diagnostic agent, such as an RNAi agent, can enhance the delivery of the composition to the adipocytes in vivo to facilitate the inhibition of gene expression in those cells.

### RELATED APPLICATIONS

This application is a continuation application of International Application No. PCT/US2024/10584, filed on Jan. 5, 2024, which claims the benefit of priority of U.S. Provisional Patent Application Ser. No. 63/478,795, filed on Jan. 6, 2023, and 63/612,901, filed Dec. 20, 2023, the contents of each of which are incorporated herein by reference in their entirety.

### SEQUENCE LISTING

This application contains a Sequence Listing which has been submitted in XML format and is hereby incorporated by reference in its entirety. The XML copy is named 30714-US1_SeqListing.xml, created Jul. 2, 2025, and is 789,164 bytes in size.

### FIELD OF THE INVENTION

The present disclosure relates to lipid conjugates for the delivery of oligonucleotide-based agents (for example, double-stranded RNAi agents such as small interfering RNA (siRNA) and antisense oligonucleotides) to adipose tissue and/or certain adipose cell types (e.g., adipocytes) in vivo.

### BACKGROUND

Oligonucleotide-based agents, such double-stranded RNA interference (RNAi) agents and antisense oligonucleotides, have shown great promise and have the potential to revolutionize the field of medicine by providing patients with therapeutic treatment options previously unavailable. However, the effective in vivo delivery of oligonucleotide-based agents to the desired cells and tissues of interest, and double-stranded therapeutic RNAi agents in particular, has long been a challenge in developing viable therapeutics. While consistent delivery to hepatocyte cells in the liver using carbohydrates such as N-Acetyl-galactosamine has been well established in the field, significant challenges remain when trying to achieve specific and selective delivery of oligonucleotide-based agents to extra-hepatic cells.

While various attempts over the past several years have been made to direct oligonucleotide-based agents to certain extra-hepatic cell types, including cells in the central nervous system (CNS), adipocytes, cardiac myocytes, and others, using, for example, cholesterol conjugates (which is non-specific thereby having the disadvantage of distributing to various undesired tissues and organs) and lipid-nanoparticles (LNPs) (which similarly have the disadvantage of being non-specific and have frequently been reported to have toxicity concerns), to date none have achieved suitable delivery. Accordingly, there remains a need for a delivery vehicles to direct oligonucleotide-based agents, and RNAi agents in particular, to non-hepatocyte cell types. And with obesity becoming a serious public health concern with increasing incidence in adults and children, more than ever there is a need to deliver therapeutic agents, such as oligonucleotide-based agents, selectively and effectively to adipose tissue.

### SUMMARY OF THE INVENTION

Disclosed herein are compounds (e.g., compound of Formula (I)) comprising a lipid conjugated (or linked) to an oligonucleotide-based agent for delivery to adipose tissue or certain cell types (e.g., adipocytes). The lipids (also referred to herein as Lipid PK/PD modulators) when conjugated facilitate the delivery of the oligonucleotide-based agent payload to certain cell types (e.g., adipocytes) or to adipose tissue. Lipid PK/PD modulator precursors (e.g., compounds of Formula (II)) are also disclosed herein.

One aspect of this disclosure provides for double-stranded oligonucleotides wherein a lipid is conjugated to one of the terminal nucleotides of one of the strands. In some embodiments, the lipid is conjugated to the 5′ terminal nucleotide of one of the strands. In some embodiments, the lipid is conjugated to the 3′ terminal nucleotide of one of the strands. In some embodiments, a lipid is conjugated to both the 5′ terminal nucleotide and the 3′ terminal nucleotide of one of the strands. In some embodiments the lipid is conjugated internally (e.g., conjugated at the 2′ position) to one or more nucleotides on one or both of the strands.

In another aspect, disclosed herein are compounds of Formula (I)

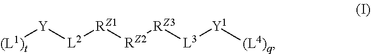

and pharmaceutically acceptable salts thereof, wherein R^{Z2} comprises an oligonucleotide containing about 8 to about 50 independently modified or unmodified nucleotides; L¹ and L⁴ are each independently a lipid comprising from about 10 to about 50 carbon atoms; and R^{Z1}, R^{Z3}, Y, Y¹, L², L³, t, and q are as defined herein. In some embodiments the lipid (e.g., L¹ and/or L⁴) conjugated to the oligonucleotide-based agent is saturated. In some embodiments, the lipid is unsaturated. In some embodiments, the lipid is a sterol. In some embodiments, the lipid is a saturated lipid having between 12 and 30 carbon atoms. In some embodiments, the lipid is a straight chain lipid having 16 carbon atoms. In some embodiments, the lipid contains a hydroxyl moiety. In some embodiments, the lipid contains a carboxylic acid moiety.

Further provided herein are methods of treating an adipose-related disorder (e.g., metabolic diseases such as obesity, type 2 diabetes mellitus, insulin resistance, metabolic syndrome, various lipodystrophies, lipedema atherosclerotic vascular disease, cardiometabolic diseases or disorders, or other similar diseases or disorders) in a subject in need thereof, comprising administering to the subject a therapeutically effective amount of any of the compounds or compositions described herein.

Unless otherwise defined, all technical and scientific terms used herein have the same meaning as commonly understood by one of ordinary skill in the art. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other objects, features, aspects, and advantages of the invention will be apparent from the following detailed description, accompanying figures, and from the claims.

### DETAILED DESCRIPTION

### Lipid PK/PD Modulators

Described herein are compounds (e.g., compounds of Formula (I)) comprising lipid PK/PD modulators conjugated to oligonucleotide-based agent(s) to provide delivery of payloads, such as RNA interference (RNAi) agents, to cells in vivo. Without being bound to any particular theory, it is believed that the compounds described herein modulate the pharmacokinetic and/or pharmacodynamic (PK/PD) properties of the RNAi agent to increase the delivery to adipose tissues and cells, thereby resulting in an increase in efficacy of the oligonucleotide-based therapeutic. The compounds described herein may facilitate delivery to certain cell types, including but not limited to adipose cell types, including white adipocytes and brown adipocytes.

Also described herein are compounds of Formula (I):

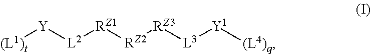

or a pharmaceutically acceptable salt thereof, wherein:
R^{Z1} and R^{Z3} each independently comprise a bond or a capping residue;
R^{Z2} comprises an oligonucleotide containing about 8 to about 50 nucleotides, each of which may be independently modified or unmodified;
Y is a bond or a linker connecting at least one L¹ to L² when present, or to R^{Z1};
Y¹ is a bond or a linker connecting at least one L³ to L⁴ when present, or to R^{Z3};
L² and L³ are independently absent, or a linker comprising 1-20 PEG units;
q is 1, 2, or 3, as valency permits;
t is 1, 2, or 3, as valency permits; and
L¹ and L⁴ are each independently a lipid comprising from about 10 to about 50 carbon atoms, or a capping moiety.

The compounds of Formula (I) contain the substituents R^{Z1} and R^{Z3}. In certain embodiments, R^{Z1} is an inverted abasic residue (e.g., R^{Z1} is (invAb)s as defined in Table 4). In certain embodiments, R^{Z3} is an inverted abasic residue (e.g., R^{Z3} is (invAb) as defined in Table 4). In certain embodiments, R^{Z1} is (invAb)s, and R^{Z3} is (invAb). In certain embodiments, R^{Z2} is conjugated to a 5′ carbon atom of a modified or unmodified ribose molecule of R^{Z2}. In certain embodiments, R^{Z1} is a bond. In certain embodiments, R^{Z3} is a bond.

The compounds of Formula (I) contain the substituent R^{Z2}. In certain embodiments, R^{Z2} is a single-stranded oligonucleotide. In certain embodiments, R^{Z2} is a double-stranded oligonucleotide. In certain embodiments, R^{Z2} comprises an oligonucleotide wherein the oligonucleotide comprises an antisense strand that is at least 70%, 80%, or 90% complementary to the mRNA of a gene expressed in adipose tissue (e.g., human adipose tissue). In certain embodiments, R^{Z2} comprises an oligonucleotide wherein the oligonucleotide comprises an antisense strand that is at least 70%, 80%, or 90% complementary to the mRNA of a gene expressed in an adipocyte (e.g., a human adipocyte). In certain embodiments, the gene is expressed in a mature adipocyte. In certain embodiments, the gene is expressed in a white adipocyte.

In some embodiments, the compounds of Formula (I) contain the substituent Y. In certain embodiments, Y is a bond. In certain embodiments, Y is a linker connecting at least one L¹ to L² if present, or L¹ to R^{Z1}. In certain embodiments, Y is a bivalent moiety connecting at least two L¹ substituents to L² if present, or connecting at least two L¹ substituents to R^{Z1}. In certain embodiments, Y is of the formula:

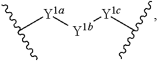

wherein Y^{a} and Y^{c} are each independently absent, —N(H)—, or —C(O)—; Y^{b} is substituted or unsubstituted heteroalkylene, substituted or unsubstituted carbocyclene, substituted or unsubstituted heterocyclene; substituted or unsubstituted arylene, or substituted or unsubstituted heteroarylene. In certain embodiments, Y^{a} is —C(O)—. In certain embodiments, Y^{a} is —N(H)—. In certain embodiments, Y^{c} is —C(O)—. In certain embodiments, Y^{c} is —N(H)—. In certain embodiments, Y^{b} is substituted or unsubstituted C₁-C₆ heteroalkylene. In certain embodiments, Y^{b} is of the formula

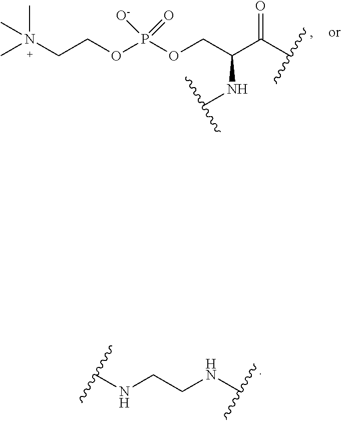

In certain embodiments, Y^{b} is absent. In certain embodiments, Y^{b} substituted or unsubstituted carbocyclene. In certain embodiments, Y^{b} is of the formula

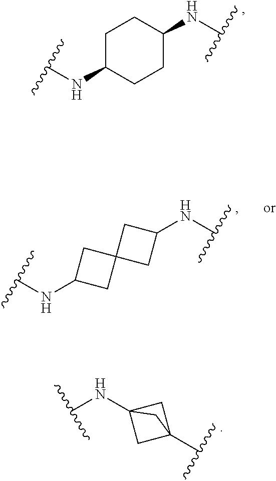 [SMILES: *N[C@H]1CC[C@@H](N*)CC1 ; *NC1CC2(C1)CC(N*)C2 ; *NC12CC(*)(C1)C2]

In certain embodiments, Y^{b} substituted or unsubstituted heterocyclene. In embodiments, Y^{b} is of the formula

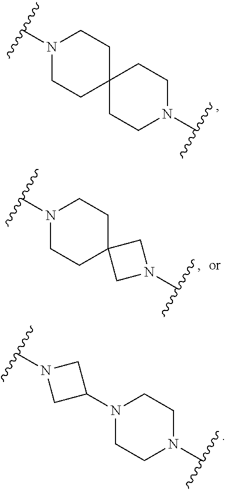 [SMILES: *N1CCC2(CC1)CCN(*)CC2 ; *N1CCC2(CC1)CN(*)C2 ; *N1CCN(C2CN(*)C2)CC1]

In certain embodiments, Y^{b} substituted or unsubstituted arylene. In certain embodiments, Y^{b} substituted or unsubstituted phenylene. In certain embodiments, Y^{b} is of the formula

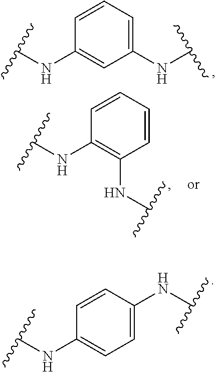

In certain embodiments, Y is selected from the group consisting of —N(H)—C(O)—, —C(O)—N(H)—,

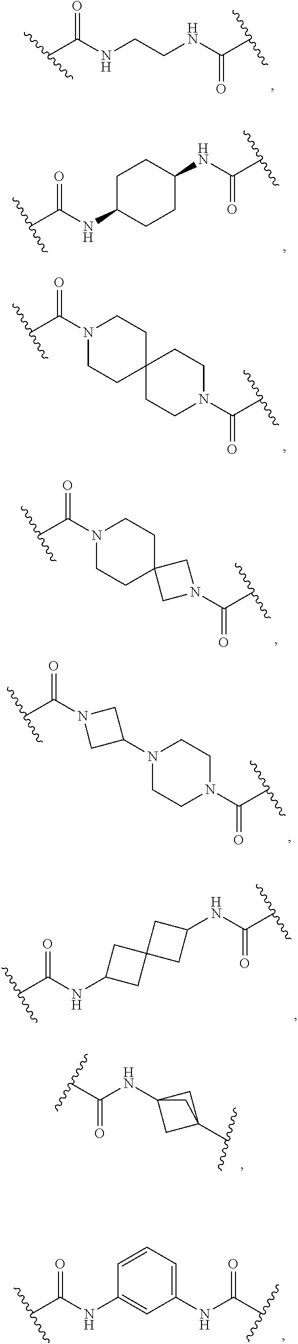

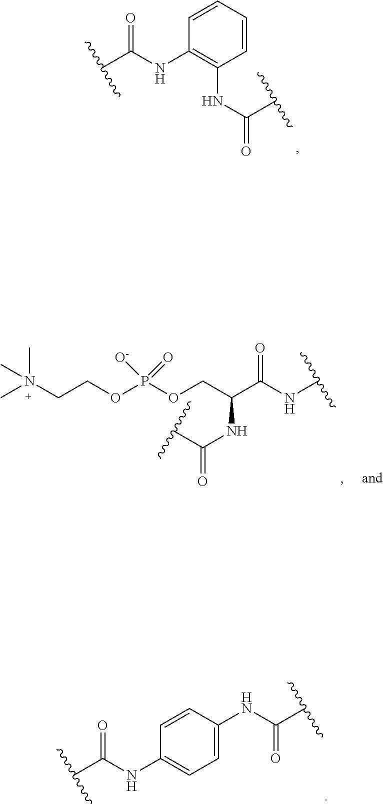

In certain embodiments, Y is a trivalent moiety connecting two L¹ groups to L², or connecting two L¹ groups to R^{Z1}. In certain embodiments, Y is of the formula:

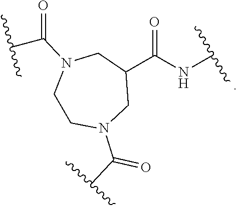 [SMILES: *NC(=O)C1CN(C(*)=O)CCN(C(*)=O)C1]

In some embodiments, Formula (I) contains the substituent L². In certain embodiments, L² is of the formula

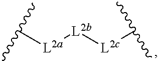

wherein:
L^{2a} is a bond or of the formula:

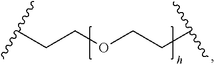

wherein h is an integer between 1 and 12;
L^{2b} is a bond or a chemical moiety formed by reacting a first reactive moiety with a second moiety; and
L^{2c} is a bond or a bidentate linking group.

In certain embodiments, L^{2a} is a bond. In certain embodiments, L^{2a} is of the formula:

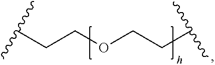

wherein h is an integer between 1 and 12. In certain embodiments, h is 2. In certain embodiments, h is 3. In certain embodiments, h is 5. In certain embodiments, h is 9. In certain embodiments, h is 10.

In certain embodiments, L^{2b} is a bond. In certain embodiments, L^{2b} is —C(O)—. In certain embodiments, L^{2b} is of the formula:

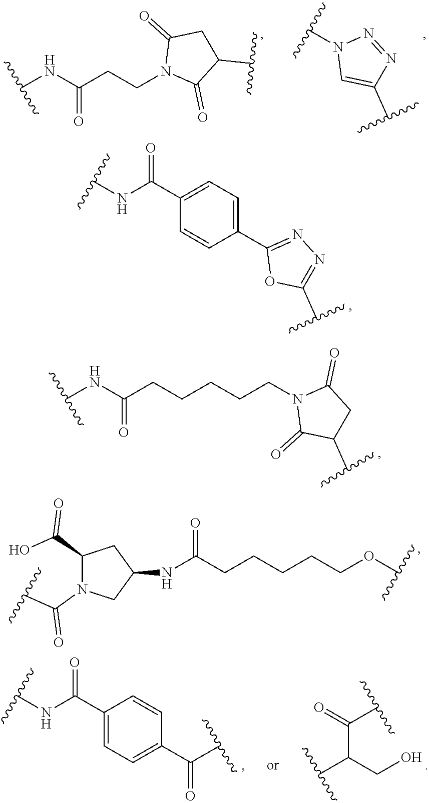

In certain embodiments, L^{2c} is of the formula:

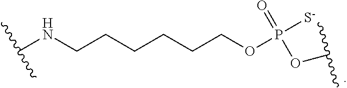 [SMILES: *NCCCCCCOP(=O)([S-])O*]

In certain embodiments, L^{2c} is of the formula:

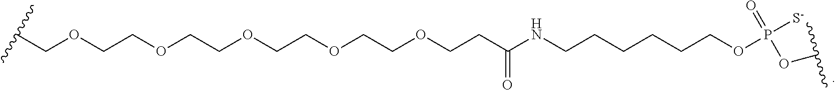 [SMILES: *COCCOCCOCCOCCOCCC(=O)NCCCCCCOP(=O)([S-])O*]

The compounds of Formula (I) contain t instances of the substituent L¹ and q instances of the substituent L⁴. In certain embodiments, L¹ and L⁴ are each independently a lipid comprising from about 10 to about 50 carbon atoms.

In certain embodiments, L¹ is a fatty acid, fatty acid derived group, glycerolipid, glycerolipid derived group, phospholipid, phospholipid derived group, sphingolipid, sphingolipid derived group, cholesterol ester, or a cholesterol ester derived group. In certain embodiments, at least one instance of L¹ is independently a straight chain lipid. In certain embodiments, at least one instance of L¹ is independently a saturated lipid. In certain embodiments, at least one instance of L¹ is independently an unsaturated lipid. In certain embodiments, at least one instance of L¹ is independently a branched lipid. In certain embodiments, L¹ is an NEM (i.e., N-ethylmaleimide) capping moiety.

In certain embodiments, at least one instance of L¹ is independently of the formula:

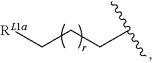

wherein R^{L1a} is H, or CO₂H; and r is an integer between 5 and 35. In certain embodiments, R^{L1a} is H. In certain embodiments, R^{L1a} is CO₂H. In certain embodiments, r is 8, 9, 10, 11, 12, 13, 14, 15, 16, 17, 18, 19, or 20. In certain embodiments, r is 9. In certain embodiments, r is 11. In certain embodiments, r is 12. In certain embodiments, r is 13. In certain embodiments, r is 14. In certain embodiments, r is 15. In certain embodiments, r is 17.

In certain embodiments, at least one instance of L¹ is independently of the formula

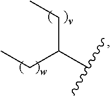

wherein w is an integer between 2 and 25; and v is an integer between 2 and 25. In certain embodiments, at least one instance of L¹ is independently of the formula:

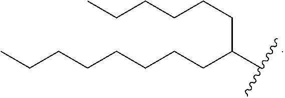 [SMILES: *C(CCCCCC)CCCCCCCC]

In certain embodiments, L¹ is independently a straight chain lipid comprising 1-8 alkenylene moieties. In certain embodiments, at least one instance of L¹ is independently of the formula:

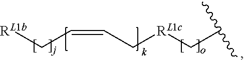

wherein R^{L1b} is —CH₃, or —CO₂H; R^{L1e} is —CH₂— or —C(O)—; j is an integer between 0 and 20; k is an integer between 1 and 8; and o is an integer between 1 and 20. In certain embodiments, R^{L1b} is —H. In certain embodiments, R^{L1b} is —CO₂H. In certain embodiments, R^{L1e} is —CH₂—. In certain embodiments, R^{L1b} is —C(O)—. In certain embodiments, j is 0. In certain embodiments, j is 1. In certain embodiments, j is 4. In certain embodiments, j is 7. In certain embodiments, k is 1. In certain embodiments, k is 2. In certain embodiments, k is 3. In certain embodiments, k is 4. In certain embodiments, k is 5. In certain embodiments, o is 2. In certain embodiments, o is 3. In certain embodiments, o is 6. In certain embodiments, o is 10. In certain embodiments, at least one L¹ is independently of the formula:

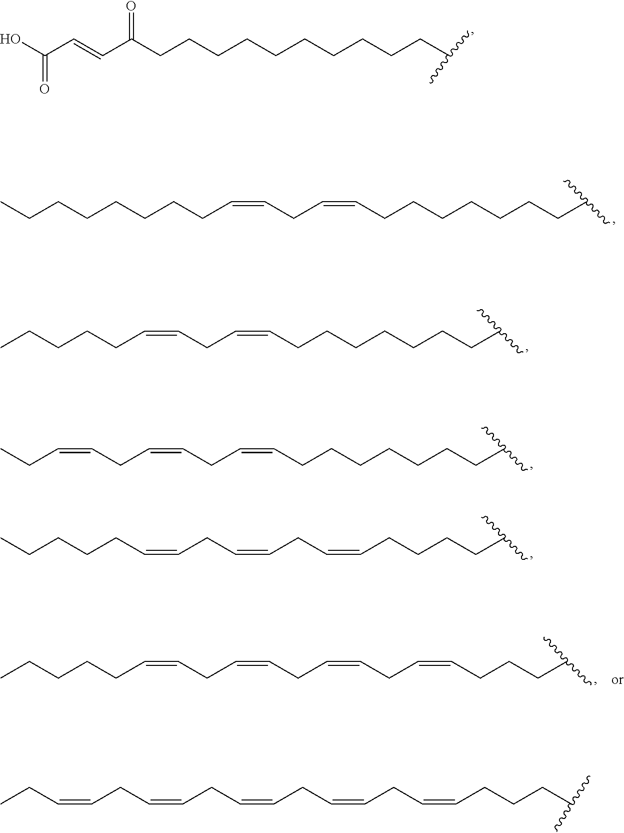

In certain embodiments, t is 1. In certain embodiments, t is 2. In certain embodiments, t is 3.

In certain embodiments,

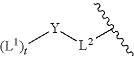

is selected from the group consisting of any of the PK/PD modulators shown in Table 2.

In certain embodiments, L⁴ is a fatty acid, fatty acid derived group, glycerolipid, glycerolipid derived group, phospholipid, phospholipid derived group, sphingolipid, sphingolipid derived group, cholesterol ester, or a cholesterol ester derived group. In certain embodiments, at least one instance of L⁴ is independently a straight chain lipid. In certain embodiments, at least one instance of L⁴ is independently a saturated lipid. In certain embodiments, at least one instance of L⁴ is independently an unsaturated lipid. In certain embodiments, at least one instance of L⁴ is independently a branched lipid. In certain embodiments, L⁴ is an NEM (i.e., N-ethylmaleimide) capping moiety.

In certain embodiments, at least one instance of L⁴ is independently of the formula:

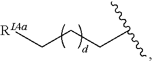

wherein R^{L4a} is H, or CO₂H; and d is an integer between 5 and 35. In certain embodiments, R^{L4a} is H. In certain embodiments, R^{L4a} IS CO₂H. In certain embodiments, d is 8, 9, 10, 11, 12, 13, 14, 15, 16, 17, 18, 19, or 20. In certain embodiments, d is 9. In certain embodiments, d is 11. In certain embodiments, d is 12. In certain embodiments, d is 13. In certain embodiments, d is 14. In certain embodiments, d is 15. In certain embodiments, d is 17.

In certain embodiments, at least one instance of L⁴ is independently of the formula

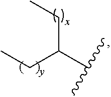

wherein x is an integer between 2 and 25; and y is an integer between 2 and 25. In certain embodiments, at least one instance of L⁴ is independently of the formula:

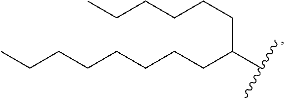 [SMILES: *C(CCCCCC)CCCCCCCC]

In certain embodiments, L⁴ is independently a straight chain lipid comprising 1-8 alkenylene moieties. In certain embodiments, at least one instance of L⁴ is independently of the formula:

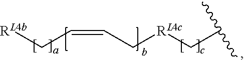

wherein R^{L4b} is —CH₃, or —CO₂H; R^{L4e} is —CH₂— or —C(O)—; a is an integer between 0 and 20; b is an integer between 1 and 8; and c is an integer between 1 and 20. In certain embodiments, R^{L4b} is —H. In certain embodiments, R^{L4b} is —CO₂H. In certain embodiments, R^{L4e} is —CH₂—. In certain embodiments, R^{L4e} is —C(O)—. In certain embodiments, a is 0. In certain embodiments, a is 1. In certain embodiments, a is 4. In certain embodiments, a is 7. In certain embodiments, b is 1. In certain embodiments, b is 2. In certain embodiments, b is 3. In certain embodiments, b is 4. In certain embodiments, b is 5. In certain embodiments, c is 2. In certain embodiments, c is 3. In certain embodiments, c is 6. In certain embodiments, c is 10. In certain embodiments, at least one L⁴ is independently of the formula:

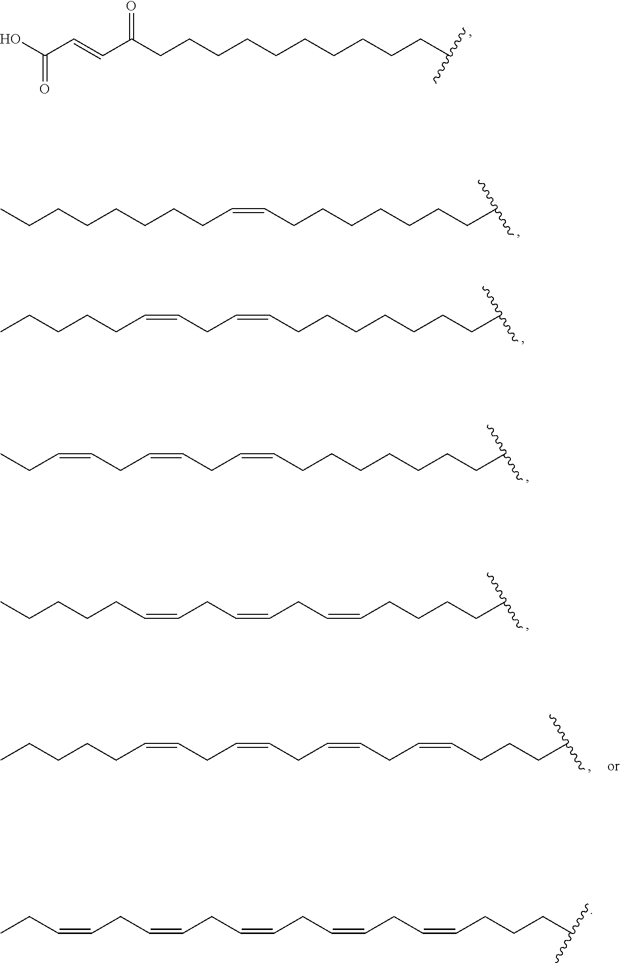

In certain embodiments, q is 1. In certain embodiments, q is 2. In certain embodiments, q is 3.

In certain embodiments,

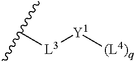

is selected from the group consisting of any of the PK/PD modulators shown in Table 2.

In some embodiments, the compounds of Formula (I) contain the substituent Y¹. In certain embodiments, Y¹ is a bond. In certain embodiments, Y¹ is a linker connecting at least one L⁴ to L³ if present, or L⁴ to R^{Z3}. In certain embodiments, Y¹ is a bivalent moiety connecting at least one L⁴ to L³ if present, or connecting L⁴ to R^{Z3}. In certain embodiments, Y¹ is of the formula:

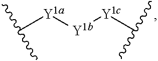

wherein Y^{1a} and Y^{1e} are each independently absent, —N(H)—, or —C(O)—; Y^{1b} is substituted or unsubstituted heteroalkylene, substituted or unsubstituted carbocyclene, substituted or unsubstituted heterocyclene; substituted or unsubstituted arylene, or substituted or unsubstituted heteroarylene. In certain embodiments, Y^{1a} is —C(O)—. In certain embodiments, Y^{1a} is —N(H)—. In certain embodiments, Y^{1c} is —C(O)—. In certain embodiments, Y^{1c} is —N(H)—. In certain embodiments, Y^{1b} is substituted or unsubstituted C₁-C₆ heteroalkylene. In certain embodiments, Y^{1b} is of the formula

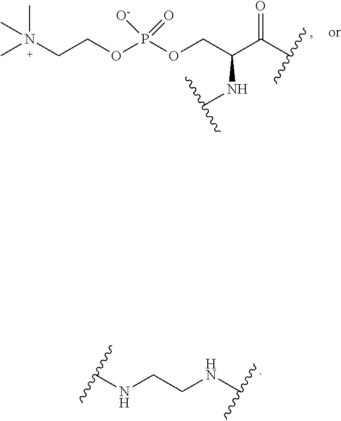

In certain embodiments, Y^{1b} substituted or unsubstituted carbocyclene. In certain embodiments, Y^{1b} is of the formula H

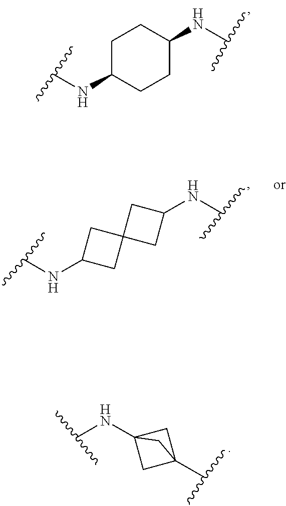 [SMILES: *N[C@H]1CC[C@@H](N*)CC1 ; *NC1CC2(C1)CC(N*)C2 ; *NC12CC(*)(C1)C2]

In certain embodiments, Y^{1b} substituted or unsubstituted heterocyclene. In certain embodiments, Y^{1b} is of the formula

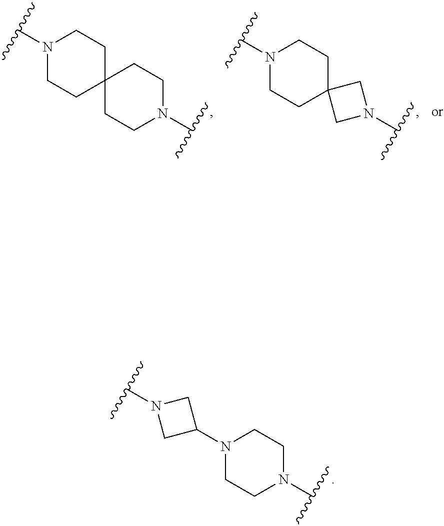 [SMILES: *N1CCC2(CC1)CCN(*)CC2 ; *N1CCC2(CC1)CN(*)C2 ; *N1CCN(C2CN(*)C2)CC1]

In certain embodiments, Y^{1b} is absent. In certain embodiments, Y^{1b} substituted or unsubstituted arylene. In certain embodiments, Y^{1b} substituted or unsubstituted phenylene. In certain embodiments, Y^{1b} is of the formula

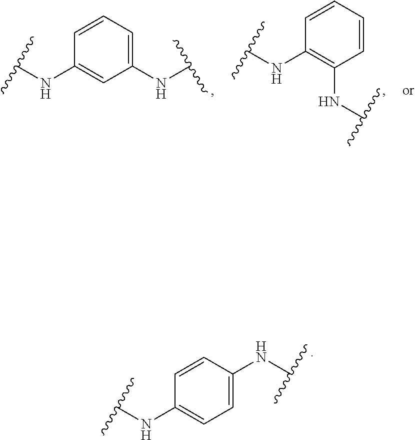 [SMILES: *Nc1cccc(N*)c1 ; *Nc1ccccc1N* ; *Nc1ccc(N*)cc1]

In certain embodiments, Y¹ is selected from the group consisting of —N(H)—C(O)—, —C(O)—N(H)—,

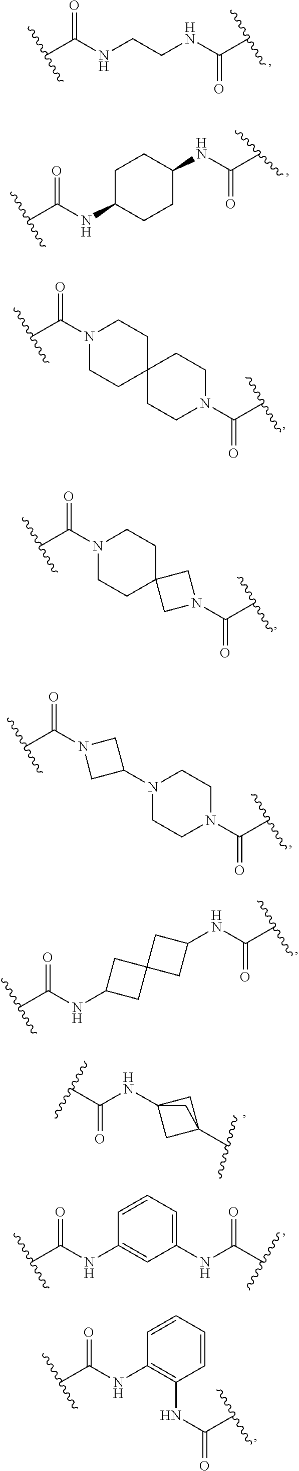

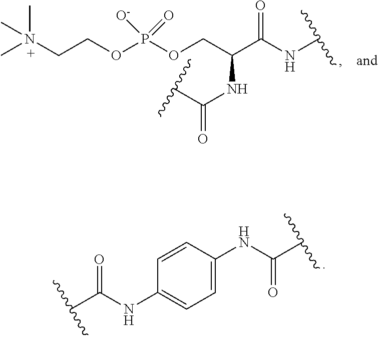

In certain embodiments, Y¹ is a trivalent moiety connecting two L⁴ groups to L³, or connecting two L⁴ groups to R^{Z3}. In certain embodiments, Y¹ is of the formula:

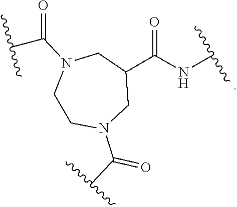 [SMILES: *NC(=O)C1CN(C(*)=O)CCN(C(*)=O)C1]

In some embodiments, Formula (I) contains the substituent L³. In certain embodiments, L³ is of the formula

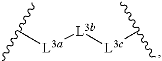

wherein:
L^{3a} is a bond or a bidentate linking group;
L^{3b} is a bond or a chemical moiety formed by reacting a first reactive moiety with a second reactive moiety; and
L^{3c} is a bond or of the formula:

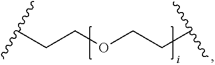

wherein i is an integer between 1 and 12.

In certain embodiments, L^{3c} is a bond. In certain embodiments, L^{3c} is of the formula:

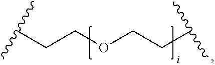

wherein h is an integer between 1 and 12. In certain embodiments, h is 2. In certain embodiments, h is 3. In certain embodiments, h is 5. In certain embodiments, h is 9. In certain embodiments, h is 10.

In certain embodiments, L^{3b} is a bond. In certain embodiments, L^{3b} is L^{3b} is a bond, —C(O)—, or of the formula:

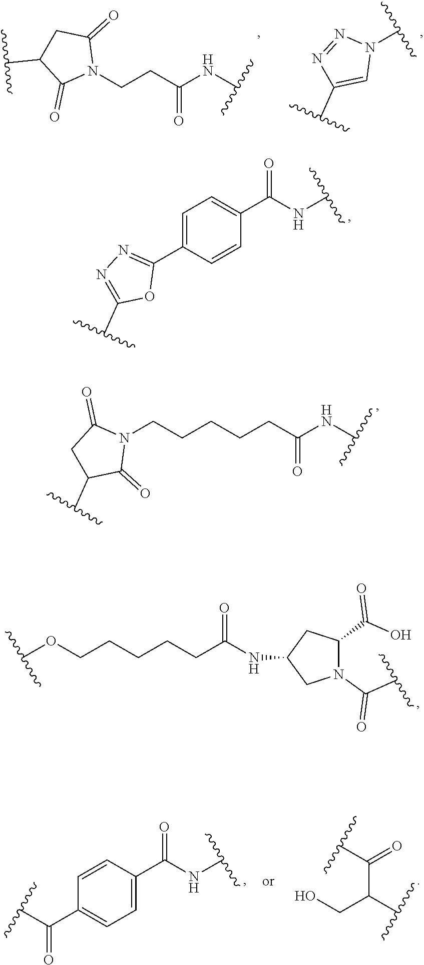

In certain embodiments, L^{3a} is of the formula:

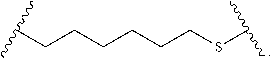 [SMILES: *CCCCCCS*]

The present disclosure further provides a lipid delivery platform for oligonucleotides, methods of using the lipid delivery platform, and methods of manufacturing the lipid delivery platform.

As used herein and as would be understood by one skilled in the art, a polyethylene glycol (PEG) unit refers to repeating units of the formula —(CH₂CH₂O)—. It will be appreciated that, in the chemical structures disclosed herein, PEG units may be depicted as —(CH₂CH₂O)—, —(OCH₂CH₂)—, or —(CH₂OCH₂)—. It will also be appreciated that a numeral indicating the number of repeating PEG units may be placed on either side of the parentheses depicting the PEG units.

Another aspect of the invention provides a process for manufacturing compounds comprising an oligonucleotide (e.g., a double-stranded or single-stranded oligonucleotide) and a lipid moiety.

In some embodiments, the method comprises conjugating an oligonucleotide-based agent comprising a first reactive moiety with a compound comprising a lipid and a second reactive moiety to form a compound comprising both an RNAi agent and a lipid moiety.

In some embodiments, the first reactive moiety is selected from the group consisting of a hydroxy and an amine reactive group. In some embodiments, the first reactive moiety is an amine. In some embodiments, the first reactive moiety is a hydroxy group. In some embodiments, the first reactive moiety is an alkyne. In some embodiments, the first reactive moiety is a disulfide.

In some embodiments, the second reactive moiety is selected from the group consisting of esters (including but not limited to activated esters such as succinimidyl esters tetrafluorophenoxy esters and para-nitrophenoxy esters), sulfones (including but not limited to methylsulfones, sulfonyl halides), maleimide, azide, and phosphoramidites. In some embodiments, the second reactive moiety is an ester. In some embodiments, the second reactive moiety is a sulfone. In some embodiments, the second reactive moiety is a phosphoramidite. In some embodiments, the second reactive moiety is a maleimide. In some embodiments, the second reactive moiety is an azide.

As shown in Table 1 below and described herein, the compounds of Formula (I), LP-4-p, LP-18-p, LP-128-p, LP-151-p, LP-183-p, LP-200-p, LP-208-p, LP-211-p, LP-232-p, LP-242-p, LP-243-p, LP-244-p, LP-245-p, LP-249-p, LP-274-p, LP-295-p, LP-310-p, LP-359-p, LP-361-p, LP-371-p, LP-374-p, LP-375-p, LP-377-p, LP-378-p, LP-379-p, LP-380-p, LP-403-p, LP-404-p, LP-412-p, LP-413-p, LP-416-p, LP-424-p, LP-425-p, LP-426-p, LP-427-p, LP-428-p, LP-432-p, LP-433-p, LP-444-p, LP-445-p, LP-446-p, LP-447-p, LP-453-p, LP-455-p, LP-457-p, LP-458-p, LP-459-p, LP-460-p, LP-461-p, LP-468-p, LP-469 phosphoramidite, LP-470 phosphoramidite, LP-473-p, LP-474-p, and CNR1 SM2 phosphoramidite, may be referred to as “pharmacokinetic and/or pharmacodynamic modulator precursors” (hereinafter, “PK/PD modulator precursors”).

It will also be appreciated that portions of said compounds may be referred to as “pharmacokinetic and/or pharmacodynamic modulators” (hereinafter, “PK/PD modulators”). When used to refer to a portion of a compound of formula LP-4-b, LP-18-b, LP-128-b, LP-151-b, LP-183-b, LP-200-b, LP-208-b, LP-211-b, LP-232-b, LP-242-b, LP-243-b, LP-244-b, LP-245-b, LP-249-b, LP-274-b, LP-295-b, LP-310-b, LP-359-b, LP-361-b, LP-371-b, LP-374-b, LP-375-b, LP-377-b, LP-378-b, LP-379-b, LP-380-b, LP-403-b, LP-404-b, LP-412-b, LP-413-b, LP-416-a, LP-416-b, LP-424-b, LP-425-b, LP-426-b, LP-427-b, LP-428-b, LP-432-b, LP-433-b, LP-444-b, LP-445-b, LP-446-b, LP-447-b, LP-453-b, LP-455-b, LP-457-b, LP-458-b, LP-459-b, LP-460-b, LP-461-b, LP-468-b, LP-469-b, LP-470-b, LP-473-b, LP-474-b, and CNR1 SM2-b, as shown in Table 3 below, the term “PK/PD modulator” refers to the portion of the compound excluding R (i.e., the oligonucleotide-based agent).

A lipid PK/PD modulator is linked to an oligonucleotide-based agent to facilitate delivery to the desired adipose cells or tissues. Lipid PK/PD modulator precursors can be synthetized having reactive moieties, including but not limited to activated ester groups and phosphoramidites, that readily facilitate linkage to one or more linking groups on an RNAi agent. Chemical reaction syntheses to link such PK/PD modulator precursors to oligonucleotides, including RNAi agents, are generally known in the art. The terms “PK/PD modulator” and “lipid PK/PD modulator” may be used interchangeably herein.

In one aspect, provided herein are compounds of Formula (II):

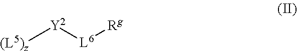

or a salt thereof, wherein:
R^{g} is a reactive moiety suitable for conjugation with an oligonucleotide-based agent;
Y² is a bond or a linker connecting at least one L⁵ to L⁶ when present, or to R^{g};
each L⁵ is independently a lipid comprising from about 10 to about 50 carbon atoms L⁶ is a linker comprising 1-20 PEG units; and
z is 1, 2, or 3, as valency permits.

The compounds of Formula (II) contain the substituent R^{g}. In certain embodiments, R^{g} is of the formula:

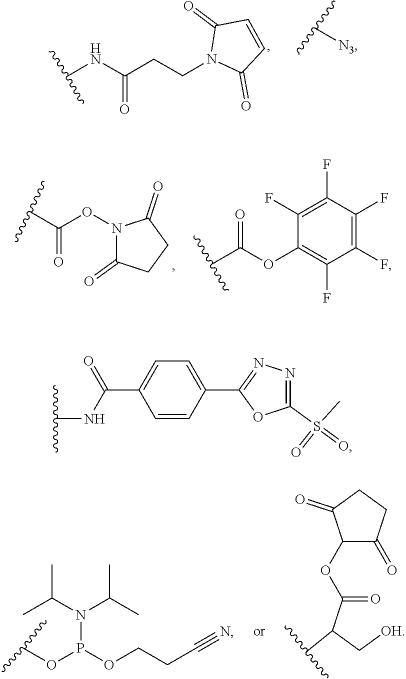

In some embodiments, the compounds of Formula (II) contain the substituent Y². In certain embodiments, Y² is a bond. In certain embodiments, Y² is a linker connecting at least one L⁵ to L⁶ if present, or L⁵ to R^{g}. In certain embodiments, Y² is a bivalent moiety connecting at least one L⁵ to L⁶ if present, or connecting L⁵ to R^{g}. In certain embodiments, Y² is selected from the group consisting of —N(H)—C(O)—, —C(O)—N(H)—,

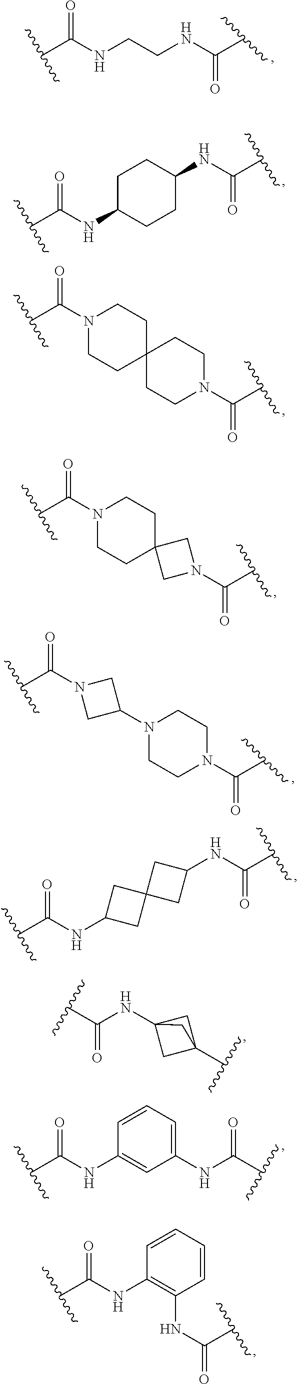

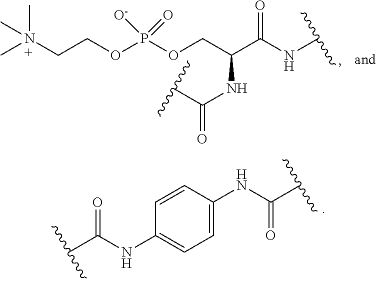

In certain embodiments, Y² is a trivalent moiety connecting two L⁵ groups to L⁶, or connecting two L⁵ groups to R^{g}. In certain embodiments, Y² is of the formula:

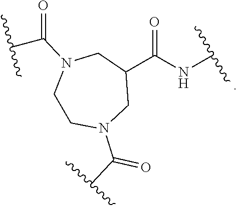 [SMILES: *NC(=O)C1CN(C(*)=O)CCN(C(*)=O)C1]

In some embodiments, the compounds of Formula (II) contain the substituent L⁶. In certain embodiments, L⁶ is a linker comprising 1-10 PEG units. In certain embodiments, L⁶ is of the formula:

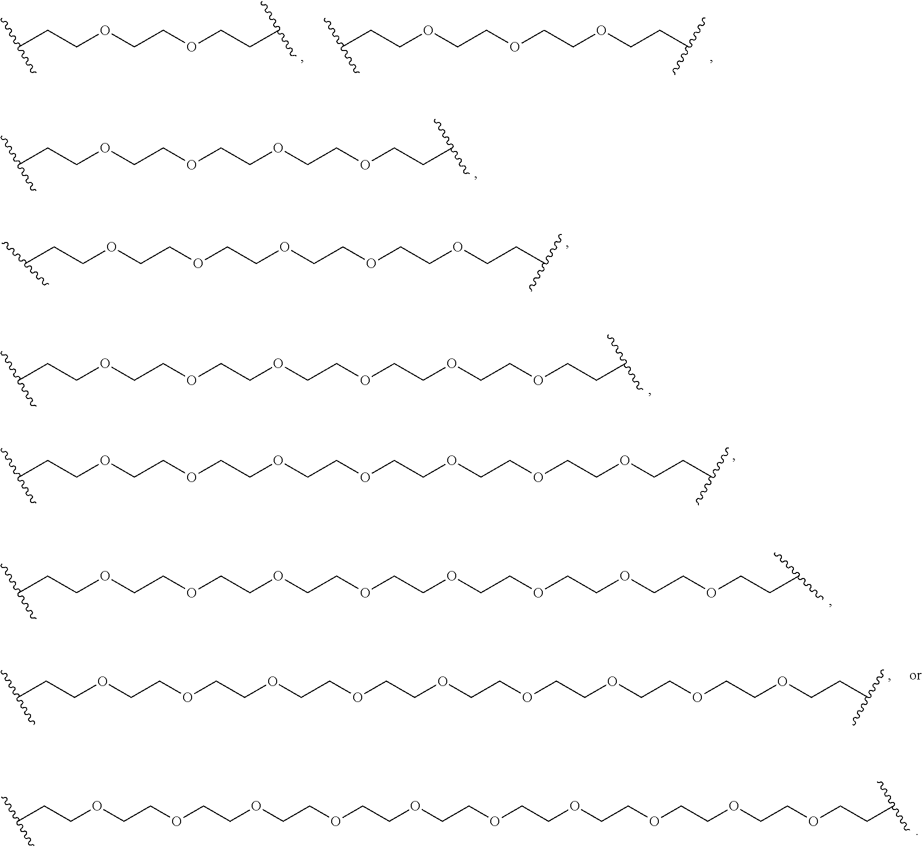

In certain embodiments, L⁶ is of the formula:

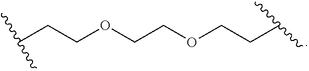 [SMILES: *CCOCCOCC*]

In certain embodiments, L⁶ is of the formula:

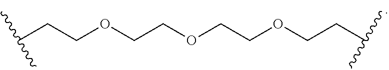 [SMILES: *CCOCCOCCOCC*]

In certain embodiments, L⁶ is of the formula:

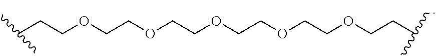 [SMILES: *CCOCCOCCOCCOCCOCC*]

In certain embodiments, L⁶ is of the formula:

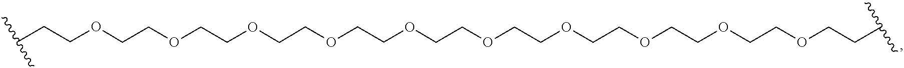 [SMILES: *CCOCCOCCOCCOCCOCCOCCOCCOCCOCCOCC*]

The compounds of Formula (II) contain the substituent L⁵. In certain embodiments, at least one instance of L⁵ is independently a saturated lipid. In certain embodiments, at least one instance of L⁵ is independently an unsaturated lipid. In certain embodiments, at least one instance of L⁵ is independently a straight chain lipid. In certain embodiments, at least one instance of L⁵ is independently a branched lipid. In certain embodiments, at least one instance of L⁵ is independently a fatty acid, fatty acid derived group, glycerolipid, glycerolipid derived group, phospholipid, phospholipid derived group, sphingolipid, sphingolipid derived group, cholesterol ester, or a cholesterol ester derived group.

In certain embodiments, at least one instance of L⁵ is independently of the formula:

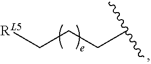

wherein R^{L5} is H, or CO₂H; and e is an integer between 5 and 35. In certain embodiments, R^{L5} is H. In certain embodiments, R^{L5} is CO₂H. In certain embodiments, e is 8, 9, 10, 11, 12, 13, 14, 15, 16, 17, 18, 19, or 20. In certain embodiments, e is 9. In certain embodiments, e is 11. In certain embodiments, e is 12. In certain embodiments, e is 13. In certain embodiments, e is 14. In certain embodiments, e is 15. In certain embodiments, e is 17.

In certain embodiments, at least one instance of L⁵ is independently of the formula:

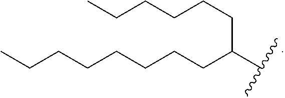 [SMILES: *C(CCCCCC)CCCCCCCC]

In certain embodiments, at least one instance of L⁵ is independently of the formula:

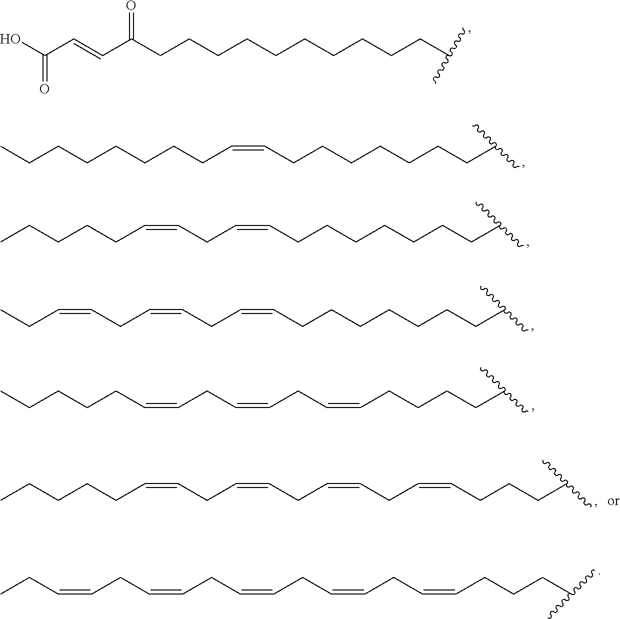

In certain embodiments, z is 1. In certain embodiments, z is 2. In certain embodiments, z is 3.

In certain embodiments, the compounds of Formula (II) may be any one of the compounds shown in table 1 below.

PK/PD modulator precursors, e.g. compounds of Formula (II), or compounds selected from the group consisting of LP-4-p, LP-18-p, LP-128-p, LP-151-p, LP-183-p, LP-200-p, LP-208-p, LP-211-p, LP-232-p, LP-242-p, LP-243-p, LP-244-p, LP-245-p, LP-249-p, LP-274-p, LP-295-p, LP-310-p, LP-359-p, LP-361-p, LP-371-p, LP-374-p, LP-375-p, LP-377-p, LP-378-p, LP-379-p, LP-380-p, LP-403-p, LP-404-p, LP-412-p, LP-413-p, LP-416-a, LP-416-b, LP-424-p, LP-425-p, LP-426-p, LP-427-p, LP-428-p, LP-432-p, LP-433-p, LP-444-p, LP-445-p, LP-446-p, LP-447-p, LP-453-p, LP-455-p, LP-457-p, LP-458-p, LP-459-p, LP-460-p, LP-461-p, LP-468-p, LP-469 phosphoramidite, LP-470 phosphoramidite, LP-473-p, LP-474-p, and CNR1 SM2 phosphoramidite, as shown in Table 1, can be used as starting materials to link to oligonucleotide-based agents such as RNAi agents and antisense oligonucleotides. The PK/PD modulator precursors may be covalently attached to an oligonucleotide-based agent using any methods known in the art. For example, in some embodiments, activated ester PK/PD modulator precursors may be reacted with an amine-containing moiety on the 5′ end of the sense strand.

TABLE 1
Lipid PK/PD modulator precursors

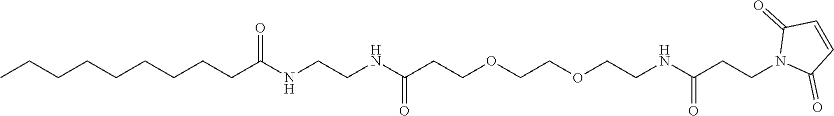 [SMILES: CCCCCCCCCC(=O)NCCNC(=O)CCOCCOCCNC(=O)CCN1C(=O)C=CC1=O]

LP-4-p

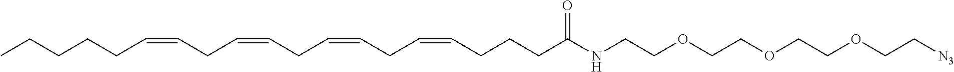 [SMILES: CCCCC/C=C\C/C=C\C/C=C\C/C=C\CCCC(=O)NCCOCCOCCOCCN=[N+]=[N-]]

LP-18-p

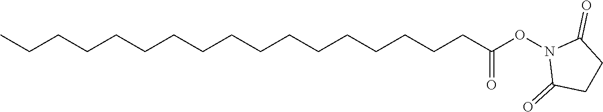 [SMILES: CCCCCCCCCCCCCCCCCC(=O)ON1C(=O)CCC1=O]

LP-128-p
(Commercially available from Santa Cruz #sc-219228)

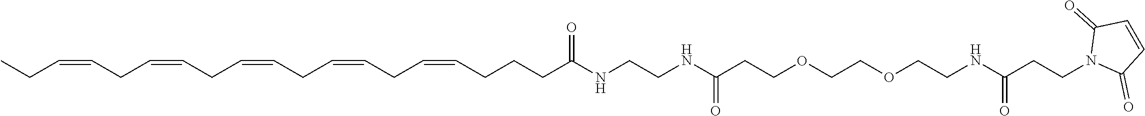 [SMILES: CC/C=C\C/C=C\C/C=C\C/C=C\C/C=C\CCCC(=O)NCCNC(=O)CCOCCOCCNC(=O)CCN1C(=O)C=CC1=O]

LP-151-p N

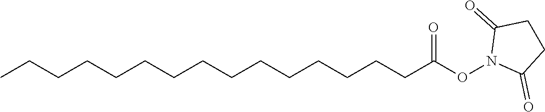 [SMILES: CCCCCCCCCCCCCCCC(=O)ON1C(=O)CCC1=O]

LP-183-p
(Commercially available from Sigma®)

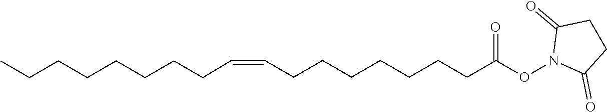 [SMILES: CCCCCCCC/C=C\CCCCCCCC(=O)ON1C(=O)CCC1=O]

LP-200-p
(Commercially available from BroadPharm®)

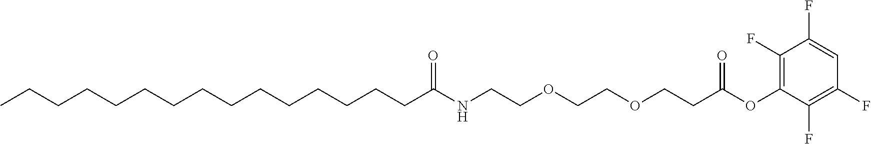 [SMILES: CCCCCCCCCCCCCCCC(=O)NCCOCCOCCC(=O)Oc1c(F)c(F)cc(F)c1F]

LP-208-p

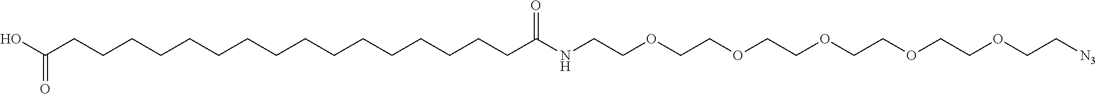 [SMILES: [N-]=[N+]=NCCOCCOCCOCCOCCOCCNC(=O)CCCCCCCCCCCCCCCCC(=O)O]

LP-211-p

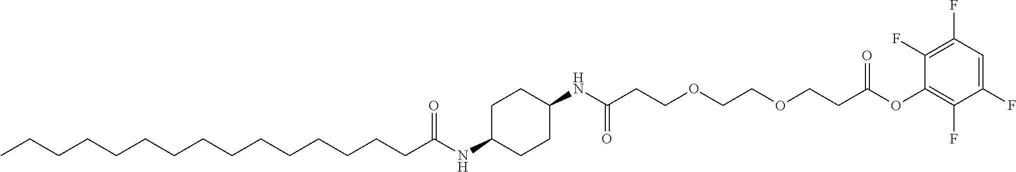 [SMILES: CCCCCCCCCCCCCCCC(=O)N[C@H]1CC[C@@H](NC(=O)CCOCCOCCC(=O)Oc2c(F)c(F)cc(F)c2F)CC1]

LP-232-p

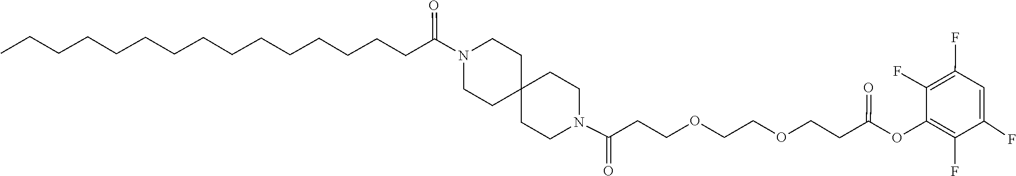 [SMILES: CCCCCCCCCCCCCCCC(=O)N1CCC2(CC1)CCN(C(=O)CCOCCOCCC(=O)Oc1c(F)c(F)cc(F)c1F)CC2]

LP-242-p

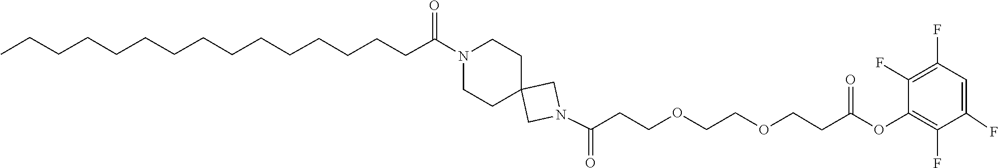 [SMILES: CCCCCCCCCCCCCCCC(=O)N1CCC2(CC1)CN(C(=O)CCOCCOCCC(=O)Oc1c(F)c(F)cc(F)c1F)C2]

LP-243-p

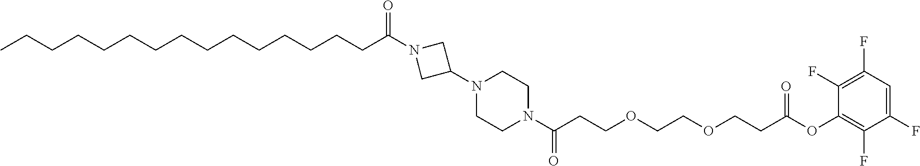 [SMILES: CCCCCCCCCCCCCCCC(=O)N1CC(N2CCN(C(=O)CCOCCOCCC(=O)Oc3c(F)c(F)cc(F)c3F)CC2)C1]

LP-244-p

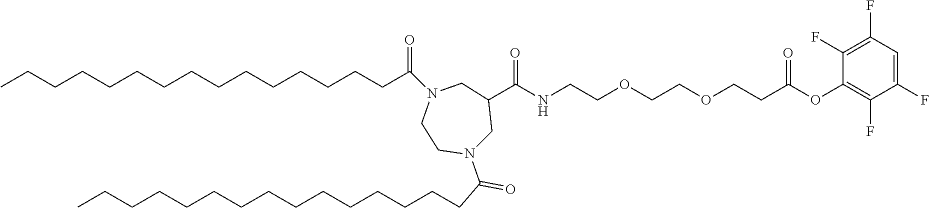 [SMILES: CCCCCCCCCCCCCCCC(=O)N1CCN(C(=O)CCCCCCCCCCCCCCC)CC(C(=O)NCCOCCOCCC(=O)Oc2c(F)c(F)cc(F)c2F)C1]

LP-245-p

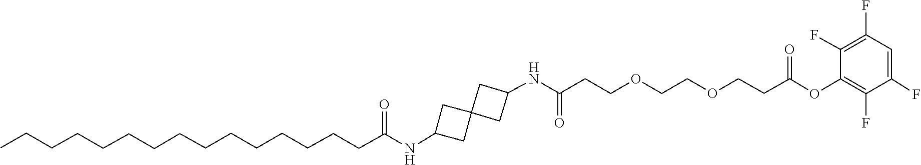 [SMILES: CCCCCCCCCCCCCCCC(=O)NC1CC2(C1)CC(NC(=O)CCOCCOCCC(=O)Oc1c(F)c(F)cc(F)c1F)C2]

LP-249-p

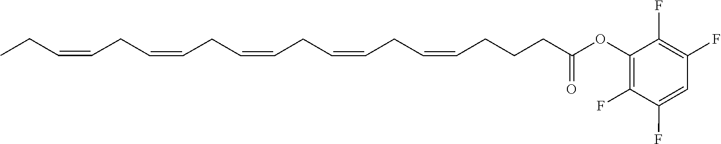 [SMILES: CC/C=C\C/C=C\C/C=C\C/C=C\C/C=C\CCCC(=O)Oc1c(F)c(F)cc(F)c1F]

LP-274-p

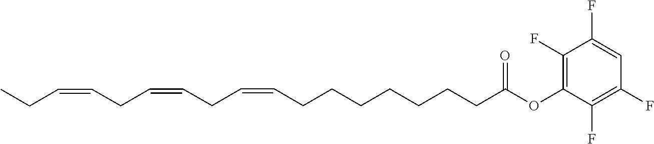 [SMILES: CC/C=C\C/C=C\C/C=C\CCCCCCCC(=O)Oc1c(F)c(F)cc(F)c1F]

LP-295-p

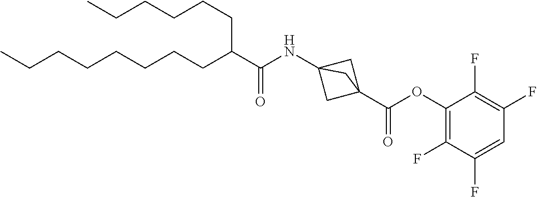 [SMILES: CCCCCCCCC(CCCCCC)C(=O)NC12CC(C(=O)Oc3c(F)c(F)cc(F)c3F)(C1)C2]

LP-310-p

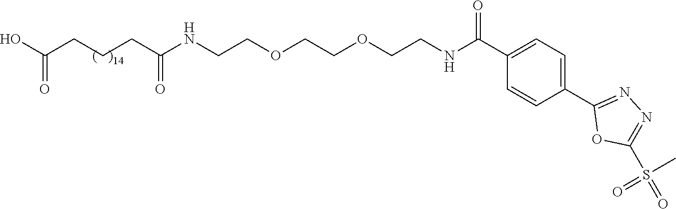

LP-359-p

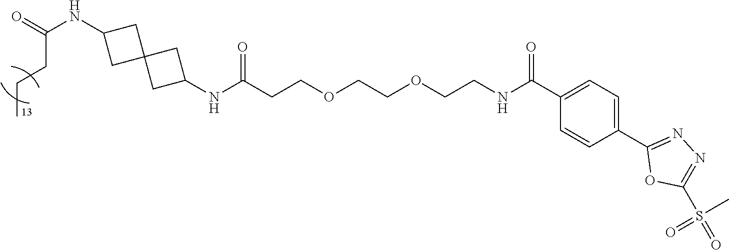

LP-361-p

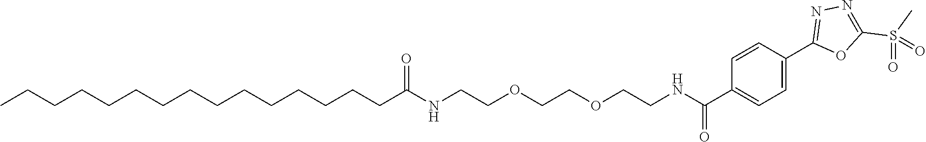 [SMILES: CCCCCCCCCCCCCCCC(=O)NCCOCCOCCNC(=O)c1ccc(-c2nnc(S(C)(=O)=O)o2)cc1]

LP-371-p

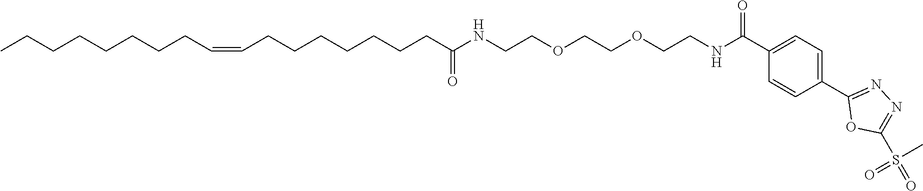 [SMILES: CCCCCCCC/C=C\CCCCCCCC(=O)NCCOCCOCCNC(=O)c1ccc(-c2nnc(S(C)(=O)=O)o2)cc1]

LP-374-p

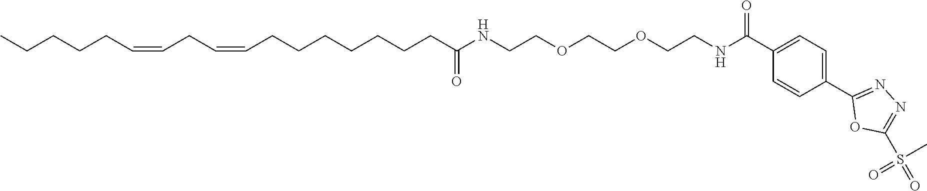 [SMILES: CCCCC/C=C\C/C=C\CCCCCCCC(=O)NCCOCCOCCNC(=O)c1ccc(-c2nnc(S(C)(=O)=O)o2)cc1]

LP-375-p

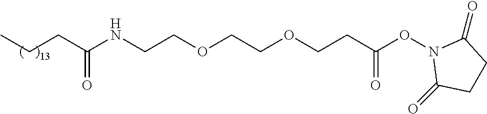

LP-377-p

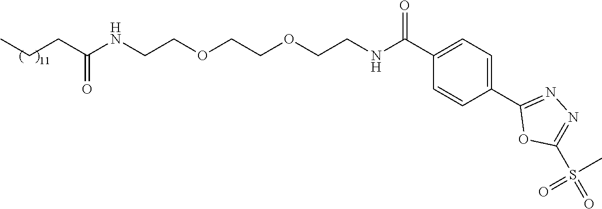

LP-378-p

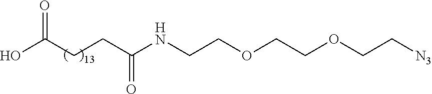

LP-379-p

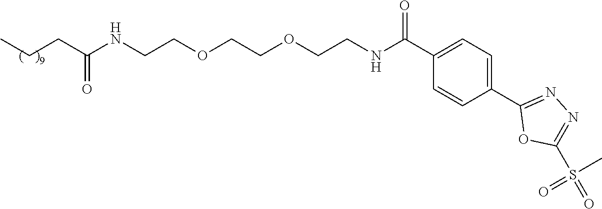

LP-380-p

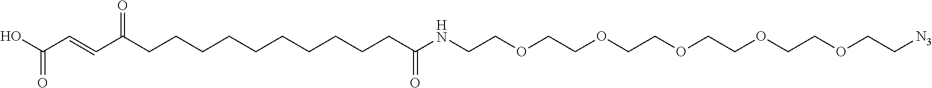 [SMILES: [N-]=[N+]=NCCOCCOCCOCCOCCOCCNC(=O)CCCCCCCCCCC(=O)/C=C/C(=O)O]

LP-403-p

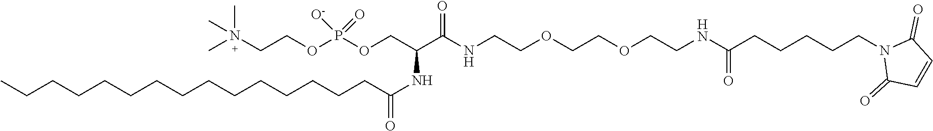

LP-404-p

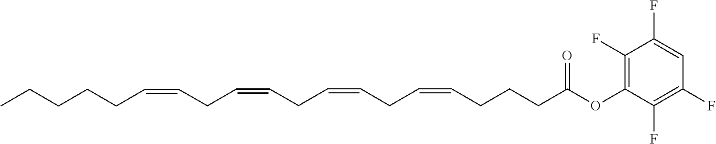 [SMILES: CCCCC/C=C\C/C=C\C/C=C\C/C=C\CCCC(=O)Oc1c(F)c(F)cc(F)c1F]

LP-412-p

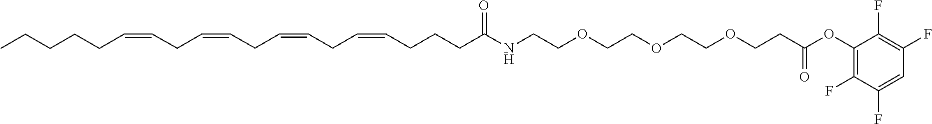 [SMILES: CCCCC/C=C\C/C=C\C/C=C\C/C=C\CCCC(=O)NCCOCCOCCOCCC(=O)Oc1c(F)c(F)cc(F)c1F]

LP-413-p

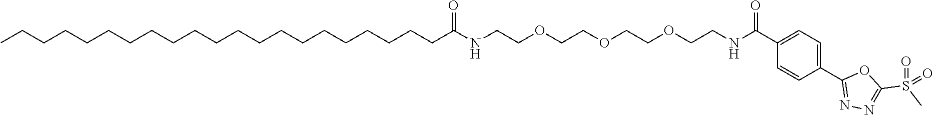 [SMILES: CCCCCCCCCCCCCCCCCCCCCC(=O)NCCOCCOCCOCCNC(=O)c1ccc(-c2nnc(S(C)(=O)=O)o2)cc1]

LP-416-p

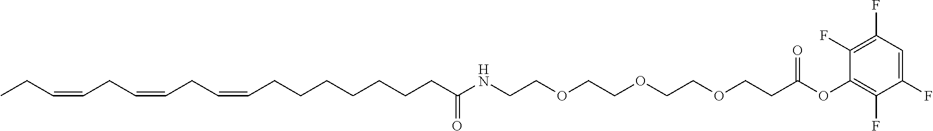 [SMILES: CC/C=C\C/C=C\C/C=C\CCCCCCCC(=O)NCCOCCOCCOCCC(=O)Oc1c(F)c(F)cc(F)c1F]

LP-424-p

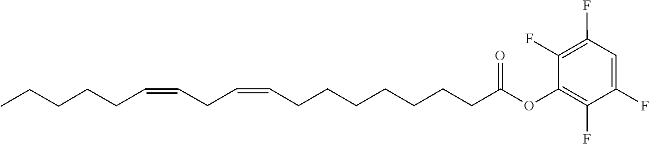 [SMILES: CCCCC/C=C\C/C=C\CCCCCCCC(=O)Oc1c(F)c(F)cc(F)c1F]

LP-425-p

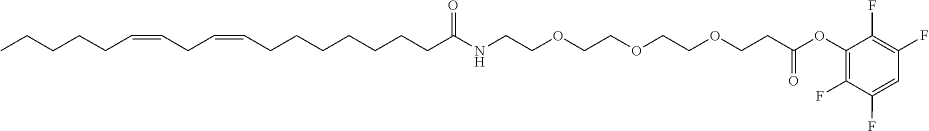 [SMILES: CCCCC/C=C\C/C=C\CCCCCCCC(=O)NCCOCCOCCOCCC(=O)Oc1c(F)c(F)cc(F)c1F]

LP-426-p

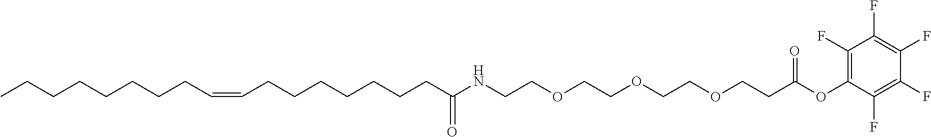 [SMILES: CCCCCCCC/C=C\CCCCCCCC(=O)NCCOCCOCCOCCC(=O)Oc1c(F)c(F)c(F)c(F)c1F]

LP-427-p

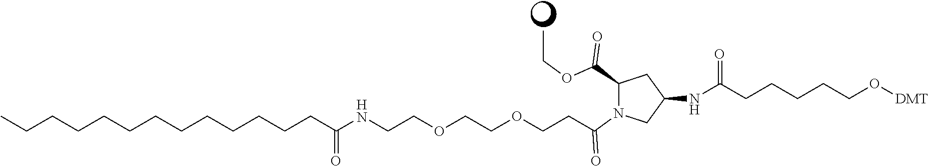

LP-428-p

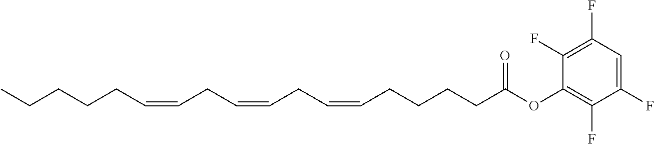 [SMILES: CCCCC/C=C\C/C=C\C/C=C\CCCCC(=O)Oc1c(F)c(F)cc(F)c1F]

LP-432-p

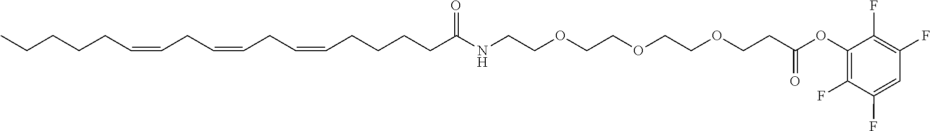 [SMILES: CCCCC/C=C\C/C=C\C/C=C\CCCCC(=O)NCCOCCOCCOCCC(=O)Oc1c(F)c(F)cc(F)c1F]

LP-433-p

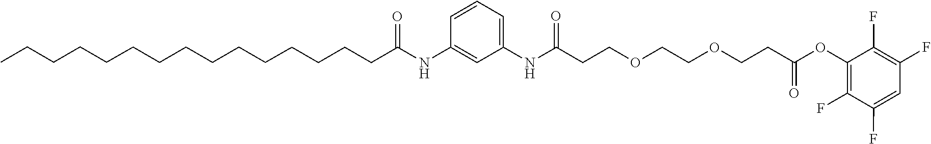 [SMILES: CCCCCCCCCCCCCCCC(=O)Nc1cccc(NC(=O)CCOCCOCCC(=O)Oc2c(F)c(F)cc(F)c2F)c1]

LP-444-p

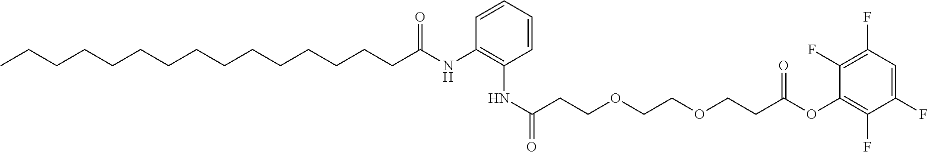 [SMILES: CCCCCCCCCCCCCCCC(=O)Nc1ccccc1NC(=O)CCOCCOCCC(=O)Oc1c(F)c(F)cc(F)c1F]

LP-445-p

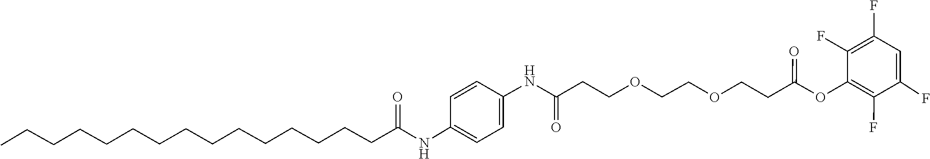 [SMILES: CCCCCCCCCCCCCCCC(=O)Nc1ccc(NC(=O)CCOCCOCCC(=O)Oc2c(F)c(F)cc(F)c2F)cc1]

LP-446-p

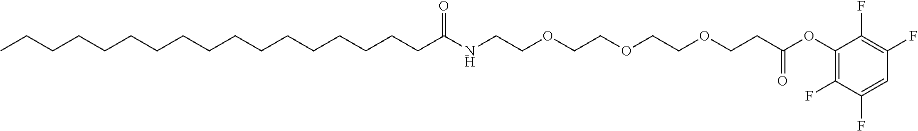 [SMILES: CCCCCCCCCCCCCCCCCC(=O)NCCOCCOCCOCCC(=O)Oc1c(F)c(F)cc(F)c1F]

LP-447-p

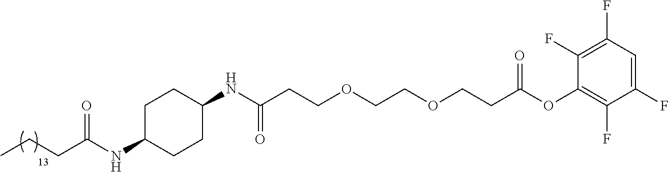

LP-453-p

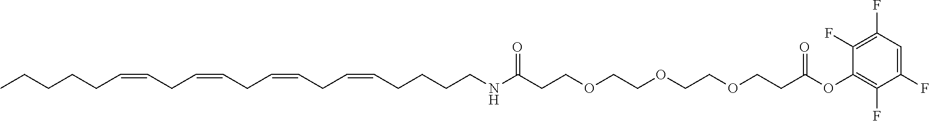 [SMILES: CCCCC/C=C\C/C=C\C/C=C\C/C=C\CCCCNC(=O)CCOCCOCCOCCC(=O)Oc1c(F)c(F)cc(F)c1F]

LP-455-p

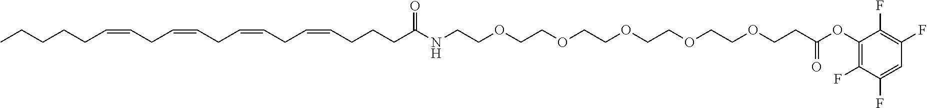 [SMILES: CCCCC/C=C\C/C=C\C/C=C\C/C=C\CCCC(=O)NCCOCCOCCOCCOCCOCCC(=O)Oc1c(F)c(F)cc(F)c1F]

LP-457-p

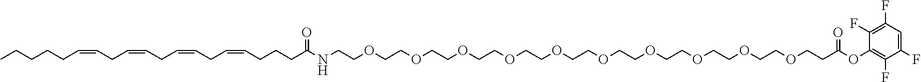 [SMILES: CCCCC/C=C\C/C=C\C/C=C\C/C=C\CCCC(=O)NCCOCCOCCOCCOCCOCCOCCOCCOCCOCCOCCC(=O)Oc1c(F)c(F)cc(F)c1F]

LP-458-p

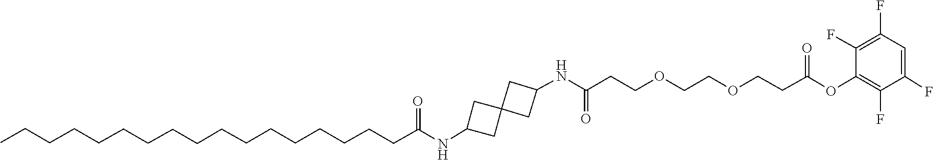 [SMILES: CCCCCCCCCCCCCCCCCC(=O)NC1CC2(C1)CC(NC(=O)CCOCCOCCC(=O)Oc1c(F)c(F)cc(F)c1F)C2]

LP-459-p

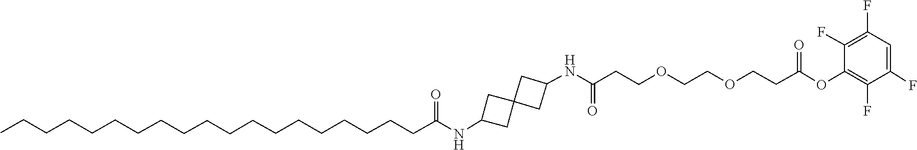 [SMILES: CCCCCCCCCCCCCCCCCCCC(=O)NC1CC2(C1)CC(NC(=O)CCOCCOCCC(=O)Oc1c(F)c(F)cc(F)c1F)C2]

LP-460-p

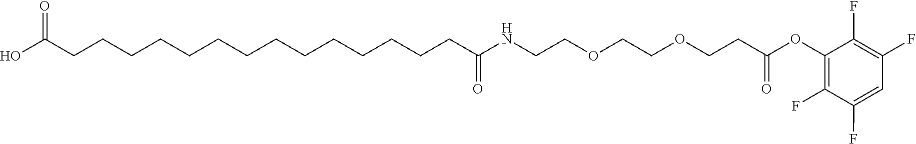 [SMILES: O=C(O)CCCCCCCCCCCCCCC(=O)NCCOCCOCCC(=O)Oc1c(F)c(F)cc(F)c1F]

LP-461-p

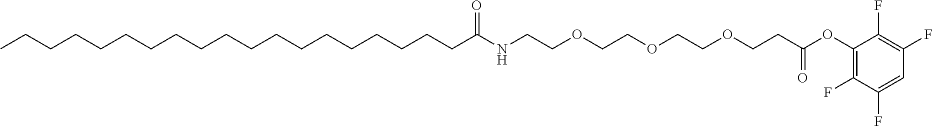 [SMILES: CCCCCCCCCCCCCCCCCCCC(=O)NCCOCCOCCOCCC(=O)Oc1c(F)c(F)cc(F)c1F]

LP-468-p

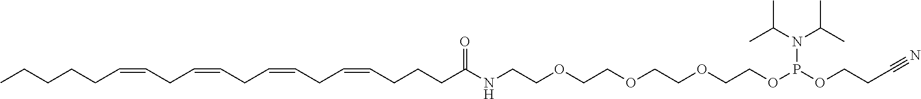 [SMILES: CCCCC/C=C\C/C=C\C/C=C\C/C=C\CCCC(=O)NCCOCCOCCOCCOP(OCCC#N)N(C(C)C)C(C)C]

LP-469 phosphoramidite

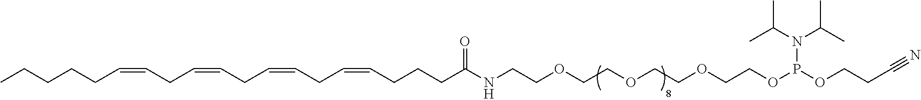

LP-470 phosphoramidite

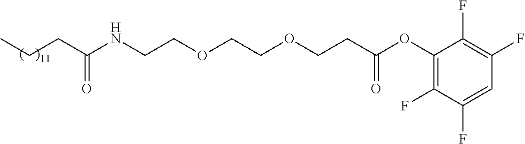

LP-473-p

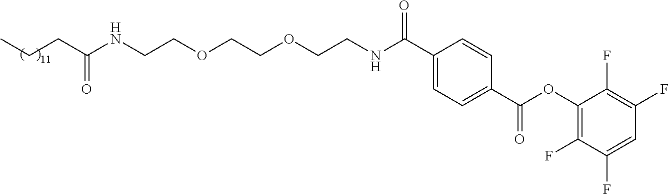

LP-474-p

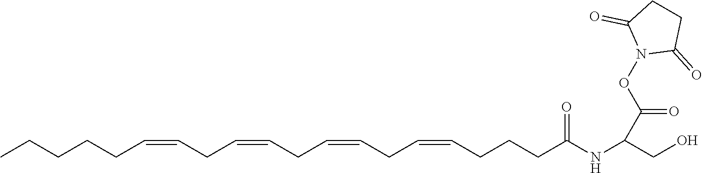 [SMILES: CCCCC/C=C\C/C=C\C/C=C\C/C=C\CCCC(=O)NC(CO)C(=O)ON1C(=O)CCC1=O]

CNR1 SM2-p

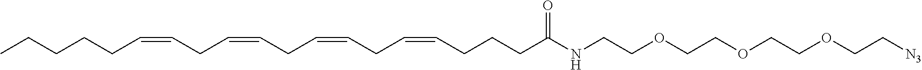 [SMILES: CCCCC/C=C\C/C=C\C/C=C\C/C=C\CCCC(=O)NCCOCCOCCOCCN=[N+]=[N-]]

CNR1 SM2-1-p

Wherein [symbol] indicates a solid support, such as a resin.

In some embodiments, one or more lipids described herein may be conjugated to an oligonucleotide-based agent, such as an RNAi agent. In some embodiments, one, two, three, four, five, six, seven or more lipids described herein may be conjugated to an oligonucleotide-based agent, such as an RNAi agent. In some embodiments, one lipid is conjugated to an oligonucleotide-based agent, such as an RNAi agent. In some embodiments, two lipids are conjugated to an oligonucleotide-based agent, such as an RNAi agent.

Lipid PK/PD modulator precursors may be conjugated to oligonucleotide-based agents using any known method in the art. In some embodiments, lipid PK/PD modulator precursors comprising an ester moiety (e.g., an activated ester) may be reacted with the oligonucleotide-based agents comprising an amine to form a compound comprising a PK/PD modulator conjugated to the oligonucleotide-based agent. An example reaction scheme is shown below

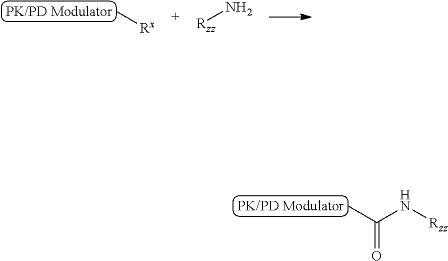

wherein R_{ZZ} comprises an RNAi agent, and R^{x} comprises an ester moiety (e.g., an activated ester such as succinimidyl esters tetrafluorophenoxy esters and para-nitrophenoxy esters).

In some embodiments, the amine may be on the 5′ or 3′ terminus of the oligonucleotide-based agent. In some embodiments, the amine may be on the 5′ terminus of the oligonucleotide-based agent. In some embodiments, the amine may be on the 3′ terminus of the oligonucleotide-based agent. In some embodiments, the oligonucleotide-based agent is an RNAi agent and the PK/PD modulator is conjugated to the sense (or passenger) strand of the RNAi agent.

In some embodiments, PK/PD modulator precursors comprising a maleimide moiety may be reacted with RNAi agents comprising a disulfide linkage to form a compound comprising a PK/PD modulator conjugated to an RNAi agent. The disulfide may be reduced, and added to a maleimide by way of a Michael-Addition reaction. An example reaction scheme is shown below:

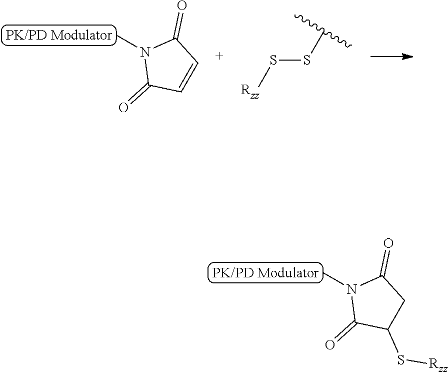

wherein R_{ZZ} comprises an RNAi agent, and [symbol] indicates a point of connection to any suitable group known in the art. In some instances of the reaction scheme above,

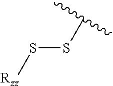

is attached to an alkyl group such as hexyl (C₆H₁₃).

In some embodiments, PK/PD modulator precursors may comprise a sulfone moiety and may react with a disulfide. An example reaction scheme is shown below:

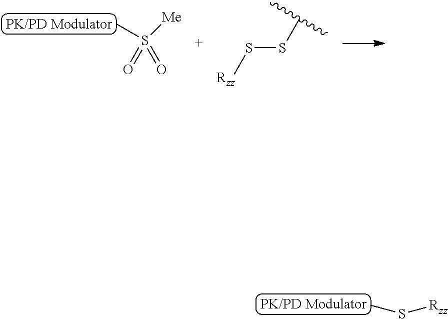

wherein R_{ZZ} comprises an RNAi agent, and

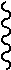

indicates a point of connection to any suitable group known in the art. In some instances of the reaction scheme above,

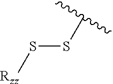

is attached to an alkyl group such as hexyl (C₆H₁₃).

In some embodiments, PK/PD modulator precursors may comprise an azide moiety and be reacted with an RNAi agent comprising an alkyne to form a compound comprising a PK/PD modulator conjugated to an RNAi agent according to the general reaction scheme below:

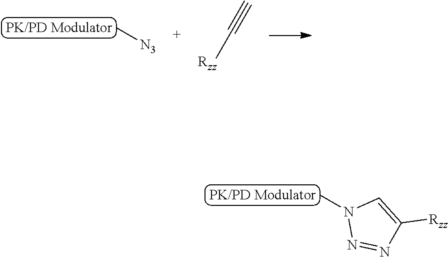

wherein R_{ZZ} comprises an RNAi agent. In some embodiments, lipid PK/PD modulator precursors comprising a sulfonyl moiety may be reacted with oligonucleotide-based agents comprising an amine to form a compound comprising a lipid PK/PD modulator conjugated to the oligonucleotide-based agent. In some embodiments, the amine may be on the 5′ or 3′ terminus of the oligonucleotide-based agent. In some embodiments, the amine may be on the 5′ terminus of the oligonucleotide-based agent. In some embodiments, the amine may be on the 3′ terminus of the oligonucleotide-based agent. In some embodiments, lipid PK/PD modulator precursors comprising a phosphoramidite moiety may be reacted with oligonucleotide-based agents comprising a hydroxyl moiety to form a compound comprising a lipid PK/PD modulator conjugated to the oligonucleotide-based agent. In some embodiments, the oligonucleotide-based agent is an RNAi agent and the hydroxyl moiety may be on the 5′ or 3′ terminus of the RNAi agent. In some embodiments, the hydroxyl moiety may be on the 5′ terminus of the RNAi agent. In some embodiments, the hydroxyl moiety may be on the 3′ terminus of the RNAi agent. In some embodiments, lipid PK/PD modulator precursors comprising an azide may be reacted with oligonucleotide-based agents comprising an alkyne to form a compound comprising a lipid PK/PD modulator conjugated to the oligonucleotide-based agent. In some embodiments, the alkyne may be on the 5′ or 3′ terminus of the oligonucleotide-based agent. In some embodiments, the alkyne may be on the 5′ terminus of the oligonucleotide-based agent. In some embodiments, the alkyne may be on the 3′ terminus of the oligonucleotide-based agent.

In some embodiments, lipid PK/PD modulators may be conjugated to the 5′ end of the sense or antisense strand, the 3′ end of the sense or antisense strand, or to an internal nucleotide of RNAi agents. In some embodiments, lipid PK/PD modulators may be conjugated to the 2′ position of a nucleotide of the sense or antisense strand. For example, lipid PK/KD modulators can be conjugated to an oligonucleotide-based agent that comprises a modified nucleotide containing an alkyne moiety (e.g., aAlk, aAlks, cAlk, cAlks, gAlk, gAlks, uAlk, uAlks; see table 4) by contacting said alkyne with PK/PD modulator precursor comprising an azide moiety.

In some embodiments, an RNAi agent is synthesized with a disulfide-containing moiety at the 3′ end of the sense strand, and a lipid PK/PD modulator precursor may be conjugated to the 3′ end of the sense strand using any of the appropriate general synthetic schemes shown above.

In some embodiments, Lipid PK/PD modulators include compounds shown in Table 2.

TABLE 2
Lipid PK/PD modulators

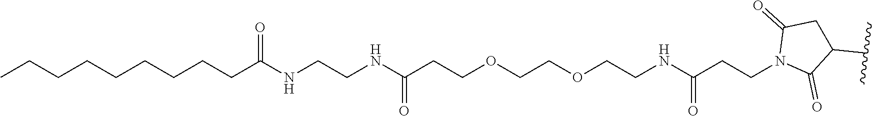 [SMILES: *C1CC(=O)N(CCC(=O)NCCOCCOCCC(=O)NCCNC(=O)CCCCCCCCC)C1=O]

LP-4-a

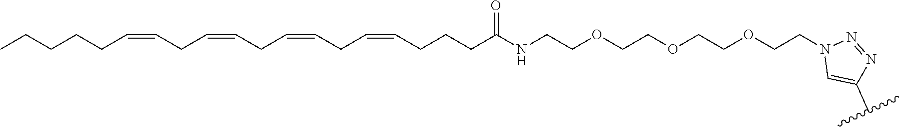 [SMILES: *c1cn(CCOCCOCCOCCNC(=O)CCC/C=C\C/C=C\C/C=C\C/C=C\CCCCC)nn1]

LP-18-a

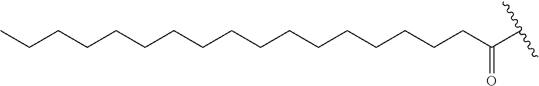 [SMILES: *C(=O)CCCCCCCCCCCCCCCCC]

LP-128-a

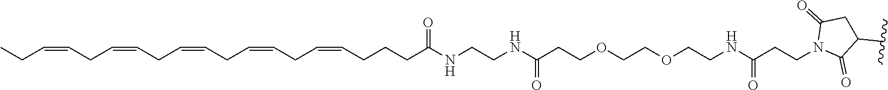 [SMILES: *C1CC(=O)N(CCC(=O)NCCOCCOCCC(=O)NCCNC(=O)CCC/C=C\C/C=C\C/C=C\C/C=C\C/C=C\CC)C1=O]

LP-151-a

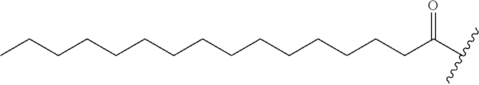 [SMILES: *C(=O)CCCCCCCCCCCCCCC]

LP-183-a

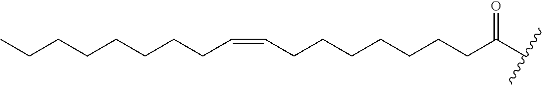 [SMILES: *C(=O)CCCCCCC/C=C\CCCCCCCC]

LP-200-a

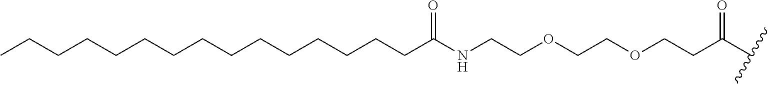 [SMILES: *C(=O)CCOCCOCCNC(=O)CCCCCCCCCCCCCCC]

LP-208-a

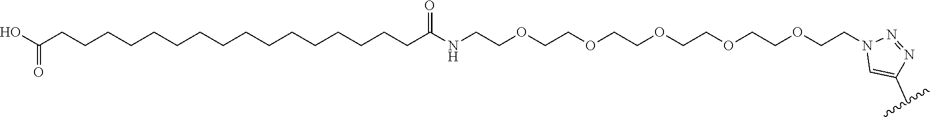 [SMILES: *c1cn(CCOCCOCCOCCOCCOCCNC(=O)CCCCCCCCCCCCCCCCC(=O)O)nn1]

LP-211-a

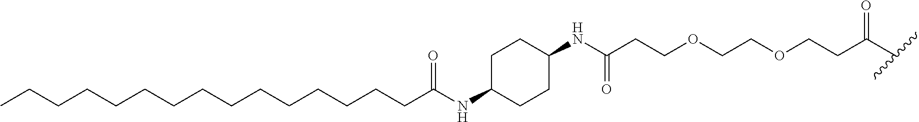 [SMILES: *C(=O)CCOCCOCCC(=O)N[C@H]1CC[C@@H](NC(=O)CCCCCCCCCCCCCCC)CC1]

LP-232-a

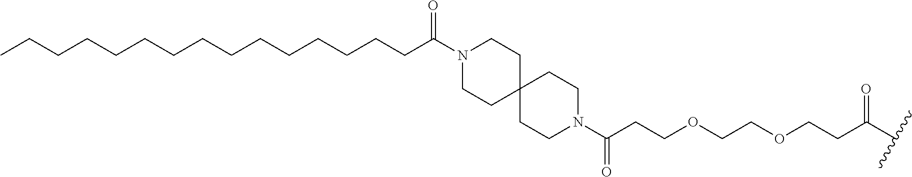 [SMILES: *C(=O)CCOCCOCCC(=O)N1CCC2(CCN(C(=O)CCCCCCCCCCCCCCC)CC2)CC1]

LP-242-a

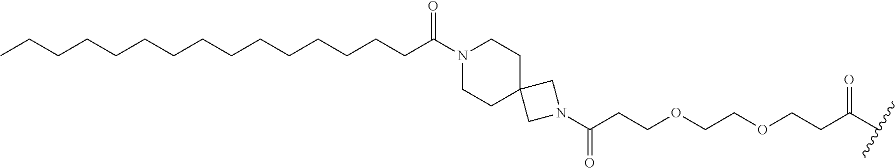 [SMILES: *C(=O)CCOCCOCCC(=O)N1CC2(CCN(C(=O)CCCCCCCCCCCCCCC)CC2)C1]

LP-243-a

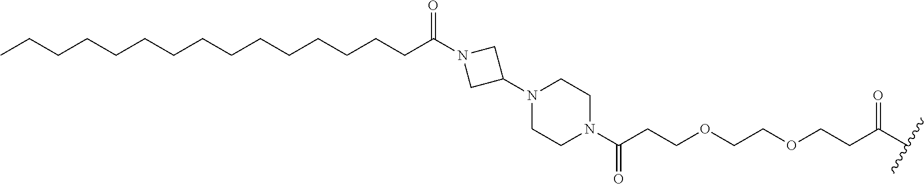 [SMILES: *C(=O)CCOCCOCCC(=O)N1CCN(C2CN(C(=O)CCCCCCCCCCCCCCC)C2)CC1]

LP-244-a

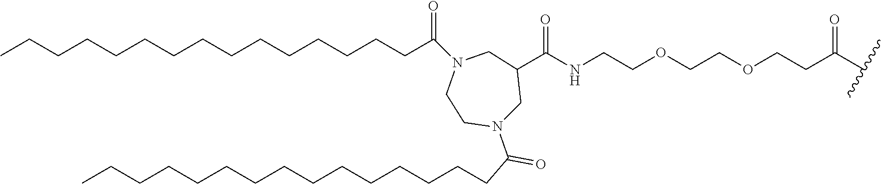 [SMILES: *C(=O)CCOCCOCCNC(=O)C1CN(C(=O)CCCCCCCCCCCCCCC)CCN(C(=O)CCCCCCCCCCCCCCC)C1]

LP-245-a

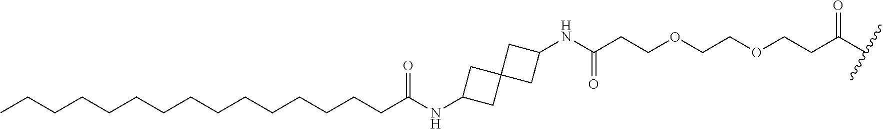 [SMILES: *C(=O)CCOCCOCCC(=O)NC1CC2(CC(NC(=O)CCCCCCCCCCCCCCC)C2)C1]

LP-249-a

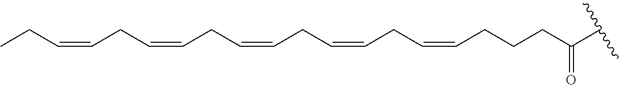 [SMILES: *C(=O)CCC/C=C\C/C=C\C/C=C\C/C=C\C/C=C\CC]

LP-274-a

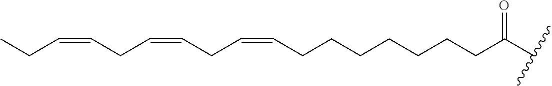 [SMILES: *C(=O)CCCCCCC/C=C\C/C=C\C/C=C\CC]

LP-295-a

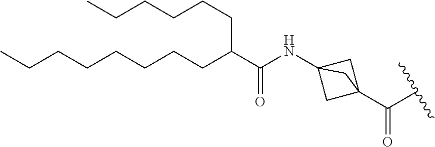 [SMILES: *C(=O)C12CC(NC(=O)C(CCCCCC)CCCCCCCC)(C1)C2]

LP-310-a

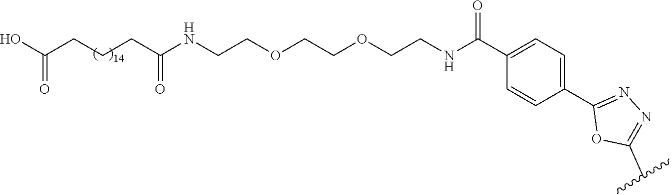

LP-359-a

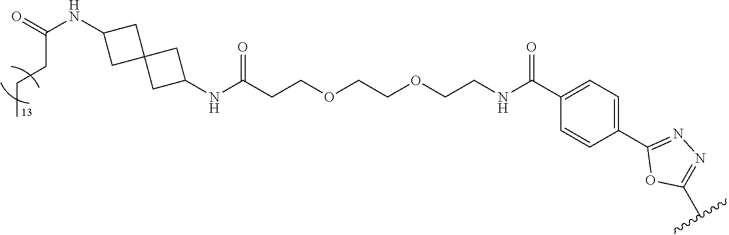

LP-361-a

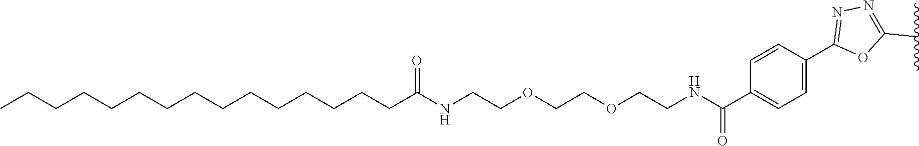 [SMILES: *c1nnc(-c2ccc(C(=O)NCCOCCOCCNC(=O)CCCCCCCCCCCCCCC)cc2)o1]

LP-371-a

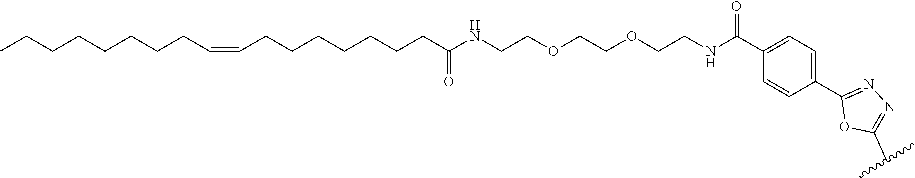 [SMILES: *c1nnc(-c2ccc(C(=O)NCCOCCOCCNC(=O)CCCCCCC/C=C\CCCCCCCC)cc2)o1]

LP-374-a

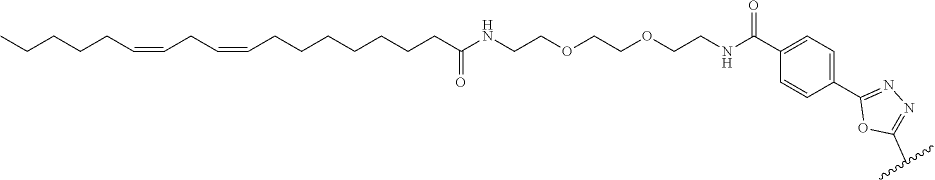 [SMILES: *c1nnc(-c2ccc(C(=O)NCCOCCOCCNC(=O)CCCCCCC/C=C\C/C=C\CCCCC)cc2)o1]

LP-375-a

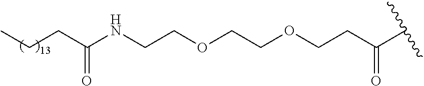

LP-377-a

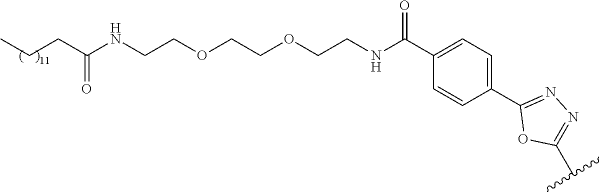

LP-378-a

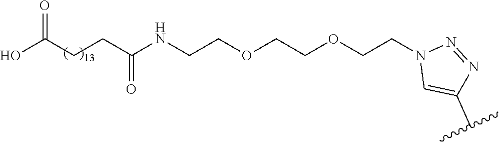

LP-379-a

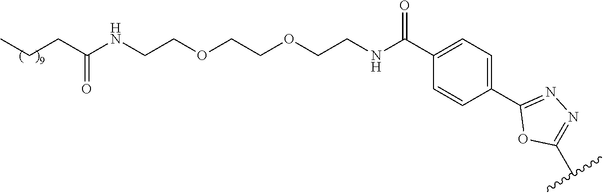

LP-380-a

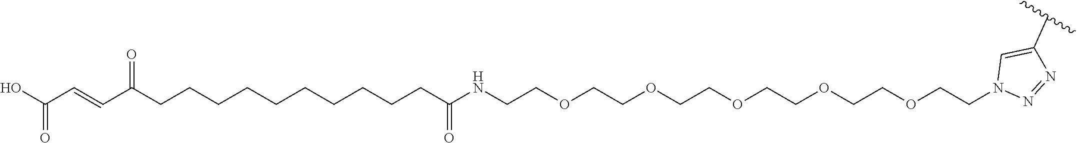 [SMILES: *c1cn(CCOCCOCCOCCOCCOCCNC(=O)CCCCCCCCCCC(=O)/C=C/C(=O)O)nn1]

LP-403-a

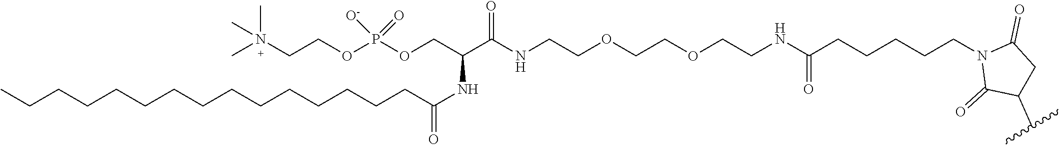

LP-404-a

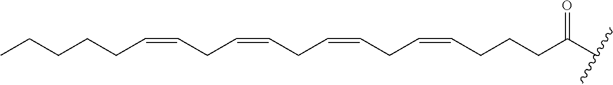 [SMILES: *C(=O)CCC/C=C\C/C=C\C/C=C\C/C=C\CCCCC]

LP-412-a

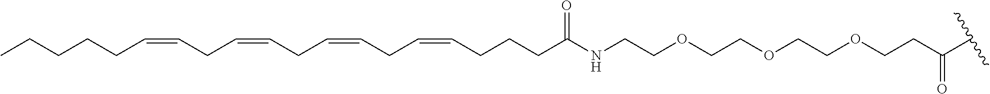 [SMILES: *C(=O)CCOCCOCCOCCNC(=O)CCC/C=C\C/C=C\C/C=C\C/C=C\CCCCC]

LP-413-a

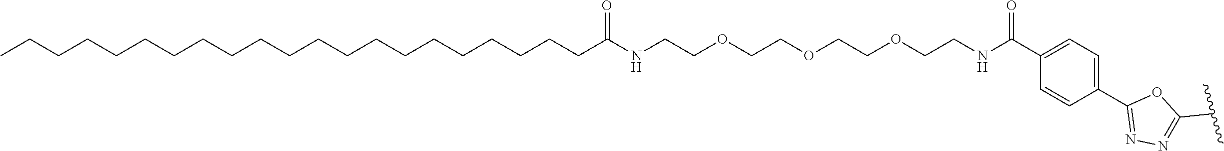 [SMILES: *c1nnc(-c2ccc(C(=O)NCCOCCOCCOCCNC(=O)CCCCCCCCCCCCCCCCCCCCC)cc2)o1]

LP-416-a

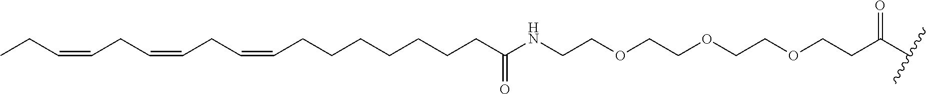 [SMILES: *C(=O)CCOCCOCCOCCNC(=O)CCCCCCC/C=C\C/C=C\C/C=C\CC]

LP-424-a

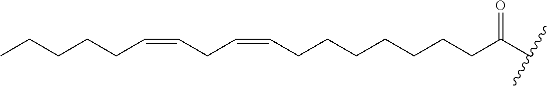 [SMILES: *C(=O)CCCCCCC/C=C\C/C=C\CCCCC]

LP-425-a

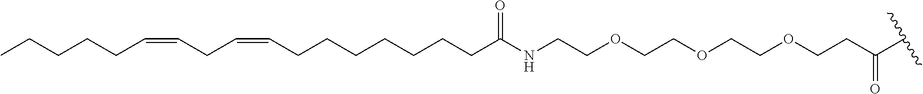 [SMILES: *C(=O)CCOCCOCCOCCNC(=O)CCCCCCC/C=C\C/C=C\CCCCC]

LP-426-a

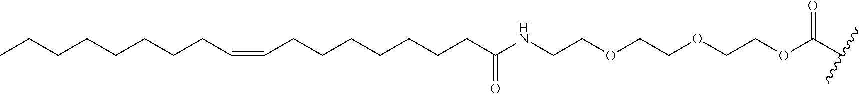 [SMILES: *C(=O)OCCOCCOCCNC(=O)CCCCCCC/C=C\CCCCCCCC]

LP-427-a

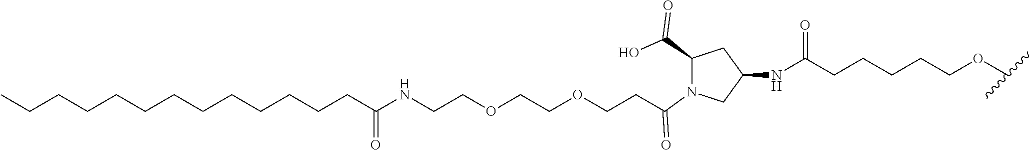 [SMILES: *OCCCCCC(=O)N[C@@H]1C[C@H](C(=O)O)N(C(=O)CCOCCOCCNC(=O)CCCCCCCCCCCCC)C1]

LP-428-a

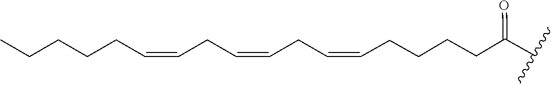 [SMILES: *C(=O)CCCC/C=C\C/C=C\C/C=C\CCCCC]

LP-432-a

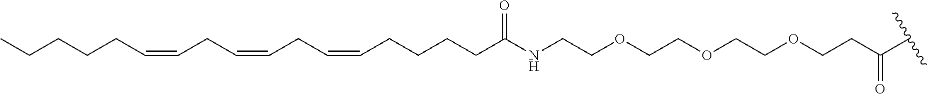 [SMILES: *C(=O)CCOCCOCCOCCNC(=O)CCCC/C=C\C/C=C\C/C=C\CCCCC]

LP-433-a

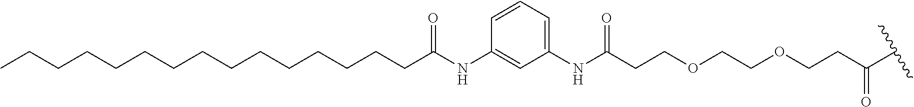 [SMILES: *C(=O)CCOCCOCCC(=O)Nc1cccc(NC(=O)CCCCCCCCCCCCCCC)c1]

LP-444-a

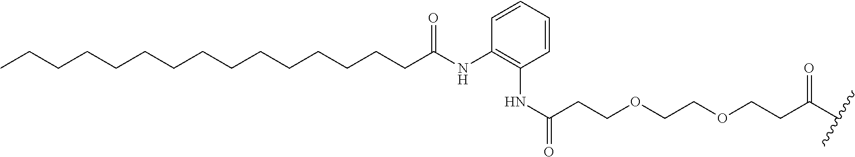 [SMILES: *C(=O)CCOCCOCCC(=O)Nc1ccccc1NC(=O)CCCCCCCCCCCCCCC]

LP-445-a

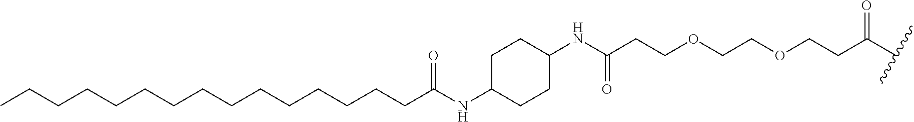 [SMILES: *C(=O)CCOCCOCCC(=O)NC1CCC(NC(=O)CCCCCCCCCCCCCCC)CC1]

LP-446-a

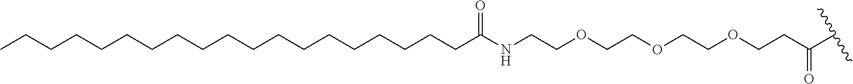 [SMILES: *C(=O)CCOCCOCCOCCNC(=O)CCCCCCCCCCCCCCCCCCC]

LP-447-a

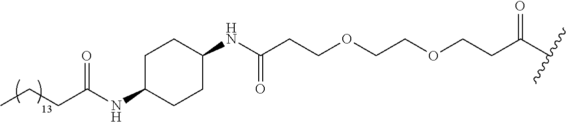

LP-453-a

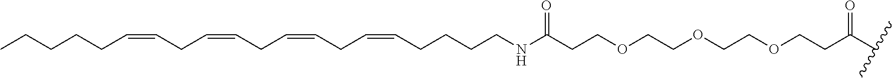 [SMILES: *C(=O)CCOCCOCCOCCC(=O)NCCCC/C=C\C/C=C\C/C=C\C/C=C\CCCCC]

LP-455-a

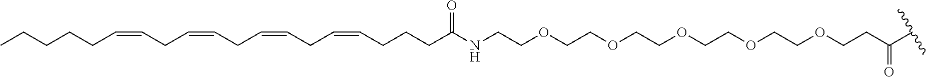 [SMILES: *C(=O)CCOCCOCCOCCOCCOCCNC(=O)CCC/C=C\C/C=C\C/C=C\C/C=C\CCCCC]

LP-457-a

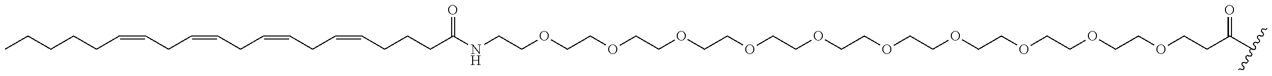 [SMILES: *C(=O)CCOCCOCCOCCOCCOCCOCCOCCOCCOCCOCCNC(=O)CCC/C=C\C/C=C\C/C=C\C/C=C\CCCCC]

LP-458-a

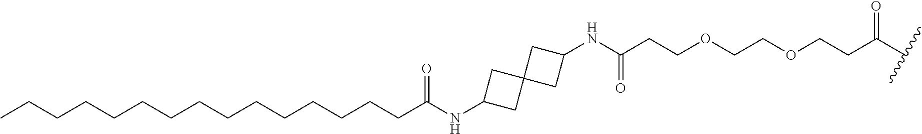 [SMILES: *C(=O)CCOCCOCCC(=O)NC1CC2(CC(NC(=O)CCCCCCCCCCCCCCC)C2)C1]

LP-459-a

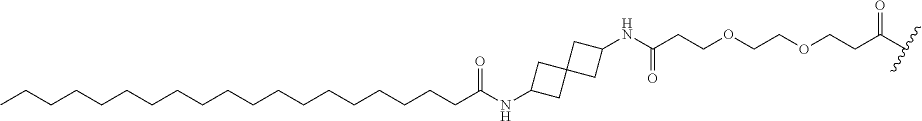 [SMILES: *C(=O)CCOCCOCCC(=O)NC1CC2(CC(NC(=O)CCCCCCCCCCCCCCCCCCC)C2)C1]

LP-460-a

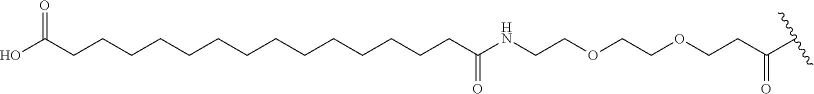 [SMILES: *C(=O)CCOCCOCCNC(=O)CCCCCCCCCCCCCCC(=O)O]

LP-461-a

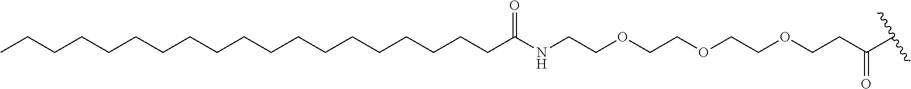 [SMILES: *C(=O)CCOCCOCCOCCNC(=O)CCCCCCCCCCCCCCCCCCC]

LP-468-a

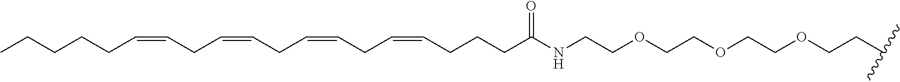 [SMILES: *CCOCCOCCOCCNC(=O)CCC/C=C\C/C=C\C/C=C\C/C=C\CCCCC]

LP-469-a

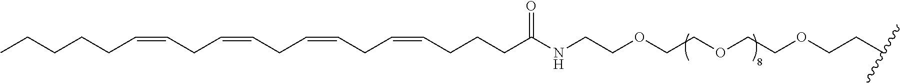

LP-470-a

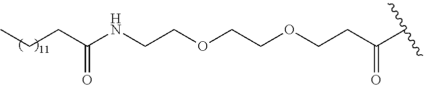

LP-473-a

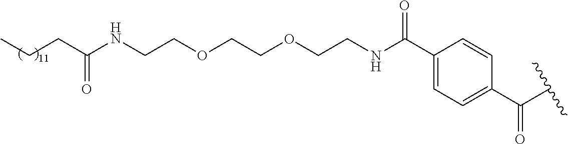

LP-474-a

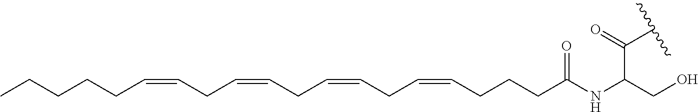 [SMILES: *C(=O)C(CO)NC(=O)CCC/C=C\C/C=C\C/C=C\C/C=C\CCCCC]

CNR1 SM2-a

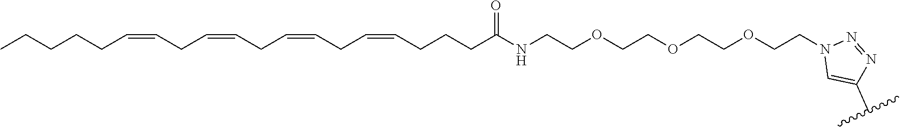 [SMILES: *c1cn(CCOCCOCCOCCNC(=O)CCC/C=C\C/C=C\C/C=C\C/C=C\CCCCC)nn1]

CNR1 SM2-1-a

wherein [symbol] indicates the point of connection to an oligonucleotide.

Each of the lipid PK/PD modulators described herein may be conjugated to an oligonucleotide on the 5′ terminus, the 3′ terminus, or both. Each of the lipid PK/PD modulators described herein may be combined with any one of the other lipid PK/PD modulators described herein. In some embodiments the lipid PK/PD modulators may be directly conjugated to the 5′ or 3′ terminal nucleotide of an oligonucleotide, and in other embodiments a linker may be used to conjugate a lipid PK/PD modulator to a terminal nucleotide (e.g., the linker L6—see table 4 for structural information.) As shown in Table 2B, any combination of lipid PK/PD modulators may be used to synthesize an oligonucleotide-based agent.

TABLE 2B
Combinations of Lipid PK/PD modulators (3′: the lipid
PK/PD modulator is conjugated to the 3′ end of the
oligonucleotide-based agent; 5′: the lipid PK/PD modulator
is conjugated to the 5′ end of the oligonucleotide-based agent.)

| 5′ | 3′ |
|---|---|
| LP-4-a | LP-4-a |
| LP-4-a | LP-18-a |
| LP-4-a | LP-128-a |
| LP-4-a | LP-151-a |
| LP-4-a | LP-183-a |
| LP-4-a | LP-200-a |
| LP-4-a | LP-208-a |
| LP-4-a | LP-211-a |
| LP-4-a | LP-232-a |
| LP-4-a | LP-242-a |
| LP-4-a | LP-243-a |
| LP-4-a | LP-244-a |
| LP-4-a | LP-245-a |
| LP-4-a | LP-249-a |
| LP-4-a | LP-274-a |
| LP-4-a | LP-295-a |
| LP-4-a | LP-310-a |
| LP-4-a | LP-359-a |
| LP-4-a | LP-361-a |
| LP-4-a | LP-371-a |
| LP-4-a | LP-374-a |
| LP-4-a | LP-375-a |
| LP-4-a | LP-377-a |
| LP-4-a | LP-378-a |
| LP-4-a | LP-379-a |
| LP-4-a | LP-380-a |
| LP-4-a | LP-403-a |
| LP-4-a | LP-404-a |
| LP-4-a | LP-412-a |
| LP-4-a | LP-413-a |
| LP-4-a | LP-424-a |
| LP-4-a | LP-425-a |
| LP-4-a | LP-426-a |
| LP-4-a | LP-427-a |
| LP-4-a | LP-428-a |
| LP-4-a | LP-432-a |
| LP-4-a | LP-433-a |
| LP-4-a | LP-444-a |
| LP-4-a | LP-445-a |
| LP-4-a | LP-446-a |
| LP-4-a | LP-447-a |
| LP-4-a | LP-453-a |
| LP-4-a | LP-455-a |
| LP-4-a | LP-457-a |
| LP-4-a | LP-458-a |
| LP-4-a | LP-459-a |
| LP-4-a | LP-460-a |
| LP-4-a | LP-461-a |
| LP-4-a | LP-468-a |
| LP-4-a | LP-469-a |
| LP-4-a | LP-470-a |
| LP-4-a | LP-473-a |
| LP-4-a | LP-474-a |
| LP-4-a | CNR1 SM2-a |
| LP-4-a | (None) |
| LP-18-a | LP-4-a |
| LP-18-a | LP-18-a |
| LP-18-a | LP-128-a |
| LP-18-a | LP-151-a |
| LP-18-a | LP-183-a |
| LP-18-a | LP-200-a |
| LP-18-a | LP-208-a |
| LP-18-a | LP-211-a |
| LP-18-a | LP-232-a |
| LP-18-a | LP-242-a |
| LP-18-a | LP-243-a |
| LP-18-a | LP-244-a |
| LP-18-a | LP-245-a |
| LP-18-a | LP-249-a |
| LP-18-a | LP-274-a |
| LP-18-a | LP-295-a |
| LP-18-a | LP-310-a |
| LP-18-a | LP-359-a |
| LP-18-a | LP-361-a |
| LP-18-a | LP-371-a |
| LP-18-a | LP-374-a |
| LP-18-a | LP-375-a |
| LP-18-a | LP-377-a |
| LP-18-a | LP-378-a |
| LP-18-a | LP-379-a |
| LP-18-a | LP-380-a |
| LP-18-a | LP-403-a |
| LP-18-a | LP-404-a |
| LP-18-a | LP-412-a |
| LP-18-a | LP-413-a |
| LP-18-a | LP-424-a |
| LP-18-a | LP-425-a |
| LP-18-a | LP-426-a |
| LP-18-a | LP-427-a |
| LP-18-a | LP-428-a |
| LP-18-a | LP-432-a |
| LP-18-a | LP-433-a |
| LP-18-a | LP-444-a |
| LP-18-a | LP-445-a |
| LP-18-a | LP-446-a |
| LP-18-a | LP-447-a |
| LP-18-a | LP-453-a |
| LP-18-a | LP-455-a |
| LP-18-a | LP-457-a |
| LP-18-a | LP-458-a |
| LP-18-a | LP-459-a |
| LP-18-a | LP-460-a |
| LP-18-a | LP-461-a |
| LP-18-a | LP-468-a |
| LP-18-a | LP-469-a |
| LP-18-a | LP-470-a |
| LP-18-a | LP-473-a |
| LP-18-a | LP-474-a |
| LP-18-a | CNR1 SM2-a |
| LP-18-a | (None) |
| LP-128-a | LP-4-a |
| LP-128-a | LP-18-a |
| LP-128-a | LP-128-a |
| LP-128-a | LP-151-a |
| LP-128-a | LP-183-a |
| LP-128-a | LP-200-a |
| LP-128-a | LP-208-a |
| LP-128-a | LP-211-a |
| LP-128-a | LP-232-a |
| LP-128-a | LP-242-a |
| LP-128-a | LP-243-a |
| LP-128-a | LP-244-a |
| LP-128-a | LP-245-a |
| LP-128-a | LP-249-a |
| LP-128-a | LP-274-a |
| LP-128-a | LP-295-a |
| LP-128-a | LP-310-a |
| LP-128-a | LP-359-a |
| LP-128-a | LP-361-a |
| LP-128-a | LP-371-a |
| LP-128-a | LP-374-a |
| LP-128-a | LP-375-a |
| LP-128-a | LP-377-a |
| LP-128-a | LP-378-a |
| LP-128-a | LP-379-a |
| LP-128-a | LP-380-a |
| LP-128-a | LP-403-a |
| LP-128-a | LP-404-a |
| LP-128-a | LP-412-a |
| LP-128-a | LP-413-a |
| LP-128-a | LP-424-a |
| LP-128-a | LP-425-a |
| LP-128-a | LP-426-a |
| LP-128-a | LP-427-a |
| LP-128-a | LP-428-a |
| LP-128-a | LP-432-a |
| LP-128-a | LP-433-a |
| LP-128-a | LP-444-a |
| LP-128-a | LP-445-a |
| LP-128-a | LP-446-a |
| LP-128-a | LP-447-a |
| LP-128-a | LP-453-a |
| LP-128-a | LP-455-a |
| LP-128-a | LP-457-a |
| LP-128-a | LP-458-a |
| LP-128-a | LP-459-a |
| LP-128-a | LP-460-a |
| LP-128-a | LP-461-a |
| LP-128-a | LP-468-a |
| LP-128-a | LP-469-a |
| LP-128-a | LP-470-a |
| LP-128-a | LP-473-a |
| LP-128-a | LP-474-a |
| LP-128-a | CNR1 SM2-a |
| LP-128-a | (None) |
| LP-151-a | LP-4-a |
| LP-151-a | LP-18-a |
| LP-151-a | LP-128-a |
| LP-151-a | LP-151-a |
| LP-151-a | LP-183-a |
| LP-151-a | LP-200-a |
| LP-151-a | LP-208-a |
| LP-151-a | LP-211-a |
| LP-151-a | LP-232-a |
| LP-151-a | LP-242-a |
| LP-151-a | LP-243-a |
| LP-151-a | LP-244-a |
| LP-151-a | LP-245-a |
| LP-151-a | LP-249-a |
| LP-151-a | LP-274-a |
| LP-151-a | LP-295-a |
| LP-151-a | LP-310-a |
| LP-151-a | LP-359-a |
| LP-151-a | LP-361-a |
| LP-151-a | LP-371-a |
| LP-151-a | LP-374-a |
| LP-151-a | LP-375-a |
| LP-151-a | LP-377-a |
| LP-151-a | LP-378-a |
| LP-151-a | LP-379-a |
| LP-151-a | LP-380-a |
| LP-151-a | LP-403-a |
| LP-151-a | LP-404-a |
| LP-151-a | LP-412-a |
| LP-151-a | LP-413-a |
| LP-151-a | LP-424-a |
| LP-151-a | LP-425-a |
| LP-151-a | LP-426-a |
| LP-151-a | LP-427-a |
| LP-151-a | LP-428-a |
| LP-151-a | LP-432-a |
| LP-151-a | LP-433-a |
| LP-151-a | LP-444-a |
| LP-151-a | LP-445-a |
| LP-151-a | LP-446-a |
| LP-151-a | LP-447-a |
| LP-151-a | LP-453-a |
| LP-151-a | LP-455-a |
| LP-151-a | LP-457-a |
| LP-151-a | LP-458-a |
| LP-151-a | LP-459-a |
| LP-151-a | LP-460-a |
| LP-151-a | LP-461-a |
| LP-151-a | LP-468-a |
| LP-151-a | LP-469-a |
| LP-151-a | LP-470-a |
| LP-151-a | LP-473-a |
| LP-151-a | LP-474-a |
| LP-151-a | CNR1 SM2-a |
| LP-151-a | (None) |
| LP-183-a | LP-4-a |
| LP-183-a | LP-18-a |
| LP-183-a | LP-128-a |
| LP-183-a | LP-151-a |
| LP-183-a | LP-183-a |
| LP-183-a | LP-200-a |
| LP-183-a | LP-208-a |
| LP-183-a | LP-211-a |
| LP-183-a | LP-232-a |
| LP-183-a | LP-242-a |
| LP-183-a | LP-243-a |
| LP-183-a | LP-244-a |
| LP-183-a | LP-245-a |
| LP-183-a | LP-249-a |
| LP-183-a | LP-274-a |
| LP-183-a | LP-295-a |
| LP-183-a | LP-310-a |
| LP-183-a | LP-359-a |
| LP-183-a | LP-361-a |
| LP-183-a | LP-371-a |
| LP-183-a | LP-374-a |
| LP-183-a | LP-375-a |
| LP-183-a | LP-377-a |
| LP-183-a | LP-378-a |
| LP-183-a | LP-379-a |
| LP-183-a | LP-380-a |
| LP-183-a | LP-403-a |
| LP-183-a | LP-404-a |
| LP-183-a | LP-412-a |
| LP-183-a | LP-413-a |
| LP-183-a | LP-424-a |
| LP-183-a | LP-425-a |
| LP-183-a | LP-426-a |
| LP-183-a | LP-427-a |
| LP-183-a | LP-428-a |
| LP-183-a | LP-432-a |
| LP-183-a | LP-433-a |
| LP-183-a | LP-444-a |
| LP-183-a | LP-445-a |
| LP-183-a | LP-446-a |
| LP-183-a | LP-447-a |
| LP-183-a | LP-453-a |
| LP-183-a | LP-455-a |
| LP-183-a | LP-457-a |
| LP-183-a | LP-458-a |
| LP-183-a | LP-459-a |
| LP-183-a | LP-460-a |
| LP-183-a | LP-461-a |
| LP-183-a | LP-468-a |
| LP-183-a | LP-469-a |
| LP-183-a | LP-470-a |
| LP-183-a | LP-473-a |
| LP-183-a | LP-474-a |
| LP-183-a | CNR1 SM2-a |
| LP-183-a | (None) |
| LP-200-a | LP-4-a |
| LP-200-a | LP-18-a |
| LP-200-a | LP-128-a |
| LP-200-a | LP-151-a |
| LP-200-a | LP-183-a |
| LP-200-a | LP-200-a |
| LP-200-a | LP-208-a |
| LP-200-a | LP-211-a |
| LP-200-a | LP-232-a |
| LP-200-a | LP-242-a |
| LP-200-a | LP-243-a |
| LP-200-a | LP-244-a |
| LP-200-a | LP-245-a |
| LP-200-a | LP-249-a |
| LP-200-a | LP-274-a |
| LP-200-a | LP-295-a |
| LP-200-a | LP-310-a |
| LP-200-a | LP-359-a |
| LP-200-a | LP-361-a |
| LP-200-a | LP-371-a |
| LP-200-a | LP-374-a |
| LP-200-a | LP-375-a |
| LP-200-a | LP-377-a |
| LP-200-a | LP-378-a |
| LP-200-a | LP-379-a |
| LP-200-a | LP-380-a |
| LP-200-a | LP-403-a |
| LP-200-a | LP-404-a |
| LP-200-a | LP-412-a |
| LP-200-a | LP-413-a |
| LP-200-a | LP-424-a |
| LP-200-a | LP-425-a |
| LP-200-a | LP-426-a |
| LP-200-a | LP-427-a |
| LP-200-a | LP-428-a |
| LP-200-a | LP-432-a |
| LP-200-a | LP-433-a |
| LP-200-a | LP-444-a |
| LP-200-a | LP-445-a |
| LP-200-a | LP-446-a |
| LP-200-a | LP-447-a |
| LP-200-a | LP-453-a |
| LP-200-a | LP-455-a |
| LP-200-a | LP-457-a |
| LP-200-a | LP-458-a |
| LP-200-a | LP-459-a |
| LP-200-a | LP-460-a |
| LP-200-a | LP-461-a |
| LP-200-a | LP-468-a |
| LP-200-a | LP-469-a |
| LP-200-a | LP-470-a |
| LP-200-a | LP-473-a |
| LP-200-a | LP-474-a |
| LP-200-a | CNR1 SM2-a |
| LP-200-a | (None) |
| LP-208-a | LP-4-a |
| LP-208-a | LP-18-a |
| LP-208-a | LP-128-a |
| LP-208-a | LP-151-a |
| LP-208-a | LP-183-a |
| LP-208-a | LP-200-a |
| LP-208-a | LP-208-a |
| LP-208-a | LP-211-a |
| LP-208-a | LP-232-a |
| LP-208-a | LP-242-a |
| LP-208-a | LP-243-a |
| LP-208-a | LP-244-a |
| LP-208-a | LP-245-a |
| LP-208-a | LP-249-a |
| LP-208-a | LP-274-a |
| LP-208-a | LP-295-a |
| LP-208-a | LP-310-a |
| LP-208-a | LP-359-a |
| LP-208-a | LP-361-a |
| LP-208-a | LP-371-a |
| LP-208-a | LP-374-a |
| LP-208-a | LP-375-a |
| LP-208-a | LP-377-a |
| LP-208-a | LP-378-a |
| LP-208-a | LP-379-a |
| LP-208-a | LP-380-a |
| LP-208-a | LP-403-a |
| LP-208-a | LP-404-a |
| LP-208-a | LP-412-a |
| LP-208-a | LP-413-a |
| LP-208-a | LP-424-a |
| LP-208-a | LP-425-a |
| LP-208-a | LP-426-a |
| LP-208-a | LP-427-a |
| LP-208-a | LP-428-a |
| LP-208-a | LP-432-a |
| LP-208-a | LP-433-a |
| LP-208-a | LP-444-a |
| LP-208-a | LP-445-a |
| LP-208-a | LP-446-a |
| LP-208-a | LP-447-a |
| LP-208-a | LP-453-a |
| LP-208-a | LP-455-a |
| LP-208-a | LP-457-a |
| LP-208-a | LP-458-a |
| LP-208-a | LP-459-a |
| LP-208-a | LP-460-a |
| LP-208-a | LP-461-a |
| LP-208-a | LP-468-a |
| LP-208-a | LP-469-a |
| LP-208-a | LP-470-a |
| LP-208-a | LP-473-a |
| LP-208-a | LP-474-a |
| LP-208-a | CNR1 SM2-a |
| LP-208-a | (None) |
| LP-211-a | LP-4-a |
| LP-211-a | LP-18-a |
| LP-211-a | LP-128-a |
| LP-211-a | LP-151-a |
| LP-211-a | LP-183-a |
| LP-211-a | LP-200-a |
| LP-211-a | LP-208-a |
| LP-211-a | LP-211-a |
| LP-211-a | LP-232-a |
| LP-211-a | LP-242-a |
| LP-211-a | LP-243-a |
| LP-211-a | LP-244-a |
| LP-211-a | LP-245-a |
| LP-211-a | LP-249-a |
| LP-211-a | LP-274-a |
| LP-211-a | LP-295-a |
| LP-211-a | LP-310-a |
| LP-211-a | LP-359-a |
| LP-211-a | LP-361-a |
| LP-211-a | LP-371-a |
| LP-211-a | LP-374-a |
| LP-211-a | LP-375-a |
| LP-211-a | LP-377-a |
| LP-211-a | LP-378-a |
| LP-211-a | LP-379-a |
| LP-211-a | LP-380-a |
| LP-211-a | LP-403-a |
| LP-211-a | LP-404-a |
| LP-211-a | LP-412-a |
| LP-211-a | LP-413-a |
| LP-211-a | LP-424-a |
| LP-211-a | LP-425-a |
| LP-211-a | LP-426-a |
| LP-211-a | LP-427-a |
| LP-211-a | LP-428-a |
| LP-211-a | LP-432-a |
| LP-211-a | LP-433-a |
| LP-211-a | LP-444-a |
| LP-211-a | LP-445-a |
| LP-211-a | LP-446-a |
| LP-211-a | LP-447-a |
| LP-211-a | LP-453-a |
| LP-211-a | LP-455-a |
| LP-211-a | LP-457-a |
| LP-211-a | LP-458-a |
| LP-211-a | LP-459-a |
| LP-211-a | LP-460-a |
| LP-211-a | LP-461-a |
| LP-211-a | LP-468-a |
| LP-211-a | LP-469-a |
| LP-211-a | LP-470-a |
| LP-211-a | LP-473-a |
| LP-211-a | LP-474-a |
| LP-211-a | CNR1 SM2-a |
| LP-211-a | (None) |
| LP-232-a | LP-4-a |
| LP-232-a | LP-18-a |
| LP-232-a | LP-128-a |
| LP-232-a | LP-151-a |
| LP-232-a | LP-183-a |
| LP-232-a | LP-200-a |
| LP-232-a | LP-208-a |
| LP-232-a | LP-211-a |
| LP-232-a | LP-232-a |
| LP-232-a | LP-242-a |
| LP-232-a | LP-243-a |
| LP-232-a | LP-244-a |
| LP-232-a | LP-245-a |
| LP-232-a | LP-249-a |
| LP-232-a | LP-274-a |
| LP-232-a | LP-295-a |
| LP-232-a | LP-310-a |
| LP-232-a | LP-359-a |
| LP-232-a | LP-361-a |
| LP-232-a | LP-371-a |
| LP-232-a | LP-374-a |
| LP-232-a | LP-375-a |
| LP-232-a | LP-377-a |
| LP-232-a | LP-378-a |
| LP-232-a | LP-379-a |
| LP-232-a | LP-380-a |
| LP-232-a | LP-403-a |
| LP-232-a | LP-404-a |
| LP-232-a | LP-412-a |
| LP-232-a | LP-413-a |
| LP-232-a | LP-424-a |
| LP-232-a | LP-425-a |
| LP-232-a | LP-426-a |
| LP-232-a | LP-427-a |
| LP-232-a | LP-428-a |
| LP-232-a | LP-432-a |
| LP-232-a | LP-433-a |
| LP-232-a | LP-444-a |
| LP-232-a | LP-445-a |
| LP-232-a | LP-446-a |
| LP-232-a | LP-447-a |
| LP-232-a | LP-453-a |
| LP-232-a | LP-455-a |
| LP-232-a | LP-457-a |
| LP-232-a | LP-458-a |
| LP-232-a | LP-459-a |
| LP-232-a | LP-460-a |
| LP-232-a | LP-461-a |
| LP-232-a | LP-468-a |
| LP-232-a | LP-469-a |
| LP-232-a | LP-470-a |
| LP-232-a | LP-473-a |
| LP-232-a | LP-474-a |
| LP-232-a | CNR1 SM2-a |
| LP-232-a | (None) |
| LP-242-a | LP-4-a |
| LP-242-a | LP-18-a |
| LP-242-a | LP-128-a |
| LP-242-a | LP-151-a |
| LP-242-a | LP-183-a |
| LP-242-a | LP-200-a |
| LP-242-a | LP-208-a |
| LP-242-a | LP-211-a |
| LP-242-a | LP-232-a |
| LP-242-a | LP-242-a |
| LP-242-a | LP-243-a |
| LP-242-a | LP-244-a |
| LP-242-a | LP-245-a |
| LP-242-a | LP-249-a |
| LP-242-a | LP-274-a |
| LP-242-a | LP-295-a |
| LP-242-a | LP-310-a |
| LP-242-a | LP-359-a |
| LP-242-a | LP-361-a |
| LP-242-a | LP-371-a |
| LP-242-a | LP-374-a |
| LP-242-a | LP-375-a |
| LP-242-a | LP-377-a |
| LP-242-a | LP-378-a |
| LP-242-a | LP-379-a |
| LP-242-a | LP-380-a |
| LP-242-a | LP-403-a |
| LP-242-a | LP-404-a |
| LP-242-a | LP-412-a |
| LP-242-a | LP-413-a |
| LP-242-a | LP-424-a |
| LP-242-a | LP-425-a |
| LP-242-a | LP-426-a |
| LP-242-a | LP-427-a |
| LP-242-a | LP-428-a |
| LP-242-a | LP-432-a |
| LP-242-a | LP-433-a |
| LP-242-a | LP-444-a |
| LP-242-a | LP-445-a |
| LP-242-a | LP-446-a |
| LP-242-a | LP-447-a |
| LP-242-a | LP-453-a |
| LP-242-a | LP-455-a |
| LP-242-a | LP-457-a |
| LP-242-a | LP-458-a |
| LP-242-a | LP-459-a |
| LP-242-a | LP-460-a |
| LP-242-a | LP-461-a |
| LP-242-a | LP-468-a |
| LP-242-a | LP-469-a |
| LP-242-a | LP-470-a |
| LP-242-a | LP-473-a |
| LP-242-a | LP-474-a |
| LP-242-a | CNR1 SM2-a |
| LP-242-a | (None) |
| LP-243-a | LP-4-a |
| LP-243-a | LP-18-a |
| LP-243-a | LP-128-a |
| LP-243-a | LP-151-a |
| LP-243-a | LP-183-a |
| LP-243-a | LP-200-a |
| LP-243-a | LP-208-a |
| LP-243-a | LP-211-a |
| LP-243-a | LP-232-a |
| LP-243-a | LP-242-a |
| LP-243-a | LP-243-a |
| LP-243-a | LP-244-a |
| LP-243-a | LP-245-a |
| LP-243-a | LP-249-a |
| LP-243-a | LP-274-a |
| LP-243-a | LP-295-a |
| LP-243-a | LP-310-a |
| LP-243-a | LP-359-a |
| LP-243-a | LP-361-a |
| LP-243-a | LP-371-a |
| LP-243-a | LP-374-a |
| LP-243-a | LP-375-a |
| LP-243-a | LP-377-a |
| LP-243-a | LP-378-a |
| LP-243-a | LP-379-a |
| LP-243-a | LP-380-a |
| LP-243-a | LP-403-a |
| LP-243-a | LP-404-a |
| LP-243-a | LP-412-a |
| LP-243-a | LP-413-a |
| LP-243-a | LP-424-a |
| LP-243-a | LP-425-a |
| LP-243-a | LP-426-a |
| LP-243-a | LP-427-a |
| LP-243-a | LP-428-a |
| LP-243-a | LP-432-a |
| LP-243-a | LP-433-a |
| LP-243-a | LP-444-a |
| LP-243-a | LP-445-a |
| LP-243-a | LP-446-a |
| LP-243-a | LP-447-a |
| LP-243-a | LP-453-a |
| LP-243-a | LP-455-a |
| LP-243-a | LP-457-a |
| LP-243-a | LP-458-a |
| LP-243-a | LP-459-a |
| LP-243-a | LP-460-a |
| LP-243-a | LP-461-a |
| LP-243-a | LP-468-a |
| LP-243-a | LP-469-a |
| LP-243-a | LP-470-a |
| LP-243-a | LP-473-a |
| LP-243-a | LP-474-a |
| LP-243-a | CNR1 SM2-a |
| LP-243-a | (None) |
| LP-244-a | LP-4-a |
| LP-244-a | LP-18-a |
| LP-244-a | LP-128-a |
| LP-244-a | LP-151-a |
| LP-244-a | LP-183-a |
| LP-244-a | LP-200-a |
| LP-244-a | LP-208-a |
| LP-244-a | LP-211-a |
| LP-244-a | LP-232-a |
| LP-244-a | LP-242-a |
| LP-244-a | LP-243-a |
| LP-244-a | LP-244-a |
| LP-244-a | LP-245-a |
| LP-244-a | LP-249-a |
| LP-244-a | LP-274-a |
| LP-244-a | LP-295-a |
| LP-244-a | LP-310-a |
| LP-244-a | LP-359-a |
| LP-244-a | LP-361-a |
| LP-244-a | LP-371-a |
| LP-244-a | LP-374-a |
| LP-244-a | LP-375-a |
| LP-244-a | LP-377-a |
| LP-244-a | LP-378-a |
| LP-244-a | LP-379-a |
| LP-244-a | LP-380-a |
| LP-244-a | LP-403-a |
| LP-244-a | LP-404-a |
| LP-244-a | LP-412-a |
| LP-244-a | LP-413-a |
| LP-244-a | LP-424-a |
| LP-244-a | LP-425-a |
| LP-244-a | LP-426-a |
| LP-244-a | LP-427-a |
| LP-244-a | LP-428-a |
| LP-244-a | LP-432-a |
| LP-244-a | LP-433-a |
| LP-244-a | LP-444-a |
| LP-244-a | LP-445-a |
| LP-244-a | LP-446-a |
| LP-244-a | LP-447-a |
| LP-244-a | LP-453-a |
| LP-244-a | LP-455-a |
| LP-244-a | LP-457-a |
| LP-244-a | LP-458-a |
| LP-244-a | LP-459-a |
| LP-244-a | LP-460-a |
| LP-244-a | LP-461-a |
| LP-244-a | LP-468-a |
| LP-244-a | LP-469-a |
| LP-244-a | LP-470-a |
| LP-244-a | LP-473-a |
| LP-244-a | LP-474-a |
| LP-244-a | CNR1 SM2-a |
| LP-244-a | (None) |
| LP-245-a | LP-4-a |
| LP-245-a | LP-18-a |
| LP-245-a | LP-128-a |
| LP-245-a | LP-151-a |
| LP-245-a | LP-183-a |
| LP-245-a | LP-200-a |
| LP-245-a | LP-208-a |
| LP-245-a | LP-211-a |
| LP-245-a | LP-232-a |
| LP-245-a | LP-242-a |
| LP-245-a | LP-243-a |
| LP-245-a | LP-244-a |
| LP-245-a | LP-245-a |
| LP-245-a | LP-249-a |
| LP-245-a | LP-274-a |
| LP-245-a | LP-295-a |
| LP-245-a | LP-310-a |
| LP-245-a | LP-359-a |
| LP-245-a | LP-361-a |
| LP-245-a | LP-371-a |
| LP-245-a | LP-374-a |
| LP-245-a | LP-375-a |
| LP-245-a | LP-377-a |
| LP-245-a | LP-378-a |
| LP-245-a | LP-379-a |
| LP-245-a | LP-380-a |
| LP-245-a | LP-403-a |
| LP-245-a | LP-404-a |
| LP-245-a | LP-412-a |
| LP-245-a | LP-413-a |
| LP-245-a | LP-424-a |
| LP-245-a | LP-425-a |
| LP-245-a | LP-426-a |
| LP-245-a | LP-427-a |
| LP-245-a | LP-428-a |
| LP-245-a | LP-432-a |
| LP-245-a | LP-433-a |
| LP-245-a | LP-444-a |
| LP-245-a | LP-445-a |
| LP-245-a | LP-446-a |
| LP-245-a | LP-447-a |
| LP-245-a | LP-453-a |
| LP-245-a | LP-455-a |
| LP-245-a | LP-457-a |
| LP-245-a | LP-458-a |
| LP-245-a | LP-459-a |
| LP-245-a | LP-460-a |
| LP-245-a | LP-461-a |
| LP-245-a | LP-468-a |
| LP-245-a | LP-469-a |
| LP-245-a | LP-470-a |
| LP-245-a | LP-473-a |
| LP-245-a | LP-474-a |
| LP-245-a | CNR1 SM2-a |
| LP-245-a | (None) |
| LP-249-a | LP-4-a |
| LP-249-a | LP-18-a |
| LP-249-a | LP-128-a |
| LP-249-a | LP-151-a |
| LP-249-a | LP-183-a |
| LP-249-a | LP-200-a |
| LP-249-a | LP-208-a |
| LP-249-a | LP-211-a |
| LP-249-a | LP-232-a |
| LP-249-a | LP-242-a |
| LP-249-a | LP-243-a |
| LP-249-a | LP-244-a |
| LP-249-a | LP-245-a |
| LP-249-a | LP-249-a |
| LP-249-a | LP-274-a |
| LP-249-a | LP-295-a |
| LP-249-a | LP-310-a |
| LP-249-a | LP-359-a |
| LP-249-a | LP-361-a |
| LP-249-a | LP-371-a |
| LP-249-a | LP-374-a |
| LP-249-a | LP-375-a |
| LP-249-a | LP-377-a |
| LP-249-a | LP-378-a |
| LP-249-a | LP-379-a |
| LP-249-a | LP-380-a |
| LP-249-a | LP-403-a |
| LP-249-a | LP-404-a |
| LP-249-a | LP-412-a |
| LP-249-a | LP-413-a |
| LP-249-a | LP-424-a |
| LP-249-a | LP-425-a |
| LP-249-a | LP-426-a |
| LP-249-a | LP-427-a |
| LP-249-a | LP-428-a |
| LP-249-a | LP-432-a |
| LP-249-a | LP-433-a |
| LP-249-a | LP-444-a |
| LP-249-a | LP-445-a |
| LP-249-a | LP-446-a |
| LP-249-a | LP-447-a |
| LP-249-a | LP-453-a |
| LP-249-a | LP-455-a |
| LP-249-a | LP-457-a |
| LP-249-a | LP-458-a |
| LP-249-a | LP-459-a |
| LP-249-a | LP-460-a |
| LP-249-a | LP-461-a |
| LP-249-a | LP-468-a |
| LP-249-a | LP-469-a |
| LP-249-a | LP-470-a |
| LP-249-a | LP-473-a |
| LP-249-a | LP-474-a |
| LP-249-a | CNR1 SM2-a |
| LP-249-a | (None) |
| LP-274-a | LP-4-a |
| LP-274-a | LP-18-a |
| LP-274-a | LP-128-a |
| LP-274-a | LP-151-a |
| LP-274-a | LP-183-a |
| LP-274-a | LP-200-a |
| LP-274-a | LP-208-a |
| LP-274-a | LP-211-a |
| LP-274-a | LP-232-a |
| LP-274-a | LP-242-a |
| LP-274-a | LP-243-a |
| LP-274-a | LP-244-a |
| LP-274-a | LP-245-a |
| LP-274-a | LP-249-a |
| LP-274-a | LP-274-a |
| LP-274-a | LP-295-a |
| LP-274-a | LP-310-a |
| LP-274-a | LP-359-a |
| LP-274-a | LP-361-a |
| LP-274-a | LP-371-a |
| LP-274-a | LP-374-a |
| LP-274-a | LP-375-a |
| LP-274-a | LP-377-a |
| LP-274-a | LP-378-a |
| LP-274-a | LP-379-a |
| LP-274-a | LP-380-a |
| LP-274-a | LP-403-a |
| LP-274-a | LP-404-a |
| LP-274-a | LP-412-a |
| LP-274-a | LP-413-a |
| LP-274-a | LP-424-a |
| LP-274-a | LP-425-a |
| LP-274-a | LP-426-a |
| LP-274-a | LP-427-a |
| LP-274-a | LP-428-a |
| LP-274-a | LP-432-a |
| LP-274-a | LP-433-a |
| LP-274-a | LP-444-a |
| LP-274-a | LP-445-a |
| LP-274-a | LP-446-a |
| LP-274-a | LP-447-a |
| LP-274-a | LP-453-a |
| LP-274-a | LP-455-a |
| LP-274-a | LP-457-a |
| LP-274-a | LP-458-a |
| LP-274-a | LP-459-a |
| LP-274-a | LP-460-a |
| LP-274-a | LP-461-a |
| LP-274-a | LP-468-a |
| LP-274-a | LP-469-a |
| LP-274-a | LP-470-a |
| LP-274-a | LP-473-a |
| LP-274-a | LP-474-a |
| LP-274-a | CNR1 SM2-a |
| LP-274-a | (None) |
| LP-295-a | LP-4-a |
| LP-295-a | LP-18-a |
| LP-295-a | LP-128-a |
| LP-295-a | LP-151-a |
| LP-295-a | LP-183-a |
| LP-295-a | LP-200-a |
| LP-295-a | LP-208-a |
| LP-295-a | LP-211-a |
| LP-295-a | LP-232-a |
| LP-295-a | LP-242-a |
| LP-295-a | LP-243-a |
| LP-295-a | LP-244-a |
| LP-295-a | LP-245-a |
| LP-295-a | LP-249-a |
| LP-295-a | LP-274-a |
| LP-295-a | LP-295-a |
| LP-295-a | LP-310-a |
| LP-295-a | LP-359-a |
| LP-295-a | LP-361-a |
| LP-295-a | LP-371-a |
| LP-295-a | LP-374-a |
| LP-295-a | LP-375-a |
| LP-295-a | LP-377-a |
| LP-295-a | LP-378-a |
| LP-295-a | LP-379-a |
| LP-295-a | LP-380-a |
| LP-295-a | LP-403-a |
| LP-295-a | LP-404-a |
| LP-295-a | LP-412-a |
| LP-295-a | LP-413-a |
| LP-295-a | LP-424-a |
| LP-295-a | LP-425-a |
| LP-295-a | LP-426-a |
| LP-295-a | LP-427-a |
| LP-295-a | LP-428-a |
| LP-295-a | LP-432-a |
| LP-295-a | LP-433-a |
| LP-295-a | LP-444-a |
| LP-295-a | LP-445-a |
| LP-295-a | LP-446-a |
| LP-295-a | LP-447-a |
| LP-295-a | LP-453-a |
| LP-295-a | LP-455-a |
| LP-295-a | LP-457-a |
| LP-295-a | LP-458-a |
| LP-295-a | LP-459-a |
| LP-295-a | LP-460-a |
| LP-295-a | LP-461-a |
| LP-295-a | LP-468-a |
| LP-295-a | LP-469-a |
| LP-295-a | LP-470-a |
| LP-295-a | LP-473-a |
| LP-295-a | LP-474-a |
| LP-295-a | CNR1 SM2-a |
| LP-295-a | (None) |
| LP-310-a | LP-4-a |
| LP-310-a | LP-18-a |
| LP-310-a | LP-128-a |
| LP-310-a | LP-151-a |
| LP-310-a | LP-183-a |
| LP-310-a | LP-200-a |
| LP-310-a | LP-208-a |
| LP-310-a | LP-211-a |
| LP-310-a | LP-232-a |
| LP-310-a | LP-242-a |
| LP-310-a | LP-243-a |
| LP-310-a | LP-244-a |
| LP-310-a | LP-245-a |
| LP-310-a | LP-249-a |
| LP-310-a | LP-274-a |
| LP-310-a | LP-295-a |
| LP-310-a | LP-310-a |
| LP-310-a | LP-359-a |
| LP-310-a | LP-361-a |
| LP-310-a | LP-371-a |
| LP-310-a | LP-374-a |
| LP-310-a | LP-375-a |
| LP-310-a | LP-377-a |
| LP-310-a | LP-378-a |
| LP-310-a | LP-379-a |
| LP-310-a | LP-380-a |
| LP-310-a | LP-403-a |
| LP-310-a | LP-404-a |
| LP-310-a | LP-412-a |
| LP-310-a | LP-413-a |
| LP-310-a | LP-424-a |
| LP-310-a | LP-425-a |
| LP-310-a | LP-426-a |
| LP-310-a | LP-427-a |
| LP-310-a | LP-428-a |
| LP-310-a | LP-432-a |
| LP-310-a | LP-433-a |
| LP-310-a | LP-444-a |
| LP-310-a | LP-445-a |
| LP-310-a | LP-446-a |
| LP-310-a | LP-447-a |
| LP-310-a | LP-453-a |
| LP-310-a | LP-455-a |
| LP-310-a | LP-457-a |
| LP-310-a | LP-458-a |
| LP-310-a | LP-459-a |
| LP-310-a | LP-460-a |
| LP-310-a | LP-461-a |
| LP-310-a | LP-468-a |
| LP-310-a | LP-469-a |
| LP-310-a | LP-470-a |
| LP-310-a | LP-473-a |
| LP-310-a | LP-474-a |
| LP-310-a | CNR1 SM2-a |
| LP-310-a | (None) |
| LP-359-a | LP-4-a |
| LP-359-a | LP-18-a |
| LP-359-a | LP-128-a |
| LP-359-a | LP-151-a |
| LP-359-a | LP-183-a |
| LP-359-a | LP-200-a |
| LP-359-a | LP-208-a |
| LP-359-a | LP-211-a |
| LP-359-a | LP-232-a |
| LP-359-a | LP-242-a |
| LP-359-a | LP-243-a |
| LP-359-a | LP-244-a |
| LP-359-a | LP-245-a |
| LP-359-a | LP-249-a |
| LP-359-a | LP-274-a |
| LP-359-a | LP-295-a |
| LP-359-a | LP-310-a |
| LP-359-a | LP-359-a |
| LP-359-a | LP-361-a |
| LP-359-a | LP-371-a |
| LP-359-a | LP-374-a |
| LP-359-a | LP-375-a |
| LP-359-a | LP-377-a |
| LP-359-a | LP-378-a |
| LP-359-a | LP-379-a |
| LP-359-a | LP-380-a |
| LP-359-a | LP-403-a |
| LP-359-a | LP-404-a |
| LP-359-a | LP-412-a |
| LP-359-a | LP-413-a |
| LP-359-a | LP-424-a |
| LP-359-a | LP-425-a |
| LP-359-a | LP-426-a |
| LP-359-a | LP-427-a |
| LP-359-a | LP-428-a |
| LP-359-a | LP-432-a |
| LP-359-a | LP-433-a |
| LP-359-a | LP-444-a |
| LP-359-a | LP-445-a |
| LP-359-a | LP-446-a |
| LP-359-a | LP-447-a |
| LP-359-a | LP-453-a |
| LP-359-a | LP-455-a |
| LP-359-a | LP-457-a |
| LP-359-a | LP-458-a |
| LP-359-a | LP-459-a |
| LP-359-a | LP-460-a |
| LP-359-a | LP-461-a |
| LP-359-a | LP-468-a |
| LP-359-a | LP-469-a |
| LP-359-a | LP-470-a |
| LP-359-a | LP-473-a |
| LP-359-a | LP-474-a |
| LP-359-a | CNR1 SM2-a |
| LP-359-a | (None) |
| LP-361-a | LP-4-a |
| LP-361-a | LP-18-a |
| LP-361-a | LP-128-a |
| LP-361-a | LP-151-a |
| LP-361-a | LP-183-a |
| LP-361-a | LP-200-a |
| LP-361-a | LP-208-a |
| LP-361-a | LP-211-a |
| LP-361-a | LP-232-a |
| LP-361-a | LP-242-a |
| LP-361-a | LP-243-a |
| LP-361-a | LP-244-a |
| LP-361-a | LP-245-a |
| LP-361-a | LP-249-a |
| LP-361-a | LP-274-a |
| LP-361-a | LP-295-a |
| LP-361-a | LP-310-a |
| LP-361-a | LP-359-a |
| LP-361-a | LP-361-a |
| LP-361-a | LP-371-a |
| LP-361-a | LP-374-a |
| LP-361-a | LP-375-a |
| LP-361-a | LP-377-a |
| LP-361-a | LP-378-a |
| LP-361-a | LP-379-a |
| LP-361-a | LP-380-a |
| LP-361-a | LP-403-a |
| LP-361-a | LP-404-a |
| LP-361-a | LP-412-a |
| LP-361-a | LP-413-a |
| LP-361-a | LP-424-a |
| LP-361-a | LP-425-a |
| LP-361-a | LP-426-a |
| LP-361-a | LP-427-a |
| LP-361-a | LP-428-a |
| LP-361-a | LP-432-a |
| LP-361-a | LP-433-a |
| LP-361-a | LP-444-a |
| LP-361-a | LP-445-a |
| LP-361-a | LP-446-a |
| LP-361-a | LP-447-a |
| LP-361-a | LP-453-a |
| LP-361-a | LP-455-a |
| LP-361-a | LP-457-a |
| LP-361-a | LP-458-a |
| LP-361-a | LP-459-a |
| LP-361-a | LP-460-a |
| LP-361-a | LP-461-a |
| LP-361-a | LP-468-a |
| LP-361-a | LP-469-a |
| LP-361-a | LP-470-a |
| LP-361-a | LP-473-a |
| LP-361-a | LP-474-a |
| LP-361-a | CNR1 SM2-a |
| LP-361-a | (None) |
| LP-371-a | LP-4-a |
| LP-371-a | LP-18-a |
| LP-371-a | LP-128-a |
| LP-371-a | LP-151-a |
| LP-371-a | LP-183-a |
| LP-371-a | LP-200-a |
| LP-371-a | LP-208-a |
| LP-371-a | LP-211-a |
| LP-371-a | LP-232-a |
| LP-371-a | LP-242-a |
| LP-371-a | LP-243-a |
| LP-371-a | LP-244-a |
| LP-371-a | LP-245-a |
| LP-371-a | LP-249-a |
| LP-371-a | LP-274-a |
| LP-371-a | LP-295-a |
| LP-371-a | LP-310-a |
| LP-371-a | LP-359-a |
| LP-371-a | LP-361-a |
| LP-371-a | LP-371-a |
| LP-371-a | LP-374-a |
| LP-371-a | LP-375-a |
| LP-371-a | LP-377-a |
| LP-371-a | LP-378-a |
| LP-371-a | LP-379-a |
| LP-371-a | LP-380-a |
| LP-371-a | LP-403-a |
| LP-371-a | LP-404-a |
| LP-371-a | LP-412-a |
| LP-371-a | LP-413-a |
| LP-371-a | LP-424-a |
| LP-371-a | LP-425-a |
| LP-371-a | LP-426-a |
| LP-371-a | LP-427-a |
| LP-371-a | LP-428-a |
| LP-371-a | LP-432-a |
| LP-371-a | LP-433-a |
| LP-371-a | LP-444-a |
| LP-371-a | LP-445-a |
| LP-371-a | LP-446-a |
| LP-371-a | LP-447-a |
| LP-371-a | LP-453-a |
| LP-371-a | LP-455-a |
| LP-371-a | LP-457-a |
| LP-371-a | LP-458-a |
| LP-371-a | LP-459-a |
| LP-371-a | LP-460-a |
| LP-371-a | LP-461-a |
| LP-371-a | LP-468-a |
| LP-371-a | LP-469-a |
| LP-371-a | LP-470-a |
| LP-371-a | LP-473-a |
| LP-371-a | LP-474-a |
| LP-371-a | CNR1 SM2-a |
| LP-371-a | (None) |
| LP-374-a | LP-4-a |
| LP-374-a | LP-18-a |
| LP-374-a | LP-128-a |
| LP-374-a | LP-151-a |
| LP-374-a | LP-183-a |
| LP-374-a | LP-200-a |
| LP-374-a | LP-208-a |
| LP-374-a | LP-211-a |
| LP-374-a | LP-232-a |
| LP-374-a | LP-242-a |
| LP-374-a | LP-243-a |
| LP-374-a | LP-244-a |
| LP-374-a | LP-245-a |
| LP-374-a | LP-249-a |
| LP-374-a | LP-274-a |
| LP-374-a | LP-295-a |
| LP-374-a | LP-310-a |
| LP-374-a | LP-359-a |
| LP-374-a | LP-361-a |
| LP-374-a | LP-371-a |
| LP-374-a | LP-374-a |
| LP-374-a | LP-375-a |
| LP-374-a | LP-377-a |
| LP-374-a | LP-378-a |
| LP-374-a | LP-379-a |
| LP-374-a | LP-380-a |
| LP-374-a | LP-403-a |
| LP-374-a | LP-404-a |
| LP-374-a | LP-412-a |
| LP-374-a | LP-413-a |
| LP-374-a | LP-424-a |
| LP-374-a | LP-425-a |
| LP-374-a | LP-426-a |
| LP-374-a | LP-427-a |
| LP-374-a | LP-428-a |
| LP-374-a | LP-432-a |
| LP-374-a | LP-433-a |
| LP-374-a | LP-444-a |
| LP-374-a | LP-445-a |
| LP-374-a | LP-446-a |
| LP-374-a | LP-447-a |
| LP-374-a | LP-453-a |
| LP-374-a | LP-455-a |
| LP-374-a | LP-457-a |
| LP-374-a | LP-458-a |
| LP-374-a | LP-459-a |
| LP-374-a | LP-460-a |
| LP-374-a | LP-461-a |
| LP-374-a | LP-468-a |
| LP-374-a | LP-469-a |
| LP-374-a | LP-470-a |
| LP-374-a | LP-473-a |
| LP-374-a | LP-474-a |
| LP-374-a | CNR1 SM2-a |
| LP-374-a | (None) |
| LP-375-a | LP-4-a |
| LP-375-a | LP-18-a |
| LP-375-a | LP-128-a |
| LP-375-a | LP-151-a |
| LP-375-a | LP-183-a |
| LP-375-a | LP-200-a |
| LP-375-a | LP-208-a |
| LP-375-a | LP-211-a |
| LP-375-a | LP-232-a |
| LP-375-a | LP-242-a |
| LP-375-a | LP-243-a |
| LP-375-a | LP-244-a |
| LP-375-a | LP-245-a |
| LP-375-a | LP-249-a |
| LP-375-a | LP-274-a |
| LP-375-a | LP-295-a |
| LP-375-a | LP-310-a |
| LP-375-a | LP-359-a |
| LP-375-a | LP-361-a |
| LP-375-a | LP-371-a |
| LP-375-a | LP-374-a |
| LP-375-a | LP-375-a |
| LP-375-a | LP-377-a |
| LP-375-a | LP-378-a |
| LP-375-a | LP-379-a |
| LP-375-a | LP-380-a |
| LP-375-a | LP-403-a |
| LP-375-a | LP-404-a |
| LP-375-a | LP-412-a |
| LP-375-a | LP-413-a |
| LP-375-a | LP-424-a |
| LP-375-a | LP-425-a |
| LP-375-a | LP-426-a |
| LP-375-a | LP-427-a |
| LP-375-a | LP-428-a |
| LP-375-a | LP-432-a |
| LP-375-a | LP-433-a |
| LP-375-a | LP-444-a |
| LP-375-a | LP-445-a |
| LP-375-a | LP-446-a |
| LP-375-a | LP-447-a |
| LP-375-a | LP-453-a |
| LP-375-a | LP-455-a |
| LP-375-a | LP-457-a |
| LP-375-a | LP-458-a |
| LP-375-a | LP-459-a |
| LP-375-a | LP-460-a |
| LP-375-a | LP-461-a |
| LP-375-a | LP-468-a |
| LP-375-a | LP-469-a |
| LP-375-a | LP-470-a |
| LP-375-a | LP-473-a |
| LP-375-a | LP-474-a |
| LP-375-a | CNR1 SM2-a |
| LP-375-a | (None) |
| LP-377-a | LP-4-a |
| LP-377-a | LP-18-a |
| LP-377-a | LP-128-a |
| LP-377-a | LP-151-a |
| LP-377-a | LP-183-a |
| LP-377-a | LP-200-a |
| LP-377-a | LP-208-a |
| LP-377-a | LP-211-a |
| LP-377-a | LP-232-a |
| LP-377-a | LP-242-a |
| LP-377-a | LP-243-a |
| LP-377-a | LP-244-a |
| LP-377-a | LP-245-a |
| LP-377-a | LP-249-a |
| LP-377-a | LP-274-a |
| LP-377-a | LP-295-a |
| LP-377-a | LP-310-a |
| LP-377-a | LP-359-a |
| LP-377-a | LP-361-a |
| LP-377-a | LP-371-a |
| LP-377-a | LP-374-a |
| LP-377-a | LP-375-a |
| LP-377-a | LP-377-a |
| LP-377-a | LP-378-a |
| LP-377-a | LP-379-a |
| LP-377-a | LP-380-a |
| LP-377-a | LP-403-a |
| LP-377-a | LP-404-a |
| LP-377-a | LP-412-a |
| LP-377-a | LP-413-a |
| LP-377-a | LP-424-a |
| LP-377-a | LP-425-a |
| LP-377-a | LP-426-a |
| LP-377-a | LP-427-a |
| LP-377-a | LP-428-a |
| LP-377-a | LP-432-a |
| LP-377-a | LP-433-a |
| LP-377-a | LP-444-a |
| LP-377-a | LP-445-a |
| LP-377-a | LP-446-a |
| LP-377-a | LP-447-a |
| LP-377-a | LP-453-a |
| LP-377-a | LP-455-a |
| LP-377-a | LP-457-a |
| LP-377-a | LP-458-a |
| LP-377-a | LP-459-a |
| LP-377-a | LP-460-a |
| LP-377-a | LP-461-a |
| LP-377-a | LP-468-a |
| LP-377-a | LP-469-a |
| LP-377-a | LP-470-a |
| LP-377-a | LP-473-a |
| LP-377-a | LP-474-a |
| LP-377-a | CNR1 SM2-a |
| LP-377-a | (None) |
| LP-378-a | LP-4-a |
| LP-378-a | LP-18-a |
| LP-378-a | LP-128-a |
| LP-378-a | LP-151-a |
| LP-378-a | LP-183-a |
| LP-378-a | LP-200-a |
| LP-378-a | LP-208-a |
| LP-378-a | LP-211-a |
| LP-378-a | LP-232-a |
| LP-378-a | LP-242-a |
| LP-378-a | LP-243-a |
| LP-378-a | LP-244-a |
| LP-378-a | LP-245-a |
| LP-378-a | LP-249-a |
| LP-378-a | LP-274-a |
| LP-378-a | LP-295-a |
| LP-378-a | LP-310-a |
| LP-378-a | LP-359-a |
| LP-378-a | LP-361-a |
| LP-378-a | LP-371-a |
| LP-378-a | LP-374-a |
| LP-378-a | LP-375-a |
| LP-378-a | LP-377-a |
| LP-378-a | LP-378-a |
| LP-378-a | LP-379-a |
| LP-378-a | LP-380-a |
| LP-378-a | LP-403-a |
| LP-378-a | LP-404-a |
| LP-378-a | LP-412-a |
| LP-378-a | LP-413-a |
| LP-378-a | LP-424-a |
| LP-378-a | LP-425-a |
| LP-378-a | LP-426-a |
| LP-378-a | LP-427-a |
| LP-378-a | LP-428-a |
| LP-378-a | LP-432-a |
| LP-378-a | LP-433-a |
| LP-378-a | LP-444-a |
| LP-378-a | LP-445-a |
| LP-378-a | LP-446-a |
| LP-378-a | LP-447-a |
| LP-378-a | LP-453-a |
| LP-378-a | LP-455-a |
| LP-378-a | LP-457-a |
| LP-378-a | LP-458-a |
| LP-378-a | LP-459-a |
| LP-378-a | LP-460-a |
| LP-378-a | LP-461-a |
| LP-378-a | LP-468-a |
| LP-378-a | LP-469-a |
| LP-378-a | LP-470-a |
| LP-378-a | LP-473-a |
| LP-378-a | LP-474-a |
| LP-378-a | CNR1 SM2-a |
| LP-378-a | (None) |
| LP-379-a | LP-4-a |
| LP-379-a | LP-18-a |
| LP-379-a | LP-128-a |
| LP-379-a | LP-151-a |
| LP-379-a | LP-183-a |
| LP-379-a | LP-200-a |
| LP-379-a | LP-208-a |
| LP-379-a | LP-211-a |
| LP-379-a | LP-232-a |
| LP-379-a | LP-242-a |
| LP-379-a | LP-243-a |
| LP-379-a | LP-244-a |
| LP-379-a | LP-245-a |
| LP-379-a | LP-249-a |
| LP-379-a | LP-274-a |
| LP-379-a | LP-295-a |
| LP-379-a | LP-310-a |
| LP-379-a | LP-359-a |
| LP-379-a | LP-361-a |
| LP-379-a | LP-371-a |
| LP-379-a | LP-374-a |
| LP-379-a | LP-375-a |
| LP-379-a | LP-377-a |
| LP-379-a | LP-378-a |
| LP-379-a | LP-379-a |
| LP-379-a | LP-380-a |
| LP-379-a | LP-403-a |
| LP-379-a | LP-404-a |
| LP-379-a | LP-412-a |
| LP-379-a | LP-413-a |
| LP-379-a | LP-424-a |
| LP-379-a | LP-425-a |
| LP-379-a | LP-426-a |
| LP-379-a | LP-427-a |
| LP-379-a | LP-428-a |
| LP-379-a | LP-432-a |
| LP-379-a | LP-433-a |
| LP-379-a | LP-444-a |
| LP-379-a | LP-445-a |
| LP-379-a | LP-446-a |
| LP-379-a | LP-447-a |
| LP-379-a | LP-453-a |
| LP-379-a | LP-455-a |
| LP-379-a | LP-457-a |
| LP-379-a | LP-458-a |
| LP-379-a | LP-459-a |
| LP-379-a | LP-460-a |
| LP-379-a | LP-461-a |
| LP-379-a | LP-468-a |
| LP-379-a | LP-469-a |
| LP-379-a | LP-470-a |
| LP-379-a | LP-473-a |
| LP-379-a | LP-474-a |
| LP-379-a | CNR1 SM2-a |
| LP-379-a | (None) |
| LP-380-a | LP-4-a |
| LP-380-a | LP-18-a |
| LP-380-a | LP-128-a |
| LP-380-a | LP-151-a |
| LP-380-a | LP-183-a |
| LP-380-a | LP-200-a |
| LP-380-a | LP-208-a |
| LP-380-a | LP-211-a |
| LP-380-a | LP-232-a |
| LP-380-a | LP-242-a |
| LP-380-a | LP-243-a |
| LP-380-a | LP-244-a |
| LP-380-a | LP-245-a |
| LP-380-a | LP-249-a |
| LP-380-a | LP-274-a |
| LP-380-a | LP-295-a |
| LP-380-a | LP-310-a |
| LP-380-a | LP-359-a |
| LP-380-a | LP-361-a |
| LP-380-a | LP-371-a |
| LP-380-a | LP-374-a |
| LP-380-a | LP-375-a |
| LP-380-a | LP-377-a |
| LP-380-a | LP-378-a |
| LP-380-a | LP-379-a |
| LP-380-a | LP-380-a |
| LP-380-a | LP-403-a |
| LP-380-a | LP-404-a |
| LP-380-a | LP-412-a |
| LP-380-a | LP-413-a |
| LP-380-a | LP-424-a |
| LP-380-a | LP-425-a |
| LP-380-a | LP-426-a |
| LP-380-a | LP-427-a |
| LP-380-a | LP-428-a |
| LP-380-a | LP-432-a |
| LP-380-a | LP-433-a |
| LP-380-a | LP-444-a |
| LP-380-a | LP-445-a |
| LP-380-a | LP-446-a |
| LP-380-a | LP-447-a |
| LP-380-a | LP-453-a |
| LP-380-a | LP-455-a |
| LP-380-a | LP-457-a |
| LP-380-a | LP-458-a |
| LP-380-a | LP-459-a |
| LP-380-a | LP-460-a |
| LP-380-a | LP-461-a |
| LP-380-a | LP-468-a |
| LP-380-a | LP-469-a |
| LP-380-a | LP-470-a |
| LP-380-a | LP-473-a |
| LP-380-a | LP-474-a |
| LP-380-a | CNR1 SM2-a |
| LP-380-a | (None) |
| LP-403-a | LP-4-a |
| LP-403-a | LP-18-a |
| LP-403-a | LP-128-a |
| LP-403-a | LP-151-a |
| LP-403-a | LP-183-a |
| LP-403-a | LP-200-a |
| LP-403-a | LP-208-a |
| LP-403-a | LP-211-a |
| LP-403-a | LP-232-a |
| LP-403-a | LP-242-a |
| LP-403-a | LP-243-a |
| LP-403-a | LP-244-a |
| LP-403-a | LP-245-a |
| LP-403-a | LP-249-a |
| LP-403-a | LP-274-a |
| LP-403-a | LP-295-a |
| LP-403-a | LP-310-a |
| LP-403-a | LP-359-a |
| LP-403-a | LP-361-a |
| LP-403-a | LP-371-a |
| LP-403-a | LP-374-a |
| LP-403-a | LP-375-a |
| LP-403-a | LP-377-a |
| LP-403-a | LP-378-a |
| LP-403-a | LP-379-a |
| LP-403-a | LP-380-a |
| LP-403-a | LP-403-a |
| LP-403-a | LP-404-a |
| LP-403-a | LP-412-a |
| LP-403-a | LP-413-a |
| LP-403-a | LP-424-a |
| LP-403-a | LP-425-a |
| LP-403-a | LP-426-a |
| LP-403-a | LP-427-a |
| LP-403-a | LP-428-a |
| LP-403-a | LP-432-a |
| LP-403-a | LP-433-a |
| LP-403-a | LP-444-a |
| LP-403-a | LP-445-a |
| LP-403-a | LP-446-a |
| LP-403-a | LP-447-a |
| LP-403-a | LP-453-a |
| LP-403-a | LP-455-a |
| LP-403-a | LP-457-a |
| LP-403-a | LP-458-a |
| LP-403-a | LP-459-a |
| LP-403-a | LP-460-a |
| LP-403-a | LP-461-a |
| LP-403-a | LP-468-a |
| LP-403-a | LP-469-a |
| LP-403-a | LP-470-a |
| LP-403-a | LP-473-a |
| LP-403-a | LP-474-a |
| LP-403-a | CNR1 SM2-a |
| LP-403-a | (None) |
| LP-404-a | LP-4-a |
| LP-404-a | LP-18-a |
| LP-404-a | LP-128-a |
| LP-404-a | LP-151-a |
| LP-404-a | LP-183-a |
| LP-404-a | LP-200-a |
| LP-404-a | LP-208-a |
| LP-404-a | LP-211-a |
| LP-404-a | LP-232-a |
| LP-404-a | LP-242-a |
| LP-404-a | LP-243-a |
| LP-404-a | LP-244-a |
| LP-404-a | LP-245-a |
| LP-404-a | LP-249-a |
| LP-404-a | LP-274-a |
| LP-404-a | LP-295-a |
| LP-404-a | LP-310-a |
| LP-404-a | LP-359-a |
| LP-404-a | LP-361-a |
| LP-404-a | LP-371-a |
| LP-404-a | LP-374-a |
| LP-404-a | LP-375-a |
| LP-404-a | LP-377-a |
| LP-404-a | LP-378-a |
| LP-404-a | LP-379-a |
| LP-404-a | LP-380-a |
| LP-404-a | LP-403-a |
| LP-404-a | LP-404-a |
| LP-404-a | LP-412-a |
| LP-404-a | LP-413-a |
| LP-404-a | LP-424-a |
| LP-404-a | LP-425-a |
| LP-404-a | LP-426-a |
| LP-404-a | LP-427-a |
| LP-404-a | LP-428-a |
| LP-404-a | LP-432-a |
| LP-404-a | LP-433-a |
| LP-404-a | LP-444-a |
| LP-404-a | LP-445-a |
| LP-404-a | LP-446-a |
| LP-404-a | LP-447-a |
| LP-404-a | LP-453-a |
| LP-404-a | LP-455-a |
| LP-404-a | LP-457-a |
| LP-404-a | LP-458-a |
| LP-404-a | LP-459-a |
| LP-404-a | LP-460-a |
| LP-404-a | LP-461-a |
| LP-404-a | LP-468-a |
| LP-404-a | LP-469-a |
| LP-404-a | LP-470-a |
| LP-404-a | LP-473-a |
| LP-404-a | LP-474-a |
| LP-404-a | CNR1 SM2-a |
| LP-404-a | (None) |
| LP-412-a | LP-4-a |
| LP-412-a | LP-18-a |
| LP-412-a | LP-128-a |
| LP-412-a | LP-151-a |
| LP-412-a | LP-183-a |
| LP-412-a | LP-200-a |
| LP-412-a | LP-208-a |
| LP-412-a | LP-211-a |
| LP-412-a | LP-232-a |
| LP-412-a | LP-242-a |
| LP-412-a | LP-243-a |
| LP-412-a | LP-244-a |
| LP-412-a | LP-245-a |
| LP-412-a | LP-249-a |
| LP-412-a | LP-274-a |
| LP-412-a | LP-295-a |
| LP-412-a | LP-310-a |
| LP-412-a | LP-359-a |
| LP-412-a | LP-361-a |
| LP-412-a | LP-371-a |
| LP-412-a | LP-374-a |
| LP-412-a | LP-375-a |
| LP-412-a | LP-377-a |
| LP-412-a | LP-378-a |
| LP-412-a | LP-379-a |
| LP-412-a | LP-380-a |
| LP-412-a | LP-403-a |
| LP-412-a | LP-404-a |
| LP-412-a | LP-412-a |
| LP-412-a | LP-413-a |
| LP-412-a | LP-424-a |
| LP-412-a | LP-425-a |
| LP-412-a | LP-426-a |
| LP-412-a | LP-427-a |
| LP-412-a | LP-428-a |
| LP-412-a | LP-432-a |
| LP-412-a | LP-433-a |
| LP-412-a | LP-444-a |
| LP-412-a | LP-445-a |
| LP-412-a | LP-446-a |
| LP-412-a | LP-447-a |
| LP-412-a | LP-453-a |
| LP-412-a | LP-455-a |
| LP-412-a | LP-457-a |
| LP-412-a | LP-458-a |
| LP-412-a | LP-459-a |
| LP-412-a | LP-460-a |
| LP-412-a | LP-461-a |
| LP-412-a | LP-468-a |
| LP-412-a | LP-469-a |
| LP-412-a | LP-470-a |
| LP-412-a | LP-473-a |
| LP-412-a | LP-474-a |
| LP-412-a | CNR1 SM2-a |
| LP-412-a | (None) |
| LP-413-a | LP-4-a |
| LP-413-a | LP-18-a |
| LP-413-a | LP-128-a |
| LP-413-a | LP-151-a |
| LP-413-a | LP-183-a |
| LP-413-a | LP-200-a |
| LP-413-a | LP-208-a |
| LP-413-a | LP-211-a |
| LP-413-a | LP-232-a |
| LP-413-a | LP-242-a |
| LP-413-a | LP-243-a |
| LP-413-a | LP-244-a |
| LP-413-a | LP-245-a |
| LP-413-a | LP-249-a |
| LP-413-a | LP-274-a |
| LP-413-a | LP-295-a |
| LP-413-a | LP-310-a |
| LP-413-a | LP-359-a |
| LP-413-a | LP-361-a |
| LP-413-a | LP-371-a |
| LP-413-a | LP-374-a |
| LP-413-a | LP-375-a |
| LP-413-a | LP-377-a |
| LP-413-a | LP-378-a |
| LP-413-a | LP-379-a |
| LP-413-a | LP-380-a |
| LP-413-a | LP-403-a |
| LP-413-a | LP-404-a |
| LP-413-a | LP-412-a |
| LP-413-a | LP-413-a |
| LP-413-a | LP-424-a |
| LP-413-a | LP-425-a |
| LP-413-a | LP-426-a |
| LP-413-a | LP-427-a |
| LP-413-a | LP-428-a |
| LP-413-a | LP-432-a |
| LP-413-a | LP-433-a |
| LP-413-a | LP-444-a |
| LP-413-a | LP-445-a |
| LP-413-a | LP-446-a |
| LP-413-a | LP-447-a |
| LP-413-a | LP-453-a |
| LP-413-a | LP-455-a |
| LP-413-a | LP-457-a |
| LP-413-a | LP-458-a |
| LP-413-a | LP-459-a |
| LP-413-a | LP-460-a |
| LP-413-a | LP-461-a |
| LP-413-a | LP-468-a |
| LP-413-a | LP-469-a |
| LP-413-a | LP-470-a |
| LP-413-a | LP-473-a |
| LP-413-a | LP-474-a |
| LP-413-a | CNR1 SM2-a |
| LP-413-a | (None) |
| LP-424-a | LP-4-a |
| LP-424-a | LP-18-a |
| LP-424-a | LP-128-a |
| LP-424-a | LP-151-a |
| LP-424-a | LP-183-a |
| LP-424-a | LP-200-a |
| LP-424-a | LP-208-a |
| LP-424-a | LP-211-a |
| LP-424-a | LP-232-a |
| LP-424-a | LP-242-a |
| LP-424-a | LP-243-a |
| LP-424-a | LP-244-a |
| LP-424-a | LP-245-a |
| LP-424-a | LP-249-a |
| LP-424-a | LP-274-a |
| LP-424-a | LP-295-a |
| LP-424-a | LP-310-a |
| LP-424-a | LP-359-a |
| LP-424-a | LP-361-a |
| LP-424-a | LP-371-a |
| LP-424-a | LP-374-a |
| LP-424-a | LP-375-a |
| LP-424-a | LP-377-a |
| LP-424-a | LP-378-a |
| LP-424-a | LP-379-a |
| LP-424-a | LP-380-a |
| LP-424-a | LP-403-a |
| LP-424-a | LP-404-a |
| LP-424-a | LP-412-a |
| LP-424-a | LP-413-a |
| LP-424-a | LP-424-a |
| LP-424-a | LP-425-a |
| LP-424-a | LP-426-a |
| LP-424-a | LP-427-a |
| LP-424-a | LP-428-a |
| LP-424-a | LP-432-a |
| LP-424-a | LP-433-a |
| LP-424-a | LP-444-a |
| LP-424-a | LP-445-a |
| LP-424-a | LP-446-a |
| LP-424-a | LP-447-a |
| LP-424-a | LP-453-a |
| LP-424-a | LP-455-a |
| LP-424-a | LP-457-a |
| LP-424-a | LP-458-a |
| LP-424-a | LP-459-a |
| LP-424-a | LP-460-a |
| LP-424-a | LP-461-a |
| LP-424-a | LP-468-a |
| LP-424-a | LP-469-a |
| LP-424-a | LP-470-a |
| LP-424-a | LP-473-a |
| LP-424-a | LP-474-a |
| LP-424-a | CNR1 SM2-a |
| LP-424-a | (None) |
| LP-425-a | LP-4-a |
| LP-425-a | LP-18-a |
| LP-425-a | LP-128-a |
| LP-425-a | LP-151-a |
| LP-425-a | LP-183-a |
| LP-425-a | LP-200-a |
| LP-425-a | LP-208-a |
| LP-425-a | LP-211-a |
| LP-425-a | LP-232-a |
| LP-425-a | LP-242-a |
| LP-425-a | LP-243-a |
| LP-425-a | LP-244-a |
| LP-425-a | LP-245-a |
| LP-425-a | LP-249-a |
| LP-425-a | LP-274-a |
| LP-425-a | LP-295-a |
| LP-425-a | LP-310-a |
| LP-425-a | LP-359-a |
| LP-425-a | LP-361-a |
| LP-425-a | LP-371-a |
| LP-425-a | LP-374-a |
| LP-425-a | LP-375-a |
| LP-425-a | LP-377-a |
| LP-425-a | LP-378-a |
| LP-425-a | LP-379-a |
| LP-425-a | LP-380-a |
| LP-425-a | LP-403-a |
| LP-425-a | LP-404-a |
| LP-425-a | LP-412-a |
| LP-425-a | LP-413-a |
| LP-425-a | LP-424-a |
| LP-425-a | LP-425-a |
| LP-425-a | LP-426-a |
| LP-425-a | LP-427-a |
| LP-425-a | LP-428-a |
| LP-425-a | LP-432-a |
| LP-425-a | LP-433-a |
| LP-425-a | LP-444-a |
| LP-425-a | LP-445-a |
| LP-425-a | LP-446-a |
| LP-425-a | LP-447-a |
| LP-425-a | LP-453-a |
| LP-425-a | LP-455-a |
| LP-425-a | LP-457-a |
| LP-425-a | LP-458-a |
| LP-425-a | LP-459-a |
| LP-425-a | LP-460-a |
| LP-425-a | LP-461-a |
| LP-425-a | LP-468-a |
| LP-425-a | LP-469-a |
| LP-425-a | LP-470-a |
| LP-425-a | LP-473-a |
| LP-425-a | LP-474-a |
| LP-425-a | CNR1 SM2-a |
| LP-425-a | (None) |
| LP-426-a | LP-4-a |
| LP-426-a | LP-18-a |
| LP-426-a | LP-128-a |
| LP-426-a | LP-151-a |
| LP-426-a | LP-183-a |
| LP-426-a | LP-200-a |
| LP-426-a | LP-208-a |
| LP-426-a | LP-211-a |
| LP-426-a | LP-232-a |
| LP-426-a | LP-242-a |
| LP-426-a | LP-243-a |
| LP-426-a | LP-244-a |
| LP-426-a | LP-245-a |
| LP-426-a | LP-249-a |
| LP-426-a | LP-274-a |
| LP-426-a | LP-295-a |
| LP-426-a | LP-310-a |
| LP-426-a | LP-359-a |
| LP-426-a | LP-361-a |
| LP-426-a | LP-371-a |
| LP-426-a | LP-374-a |
| LP-426-a | LP-375-a |
| LP-426-a | LP-377-a |
| LP-426-a | LP-378-a |
| LP-426-a | LP-379-a |
| LP-426-a | LP-380-a |
| LP-426-a | LP-403-a |
| LP-426-a | LP-404-a |
| LP-426-a | LP-412-a |
| LP-426-a | LP-413-a |
| LP-426-a | LP-424-a |
| LP-426-a | LP-425-a |
| LP-426-a | LP-426-a |
| LP-426-a | LP-427-a |
| LP-426-a | LP-428-a |
| LP-426-a | LP-432-a |
| LP-426-a | LP-433-a |
| LP-426-a | LP-444-a |
| LP-426-a | LP-445-a |
| LP-426-a | LP-446-a |
| LP-426-a | LP-447-a |
| LP-426-a | LP-453-a |
| LP-426-a | LP-455-a |
| LP-426-a | LP-457-a |
| LP-426-a | LP-458-a |
| LP-426-a | LP-459-a |
| LP-426-a | LP-460-a |
| LP-426-a | LP-461-a |
| LP-426-a | LP-468-a |
| LP-426-a | LP-469-a |
| LP-426-a | LP-470-a |
| LP-426-a | LP-473-a |
| LP-426-a | LP-474-a |
| LP-426-a | CNR1 SM2-a |
| LP-426-a | (None) |
| LP-427-a | LP-4-a |
| LP-427-a | LP-18-a |
| LP-427-a | LP-128-a |
| LP-427-a | LP-151-a |
| LP-427-a | LP-183-a |
| LP-427-a | LP-200-a |
| LP-427-a | LP-208-a |
| LP-427-a | LP-211-a |
| LP-427-a | LP-232-a |
| LP-427-a | LP-242-a |
| LP-427-a | LP-243-a |
| LP-427-a | LP-244-a |
| LP-427-a | LP-245-a |
| LP-427-a | LP-249-a |
| LP-427-a | LP-274-a |
| LP-427-a | LP-295-a |
| LP-427-a | LP-310-a |
| LP-427-a | LP-359-a |
| LP-427-a | LP-361-a |
| LP-427-a | LP-371-a |
| LP-427-a | LP-374-a |
| LP-427-a | LP-375-a |
| LP-427-a | LP-377-a |
| LP-427-a | LP-378-a |
| LP-427-a | LP-379-a |
| LP-427-a | LP-380-a |
| LP-427-a | LP-403-a |
| LP-427-a | LP-404-a |
| LP-427-a | LP-412-a |
| LP-427-a | LP-413-a |
| LP-427-a | LP-424-a |
| LP-427-a | LP-425-a |
| LP-427-a | LP-426-a |
| LP-427-a | LP-427-a |
| LP-427-a | LP-428-a |
| LP-427-a | LP-432-a |
| LP-427-a | LP-433-a |
| LP-427-a | LP-444-a |
| LP-427-a | LP-445-a |
| LP-427-a | LP-446-a |
| LP-427-a | LP-447-a |
| LP-427-a | LP-453-a |
| LP-427-a | LP-455-a |
| LP-427-a | LP-457-a |
| LP-427-a | LP-458-a |
| LP-427-a | LP-459-a |
| LP-427-a | LP-460-a |
| LP-427-a | LP-461-a |
| LP-427-a | LP-468-a |
| LP-427-a | LP-469-a |
| LP-427-a | LP-470-a |
| LP-427-a | LP-473-a |
| LP-427-a | LP-474-a |
| LP-427-a | CNR1 SM2-a |
| LP-427-a | (None) |
| LP-428-a | LP-4-a |
| LP-428-a | LP-18-a |
| LP-428-a | LP-128-a |
| LP-428-a | LP-151-a |
| LP-428-a | LP-183-a |
| LP-428-a | LP-200-a |
| LP-428-a | LP-208-a |
| LP-428-a | LP-211-a |
| LP-428-a | LP-232-a |
| LP-428-a | LP-242-a |
| LP-428-a | LP-243-a |
| LP-428-a | LP-244-a |
| LP-428-a | LP-245-a |
| LP-428-a | LP-249-a |
| LP-428-a | LP-274-a |
| LP-428-a | LP-295-a |
| LP-428-a | LP-310-a |
| LP-428-a | LP-359-a |
| LP-428-a | LP-361-a |
| LP-428-a | LP-371-a |
| LP-428-a | LP-374-a |
| LP-428-a | LP-375-a |
| LP-428-a | LP-377-a |
| LP-428-a | LP-378-a |
| LP-428-a | LP-379-a |
| LP-428-a | LP-380-a |
| LP-428-a | LP-403-a |
| LP-428-a | LP-404-a |
| LP-428-a | LP-412-a |
| LP-428-a | LP-413-a |
| LP-428-a | LP-424-a |
| LP-428-a | LP-425-a |
| LP-428-a | LP-426-a |
| LP-428-a | LP-427-a |
| LP-428-a | LP-428-a |
| LP-428-a | LP-432-a |
| LP-428-a | LP-433-a |
| LP-428-a | LP-444-a |
| LP-428-a | LP-445-a |
| LP-428-a | LP-446-a |
| LP-428-a | LP-447-a |
| LP-428-a | LP-453-a |
| LP-428-a | LP-455-a |
| LP-428-a | LP-457-a |
| LP-428-a | LP-458-a |
| LP-428-a | LP-459-a |
| LP-428-a | LP-460-a |
| LP-428-a | LP-461-a |
| LP-428-a | LP-468-a |
| LP-428-a | LP-469-a |
| LP-428-a | LP-470-a |
| LP-428-a | LP-473-a |
| LP-428-a | LP-474-a |
| LP-428-a | CNR1 SM2-a |
| LP-428-a | (None) |
| LP-432-a | LP-4-a |
| LP-432-a | LP-18-a |
| LP-432-a | LP-128-a |
| LP-432-a | LP-151-a |
| LP-432-a | LP-183-a |
| LP-432-a | LP-200-a |
| LP-432-a | LP-208-a |
| LP-432-a | LP-211-a |
| LP-432-a | LP-232-a |
| LP-432-a | LP-242-a |
| LP-432-a | LP-243-a |
| LP-432-a | LP-244-a |
| LP-432-a | LP-245-a |
| LP-432-a | LP-249-a |
| LP-432-a | LP-274-a |
| LP-432-a | LP-295-a |
| LP-432-a | LP-310-a |
| LP-432-a | LP-359-a |
| LP-432-a | LP-361-a |
| LP-432-a | LP-371-a |
| LP-432-a | LP-374-a |
| LP-432-a | LP-375-a |
| LP-432-a | LP-377-a |
| LP-432-a | LP-378-a |
| LP-432-a | LP-379-a |
| LP-432-a | LP-380-a |
| LP-432-a | LP-403-a |
| LP-432-a | LP-404-a |
| LP-432-a | LP-412-a |
| LP-432-a | LP-413-a |
| LP-432-a | LP-424-a |
| LP-432-a | LP-425-a |
| LP-432-a | LP-426-a |
| LP-432-a | LP-427-a |
| LP-432-a | LP-428-a |
| LP-432-a | LP-432-a |
| LP-432-a | LP-433-a |
| LP-432-a | LP-444-a |
| LP-432-a | LP-445-a |
| LP-432-a | LP-446-a |
| LP-432-a | LP-447-a |
| LP-432-a | LP-453-a |
| LP-432-a | LP-455-a |
| LP-432-a | LP-457-a |
| LP-432-a | LP-458-a |
| LP-432-a | LP-459-a |
| LP-432-a | LP-460-a |
| LP-432-a | LP-461-a |
| LP-432-a | LP-468-a |
| LP-432-a | LP-469-a |
| LP-432-a | LP-470-a |
| LP-432-a | LP-473-a |
| LP-432-a | LP-474-a |
| LP-432-a | CNR1 SM2-a |
| LP-432-a | (None) |
| LP-433-a | LP-4-a |
| LP-433-a | LP-18-a |
| LP-433-a | LP-128-a |
| LP-433-a | LP-151-a |
| LP-433-a | LP-183-a |
| LP-433-a | LP-200-a |
| LP-433-a | LP-208-a |
| LP-433-a | LP-211-a |
| LP-433-a | LP-232-a |
| LP-433-a | LP-242-a |
| LP-433-a | LP-243-a |
| LP-433-a | LP-244-a |
| LP-433-a | LP-245-a |
| LP-433-a | LP-249-a |
| LP-433-a | LP-274-a |
| LP-433-a | LP-295-a |
| LP-433-a | LP-310-a |
| LP-433-a | LP-359-a |
| LP-433-a | LP-361-a |
| LP-433-a | LP-371-a |
| LP-433-a | LP-374-a |
| LP-433-a | LP-375-a |
| LP-433-a | LP-377-a |
| LP-433-a | LP-378-a |
| LP-433-a | LP-379-a |
| LP-433-a | LP-380-a |
| LP-433-a | LP-403-a |
| LP-433-a | LP-404-a |
| LP-433-a | LP-412-a |
| LP-433-a | LP-413-a |
| LP-433-a | LP-424-a |
| LP-433-a | LP-425-a |
| LP-433-a | LP-426-a |
| LP-433-a | LP-427-a |
| LP-433-a | LP-428-a |
| LP-433-a | LP-432-a |
| LP-433-a | LP-433-a |
| LP-433-a | LP-444-a |
| LP-433-a | LP-445-a |
| LP-433-a | LP-446-a |
| LP-433-a | LP-447-a |
| LP-433-a | LP-453-a |
| LP-433-a | LP-455-a |
| LP-433-a | LP-457-a |
| LP-433-a | LP-458-a |
| LP-433-a | LP-459-a |
| LP-433-a | LP-460-a |
| LP-433-a | LP-461-a |
| LP-433-a | LP-468-a |
| LP-433-a | LP-469-a |
| LP-433-a | LP-470-a |
| LP-433-a | LP-473-a |
| LP-433-a | LP-474-a |
| LP-433-a | CNR1 SM2-a |
| LP-433-a | (None) |
| LP-444-a | LP-4-a |
| LP-444-a | LP-18-a |
| LP-444-a | LP-128-a |
| LP-444-a | LP-151-a |
| LP-444-a | LP-183-a |
| LP-444-a | LP-200-a |
| LP-444-a | LP-208-a |
| LP-444-a | LP-211-a |
| LP-444-a | LP-232-a |
| LP-444-a | LP-242-a |
| LP-444-a | LP-243-a |
| LP-444-a | LP-244-a |
| LP-444-a | LP-245-a |
| LP-444-a | LP-249-a |
| LP-444-a | LP-274-a |
| LP-444-a | LP-295-a |
| LP-444-a | LP-310-a |
| LP-444-a | LP-359-a |
| LP-444-a | LP-361-a |
| LP-444-a | LP-371-a |
| LP-444-a | LP-374-a |
| LP-444-a | LP-375-a |
| LP-444-a | LP-377-a |
| LP-444-a | LP-378-a |
| LP-444-a | LP-379-a |
| LP-444-a | LP-380-a |
| LP-444-a | LP-403-a |
| LP-444-a | LP-404-a |
| LP-444-a | LP-412-a |
| LP-444-a | LP-413-a |
| LP-444-a | LP-424-a |
| LP-444-a | LP-425-a |
| LP-444-a | LP-426-a |
| LP-444-a | LP-427-a |
| LP-444-a | LP-428-a |
| LP-444-a | LP-432-a |
| LP-444-a | LP-433-a |
| LP-444-a | LP-444-a |
| LP-444-a | LP-445-a |
| LP-444-a | LP-446-a |
| LP-444-a | LP-447-a |
| LP-444-a | LP-453-a |
| LP-444-a | LP-455-a |
| LP-444-a | LP-457-a |
| LP-444-a | LP-458-a |
| LP-444-a | LP-459-a |
| LP-444-a | LP-460-a |
| LP-444-a | LP-461-a |
| LP-444-a | LP-468-a |
| LP-444-a | LP-469-a |
| LP-444-a | LP-470-a |
| LP-444-a | LP-473-a |
| LP-444-a | LP-474-a |
| LP-444-a | CNR1 SM2-a |
| LP-444-a | (None) |
| LP-445-a | LP-4-a |
| LP-445-a | LP-18-a |
| LP-445-a | LP-128-a |
| LP-445-a | LP-151-a |
| LP-445-a | LP-183-a |
| LP-445-a | LP-200-a |
| LP-445-a | LP-208-a |
| LP-445-a | LP-211-a |
| LP-445-a | LP-232-a |
| LP-445-a | LP-242-a |
| LP-445-a | LP-243-a |
| LP-445-a | LP-244-a |
| LP-445-a | LP-245-a |
| LP-445-a | LP-249-a |
| LP-445-a | LP-274-a |
| LP-445-a | LP-295-a |
| LP-445-a | LP-310-a |
| LP-445-a | LP-359-a |
| LP-445-a | LP-361-a |
| LP-445-a | LP-371-a |
| LP-445-a | LP-374-a |
| LP-445-a | LP-375-a |
| LP-445-a | LP-377-a |
| LP-445-a | LP-378-a |
| LP-445-a | LP-379-a |
| LP-445-a | LP-380-a |
| LP-445-a | LP-403-a |
| LP-445-a | LP-404-a |
| LP-445-a | LP-412-a |
| LP-445-a | LP-413-a |
| LP-445-a | LP-424-a |
| LP-445-a | LP-425-a |
| LP-445-a | LP-426-a |
| LP-445-a | LP-427-a |
| LP-445-a | LP-428-a |
| LP-445-a | LP-432-a |
| LP-445-a | LP-433-a |
| LP-445-a | LP-444-a |
| LP-445-a | LP-445-a |
| LP-445-a | LP-446-a |
| LP-445-a | LP-447-a |
| LP-445-a | LP-453-a |
| LP-445-a | LP-455-a |
| LP-445-a | LP-457-a |
| LP-445-a | LP-458-a |
| LP-445-a | LP-459-a |
| LP-445-a | LP-460-a |
| LP-445-a | LP-461-a |
| LP-445-a | LP-468-a |
| LP-445-a | LP-469-a |
| LP-445-a | LP-470-a |
| LP-445-a | LP-473-a |
| LP-445-a | LP-474-a |
| LP-445-a | CNR1 SM2-a |
| LP-445-a | (None) |
| LP-446-a | LP-4-a |
| LP-446-a | LP-18-a |
| LP-446-a | LP-128-a |
| LP-446-a | LP-151-a |
| LP-446-a | LP-183-a |
| LP-446-a | LP-200-a |
| LP-446-a | LP-208-a |
| LP-446-a | LP-211-a |
| LP-446-a | LP-232-a |
| LP-446-a | LP-242-a |
| LP-446-a | LP-243-a |
| LP-446-a | LP-244-a |
| LP-446-a | LP-245-a |
| LP-446-a | LP-249-a |
| LP-446-a | LP-274-a |
| LP-446-a | LP-295-a |
| LP-446-a | LP-310-a |
| LP-446-a | LP-359-a |
| LP-446-a | LP-361-a |
| LP-446-a | LP-371-a |
| LP-446-a | LP-374-a |
| LP-446-a | LP-375-a |
| LP-446-a | LP-377-a |
| LP-446-a | LP-378-a |
| LP-446-a | LP-379-a |
| LP-446-a | LP-380-a |
| LP-446-a | LP-403-a |
| LP-446-a | LP-404-a |
| LP-446-a | LP-412-a |
| LP-446-a | LP-413-a |
| LP-446-a | LP-424-a |
| LP-446-a | LP-425-a |
| LP-446-a | LP-426-a |
| LP-446-a | LP-427-a |
| LP-446-a | LP-428-a |
| LP-446-a | LP-432-a |
| LP-446-a | LP-433-a |
| LP-446-a | LP-444-a |
| LP-446-a | LP-445-a |
| LP-446-a | LP-446-a |
| LP-446-a | LP-447-a |
| LP-446-a | LP-453-a |
| LP-446-a | LP-455-a |
| LP-446-a | LP-457-a |
| LP-446-a | LP-458-a |
| LP-446-a | LP-459-a |
| LP-446-a | LP-460-a |
| LP-446-a | LP-461-a |
| LP-446-a | LP-468-a |
| LP-446-a | LP-469-a |
| LP-446-a | LP-470-a |
| LP-446-a | LP-473-a |
| LP-446-a | LP-474-a |
| LP-446-a | CNR1 SM2-a |
| LP-446-a | (None) |
| LP-447-a | LP-4-a |
| LP-447-a | LP-18-a |
| LP-447-a | LP-128-a |
| LP-447-a | LP-151-a |
| LP-447-a | LP-183-a |
| LP-447-a | LP-200-a |
| LP-447-a | LP-208-a |
| LP-447-a | LP-211-a |
| LP-447-a | LP-232-a |
| LP-447-a | LP-242-a |
| LP-447-a | LP-243-a |
| LP-447-a | LP-244-a |
| LP-447-a | LP-245-a |
| LP-447-a | LP-249-a |
| LP-447-a | LP-274-a |
| LP-447-a | LP-295-a |
| LP-447-a | LP-310-a |
| LP-447-a | LP-359-a |
| LP-447-a | LP-361-a |
| LP-447-a | LP-371-a |
| LP-447-a | LP-374-a |
| LP-447-a | LP-375-a |
| LP-447-a | LP-377-a |
| LP-447-a | LP-378-a |
| LP-447-a | LP-379-a |
| LP-447-a | LP-380-a |
| LP-447-a | LP-403-a |
| LP-447-a | LP-404-a |
| LP-447-a | LP-412-a |
| LP-447-a | LP-413-a |
| LP-447-a | LP-424-a |
| LP-447-a | LP-425-a |
| LP-447-a | LP-426-a |
| LP-447-a | LP-427-a |
| LP-447-a | LP-428-a |
| LP-447-a | LP-432-a |
| LP-447-a | LP-433-a |
| LP-447-a | LP-444-a |
| LP-447-a | LP-445-a |
| LP-447-a | LP-446-a |
| LP-447-a | LP-447-a |
| LP-447-a | LP-453-a |
| LP-447-a | LP-455-a |
| LP-447-a | LP-457-a |
| LP-447-a | LP-458-a |
| LP-447-a | LP-459-a |
| LP-447-a | LP-460-a |
| LP-447-a | LP-461-a |
| LP-447-a | LP-468-a |
| LP-447-a | LP-469-a |
| LP-447-a | LP-470-a |
| LP-447-a | LP-473-a |
| LP-447-a | LP-474-a |
| LP-447-a | CNR1 SM2-a |
| LP-447-a | (None) |
| LP-453-a | LP-4-a |
| LP-453-a | LP-18-a |
| LP-453-a | LP-128-a |
| LP-453-a | LP-151-a |
| LP-453-a | LP-183-a |
| LP-453-a | LP-200-a |
| LP-453-a | LP-208-a |
| LP-453-a | LP-211-a |
| LP-453-a | LP-232-a |
| LP-453-a | LP-242-a |
| LP-453-a | LP-243-a |
| LP-453-a | LP-244-a |
| LP-453-a | LP-245-a |
| LP-453-a | LP-249-a |
| LP-453-a | LP-274-a |
| LP-453-a | LP-295-a |
| LP-453-a | LP-310-a |
| LP-453-a | LP-359-a |
| LP-453-a | LP-361-a |
| LP-453-a | LP-371-a |
| LP-453-a | LP-374-a |
| LP-453-a | LP-375-a |
| LP-453-a | LP-377-a |
| LP-453-a | LP-378-a |
| LP-453-a | LP-379-a |
| LP-453-a | LP-380-a |
| LP-453-a | LP-403-a |
| LP-453-a | LP-404-a |
| LP-453-a | LP-412-a |
| LP-453-a | LP-413-a |
| LP-453-a | LP-424-a |
| LP-453-a | LP-425-a |
| LP-453-a | LP-426-a |
| LP-453-a | LP-427-a |
| LP-453-a | LP-428-a |
| LP-453-a | LP-432-a |
| LP-453-a | LP-433-a |
| LP-453-a | LP-444-a |
| LP-453-a | LP-445-a |
| LP-453-a | LP-446-a |
| LP-453-a | LP-447-a |
| LP-453-a | LP-453-a |
| LP-453-a | LP-455-a |
| LP-453-a | LP-457-a |
| LP-453-a | LP-458-a |
| LP-453-a | LP-459-a |
| LP-453-a | LP-460-a |
| LP-453-a | LP-461-a |
| LP-453-a | LP-468-a |
| LP-453-a | LP-469-a |
| LP-453-a | LP-470-a |
| LP-453-a | LP-473-a |
| LP-453-a | LP-474-a |
| LP-453-a | CNR1 SM2-a |
| LP-453-a | (None) |
| LP-455-a | LP-4-a |
| LP-455-a | LP-18-a |
| LP-455-a | LP-128-a |
| LP-455-a | LP-151-a |
| LP-455-a | LP-183-a |
| LP-455-a | LP-200-a |
| LP-455-a | LP-208-a |
| LP-455-a | LP-211-a |
| LP-455-a | LP-232-a |
| LP-455-a | LP-242-a |
| LP-455-a | LP-243-a |
| LP-455-a | LP-244-a |
| LP-455-a | LP-245-a |
| LP-455-a | LP-249-a |
| LP-455-a | LP-274-a |
| LP-455-a | LP-295-a |
| LP-455-a | LP-310-a |
| LP-455-a | LP-359-a |
| LP-455-a | LP-361-a |
| LP-455-a | LP-371-a |
| LP-455-a | LP-374-a |
| LP-455-a | LP-375-a |
| LP-455-a | LP-377-a |
| LP-455-a | LP-378-a |
| LP-455-a | LP-379-a |
| LP-455-a | LP-380-a |
| LP-455-a | LP-403-a |
| LP-455-a | LP-404-a |
| LP-455-a | LP-412-a |
| LP-455-a | LP-413-a |
| LP-455-a | LP-424-a |
| LP-455-a | LP-425-a |
| LP-455-a | LP-426-a |
| LP-455-a | LP-427-a |
| LP-455-a | LP-428-a |
| LP-455-a | LP-432-a |
| LP-455-a | LP-433-a |
| LP-455-a | LP-444-a |
| LP-455-a | LP-445-a |
| LP-455-a | LP-446-a |
| LP-455-a | LP-447-a |
| LP-455-a | LP-453-a |
| LP-455-a | LP-455-a |
| LP-455-a | LP-457-a |
| LP-455-a | LP-458-a |
| LP-455-a | LP-459-a |
| LP-455-a | LP-460-a |
| LP-455-a | LP-461-a |
| LP-455-a | LP-468-a |
| LP-455-a | LP-469-a |
| LP-455-a | LP-470-a |
| LP-455-a | LP-473-a |
| LP-455-a | LP-474-a |
| LP-455-a | CNR1 SM2-a |
| LP-455-a | (None) |
| LP-457-a | LP-4-a |
| LP-457-a | LP-18-a |
| LP-457-a | LP-128-a |
| LP-457-a | LP-151-a |
| LP-457-a | LP-183-a |
| LP-457-a | LP-200-a |
| LP-457-a | LP-208-a |
| LP-457-a | LP-211-a |
| LP-457-a | LP-232-a |
| LP-457-a | LP-242-a |
| LP-457-a | LP-243-a |
| LP-457-a | LP-244-a |
| LP-457-a | LP-245-a |
| LP-457-a | LP-249-a |
| LP-457-a | LP-274-a |
| LP-457-a | LP-295-a |
| LP-457-a | LP-310-a |
| LP-457-a | LP-359-a |
| LP-457-a | LP-361-a |
| LP-457-a | LP-371-a |
| LP-457-a | LP-374-a |
| LP-457-a | LP-375-a |
| LP-457-a | LP-377-a |
| LP-457-a | LP-378-a |
| LP-457-a | LP-379-a |
| LP-457-a | LP-380-a |
| LP-457-a | LP-403-a |
| LP-457-a | LP-404-a |
| LP-457-a | LP-412-a |
| LP-457-a | LP-413-a |
| LP-457-a | LP-424-a |
| LP-457-a | LP-425-a |
| LP-457-a | LP-426-a |
| LP-457-a | LP-427-a |
| LP-457-a | LP-428-a |
| LP-457-a | LP-432-a |
| LP-457-a | LP-433-a |
| LP-457-a | LP-444-a |
| LP-457-a | LP-445-a |
| LP-457-a | LP-446-a |
| LP-457-a | LP-447-a |
| LP-457-a | LP-453-a |
| LP-457-a | LP-455-a |
| LP-457-a | LP-457-a |
| LP-457-a | LP-458-a |
| LP-457-a | LP-459-a |
| LP-457-a | LP-460-a |
| LP-457-a | LP-461-a |
| LP-457-a | LP-468-a |
| LP-457-a | LP-469-a |
| LP-457-a | LP-470-a |
| LP-457-a | LP-473-a |
| LP-457-a | LP-474-a |
| LP-457-a | CNR1 SM2-a |
| LP-457-a | (None) |
| LP-458-a | LP-4-a |
| LP-458-a | LP-18-a |
| LP-458-a | LP-128-a |
| LP-458-a | LP-151-a |
| LP-458-a | LP-183-a |
| LP-458-a | LP-200-a |
| LP-458-a | LP-208-a |
| LP-458-a | LP-211-a |
| LP-458-a | LP-232-a |
| LP-458-a | LP-242-a |
| LP-458-a | LP-243-a |
| LP-458-a | LP-244-a |
| LP-458-a | LP-245-a |
| LP-458-a | LP-249-a |
| LP-458-a | LP-274-a |
| LP-458-a | LP-295-a |
| LP-458-a | LP-310-a |
| LP-458-a | LP-359-a |
| LP-458-a | LP-361-a |
| LP-458-a | LP-371-a |
| LP-458-a | LP-374-a |
| LP-458-a | LP-375-a |
| LP-458-a | LP-377-a |
| LP-458-a | LP-378-a |
| LP-458-a | LP-379-a |
| LP-458-a | LP-380-a |
| LP-458-a | LP-403-a |
| LP-458-a | LP-404-a |
| LP-458-a | LP-412-a |
| LP-458-a | LP-413-a |
| LP-458-a | LP-424-a |
| LP-458-a | LP-425-a |
| LP-458-a | LP-426-a |
| LP-458-a | LP-427-a |
| LP-458-a | LP-428-a |
| LP-458-a | LP-432-a |
| LP-458-a | LP-433-a |
| LP-458-a | LP-444-a |
| LP-458-a | LP-445-a |
| LP-458-a | LP-446-a |
| LP-458-a | LP-447-a |
| LP-458-a | LP-453-a |
| LP-458-a | LP-455-a |
| LP-458-a | LP-457-a |
| LP-458-a | LP-458-a |
| LP-458-a | LP-459-a |
| LP-458-a | LP-460-a |
| LP-458-a | LP-461-a |
| LP-458-a | LP-468-a |
| LP-458-a | LP-469-a |
| LP-458-a | LP-470-a |
| LP-458-a | LP-473-a |
| LP-458-a | LP-474-a |
| LP-458-a | CNR1 SM2-a |
| LP-458-a | (None) |
| LP-459-a | LP-4-a |
| LP-459-a | LP-18-a |
| LP-459-a | LP-128-a |
| LP-459-a | LP-151-a |
| LP-459-a | LP-183-a |
| LP-459-a | LP-200-a |
| LP-459-a | LP-208-a |
| LP-459-a | LP-211-a |
| LP-459-a | LP-232-a |
| LP-459-a | LP-242-a |
| LP-459-a | LP-243-a |
| LP-459-a | LP-244-a |
| LP-459-a | LP-245-a |
| LP-459-a | LP-249-a |
| LP-459-a | LP-274-a |
| LP-459-a | LP-295-a |
| LP-459-a | LP-310-a |
| LP-459-a | LP-359-a |
| LP-459-a | LP-361-a |
| LP-459-a | LP-371-a |
| LP-459-a | LP-374-a |
| LP-459-a | LP-375-a |
| LP-459-a | LP-377-a |
| LP-459-a | LP-378-a |
| LP-459-a | LP-379-a |
| LP-459-a | LP-380-a |
| LP-459-a | LP-403-a |
| LP-459-a | LP-404-a |
| LP-459-a | LP-412-a |
| LP-459-a | LP-413-a |
| LP-459-a | LP-424-a |
| LP-459-a | LP-425-a |
| LP-459-a | LP-426-a |
| LP-459-a | LP-427-a |
| LP-459-a | LP-428-a |
| LP-459-a | LP-432-a |
| LP-459-a | LP-433-a |
| LP-459-a | LP-444-a |
| LP-459-a | LP-445-a |
| LP-459-a | LP-446-a |
| LP-459-a | LP-447-a |
| LP-459-a | LP-453-a |
| LP-459-a | LP-455-a |
| LP-459-a | LP-457-a |
| LP-459-a | LP-458-a |
| LP-459-a | LP-459-a |
| LP-459-a | LP-460-a |
| LP-459-a | LP-461-a |
| LP-459-a | LP-468-a |
| LP-459-a | LP-469-a |
| LP-459-a | LP-470-a |
| LP-459-a | LP-473-a |
| LP-459-a | LP-474-a |
| LP-459-a | CNR1 SM2-a |
| LP-459-a | (None) |
| LP-460-a | LP-4-a |
| LP-460-a | LP-18-a |
| LP-460-a | LP-128-a |
| LP-460-a | LP-151-a |
| LP-460-a | LP-183-a |
| LP-460-a | LP-200-a |
| LP-460-a | LP-208-a |
| LP-460-a | LP-211-a |
| LP-460-a | LP-232-a |
| LP-460-a | LP-242-a |
| LP-460-a | LP-243-a |
| LP-460-a | LP-244-a |
| LP-460-a | LP-245-a |
| LP-460-a | LP-249-a |
| LP-460-a | LP-274-a |
| LP-460-a | LP-295-a |
| LP-460-a | LP-310-a |
| LP-460-a | LP-359-a |
| LP-460-a | LP-361-a |
| LP-460-a | LP-371-a |
| LP-460-a | LP-374-a |
| LP-460-a | LP-375-a |
| LP-460-a | LP-377-a |
| LP-460-a | LP-378-a |
| LP-460-a | LP-379-a |
| LP-460-a | LP-380-a |
| LP-460-a | LP-403-a |
| LP-460-a | LP-404-a |
| LP-460-a | LP-412-a |
| LP-460-a | LP-413-a |
| LP-460-a | LP-424-a |
| LP-460-a | LP-425-a |
| LP-460-a | LP-426-a |
| LP-460-a | LP-427-a |
| LP-460-a | LP-428-a |
| LP-460-a | LP-432-a |
| LP-460-a | LP-433-a |
| LP-460-a | LP-444-a |
| LP-460-a | LP-445-a |
| LP-460-a | LP-446-a |
| LP-460-a | LP-447-a |
| LP-460-a | LP-453-a |
| LP-460-a | LP-455-a |
| LP-460-a | LP-457-a |
| LP-460-a | LP-458-a |
| LP-460-a | LP-459-a |
| LP-460-a | LP-460-a |
| LP-460-a | LP-461-a |
| LP-460-a | LP-468-a |
| LP-460-a | LP-469-a |
| LP-460-a | LP-470-a |
| LP-460-a | LP-473-a |
| LP-460-a | LP-474-a |
| LP-460-a | CNR1 SM2-a |
| LP-460-a | (None) |
| LP-461-a | LP-4-a |
| LP-461-a | LP-18-a |
| LP-461-a | LP-128-a |
| LP-461-a | LP-151-a |
| LP-461-a | LP-183-a |
| LP-461-a | LP-200-a |
| LP-461-a | LP-208-a |
| LP-461-a | LP-211-a |
| LP-461-a | LP-232-a |
| LP-461-a | LP-242-a |
| LP-461-a | LP-243-a |
| LP-461-a | LP-244-a |
| LP-461-a | LP-245-a |
| LP-461-a | LP-249-a |
| LP-461-a | LP-274-a |
| LP-461-a | LP-295-a |
| LP-461-a | LP-310-a |
| LP-461-a | LP-359-a |
| LP-461-a | LP-361-a |
| LP-461-a | LP-371-a |
| LP-461-a | LP-374-a |
| LP-461-a | LP-375-a |
| LP-461-a | LP-377-a |
| LP-461-a | LP-378-a |
| LP-461-a | LP-379-a |
| LP-461-a | LP-380-a |
| LP-461-a | LP-403-a |
| LP-461-a | LP-404-a |
| LP-461-a | LP-412-a |
| LP-461-a | LP-413-a |
| LP-461-a | LP-424-a |
| LP-461-a | LP-425-a |
| LP-461-a | LP-426-a |
| LP-461-a | LP-427-a |
| LP-461-a | LP-428-a |
| LP-461-a | LP-432-a |
| LP-461-a | LP-433-a |
| LP-461-a | LP-444-a |
| LP-461-a | LP-445-a |
| LP-461-a | LP-446-a |
| LP-461-a | LP-447-a |
| LP-461-a | LP-453-a |
| LP-461-a | LP-455-a |
| LP-461-a | LP-457-a |
| LP-461-a | LP-458-a |
| LP-461-a | LP-459-a |
| LP-461-a | LP-460-a |
| LP-461-a | LP-461-a |
| LP-461-a | LP-468-a |
| LP-461-a | LP-469-a |
| LP-461-a | LP-470-a |
| LP-461-a | LP-473-a |
| LP-461-a | LP-474-a |
| LP-461-a | CNR1 SM2-a |
| LP-461-a | (None) |
| LP-468-a | LP-4-a |
| LP-468-a | LP-18-a |
| LP-468-a | LP-128-a |
| LP-468-a | LP-151-a |
| LP-468-a | LP-183-a |
| LP-468-a | LP-200-a |
| LP-468-a | LP-208-a |
| LP-468-a | LP-211-a |
| LP-468-a | LP-232-a |
| LP-468-a | LP-242-a |
| LP-468-a | LP-243-a |
| LP-468-a | LP-244-a |
| LP-468-a | LP-245-a |
| LP-468-a | LP-249-a |
| LP-468-a | LP-274-a |
| LP-468-a | LP-295-a |
| LP-468-a | LP-310-a |
| LP-468-a | LP-359-a |
| LP-468-a | LP-361-a |
| LP-468-a | LP-371-a |
| LP-468-a | LP-374-a |
| LP-468-a | LP-375-a |
| LP-468-a | LP-377-a |
| LP-468-a | LP-378-a |
| LP-468-a | LP-379-a |
| LP-468-a | LP-380-a |
| LP-468-a | LP-403-a |
| LP-468-a | LP-404-a |
| LP-468-a | LP-412-a |
| LP-468-a | LP-413-a |
| LP-468-a | LP-424-a |
| LP-468-a | LP-425-a |
| LP-468-a | LP-426-a |
| LP-468-a | LP-427-a |
| LP-468-a | LP-428-a |
| LP-468-a | LP-432-a |
| LP-468-a | LP-433-a |
| LP-468-a | LP-444-a |
| LP-468-a | LP-445-a |
| LP-468-a | LP-446-a |
| LP-468-a | LP-447-a |
| LP-468-a | LP-453-a |
| LP-468-a | LP-455-a |
| LP-468-a | LP-457-a |
| LP-468-a | LP-458-a |
| LP-468-a | LP-459-a |
| LP-468-a | LP-460-a |
| LP-468-a | LP-461-a |
| LP-468-a | LP-468-a |
| LP-468-a | LP-469-a |
| LP-468-a | LP-470-a |
| LP-468-a | LP-473-a |
| LP-468-a | LP-474-a |
| LP-468-a | CNR1 SM2-a |
| LP-468-a | (None) |
| LP-469-a | LP-4-a |
| LP-469-a | LP-18-a |
| LP-469-a | LP-128-a |
| LP-469-a | LP-151-a |
| LP-469-a | LP-183-a |
| LP-469-a | LP-200-a |
| LP-469-a | LP-208-a |
| LP-469-a | LP-211-a |
| LP-469-a | LP-232-a |
| LP-469-a | LP-242-a |
| LP-469-a | LP-243-a |
| LP-469-a | LP-244-a |
| LP-469-a | LP-245-a |
| LP-469-a | LP-249-a |
| LP-469-a | LP-274-a |
| LP-469-a | LP-295-a |
| LP-469-a | LP-310-a |
| LP-469-a | LP-359-a |
| LP-469-a | LP-361-a |
| LP-469-a | LP-371-a |
| LP-469-a | LP-374-a |
| LP-469-a | LP-375-a |
| LP-469-a | LP-377-a |
| LP-469-a | LP-378-a |
| LP-469-a | LP-379-a |
| LP-469-a | LP-380-a |
| LP-469-a | LP-403-a |
| LP-469-a | LP-404-a |
| LP-469-a | LP-412-a |
| LP-469-a | LP-413-a |
| LP-469-a | LP-424-a |
| LP-469-a | LP-425-a |
| LP-469-a | LP-426-a |
| LP-469-a | LP-427-a |
| LP-469-a | LP-428-a |
| LP-469-a | LP-432-a |
| LP-469-a | LP-433-a |
| LP-469-a | LP-444-a |
| LP-469-a | LP-445-a |
| LP-469-a | LP-446-a |
| LP-469-a | LP-447-a |
| LP-469-a | LP-453-a |
| LP-469-a | LP-455-a |
| LP-469-a | LP-457-a |
| LP-469-a | LP-458-a |
| LP-469-a | LP-459-a |
| LP-469-a | LP-460-a |
| LP-469-a | LP-461-a |
| LP-469-a | LP-468-a |
| LP-469-a | LP-469-a |
| LP-469-a | LP-470-a |
| LP-469-a | LP-473-a |
| LP-469-a | LP-474-a |
| LP-469-a | CNR1 SM2-a |
| LP-469-a | (None) |
| LP-470-a | LP-4-a |
| LP-470-a | LP-18-a |
| LP-470-a | LP-128-a |
| LP-470-a | LP-151-a |
| LP-470-a | LP-183-a |
| LP-470-a | LP-200-a |
| LP-470-a | LP-208-a |
| LP-470-a | LP-211-a |
| LP-470-a | LP-232-a |
| LP-470-a | LP-242-a |
| LP-470-a | LP-243-a |
| LP-470-a | LP-244-a |
| LP-470-a | LP-245-a |
| LP-470-a | LP-249-a |
| LP-470-a | LP-274-a |
| LP-470-a | LP-295-a |
| LP-470-a | LP-310-a |
| LP-470-a | LP-359-a |
| LP-470-a | LP-361-a |
| LP-470-a | LP-371-a |
| LP-470-a | LP-374-a |
| LP-470-a | LP-375-a |
| LP-470-a | LP-377-a |
| LP-470-a | LP-378-a |
| LP-470-a | LP-379-a |
| LP-470-a | LP-380-a |
| LP-470-a | LP-403-a |
| LP-470-a | LP-404-a |
| LP-470-a | LP-412-a |
| LP-470-a | LP-413-a |
| LP-470-a | LP-424-a |
| LP-470-a | LP-425-a |
| LP-470-a | LP-426-a |
| LP-470-a | LP-427-a |
| LP-470-a | LP-428-a |
| LP-470-a | LP-432-a |
| LP-470-a | LP-433-a |
| LP-470-a | LP-444-a |
| LP-470-a | LP-445-a |
| LP-470-a | LP-446-a |
| LP-470-a | LP-447-a |
| LP-470-a | LP-453-a |
| LP-470-a | LP-455-a |
| LP-470-a | LP-457-a |
| LP-470-a | LP-458-a |
| LP-470-a | LP-459-a |
| LP-470-a | LP-460-a |
| LP-470-a | LP-461-a |
| LP-470-a | LP-468-a |
| LP-470-a | LP-469-a |
| LP-470-a | LP-470-a |
| LP-470-a | LP-473-a |
| LP-470-a | LP-474-a |
| LP-470-a | CNR1 SM2-a |
| LP-470-a | (None) |
| LP-473-a | LP-4-a |
| LP-473-a | LP-18-a |
| LP-473-a | LP-128-a |
| LP-473-a | LP-151-a |
| LP-473-a | LP-183-a |
| LP-473-a | LP-200-a |
| LP-473-a | LP-208-a |
| LP-473-a | LP-211-a |
| LP-473-a | LP-232-a |
| LP-473-a | LP-242-a |
| LP-473-a | LP-243-a |
| LP-473-a | LP-244-a |
| LP-473-a | LP-245-a |
| LP-473-a | LP-249-a |
| LP-473-a | LP-274-a |
| LP-473-a | LP-295-a |
| LP-473-a | LP-310-a |
| LP-473-a | LP-359-a |
| LP-473-a | LP-361-a |
| LP-473-a | LP-371-a |
| LP-473-a | LP-374-a |
| LP-473-a | LP-375-a |
| LP-473-a | LP-377-a |
| LP-473-a | LP-378-a |
| LP-473-a | LP-379-a |
| LP-473-a | LP-380-a |
| LP-473-a | LP-403-a |
| LP-473-a | LP-404-a |
| LP-473-a | LP-412-a |
| LP-473-a | LP-413-a |
| LP-473-a | LP-424-a |
| LP-473-a | LP-425-a |
| LP-473-a | LP-426-a |
| LP-473-a | LP-427-a |
| LP-473-a | LP-428-a |
| LP-473-a | LP-432-a |
| LP-473-a | LP-433-a |
| LP-473-a | LP-444-a |
| LP-473-a | LP-445-a |
| LP-473-a | LP-446-a |
| LP-473-a | LP-447-a |
| LP-473-a | LP-453-a |
| LP-473-a | LP-455-a |
| LP-473-a | LP-457-a |
| LP-473-a | LP-458-a |
| LP-473-a | LP-459-a |
| LP-473-a | LP-460-a |
| LP-473-a | LP-461-a |
| LP-473-a | LP-468-a |
| LP-473-a | LP-469-a |
| LP-473-a | LP-470-a |
| LP-473-a | LP-473-a |
| LP-473-a | LP-474-a |
| LP-473-a | CNR1 SM2-a |
| LP-473-a | (None) |
| LP-474-a | LP-4-a |
| LP-474-a | LP-18-a |
| LP-474-a | LP-128-a |
| LP-474-a | LP-151-a |
| LP-474-a | LP-183-a |
| LP-474-a | LP-200-a |
| LP-474-a | LP-208-a |
| LP-474-a | LP-211-a |
| LP-474-a | LP-232-a |
| LP-474-a | LP-242-a |
| LP-474-a | LP-243-a |
| LP-474-a | LP-244-a |
| LP-474-a | LP-245-a |
| LP-474-a | LP-249-a |
| LP-474-a | LP-274-a |
| LP-474-a | LP-295-a |
| LP-474-a | LP-310-a |
| LP-474-a | LP-359-a |
| LP-474-a | LP-361-a |
| LP-474-a | LP-371-a |
| LP-474-a | LP-374-a |
| LP-474-a | LP-375-a |
| LP-474-a | LP-377-a |
| LP-474-a | LP-378-a |
| LP-474-a | LP-379-a |
| LP-474-a | LP-380-a |
| LP-474-a | LP-403-a |
| LP-474-a | LP-404-a |
| LP-474-a | LP-412-a |
| LP-474-a | LP-413-a |
| LP-474-a | LP-424-a |
| LP-474-a | LP-425-a |
| LP-474-a | LP-426-a |
| LP-474-a | LP-427-a |
| LP-474-a | LP-428-a |
| LP-474-a | LP-432-a |
| LP-474-a | LP-433-a |
| LP-474-a | LP-444-a |
| LP-474-a | LP-445-a |
| LP-474-a | LP-446-a |
| LP-474-a | LP-447-a |
| LP-474-a | LP-453-a |
| LP-474-a | LP-455-a |
| LP-474-a | LP-457-a |
| LP-474-a | LP-458-a |
| LP-474-a | LP-459-a |
| LP-474-a | LP-460-a |
| LP-474-a | LP-461-a |
| LP-474-a | LP-468-a |
| LP-474-a | LP-469-a |
| LP-474-a | LP-470-a |
| LP-474-a | LP-473-a |
| LP-474-a | LP-474-a |
| LP-474-a | CNR1 SM2-a |
| LP-474-a | (None) |
| CNR1 SM2-a | LP-4-a |
| CNR1 SM2-a | LP-18-a |
| CNR1 SM2-a | LP-128-a |
| CNR1 SM2-a | LP-151-a |
| CNR1 SM2-a | LP-183-a |
| CNR1 SM2-a | LP-200-a |
| CNR1 SM2-a | LP-208-a |
| CNR1 SM2-a | LP-211-a |
| CNR1 SM2-a | LP-232-a |
| CNR1 SM2-a | LP-242-a |
| CNR1 SM2-a | LP-243-a |
| CNR1 SM2-a | LP-244-a |
| CNR1 SM2-a | LP-245-a |
| CNR1 SM2-a | LP-249-a |
| CNR1 SM2-a | LP-274-a |
| CNR1 SM2-a | LP-295-a |
| CNR1 SM2-a | LP-310-a |
| CNR1 SM2-a | LP-359-a |
| CNR1 SM2-a | LP-361-a |
| CNR1 SM2-a | LP-371-a |
| CNR1 SM2-a | LP-374-a |
| CNR1 SM2-a | LP-375-a |
| CNR1 SM2-a | LP-377-a |
| CNR1 SM2-a | LP-378-a |
| CNR1 SM2-a | LP-379-a |
| CNR1 SM2-a | LP-380-a |
| CNR1 SM2-a | LP-403-a |
| CNR1 SM2-a | LP-404-a |
| CNR1 SM2-a | LP-412-a |
| CNR1 SM2-a | LP-413-a |
| CNR1 SM2-a | LP-424-a |
| CNR1 SM2-a | LP-425-a |
| CNR1 SM2-a | LP-426-a |
| CNR1 SM2-a | LP-427-a |
| CNR1 SM2-a | LP-428-a |
| CNR1 SM2-a | LP-432-a |
| CNR1 SM2-a | LP-433-a |
| CNR1 SM2-a | LP-444-a |
| CNR1 SM2-a | LP-445-a |
| CNR1 SM2-a | LP-446-a |
| CNR1 SM2-a | LP-447-a |
| CNR1 SM2-a | LP-453-a |
| CNR1 SM2-a | LP-455-a |
| CNR1 SM2-a | LP-457-a |
| CNR1 SM2-a | LP-458-a |
| CNR1 SM2-a | LP-459-a |
| CNR1 SM2-a | LP-460-a |
| CNR1 SM2-a | LP-461-a |
| CNR1 SM2-a | LP-468-a |
| CNR1 SM2-a | LP-469-a |
| CNR1 SM2-a | LP-470-a |
| CNR1 SM2-a | LP-473-a |
| CNR1 SM2-a | LP-474-a |
| CNR1 SM2-a | CNR1 SM2-a |
| CNR1 SM2-a | (None) |
| (None) | LP-4-a |
| (None) | LP-18-a |
| (None) | LP-128-a |
| (None) | LP-151-a |
| (None) | LP-183-a |
| (None) | LP-200-a |
| (None) | LP-208-a |
| (None) | LP-211-a |
| (None) | LP-232-a |
| (None) | LP-242-a |
| (None) | LP-243-a |
| (None) | LP-244-a |
| (None) | LP-245-a |
| (None) | LP-249-a |
| (None) | LP-274-a |
| (None) | LP-295-a |
| (None) | LP-310-a |
| (None) | LP-359-a |
| (None) | LP-361-a |
| (None) | LP-371-a |
| (None) | LP-374-a |
| (None) | LP-375-a |
| (None) | LP-377-a |
| (None) | LP-378-a |
| (None) | LP-379-a |
| (None) | LP-380-a |
| (None) | LP-403-a |
| (None) | LP-404-a |
| (None) | LP-412-a |
| (None) | LP-413-a |
| (None) | LP-424-a |
| (None) | LP-425-a |
| (None) | LP-426-a |
| (None) | LP-427-a |
| (None) | LP-428-a |
| (None) | LP-432-a |
| (None) | LP-433-a |
| (None) | LP-444-a |
| (None) | LP-445-a |
| (None) | LP-446-a |
| (None) | LP-447-a |
| (None) | LP-453-a |
| (None) | LP-455-a |
| (None) | LP-457-a |
| (None) | LP-458-a |
| (None) | LP-459-a |
| (None) | LP-460-a |
| (None) | LP-461-a |
| (None) | LP-468-a |
| (None) | LP-469-a |
| (None) | LP-470-a |
| (None) | LP-473-a |
| (None) | LP-474-a |
| (None) | CNR1 SM2-a |

In some embodiments, lipid PK/PD modulators may comprise compounds having the formula shown in Table 3.

TABLE 3
Lipid PK/PD modulators

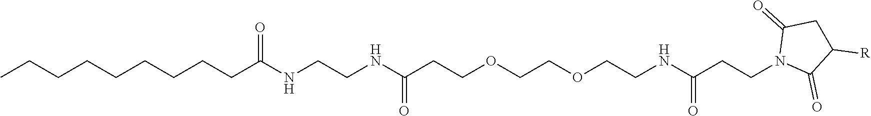

LP-4-b

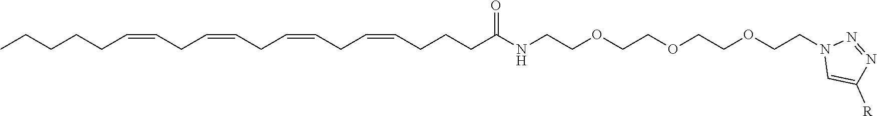

LP-18-b

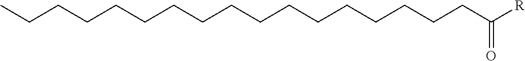

LP-128-b

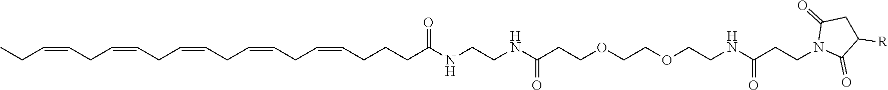

LP-151-b

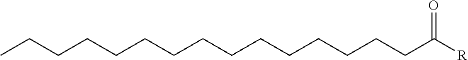

LP-183-b

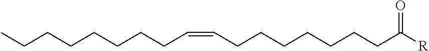

LP-200-b

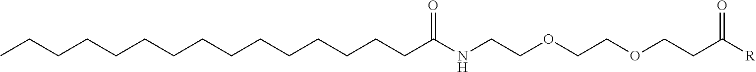

LP-208-b

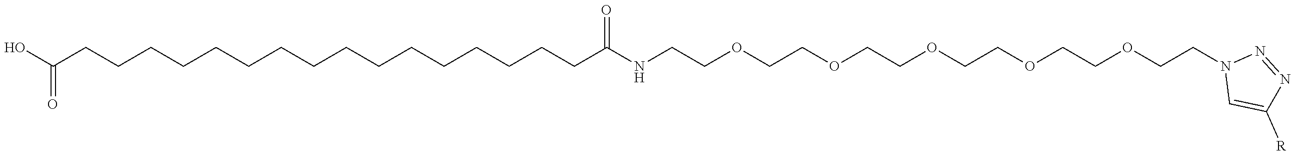

LP-211-b

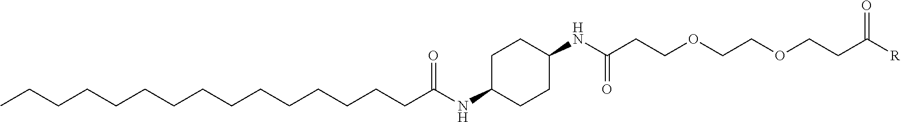

LP-232-b

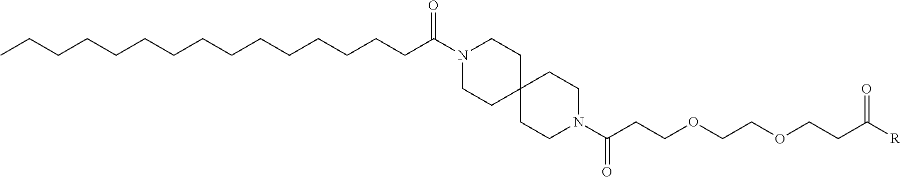

LP-242-b

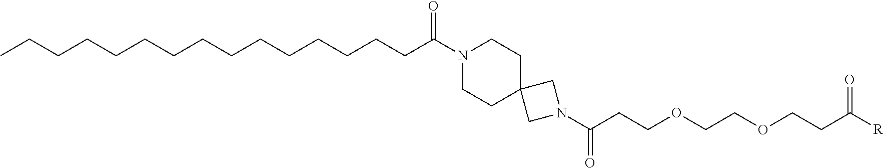

LP-243-b

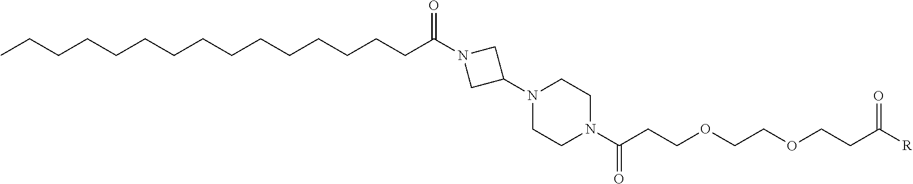

LP-244-b

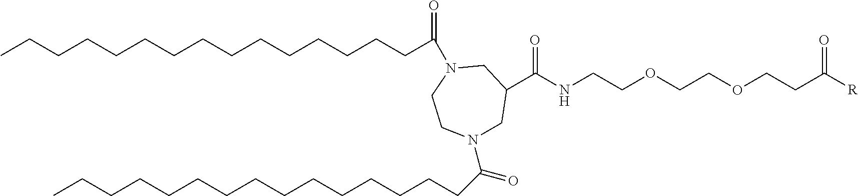

LP-245-b

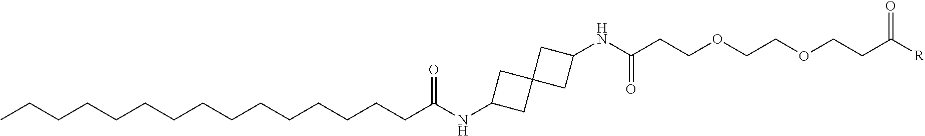

LP-249-b

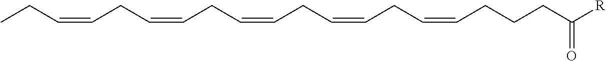

LP-274-b

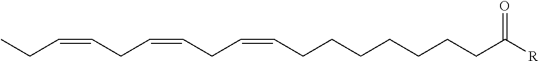

LP-295-b

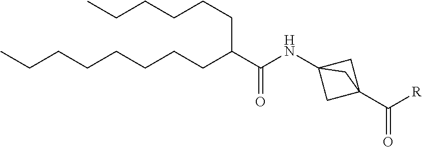

LP-310-b

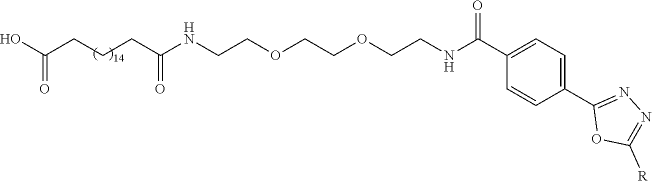

LP-359-b

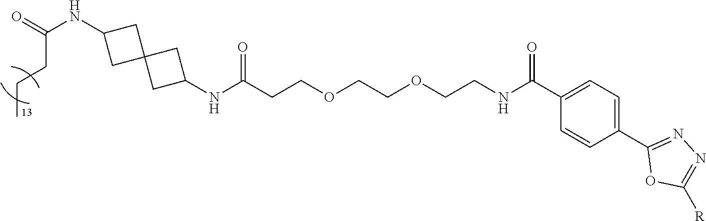

LP-361-b

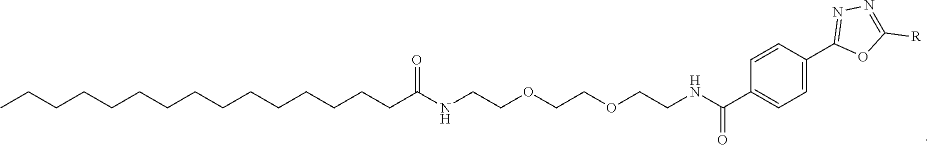

LP-371-b

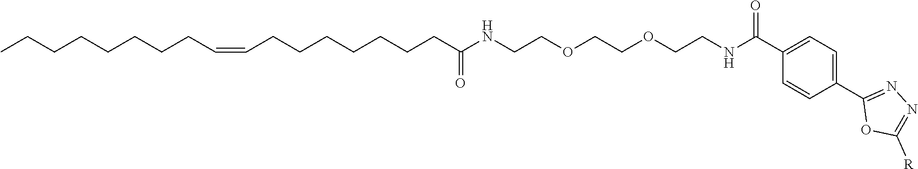

LP-374-b

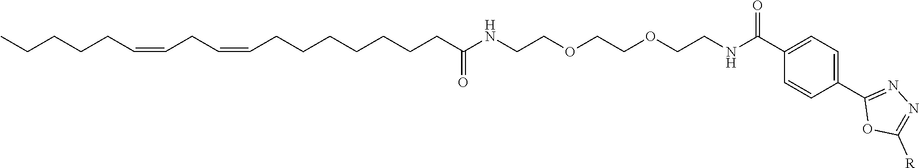

LP-375-b

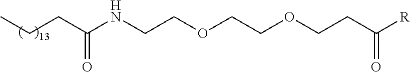

LP-377-b

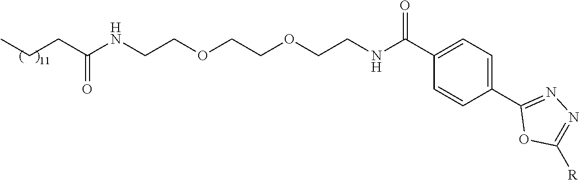

LP-378-b

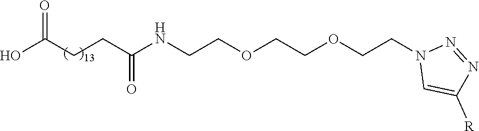

LP-379-b

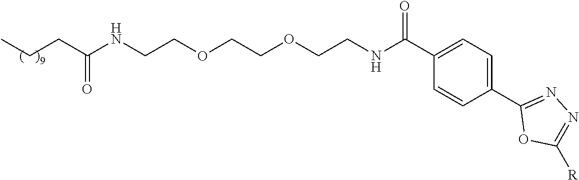

LP-380-b

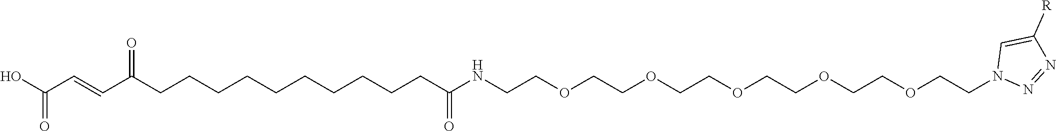

LP-403-b

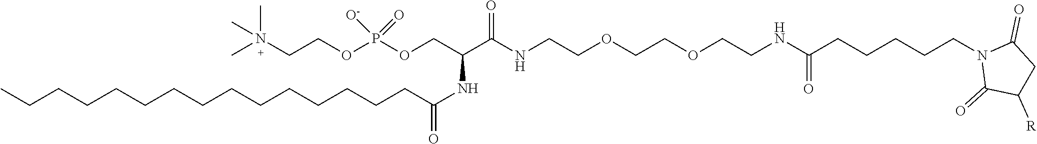

LP-404-b

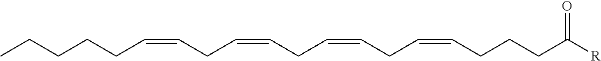

LP-412-b

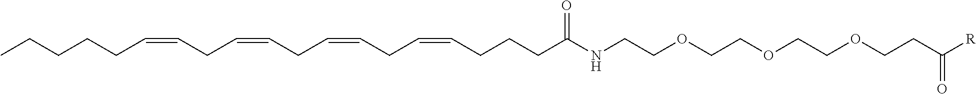

LP-413-b

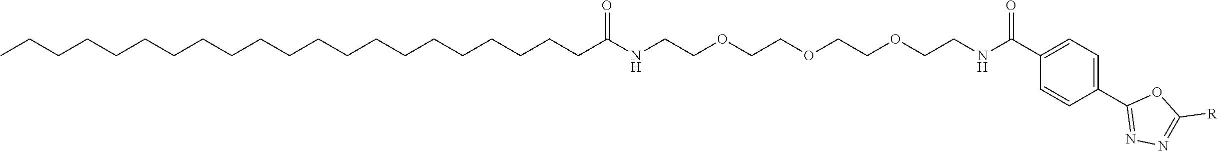

LP-416-b

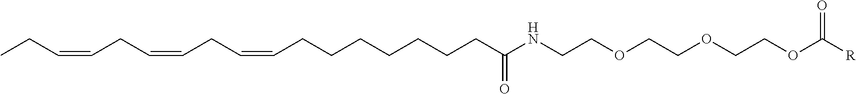

LP-424-b

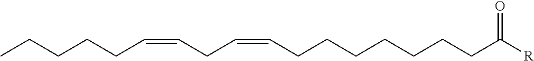

LP-425-b

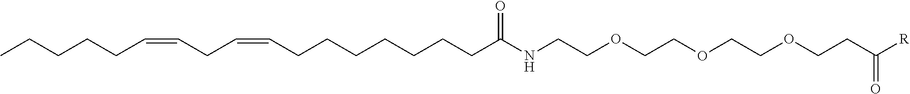

LP-426-b

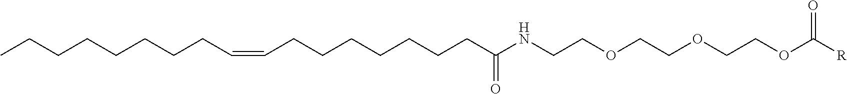

LP-427-b

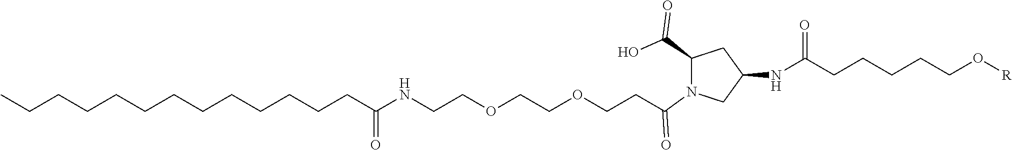

LP-428-b

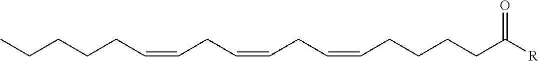

LP-432-b

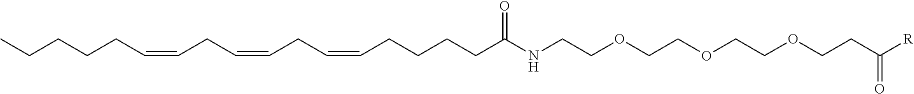

LP-433-b

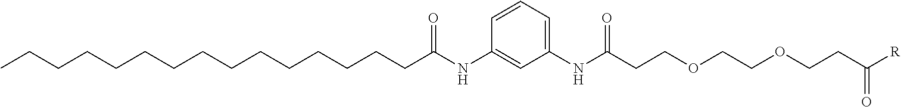

LP-444-b

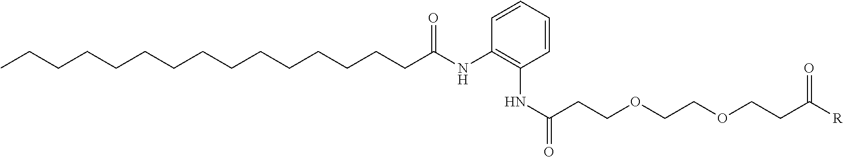

LP-445-b

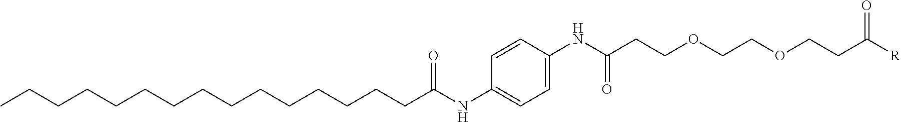

LP-446-b

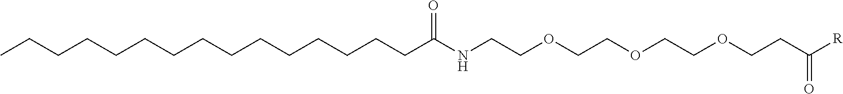

LP-447-b

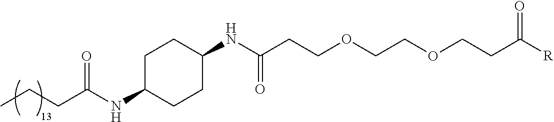

LP-453-b

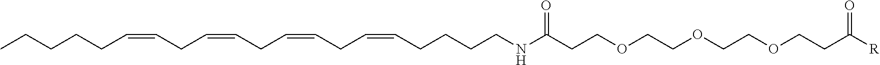

LP-455-b

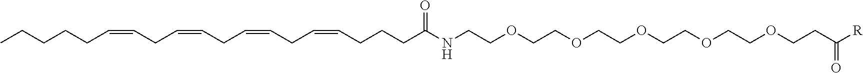

LP-457-b

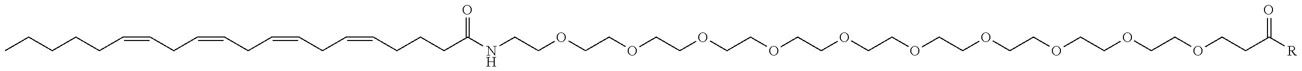

LP-458-b

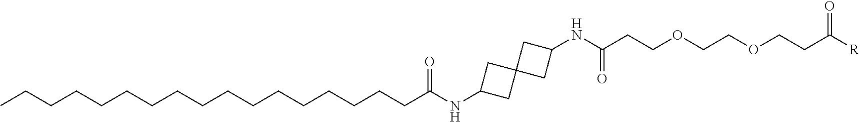

LP-459-b

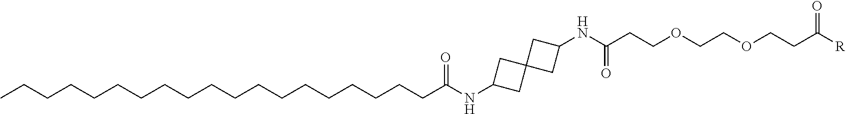

LP-460-b

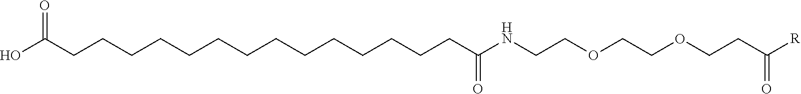

LP-461-b

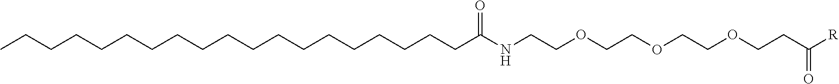

LP-468-b

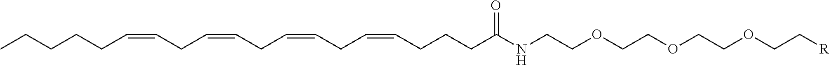

LP-469-b

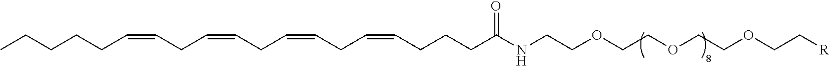

LP-470-b

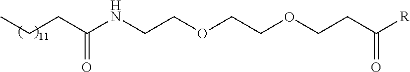

LP-473-b

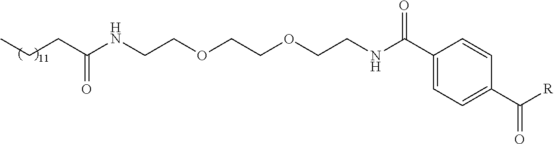

LP-474-b

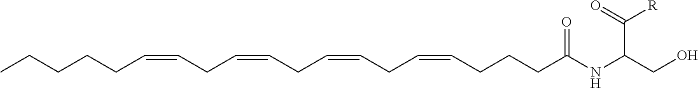

CNR1 SM2-b

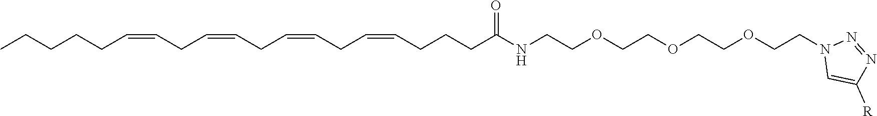

CNR1 SM2-1-b

wherein R comprises an oligonucleotide.

### Definitions

As used herein, the terms “oligonucleotide” and “polynucleotide” mean a polymer of linked nucleosides each of which can be independently modified or unmodified.

As used herein, the term “oligonucleotide-based agent” means a chemical composition comprised of one or more oligonucleotides. “Oligonucleotide-based agents” include, but are not limited to: single-stranded oligonucleotides, single-stranded antisense oligonucleotides, short interfering RNAs (siRNAs), double-strand RNAs (dsRNA), micro RNAs (miRNAs), short hairpin RNAs (shRNA), ribozymes, interfering RNA molecules, and dicer substrates. The general aspects related to the manufacture, design, and synthesis of oligonucleotide-based agents is known in the art.

As used herein, an “antisense oligonucleotide” means a single stranded oligonucleotide molecule wherein a portion of a nucleotide sequence is at least partially complementary to a messenger RNA (mRNA) such that it is capable of hybridizing and thereby blocking translation of the mRNA into protein and inhibiting expression.

As used herein, an “RNAi agent” (also referred to as an “RNAi trigger”) means a composition that contains an RNA or RNA-like (e.g., chemically modified RNA) oligonucleotide molecule that is capable of degrading or inhibiting (e.g., degrades or inhibits under appropriate conditions) translation of messenger RNA (mRNA) transcripts of a target mRNA in a sequence specific manner. As used herein, RNAi agents may operate through the RNA interference mechanism (i.e., inducing RNA interference through interaction with the RNA interference pathway machinery (RNA-induced silencing complex or RISC) of mammalian cells), or by any alternative mechanism(s) or pathway(s). While it is believed that RNAi agents, as that term is used herein, operate primarily through the RNA interference mechanism, the disclosed RNAi agents are not bound by or limited to any particular pathway or mechanism of action. RNAi agents disclosed herein are comprised of a sense strand and an antisense strand, and include, but are not limited to: short (or small) interfering RNAs (siRNAs), double stranded RNAs (dsRNA), micro RNAs (miRNAs), short hairpin RNAs (shRNA), and dicer substrates. The antisense strand of the RNAi agents described herein is at least partially complementary to the mRNA being targeted. RNAi agents can include one or more modified nucleotides and/or one or more non-phosphodiester linkages.

As used herein, the term “lipid” refers to moieties and molecules that are soluble in nonpolar solvents. The term lipid includes amphiphilic molecules comprising a polar, water-soluble head group and a hydrophobic tail. Lipids can be of natural or synthetic origin. Non-limiting examples of lipids include fatty acids (e.g., saturated fatty acids, monounsaturated fatty acids, and polyunsaturated fatty acids), glycerolipids (e.g., monoacylglycerols, diacylglycerols, and triacylglycerols), phospholipids (e.g., phosphatidylethanolamine, phosphatidylcholine, and phosphatidylserine), sphingolipids (e.g., sphingomyelin), and cholesterol esters. As used herein, the term “saturated lipid” refers to lipids that are free of any unsaturation. As used herein, the term “unsaturated lipid” refers to lipids that comprise at least one (1) degree of unsaturation. As used herein, the term “branched lipid” refers to lipids comprising more than one linear chain, wherein each liner chain is covalently attached to at least one other linear chain. As used herein, the term “straight chain lipid” refers to lipids that are free of any branching.

As used herein, the terms “silence,” “reduce,” “inhibit,” “down-regulate,” or “knockdown” when referring to expression of a given gene, mean that the expression of the gene, as measured by the level of RNA transcribed from the gene or the level of polypeptide, protein, or protein subunit translated from the mRNA in a cell, group of cells, tissue, organ, or subject in which the gene is transcribed, is reduced when the cell, group of cells, tissue, organ, or subject is treated with the oligonucleotide-based agents described herein as compared to a second cell, group of cells, tissue, organ, or subject that has not or have not been so treated.

As used herein, the terms “sequence” and “nucleotide sequence” mean a succession or order of nucleobases or nucleotides, described with a succession of letters using standard nomenclature.

As used herein, a “base,” “nucleotide base,” or “nucleobase,” is a heterocyclic pyrimidine or purine compound that is a component of a nucleotide, and includes the primary purine bases adenine and guanine, and the primary pyrimidine bases cytosine, thymine, and uracil. A nucleobase may further be modified to include, without limitation, universal bases, hydrophobic bases, promiscuous bases, size-expanded bases, and fluorinated bases. (See, e.g., Modified Nucleosides in Biochemistry, Biotechnology and Medicine, Herdewijn, P. ed. Wiley-VCH, 2008). The synthesis of such modified nucleobases (including phosphoramidite compounds that include modified nucleobases) is known in the art.

As used herein, and unless otherwise indicated, the term “complementary,” when used to describe a first nucleobase or nucleotide sequence (e.g., RNAi agent sense strand or targeted mRNA) in relation to a second nucleobase or nucleotide sequence (e.g., RNAi agent antisense strand or a single-stranded antisense oligonucleotide), means the ability of an oligonucleotide or polynucleotide including the first nucleotide sequence to hybridize (form base pair hydrogen bonds under mammalian physiological conditions (or similar conditions in vitro)) and form a duplex or double helical structure under certain standard conditions with an oligonucleotide or polynucleotide including the second nucleotide sequence. Complementary sequences include Watson-Crick base pairs or non-Watson-Crick base pairs and include natural or modified nucleotides or nucleotide mimics, at least to the extent that the above hybridization requirements are fulfilled. Sequence identity or complementarity is independent of modification. For example, a and Af, as defined herein, are complementary to U (or T) and identical to A for the purposes of determining identity or complementarity.

As used herein, “perfectly complementary” or “fully complementary” means that in a hybridized pair of nucleobase or nucleotide sequence molecules, all (100%) of the bases in a contiguous sequence of a first oligonucleotide will hybridize with the same number of bases in a contiguous sequence of a second oligonucleotide. The contiguous sequence may comprise all or a part of a first or second nucleotide sequence.

As used herein, “partially complementary” means that in a hybridized pair of nucleobase or nucleotide sequence molecules, at least 70%, but not all, of the bases in a contiguous sequence of a first oligonucleotide will hybridize with the same number of bases in a contiguous sequence of a second oligonucleotide. The contiguous sequence may comprise all or a part of a first or second nucleotide sequence.

As used herein, “substantially complementary” means that in a hybridized pair of nucleobase or nucleotide sequence molecules, at least 85%, but not all, of the bases in a contiguous sequence of a first oligonucleotide will hybridize with the same number of bases in a contiguous sequence of a second oligonucleotide. The contiguous sequence may comprise all or a part of a first or second nucleotide sequence.

As used herein, the terms “complementary,” “fully complementary,” “partially complementary,” and “substantially complementary” are used with respect to the nucleobase or nucleotide matching between the sense strand and the antisense strand of an RNAi agent, or between the antisense strand of an RNAi agent and a sequence of a target mRNA.

As used herein, the term “substantially identical” or “substantial identity,” as applied to a nucleic acid sequence means the nucleotide sequence (or a portion of a nucleotide sequence) has at least about 85% sequence identity or more, e.g., at least 90%, at least 95%, or at least 99% identity, compared to a reference sequence. Percentage of sequence identity is determined by comparing two optimally aligned sequences over a comparison window. The percentage is calculated by determining the number of positions at which the same type of nucleic acid base occurs in both sequences to yield the number of matched positions, dividing the number of matched positions by the total number of positions in the window of comparison and multiplying the result by 100 to yield the percentage of sequence identity. The inventions disclosed herein encompass nucleotide sequences substantially identical to those disclosed herein.

As used herein, the terms “treat,” “treatment,” and the like, mean the methods or steps taken to provide relief from or alleviation of the number, severity, and/or frequency of one or more symptoms of a disease in a subject. As used herein, “treat” and “treatment” may include the preventative treatment, management, prophylactic treatment, and/or inhibition or reduction of the number, severity, and/or frequency of one or more symptoms of a disease in a subject.

As used herein, the phrase “introducing into a cell,” when referring to an oligonucleotide-based agent, means functionally delivering the oligonucleotide-based agent into a cell. The phrase “functional delivery,” means delivering the oligonucleotide-based agent to the cell in a manner that enables the oligonucleotide-based agent to have the expected biological activity, e.g., sequence-specific inhibition of gene expression.

As used herein, the term “isomers” refers to compounds that have identical molecular formulae, but that differ in the nature or the sequence of bonding of their atoms or in the arrangement of their atoms in space. Isomers that differ in the arrangement of their atoms in space are termed “stereoisomers.” Stereoisomers that are not mirror images of one another are termed “diastereomers,” and stereoisomers that are non-superimposable mirror images are termed “enantiomers,” or sometimes optical isomers. A carbon atom bonded to four non-identical substituents is termed a “chiral center.”

As used herein, unless specifically identified in a structure as having a particular conformation, for each structure in which asymmetric centers are present and thus give rise to enantiomers, diastereomers, or other stereoisomeric configurations, each structure disclosed herein is intended to represent all such possible isomers, including their optically pure and racemic forms. For example, the structures disclosed herein are intended to cover mixtures of diastereomers as well as single stereoisomers.

As used in a claim herein, the phrase “consisting of” excludes any element, step, or ingredient not specified in the claim. When used in a claim herein, the phrase “consisting essentially of” limits the scope of a claim to the specified materials or steps and those that do not materially affect the basic and novel characteristic(s) of the claimed invention.

The person of ordinary skill in the art would readily understand and appreciate that the compounds and compositions disclosed herein may have certain atoms (e.g., N, O, or S atoms) in a protonated or deprotonated state, depending upon the environment in which the compound or composition is placed. Accordingly, as used herein, the structures disclosed herein envisage that certain functional groups, such as, for example, OH, SH, or NH, may be protonated or deprotonated. The disclosure herein is intended to cover the disclosed compounds and compositions regardless of their state of protonation based on the environment (such as pH), as would be readily understood by the person of ordinary skill in the art.

As used herein, the term “linked” or “conjugated” when referring to the connection between two compounds or molecules means that two molecules are joined by a covalent bond or are associated via noncovalent bonds (e.g., hydrogen bonds or ionic bonds). In some examples, where the term “linked” or “conjugated” refers to the association between two molecules via noncovalent bonds, the association between the two different molecules has a K_{D} of less than 1×10⁻⁴ M (e.g., less than 1×10⁻⁵ M, less than 1×10⁻⁶ M, or less than 1×10⁻⁷ M) in physiologically acceptable buffer (e.g., buffered saline). Unless stated, the terms “linked” and “conjugated” as used herein may refer to the connection between a first compound and a second compound either with or without any intervening atoms or groups of atoms.

As used herein, a linking group is one or more atoms that connects one molecule or portion of a molecule to another to second molecule or second portion of a molecule. Similarly, as used in the art, the term scaffold is sometimes used interchangeably with a linking group. Linking groups may comprise any number of atoms or functional groups. In some embodiments, linking groups may not facilitate any biological or pharmaceutical response, and merely serve to link two biologically active molecules.

As used herein, the term “alkyl” refers to a saturated aliphatic hydrocarbon group containing 1-12 (e.g., 1-8, 1-6, 1-4, or 1-3) carbon atoms. An alkyl group can be straight or branched. Examples of alkyl groups include, but are not limited to, methyl, ethyl, propyl, isopropyl, butyl, isobutyl, sec-butyl, tert-butyl, n-pentyl, n-heptyl, or 2-ethylhexyl.

The term “alkenyl” refers to a straight-chain or branched hydrocarbon group having from 2 to 10 carbon atoms and one or more carbon-carbon double bonds (e.g., 1, 2, 3, or 4 carbon-carbon double bonds). In an alkenyl group, a C═C double bond for which the stereochemistry is not specified (e.g., —CH═CHCH₃) may be an (E)- or (Z)-double bond.

The term “alkynyl” refers to a straight-chain or branched hydrocarbon group having from 2 to 10 carbon atoms and one or more carbon-carbon triple bonds (e.g., 1, 2, 3, or 4 triple bonds).

The term “carbocyclyl” or “carbocyclic” refers to a non-aromatic cyclic hydrocarbon group having from 3 to 14 ring carbon atoms (“C₃₋₁₄ carbocyclyl”) and zero heteroatoms in the ring system. The carbocyclyl group can be monocyclic (“monocyclic carbocyclyl”) or polycyclic (e.g., containing a fused, bridged or spiro ring system) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system.

The term “heterocyclyl” or “heterocyclic” refers to a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system, and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system.

The term “aryl” refers to a monocyclic or polycyclic (e.g., bicyclic or tricyclic) 4n+2 aromatic ring system (e.g., having 6, 10, or 14 pi electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C6 aryl”; e.g., phenyl). In some embodiments, an aryl group has 10 ring carbon atoms (“C10 aryl”; e.g., naphthyl such as 1-naphthyl and 2-naphthyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system.

The term “heteroaryl” refers to a radical of a 5-14 membered monocyclic or polycyclic (e.g., bicyclic) 4n+2 aromatic ring system (e.g., having 6, 10, or 14 pi electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

Unless stated otherwise, use of the symbol

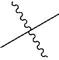

as used herein means that any group or groups may be linked (or connected) thereto that is in accordance with the scope of the inventions described herein.

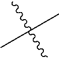

In instances where the symbol is used twice in a structure, it is to be understood that the structure is bivalent, and is connected to two other groups. In some embodiments, when chemically feasible, a bivalent structure may be oriented such that the structure may be attached in either direction, e.g., a structure such as

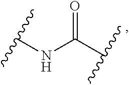 [SMILES: *NC(*)=O]

may also be read as

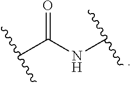 [SMILES: *NC(*)=O]

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

As used herein, the term “including” is used to herein mean, and is used interchangeably with, the phrase “including but not limited to.” The term “or” is used herein to mean, and is used interchangeably with, the term “and/or,” unless the context clearly indicates otherwise.

As used in a claim herein, the phrase “consisting of” excludes any element, step, or ingredient not specified in the claim. When used in a claim herein, the phrase “consisting essentially of” limits the scope of a claim to the specified materials or steps and those that do not materially affect the basic and novel characteristic(s) of the claimed invention.

### Oligonucleotide-Based Agents, Including RNAi Agents

As used herein, an “oligonucleotide-based agent” is a composition that comprises at least one nucleotide sequence containing about 8-50 (e.g., 8 to 48, 8 to 46, 8 to 44, 8 to 42, 8 to 40, 8 to 38, 8 to 36, 8 to 34, 8 to 32, 8 to 30, 8 to 28, 8 to 26, 8 to 24, 8 to 22, 8 to 20, 8 to 18, 8 to 16, 8 to 14, 8 to 12, 8 to 10, 10 to 48, 10 to 46, 10 to 44, 10 to 42, 10 to 40, 10 to 38, 10 to 36, 10 to 34, 10 to 32, 10 to 30, 10 to 28, 10 to 26, 10 to 24, 10 to 22, 10 to 20, 10 to 18, 10 to 16, 10 to 14, 10 to 12, 12 to 50, 12 to 48, 12 to 46, 12 to 44, 12 to 42, 12 to 40, 12 to 38, 12 to 36, 12 to 34, 12 to 32, 12 to 30, 12 to 28, 12 to 26, 12 to 24, 12 to 22, 12 to 20, 12 to 18, 12 to 16, 12 to 14, 14 to 50, 14 to 48, 14 to 46, 14 to 44, 14 to 42, 14 to 40, 14 to 38, 14 to 36, 14 to 34, 14 to 32, 14 to 30, 14 to 28, 14 to 26, 14 to 24, 14 to 22, 14 to 20, 14 to 18, 14 to 16, 16 to 50, 16 to 48, 16 to 46, 16 to 44, 16 to 42, 16 to 40, 16 to 38, 16 to 36, 16 to 34, 16 to 32, 16 to 30, 16 to 28, 16 to 26, 16 to 24, 16 to 22, 16 to 20, 16 to 18, 18 to 50, 18 to 48, 18 to 46, 18 to 44, 18 to 42, 18 to 40, 18 to 38, 18 to 36, 18 to 34, 18 to 32, 18 to 30, 18 to 28, 18 to 26, 18 to 24, 18 to 22, 18 to 20, 20 to 50, 20 to 48, 20 to 46, 20 to 44, 20 to 42, 20 to 40, 20 to 38, 20 to 36, 20 to 34, 20 to 32, 20 to 30, 20 to 28, 20 to 26, 20 to 24, 20 to 22, 22 to 50, 22 to 48, 22 to 46, 22 to 44, 22 to 42, 22 to 40, 22 to 38, 22 to 36, 22 to 34, 22 to 32, 22 to 30, 22 to 28, 22 to 26, 22 to 24, 24 to 50, 24 to 48, 24 to 46, 24 to 44, 24 to 42, 24 to 40, 24 to 38, 24 to 36, 24 to 34, 24 to 32, 24 to 30, 24 to 28, 24 to 26, 26 to 50, 26 to 48, 26 to 46, 26 to 44, 26 to 42, 26 to 40, 26 to 38, 26 to 36, 26 to 34, 26 to 32, 26 to 30, 26 to 28, 28 to 50, 28 to 48, 28 to 46, 28 to 44, 28 to 42, 28 to 40, 28 to 38, 28 to 36, 28 to 34, 28 to 32, to 28 to 30, 30 to 50, 30 to 48, 30 to 46, 30 to 44, 30 to 42, 30 to 40, 30 to 38, 30 to 36, 30 to 34, 30 to 32, 32 to 50, 32 to 48, 32 to 46, 32 to 44, 32 to 42, 32 to 40, 32 to 38, 32 to 36, 32 to 34, 34 to 50, 34 to 48, 34 to 46, 34 to 44, 34 to 42, 34 to 40, 34 to 38, 34 to 36, 36 to 50, 36 to 48, 36 to 46, 36 to 44, 36 to 42, 36 to 40, 36 to 38, 38 to 50, 38 to 48, 38 to 46, 38 to 44, 38 to 42, 38 to 40, 40 to 50, 40 to 48, 40 to 46, 40 to 44, 40 to 42, 42 to 50, 42 to 48, 42 to 46, 42 to 44, 44 to 50, 44 to 48, 44 to 46, 46 to 50, 46 to 48, or 48 to 50) nucleotides or nucleotide base pairs. In some embodiments, an oligonucleotide-based agent has a nucleobase sequence that is at least partially complementary to a coding sequence in an expressed target nucleic acid or target gene within a cell. In some embodiments, the oligonucleotide-based agents, upon delivery to a cell expressing a gene, are able to inhibit the expression of the underlying gene (e.g., ALK7, Adipoq), and are referred to herein as “expression-inhibiting oligonucleotide-based agents.” The gene expression can be inhibited in vitro or in vivo.

In some embodiments, an oligonucleotide-based agent is a single-stranded oligonucleotide, such as an antisense oligonucleotide. In some embodiments, an oligonucleotide-based agent is a double-stranded oligonucleotide, such as an RNAi agent like an siRNA. In some embodiments, an oligonucleotide-based agent is a double-stranded oligonucleotide is an RNAi agent.

In some embodiments, the oligonucleotide-based agent is/are an RNAi agent. Typically, RNAi agents can be comprised of at least a sense strand (also referred to as a passenger strand) that includes a first sequence, and an antisense strand (also referred to as a guide strand) that includes a second sequence. The length of an RNAi agent sense strand can be 15 to 49 nucleotides in length and the length of the antisense strand can be 17 to 49 nucleotides in length. In some embodiments, the sense and antisense strands of an RNAi agent are independently 17 to 26 nucleotides in length. In some embodiments, the sense and antisense strands are independently 19 to 26 nucleotides in length. In some embodiments, the sense and antisense strands are independently 21 to 26 nucleotides in length. In some embodiments, the sense and antisense strands are independently 21 to 24 nucleotides in length. In some embodiments, the sense strand is 19 nucleotides in length and the antisense strand is 19 nucleotides in length. In some embodiments, the sense strand is 21 nucleotides in length and the antisense strand is 21 nucleotides in length. In some embodiments, the sense strand is 21 nucleotides in length and the antisense strand is 23 nucleotides in length. The sense and antisense strands can be either the same length or different lengths. The RNAi agents include an antisense strand sequence that is at least partially complementary to a sequence in the target gene (e.g., ALK7, Adipoq), and upon delivery to a cell expressing the target, an RNAi agent may inhibit the expression of one or more target genes in vivo or in vitro.

Oligonucleotide-based agents generally, and RNAi agents specifically, may be comprised of modified nucleotides and/or one or more non-phosphodiester linkages. As used herein, a “modified nucleotide” is a nucleotide other than a ribonucleotide (2′-hydroxyl nucleotide). In some embodiments, at least 50% (e.g., at least 60%, at least 70%, at least 80%, at least 90%, at least 95%, at least 97%, at least 98%, at least 99%, or 100%) of the nucleotides are modified nucleotides. As used herein, modified nucleotides include, but are not limited to, deoxyribonucleotides, nucleotide mimics, abasic nucleotides, 2′-modified nucleotides, 3′ to 3′ linkages (inverted) nucleotides, non-natural base-comprising nucleotides, bridged nucleotides, peptide nucleic acids, 2′,3′-seco nucleotide mimics (unlocked nucleobase analogues, locked nucleotides, 3′-O-methoxy (2′ internucleoside linked) nucleotides, 2′-F-Arabino nucleotides, 5′-Me, 2′-fluoro nucleotide, morpholino nucleotides, vinyl phosphonate deoxyribonucleotides, vinyl phosphonate containing nucleotides, and cyclopropyl phosphonate containing nucleotides. 2′-modified nucleotides (i.e. a nucleotide with a group other than a hydroxyl group at the 2′ position of the five-membered sugar ring) include, but are not limited to, 2′-O-methyl nucleotides, 2-fluoro nucleotides (alternatively referred to herein and in the art as 2′-deoxy-2′-fluoro nucleotides), 2′-deoxy nucleotides, 2′-methoxyethyl (2′-O-2-methoxylethyl) nucleotides, 2′-amino nucleotides, and 2′-alkyl nucleotides.

Moreover, one or more nucleotides of an oligonucleotide-based agent, such as an RNAi agent, may be linked by non-standard linkages or backbones (i.e., modified internucleoside linkages or modified backbones). A modified internucleoside linkage may be a non-phosphate-containing covalent internucleoside linkage. Modified internucleoside linkages or backbones include, but are not limited to, 5′-phosphorothioate groups, chiral phosphorothioates, thiophosphates, phosphorodithioates, phosphotriesters, aminoalkyl-phosphotriesters, alkyl phosphonates (e.g., methyl phosphonates or 3′-alkylene phosphonates), chiral phosphonates, phosphinates, phosphoramidates (e.g., 3′-amino phosphoramidate, aminoalkylphosphoramidates, or thionophosphoramidates), thionoalkyl-phosphonates, thionoalkylphosphotriesters, morpholino linkages, boranophosphates having normal 3′-5′ linkages, 2′-5′ linked analogs of boranophosphates, or boranophosphates having inverted polarity wherein the adjacent pairs of nucleoside units are linked 3′-5′ to 5′-3′ or 2′-5′ to 5′-2′.

It is not necessary for all positions in a given compound to be uniformly modified. Conversely, more than one modification may be incorporated in a single oligonucleotide-based agent or even in a single nucleotide thereof.

The RNAi agent sense strands and antisense strands may be synthesized and/or modified by methods known in the art. Additional disclosures related to RNAi agents may be found, for example, in the disclosure of modifications may be found, for example, in International Patent Application No. PCT/US2017/045446 (WO2018027106) to Arrowhead Pharmaceuticals, Inc., which also is incorporated by reference herein in its entirety.

### Modified Nucleotides

In some embodiments, an oligonucleotide-based agent contains one or more modified nucleotides. As used herein, a “modified nucleotide” is a nucleotide other than a ribonucleotide (2′-hydroxyl nucleotide). In some embodiments, at least 50% (e.g., at least 60%, at least 70%, at least 80%, at least 90%, at least 95%, at least 97%, at least 98%, at least 99%, or 100%) of the nucleotides are modified nucleotides. As used herein, modified nucleotides can include, but are not limited to, deoxyribonucleotides, nucleotide mimics, abasic nucleotides (represented herein as Ab), 2′-modified nucleotides, 3′ to 3′ linkages (inverted) nucleotides (represented herein as invdN, invN, invn), modified nucleobase-comprising nucleotides, bridged nucleotides, peptide nucleic acids (PNAs), 2′,3′-seco nucleotide mimics (unlocked nucleobase analogues, represented herein as NUNA or NUNA), locked nucleotides (represented herein as NLNA or NLNA), 3′-O-methoxy (2′ internucleoside linked) nucleotides (represented herein as 3′-Omen), 2′-F-Arabino nucleotides (represented herein as NfANA or Nf_{ANA}), 5′-Me, 2′-fluoro nucleotide (represented herein as 5Me-Nf), morpholino nucleotides, vinyl phosphonate deoxyribonucleotides (represented herein as vpdN), vinyl phosphonate-containing nucleotides, and cyclopropyl phosphonate-containing nucleotides (cPrpN). 2′-modified nucleotides (i.e., a nucleotide with a group other than a hydroxyl group at the 2′ position of the five-membered sugar ring) include, but are not limited to, 2′-O-methyl nucleotides (represented herein as a lower case letter ‘n’ in a nucleotide sequence), 2′-deoxy-2′-fluoro nucleotides (also referred to herein as 2′-fluoro nucleotide, and represented herein as Nf), 2′-deoxy nucleotides (represented herein as dN), 2′-methoxyethyl (2′-O-2-methoxylethyl) nucleotides (also referred to herein as 2′-MOE, and represented herein as NM), 2′-amino nucleotides, and 2′-alkyl nucleotides. It is not necessary for all positions in a given compound to be uniformly modified. Conversely, more than one modification can be incorporated in a single oligonucleotide-based agent or even in a single nucleotide thereof. The oligonucleotide-based agents can be synthesized and/or modified by methods known in the art. Modification at one nucleotide is independent of modification at another nucleotide.

Modified nucleobases include synthetic and natural nucleobases, such as 5-substituted pyrimidines, 6-azapyrimidines and N-2, N-6 and 0-6 substituted purines, (e.g., 2-aminopropyladenine, 5-propynyluracil, or 5-propynylcytosine), 5-methylcytosine (5-me-C), 5-hydroxymethyl cytosine, inosine, xanthine, hypoxanthine, 2-aminoadenine, 6-alkyl (e.g., 6-methyl, 6-ethyl, 6-isopropyl, or 6-n-butyl) derivatives of adenine and guanine, 2-alkyl (e.g., 2-methyl, 2-ethyl, 2-isopropyl, or 2-n-butyl) and other alkyl derivatives of adenine and guanine, 2-thiouracil, 2-thiothymine, 2-thiocytosine, 5-halouracil, cytosine, 5-propynyl uracil, 5-propynyl cytosine, 6-azo uracil, 6-azo cytosine, 6-azo thymine, 5-uracil (pseudouracil), 4-thiouracil, 8-halo, 8-amino, 8-sulfhydryl, 8-thioalkyl, 8-hydroxyl and other 8-substituted adenines and guanines, 5-halo (e.g., 5-bromo), 5-trifluoromethyl, and other 5-substituted uracils and cytosines, 7-methylguanine and 7-methyladenine, 8-azaguanine and 8-azaadenine, 7-deazaguanine, 7-deazaadenine, 3-deazaguanine, and 3-deazaadenine.

In some embodiments, all or substantially all of the nucleotides of an oligonucleotide-based agent, such as an RNAi agent, are modified nucleotides. As used herein, an oligonucleotide-based agent wherein substantially all of the nucleotides present are modified nucleotides is an agent having two or fewer (i.e., 0, 1, or 2) nucleotides in each oligonucleotide strand that is present being ribonucleotides (i.e., unmodified). As used herein, when the oligonucleotide-based agent is an RNAi agent such as an siRNA or double-stranded RNA, an RNAi agent wherein substantially all of the nucleotides present are modified nucleotides is an RNAi agent wherein (i) the sense strand has two or fewer (i.e., 0, 1, or 2) nucleotides in the sense strand being unmodified ribonucleotides, and (ii) the antisense strand has two or fewer (i.e., 0, 1, or 2) nucleotides being unmodified ribonucleotides. In some embodiments, one or more nucleotides of an oligonucleotide-based agent is an unmodified ribonucleotide.

### Modified Internucleoside Linkages

In some embodiments, one or more nucleotides of an oligonucleotide-based agent are linked by non-standard linkages or backbones (i.e., modified internucleoside linkages or modified backbones). Modified internucleoside linkages or backbones include, but are not limited to, phosphorothioate groups (represented herein as a lower case “s”), chiral phosphorothioates, thiophosphates, phosphorodithioates, phosphotriesters, aminoalkyl-phosphotriesters, alkyl phosphonates (e.g., methyl phosphonates or 3′-alkylene phosphonates), chiral phosphonates, phosphinates, phosphoramidates (e.g., 3′-amino phosphoramidate, aminoalkylphosphoramidates, or thionophosphoramidates), thionoalkyl-phosphonates, thionoalkylphosphotriesters, morpholino linkages, boranophosphates having normal 3′-5′ linkages, 2′-5′ linked analogs of boranophosphates, or boranophosphates having inverted polarity wherein the adjacent pairs of nucleoside units are linked 3′-5′ to 5′-3′ or 2′-5′ to 5′-2′. In some embodiments, a modified internucleoside linkage or backbone lacks a phosphorus atom. Modified internucleoside linkages lacking a phosphorus atom include, but are not limited to, short chain alkyl or cycloalkyl inter-sugar linkages, mixed heteroatom and alkyl or cycloalkyl inter-sugar linkages, or one or more short chain heteroatomic or heterocyclic inter-sugar linkages. In some embodiments, modified internucleoside backbones include, but are not limited to, siloxane backbones, sulfide backbones, sulfoxide backbones, sulfone backbones, formacetyl and thioformacetyl backbones, methylene formacetyl and thioformacetyl backbones, alkene-containing backbones, sulfamate backbones, methyleneimino and methylenehydrazino backbones, sulfonate and sulfonamide backbones, amide backbones, and other backbones having mixed N, O, S, and CH₂ components.

In some embodiments, the oligonucleotide-based agent contains one or more phosphorothioate and/or phosphorodithioate linkages. In some embodiments, all of the nucleotides of the oligonucleotide-based agent are phosphorothioate and/or phosphorodithioate linkages. In some embodiments, only the terminal 1, 2, 3, 4, or 5 nucleotides on each end of the oligonucleotide-based agent are phosphorothioate or phosphorodithioate linkages.

In some embodiments, the oligonucleotide-based agent is an RNAi agent, and in some embodiments a sense strand of an RNAi agent can contain 1, 2, 3, 4, 5, or 6 phosphorothioate or phosphorodithioate linkages, an antisense strand of an RNAi agent can contain 1, 2, 3, 4, 5, or 6 phosphorothioate or phosphorodithioate linkages, or both the sense strand and the antisense strand independently can contain 1, 2, 3, 4, 5, or 6 phosphorothioate or phosphorodithioate linkages. In some embodiments, a sense strand of an RNAi agent can contain 1, 2, 3, or 4 phosphorothioate linkages, an antisense strand of an RNAi agent can contain 1, 2, 3, or 4 phosphorothioate or phosphorodithioate linkages, or both the sense strand and the antisense strand independently can contain 1, 2, 3, or 4 phosphorothioate or phosphorodithioate linkages. In some embodiments, an RNAi agent sense strand contains at least two phosphorothioate or phosphorodithioate internucleoside linkages. In some embodiments, the at least two phosphorothioate or phosphorodithioate internucleoside linkages are between the nucleotides at positions 1-3 from the 3′ end of the sense strand. In some embodiments, one phosphorothioate internucleoside linkage is at the 5′ end of the sense strand, and another phosphorothioate linkage is at the 3′ end of the sense strand. In some embodiments, two phosphorothioate internucleoside linkage are located at the 5′ end of the sense strand, and another phosphorothioate linkage is at the 3′ end of the sense strand. In some embodiments, the sense strand does not include any phosphorothioate internucleoside linkages between the nucleotides, but contains one, two, or three phosphorothioate linkages between the terminal nucleotides on both the 5′ and 3′ ends and the optionally present inverted abasic residue terminal caps. In some embodiments, a targeting ligand or PK/PD modifier is linked to the sense strand via a phosphorothioate linkage. In some embodiments, an RNAi agent antisense strand contains four phosphorothioate internucleoside linkages. In some embodiments, the four phosphorothioate internucleoside linkages are between the nucleotides at positions 1-3 from the 5′ end of the antisense strand and between the nucleotides at positions 19-21, 20-22, 21-23, 22-24, 23-25, or 24-26 from the 5′ end. In some embodiments, three phosphorothioate internucleoside linkages are located between positions 1-4 from the 5′ end of the antisense strand, and a fourth phosphorothioate internucleoside linkage is located between positions 20-21 from the 5′ end of the antisense strand. In some embodiments, an RNAi agent contains at least three or four phosphorothioate internucleoside linkages in the antisense strand. In some embodiments, an RNAi agent contains one or more modified nucleotides and one or more modified internucleoside linkages. In some embodiments, a 2′-modified nucleoside is combined with modified internucleoside linkage.

### Capping Residues or Moieties

In some embodiments, the sense strand of an oligonucleotide or RNAi agent as described herein may include one or more capping residues or moieties, sometimes referred to in the art as a “cap,” a “terminal cap,” or a “capping residue.” As used herein, a “capping residue” is a non-nucleotide compound or other moiety that can be incorporated at one or more termini of a nucleotide sequence of an RNAi agent disclosed herein. A capping residue can provide the RNAi agent, in some instances, with certain beneficial properties, such as, for example, protection against exonuclease degradation. In some embodiments, inverted abasic residues (invAb) (also referred to in the art as “inverted abasic sites”) are added as capping residues (see Table 4). (See, e.g., F. Czaudema, Nucleic Acids Res., 2003, 31(11), 2705-16). Capping residues are generally known in the art, and include, for example, inverted abasic residues as well as carbon chains such as a terminal C₃H₇ (propyl), C₆H₁₃ (hexyl), or C₁₂H₂₅ (dodecyl) groups. In some embodiments, a capping residue is present at either the 5′ terminal end, the 3′ terminal end, or both the 5′ and 3′ terminal ends of the sense strand. In some embodiments, the 5′ end and/or the 3′ end of the sense strand may include more than one inverted abasic deoxyribose moiety as a capping residue.

In some embodiments, one or more inverted abasic residues (invAb) are added to the 3′ end of the sense strand. In some embodiments, one or more inverted abasic residues (invAb) are added to the 5′ end of the sense strand. In some embodiments, one or more inverted abasic residues or inverted abasic sites are inserted between a or PK/PD modifier and the nucleotide sequence of the sense strand of the RNAi agent. In some embodiments, the inclusion of one or more inverted abasic residues or inverted abasic sites at or near the terminal end or terminal ends of the sense strand of an RNAi agent allows for enhanced activity or other desired properties of an RNAi agent.

In some embodiments, one or more inverted abasic residues (invAb) are added to the 5′ end of the sense strand. In some embodiments, one or more inverted abasic residues can be inserted between a or PK/PD modifier and the nucleotide sequence of the sense strand of the RNAi agent. The inverted abasic residues may be linked via phosphate, phosphorothioate (e.g., shown herein as (invAb)s) (see Table 4)), or other internucleoside linkages. In some embodiments, the inclusion of one or more inverted abasic residues at or near the terminal end or terminal ends of the sense strand of an RNAi agent may allow for enhanced activity or other desired properties of an RNAi agent. In some embodiments, an inverted abasic (deoxyribose) residue can be replaced with an inverted ribitol (abasic ribose) residue. In some embodiments, the 3′ end of the antisense strand core stretch sequence, or the 3′ end of the antisense strand sequence, may include an inverted abasic residue. The chemical structures for inverted abasic deoxyribose residues are shown in Table 4.

### Linking Groups and Other Delivery Moieties

As described herein, the oligonucleotide-based agent, such as an RNAi agent, contains or is conjugated to one or more non-nucleotide groups including, but not limited to a lipid PK/PD modifier, a linking group, or another type of targeting or delivery moiety. The non-nucleotide group can enhance targeting, delivery, or attachment of the oligonucleotide-based agent. Examples of linking groups are provided in Table 4. The non-nucleotide group can be covalently linked to the 3′ and/or 5′ end of either the sense strand and/or the antisense strand. In some embodiments, an RNAi agent contains a non-nucleotide group linked to the 3′ and/or 5′ end of the sense strand. In some embodiments, a non-nucleotide group is linked to the 5′ end of an RNAi agent sense strand. A non-nucleotide group can be linked directly or indirectly to the RNAi agent via a linker/linking group. In some embodiments, a non-nucleotide group is linked to the RNAi agent via a labile, cleavable, or reversible bond or linker.

In some embodiments, a non-nucleotide group enhances the pharmacokinetic or biodistribution properties of an RNAi agent or conjugate to which it is attached to improve cell- or tissue-specific distribution and cell-specific uptake of the conjugate. In some embodiments, a non-nucleotide group enhances endocytosis of the RNAi agent.

The RNAi agents described herein can be synthesized having a reactive group, such as an amino group (also referred to herein as an amine), at the 5′-terminus and/or the 3′-terminus. The reactive group can be used subsequently to attach a targeting moiety using methods typical in the art.

For example, in some embodiments, the RNAi agents disclosed herein are synthesized having an NH₂—C₆ group at the 5′-terminus of the sense strand of the RNAi agent. The terminal amino group subsequently can be reacted to form a conjugate with, for example, a group that includes a compound having affinity for one or more integrins (i.e., and integrin targeting ligand) or a PK enhancer. In some embodiments, the RNAi agents disclosed herein are synthesized having one or more alkyne groups at the 5′-terminus of the sense strand of the RNAi agent. The terminal alkyne group(s) can subsequently be reacted to form a conjugate with, for example, a group that includes a targeting ligand.

In some embodiments, the RNAi agent is synthesized having present a linking group, which can then facilitate covalent linkage of the RNAi agent to a targeting ligand, a targeting group, a PK/PD modulator, or another type of delivery agent. The linking group can be linked to the 3′ and/or the 5′ end of the RNAi agent sense strand or antisense strand. In some embodiments, the linking group is linked to the RNAi agent sense strand. In some embodiments, the linking group is conjugated to the 5′ or 3′ end of an RNAi agent sense strand. In some embodiments, a linking group is conjugated to the 5′ end of an RNAi agent sense strand. Examples of linking groups, include, but are not limited to: C6-SS-Alk-Me, reactive groups such a primary amines and alkynes, alkyl groups, abasic residues/nucleotides, amino acids, trialkyne functionalized groups, ribitol, and/or PEG groups.

A linker or linking group is a connection between two atoms that links one chemical group (such as an RNAi agent) or segment of interest to another chemical group (such as a targeting ligand, targeting group, PK/PD modulator, or delivery agent) or segment of interest via one or more covalent bonds. A labile linkage contains a labile bond. A linkage can optionally include a spacer that increases the distance between the two joined atoms. A spacer may further add flexibility and/or length to the linkage. Spacers include, but are not limited to, alkyl groups, alkenyl groups, alkynyl groups, aryl groups, aralkyl groups, aralkenyl groups, and aralkynyl groups; each of which can contain one or more heteroatoms, heterocycles, amino acids, nucleotides, and saccharides. Spacer groups are well known in the art and the preceding list is not meant to limit the scope of the description.

In some embodiments, targeting groups are linked to the RNAi agents without the use of an additional linker. In some embodiments, the targeting group is designed to have a linker readily present to facilitate the linkage to an RNAi agent. In some embodiments, when two or more RNAi agents are included in a composition, the two or more RNAi agents can be linked to their respective targeting groups using the same linkers. In some embodiments, when two or more RNAi agents are included in a composition, the two or more RNAi agents are linked to their respective targeting groups using different linkers.

RNAi agents whether modified or unmodified, may contain 3′ and/or 5′ targeting group(s), linking group(s), and/or may be conjugated with, or comprise, PK/PD modulator(s). Any of the RNAi agent sequences or are otherwise described herein, which contain a 3′ or 5′ targeting ligand, targeting group, PK/PD modulator, or linking group, can alternatively contain no 3′ or 5′ targeting ligand, targeting group, linking group, or PK/PD modulator, or can contain a different 3′ or 5′ targeting ligand, targeting group, linking group, or PK/PD modulator including, but not limited to, those depicted in Tables 2 and 3. Any of the RNAi agent duplexes listed in Table A, whether modified or unmodified, can further comprise a targeting ligand, targeting group, linking group, or PK/PD modulator, and the targeting group or linking group can be attached to the 3′ or 5′ terminus of either the sense strand or the antisense strand of the RNAi agent duplex.

In some embodiments, a linking group may be conjugated synthetically to the 5′ or 3′ end of the sense strand of an RNAi agent described herein. In some embodiments, a linking group is conjugated synthetically to the 5′ end of the sense strand of an RNAi agent. In some embodiments, a linking group conjugated to an RNAi agent may be a trialkyne linking group.

The following notations are used herein to indicate modified nucleotides, targeting groups, or linking groups:
A=adenosine-3′-phosphate
C=cytidine-3′-phosphate
G=guanosine-3′-phosphate
U=uridine-3′-phosphate
I=inosine-3′-phosphate
a=2′-O-methyladenosine-3′-phosphate
as=2′-O-methyladenosine-3′-phosphorothioate
c=2′-O-methylcytidine-3′-phosphate
cs=2′-O-methylcytidine-3′-phosphorothioate
g=2′-O-methylguanosine-3′-phosphate
gs=2′-O-methylguanosine-3′-phosphorothioate
I=2′-O-methylinosine-3′-phosphate
is=2′-O-methylinosine-3′-phosphorothioate
t=2′-O-methyl-5-methyluridine-3′-phosphate
ts=2′-O-methyl-5-methyluridine-3′-phosphorothioate
u=2′-O-methyluridine-3′-phosphate
us=2′-O-methyluridine-3′-phosphorothioate
Af=2′-fluoroadenosine-3′-phosphate
Afs=2′-fluoroadenosine-3′-phosporothioate
Cf=2′-fluorocytidine-3′-phosphate
Cfs=2′-fluorocytidine-3′-phosphorothioate
Gf=2′-fluoroguanosine-3′-phosphate
Gfs=2′-fluoroguanosine-3′-phosphorothioate
Tf=2′-fluoro-5′-methyluridine-3′-phosphate
Tfs=2′-fluoro-5′-methyluridine-3′-phosphorothioate
Uf=2′-fluorouridine-3′-phosphate
Ufs=2′-fluorouridine-3′-phosphorothioate
dT=2′-deoxythymidine-3′-phosphate
AUNA=2′,3′-seco-adenosine-3′-phosphate
AUNAs=2′,3′-seco-adenosine-3′-phosphorothioate
CUNA=2′,3′-seco-cytidine-3′-phosphate
CUNAs=2′,3′-seco-cytidine-3′-phosphorothioate
GUNA=2′,3′-seco-guanosine-3′-phosphate
GUNAs=2′,3′-seco-guanosine-3′-phosphorothioate
UUNA=2′,3′-seco-uridine-3′-phosphate
UUNAs=2′,3′-seco-uridine-3′-phosphorothioate
s=phosphorothioate linkage
p=terminal phosphate (as synthesized)

Structures of certain modified nucleotides, capping residues and linking groups used herein, are provided in Table 4.

TABLE 4
Structures Representing Various Modified Nucleotides, Capping
Residues, and Linking Groups.

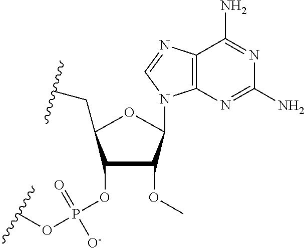

a_2N

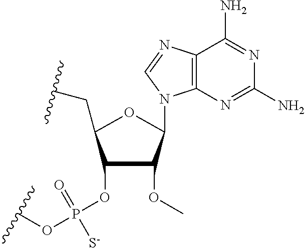

a_2Ns

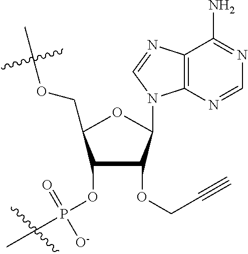

aAlk

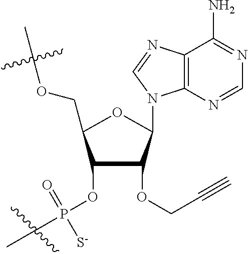

aAlks

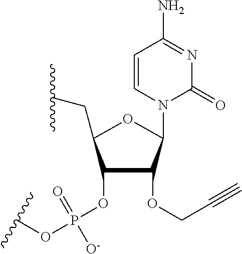

cAlk

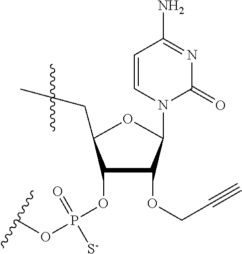

cAlks

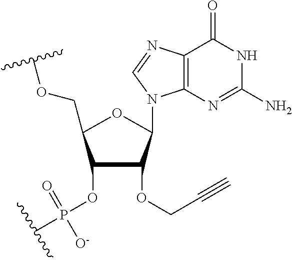

gAlk

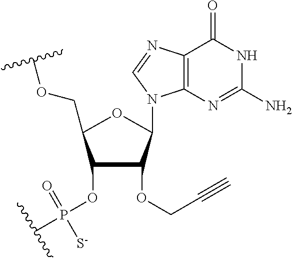

gAlks

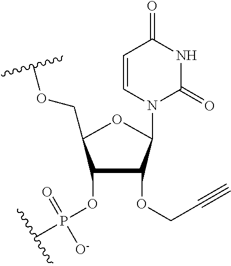

uAlk

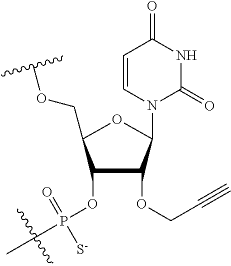

aAlks

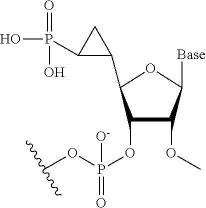

cPrp
When positioned internally:

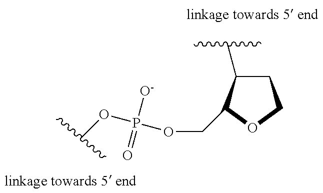

(invAb)
When positioned internally:

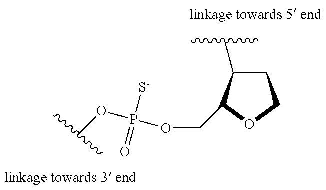

(invAb)s
When positioned at the 3′ terminal end:
Linkage towards 5′ end

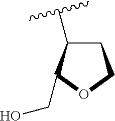

(invAb)
When positioned at the 3′ terminal end of oligonucleotide:

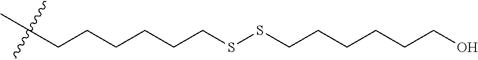 [SMILES: *CCCCCCSSCCCCCCO]

(C6-SS-C6)
When positioned internally in oligonucleotide:

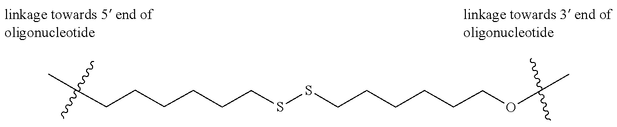 [SMILES: *CCCCCCSSCCCCCCO*]

(C6-SS-C6)

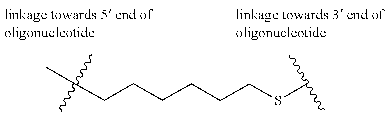 [SMILES: *CCCCCCS*]

(C6-S)
When positioned at the 3′ terminal end of oligonucleotide:

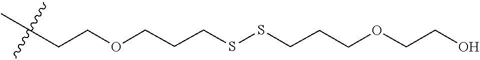 [SMILES: *CCOCCCSSCCCOCCO]

(6-SS-6)
When positioned internally in oligonucleotide:

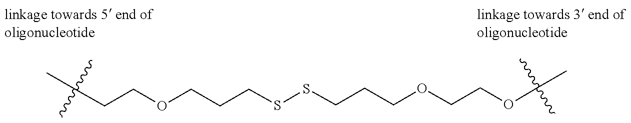 [SMILES: *CCOCCCSSCCCOCCO*]

(6-SS-6)

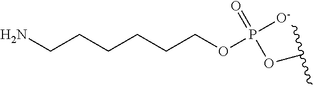 [SMILES: *OP(=O)([O-])OCCCCCCN]

(NH2-C6)

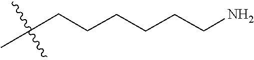 [SMILES: *CCCCCCN]

(C6-NH2)

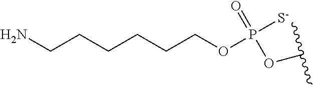 [SMILES: *OP(=O)([S-])OCCCCCCN]

(NH2-C6)s

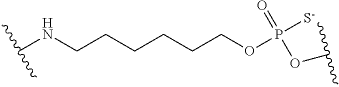 [SMILES: *NCCCCCCOP(=O)([S-])O*]

(NH-C6)s

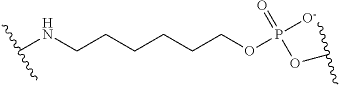 [SMILES: *NCCCCCCOP(=O)([O-])O*]

(NH-C6)

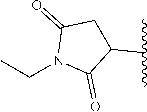 [SMILES: *C1CC(=O)N(CC)C1=O]

nEm or NEM (N-ethylmaleimide)

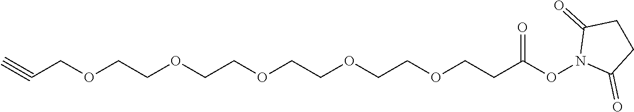 [SMILES: C#CCOCCOCCOCCOCCOCCC(=O)ON1C(=O)CCC1=O]

L6 reactant (BroadPharm ® BP-20907)

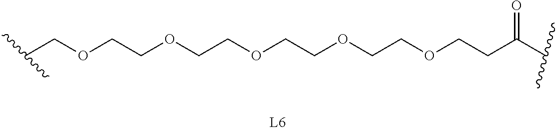 [SMILES: *COCCOCCOCCOCCOCCC(*)=O]

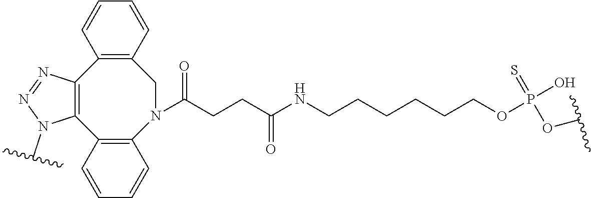 [SMILES: *OP(O)(=S)OCCCCCCNC(=O)CCC(=O)N1Cc2ccccc2-c2nnn(*)c2-c2ccccc21]

DBCO-C6s

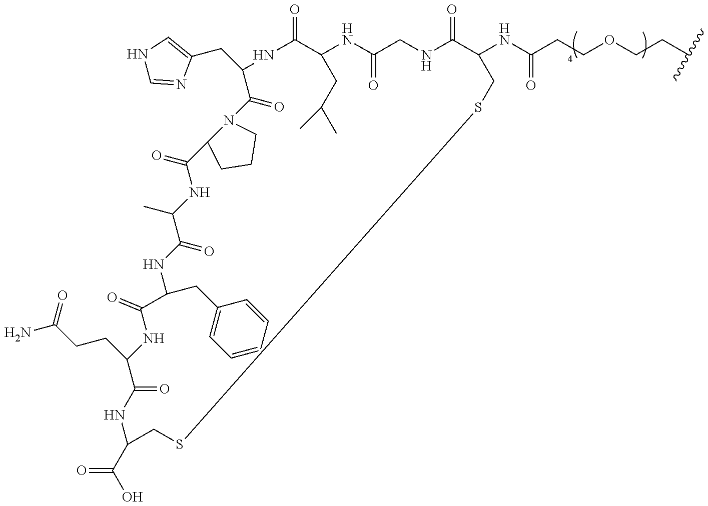

TDA Pep 1 (SEQ ID NO: 77)

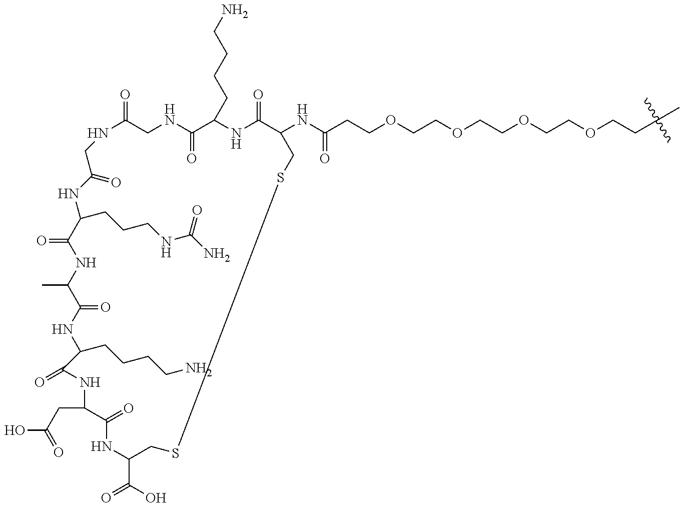 [SMILES: *CCOCCOCCOCCOCCC(=O)NC1CSSCC(C(=O)O)NC(=O)C(CC(=O)O)NC(=O)C(CCCCN)NC(=O)C(C)NC(=O)C(CCCNC(N)=O)NC(=O)CNC(=O)CNC(=O)C(CCCCN)NC1=O]

WAT Homing Pep (SEQ ID NO: 78)

Alternatively, other linking groups known in the art may be used.

In addition or alternatively to linking an RNAi agent to one or more targeting ligands, targeting groups, and/or PK/PD modulators, in some embodiments, a delivery agent may be used to deliver an RNAi agent to a cell or tissue. A delivery agent is a compound that can improve delivery of the RNAi agent to a cell or tissue, and can include, or consist of, but is not limited to: a polymer, such as an amphipathic polymer, a membrane active polymer, a peptide, a melittin peptide, a melittin-like peptide (MLP), a lipid, a reversibly modified polymer or peptide, or a reversibly modified membrane active polyamine.

In some embodiments, the RNAi agents can be combined with lipids, nanoparticles, polymers, liposomes, micelles, DPCs or other delivery systems available in the art. The RNAi agents can also be chemically conjugated to targeting groups, lipids (including, but not limited to cholesterol and cholesteryl derivatives), nanoparticles, polymers, liposomes, micelles, DPCs (see, for example WO 2000/053722, WO 2008/022309, WO 2011/104169, and WO 2012/083185, WO 2013/032829, WO 2013/158141, each of which is incorporated herein by reference), or other delivery systems available in the art.

### Pharmaceutical Compositions

In some embodiments, the present disclosure provides pharmaceutical compositions that include, consist of, or consist essentially of, one or more compounds of Formula (I), LP-4-a, LP-4-b, LP-18-a, LP-18-b, LP-128-a, LP-128-b, LP-151-a, LP-151-b, LP-183-a, LP-183-b, LP-200-a, LP-200-b, LP-208-a, LP-208-b, LP-211-a, LP-211-b, LP-232-a, LP-232-b, LP-242-a, LP-242-b, LP-243-a, LP-243-b, LP-244-a, LP-244-b, LP-245-a, LP-245-b, LP-249-a, LP-249-b, LP-274-a, LP-274-b, LP-295-a, LP-295-b, LP-310-a, LP-310-b, LP-359-a, LP-359-b, LP-361-a, LP-361-b, LP-371-a, LP-371-b, LP-374-a, LP-374-b, LP-375-a, LP-375-b, LP-377-a, LP-377-b, LP-378-a, LP-378-b, LP-379-a, LP-379-b, LP-380-a, LP-380-b, LP-403-a, LP-403-b, LP-404-a, LP-404-b, LP-412-a, LP-412-b, LP-413-a, LP-413-b, LP-416-a, LP-416-b, LP-424-a, LP-424-b, LP-425-a, LP-425-b, LP-426-a, LP-426-b, LP-427-a, LP-427-b, LP-428-a, LP-428-b, LP-432-a, LP-432-b, LP-433-a, LP-433-b, LP-444-a, LP-444-b, LP-445-a, LP-445-b, LP-446-a, LP-446-b, LP-447-a, LP-447-b, LP-453-a, LP-453-b, LP-455-a, LP-455-b, LP-457-a, LP-457-b, LP-458-a, LP-458-b, LP-459-a, LP-459-b, LP-460-a, LP-460-b, LP-461-a, LP-461-b, LP-468-a, LP-468-b, LP-469-a, LP-469-b, LP-470-a, LP-470-b, LP-473-a, LP-473-b, LP-474-a, LP-474-b, CNR1 SM2-a, and/or CNR1 SM2-b.

As used herein, a “pharmaceutical composition” comprises a pharmacologically effective amount of an Active Pharmaceutical Ingredient (API), and optionally one or more pharmaceutically acceptable excipients. Pharmaceutically acceptable excipients (excipients) are substances other than the Active Pharmaceutical ingredient (API, therapeutic product) that are intentionally included in the drug delivery system. Excipients do not exert or are not intended to exert a therapeutic effect at the intended dosage. Excipients may act to a) aid in processing of the drug delivery system during manufacture, b) protect, support or enhance stability, bioavailability or patient acceptability of the API, c) assist in product identification, and/or d) enhance any other attribute of the overall safety, effectiveness, of delivery of the API during storage or use. A pharmaceutically acceptable excipient may or may not be an inert substance.

Excipients include, but are not limited to: absorption enhancers, anti-adherents, anti-foaming agents, anti-oxidants, binders, buffering agents, carriers, coating agents, colors, delivery enhancers, delivery polymers, dextran, dextrose, diluents, disintegrants, emulsifiers, extenders, fillers, flavors, glidants, humectants, lubricants, oils, polymers, preservatives, saline, salts, solvents, sugars, suspending agents, sustained release matrices, sweeteners, thickening agents, tonicity agents, vehicles, water-repelling agents, and wetting agents.

The pharmaceutical compositions described herein can contain other additional components commonly found in pharmaceutical compositions. In some embodiments, the additional component is a pharmaceutically-active material. Pharmaceutically-active materials include, but are not limited to: anti-pruritics, astringents, local anesthetics, or anti-inflammatory agents (e.g., antihistamine, diphenhydramine, etc.), small molecule drug, antibody, antibody fragment, aptamers, and/or vaccines.

The pharmaceutical compositions may also contain preserving agents, solubilizing agents, stabilizing agents, wetting agents, emulsifiers, sweeteners, colorants, odorants, salts for the variation of osmotic pressure, buffers, coating agents, or antioxidants. They may also contain other agent with a known therapeutic benefit.

The pharmaceutical compositions can be administered in a number of ways depending upon whether local or systemic treatment is desired and upon the area to be treated. Administration can be made by any way commonly known in the art, such as, but not limited to, topical (e.g., by a transdermal patch), pulmonary (e.g., by inhalation or insufflation of powders or aerosols, including by nebulizer, intratracheal, intranasal), epidermal, transdermal, oral or parenteral. Parenteral administration includes, but is not limited to, intravenous, intraarterial, subcutaneous, intraperitoneal or intramuscular injection or infusion; subdermal (e.g., via an implanted device), intracranial, intraparenchymal, intrathecal, and intraventricular, administration. In some embodiments, the pharmaceutical compositions described herein are administered by subcutaneous injection. The pharmaceutical compositions may be administered orally, for example in the form of tablets, coated tablets, dragées, hard or soft gelatin capsules, solutions, emulsions or suspensions. Administration can also be carried out rectally, for example using suppositories; locally or percutaneously, for example using ointments, creams, gels, or solutions; or parenterally, for example using injectable solutions.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor® EL (BASF, Parsippany, NJ) or phosphate buffered saline. It should be stable under the conditions of manufacture and storage and should be preserved against the contaminating action of microorganisms such as bacteria and fungi. The carrier can be a solvent or dispersion medium containing, for example, water, ethanol, polyol (for example, glycerol, propylene glycol, and liquid polyethylene glycol), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filter sterilization. Generally, dispersions are prepared by incorporating the active compound into a sterile vehicle which contains a basic dispersion medium and the required other ingredients from those enumerated above. In the case of sterile powders for the preparation of sterile injectable solutions, methods of preparation include vacuum drying and freeze-drying which yields a powder of the active ingredient plus any additional desired ingredient from a previously sterile-filtered solution thereof.

Formulations suitable for intra-articular administration can be in the form of a sterile aqueous preparation of any of the ligands described herein that can be in microcrystalline form, for example, in the form of an aqueous microcrystalline suspension. Liposomal formulations or biodegradable polymer systems can also be used to present any of the ligands described herein for both intra-articular and ophthalmic administration.

The active compounds can be prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions can also be used as pharmaceutically acceptable carriers. These can be prepared according to methods known to those skilled in the art, for example, as described in U.S. Pat. No. 4,522,811.

A pharmaceutical composition can contain other additional components commonly found in pharmaceutical compositions. Such additional components include, but are not limited to: anti-pruritics, astringents, local anesthetics, or anti-inflammatory agents (e.g., antihistamine, diphenhydramine, etc.). As used herein, “pharmacologically effective amount,” “therapeutically effective amount,” or simply “effective amount” refers to that amount of a pharmaceutically active agent to produce a pharmacological, therapeutic, or preventive result.

Medicaments containing compounds of Formula LP-4-a, LP-4-b, LP-18-a, LP-18-b, LP-128-a, LP-128-b, LP-151-a, LP-151-b, LP-183-a, LP-183-b, LP-200-a, LP-200-b, LP-208-a, LP-208-b, LP-211-a, LP-211-b, LP-232-a, LP-232-b, LP-242-a, LP-242-b, LP-243-a, LP-243-b, LP-244-a, LP-244-b, LP-245-a, LP-245-b, LP-249-a, LP-249-b, LP-274-a, LP-274-b, LP-295-a, LP-295-b, LP-310-a, LP-310-b, LP-359-a, LP-359-b, LP-361-a, LP-361-b, LP-371-a, LP-371-b, LP-374-a, LP-374-b, LP-375-a, LP-375-b, LP-377-a, LP-377-b, LP-378-a, LP-378-b, LP-379-a, LP-379-b, LP-380-a, LP-380-b, LP-403-a, LP-403-b, LP-404-a, LP-404-b, LP-412-a, LP-412-b, LP-413-a, LP-413-b, LP-416-a, LP-416-b, LP-424-a, LP-424-b, LP-425-a, LP-425-b, LP-426-a, LP-426-b, LP-427-a, LP-427-b, LP-428-a, LP-428-b, LP-432-a, LP-432-b, LP-433-a, LP-433-b, LP-444-a, LP-444-b, LP-445-a, LP-445-b, LP-446-a, LP-446-b, LP-447-a, LP-447-b, LP-453-a, LP-453-b, LP-455-a, LP-455-b, LP-457-a, LP-457-b, LP-458-a, LP-458-b, LP-459-a, LP-459-b, LP-460-a, LP-460-b, LP-461-a, LP-461-b, LP-468-a, LP-468-b, LP-469-a, LP-469-b, LP-470-a, LP-470-b, LP-473-a, LP-473-b, LP-474-a, LP-474-b, CNR1 SM2-a, or CNR1 SM2-b, are also an object of the present invention, as are processes for the manufacture of such medicaments, which processes comprise bringing one or more compound of Formula LP-4-a, LP-4-b, LP-18-a, LP-18-b, LP-128-a, LP-128-b, LP-151-a, LP-151-b, LP-183-a, LP-183-b, LP-200-a, LP-200-b, LP-208-a, LP-208-b, LP-211-a, LP-211-b, LP-232-a, LP-232-b, LP-242-a, LP-242-b, LP-243-a, LP-243-b, LP-244-a, LP-244-b, LP-245-a, LP-245-b, LP-249-a, LP-249-b, LP-274-a, LP-274-b, LP-295-a, LP-295-b, LP-310-a, LP-310-b, LP-359-a, LP-359-b, LP-361-a, LP-361-b, LP-371-a, LP-371-b, LP-374-a, LP-374-b, LP-375-a, LP-375-b, LP-377-a, LP-377-b, LP-378-a, LP-378-b, LP-379-a, LP-379-b, LP-380-a, LP-380-b, LP-403-a, LP-403-b, LP-404-a, LP-404-b, LP-412-a, LP-412-b, LP-413-a, LP-413-b, LP-416-a, LP-416-b, LP-424-a, LP-424-b, LP-425-a, LP-425-b, LP-426-a, LP-426-b, LP-427-a, LP-427-b, LP-428-a, LP-428-b, LP-432-a, LP-432-b, LP-433-a, LP-433-b, LP-444-a, LP-444-b, LP-445-a, LP-445-b, LP-446-a, LP-446-b, LP-447-a, LP-447-b, LP-453-a, LP-453-b, LP-455-a, LP-455-b, LP-457-a, LP-457-b, LP-458-a, LP-458-b, LP-459-a, LP-459-b, LP-460-a, LP-460-b, LP-461-a, LP-461-b, LP-468-a, LP-468-b, LP-469-a, LP-469-b, LP-470-a, LP-470-b, LP-473-a, LP-473-b, LP-474-a, LP-474-b, CNR1 SM2-a, or CNR1 SM2-b, and, if desired, one or more other substances with a known therapeutic benefit, into a pharmaceutically acceptable form.

The described compounds of Formula LP-4-a, LP-4-b, LP-18-a, LP-18-b, LP-128-a, LP-128-b, LP-151-a, LP-151-b, LP-183-a, LP-183-b, LP-200-a, LP-200-b, LP-208-a, LP-208-b, LP-211-a, LP-211-b, LP-232-a, LP-232-b, LP-242-a, LP-242-b, LP-243-a, LP-243-b, LP-244-a, LP-244-b, LP-245-a, LP-245-b, LP-249-a, LP-249-b, LP-274-a, LP-274-b, LP-295-a, LP-295-b, LP-310-a, LP-310-b, LP-359-a, LP-359-b, LP-361-a, LP-361-b, LP-371-a, LP-371-b, LP-374-a, LP-374-b, LP-375-a, LP-375-b, LP-377-a, LP-377-b, LP-378-a, LP-378-b, LP-379-a, LP-379-b, LP-380-a, LP-380-b, LP-403-a, LP-403-b, LP-404-a, LP-404-b, LP-412-a, LP-412-b, LP-413-a, LP-413-b, LP-416-a, LP-416-b, LP-424-a, LP-424-b, LP-425-a, LP-425-b, LP-426-a, LP-426-b, LP-427-a, LP-427-b, LP-428-a, LP-428-b, LP-432-a, LP-432-b, LP-433-a, LP-433-b, LP-444-a, LP-444-b, LP-445-a, LP-445-b, LP-446-a, LP-446-b, LP-447-a, LP-447-b, LP-453-a, LP-453-b, LP-455-a, LP-455-b, LP-457-a, LP-457-b, LP-458-a, LP-458-b, LP-459-a, LP-459-b, LP-460-a, LP-460-b, LP-461-a, LP-461-b, LP-468-a, LP-468-b, LP-469-a, LP-469-b, LP-470-a, LP-470-b, LP-473-a, LP-473-b, LP-474-a, LP-474-b, CNR1 SM2-a, or CNR1 SM2-b, and pharmaceutical compositions comprising compounds of Formula LP-4-a, LP-4-b, LP-18-a, LP-18-b, LP-128-a, LP-128-b, LP-151-a, LP-151-b, LP-183-a, LP-183-b, LP-200-a, LP-200-b, LP-208-a, LP-208-b, LP-211-a, LP-211-b, LP-232-a, LP-232-b, LP-242-a, LP-242-b, LP-243-a, LP-243-b, LP-244-a, LP-244-b, LP-245-a, LP-245-b, LP-249-a, LP-249-b, LP-274-a, LP-274-b, LP-295-a, LP-295-b, LP-310-a, LP-310-b, LP-359-a, LP-359-b, LP-361-a, LP-361-b, LP-371-a, LP-371-b, LP-374-a, LP-374-b, LP-375-a, LP-375-b, LP-377-a, LP-377-b, LP-378-a, LP-378-b, LP-379-a, LP-379-b, LP-380-a, LP-380-b, LP-403-a, LP-403-b, LP-404-a, LP-404-b, LP-412-a, LP-412-b, LP-413-a, LP-413-b, LP-416-a, LP-416-b, LP-424-a, LP-424-b, LP-425-a, LP-425-b, LP-426-a, LP-426-b, LP-427-a, LP-427-b, LP-428-a, LP-428-b, LP-432-a, LP-432-b, LP-433-a, LP-433-b, LP-444-a, LP-444-b, LP-445-a, LP-445-b, LP-446-a, LP-446-b, LP-447-a, LP-447-b, LP-453-a, LP-453-b, LP-455-a, LP-455-b, LP-457-a, LP-457-b, LP-458-a, LP-458-b, LP-459-a, LP-459-b, LP-460-a, LP-460-b, LP-461-a, LP-461-b, LP-468-a, LP-468-b, LP-469-a, LP-469-b, LP-470-a, LP-470-b, LP-473-a, LP-473-b, LP-474-a, LP-474-b, CNR1 SM2-a, or CNR1 SM2-b, disclosed herein may be packaged or included in a kit, container, pack, or dispenser. The compounds of Formula LP-4-a, LP-4-b, LP-18-a, LP-18-b, LP-128-a, LP-128-b, LP-151-a, LP-151-b, LP-183-a, LP-183-b, LP-200-a, LP-200-b, LP-208-a, LP-208-b, LP-211-a, LP-211-b, LP-232-a, LP-232-b, LP-242-a, LP-242-b, LP-243-a, LP-243-b, LP-244-a, LP-244-b, LP-245-a, LP-245-b, LP-249-a, LP-249-b, LP-274-a, LP-274-b, LP-295-a, LP-295-b, LP-310-a, LP-310-b, LP-359-a, LP-359-b, LP-361-a, LP-361-b, LP-371-a, LP-371-b, LP-374-a, LP-374-b, LP-375-a, LP-375-b, LP-377-a, LP-377-b, LP-378-a, LP-378-b, LP-379-a, LP-379-b, LP-380-a, LP-380-b, LP-403-a, LP-403-b, LP-404-a, LP-404-b, LP-412-a, LP-412-b, LP-413-a, LP-413-b, LP-416-a, LP-416-b, LP-424-a, LP-424-b, LP-425-a, LP-425-b, LP-426-a, LP-426-b, LP-427-a, LP-427-b, LP-428-a, LP-428-b, LP-432-a, LP-432-b, LP-433-a, LP-433-b, LP-444-a, LP-444-b, LP-445-a, LP-445-b, LP-446-a, LP-446-b, LP-447-a, LP-447-b, LP-453-a, LP-453-b, LP-455-a, LP-455-b, LP-457-a, LP-457-b, LP-458-a, LP-458-b, LP-459-a, LP-459-b, LP-460-a, LP-460-b, LP-461-a, LP-461-b, LP-468-a, LP-468-b, LP-469-a, LP-469-b, LP-470-a, LP-470-b, LP-473-a, LP-473-b, LP-474-a, LP-474-b, CNR1 SM2-a, or CNR1 SM2-b, and pharmaceutical compositions comprising the compounds of Formula LP-4-a, LP-4-b, LP-18-a, LP-18-b, LP-128-a, LP-128-b, LP-151-a, LP-151-b, LP-183-a, LP-183-b, LP-200-a, LP-200-b, LP-208-a, LP-208-b, LP-211-a, LP-211-b, LP-232-a, LP-232-b, LP-242-a, LP-242-b, LP-243-a, LP-243-b, LP-244-a, LP-244-b, LP-245-a, LP-245-b, LP-249-a, LP-249-b, LP-274-a, LP-274-b, LP-295-a, LP-295-b, LP-310-a, LP-310-b, LP-359-a, LP-359-b, LP-361-a, LP-361-b, LP-371-a, LP-371-b, LP-374-a, LP-374-b, LP-375-a, LP-375-b, LP-377-a, LP-377-b, LP-378-a, LP-378-b, LP-379-a, LP-379-b, LP-380-a, LP-380-b, LP-403-a, LP-403-b, LP-404-a, LP-404-b, LP-412-a, LP-412-b, LP-413-a, LP-413-b, LP-416-a, LP-416-b, LP-424-a, LP-424-b, LP-425-a, LP-425-b, LP-426-a, LP-426-b, LP-427-a, LP-427-b, LP-428-a, LP-428-b, LP-432-a, LP-432-b, LP-433-a, LP-433-b, LP-444-a, LP-444-b, LP-445-a, LP-445-b, LP-446-a, LP-446-b, LP-447-a, LP-447-b, LP-453-a, LP-453-b, LP-455-a, LP-455-b, LP-457-a, LP-457-b, LP-458-a, LP-458-b, LP-459-a, LP-459-b, LP-460-a, LP-460-b, LP-461-a, LP-461-b, LP-468-a, LP-468-b, LP-469-a, LP-469-b, LP-470-a, LP-470-b, LP-473-a, LP-473-b, LP-474-a, LP-474-b, CNR1 SM2-a, or CNR1 SM2-b, may be packaged in pre-filled syringes or vials.

### Methods of Treatment and Inhibition of Expression

The compounds of Formula (I), LP-4-a, LP-4-b, LP-18-a, LP-18-b, LP-128-a, LP-128-b, LP-151-a, LP-151-b, LP-183-a, LP-183-b, LP-200-a, LP-200-b, LP-208-a, LP-208-b, LP-211-a, LP-211-b, LP-232-a, LP-232-b, LP-242-a, LP-242-b, LP-243-a, LP-243-b, LP-244-a, LP-244-b, LP-245-a, LP-245-b, LP-249-a, LP-249-b, LP-274-a, LP-274-b, LP-295-a, LP-295-b, LP-310-a, LP-310-b, LP-359-a, LP-359-b, LP-361-a, LP-361-b, LP-371-a, LP-371-b, LP-374-a, LP-374-b, LP-375-a, LP-375-b, LP-377-a, LP-377-b, LP-378-a, LP-378-b, LP-379-a, LP-379-b, LP-380-a, LP-380-b, LP-403-a, LP-403-b, LP-404-a, LP-404-b, LP-412-a, LP-412-b, LP-413-a, LP-413-b, LP-416-a, LP-416-b, LP-424-a, LP-424-b, LP-425-a, LP-425-b, LP-426-a, LP-426-b, LP-427-a, LP-427-b, LP-428-a, LP-428-b, LP-432-a, LP-432-b, LP-433-a, LP-433-b, LP-444-a, LP-444-b, LP-445-a, LP-445-b, LP-446-a, LP-446-b, LP-447-a, LP-447-b, LP-453-a, LP-453-b, LP-455-a, LP-455-b, LP-457-a, LP-457-b, LP-458-a, LP-458-b, LP-459-a, LP-459-b, LP-460-a, LP-460-b, LP-461-a, LP-461-b, LP-468-a, LP-468-b, LP-469-a, LP-469-b, LP-470-a, LP-470-b, LP-473-a, LP-473-b, LP-474-a, LP-474-b, CNR1 SM2-a, or CNR1 SM2-b, disclosed herein can be used to treat a subject (e.g., a human or other mammal) having a disease or disorder that would benefit from administration of such compounds. In some embodiments, the compounds of Formula (I), LP-4-a, LP-4-b, LP-18-a, LP-18-b, LP-128-a, LP-128-b, LP-151-a, LP-151-b, LP-183-a, LP-183-b, LP-200-a, LP-200-b, LP-208-a, LP-208-b, LP-211-a, LP-211-b, LP-232-a, LP-232-b, LP-242-a, LP-242-b, LP-243-a, LP-243-b, LP-244-a, LP-244-b, LP-245-a, LP-245-b, LP-249-a, LP-249-b, LP-274-a, LP-274-b, LP-295-a, LP-295-b, LP-310-a, LP-310-b, LP-359-a, LP-359-b, LP-361-a, LP-361-b, LP-371-a, LP-371-b, LP-374-a, LP-374-b, LP-375-a, LP-375-b, LP-377-a, LP-377-b, LP-378-a, LP-378-b, LP-379-a, LP-379-b, LP-380-a, LP-380-b, LP-403-a, LP-403-b, LP-404-a, LP-404-b, LP-412-a, LP-412-b, LP-413-a, LP-413-b, LP-416-a, LP-416-b, LP-424-a, LP-424-b, LP-425-a, LP-425-b, LP-426-a, LP-426-b, LP-427-a, LP-427-b, LP-428-a, LP-428-b, LP-432-a, LP-432-b, LP-433-a, LP-433-b, LP-444-a, LP-444-b, LP-445-a, LP-445-b, LP-446-a, LP-446-b, LP-447-a, LP-447-b, LP-453-a, LP-453-b, LP-455-a, LP-455-b, LP-457-a, LP-457-b, LP-458-a, LP-458-b, LP-459-a, LP-459-b, LP-460-a, LP-460-b, LP-461-a, LP-461-b, LP-468-a, LP-468-b, LP-469-a, LP-469-b, LP-470-a, LP-470-b, LP-473-a, LP-473-b, LP-474-a, LP-474-b, CNR1 SM2-a, or CNR1 SM2-b, disclosed herein can be used to treat a subject (e.g., a human) that would benefit from reduction and/or inhibition in expression of a target mRNA and/or protein levels, for example, a subject that has been diagnosed with or is suffering from symptoms related to a adipose disease or disorder.

The present disclosure also provides methods of modulating (i.e., inhibiting or increasing) the activity (e.g., aberrant activity, such as increased or decreased activity) of a gene in the adipose tissue of a subject, biological sample, or cell (e.g., an adipocyte) comprising administering an effective amount of one or more compounds of Formula (I) to the subject, biological sample, or cell. The present disclosure also provides methods for the treatment of a wide range of diseases, such as diseases (e.g., metabolic diseases) associated with aberrant activity (e.g., increased or decreased activity) of a gene expressed in adipose tissue or in an adipocyte in a subject in need thereof comprising administering a therapeutically effective amount of one or more compounds of Formula (I) to the subject.

In some embodiments, the subject is administered a therapeutically effective amount of one or more compounds of Formula (I), LP-4-a, LP-4-b, LP-18-a, LP-18-b, LP-128-a, LP-128-b, LP-151-a, LP-151-b, LP-183-a, LP-183-b, LP-200-a, LP-200-b, LP-208-a, LP-208-b, LP-211-a, LP-211-b, LP-232-a, LP-232-b, LP-242-a, LP-242-b, LP-243-a, LP-243-b, LP-244-a, LP-244-b, LP-245-a, LP-245-b, LP-249-a, LP-249-b, LP-274-a, LP-274-b, LP-295-a, LP-295-b, LP-310-a, LP-310-b, LP-359-a, LP-359-b, LP-361-a, LP-361-b, LP-371-a, LP-371-b, LP-374-a, LP-374-b, LP-375-a, LP-375-b, LP-377-a, LP-377-b, LP-378-a, LP-378-b, LP-379-a, LP-379-b, LP-380-a, LP-380-b, LP-403-a, LP-403-b, LP-404-a, LP-404-b, LP-412-a, LP-412-b, LP-413-a, LP-413-b, LP-416-a, LP-416-b, LP-424-a, LP-424-b, LP-425-a, LP-425-b, LP-426-a, LP-426-b, LP-427-a, LP-427-b, LP-428-a, LP-428-b, LP-432-a, LP-432-b, LP-433-a, LP-433-b, LP-444-a, LP-444-b, LP-445-a, LP-445-b, LP-446-a, LP-446-b, LP-447-a, LP-447-b, LP-453-a, LP-453-b, LP-455-a, LP-455-b, LP-457-a, LP-457-b, LP-458-a, LP-458-b, LP-459-a, LP-459-b, LP-460-a, LP-460-b, LP-461-a, LP-461-b, LP-468-a, LP-468-b, LP-469-a, LP-469-b, LP-470-a, LP-470-b, LP-473-a, LP-473-b, LP-474-a, LP-474-b, CNR1 SM2-a, or CNR1 SM2-b, disclosed herein. Treatment of a subject can include therapeutic and/or prophylactic treatment. The subject is administered a therapeutically effective amount of one or more compounds of Formula LP-4-a, LP-4-b, LP-18-a, LP-18-b, LP-128-a, LP-128-b, LP-151-a, LP-151-b, LP-183-a, LP-183-b, LP-200-a, LP-200-b, LP-208-a, LP-208-b, LP-211-a, LP-211-b, LP-232-a, LP-232-b, LP-242-a, LP-242-b, LP-243-a, LP-243-b, LP-244-a, LP-244-b, LP-245-a, LP-245-b, LP-249-a, LP-249-b, LP-274-a, LP-274-b, LP-295-a, LP-295-b, LP-310-a, LP-310-b, LP-359-a, LP-359-b, LP-361-a, LP-361-b, LP-371-a, LP-371-b, LP-374-a, LP-374-b, LP-375-a, LP-375-b, LP-377-a, LP-377-b, LP-378-a, LP-378-b, LP-379-a, LP-379-b, LP-380-a, LP-380-b, LP-403-a, LP-403-b, LP-404-a, LP-404-b, LP-412-a, LP-412-b, LP-413-a, LP-413-b, LP-416-a, LP-416-b, LP-424-a, LP-424-b, LP-425-a, LP-425-b, LP-426-a, LP-426-b, LP-427-a, LP-427-b, LP-428-a, LP-428-b, LP-432-a, LP-432-b, LP-433-a, LP-433-b, LP-444-a, LP-444-b, LP-445-a, LP-445-b, LP-446-a, LP-446-b, LP-447-a, LP-447-b, LP-453-a, LP-453-b, LP-455-a, LP-455-b, LP-457-a, LP-457-b, LP-458-a, LP-458-b, LP-459-a, LP-459-b, LP-460-a, LP-460-b, LP-461-a, LP-461-b, LP-468-a, LP-468-b, LP-469-a, LP-469-b, LP-470-a, LP-470-b, LP-473-a, LP-473-b, LP-474-a, LP-474-b, CNR1 SM2-a, or CNR1 SM2-b, described herein. The subject can be a human, patient, or human patient. The subject may be an adult, adolescent, child, or infant. Administration of a pharmaceutical composition described herein can be to a human being or animal.

The compounds of Formula LP-4-a, LP-4-b, LP-18-a, LP-18-b, LP-128-a, LP-128-b, LP-151-a, LP-151-b, LP-183-a, LP-183-b, LP-200-a, LP-200-b, LP-208-a, LP-208-b, LP-211-a, LP-211-b, LP-232-a, LP-232-b, LP-242-a, LP-242-b, LP-243-a, LP-243-b, LP-244-a, LP-244-b, LP-245-a, LP-245-b, LP-249-a, LP-249-b, LP-274-a, LP-274-b, LP-295-a, LP-295-b, LP-310-a, LP-310-b, LP-359-a, LP-359-b, LP-361-a, LP-361-b, LP-371-a, LP-371-b, LP-374-a, LP-374-b, LP-375-a, LP-375-b, LP-377-a, LP-377-b, LP-378-a, LP-378-b, LP-379-a, LP-379-b, LP-380-a, LP-380-b, LP-403-a, LP-403-b, LP-404-a, LP-404-b, LP-412-a, LP-412-b, LP-413-a, LP-413-b, LP-416-a, LP-416-b, LP-424-a, LP-424-b, LP-425-a, LP-425-b, LP-426-a, LP-426-b, LP-427-a, LP-427-b, LP-428-a, LP-428-b, LP-432-a, LP-432-b, LP-433-a, LP-433-b, LP-444-a, LP-444-b, LP-445-a, LP-445-b, LP-446-a, LP-446-b, LP-447-a, LP-447-b, LP-453-a, LP-453-b, LP-455-a, LP-455-b, LP-457-a, LP-457-b, LP-458-a, LP-458-b, LP-459-a, LP-459-b, LP-460-a, LP-460-b, LP-461-a, LP-461-b, LP-468-a, LP-468-b, LP-469-a, LP-469-b, LP-470-a, LP-470-b, LP-473-a, LP-473-b, LP-474-a, LP-474-b, CNR1 SM2-a, or CNR1 SM2-b, described herein can be used to treat at least one symptom in a subject having a disease or disorder related to a target gene (e.g., ALK7, Adipoq), or having a disease or disorder that is mediated at least in part by the expression of the target gene (e.g., ALK7, Adipoq). In some embodiments, the compounds of Formula LP-4-a, LP-4-b, LP-18-a, LP-18-b, LP-128-a, LP-128-b, LP-151-a, LP-151-b, LP-183-a, LP-183-b, LP-200-a, LP-200-b, LP-208-a, LP-208-b, LP-211-a, LP-211-b, LP-232-a, LP-232-b, LP-242-a, LP-242-b, LP-243-a, LP-243-b, LP-244-a, LP-244-b, LP-245-a, LP-245-b, LP-249-a, LP-249-b, LP-274-a, LP-274-b, LP-295-a, LP-295-b, LP-310-a, LP-310-b, LP-359-a, LP-359-b, LP-361-a, LP-361-b, LP-371-a, LP-371-b, LP-374-a, LP-374-b, LP-375-a, LP-375-b, LP-377-a, LP-377-b, LP-378-a, LP-378-b, LP-379-a, LP-379-b, LP-380-a, LP-380-b, LP-403-a, LP-403-b, LP-404-a, LP-404-b, LP-412-a, LP-412-b, LP-413-a, LP-413-b, LP-416-a, LP-416-b, LP-424-a, LP-424-b, LP-425-a, LP-425-b, LP-426-a, LP-426-b, LP-427-a, LP-427-b, LP-428-a, LP-428-b, LP-432-a, LP-432-b, LP-433-a, LP-433-b, LP-444-a, LP-444-b, LP-445-a, LP-445-b, LP-446-a, LP-446-b, LP-447-a, LP-447-b, LP-453-a, LP-453-b, LP-455-a, LP-455-b, LP-457-a, LP-457-b, LP-458-a, LP-458-b, LP-459-a, LP-459-b, LP-460-a, LP-460-b, LP-461-a, LP-461-b, LP-468-a, LP-468-b, LP-469-a, LP-469-b, LP-470-a, LP-470-b, LP-473-a, LP-473-b, LP-474-a, LP-474-b, CNR1 SM2-a, or CNR1 SM2-b, are used to treat or manage a clinical presentation of a subject with a disease or disorder that would benefit from or be mediated at least in party by a reduction in mRNA of a target gene. The subject is administered a therapeutically effective amount of one or more of the compounds of Formula LP-4-a, LP-4-b, LP-18-a, LP-18-b, LP-128-a, LP-128-b, LP-151-a, LP-151-b, LP-183-a, LP-183-b, LP-200-a, LP-200-b, LP-208-a, LP-208-b, LP-211-a, LP-211-b, LP-232-a, LP-232-b, LP-242-a, LP-242-b, LP-243-a, LP-243-b, LP-244-a, LP-244-b, LP-245-a, LP-245-b, LP-249-a, LP-249-b, LP-274-a, LP-274-b, LP-295-a, LP-295-b, LP-310-a, LP-310-b, LP-359-a, LP-359-b, LP-361-a, LP-361-b, LP-371-a, LP-371-b, LP-374-a, LP-374-b, LP-375-a, LP-375-b, LP-377-a, LP-377-b, LP-378-a, LP-378-b, LP-379-a, LP-379-b, LP-380-a, LP-380-b, LP-403-a, LP-403-b, LP-404-a, LP-404-b, LP-412-a, LP-412-b, LP-413-a, LP-413-b, LP-416-a, LP-416-b, LP-424-a, LP-424-b, LP-425-a, LP-425-b, LP-426-a, LP-426-b, LP-427-a, LP-427-b, LP-428-a, LP-428-b, LP-432-a, LP-432-b, LP-433-a, LP-433-b, LP-444-a, LP-444-b, LP-445-a, LP-445-b, LP-446-a, LP-446-b, LP-447-a, LP-447-b, LP-453-a, LP-453-b, LP-455-a, LP-455-b, LP-457-a, LP-457-b, LP-458-a, LP-458-b, LP-459-a, LP-459-b, LP-460-a, LP-460-b, LP-461-a, LP-461-b, LP-468-a, LP-468-b, LP-469-a, LP-469-b, LP-470-a, LP-470-b, LP-473-a, LP-473-b, LP-474-a, LP-474-b, CNR1 SM2-a, or CNR1 SM2-b, or compositions described herein. In some embodiments, the methods disclosed herein comprise administering a composition comprising a compound of Formula LP-4-a, LP-4-b, LP-18-a, LP-18-b, LP-128-a, LP-128-b, LP-151-a, LP-151-b, LP-183-a, LP-183-b, LP-200-a, LP-200-b, LP-208-a, LP-208-b, LP-211-a, LP-211-b, LP-232-a, LP-232-b, LP-242-a, LP-242-b, LP-243-a, LP-243-b, LP-244-a, LP-244-b, LP-245-a, LP-245-b, LP-249-a, LP-249-b, LP-274-a, LP-274-b, LP-295-a, LP-295-b, LP-310-a, LP-310-b, LP-359-a, LP-359-b, LP-361-a, LP-361-b, LP-371-a, LP-371-b, LP-374-a, LP-374-b, LP-375-a, LP-375-b, LP-377-a, LP-377-b, LP-378-a, LP-378-b, LP-379-a, LP-379-b, LP-380-a, LP-380-b, LP-403-a, LP-403-b, LP-404-a, LP-404-b, LP-412-a, LP-412-b, LP-413-a, LP-413-b, LP-416-a, LP-416-b, LP-424-a, LP-424-b, LP-425-a, LP-425-b, LP-426-a, LP-426-b, LP-427-a, LP-427-b, LP-428-a, LP-428-b, LP-432-a, LP-432-b, LP-433-a, LP-433-b, LP-444-a, LP-444-b, LP-445-a, LP-445-b, LP-446-a, LP-446-b, LP-447-a, LP-447-b, LP-453-a, LP-453-b, LP-455-a, LP-455-b, LP-457-a, LP-457-b, LP-458-a, LP-458-b, LP-459-a, LP-459-b, LP-460-a, LP-460-b, LP-461-a, LP-461-b, LP-468-a, LP-468-b, LP-469-a, LP-469-b, LP-470-a, LP-470-b, LP-473-a, LP-473-b, LP-474-a, LP-474-b, CNR1 SM2-a, or CNR1 SM2-b, described herein to a subject to be treated. In some embodiments, the subject is administered a prophylactically effective amount of any one or more of the described compounds of Formula LP-4-a, LP-4-b, LP-18-a, LP-18-b, LP-128-a, LP-128-b, LP-151-a, LP-151-b, LP-183-a, LP-183-b, LP-200-a, LP-200-b, LP-208-a, LP-208-b, LP-211-a, LP-211-b, LP-232-a, LP-232-b, LP-242-a, LP-242-b, LP-243-a, LP-243-b, LP-244-a, LP-244-b, LP-245-a, LP-245-b, LP-249-a, LP-249-b, LP-274-a, LP-274-b, LP-295-a, LP-295-b, LP-310-a, LP-310-b, LP-359-a, LP-359-b, LP-361-a, LP-361-b, LP-371-a, LP-371-b, LP-374-a, LP-374-b, LP-375-a, LP-375-b, LP-377-a, LP-377-b, LP-378-a, LP-378-b, LP-379-a, LP-379-b, LP-380-a, LP-380-b, LP-403-a, LP-403-b, LP-404-a, LP-404-b, LP-412-a, LP-412-b, LP-413-a, LP-413-b, LP-416-a, LP-416-b, LP-424-a, LP-424-b, LP-425-a, LP-425-b, LP-426-a, LP-426-b, LP-427-a, LP-427-b, LP-428-a, LP-428-b, LP-432-a, LP-432-b, LP-433-a, LP-433-b, LP-444-a, LP-444-b, LP-445-a, LP-445-b, LP-446-a, LP-446-b, LP-447-a, LP-447-b, LP-453-a, LP-453-b, LP-455-a, LP-455-b, LP-457-a, LP-457-b, LP-458-a, LP-458-b, LP-459-a, LP-459-b, LP-460-a, LP-460-b, LP-461-a, LP-461-b, LP-468-a, LP-468-b, LP-469-a, LP-469-b, LP-470-a, LP-470-b, LP-473-a, LP-473-b, LP-474-a, LP-474-b, CNR1 SM2-a, or CNR1 SM2-b, thereby treating the subject by preventing or inhibiting the at least one symptom.

In certain embodiments, the present disclosure provides methods for treatment of diseases, disorders, conditions, or pathological states mediated at least in part by target gene expression, in a patient in need thereof, wherein the methods include administering to the patient any of the compounds of Formula LP-4-a, LP-4-b, LP-18-a, LP-18-b, LP-128-a, LP-128-b, LP-151-a, LP-151-b, LP-183-a, LP-183-b, LP-200-a, LP-200-b, LP-208-a, LP-208-b, LP-211-a, LP-211-b, LP-232-a, LP-232-b, LP-242-a, LP-242-b, LP-243-a, LP-243-b, LP-244-a, LP-244-b, LP-245-a, LP-245-b, LP-249-a, LP-249-b, LP-274-a, LP-274-b, LP-295-a, LP-295-b, LP-310-a, LP-310-b, LP-359-a, LP-359-b, LP-361-a, LP-361-b, LP-371-a, LP-371-b, LP-374-a, LP-374-b, LP-375-a, LP-375-b, LP-377-a, LP-377-b, LP-378-a, LP-378-b, LP-379-a, LP-379-b, LP-380-a, LP-380-b, LP-403-a, LP-403-b, LP-404-a, LP-404-b, LP-412-a, LP-412-b, LP-413-a, LP-413-b, LP-416-a, LP-416-b, LP-424-a, LP-424-b, LP-425-a, LP-425-b, LP-426-a, LP-426-b, LP-427-a, LP-427-b, LP-428-a, LP-428-b, LP-432-a, LP-432-b, LP-433-a, LP-433-b, LP-444-a, LP-444-b, LP-445-a, LP-445-b, LP-446-a, LP-446-b, LP-447-a, LP-447-b, LP-453-a, LP-453-b, LP-455-a, LP-455-b, LP-457-a, LP-457-b, LP-458-a, LP-458-b, LP-459-a, LP-459-b, LP-460-a, LP-460-b, LP-461-a, LP-461-b, LP-468-a, LP-468-b, LP-469-a, LP-469-b, LP-470-a, LP-470-b, LP-473-a, LP-473-b, LP-474-a, LP-474-b, CNR1 SM2-a, or CNR1 SM2-b, described herein.

In some embodiments, the gene expression level and/or mRNA level of a target gene in a subject to whom a compound of Formula LP-4-a, LP-4-b, LP-18-a, LP-18-b, LP-128-a, LP-128-b, LP-151-a, LP-151-b, LP-183-a, LP-183-b, LP-200-a, LP-200-b, LP-208-a, LP-208-b, LP-211-a, LP-211-b, LP-232-a, LP-232-b, LP-242-a, LP-242-b, LP-243-a, LP-243-b, LP-244-a, LP-244-b, LP-245-a, LP-245-b, LP-249-a, LP-249-b, LP-274-a, LP-274-b, LP-295-a, LP-295-b, LP-310-a, LP-310-b, LP-359-a, LP-359-b, LP-361-a, LP-361-b, LP-371-a, LP-371-b, LP-374-a, LP-374-b, LP-375-a, LP-375-b, LP-377-a, LP-377-b, LP-378-a, LP-378-b, LP-379-a, LP-379-b, LP-380-a, LP-380-b, LP-403-a, LP-403-b, LP-404-a, LP-404-b, LP-412-a, LP-412-b, LP-413-a, LP-413-b, LP-416-a, LP-416-b, LP-424-a, LP-424-b, LP-425-a, LP-425-b, LP-426-a, LP-426-b, LP-427-a, LP-427-b, LP-428-a, LP-428-b, LP-432-a, LP-432-b, LP-433-a, LP-433-b, LP-444-a, LP-444-b, LP-445-a, LP-445-b, LP-446-a, LP-446-b, LP-447-a, LP-447-b, LP-453-a, LP-453-b, LP-455-a, LP-455-b, LP-457-a, LP-457-b, LP-458-a, LP-458-b, LP-459-a, LP-459-b, LP-460-a, LP-460-b, LP-461-a, LP-461-b, LP-468-a, LP-468-b, LP-469-a, LP-469-b, LP-470-a, LP-470-b, LP-473-a, LP-473-b, LP-474-a, LP-474-b, CNR1 SM2-a, or CNR1 SM2-b, described herein is administered is reduced by at least about 30%, 35%, 40%, 45%, 50%, 55%, 60%, 65%, 70%, 75%, 80%, 85%, 95%, 96%, 97%, 98%, 99%, or greater than 99% relative to the subject prior to being administered the compound or to a subject not receiving the compound. The gene expression level and/or mRNA level in the subject may be reduced in a cell, group of cells, and/or tissue of the subject.

In some embodiments, the target protein level in a subject to whom a compound of Formula LP-4-a, LP-4-b, LP-18-a, LP-18-b, LP-128-a, LP-128-b, LP-151-a, LP-151-b, LP-183-a, LP-183-b, LP-200-a, LP-200-b, LP-208-a, LP-208-b, LP-211-a, LP-211-b, LP-232-a, LP-232-b, LP-242-a, LP-242-b, LP-243-a, LP-243-b, LP-244-a, LP-244-b, LP-245-a, LP-245-b, LP-249-a, LP-249-b, LP-274-a, LP-274-b, LP-295-a, LP-295-b, LP-310-a, LP-310-b, LP-359-a, LP-359-b, LP-361-a, LP-361-b, LP-371-a, LP-371-b, LP-374-a, LP-374-b, LP-375-a, LP-375-b, LP-377-a, LP-377-b, LP-378-a, LP-378-b, LP-379-a, LP-379-b, LP-380-a, LP-380-b, LP-403-a, LP-403-b, LP-404-a, LP-404-b, LP-412-a, LP-412-b, LP-413-a, LP-413-b, LP-416-a, LP-416-b, LP-424-a, LP-424-b, LP-425-a, LP-425-b, LP-426-a, LP-426-b, LP-427-a, LP-427-b, LP-428-a, LP-428-b, LP-432-a, LP-432-b, LP-433-a, LP-433-b, LP-444-a, LP-444-b, LP-445-a, LP-445-b, LP-446-a, LP-446-b, LP-447-a, LP-447-b, LP-453-a, LP-453-b, LP-455-a, LP-455-b, LP-457-a, LP-457-b, LP-458-a, LP-458-b, LP-459-a, LP-459-b, LP-460-a, LP-460-b, LP-461-a, LP-461-b, LP-468-a, LP-468-b, LP-469-a, LP-469-b, LP-470-a, LP-470-b, LP-473-a, LP-473-b, LP-474-a, LP-474-b, CNR1 SM2-a, or CNR1 SM2-b, described herein has been administered is reduced by at least about 30%, 35%, 40%, 45%, 50%, 55%, 60%, 65%, 70%, 75%, 80%, 85%, 90%, 95%, 96%, 97%, 98%, 99%, or greater than 99% relative to the subject prior to being administered the compound or to a subject not receiving the compound. The protein level in the subject may be reduced in a cell, group of cells, tissue, blood, and/or other fluid of the subject.

A reduction in target mRNA levels and/or target protein levels can be assessed by any methods known in the art. As used herein, a reduction or decrease in target mRNA level and/or protein level are collectively referred to herein as a reduction or decrease in target gene and/or protein levels or inhibiting or reducing the expression of a target gene.

In some embodiments, compounds of Formula LP-4-a, LP-4-b, LP-18-a, LP-18-b, LP-128-a, LP-128-b, LP-151-a, LP-151-b, LP-183-a, LP-183-b, LP-200-a, LP-200-b, LP-208-a, LP-208-b, LP-211-a, LP-211-b, LP-232-a, LP-232-b, LP-242-a, LP-242-b, LP-243-a, LP-243-b, LP-244-a, LP-244-b, LP-245-a, LP-245-b, LP-249-a, LP-249-b, LP-274-a, LP-274-b, LP-295-a, LP-295-b, LP-310-a, LP-310-b, LP-359-a, LP-359-b, LP-361-a, LP-361-b, LP-371-a, LP-371-b, LP-374-a, LP-374-b, LP-375-a, LP-375-b, LP-377-a, LP-377-b, LP-378-a, LP-378-b, LP-379-a, LP-379-b, LP-380-a, LP-380-b, LP-403-a, LP-403-b, LP-404-a, LP-404-b, LP-412-a, LP-412-b, LP-413-a, LP-413-b, LP-416-a, LP-416-b, LP-424-a, LP-424-b, LP-425-a, LP-425-b, LP-426-a, LP-426-b, LP-427-a, LP-427-b, LP-428-a, LP-428-b, LP-432-a, LP-432-b, LP-433-a, LP-433-b, LP-444-a, LP-444-b, LP-445-a, LP-445-b, LP-446-a, LP-446-b, LP-447-a, LP-447-b, LP-453-a, LP-453-b, LP-455-a, LP-455-b, LP-457-a, LP-457-b, LP-458-a, LP-458-b, LP-459-a, LP-459-b, LP-460-a, LP-460-b, LP-461-a, LP-461-b, LP-468-a, LP-468-b, LP-469-a, LP-469-b, LP-470-a, LP-470-b, LP-473-a, LP-473-b, LP-474-a, LP-474-b, CNR1 SM2-a, or CNR1 SM2-b, described herein may be used in the preparation of a pharmaceutical composition for use in the treatment of a disease, disorder, or symptom that is mediated at least in part by target gene expression. In some embodiments, the disease, disorder, or symptom that is mediated at least in part by target gene expression is an adipose disease or disorder.

In some embodiments, methods of treating a subject are dependent on the body weight of the subject. In some embodiments, compounds of Formula LP-4-a, LP-4-b, LP-18-a, LP-18-b, LP-128-a, LP-128-b, LP-151-a, LP-151-b, LP-183-a, LP-183-b, LP-200-a, LP-200-b, LP-208-a, LP-208-b, LP-211-a, LP-211-b, LP-232-a, LP-232-b, LP-242-a, LP-242-b, LP-243-a, LP-243-b, LP-244-a, LP-244-b, LP-245-a, LP-245-b, LP-249-a, LP-249-b, LP-274-a, LP-274-b, LP-295-a, LP-295-b, LP-310-a, LP-310-b, LP-359-a, LP-359-b, LP-361-a, LP-361-b, LP-371-a, LP-371-b, LP-374-a, LP-374-b, LP-375-a, LP-375-b, LP-377-a, LP-377-b, LP-378-a, LP-378-b, LP-379-a, LP-379-b, LP-380-a, LP-380-b, LP-403-a, LP-403-b, LP-404-a, LP-404-b, LP-412-a, LP-412-b, LP-413-a, LP-413-b, LP-416-a, LP-416-b, LP-424-a, LP-424-b, LP-425-a, LP-425-b, LP-426-a, LP-426-b, LP-427-a, LP-427-b, LP-428-a, LP-428-b, LP-432-a, LP-432-b, LP-433-a, LP-433-b, LP-444-a, LP-444-b, LP-445-a, LP-445-b, LP-446-a, LP-446-b, LP-447-a, LP-447-b, LP-453-a, LP-453-b, LP-455-a, LP-455-b, LP-457-a, LP-457-b, LP-458-a, LP-458-b, LP-459-a, LP-459-b, LP-460-a, LP-460-b, LP-461-a, LP-461-b, LP-468-a, LP-468-b, LP-469-a, LP-469-b, LP-470-a, LP-470-b, LP-473-a, LP-473-b, LP-474-a, LP-474-b, CNR1 SM2-a, or CNR1 SM2-b, may be administered at a dose of about 0.05 mg/kg to about 40.0 mg/kg of body weight of the subject. In other embodiments compounds of Formula LP-4-a, LP-4-b, LP-18-a, LP-18-b, LP-128-a, LP-128-b, LP-151-a, LP-151-b, LP-183-a, LP-183-b, LP-200-a, LP-200-b, LP-208-a, LP-208-b, LP-211-a, LP-211-b, LP-232-a, LP-232-b, LP-242-a, LP-242-b, LP-243-a, LP-243-b, LP-244-a, LP-244-b, LP-245-a, LP-245-b, LP-249-a, LP-249-b, LP-274-a, LP-274-b, LP-295-a, LP-295-b, LP-310-a, LP-310-b, LP-359-a, LP-359-b, LP-361-a, LP-361-b, LP-371-a, LP-371-b, LP-374-a, LP-374-b, LP-375-a, LP-375-b, LP-377-a, LP-377-b, LP-378-a, LP-378-b, LP-379-a, LP-379-b, LP-380-a, LP-380-b, LP-403-a, LP-403-b, LP-404-a, LP-404-b, LP-412-a, LP-412-b, LP-413-a, LP-413-b, LP-416-a, LP-416-b, LP-424-a, LP-424-b, LP-425-a, LP-425-b, LP-426-a, LP-426-b, LP-427-a, LP-427-b, LP-428-a, LP-428-b, LP-432-a, LP-432-b, LP-433-a, LP-433-b, LP-444-a, LP-444-b, LP-445-a, LP-445-b, LP-446-a, LP-446-b, LP-447-a, LP-447-b, LP-453-a, LP-453-b, LP-455-a, LP-455-b, LP-457-a, LP-457-b, LP-458-a, LP-458-b, LP-459-a, LP-459-b, LP-460-a, LP-460-b, LP-461-a, LP-461-b, LP-468-a, LP-468-b, LP-469-a, LP-469-b, LP-470-a, LP-470-b, LP-473-a, LP-473-b, LP-474-a, LP-474-b, CNR1 SM2-a, or CNR1 SM2-b, may be administered at a dose of about 5 mg/kg to about 20 mg/kg of body weight of the subject.

In some embodiments, compounds of Formula LP-4-a, LP-4-b, LP-18-a, LP-18-b, LP-128-a, LP-128-b, LP-151-a, LP-151-b, LP-183-a, LP-183-b, LP-200-a, LP-200-b, LP-208-a, LP-208-b, LP-211-a, LP-211-b, LP-232-a, LP-232-b, LP-242-a, LP-242-b, LP-243-a, LP-243-b, LP-244-a, LP-244-b, LP-245-a, LP-245-b, LP-249-a, LP-249-b, LP-274-a, LP-274-b, LP-295-a, LP-295-b, LP-310-a, LP-310-b, LP-359-a, LP-359-b, LP-361-a, LP-361-b, LP-371-a, LP-371-b, LP-374-a, LP-374-b, LP-375-a, LP-375-b, LP-377-a, LP-377-b, LP-378-a, LP-378-b, LP-379-a, LP-379-b, LP-380-a, LP-380-b, LP-403-a, LP-403-b, LP-404-a, LP-404-b, LP-412-a, LP-412-b, LP-413-a, LP-413-b, LP-416-a, LP-416-b, LP-424-a, LP-424-b, LP-425-a, LP-425-b, LP-426-a, LP-426-b, LP-427-a, LP-427-b, LP-428-a, LP-428-b, LP-432-a, LP-432-b, LP-433-a, LP-433-b, LP-444-a, LP-444-b, LP-445-a, LP-445-b, LP-446-a, LP-446-b, LP-447-a, LP-447-b, LP-453-a, LP-453-b, LP-455-a, LP-455-b, LP-457-a, LP-457-b, LP-458-a, LP-458-b, LP-459-a, LP-459-b, LP-460-a, LP-460-b, LP-461-a, LP-461-b, LP-468-a, LP-468-b, LP-469-a, LP-469-b, LP-470-a, LP-470-b, LP-473-a, LP-473-b, LP-474-a, LP-474-b, CNR1 SM2-a, or CNR1 SM2-b, may be administered in a split dose, meaning that two doses are given to a subject in a short (for example, less than 24 hour) time period. In some embodiments, about half of the desired daily amount is administered in an initial administration, and the remaining about half of the desired daily amount is administered approximately four hours after the initial administration.

In some embodiments, compounds of Formula LP-4-a, LP-4-b, LP-18-a, LP-18-b, LP-128-a, LP-128-b, LP-151-a, LP-151-b, LP-183-a, LP-183-b, LP-200-a, LP-200-b, LP-208-a, LP-208-b, LP-211-a, LP-211-b, LP-232-a, LP-232-b, LP-242-a, LP-242-b, LP-243-a, LP-243-b, LP-244-a, LP-244-b, LP-245-a, LP-245-b, LP-249-a, LP-249-b, LP-274-a, LP-274-b, LP-295-a, LP-295-b, LP-310-a, LP-310-b, LP-359-a, LP-359-b, LP-361-a, LP-361-b, LP-371-a, LP-371-b, LP-374-a, LP-374-b, LP-375-a, LP-375-b, LP-377-a, LP-377-b, LP-378-a, LP-378-b, LP-379-a, LP-379-b, LP-380-a, LP-380-b, LP-403-a, LP-403-b, LP-404-a, LP-404-b, LP-412-a, LP-412-b, LP-413-a, LP-413-b, LP-416-a, LP-416-b, LP-424-a, LP-424-b, LP-425-a, LP-425-b, LP-426-a, LP-426-b, LP-427-a, LP-427-b, LP-428-a, LP-428-b, LP-432-a, LP-432-b, LP-433-a, LP-433-b, LP-444-a, LP-444-b, LP-445-a, LP-445-b, LP-446-a, LP-446-b, LP-447-a, LP-447-b, LP-453-a, LP-453-b, LP-455-a, LP-455-b, LP-457-a, LP-457-b, LP-458-a, LP-458-b, LP-459-a, LP-459-b, LP-460-a, LP-460-b, LP-461-a, LP-461-b, LP-468-a, LP-468-b, LP-469-a, LP-469-b, LP-470-a, LP-470-b, LP-473-a, LP-473-b, LP-474-a, LP-474-b, CNR1 SM2-a, or CNR1 SM2-b, described herein may be administered daily (i.e., once each day). In some embodiments, compounds of Formula LP-4-a, LP-4-b, LP-18-a, LP-18-b, LP-128-a, LP-128-b, LP-151-a, LP-151-b, LP-183-a, LP-183-b, LP-200-a, LP-200-b, LP-208-a, LP-208-b, LP-211-a, LP-211-b, LP-232-a, LP-232-b, LP-242-a, LP-242-b, LP-243-a, LP-243-b, LP-244-a, LP-244-b, LP-245-a, LP-245-b, LP-249-a, LP-249-b, LP-274-a, LP-274-b, LP-295-a, LP-295-b, LP-310-a, LP-310-b, LP-359-a, LP-359-b, LP-361-a, LP-361-b, LP-371-a, LP-371-b, LP-374-a, LP-374-b, LP-375-a, LP-375-b, LP-377-a, LP-377-b, LP-378-a, LP-378-b, LP-379-a, LP-379-b, LP-380-a, LP-380-b, LP-403-a, LP-403-b, LP-404-a, LP-404-b, LP-412-a, LP-412-b, LP-413-a, LP-413-b, LP-416-a, LP-416-b, LP-424-a, LP-424-b, LP-425-a, LP-425-b, LP-426-a, LP-426-b, LP-427-a, LP-427-b, LP-428-a, LP-428-b, LP-432-a, LP-432-b, LP-433-a, LP-433-b, LP-444-a, LP-444-b, LP-445-a, LP-445-b, LP-446-a, LP-446-b, LP-447-a, LP-447-b, LP-453-a, LP-453-b, LP-455-a, LP-455-b, LP-457-a, LP-457-b, LP-458-a, LP-458-b, LP-459-a, LP-459-b, LP-460-a, LP-460-b, LP-461-a, LP-461-b, LP-468-a, LP-468-b, LP-469-a, LP-469-b, LP-470-a, LP-470-b, LP-473-a, LP-473-b, LP-474-a, LP-474-b, CNR1 SM2-a, or CNR1 SM2-b, described herein may be administered once a week (i.e., weekly). In other embodiments, compounds of LP-4-a, LP-4-b, LP-18-a, LP-18-b, LP-128-a, LP-128-b, LP-151-a, LP-151-b, LP-183-a, LP-183-b, LP-200-a, LP-200-b, LP-208-a, LP-208-b, LP-211-a, LP-211-b, LP-232-a, LP-232-b, LP-242-a, LP-242-b, LP-243-a, LP-243-b, LP-244-a, LP-244-b, LP-245-a, LP-245-b, LP-249-a, LP-249-b, LP-274-a, LP-274-b, LP-295-a, LP-295-b, LP-310-a, LP-310-b, LP-359-a, LP-359-b, LP-361-a, LP-361-b, LP-371-a, LP-371-b, LP-374-a, LP-374-b, LP-375-a, LP-375-b, LP-377-a, LP-377-b, LP-378-a, LP-378-b, LP-379-a, LP-379-b, LP-380-a, LP-380-b, LP-403-a, LP-403-b, LP-404-a, LP-404-b, LP-412-a, LP-412-b, LP-413-a, LP-413-b, LP-416-a, LP-416-b, LP-424-a, LP-424-b, LP-425-a, LP-425-b, LP-426-a, LP-426-b, LP-427-a, LP-427-b, LP-428-a, LP-428-b, LP-432-a, LP-432-b, LP-433-a, LP-433-b, LP-444-a, LP-444-b, LP-445-a, LP-445-b, LP-446-a, LP-446-b, LP-447-a, LP-447-b, LP-453-a, LP-453-b, LP-455-a, LP-455-b, LP-457-a, LP-457-b, LP-458-a, LP-458-b, LP-459-a, LP-459-b, LP-460-a, LP-460-b, LP-461-a, LP-461-b, LP-468-a, LP-468-b, LP-469-a, LP-469-b, LP-470-a, LP-470-b, LP-473-a, LP-473-b, LP-474-a, LP-474-b, CNR1 SM2-a, or CNR1 SM2-b described herein may be administered biweekly (once every other week).

In some embodiments, compounds of Formula LP-4-a, LP-4-b, LP-18-a, LP-18-b, LP-128-a, LP-128-b, LP-151-a, LP-151-b, LP-183-a, LP-183-b, LP-200-a, LP-200-b, LP-208-a, LP-208-b, LP-211-a, LP-211-b, LP-232-a, LP-232-b, LP-242-a, LP-242-b, LP-243-a, LP-243-b, LP-244-a, LP-244-b, LP-245-a, LP-245-b, LP-249-a, LP-249-b, LP-274-a, LP-274-b, LP-295-a, LP-295-b, LP-310-a, LP-310-b, LP-359-a, LP-359-b, LP-361-a, LP-361-b, LP-371-a, LP-371-b, LP-374-a, LP-374-b, LP-375-a, LP-375-b, LP-377-a, LP-377-b, LP-378-a, LP-378-b, LP-379-a, LP-379-b, LP-380-a, LP-380-b, LP-403-a, LP-403-b, LP-404-a, LP-404-b, LP-412-a, LP-412-b, LP-413-a, LP-413-b, LP-416-a, LP-416-b, LP-424-a, LP-424-b, LP-425-a, LP-425-b, LP-426-a, LP-426-b, LP-427-a, LP-427-b, LP-428-a, LP-428-b, LP-432-a, LP-432-b, LP-433-a, LP-433-b, LP-444-a, LP-444-b, LP-445-a, LP-445-b, LP-446-a, LP-446-b, LP-447-a, LP-447-b, LP-453-a, LP-453-b, LP-455-a, LP-455-b, LP-457-a, LP-457-b, LP-458-a, LP-458-b, LP-459-a, LP-459-b, LP-460-a, LP-460-b, LP-461-a, LP-461-b, LP-468-a, LP-468-b, LP-469-a, LP-469-b, LP-470-a, LP-470-b, LP-473-a, LP-473-b, LP-474-a, LP-474-b, CNR1 SM2-a, or CNR1 SM2-b described herein or compositions containing such compounds may be used for the treatment of a disease, disorder, or symptom that is mediated at least in part by target gene expression. In some embodiments, the disease, disorder or symptom that is mediated at least in part by target gene expression is an adipose disease or disorder.

Another aspect of the invention provides for a method of reducing a target gene expression in vivo, the method comprising introducing to a cell a compound of Formula LP-4-a, LP-4-b, LP-18-a, LP-18-b, LP-128-a, LP-128-b, LP-151-a, LP-151-b, LP-183-a, LP-183-b, LP-200-a, LP-200-b, LP-208-a, LP-208-b, LP-211-a, LP-211-b, LP-232-a, LP-232-b, LP-242-a, LP-242-b, LP-243-a, LP-243-b, LP-244-a, LP-244-b, LP-245-a, LP-245-b, LP-249-a, LP-249-b, LP-274-a, LP-274-b, LP-295-a, LP-295-b, LP-310-a, LP-310-b, LP-359-a, LP-359-b, LP-361-a, LP-361-b, LP-371-a, LP-371-b, LP-374-a, LP-374-b, LP-375-a, LP-375-b, LP-377-a, LP-377-b, LP-378-a, LP-378-b, LP-379-a, LP-379-b, LP-380-a, LP-380-b, LP-403-a, LP-403-b, LP-404-a, LP-404-b, LP-412-a, LP-412-b, LP-413-a, LP-413-b, LP-416-a, LP-416-b, LP-424-a, LP-424-b, LP-425-a, LP-425-b, LP-426-a, LP-426-b, LP-427-a, LP-427-b, LP-428-a, LP-428-b, LP-432-a, LP-432-b, LP-433-a, LP-433-b, LP-444-a, LP-444-b, LP-445-a, LP-445-b, LP-446-a, LP-446-b, LP-447-a, LP-447-b, LP-453-a, LP-453-b, LP-455-a, LP-455-b, LP-457-a, LP-457-b, LP-458-a, LP-458-b, LP-459-a, LP-459-b, LP-460-a, LP-460-b, LP-461-a, LP-461-b, LP-468-a, LP-468-b, LP-469-a, LP-469-b, LP-470-a, LP-470-b, LP-473-a, LP-473-b, LP-474-a, LP-474-b, CNR1 SM2-a, or CNR1 SM2-b described herein, wherein the compound comprises an oligonucleotide-based agent at least substantially complementary to the target gene. In some embodiments, the cell is an adipocyte. In some embodiments, the cell is within a subject. In some embodiments, the subject has been diagnosed with a disease or disorder that is treated, prevented or ameliorated by reducing expression of the target gene.

Another aspect of the invention provides for the use of any one of the lipid PK/PD modulators conjugated to an oligonucleotide-based agent described herein for the treatment, prevention, or amelioration of a disease or disorder. In some embodiments, the disease or disorder is an adipose-related disease (such as such as lipodystrophy, lipedema, etc) or disorder selected from the group consisting of obesity, type 2 diabetes mellitus, insulin resistance, metabolic syndrome, various lipodystrophies, atherosclerotic vascular disease, cardiometabolic diseases or disorders, or other similar diseases or disorders.

### Cells, Tissues, and Non-Human Organisms

Cells, tissues, and non-human organisms that include at least one of the compounds of Formula LP-4-a, LP-4-b, LP-18-a, LP-18-b, LP-128-a, LP-128-b, LP-151-a, LP-151-b, LP-183-a, LP-183-b, LP-200-a, LP-200-b, LP-208-a, LP-208-b, LP-211-a, LP-211-b, LP-232-a, LP-232-b, LP-242-a, LP-242-b, LP-243-a, LP-243-b, LP-244-a, LP-244-b, LP-245-a, LP-245-b, LP-249-a, LP-249-b, LP-274-a, LP-274-b, LP-295-a, LP-295-b, LP-310-a, LP-310-b, LP-359-a, LP-359-b, LP-361-a, LP-361-b, LP-371-a, LP-371-b, LP-374-a, LP-374-b, LP-375-a, LP-375-b, LP-377-a, LP-377-b, LP-378-a, LP-378-b, LP-379-a, LP-379-b, LP-380-a, LP-380-b, LP-403-a, LP-403-b, LP-404-a, LP-404-b, LP-412-a, LP-412-b, LP-413-a, LP-413-b, LP-416-a, LP-416-b, LP-424-a, LP-424-b, LP-425-a, LP-425-b, LP-426-a, LP-426-b, LP-427-a, LP-427-b, LP-428-a, LP-428-b, LP-432-a, LP-432-b, LP-433-a, LP-433-b, LP-444-a, LP-444-b, LP-445-a, LP-445-b, LP-446-a, LP-446-b, LP-447-a, LP-447-b, LP-453-a, LP-453-b, LP-455-a, LP-455-b, LP-457-a, LP-457-b, LP-458-a, LP-458-b, LP-459-a, LP-459-b, LP-460-a, LP-460-b, LP-461-a, LP-461-b, LP-468-a, LP-468-b, LP-469-a, LP-469-b, LP-470-a, LP-470-b, LP-473-a, LP-473-b, LP-474-a, LP-474-b, CNR1 SM2-a, or CNR1 SM2-b described herein is contemplated. The cell, tissue, or non-human organism is made by delivering the compound of Formula LP-4-a, LP-4-b, LP-18-a, LP-18-b, LP-128-a, LP-128-b, LP-151-a, LP-151-b, LP-183-a, LP-183-b, LP-200-a, LP-200-b, LP-208-a, LP-208-b, LP-211-a, LP-211-b, LP-232-a, LP-232-b, LP-242-a, LP-242-b, LP-243-a, LP-243-b, LP-244-a, LP-244-b, LP-245-a, LP-245-b, LP-249-a, LP-249-b, LP-274-a, LP-274-b, LP-295-a, LP-295-b, LP-310-a, LP-310-b, LP-359-a, LP-359-b, LP-361-a, LP-361-b, LP-371-a, LP-371-b, LP-374-a, LP-374-b, LP-375-a, LP-375-b, LP-377-a, LP-377-b, LP-378-a, LP-378-b, LP-379-a, LP-379-b, LP-380-a, LP-380-b, LP-403-a, LP-403-b, LP-404-a, LP-404-b, LP-412-a, LP-412-b, LP-413-a, LP-413-b, LP-416-a, LP-416-b, LP-424-a, LP-424-b, LP-425-a, LP-425-b, LP-426-a, LP-426-b, LP-427-a, LP-427-b, LP-428-a, LP-428-b, LP-432-a, LP-432-b, LP-433-a, LP-433-b, LP-444-a, LP-444-b, LP-445-a, LP-445-b, LP-446-a, LP-446-b, LP-447-a, LP-447-b, LP-453-a, LP-453-b, LP-455-a, LP-455-b, LP-457-a, LP-457-b, LP-458-a, LP-458-b, LP-459-a, LP-459-b, LP-460-a, LP-460-b, LP-461-a, LP-461-b, LP-468-a, LP-468-b, LP-469-a, LP-469-b, LP-470-a, LP-470-b, LP-473-a, LP-473-b, LP-474-a, LP-474-b, CNR1 SM2-a, or CNR1 SM2-b to the cell, tissue, or non-human organism by any means available in the art. In some embodiments, the cell is a mammalian cell, including, but not limited to, a human cell. In some embodiments the cell is an adipocyte.

The above provided embodiments and items are now illustrated with the following, non-limiting examples.

### EXAMPLES

The following examples are not limiting and are intended to illustrate certain embodiments disclosed herein.

Unless expressly stated otherwise, numerals used to refer to compounds of a given example are only made with reference to that particular example and not any other examples disclosed herein. For example, compound 1 of “Synthesis of LP-4-p phosphoramidite” in Example 2 is different from, and does not refer to, compound 1 of “Synthesis of LP-18-p” in Example 2. Similarly, it will be appreciated that a particular compound disclosed herein may be identified by different numerals in different examples. Compounds that are disclosed in various tables throughout the detailed description (i.e., LPXXa, LPXXb, and LPXX-p, wherein XX is a number) are referred to consistently throughout the examples herein.

It will be appreciated that, unless expressly stated otherwise, use of the term “EDC” in the examples herein refers to the N-(3-Dimethylaminopropyl)-N′-ethylcarbodiimide hydrochloride salt which is commercially available.

### Example 1. Syntheses of RNAi Agents and Compositions

The following describes the general procedures for the syntheses of oligonucleotide-based agents, such as RNAi agents and antisense oligonucleotides, and conjugates thereof, that are illustrated in the non-limiting Examples set forth herein.

Synthesis of Oligonucleotide-based Agents. Oligonucleotide-based agents can be synthesized using methods generally known in the art. For the synthesis of the RNAi agents illustrated in the Examples set forth herein, the sense and antisense strands of the RNAi agents were synthesized according to solid phase phosphoramidite technology used in oligonucleotide synthesis. Depending on the scale, a MerMade96E® (Bioautomation), a MerMadel2® (Bioautomation), or an Oligopilot 100 (GE Healthcare) was used. Syntheses were performed on a solid support made of controlled pore glass (CPG, 500 Å or 600 Å, obtained from Prime Synthesis, Aston, PA, USA) or polystyrene (obtained from Kinovate, Oceanside, CA, USA). All RNA and 2′-modified RNA phosphoramidites were purchased from Thermo Fisher Scientific (Milwaukee, WI, USA), ChemGenes (Wilmington, MA, USA), or Hongene Biotech (Morrisville, NC, USA). Specifically, the following 2′-O-methyl phosphoramidites that were used include the following: (5′-O-dimethoxytrityl-N⁶-(benzoyl)-2′-O-methyl-adenosine-3′-O-(2-cyanoethyl-N,N-diisopropylamino) phosphoramidite, 5′-O-dimethoxy-trityl-N⁴-(acetyl)-2′-O-methyl-cytidine-3′-O-(2-cyanoethyl-N,N-diisopropyl-amino) phosphoramidite, (5′-O-dimethoxytrityl-N²-(isobutyryl)-2′-O-methyl-guanosine-3′-O-(2-cyanoethyl-N,N-diisopropylamino) phosphoramidite, and 5′-O-dimethoxytrityl-2′-O-methyl-uridine-3′-O-(2-cyanoethyl-N,N-diisopropylamino) phosphoramidite. The 2′-deoxy-2′-fluoro-phosphoramidites and 2′-O-propargyl phosphoramidites carried the same protecting groups as the 2′-O-methyl phosphoramidites. 5′-dimethoxytrityl-2′-O-methyl-inosine-3′-O-(2-cyanoethyl-N,N-diisopropylamino) phosphoramidites were purchased from Glen Research (Virginia). The inverted abasic (3′-O-dimethoxytrityl-2′-deoxyribose-5′-O-(2-cyanoethyl-N,N-diisopropylamino) phosphoramidites were purchased from ChemGenes. The following UNA phosphoramidites that were used included the following: 5′-(4,4′-Dimethoxytrityl)-N6-(benzoyl)-2′,3′-seco-adenosine, 2′-benzoyl-3′-[(2-cyanoethyl)-(N,N-diisopropyl)]-phosphoramidite, 5′-(4,4′-Dimethoxytrityl)-N-acetyl-2′,3′-seco-cytosine, 2′-benzoyl-3′-[(2-cyanoethyl)-(N,N-diiso-propyl)]-phosphoramidite, 5′-(4,4′-Dimethoxytrityl)-N-isobutyryl-2′,3′-seco-guanosine, 2′-benzoyl-3′-[(2-cyanoethyl)-(N,N-diisopropyl)]-phosphoramidite, and 5′-(4,4′-Dimethoxy-trityl)-2′,3′-seco-uridine, 2′-benzoyl-3′-[(2-cyanoethyl)-(N,N-di-iso-propyl)]-phosphoramidite. In order to introduce phosphorothioate linkages, a 100 mM solution of 3-phenyl 1,2,4-dithiazoline-5-one (POS, obtained from PolyOrg, Inc., Leominster, MA, USA) in anhydrous acetonitrile or a 200 mM solution of xanthane hydride (TCI America, Portland, OR, USA) in pyridine was employed. Antisense oligonucleotides may be manufactured using the same general methods as those used in connection with the RNAi agent conjugates described in the Examples herein.

TFA aminolink phosphoramidites were also commercially purchased (ThermoFisher). Linker L6 was purchased as propargyl-PEG5-NHS from BroadPharm (catalog #BP-20907) and coupled to the NH₂—C₆ group from an aminolink phosphoramidite to form -L6-C6-, using standard coupling conditions. The linker Alk-cyHex was similarly commercially purchased from Lumiprobe (alkyne phosphoramidite, 5′-terminal) as a propargyl-containing compound phosphoramidite compound to form the linker -Alk-cyHex-. In each case, phosphorothioate linkages were introduced as specified using the conditions set forth herein. The cyclopropyl phosphonate phosphoramidites were synthesized in accordance with International Patent Application Publication No. WO 2017/214112 (see also Altenhofer et. al., Chem. Communications (Royal Soc. Chem.), 57(55):6808-6811 (July 2021)).

For some RNAi agents disclosed herein, a linker, such as a C6-SS-C6 or a 6-SS-6 group, was introduced at the 3′ terminal end of the sense strand. Pre-loaded resin was commercially acquired with the respective linker. Alternatively, for some sense strands, a dT resin was used and the respectively linker was then added via standard phosphoramidite synthesis.

Cleavage and deprotection of support bound oligomer. After finalization of the solid phase synthesis, the dried solid support was treated with a 1:1 volume solution of 40 weight (wt.) % methylamine in water and 28% to 31% ammonium hydroxide solution (Aldrich) for 1.5 hours at 30° C. The solution was evaporated and the solid residue was reconstituted in water (see below).

Purification. Crude oligomers were purified by anionic exchange HPLC using a TSKgel® SuperQ-5PW 13 μm column (available from Tosoh Biosciences) and Shimadzu LC-8 system. Buffer A was 20 mM Tris, 5 mM EDTA, pH 9.0 and contained 20% Acetonitrile and buffer B was the same as buffer A with the addition of 1.5 M sodium chloride. UV traces at 260 nm were recorded. Appropriate fractions were pooled then run on size exclusion HPLC using a GE Healthcare XK 16/40 column packed with Sephadex® G25 fine (available from Sigma Aldrich) with a running buffer of 100 mM ammonium bicarbonate, pH 6.7 and 20% Acetonitrile or filtered water. Alternatively, pooled fractions were desalted and exchanged into an appropriate buffer or solvent system via tangential flow filtration.

Annealing. For the RNAi agents disclosed in the Examples herein, complementary strands were mixed by combining equimolar RNA solutions (sense and antisense) in 1×PBS (Phosphate-Buffered Saline, 1×, Corning, Cellgro) to form the RNAi agents. Some RNAi agents were lyophilized and stored at −15 to −25° C. Duplex concentration was determined by measuring the solution absorbance on a UV-Vis spectrometer in 1×PBS. The solution absorbance at 260 nm was then multiplied by a conversion factor and the dilution factor to determine the duplex concentration. The conversion factor used was either 0.037 mg/(mL·cm) or was calculated from an experimentally determined extinction coefficient.

### Example 2. Synthesis of Lipid PK/PD Modulator Precursors

### Synthesis of LP-4-p

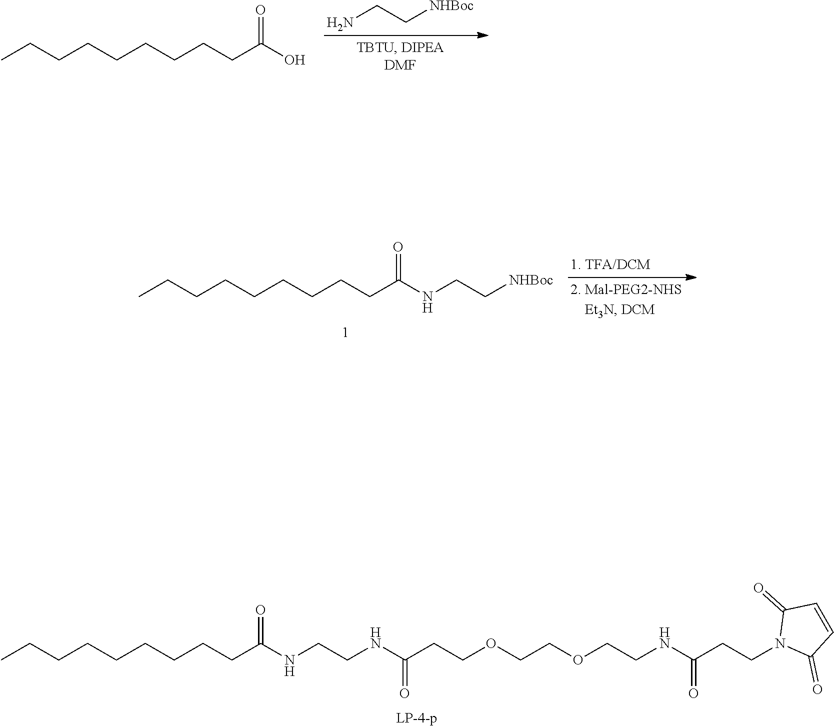 [SMILES: CCCCCCCCCC(=O)O ; CC(C)(C)OC(=O)NCCN ; CCCCCCCCCC(=O)NCCNC(=O)OC(C)(C)C ; CCCCCCCCCC(=O)NCCNC(=O)CCOCCOCCNC(=O)CCN1C(=O)C=CC1=O]

Decanoic acid (182 mg, 1.06 mmol) was stirred in DMF (5 mL). N-boc-Ethylenediamine (0.185 mL, 1.16 mmol) was added followed by TBTU (409 mg, 1.27 mmol) and DIPEA (0.555 mL, 3.18 mmol). The suspension was stirred for 16 h and then the reaction was diluted with H₂O and extracted with EtOAc. The organic layer was dried over Na₂SO₄, filtered, and concentration to dryness. The crude product was purified by flash chromatography (EtOAc/hexanes) to give 1 (232 mg). To intermediate 1 (232 mg, 0.738 mmol) was added TFA:DCM (1:1) and the reaction was stirred until completion was confirmed by LCMS. The reaction was concentrated, the residue was dissolved in DCM (5 mL), and maleimide-PEG₂-NHS ester (300 mg, 0.848 mmol) and DIPEA (0.369 mL, 2.12 mmol) were added. The reaction was stirred overnight and then the reaction was diluted with H2O, extracted with EtOAc, dried over Na₂SO₄, filtered, and concentrated. Crude product was purified with flash chromatography (EtOAc/hexanes) to give LP-4-p. LC/MS (ESI+) calculated m/z 524.32 (M), found 524.65 (M+H+).

### Synthesis of LP-18-p

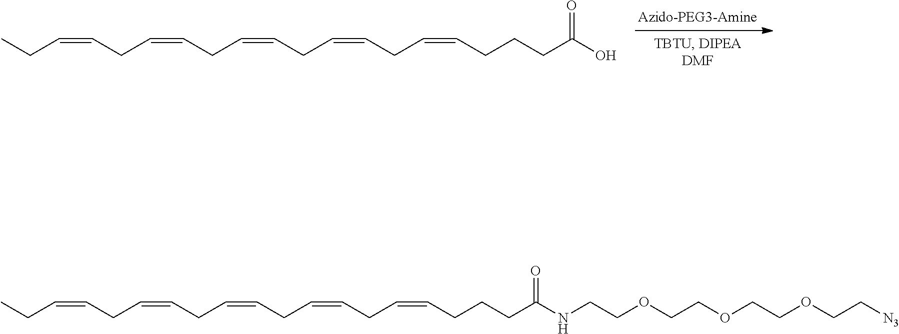 [SMILES: CC/C=C\C/C=C\C/C=C\C/C=C\C/C=C\CCCC(=O)O ; CC/C=C\C/C=C\C/C=C\C/C=C\C/C=C\CCCC(=O)NCCOCCOCCOCCN=[N+]=[N-]]

Arachidonic acid (188 mg, 0.619 mmol) was dissolved in DMF (5 mL) and then azido-PEG₃-amine (148 mg, 0.682 mmol), TBTU (238 mg, 0.743 mmol), and DIPEA (0.324 mL, 1.85 mmol) were added and the suspension was stirred overnight. The reaction was diluted with H₂O and extracted with DCM containing 20% trifluoroethanol. The extracts were dried over Na₂SO₄, filtered, and concentrated to dryness to give crude product that was purified by flash chromatography to give LP-18-p. LC/MS (ESI+) calculated m/z 502.35 (M), found 504.71 (M+H+).

### Synthesis of LP-151-p

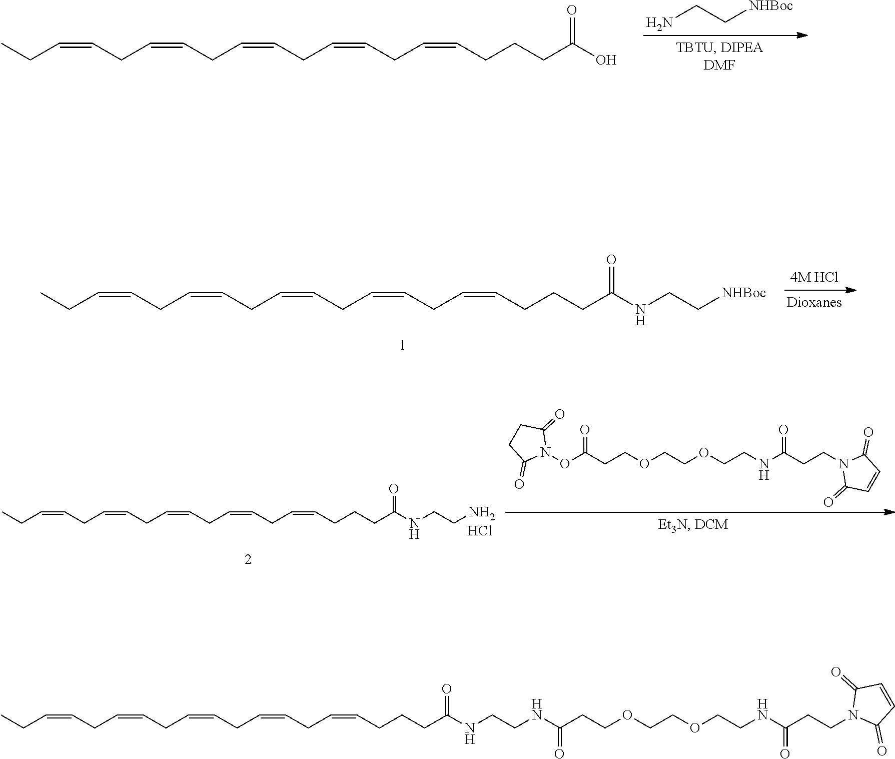

Intermediate 2: To a solution of eicosapentaenoic acid (100 mg, 0.330 mmol) in DMF was added N-boc-ethylenediamine 53 mg, 0.330 mmol), TBTU (106 mg, 0330 mmol), and DIPEA (0.23 mL, 1.32 mmol). The reaction mixture was stirred until full conversion seen by LCMS. The reaction was extracted with EtOAc and washed with saturated NaHCO₃, H₂O, and saturated NH₄Cl. The organic layer was dried over Na₂SO₄, filtered, and concentrated. The residue was purified with flash chromatography using silica gel as stationary phase (0-20% MeOH/DCM) to give 1 (147 mg) as a white oily residue in 65% yield. LC/MS calculated [M+H] 445.34 m/z, observed 445.56. To compound 1 (96 mg, 0.216 mmol) was added 4 M HCl in dioxane (10 equiv.) and the reaction was stirred until full conversion was seen in LCMS. The reaction was azeotroped with PhMe/MeOH and concentrated to give 2 as a white solid. LC/MS calculated [M+H] 345.28 m/z, observed 345.59.

A solution of 2 (82.5 mg, 0.216 mmol) and triethylamine (0.151 mL, 0.109 mmol) in dry DCM was stirred under N2. Maleimide-amido-PEG2-NHS ester (92.1 mg, 0.216 mmol) was slowly added and the reaction was stirred until full conversion was observed by LCMS. The reaction was washed with saturated NaHCO₃ and NH₄Cl, dried over Na₂SO₄, filtered, and concentrated. The crude residue was purified with flash chromatography using a 12 g silica gel column (0-8% MeOH/DCM over 20 min, product eluted around 7% MeOH) to give LP-151-p (141 mg) in 46% yield. LC/MS calculated [M+H] 655.40 m/z, observed 656.06.

### Synthesis of LP-208-p

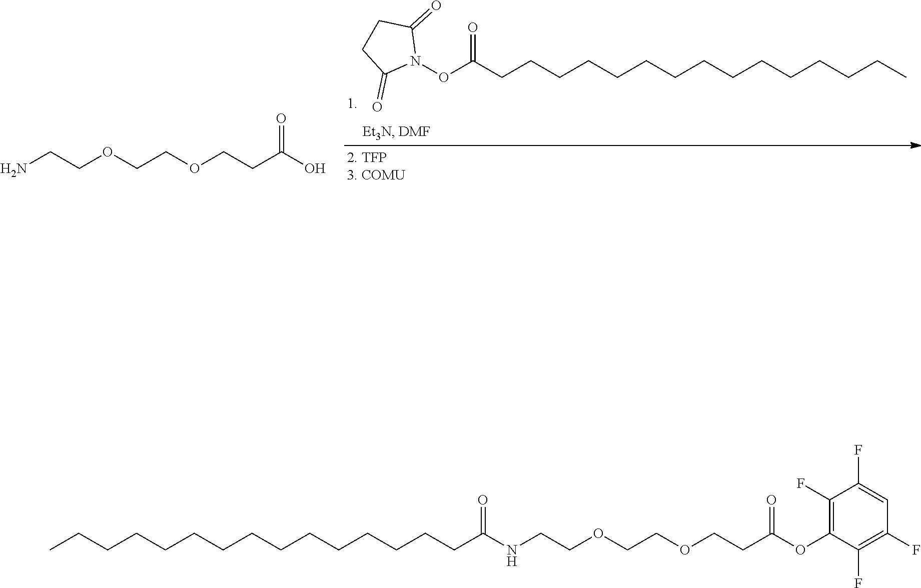

To the solution of amine-PEG₂-acid (40 mg, 0.226 mmol) in DMF at room temperature was added triethylamine (0.157 mL, 1.12 mmol) and then palmitic acid NHS ester (90 mg, 0.248 mmol). The mixture was stirred at room temperature for 15 min and then COMU (97 mg, 0.225 mmol) and 2,3,5,6-tetrafluorophenol (37 mg, 0.225 mmol) were added and the mixture was stirred at room temperature for 30 min. The reaction was diluted with EtOAc and washed consecutively with brine, dried over MgSO₄, filtered, and concentrated under reduced pressure to give crude material that was purified on a 4 g silica gel column (EtOAc/hexanes 0-50%) to give LP-208-p (61 mg) as a white wax in 48% yield. LC-MS: calculated [M+H] 564.33, found 564.83.

### Synthesis of LP-211-p

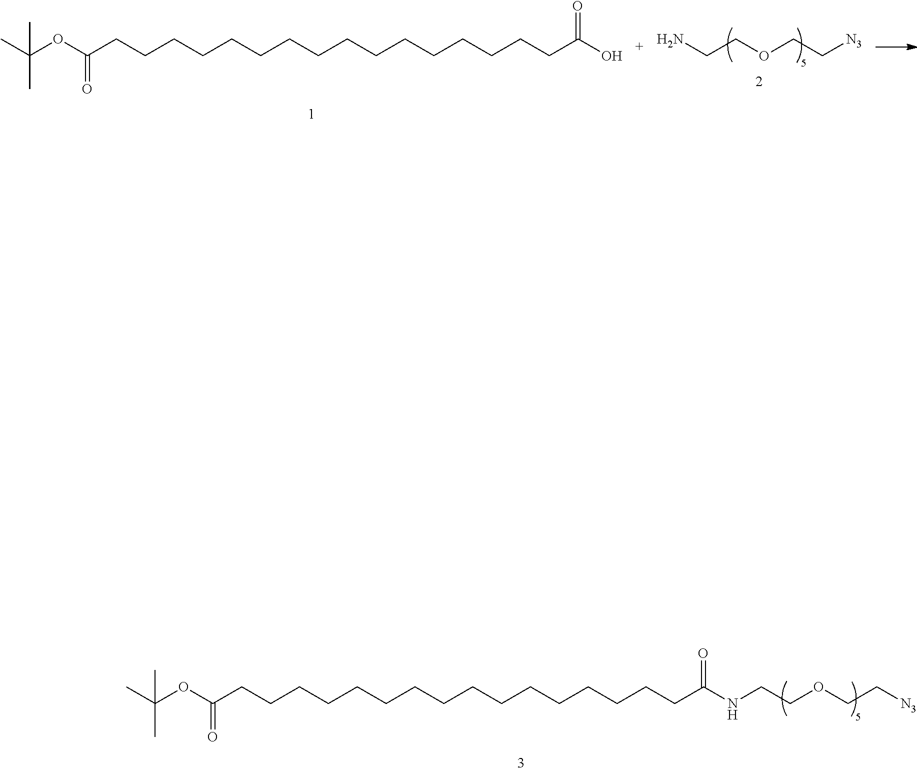

Compound 1 (Asta Tech® #64704, 0.75 g) was dissolved in 16 mL of DMF, then TBTU (0.71 g) and DIPEA (0.89 mL) were added. The mixture was stirred for 10 minutes, then Compound 2 (0.68 g) in DMF was added. The reaction was allowed to proceed for one hour, then the mixture was diluted with 120 mL EtOAc and washed with 5% Citric Acid, water (3×20 mL), NaCl (1×15 mL) dried with Na₂SO₄, filtered, and concentrated on rotary evaporator and high vacuum. The product was purified on silica gel column, MeOH/DCM (0-5%) over 25 minutes. Yield 927 mg. LC-MS: calculated [M+H] 658.92, found 659.90.

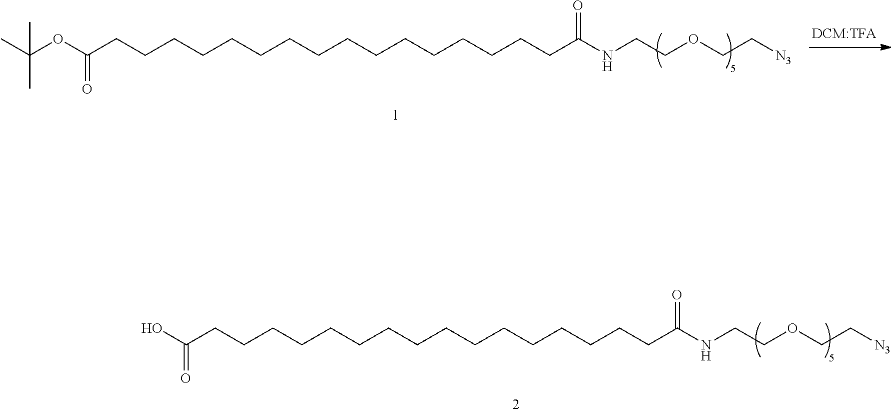

Compound 1 (0.92 g) was dissolved in a mixture of 1:1 DCM:TFA (6 mL) The reaction was stirred for 1 hour. The compound was concentrated in vacuo. Yield: 851 mg. LC-MS: calculated [M+H] 602.81, found 603.84.

### Synthesis of LP-232-p

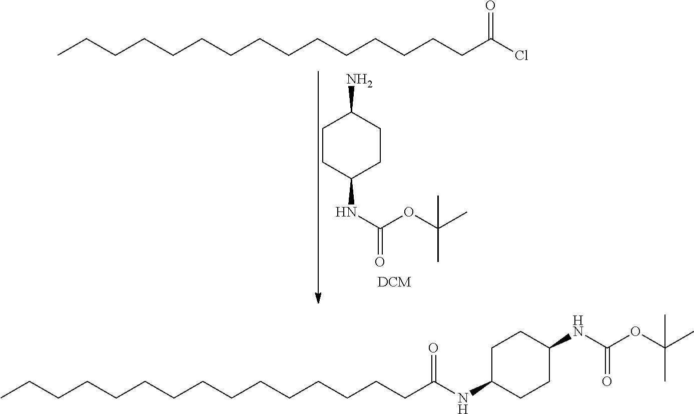

Palmitoyl chloride (100 mg) was stirred in a solution of cis-4-(boc-amino)cyclohexylamine (0.0819 g) in 5 mL DCM. After stirring the suspension overnight, water was added and the organics were extracted using DCM and dried over Na₂SO₄. After filtration, the solvent was concentrated to dryness and the crude product was purified by flash chromatography (Hexanes to EtOAc). Yield 52 mg, 31%.

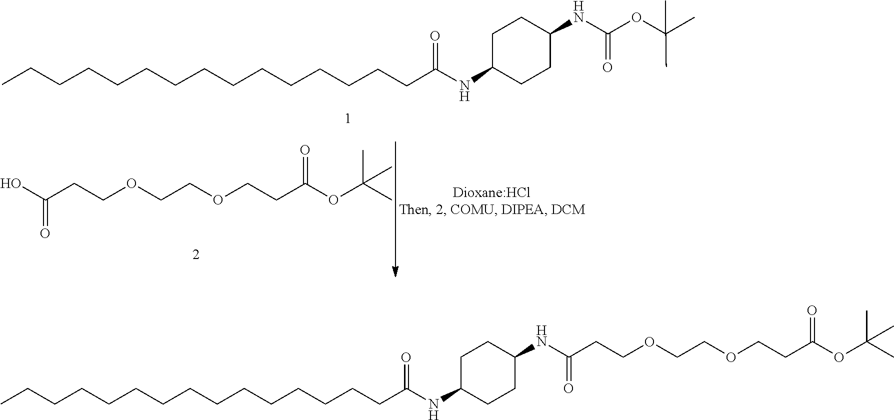

To 1 (0.0520 g) was added 2 mL Dioxane:HCl (4N) until boc deprotection was complete. After removing solvent in vacuo, to the crude residue was added a solution of 2 (0.0316 g), DIPEA (0.0445 g) and COMU (0.0620 g) in 5 mL DCM. After stirring the suspension overnight, water was added and the organics were extracted using DCM and dried over Na₂SO₄. After filtration, the solvent was concentrated to dryness and the crude product was purified by column (MeOH/DCM 0-20%). Yield 45 mg, 65%.

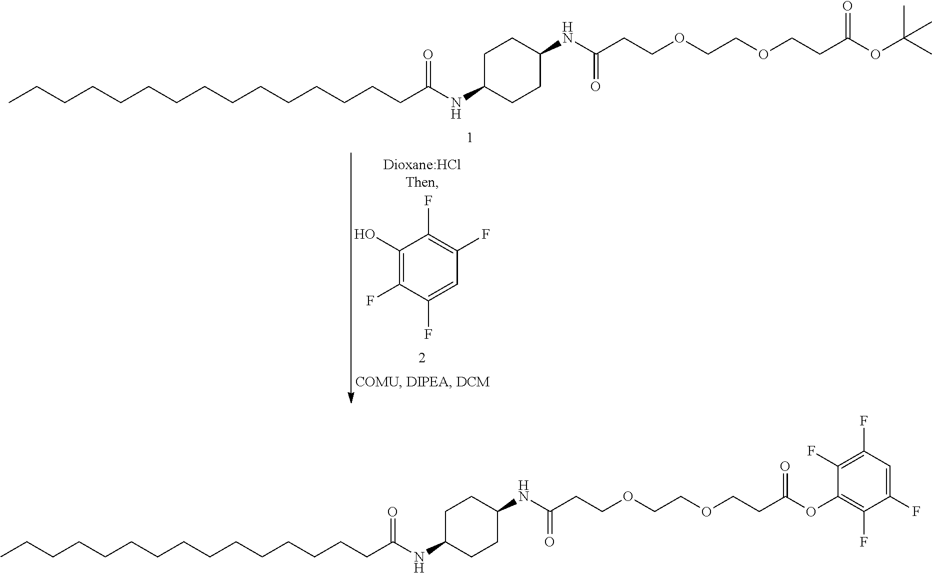

To 1 (0.0449 g) was added 2 mL Dioxane:HCl (4N) until OtBu deprotection was complete. After removing solvent in vacuo, to the residue was stirred in a solution of 2 (0.0217 g), DIPEA (0.039 mL) and COMU (0.0425 g) in 5 mL DCM. After stirring the suspension overnight, water was added and the organics were extracted using DCM and dried over Na₂SO₄. After filtration, the solvent was concentrated to dryness and the crude product was purified by column (MeOH/DCM, 0-20%). Yield 30 mg, 58%.

### Synthesis of LP-242-p

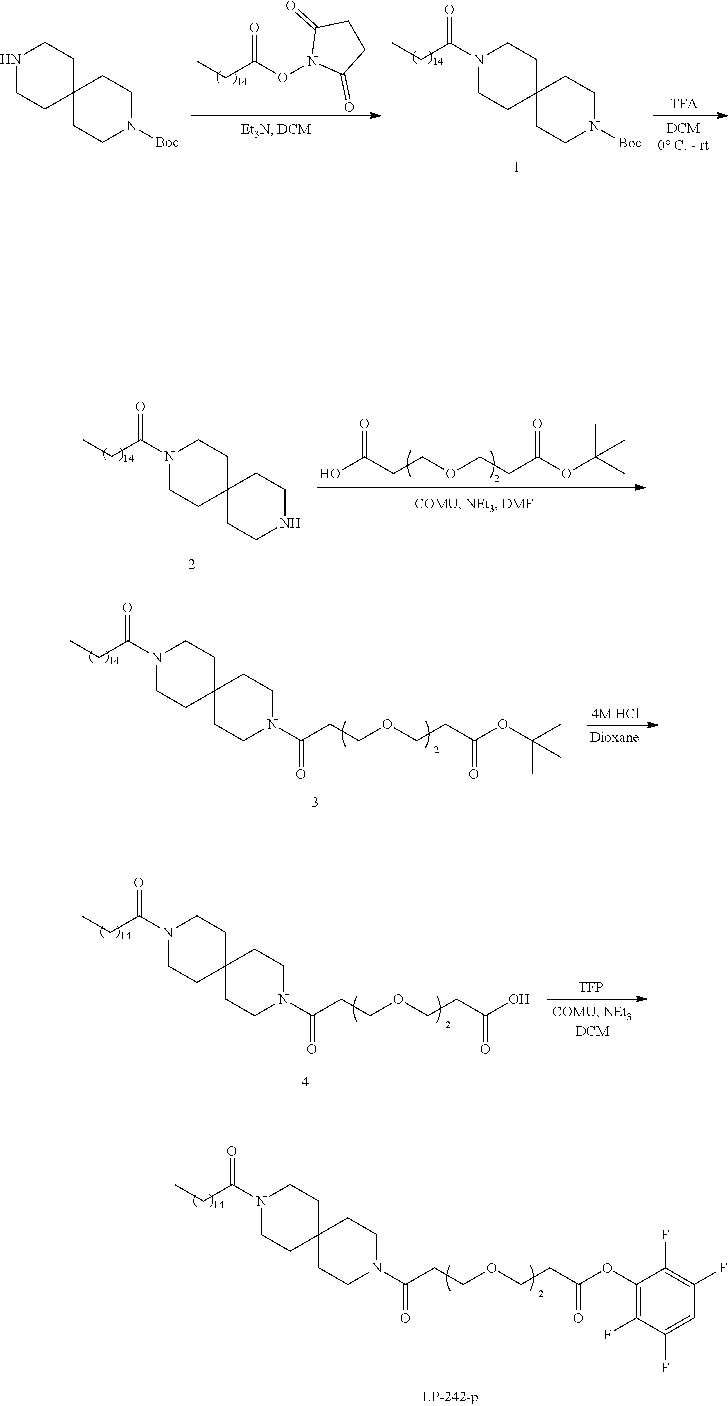

tert-Butyl 3,9-Diazaspiro[5.5]undecane-3-carboxylate (300 mg, 1.17 mmol) was dissolved in DCM and triethylamine (0.82 mL, 5.89 mmol) was added. Palmitic acid NHS ester (416 mg, 0.46 mL, 1.17 mmol) was then added to the solution and the reaction was stirred for 60 min. LC-MS indicated reaction completion. The reaction was quenched with 2 M HCl (20 mL) and the layers separated. The organic layer was washed with 1 M HCl (2×20 mL), H₂O (3×20 mL), sat. NaCl (20 mL), dried over Na₂SO₄ and concentrated to yield a white solid that was purified by silica gel chromatography (MeOH/DCM 0-4%) to yield 1 (484 mg) as a white solid in 83% yield. Intermediate 1 (484 mg, 0.982 mmol) was dissolved in DCM, cooled to 0° C., and TFA (2 mL, 26 mmol) was added. The reaction was allowed to warm to room temperature after the addition of TFA and stirred for 60 min. LC-MS indicated reaction completion. The reaction was quenched with sat. aq. NaHCO₃ until the solution remained basic and the mixture was then extracted with DCM (3×15 mL), washed with brine (2×30 mL), dried over Na₂SO₄, and concentrated to give 2 (385 mg) an off-white solid that was used without further purification. Acid-PEG₂-tert-butyl ester (252 mg, 0.962 mmol) was dissolved in DCM followed by the addition of triethylamine (3.4 mL, 24.5 mmol) and COMU (412 mg, 0.962 mmol). The reaction was allowed to stir for 3 min before the addition of intermediate 2 (385 mg, 0982 mmol) and the reaction was stirred for 30 min. LC-MS confirmed reaction completion. The reaction was quenched with 2 M HCl (20 mL) and the layers separated. The organic layer was washed with H₂O (3×20 mL), sat. NaCl (20 mL), dried over Na₂SO₄ and concentrated to yield a white crude solid that was purified by silica gel chromatography (MeOH/DCM, 0-10%) to yield 3 (585 mg) as a white solid in 95% yield. LC/MS (ESI⁺) calculated m/z 636.96, found 637.46 (M+H⁺).

Intermediate 3 (585 mg, 0.918 mmol) was dissolved in 4 M HCl in dioxane (15 mL) and the reaction was stirred until no starting material was observed by LC-MS. Nitrogen was bubbled through the reaction to remove the majority of the HCl and the solvent was then removed under reduced pressure and the solid was dried on vacuum to give 4 (538 mg) as white solid that was used without purification. Intermediate 4 (538 mg, 0.926 mmol) and COMU (436 mg, 1.01 mmol) were dissolved in DCM and triethylamine (1.3 mL, 9.26 mmol) was added. The reaction was stirred for 3 min and then 2,3,5,6-tetrafluorophenol (169 mg, 1.01 mmol) was added. After 20 minutes LC-MS indicated reaction completion. The reaction mixture was concentrated and loaded directly onto a silica gel column and purified by silica gel chromatography MeOH/DCM, 0-20%) to give LP-242-p (372 mg) as an off-white solid in 50% yield. LC/MS (ESI⁺) calculated m/z 728.91, found 729.63 (M+H⁺)

### Synthesis of LP-243-p

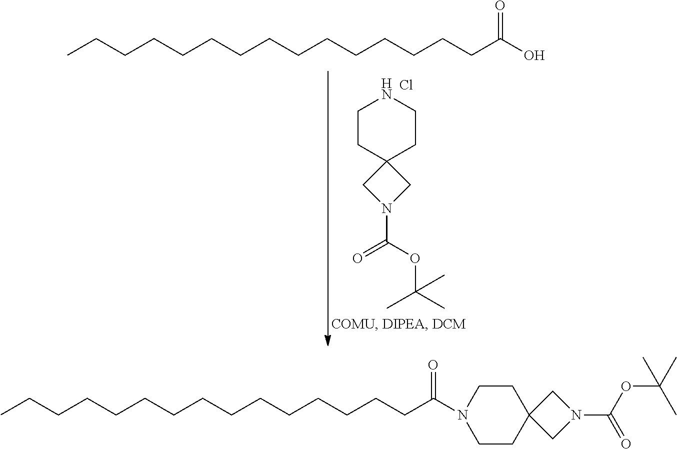 [SMILES: CCCCCCCCCCCCCCCC(=O)O ; Cl ; CC(C)(C)OC(=O)N1CC2(CCNCC2)C1 ; CCCCCCCCCCCCCCCC(=O)N1CCC2(CC1)CN(C(=O)OC(C)(C)C)C2]

To palmitic acid (0.100 g) was added a solution of tBu-3,9diazaspiro[5,5]undecane-3-carboxylate HCl (0.0732 g), COMU (0.166 g), and DIPEA (0.161 mL) in 5 mL DCM. The suspension was stirred overnight at 40° C. (oil bath temp). Water was added and the organics were extracted using DCM and dried over Na₂SO₄. After filtration, the solvent was concentrated to dryness and the crude product was purified by flash chromatography (MeOH/DCM, 0-5%).

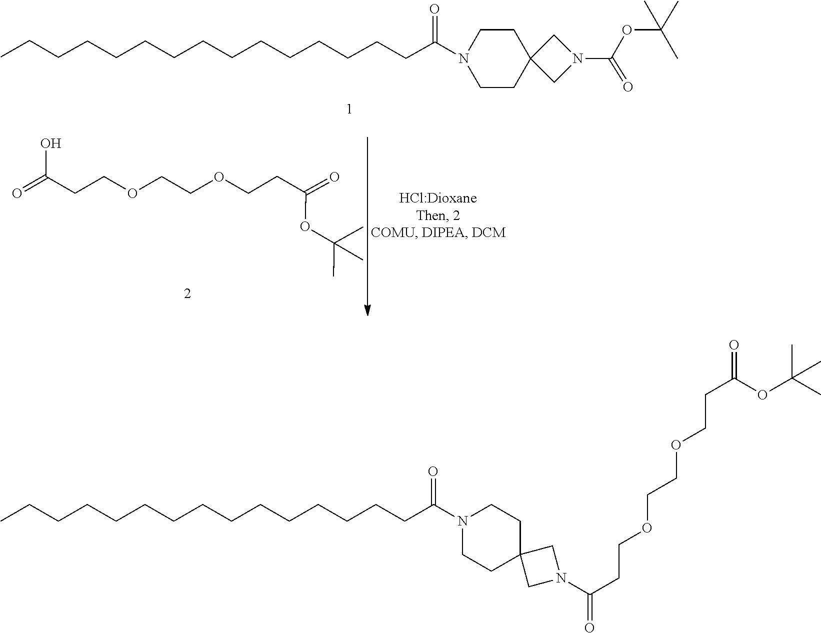

1 (0.0200 g) was treated with 4 M HCl:Dioxane and stirred for 1 h. The reaction was dried in vacuo. To the crude product was added a solution of 2 (0.0119 g), COMU (0.0232 g), and DIPEA (0.022 mL). After stirring the suspension overnight, water was added and the organics were extracted using DCM and dried over Na₂SO₄. After filtration, the solvent was concentrated to dryness and the crude product was purified by flash chromatography.

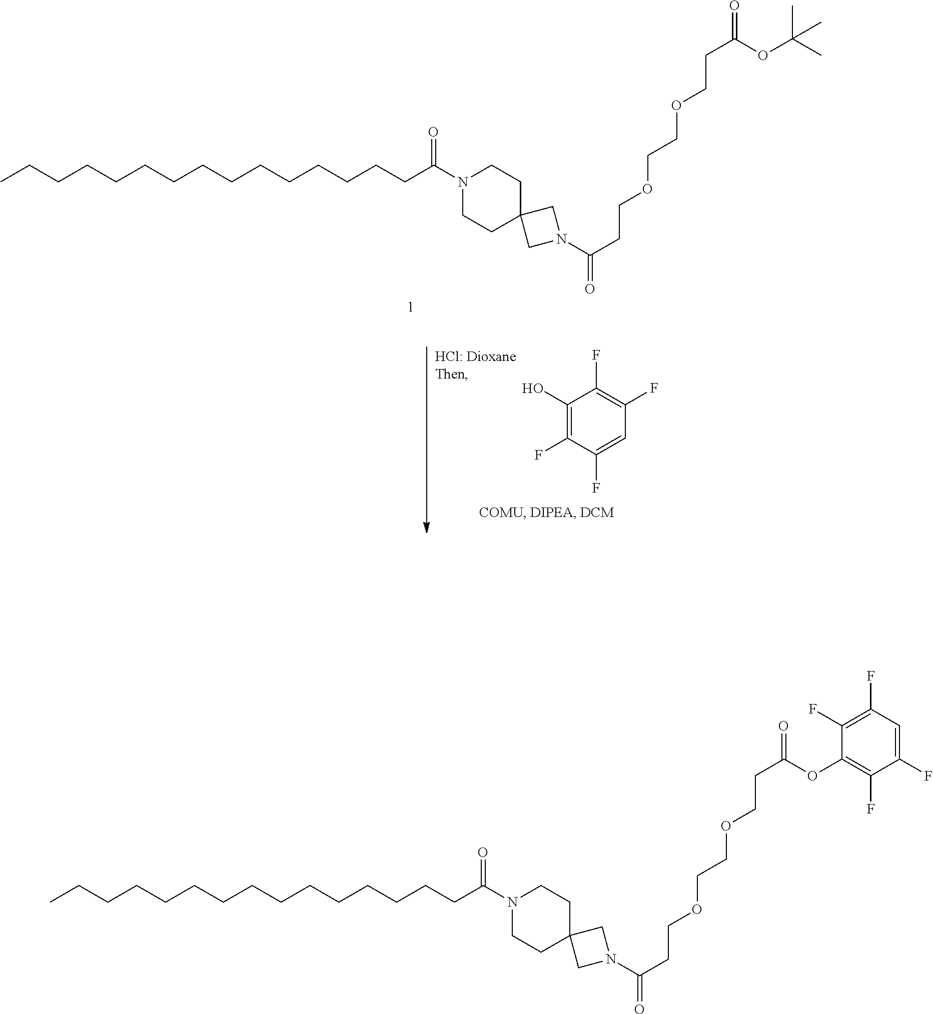

To 1 (0.121 g) was added 2 mL Dioxane:HCl (4 N) until otBu deprotection was complete. After removing the solvent in vacuo, crude 1 was stirred in a solution of tetrafluorophenol (0.0363 g), DIPEA (0.104 mL) and COMU (0.112 g) in 5 mL DCM. After stirring the suspension overnight, water was added and the organics were extracted using DCM and dried over Na₂SO₄. After filtration, the solvent was concentrated to dryness and the crude product was purified by flash chromatography.

### Synthesis of LP-244-p

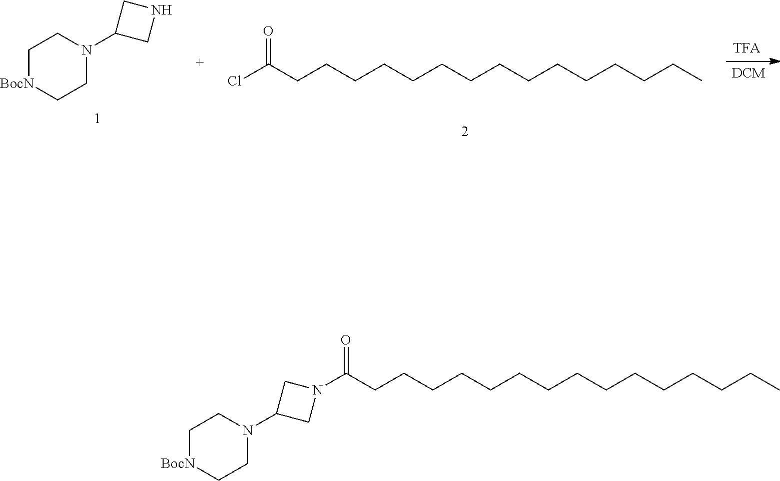 [SMILES: CC(C)(C)OC(=O)N1CCN(C2CNC2)CC1 ; CCCCCCCCCCCCCCCC(=O)Cl ; CCCCCCCCCCCCCCCC(=O)N1CC(N2CCN(C(=O)OC(C)(C)C)CC2)C1]

To the mixture of 1 (0.100 g) and 2 (0.251 mL) in DCM was added TEA (0.174 mL) at room temperature. The reaction mixture was stirred at room temperature for 2 h. The reaction was washed with water, dried over Na₂SO₄, filtered, and concentrated under reduced pressure to give crude material that was purified with flash chromatography (EtOAc/hexanes). Product was a white solid, 147 mg, 74%. LC-MS: calculated [M+H] 480.42, found 480.85.

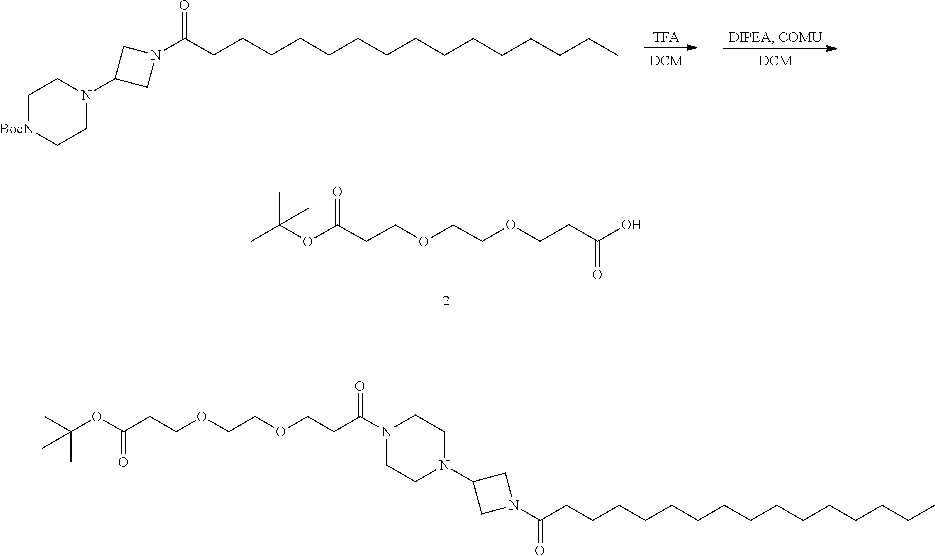

To the solution of 1 (0.147 g) in DCM was added TFA (1 mL) at room temperature. The reaction mixture was stirred at room temperature for 0.5 h. The solvent was removed in vacuo, then the residue was placed under high vacuum for 2 h. The residue was dissolved in 3 mL DMF, then 2 (0.0800 g), DIPEA (0.0160 mL) and COMU (0.197 g) were added at room temperature. The mixture was stirred at room temperature for 2 h. The solvent was removed in vacuo. Purification was on a 12 g column. DCM to 20% MeOH in DCM as gradient was used to purify. Product was a clear oil, 173 mg, 91%. LC-MS: calculated [M+H] 624.50, found 625.33.

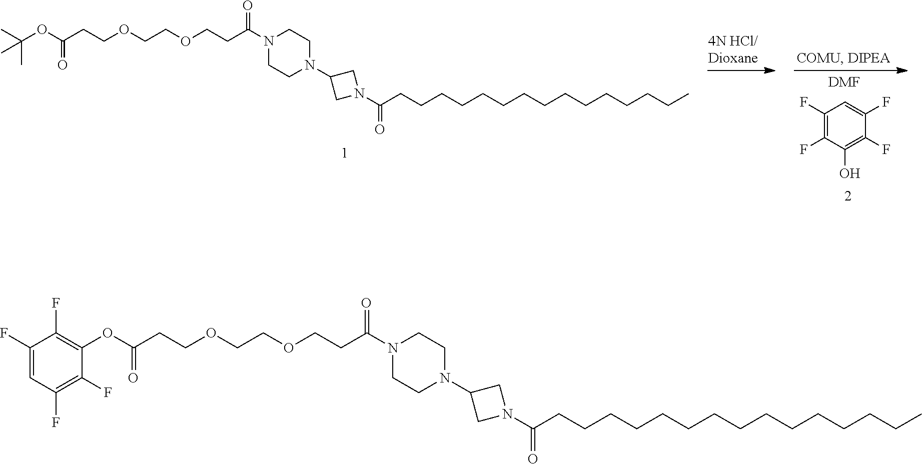

A solution of 1 (0.173 g) in 4N HCl/Dioxane (6 mL) was stirred at room temperature overnight. After removing the solvent in vacuo, the residue was placed under high vacuum for 3 h. The residue was dissolved in 3 mL DMF, then, DIPEA (0.145 mL), COMU (0.356 g) and 2 (0.0920 g) were added. The mixture was stirred at room temperature for 2 h. After removing solvent in vacuo, the residue was loaded on a 12 g column. DCM to 20% MeOH in DCM as gradient was used to purify. Product was a light yellow solid, 68 mg, 37%. LC-MS: calculated [M+H] 716.43, found 717.23.

### Synthesis of LP-245-p

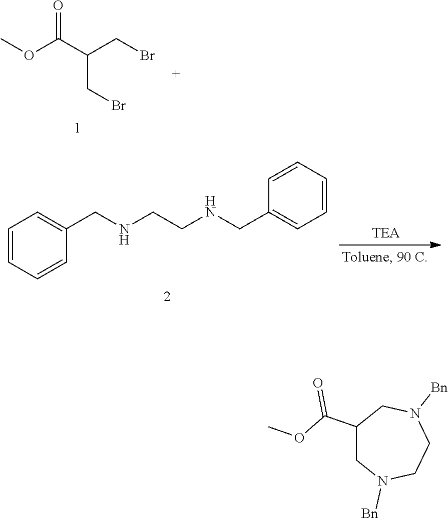

To a mixture of 1 (2.08 g) and 2 (1.98 g) in 50 mL toluene was added TEA at room temperature. The reaction mixture was stirred at 90° C. overnight. After cooling to room temperature, the reaction was diluted with EtOAc, washed with H₂O (×2), brine, dried over Na₂SO₄, filtered, and concentrated under reduced pressure. Purification was on a 40 g column (EtOAc/hexanes, 0-30%). Product was a light yellow oil, Yield 1388 mg, 51%. LC-MS: calculated [M+H] 339.21, found 339.62.

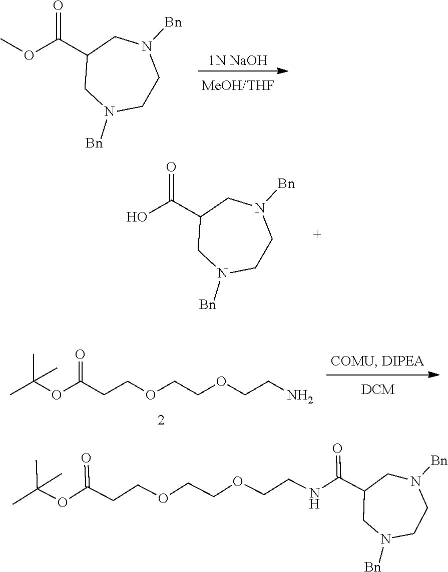

To a mixture of 1 (0.241 g) in MeOH/THF (4 mL/4 mL) was added 1N NaOH (6 mL) at room temperature. The reaction mixture was stirred at 60° C. for 1 h. After removing the organic solvent in vacuo, 1N HCl was added to adjust the mixture to pH˜1. Then NaHCO₃ was added to adjust pH between 7˜8. The product was extracted with DCM, dried over Na₂SO₄, filtered, concentrated, and placed under vacuum pump. The residue was dissolved in DCM, then DIPEA (0.248 mL), COMU (0.336 g) and 2 (0.166 g) were added. The reaction mixture was stirred at room temperature for 2 h. The reaction mixture was washed with 1N HCl, NaHCO₃ and brine, dried over Na₂SO₄, filtered, and concentrated under reduced pressure. Purification was on a 12 g column (EtOAc/hexanes). Product was a brown oil, 285 mg, 74%. LC-MS: calculated [M+H] 540.34, found 541.07.

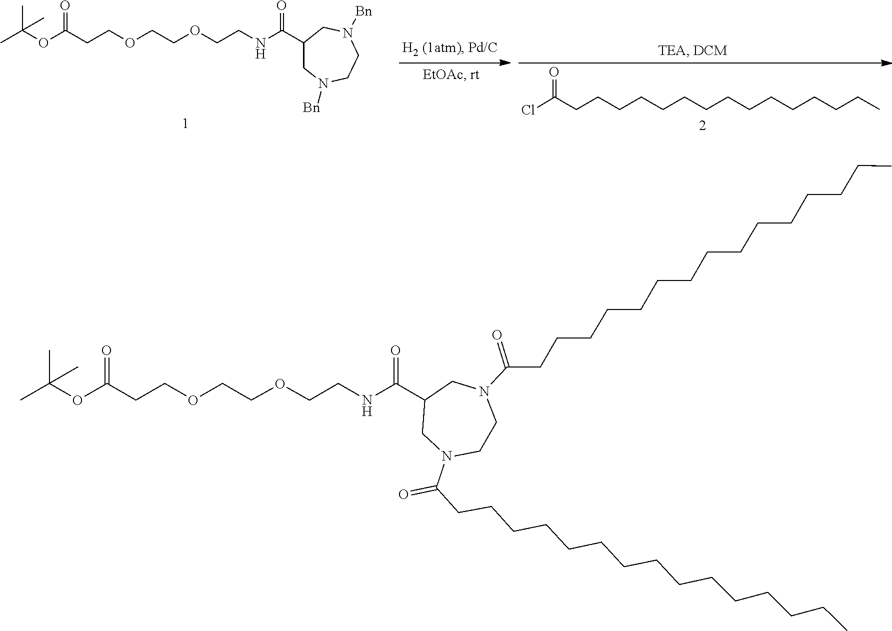 [SMILES: CC(C)(C)OC(=O)CCOCCOCCNC(=O)C1CN(Cc2ccccc2)CCN(Cc2ccccc2)C1 ; CCCCCCCCCCCCCCCC(=O)Cl ; CCCCCCCCCCCCCCCC(=O)N1CCN(C(=O)CCCCCCCCCCCCCCC)CC(C(=O)NCCOCCOCCC(=O)OC(C)(C)C)C1]

A mixture of 1 (0.0740 g) and Pd/C in EtOAc was degassed with N₂ and then charged with H₂ (1 atm) at room temperature. The reaction mixture was stirred at room temperature for 4 h. The reaction mixture was filtered with Celite®. After removing EtOAc in vacuo, the residue was further dried under high vacuum for 1 h. The residue was dissolved in 3 mL DCM, 2 (0.166 mL) and TEA (0.115 mL) were added at room temperature. The mixture was stirred at room temperature for 2 h. The reaction was washed with H₂O, brine, dried over Na₂SO₄, filtered, and concentrated under reduced pressure to give crude material that was purified with a 12 g silica gel column (MeOH/DCM, 0-20%). Product was a clear oil, 43 mg, 37%. LC-MS: calculated [M+H] 836.71, found 837.68.

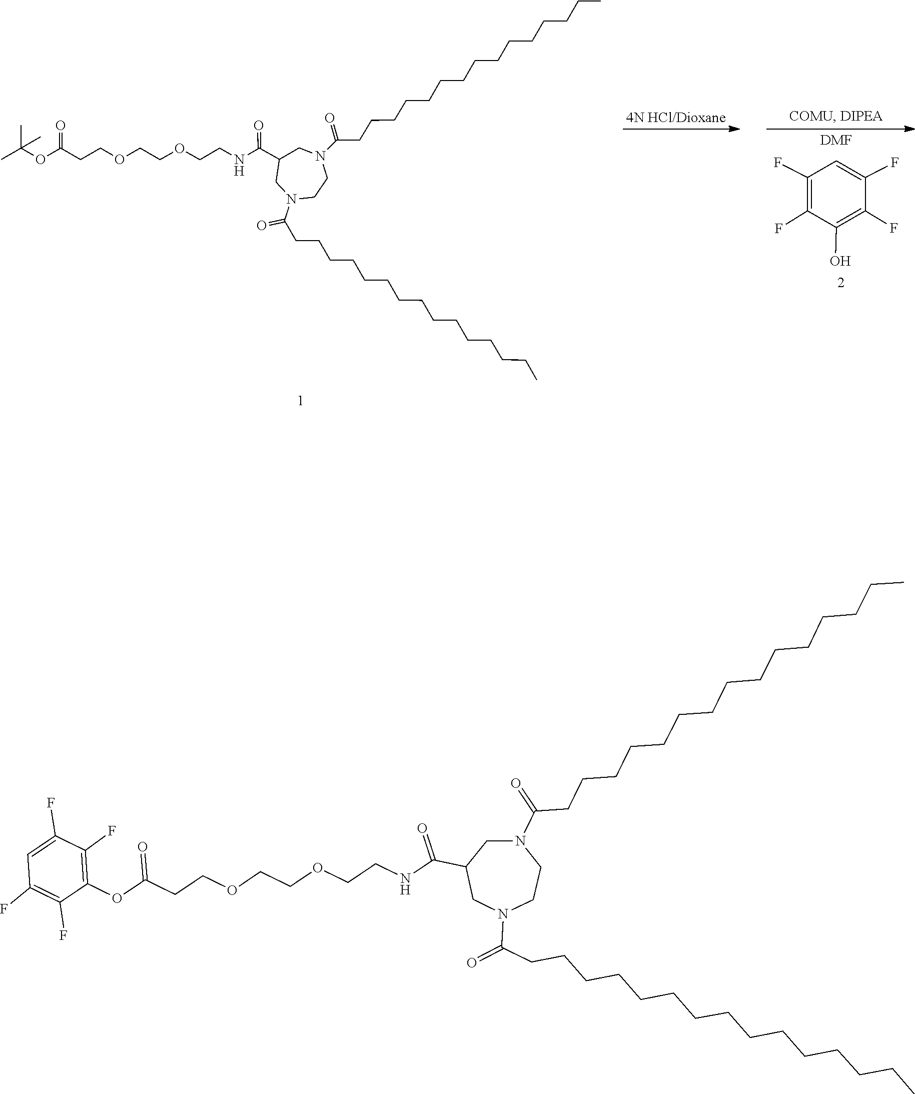

A solution of 1 (0.0430 g) in 4N HCl/Dioxane (3 mL) was stirred at room temperature overnight. After removing solvent in vacuo, the residue was placed under high vacuum for 3 h. The residue was dissolved in 3 mL DMF, then, DIPEA (0.027 g), COMU (0.0660 g) and 2 (0.017 g) were added. The mixture was stirred at room temperature for 2 h. After removing solvent in vacuo, the residue was directly loaded onto a 4 g column and purified with flash chromatography (MeOH/DCM, 0-20%). Product was a light yellow oil, 34 mg, 37%. LC-MS: calculated [M+H] 928.64, found 929.59.

### Synthesis of LP-249p

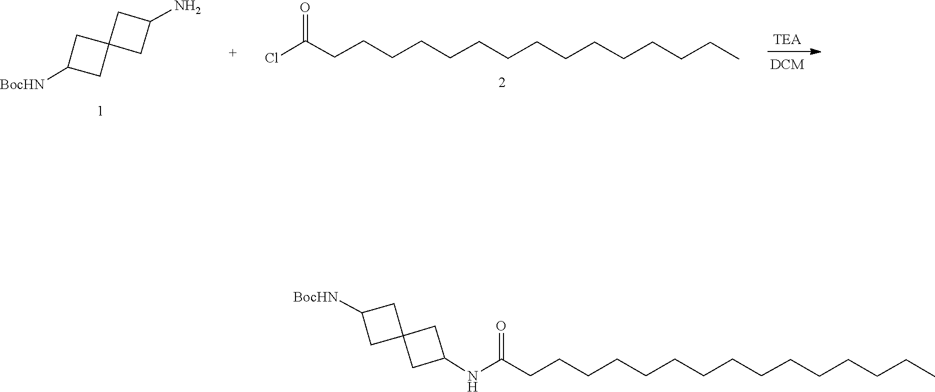 [SMILES: CC(C)(C)OC(=O)NC1CC2(CC(N)C2)C1 ; CCCCCCCCCCCCCCCC(=O)Cl ; CCCCCCCCCCCCCCCC(=O)NC1CC2(C1)CC(NC(=O)OC(C)(C)C)C2]

To a mixture of 1 (0.0600 g) and 2 (0.161 mL) in 4 mL DCM was added TEA (0.111 mL) at room temperature. The reaction mixture was stirred at room temperature for 2 h. The reaction was washed with H₂O, dried over Na₂SO₄, filtered, and concentrated under reduced pressure to give crude material that was purified with a 4 g silica gel column (EtOAc/hexanes). Product was a white solid, 74 mg, 60%. LC-MS: calculated [M+H] 465.41, found 465.91.

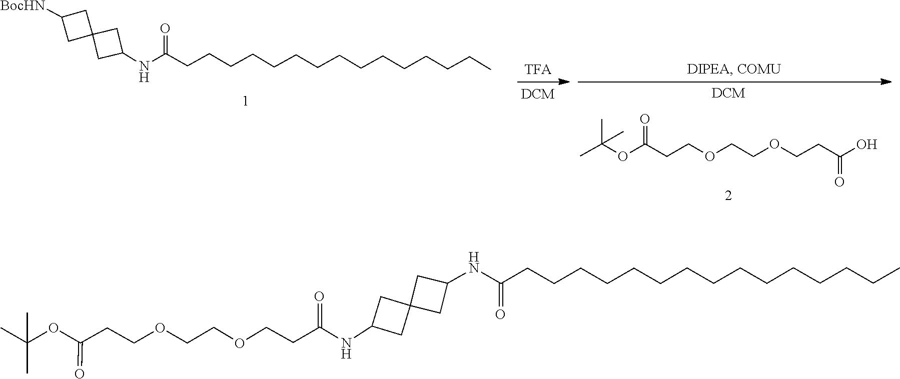 [SMILES: CCCCCCCCCCCCCCCC(=O)NC1CC2(C1)CC(NC(=O)OC(C)(C)C)C2 ; CC(C)(C)OC(=O)CCOCCOCCC(=O)O ; CCCCCCCCCCCCCCCC(=O)NC1CC2(C1)CC(NC(=O)CCOCCOCCC(=O)OC(C)(C)C)C2]

To a solution of 1 (0.0740 g) in DCM was added TFA (50% in DCM) at room temperature. The reaction mixture was stirred at room temperature for 0.5 h. The solvent was removed in vacuo, then the residue was under high vacuum for 2 h. The residue was dissolved in DMF, then 2 (0.0420 g), DIPEA (0.084 mL) and COMU (0.102 g) were added at room temperature. The mixture was stirred at room temperature for 2 h. The solvent was removed in vacuo and the crude reaction was dry loaded onto a 12 g column and purified with flash chromatography (MeOH/DCM, 0-20%). Product was a white solid, 56 mg, 58%. LC-MS: calculated [M+H] 609.48, found 610.29.

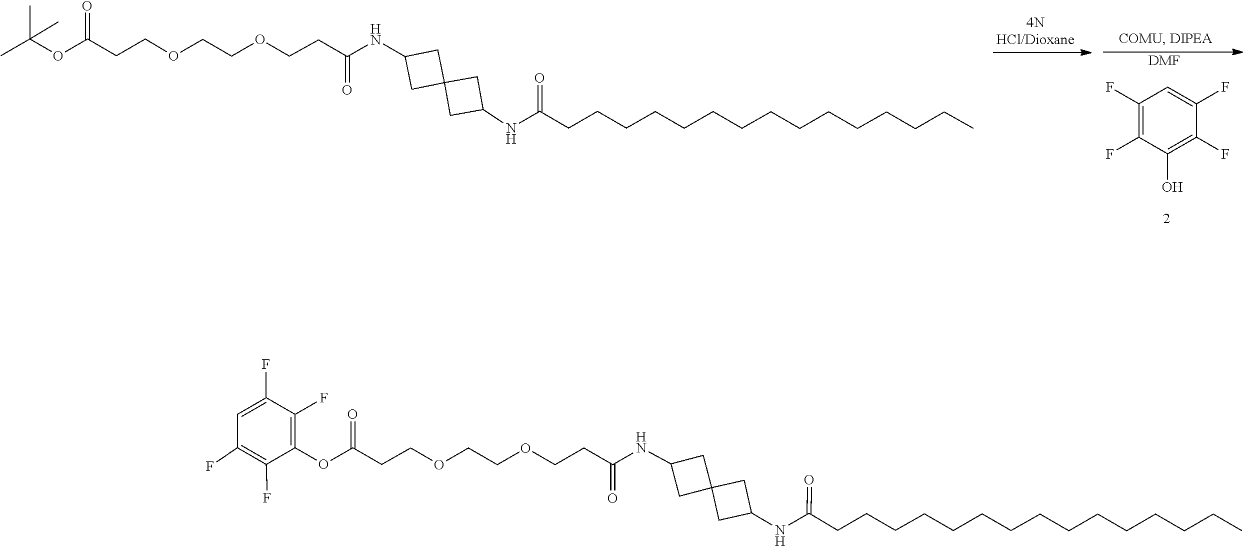

The solution of 1 (0.0560 g) in 4N HCl/Dioxane (3 mL) was stirred at room temperature overnight. After removing solvent in vacuo, the residue was placed under high vacuum for 3 h. The residue was dissolved in 2 mL DMF, then, DIPEA (0.048 mL), COMU (0.118 g) and 2 (0.031 g) were added. The mixture was stirred at room temperature for 2 h. After removing solvent in vacuo, the residue was dry loaded onto a 4 g column and purified with flash chromatography (MeOH/DCM, 0-20%). Product was an off-white solid, 16 mg, 25%. LC-MS: calculated [M+H] 701.42, found 702.20.

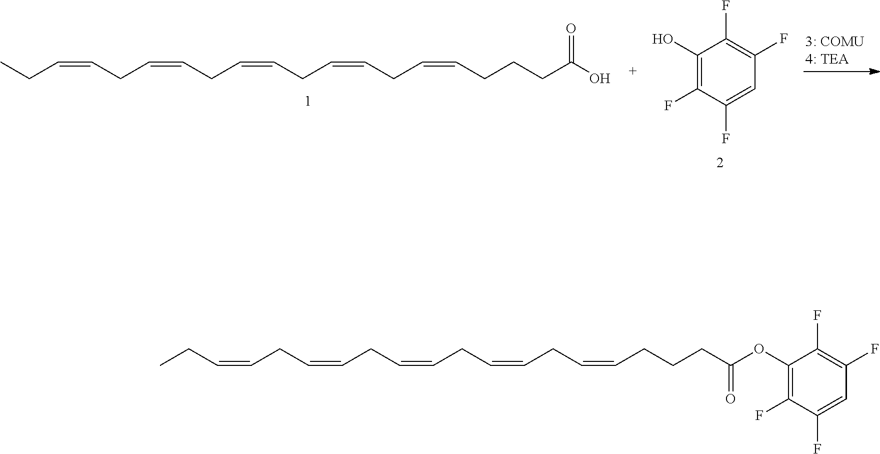 [SMILES: CC/C=C\C/C=C\C/C=C\C/C=C\C/C=C\CCCC(=O)O ; Oc1c(F)c(F)cc(F)c1F ; CC/C=C\C/C=C\C/C=C\C/C=C\C/C=C\CCCC(=O)Oc1c(F)c(F)cc(F)c1F]

To a solution of EPA 1 (60.5 mg, 0.200 mmol, 1 eqv.) and 2 (36.5 mg, 0.220 mmol, 1.10 eqv.) in 20 mL DCM was added COMU (94.2 mg, 0.220 mmol, 1.10 eqv.) and then TEA (0.084 mL, 0.600 mmol, 3.0 eqv.) under ambient conditions. The reaction was stirred until full conversion was observed by LC-MS. The reaction mixture was washed with 1N HCl, brine, dried over Na₂SO₄, filtered, and concentrated. The reaction mixture was purified by CombiFlash® using silica gel as the stationary phase with a gradient of EtOAc/hexanes, 0-50%. 69 mg product was obtained (76% yield).

### Synthesis of LP-295-p

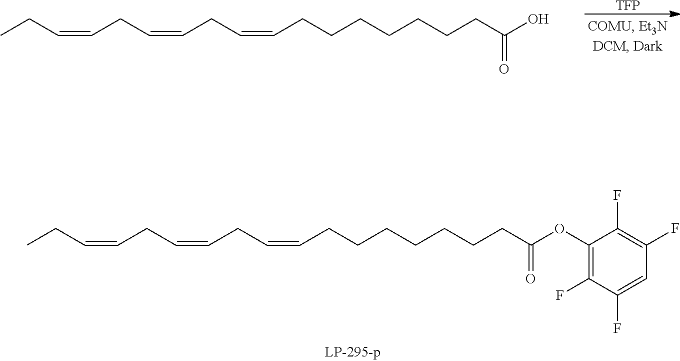 [SMILES: "TFP" ; CC/C=C\C/C=C\C/C=C\CCCCCCCC(=O)O ; CC/C=C\C/C=C\C/C=C\CCCCCCCC(=O)Oc1c(F)c(F)cc(F)c1F]

To a 40 mL vial with stir bar and under N2 was added α-linolenic acid (100 mg, 0.359 mmol), 2,3,5,6-tetrafluorophenol (72 mg, 0.431 mmol), DCM (4 mL), and triethylamine (0.15 mL, 1.08 mmol). COMU 185 mg, 0.431 mmol) was added, the reaction was wrapped with foil, and stirred at room temperature for 2 h. TLC (kMnO₄ stain) confirmed reaction completion. The reaction was diluted with DCM and washed with H₂O (×3), saturated NaHCO₃ (×3), brine, dried over MgSO₄, filtered, and concentrated under reduced pressure (shielding product from light) to give LP-295-p (152 mg) as a crude red oil that was used without further purification. LC/MS (ESI⁺) calculated m/z 426.22 (M), found 427.48 (M+H⁺). ¹H NMR (400 MHz, CDCl₃) δ=7.02-6.94 (m, 1H), 5.44-5.28 (m, 6H), 2.80 (app. t., 4H), 2.66 (t, 2H), 2.10-2.02 (m, 4H), 1.77 (q, 2H), 1.48-1.31 (br. m., 8H), 0.972 (t, 3H).

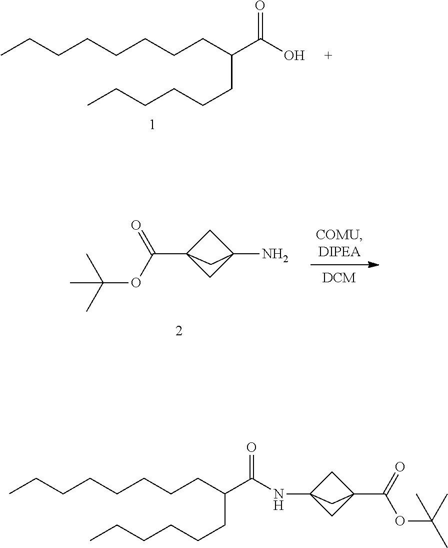 [SMILES: CCCCCCCCC(CCCCCC)C(=O)O ; CC(C)(C)OC(=O)C12CC(N)(C1)C2 ; CCCCCCCCC(CCCCCC)C(=O)NC12CC(C(=O)OC(C)(C)C)(C1)C2]

To the solution of 1 in DCM was added DIPEA (0.057 mL), COMU (0.077 g) and 2 (0.0300 g) at room temperature. After stirring at room temperature for 2 h, the reaction was quenched with 0.1N HCl. The organic layer was washed with brine, dried over Na₂SO₄, filtered, and concentrated under reduced pressure. The residue was loaded onto a 4 g column and purified with flash chromatography (EtOAc/hexanes, 0-50%). Product was a white solid, 46 mg, 44%. LC-MS: calculated [M+H] 422.36, found 422.61.

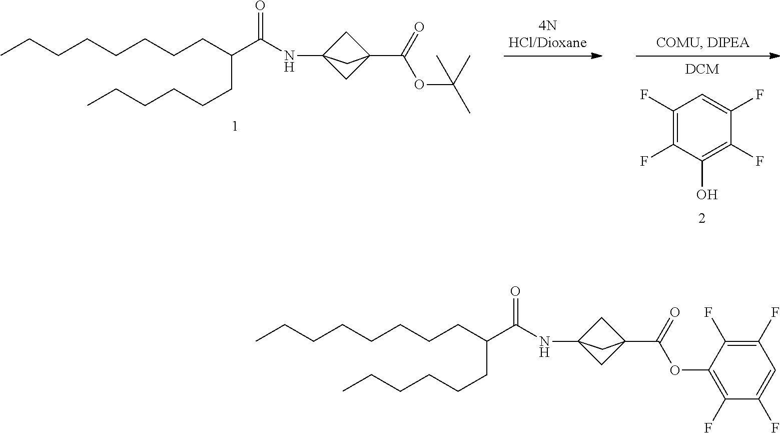

The solution of 1 (0.046 g) in 4N HCl/Dioxane (2 mL) was stirred at room temperature overnight. After removing the solvent in vacuo, the residue was placed under high vacuum for 3 h. Then the residue was dissolved in DCM at room temperature, then COMU (0.0700 g), DIPEA (0.038 mL) and 2 (0.036 g) were added at room temperature. After stirring at room temperature for 2 h, the solvent was removed in vacuo. The residue was loaded onto a 4 g silica column and purified with flash chromatography (EtOAc/hexanes, 0-50%). Product was a white solid, 21 mg, 38%. LC-MS: calculated [M+H] 514.29, found 514.61.

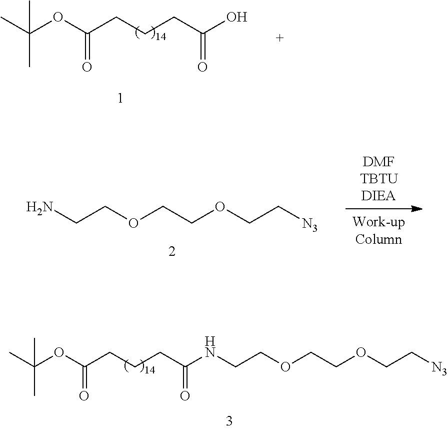

Compound 1 (Asta Tech® #64704, 250 mg) was dissolved in 6 mL of DMF. Then TBTU (238 mg) and DIPEA (0.48 mL) were added. The mixture was stirred for 10 minutes, then compound 2 (129 mg) in DMF was added. After stirring for 1 hour, the reaction mixture was diluted with 75 mL EtOAc and washed with 3% citric acid (3×8 mL), H₂O (2×8 mL), NaCl (1×8 mL) dried with Na₂SO₄, filtered and concentrated on rotary evaporator and high vacuum. The product was purified by dry loading in 5 mL silica onto 12 G RediSep Gold Rf column (MeOH/DCM, 0-5% over 30 min.) Yield 287 mg. LC-MS: calculated [M+H] 526.76, found 527.39.

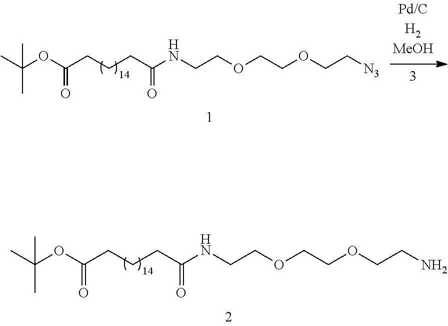

Compound 1 (287 mg) was dissolved in 10 mL MeOH. Then 104 mg of Pd/C was added. The vessel was purged with N2 and H2 was introduced by balloon. The reaction was stirred overnight. The mixture was filtered over Celite® and the pad was washed with ethanol. The product was concentrated on rotary evaporator and high vacuum. Yield 254 mg. LC-MS: calculated [M+H] 500.77, found 501.46.

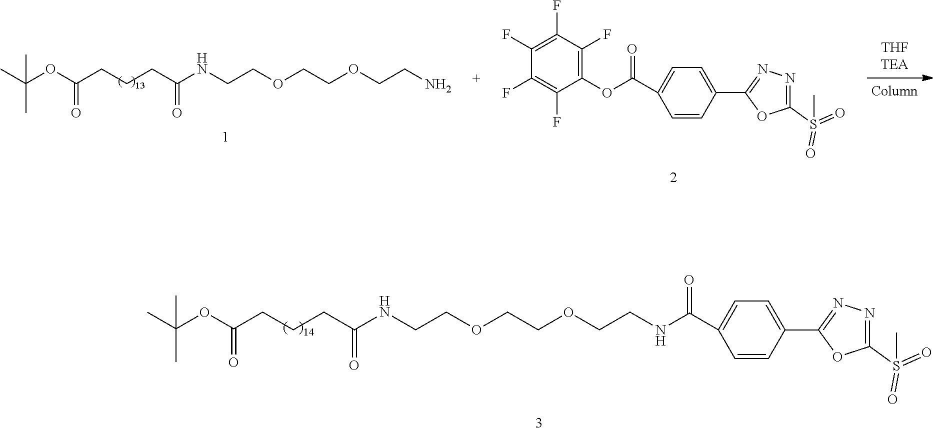

Compound 1 (20 mg) was dissolved in 1.2 mL THF. Then TEA (0.033 mL) and compound 2 (22 mg) were added. The reaction was stirred for 1 hour. The mixture was concentrated in vacuo then dry loaded with celite onto a silica gel column and purified with flash chromatography dry MeOH/DCM 0-10% over 25 minutes. Yield: 19 mg. LC-MS: calculated [M+H] 750.99, found 751.85.

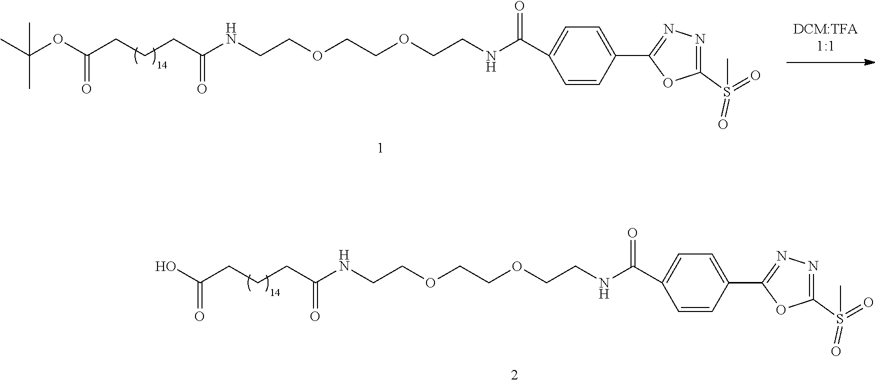

Compound 1 (19 mg) was dissolved in 1:1 DCM:TFA (1.0 mL). The reaction was stirred for 1 hour. A very small amount of toluene was added and the reaction was concentrated in 28° C. water bath and concentrated in rotary evaporator and high vacuum. Yield: 16 mg. LC-MS: calculated [M+H] 694.89, found 695.99.

### Synthesis of LP-361-p

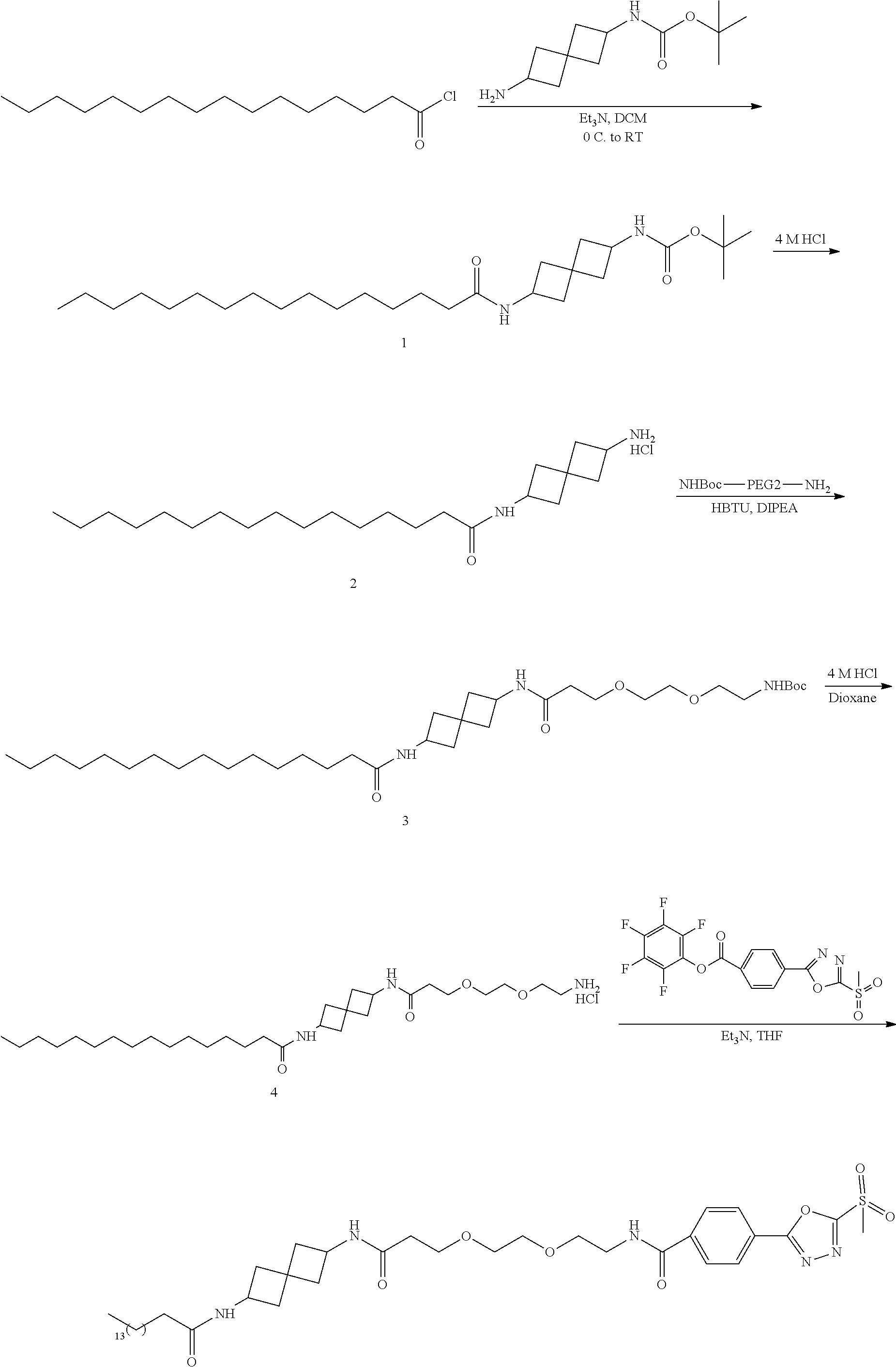

To a 40 mL vial with stir bar and under N2 was added N-(2-aminospiro[3.3]hept-6-yl)carbamic acid tert-butyl ester (350 mg, 1.55 mmol), DCM (10 mL), and triethylamine (0.65 mL, 4.65 mmol). The reaction was stirred and cooled in an ice bath for 10 min and then palmitoyl chloride (0.51 mL, 1.7 mmol) was added dropwise over 30 seconds. A white precipitate immediately formed, and the reaction was stirred on ice for 10 minutes. The ice bath was removed and the reaction was stirred overnight, warming to room temperature. TLC (ninhydrin) confirmed reaction completion. The reaction was dry loaded with silica onto a 24 g silica gel column and purified with flash chromatography (EtOAc/hexanes, 0-70% over 35 min, ELS detection). After this duration, the column was flushed with 20% MeOH/DCM to expedite elution of product. Fractions were pooled and concentrated under reduced pressure to give 1 (602 mg) as a white solid in 84% yield. LC/MS (ESI⁺) calculated m/z 464.40 (M), found 465.64 (M+H⁺).

To a 40 mL vial with stir bar was added intermediate 1 (165 mg, 0.355 mmol) and 4 M HCl in dioxanes (30 mL, 128.8 mmol). The vial was tightly capped and stirred for 2 h. The reaction immediately turned orange and then a white precipitate formed within 10 minutes. Reaction completion was confirmed by LCMS. The reaction was concentrated under reduced pressure, azeotroped with PhMe (5 mL), and dried overnight to give 2 (142 mg) as a white solid HCl salt in quantitative yield. Intermediate 2 (142 mg, 0.354 mmol) was suspended in DCM (10 mL) and triethylamine (0.15 mL, 1.06) and stir bar were added. NHS-PEG₂-NHBoc (145 mg) was added last and the reaction was stirred for 2 h under N2. Reaction completion was confirmed by LCMS. The reaction was diluted with DCM and washed with saturated NaHCO₃ (×2), brine, dried over MgSO₄, filtered, and concentrated under reduced pressure to give crude material that was dissolved in DCM (8 mL) and dry loaded onto a 12 g silica column and purified with flash chromatography (MeOH/DCM 0-5%, ELS detection) to give intermediate 3 (172 mg) as a white solid in 78% yield. LC/MS (ESI⁺) calculated m/z 623.49 (M), found 624.78 (M+H⁺).

Intermediate 3 (170 mg, 0.272 mmol) was stirred in 4 M HCl in dioxane (7 mL, 27.2 mmol) in a sealed RB flask for 2 h. LCMS confirmed reaction completion. The reaction was concentrated under reduced pressure, azeotroped with PhMe (5 mL), and dried overnight to give 4 (154 mg) as a white solid HCl salt in quantitative yield. LC/MS (ESI⁺) calculated m/z 523.43 (M), found 524.60 (M+H⁺).

To an oven dried 40 mL with stir bar and under N2 was added intermediate 4 (75 mg, 0.135 mmol), sieve dried THF (3 mL), and triethylamine (0.12 mL, 0.815 mmol). 2,3,4,5,6-Pentafluorophenyl 4-[5-(methylsulfonyl)-1,3,4-oxadiazol-2-yl]benzoateSulfone (71 mg, 0.0163 mmol) was added and the reaction was stirred for 2 h. The reaction slowly became very heterogeneous. A small aliquot was taken, diluted with MeCN, and analyzed with LCMS to confirm reaction completion. The reaction was dry loaded with celite onto a 12 g silica gel column and purified with flash chromatography (MeOH/DCM 0-8% over 45 min, ELS detection) to give LP-361-p (52 mg) as a white solid in 52% yield. LC/MS (ESI⁺) calculated m/z 773.44 (M), found 775.11 (M+H⁺).

### Synthesis of LP-371-p

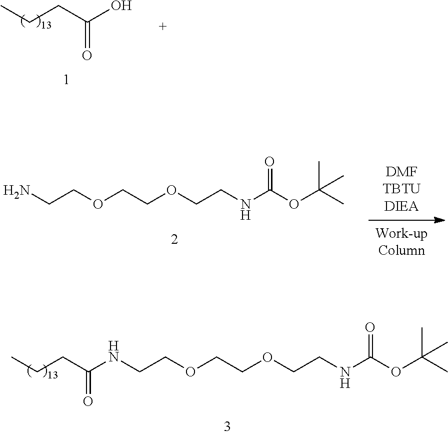

Compound 1 (palmitic acid, 2.50 g) was dissolved in 60 mL DMF. Then TBTU (3.44 g) and DIPEA (6.9 mL) were added. The reaction was stirred for 10 minutes then compound 2 (2.66 g in DMF) was added. The reaction was complete after 1 hour. The mixture was diluted with 300 mL EtOAc and washed with 3% citric acid (3×60 mL), H₂O (2×60 mL), and NaCl (1×60 mL), then dried over Na₂SO₄. The product was filtered and concentrated on rotary evaporator and high vacuum. The product was purified using column chromatography (loaded in DCM (15 mL) with a drop of MeOH onto a 80G RediSep Gold Rf column, mobile phase MeOH/DCM, 0-5% over 30 minutes. Yield 4.094 g. LC-MS: calculated [M+H] 486.74, found 488.11.

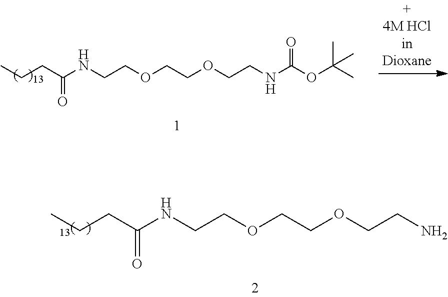

Compound 1 (4.094 g) was dissolved in 4M HCl in dioxane (28 mL) at 0° C. for 10 minutes. The reaction was allowed to warm to room temperature then stirred for 2 hours. The product was concentrated on rotary evaporator and high vacuum. Yield 3.485 g. LC-MS: calculated [M+H] 386.57, found 388.02.

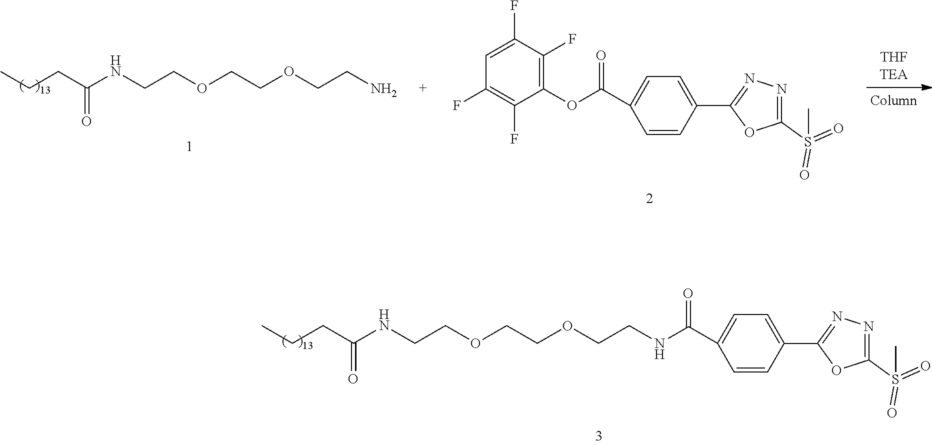

Compound 1 (2.3 g) was dissolved in 80 mL THF. Then TEA (4.975 mL) and compound 2 (3.10 g) were added. The reaction was stirred for 1 hour. The reaction was dry loaded with Celite 545, the mixture was concentrated in a 28° C. water bath and placed on high vacuum to fully dry. The product was purified with flash chromatography (MeOH/DCM, 0-6% over 40 min.) Yield 2.905 g. LC-MS: calculated [M+H] 636.85, found 637.95.

### Synthesis of LP-374-p

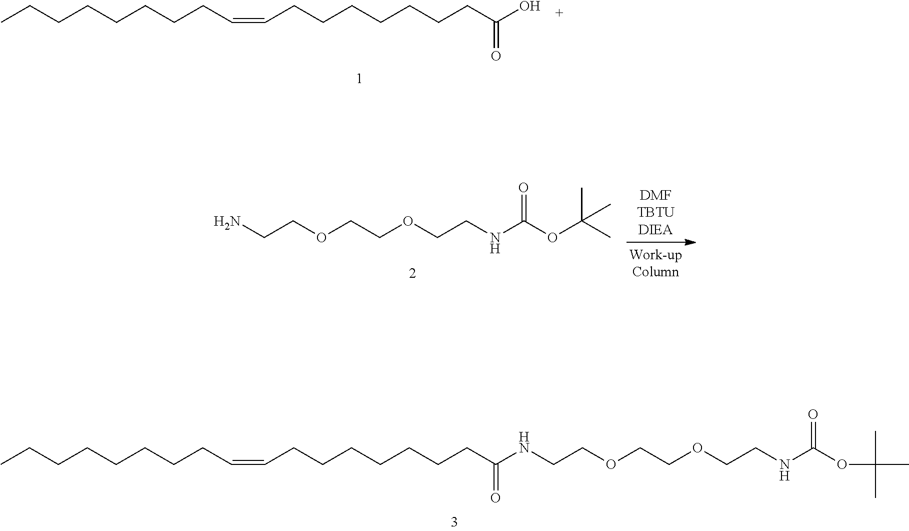 [SMILES: CCCCCCCC/C=C\CCCCCCCC(=O)O ; CC(C)(C)OC(=O)NCCOCCOCCN ; CCCCCCCC/C=C\CCCCCCCC(=O)NCCOCCOCCNC(=O)OC(C)(C)C]

Compound 1 (oleic acid, 200 mg) was dissolved in 6.5 mL DMF. Then TBTU (250 mg) and DIPEA (0.501 mL) were added to the reaction mixture. The reaction was stirred for 10 minutes then compound 2 (193 mg in DMF) was added. The reaction was complete after 1 hour. The reaction was diluted with 75 mL EtOAc and washed with 3% citric acid (3×8 mL), H₂O (2×8 mL), NaCl (1×8 mL), dried over Na₂SO₄, filtered and concentrated with rotary evaporator and placed under high vacuum. The product was purified on column (loaded in 1 mL DCM onto 12G column, MeOH/DCM, 0-3% over 30 min). Yield 254 mg. LC-MS: calculated [M+H] 512.76, found 513.62.

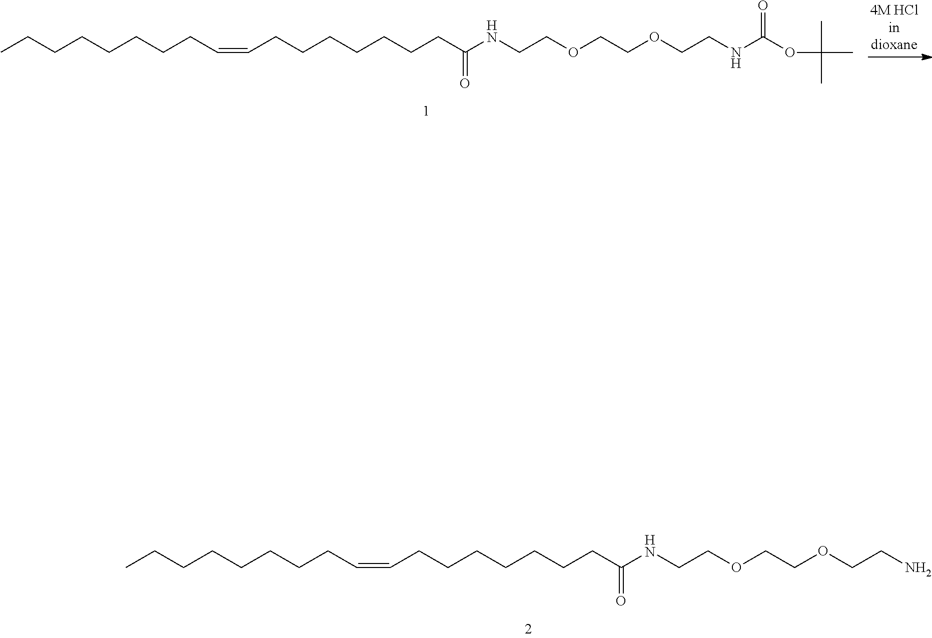

Compound 1 (150 mg) was dissolved in 4M HCl in dioxane at 0° C. The mixture was allowed to warm to room temperature and the reaction was stirred for 90 minutes. The product was concentrated on rotary evaporator and further dried under high vacuum. Yield 110 mg. LC-MS: calculated [M+H] 412.66, found 413.35.

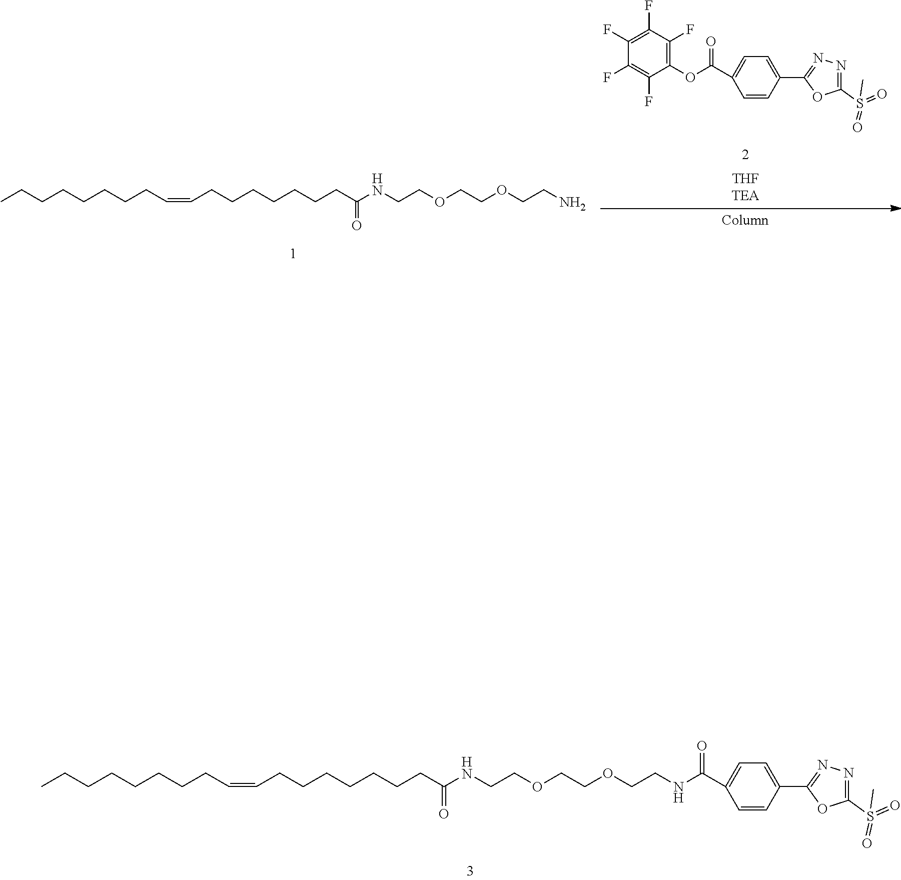 [SMILES: CS(=O)(=O)c1nnc(-c2ccc(C(=O)Oc3c(F)c(F)c(F)c(F)c3F)cc2)o1 ; CCCCCCCC/C=C\CCCCCCCC(=O)NCCOCCOCCN ; CCCCCCCC/C=C\CCCCCCCC(=O)NCCOCCOCCNC(=O)c1ccc(-c2nnc(S(C)(=O)=O)o2)cc1]

Compound 1 (110 mg) was dissolved in 4.5 mL THF. Then compound 2 (145 mg) and TEA (0.223 mL) were added. The reaction was stirred for 1 hour. The mixture was concentrated in a 28° C. water bath, dissolved in sieve dried DCM, loaded onto column, and purified with flash chromatography (dry MeOH/DCM, 0-8% over 30 min). Yield 92 mg. LC-MS: calculated [M+H] 662.86, found 664.12.

### Synthesis of LP-375-p

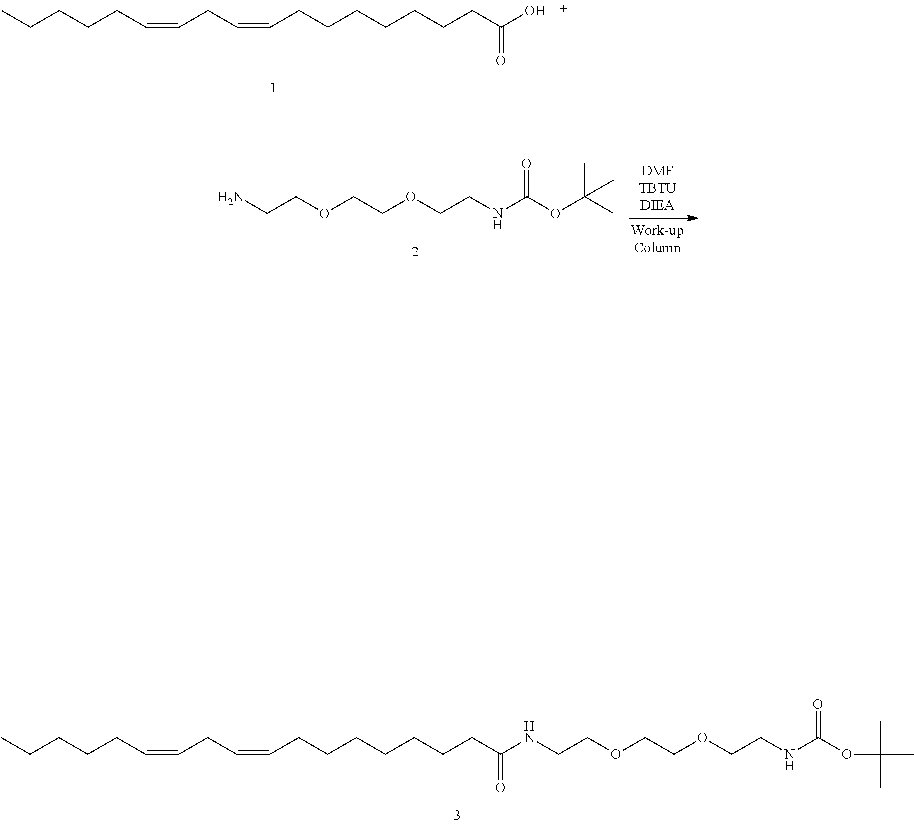 [SMILES: CCCCC/C=C\C/C=C\CCCCCCCC(=O)O ; CC(C)(C)OC(=O)NCCOCCOCCN ; CCCCC/C=C\C/C=C\CCCCCCCC(=O)NCCOCCOCCNC(=O)OC(C)(C)C]

Compound 1 (linoleic acid, 200 mg) was dissolved in 6.5 mL DMF. Then TBTU (252 mg) and DIPEA (0.505 mL) were added and the mixture was stirred for 10 minutes. Then compound 2 (195 mg) was added and the reaction was stirred for 1 hour. The mixture was then diluted with 75 mL EtOAc and washed with 3% citric acid (3×8 mL), H₂O (2×8 mL), NaCl (1×8 mL), dried over Na₂SO₄, filtered and concentrated on rotary evaporator and placed under high vacuum. The product was loaded onto 12G column in 1 mL DCM and purified with flash chromatography (MeOH/DCM, 0-4% over 30 min). Yield 266 mg. LC-MS: calculated [M+H] 510.76, found 511.90.

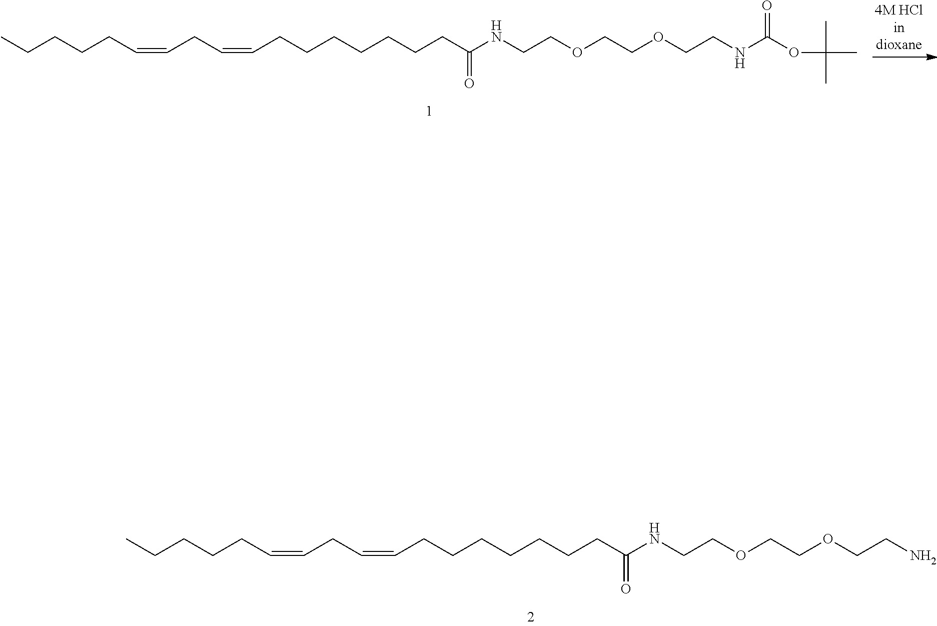

Compound 1 (166 mg) was dissolved in 2.5 mL of 4M HCl in dioxane. The mixture was stirred for 1 hour. The product was concentrated on rotary evaporator and placed under high vacuum. Yield 125 mg. LC-MS: calculated [M+H] 410.64, found 411.18.

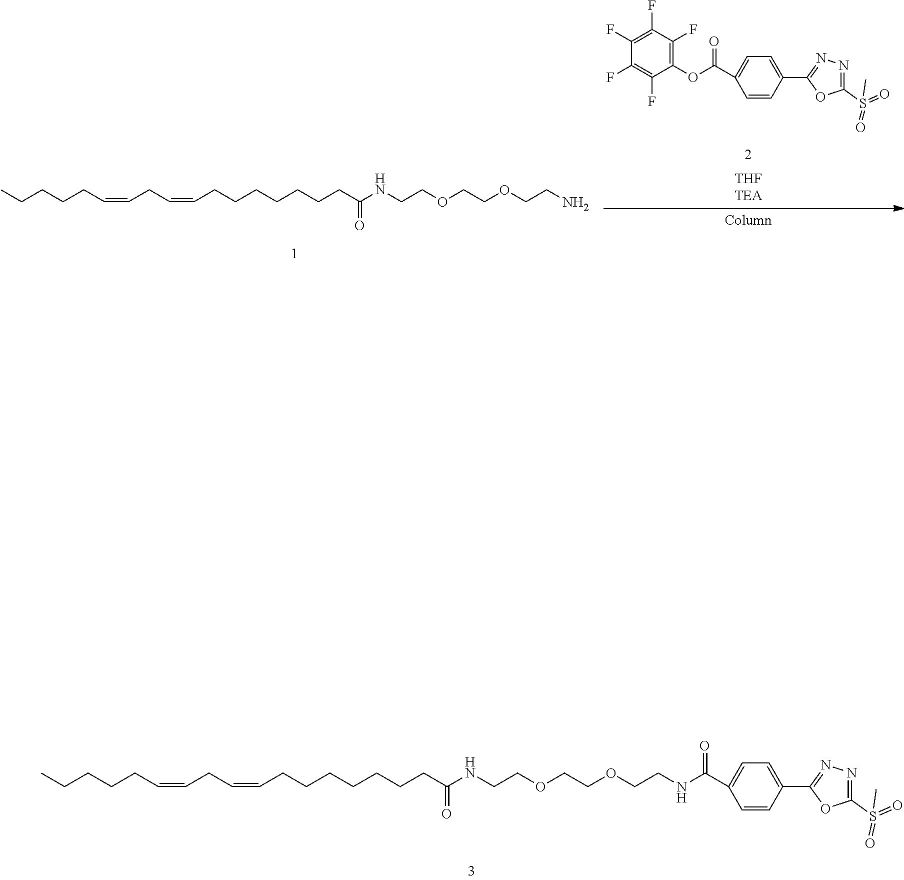 [SMILES: CS(=O)(=O)c1nnc(-c2ccc(C(=O)Oc3c(F)c(F)c(F)c(F)c3F)cc2)o1 ; CCCCC/C=C\C/C=C\CCCCCCCC(=O)NCCOCCOCCN ; CCCCC/C=C\C/C=C\CCCCCCCC(=O)NCCOCCOCCNC(=O)c1ccc(-c2nnc(S(C)(=O)=O)o2)cc1]

Compound 1 (125 mg) was dissolved in 4.5 mL of THF. Then compound 2 (165 mg) and TEA (0.255 mL) were added. The mixture was stirred for 1 hour. The compound was concentrated in a 28° C. water bath, loaded with sieve dried DCM onto column, and purified with flash chromatography (dry MeOH/DCM, 0-8% over 25 minutes). Yield 110 mg. LC-MS: calculated [M+H] 660.87, found 662.05.

### Synthesis of LP-377-p

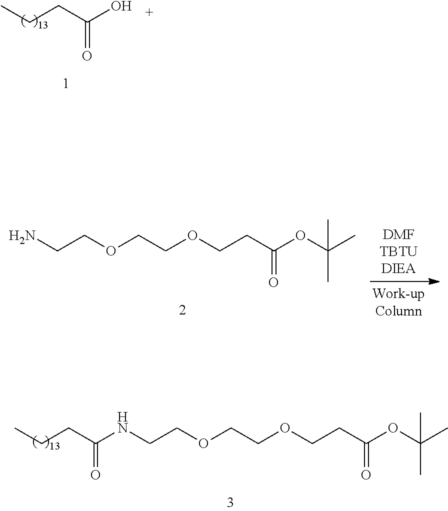

Compound 1 (palmitic acid, 175 mg) was dissolved in 6.5 mL DMF. Then TBTU (252 mg) and DIPEA (0.483 mL) were added. The mixture was stirred for 10 minutes. Then compound 2 (183 mg in DMF) was added. The reaction was stirred for 1 hour. Then the mixture was diluted with 75 mL of EtOAc and washed with 3% citric acid (3×8 mL), H₂O (2×8 mL), NaCl (1×8 mL); then dried over Na₂SO₄, filtered and concentrated on rotary evaporator and placed under high vacuum. The crude product was wet loaded in 2 mL DCM onto 12G column and purified with flash chromatography (MeOH/DCM, 0-3% over 30 minutes). Yield 277 mg. LC-MS: calculated [M+H] 471.72, found 472.57.

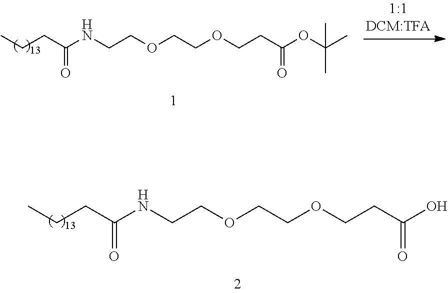

Compound 1 (277 mg) was dissolved in 6 mL 1:1 DCM:TFA. The reaction was stirred for 1 hour. The product was concentrated on rotary evaporator and placed under high vacuum. Yield 249 mg. LC-MS: calculated [M+H] 415.62, found 416.22.

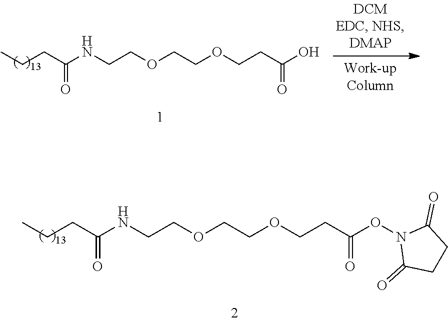

Compound 1 (244 mg) was dissolved in 10 mL DCM. Then EDC HCl (141 mg), NHS (135 mg), and DMAP (14 mg) were added consecutively. The reaction was allowed to stir overnight at room temperature. The reaction mixture was then diluted with 70 mL DCM and washed with citric acid (pH 3, 3×8 mL), then NaCl (1×8 mL with a drop of 10% citric acid solution), dried over Na₂SO₄, filtered and concentrated in vacuo. The crude product was loaded in 2.5 mL DCM onto a 12G column and purified with flash chromatography (MeOH/DCM, 0-3% over 30 minutes). Yield 63 mg. LC-MS: calculated [M+H] 512.69, found 513.53.

### Synthesis of LP-378-p

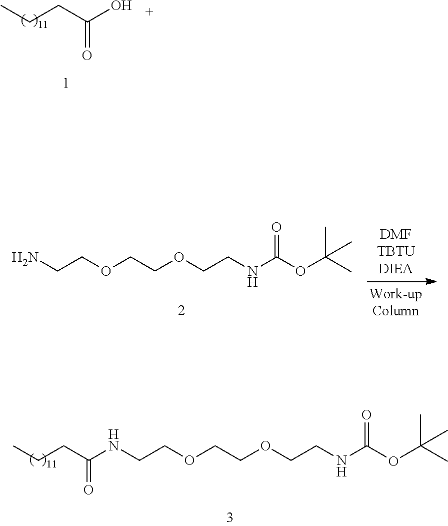

Compound 1 (myristic acid, 2.50 g) was dissolved in 50 mL DMF. Then TBTU (3.87 g) and DIPEA (7.7 mL) were added and the mixture was stirred for 10 minutes. Then compound 2 (2.99 g in DMF) was added and the reaction was stirred for 1 hour. The reaction mixture was diluted with 300 mL EtOAc and washed with 3% citric acid (3×60 mL), H₂O (2×60 mL), and NaCl (1×60 mL), dried over Na₂SO₄, filtered and concentrated on rotary evaporator and placed under high vacuum. The crude product was loaded onto a 80G RediSep Gold Rf column in 15 mL DCM and purified with flash chromatography (MeOH/DCM, 0-4% over 30 min. Yield 4.172 g. LC-MS: calculated [M+H] 458.68, found 459.83.

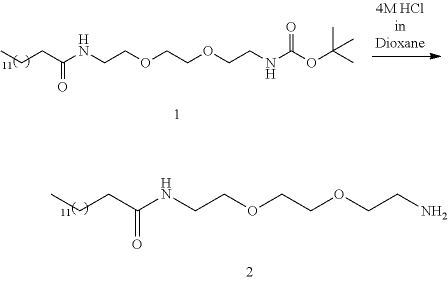

Compound 1 (4.172 g) was dissolved in 28 mL 4M HCl in dioxane at 0° C. and the mixture was allowed to stir for 10 minutes. Then the reaction was allowed to warm to room temperature and stirred for 2 hours. The product was concentrated on rotary evaporator and placed under high vacuum. Yield 3.436 g. LC-MS: calculated [M+H] 358.57, found 359.76.

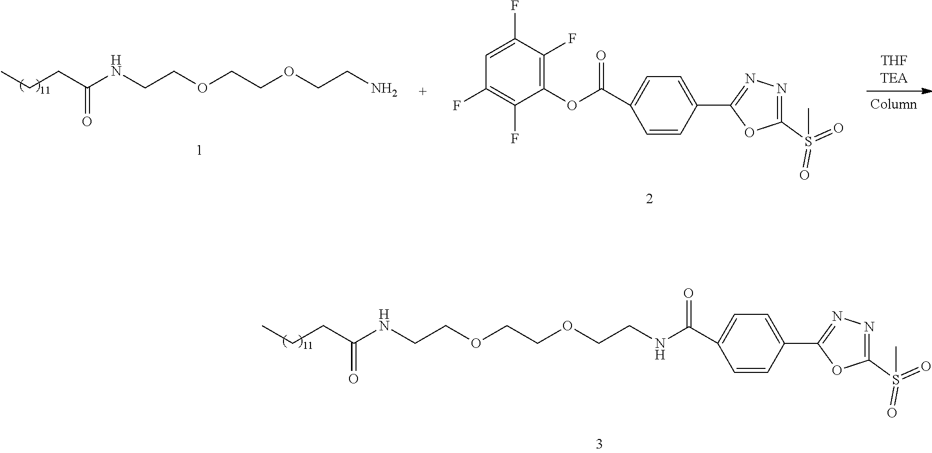

Compound 1 (2.5 g) was dissolved in 80 mL THF. Then TEA (5.83 mL) and compound 2 (3.48 g) were added. The reaction was allowed to stir for 1 hour. Celite® 545 was added and the mixture was concentrated in a 28° C. water bath and placed under high vacuum. The compound was dry loaded on 120G column and purified with flash chromatography (MeOH/DCM, 0-7% over 40 min.) Yield 2.33 g. LC-MS: calculated [M+H] 608.80, found 610.12.

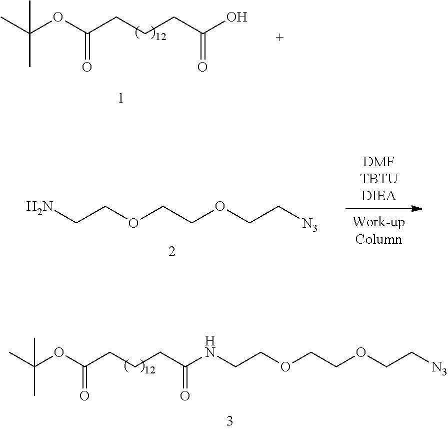

Compound 1 (Asta Tech® #89929, 3.00 g) was dissolved in 50 mL DMF. Then TBTU (3.09 g) and DIPEA (6.2 mL) were added and the mixture was stirred for 10 minutes. Compound 2 (1.68 g in DMF) was then added. The mixture was allowed to stir for 1 hour. Then the mixture was diluted with 300 mL EtOAc and washed with 3% citric acid (3×60 mL), H₂O (2×60 mL), and NaCl (1×60 mL), dried over Na₂SO₄, filtered and concentrated on rotary evaporator and placed under high vacuum. The crude product was loaded onto 80 g column in 14 mL DCM with 2 drops MeOH and purified with flash chromatography (MeOH/DCM, 0-4% over 40 min.) Yield 4.033 g. LC-MS: calculated [M+H] 498.71, found 499.85.

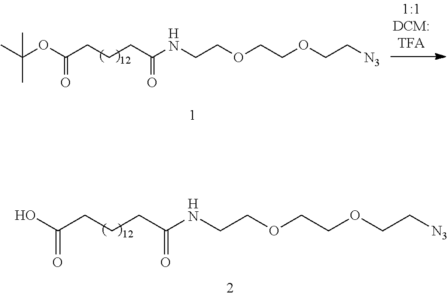

Compound 1 (4.033 g) was dissolved in 50 mL 1:1 DCM:TFA. The mixture was stirred for 1 hour. The product was concentrated on rotary evaporator and placed under high vacuum. The product was then dissolved in ACN, concentrated, then dissolved in DCM and concentrated again. Yield 3.839 g. LC-MS: calculated [M+H] 442.60, found 443.81.

### Synthesis of LP-380-p

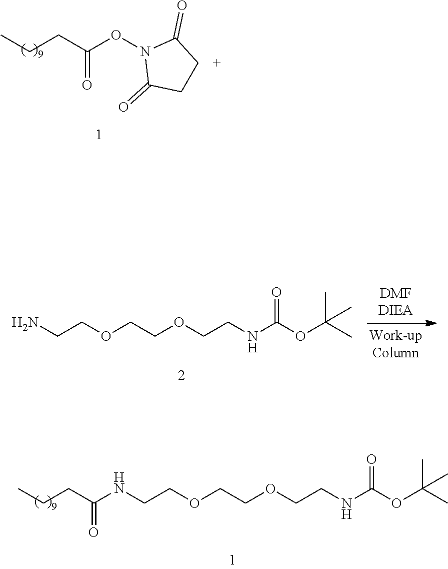

Compound 1 (lauric acid NHS ester, 150 mg) was dissolved in 6 mL DMF. Then DIPEA (0.357 mL) and compound 2 (138 mg) were added and the reaction was stirred for 1 hour. The mixture was diluted with 75 mL EtOAc and washed with 3% citric acid in water (3×8 mL), H₂O (2×8 mL), and NaCl (1×8 mL), then dried over Na₂SO₄, filtered and concentrated on rotary evaporator and placed under high vacuum. The crude product was loaded in 2 mL DCM onto a 12G column and purified with flash chromatography (MeOH/DCM, 0-4% over 30 min.) Yield 177 mg. LC-MS: calculated [M+H] 430.63, found 431.43.

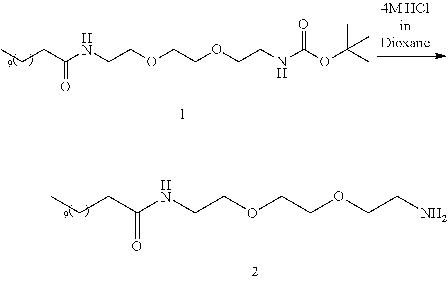

Compound 1 (177 mg) was dissolved in 4 mL 4 M HCl in dioxane at 0° C. and stirred for 10 minutes. Then the mixture was allowed to warm to room temperature and stirred for 1 hour. The product was concentrated on rotary evaporator and placed under high vacuum, then dissolved and concentrated twice with DCM/toluene and placed under high vacuum. Yield 150 mg. LC-MS: calculated [M+H] 330.51, found 331.16.

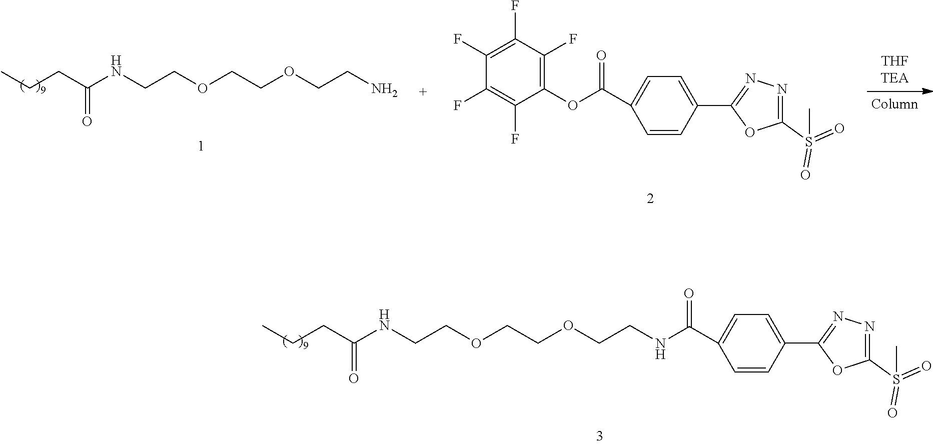

Compound 1 (150 mg) was dissolved in 6 mL THF, then TEA (0.380 mL) and compound 2 (246 mg) were added. The reaction was stirred for 1 hour. Then Celite® 545 was added and the mixture was concentrated in a 28° C. water bath. The crude product was dry loaded onto a 12G column and purified with flash chromatography (MeOH/DCM, 0-4% over 25 min.) Yield 148 mg. LC-MS: calculated [M+H] 580.74, found 581.94.

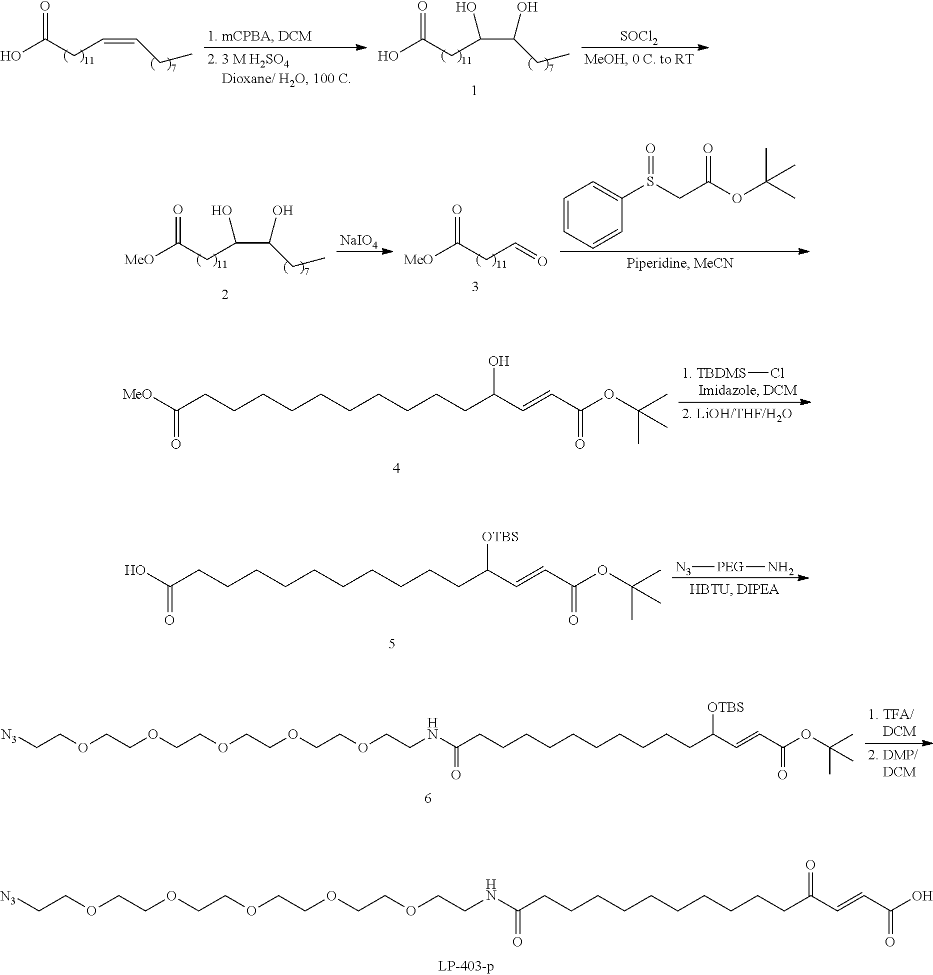

To a 250 mL RB flask with stir bar and under N2 was added erucic acid (5 g, 14.7 mmol) and dry DCM (125 mL). The reaction was cooled in an ice bath for 10 min and then mCPBA (77%, 4.3 g, 19.1 mmol) was added in portions over 5 min. The reaction was stirred on ice for 10 min and then the ice bath was removed, and the reaction was stirred for 18 h warming to room temperature. NMR confirmed reaction completion. The reaction was directly dry loaded with silica onto a 220 g silica column and purified with flash chromatography (MeOH/DCM, 0-5%) to give intermediate epoxide (4.4 g) as a white solid in 83% yield. To a 200 mL RB flask with stir bar was added the intermediate epoxide (2.5 g, 7.04), dioxane (36 mL), and H₂O (12 mL). H2SO₄ (3 M solution in H₂O, 24 mL, 70.2 mmol) was added and the reaction was stirred at 100° C. (oil bath temperature) for 18 h. HPLC with ELSD verified reaction completion. The reaction was diluted with H₂O (50 mL) and extracted with EtOAc (150 mL×3). The pooled organic layers were washed with brine, dried over MgSO₄, filtered, and concentrated under reduced pressure to give 1 (2.5 g) as a white solid in 95% yield. LC/MS (ESI−) calculated m/z 372.32 (M), found 371.48−(M−H⁺).

To an oven dried 500 mL RB flask with stir bar and under N₂ was added intermediate 1 (5.4 g, 14.5 mmol) and sieve dried MeOH (200 mL). The resulting suspension was cooled in an ice bath for 10 min and then thionyl chloride (1.1 mL, 15.2 mmol) was added dropwise over 1 min. The reaction was stirred on ice for 10 min and then the ice bath was removed, and the reaction was stirred for 2 h warming to room temperature. HPLC with ELSD confirmed reaction completion. The reaction was concentrated down to a small volume (25 mL), diluted with DCM, and washed with saturated NaHCO₃ (×2), brine, dried over MgSO₄, filtered, and concentrated under reduced pressure to give 2 (5.4 g) as an oil in 97% yield. ¹H NMR (400 MHz, CDCl₃) δ=3.65 (s, 3H), 3.37-3.41 (m, 2H), 2.29 (t, 2H), 1.99 (br. s., 2H), 1.66-1.54 (m, 3H), 1.54-1.37 (m, 5H), 1.37-1.20 (overlapping m, 26H), 0.87 (t, 3H).

To a 500 mL RB flask with stir bar was added intermediate 2 (5.4 g, 13.9 mmol), THF (150 mL), and H₂O (50 mL). NaIO₄ (4.4 g, 20.9 mmol) was added and the reaction was stirred for 18 h. HPLC with ELSD confirmed reaction completion. The reaction was concentrated under reduced pressure to remove THF, further diluted with H₂O (50 mL), and extracted with DCM (100 mL×2). The pooled organic layers were washed with brine, dried over MgSO₄, filtered, and concentrated under reduced pressure to give crude material that was dissolved in DCM (20 mL) and loaded onto a 120 g silica gel column and purified with flash chromatography (EtOAc/hexanes, 0-10% over 60 min, ELS detection) to give 3 (1.6 g) as a colorless oil in 50% yield. ¹H NMR (400 MHz, CD₂Cl₂) δ=9.73 (t, 1H), 3.63 (s, 3H), 2.40 (dt, 2H), 2.29 (t, 2H), 1.65-1.57 (m, 2H), 1.36-1.26 (m, 16H).

To the RB flask already containing 3 (1.6 g) and under N2 was added stir bar, piperidine (0.85 mL, 8.57 mmol), and sieve dried MeCN (60 mL). 1,1-Dimethylethyl 2-(phenylsulfinyl)acetate (1.74 g, 7.25 mmol) was added and the reaction was stirred for 18 h. HPLC with ELSD confirmed reaction completion. The reaction was concentrated under reduced pressure, dissolved in DCM (20 mL), loaded onto a 120 g silica gel column, and purified with flash chromatography (EtOAc/hexanes, 0-30% over 60 min) to give 4 (1.8 g) as a colorless oil in 77% yield. ¹H NMR (400 MHz, CDCl₃) δ 6.83 (dd, 1H), 5.94 (dd, 1H), 4.29-4.24 (m, 1H), 3.66 (s, 3H), 2.29 (t, 2H), 1.65-1.52 (m, 4H), 1.48 (s, 9H), 1.32-1.23 (br. s., 14H).

To a 100 mL RB flask with stir bar and under N2 was added intermediate 4 (800 mg, 2.24 mmol), DCM (210 mL), and imidazole (381 mg, 5.6 mmol). The resulting solution was cooled in an ice bath for 10 min and then TBDMS-Cl (405 mg, 2.69 mmol) was added. The reaction was stirred on ice for 10 min and then the ice bath was removed, and the reaction was stirred for 16 h warming to room temperature. HPLC with ELSD confirmed reaction completion. The reaction was diluted with DCM, washed with H₂O (×2), brine, dried over MgSO₄, filtered, and concentrated under reduced pressure to give crude material that was dissolved in DCM (4 mL) and loaded onto a 40 g silica gel column and purified with flash chromatography (EtOAc/hexanes, 0-10% over 30 min, ELS detection) to give silyl protected intermediate (447 mg) as a colorless oil in 43% yield. A small portion of this intermediate (105 mg, 0.22 mmol) was added to a 40 mL vial and THF (1.5 mL), H₂O (0.5 mL), and LiOH (1 M solution in H₂O, 0.512 mL, 0.512 mmol) was added consecutively. The reaction was stirred for 12 h. HPLC with ELSD confirmed reaction completion. The THF was removed under reduced pressure, the reaction was acidified with 10% citric acid (1 mL) and extracted with EtOAc (x 2). The pooled extracts were washed with brine, dried over MgSO₄, filtered, and concentrated under reduced pressure to give 5 (82 mg) as a colorless oil in 82% yield. To a 40 mL vial with stir bar and under N2 was added 5 (335 mg, 0.733 mmol), azido-PEG₅-amine (270 mg, 0.88 mmol), DIPEA (0.38 mL, 2.2 mmol), and sieve dried DCM (12 mL). HBTU (334 mg, 0.88 mmol) was added and the reaction was stirred at room temperature for 3 h. Both LCMS and HPLC with ELSD were utilized to confirm reaction completion. The reaction was diluted with DCM, washed with saturated NaHCO₃ (×4), brine, dried over MgSO₄, filtered, and concentrated under reduced pressure to give crude material that was dissolved in DCM (5 mL) and loaded onto a 24 g silica gel column and purified with flash chromatography MeOH/DCM (0-5% over 30 min, ELS detection, product eluted at 2%) to give 6 (401 mg) as a colorless oil in 73% yield. LC/MS (ESI⁺) calculated m/z 744.51 (M), found 745.86 (M+H⁺).

To a 40 mL vial with stir bar and under N2 was added 6 (95 mg, 0.134 mmol), DCM (1 mL), and TFA (1.25 mL, 11.1 mmol). The reaction was stirred at room temperature for 4.5 h. Both LCMS and HPLC with ELSD confirmed reaction completion. The reaction was concentrated under reduced pressure, dissolved in DCM (5 mL), concentrated again, and then dried on vacuum pump for 1 h to give acid intermediate (73 mg) that was used without purification. The 40 mL vial containing acid intermediate (73 mg, 0.127 mmol) was placed under N2 and dissolved in sieve dried DCM (1.5. mL). The reaction was cooled in an ice bath for 10 min and then Dess Martin periodinane (57 mg, 0.133 mmol) was added. The reaction was stirred on ice for 30 min and then the ice bath was removed, and the reaction was stirred for 3 h warming to room temperature. LCMS confirmed reaction completion. The reaction was suspended in 10% MeOH/DCM, dry loaded with silica onto a 12 g silica gel column, and purified with flash chromatography (MeOH/DCM 0-10%, product eluted around 5%) to give LP-403-p (11.3 mg) as a white solid in 16% yield. LC/MS (ESI⁺) calculated m/z 572.34 (M), found 573.56 (M+H⁺).

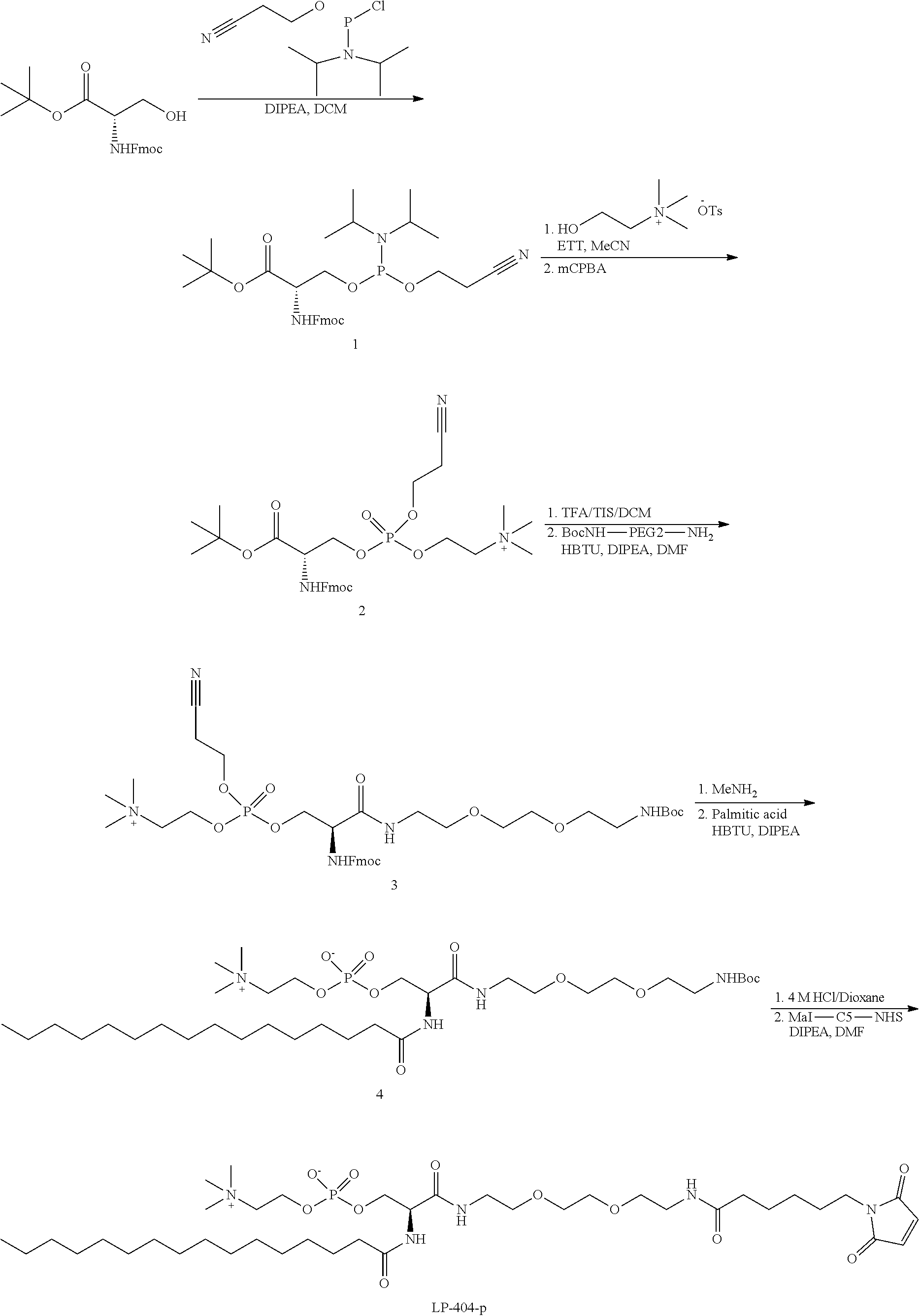

To an oven dried 100 mL round bottom flask with stir bar and under N2 was added Fmoc-Ser-OtBu (3 g, 7.8 mmol), sieve dried DCM (25 mL), DIPEA (3.4 mL, 19.5 mmol), and activated molecular sieves. The resulting solution was cooled in an ice bath for 10 min and then 2-cyanoethyl N,N-diisopropylchlorophosphoramidite (2.3 mL, 10.1 mmol) was added dropwise over 1 min. The reaction was gently stirred on ice for 15 min and then the ice bath was removed, and the reaction was gently stirred for 2 h warming to room temperature. LCMS confirmed reaction completion. The reaction was quenched with MeOH (10 mL), filtered (to remove sieves), and concentrated under reduced pressure to give a crude yellow oil that was dissolved in DCM (20 mL), loaded onto a 120 g silica gel column, and purified with flash chromatography (EtOAc/hexanes with 1% Et₃N, gradient 10-80% over 40 min). Pooled product fractions were concentrated and azeotroped with PhMe (10 mL) to give 1 (3.29 g) as a colorless oil in 73% yield. LC/MS (ESI−) calculated m/z 583.28 (M), found 584.91 (M+H⁺).

The round bottomed flask containing intermediate 1 (3.29 g, 5.63 mmol) was placed under N₂, dissolved in sieve dried MeCN (10 mL) and 2-hydroxy-N,N,N-trimethylethanaminium 4-methylbenzenesulfonate (1.7 g, 6.2 mmol) was added as a solution in sieve dried MeCN (10 mL). ETT (0.75 M in MeCN, 7.24 mL, 5.63 mmol) was slowly added and the reaction was stirred under N₂ for 2 h. LCMS confirmed reaction completion. The reaction was cooled in an ice bath for 10 min and then mCPBA (77%, 2.47 g, 11.26 mmol) was added in portions. The reaction was stirred on ice for 15 min and then the ice bath was removed and the reaction was stirred for 1 h warming to room temperature. LCMS confirmed reaction completion. The reaction was concentrated under reduced pressure at 28° C. and dried on vacuum pump for 2 h to give a white oily solid. The solid was dissolved in DCM (20 mL, sonication required) and then loaded onto a 80 g silica gel column and purified with flash chromatography (MeOH/DCM, 0-20% over 40 min) to give 2 (1.66 g) as a colorless oil/tetrazolium salt in 40% yield. LC/MS (ESI⁺) calculated m/z 602.26 (M⁺), found 602.82 (M⁺).

The round bottom flask containing intermediate 2 (1.66 g, 2.14 mmol) was placed under N₂ and dissolved in sieve dried DCM (20 mL). Triisopropylsilane (1.3 mL, 6.41 mmol) and TFA (20 mL, 261 mmol) were added consecutively and the reaction was stirred for 2.5 h. LCMS confirmed reaction completion. The reaction was concentrated under reduced pressure, dissolved in DCM (15 mL) and concentrated again, and then azeotroped with PhMe (15 mL) to give intermediate acid (2.48 g) that was used without further purification. The round bottom flask containing crude intermediate acid (2.2 g) was placed under N₂ and dissolved in sieve dried DMF (25 mL). The reaction was cooled in an ice bath for 10 min and then DIPEA (0.625 mL, 3.60 mmol) and HBTU (1.36 g, 3.60 mmol) were added and the reaction was stirred on ice for 10 min. BocNH-PEG2-amine (893 mg, 3.60 mmol) was slowly added as a solution in DMF, the reaction was stirred for 10 min on ice, and then the ice bath was removed and the reaction was stirred for 7 h warming to room temperature. LCMS confirmed reaction completion. The reaction was concentrated under reduced pressure and then DMF (15 mL) was added and the reaction was concentrated under reduced pressure again. The crude reaction was dissolved in DCM (30 mL) and half of the reaction was loaded onto a 24 g silica gel column and purified with flash chromatography (MeOH/DCM, 0-20% over 1 h). The second half of the reaction was also purified the same way to give intermediate 3 (790 mg) as a colorless oil and TFA salt in 49% yield. LC/MS (ESI⁺) calculated m/z 776.36 (M⁺), found 777.09 (M⁺).

To a 40 mL vial with stir bar was added intermediate 3 (125 mg, 0.163 mmol) and then methylamine (40% in H₂O, 3.75 mL, 33.8 mmol) was added and the reaction was stirred for 2 h. LCMS confirmed reaction completion. The reaction was concentrated under reduced pressure and then azeotroped with PhMe (2 mL×2) to give crude product that was dissolved in sieve dried DCM/DMF (1:1, 3 mL). DIPEA (0.14 mL, 0.815 mmol), palmitic acid (73 mg, 0.285 mmol), and HBTU (109 mg, 0.285 mmol) were added consecutively and the reaction was stirred under N₂ for 16 h. LCMS confirmed reaction completion. The reaction was concentrated under reduced pressure, concentrated again with PhMe (10 mL), and dried on vacuum pump for 3 h. The crude material was dry loaded with silica onto a glass silica gel column and purified with manual flash column chromatography (MeOH/CHCl₃ 0-10% for 20 min, then switched to 15:9:1 CHCl₃/MeOH/H₂O for 30 min) to give 4 (42 mg) as an off yellow oil in 35% yield. LC/MS (ESI⁺) calculated m/z 738.49 (M), found 740.19 (M++H).

To a 40 mL vial was added stir bar, intermediate 4 (42 mg, 0.057 mmol), and 4 M HCl in dioxane (2 mL, 8 mmol). The reaction vial was capped tightly and stirred for 2 h. LCMS confirmed reaction completion. The reaction was concentrated under reduced pressure and then dried on vacuum pump for 3 h. The crude intermediate was dissolved in dry DMF (1 mL) and then DIPEA (30 μL, 0.171 mmol) and maleimide-C5-NHS ester (23 mg, 0.074 mmol) were added consecutively, and the reaction was stirred for 4.5 h. LCMS confirmed reaction completion. The reaction was concentrated under reduced pressure, concentrated again with PhMe (3 mL), and dried on vacuum pump for 3 h. The crude material was dry loaded with silica onto a glass silica gel column and purified with manual flash column chromatography (MeOH/CHCl₃ 0-30% for 25 min, then switched to 55:41:4 MeOH/CHCl₃/H₂O for 30 min) to give intermediate LP-404-p (22 mg) as a colorless oil in 50% yield. LC/MS (ESI⁺) calculated m/z 831.51 (M), found 833.27 (M+H⁺).

### Synthesis of LP-412-p

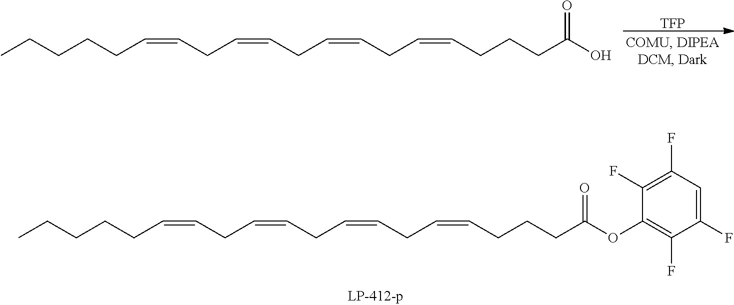 [SMILES: CCCCC/C=C\C/C=C\C/C=C\C/C=C\CCCC(=O)O ; "TFP" ; CCCCC/C=C\C/C=C\C/C=C\C/C=C\CCCC(=O)Oc1c(F)c(F)cc(F)c1F]

To a 40 mL vial with stir bar and under N₂ was added arachidonic acid (100 mg, 0.3218 mmol) and sieve dried DCM (3 mL). DIPEA (0.17 mL, 0.99 mmol), 2,3,5,6-tetrafluorophenol (66 mg, 0.394 mmol), and COMU (169 mg, 0.394 mmol) were added consecutively and the reaction was stirred for 3 h wrapped in foil. Reaction completion was confirmed by TLC (b-cresol green staining). The reaction was diluted with DCM and washed with H₂O (×3, or until aqueous extracts were colorless), brine, dried over MgSO₄, filtered, and concentrated under reduced pressure (shielding product from light) to give LP-412-p (156 mg) as a brown oil that was used without further purification. LC/MS (ESI⁺) calculated m/z 452.23 (M), found 454.04 (M+H⁺).

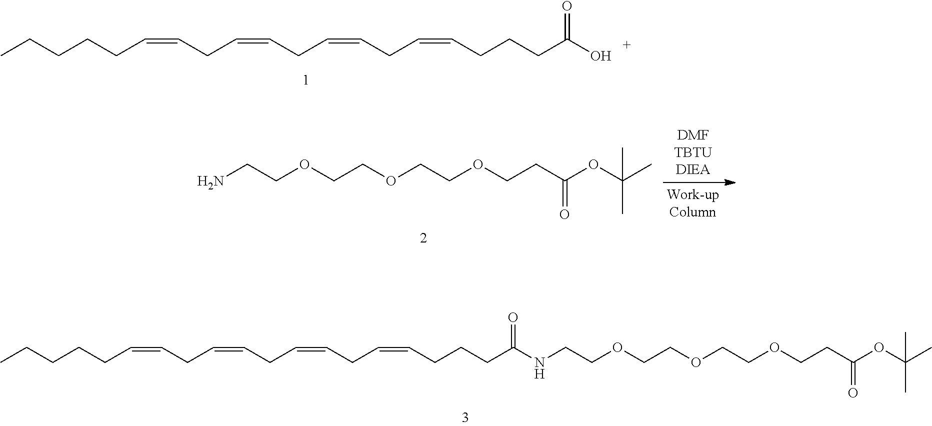 [SMILES: CCCCC/C=C\C/C=C\C/C=C\C/C=C\CCCC(=O)O ; CC(C)(C)OC(=O)CCOCCOCCOCCN ; CCCCC/C=C\C/C=C\C/C=C\C/C=C\CCCC(=O)NCCOCCOCCOCCC(=O)OC(C)(C)C]

Compound 1 (Asta Tech® #W15452, 1.00 g) was dissolved in 22 mL DMF. Then TBTU (1.212 g) and DIPEA (2.33 mL) were added. The mixture was stirred for 10 minutes, then compound 2 (1.05 g in DMF) was added. The flask was covered in foil and stirred for 1 hour. Then the mixture was diluted with EtOAc (225 mL), washed with 3% citric acid in water (3×30 mL), H₂O (2×30 mL), and NaCl (1×30 mL), the dried over Na₂SO₄, filtered and concentrated with rotary evaporator and placed under high vacuum. The crude product was loaded with DCM (4 mL) onto a 40G column and purified with flash chromatography (MeOH/DCM, 0-4% over 30 min). Yield 1.158 g. LC-MS: calculated [M+H] 563.82, found 564.83.

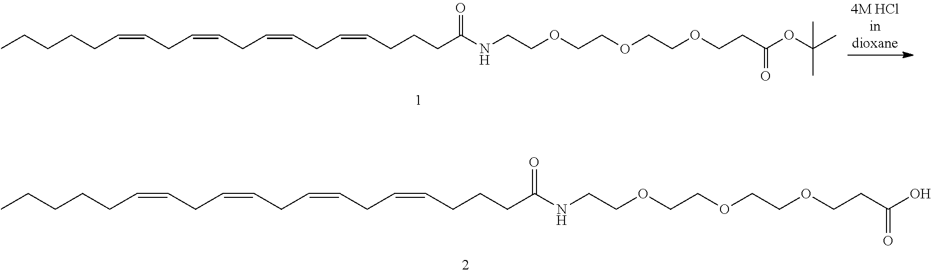

Compound 1 (1.158 g) was dissolved in 10.5 mL 4M HCl in dioxane at 0° C. The mixture was stirred in the dark for 10 minutes, then allowed to warm to room temperature and stirred for 4 hours wrapped in foil. The product was concentrated on rotary evaporator, then dissolved and concentrated twice using DCM/toluene and then placed under high vacuum. Yield 1.0135 g. LC-MS: calculated [M+H] 507.71, found 508.93.

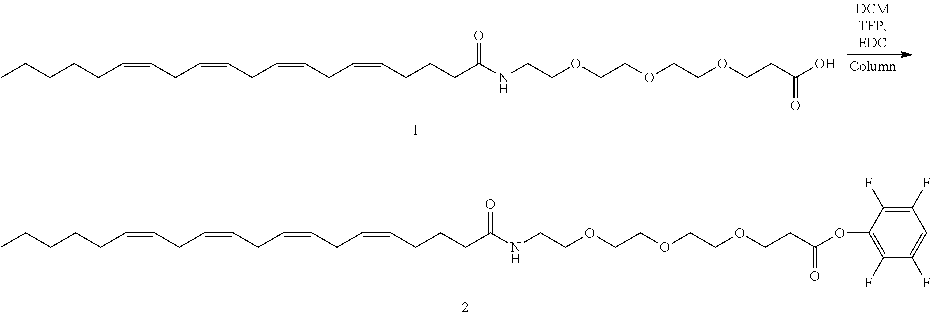 [SMILES: CCCCC/C=C\C/C=C\C/C=C\C/C=C\CCCC(=O)NCCOCCOCCOCCC(=O)O ; CCCCC/C=C\C/C=C\C/C=C\C/C=C\CCCC(=O)NCCOCCOCCOCCC(=O)Oc1c(F)c(F)cc(F)c1F]

Compound 1 (1.0135 g) was dissolved in 16 mL DCM. Then tetrafluorophenol (Sigma® #196789, 0.497 g) was added. After stirring for 10 minutes EDC-HCl (Sigma® #E7750, 0.574 g) was added. The mixture was allowed to stir for 2 hours. The reaction was concentrated in vacuo and the crude product was dry loaded with silica onto a 40G column and purified with flash chromatography (EtOAc/hexanes, 0-70% over 30 min.) Yield 655 mg. LC-MS: calculated [M+H] 655.77, found 656.90.

### Synthesis of LP-424-p

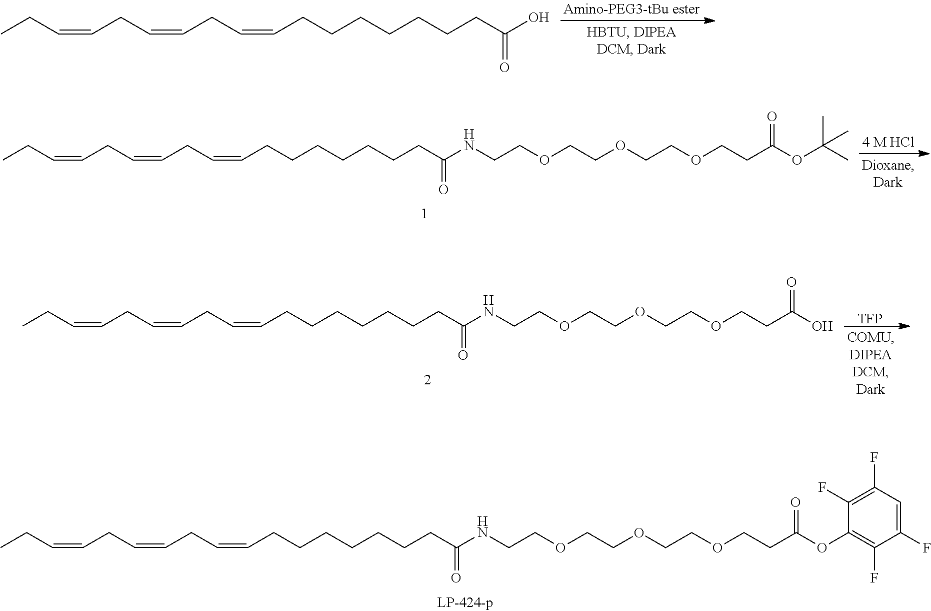

To a 40 mL vial with stir bar and under N₂ was added α-linolenic acid (200 mg, 0.718 mmol) and sieve dried DCM (4 mL). Amine-PEG-tert-butyl ester (239 mg, 0.862 mmol), DIPEA (0.37 mL, 2.15 mmol), and HBTU (327 mg, 0.862 mmol) were added consecutively and the reaction was stirred for 5 h wrapped in foil. Reaction completion was confirmed by TLC (b-cresol green and KMnO₄ staining). The reaction was diluted with DCM and washed with H₂O (×3), saturated NaHCO₃ (×3), dried over MgSO₄, filtered, and concentrated under reduced pressure (shielding product from light) to give crude material that was dissolved in DCM (3 mL), loaded onto a 12 g silica gel column, and purified with flash chromatography (EtOAc/hexanes, 0-75% over 50 min) to give intermediate 1 (293 mg) as a colorless oil in 76% yield. LC/MS (ESI⁺) calculated m/z 537.40 (M), found 538.61 (M+H⁺).

To a 40 mL vial with stir bar was added intermediate 1 (155 mg, 0.288 mmol) and 4 M HCl in dioxane (3.5 mL, 14.4 mmol). The vial was tightly capped, wrapped in foil, and the reaction was stirred for 4 h. Reaction completion was confirmed by TLc (KMnO₄ stain) and LCMS. The reaction was concentrated under reduced pressure (shielding product from light) and dried on vacuum pump overnight (shielded from light) to give intermediate 2 (145 mg) as an oil that was placed under N₂ and dissolved in sieve dried DCM (3 mL). Triethylamine (0.12 mL, 0.864 mmol), 2,3,5,6-tetrafluorophenol (58 mg, 0.346 mmol), and COMU (148 mg, 0.346 mmol) were added consecutively and the reaction was stirred for 3 h. Reaction completion was confirmed with LCMS. The reaction was diluted with DCM, washed with H₂O (×3), saturated NaHCO₃ (×3), brine, dried over MgSO₄, filtered, and concentrated under reduced pressure (shielding product from light) to give crude material that was dissolved in DCM (4 mL) and loaded onto a 12 g column and purified with flash chromatography (EtOAc/hexanes 0-100%; product eluted at 60% EtOAc) to give LP-424-p (54 mg) as an orange oil in 30% yield. LC/MS (ESI⁺) calculated m/z 629.33 (M), found 630.53 (M+H⁺).

### Synthesis of LP-425-p

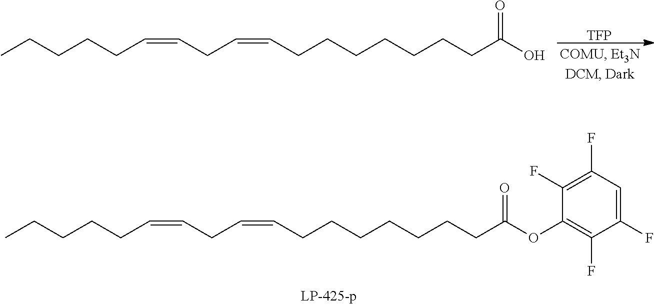 [SMILES: CCCCC/C=C\C/C=C\CCCCCCCC(=O)O ; "TFP" ; CCCCC/C=C\C/C=C\CCCCCCCC(=O)Oc1c(F)c(F)cc(F)c1F]

To a 40 mL vial with stir bar and under N₂ was added linoleic acid (100 mg, 0.356 mmol) and sieve dried DCM (3 mL). Triethylamine (0.15 mL, 1.06 mmol), 2,3,5,6-tetrafluorophenol (65 mg, 0.392 mmol), and COMU (167 mg, 0.392 mmol) were added consecutively and the reaction was stirred for 3 h wrapped in foil. Reaction completion was confirmed by TLC (b-cresol green staining). The reaction was diluted with DCM and washed with H₂O (×3 or until aqueous extracts were colorless), brine, dried over MgSO₄, filtered, and concentrated under reduced pressure (shielding product from light) to give LP-425-p (163 mg) that was used without further purification. LC/MS (ESI⁺) calculated m/z 428.51, found 429.55 (M+H⁺).

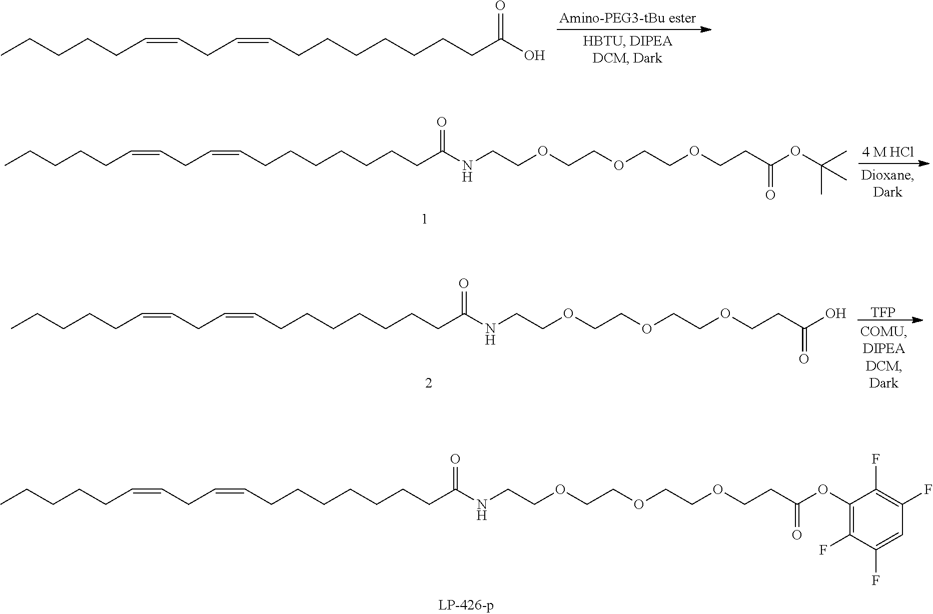

To a 40 mL vial with stir bar and under N₂ was added linoleic acid (100 mg, 0.356 mmol) and sieve dried DCM (3 mL). DIPEA (0.14 mL, 1.06 mmol), amine-PEG₃-tert-butyl ester (118 mg, 0.427 mmol), and HBTU (162 mg, 0.427 mmol) were added consecutively and the reaction was stirred for 4 h wrapped in foil. Reaction completion was confirmed by TLc (KMnO₄ staining). The reaction was diluted with DCM and washed with H₂O (×3), saturated NaHCO₃ (×3), brine, dried over MgSO₄, filtered, and concentrated under reduced pressure (shielding product from light) to give crude product that was dissolved in DCM (2.5 mL), loaded onto a 12 g silica gel column, and purified with flash chromatography (EtOAc/hexanes 0-75%; product eluted at 50% EtOAc) to give 1 (131 mg) as a colorless oil in 68% yield. The 40 mL vial already containing 1 (131 mg, 0.242 mmol) was stirred in 4 M HCl in dioxane (3.5 mL, 12.1 mmol) for 3.5 h wrapped in foil. Reaction completion was confirmed with TLC and LCMS. The reaction was concentrated under reduced pressure (shielding product from light) and dried on vacuum pump (shielding product from light) for 2 h to give 2 (117 mg, 0.242 mmol) as a colorless oil that was placed under N₂ and dissolved in sieve dried DCM (3 mL). Triethylamine (0.1 mL, 0.726 mmol), 2,3,5,6-tetrafluorophenol (44 mg, 0.266 mmol), and COMU (114 mg, 0.266 mmol) were added consecutively and the reaction was stirred for 1.5 h wrapped in foil. Reaction completion was confirmed by LCMS. The reaction was diluted with DCM, washed with H₂O (×3), saturated NaHCO₃ (×3), brine, dried over MgSO₄, filtered, and concentrated under reduced pressure (shielding product from light) to give crude material that was dissolved in DCM (3 mL), loaded onto a 12 g silica gel column, and purified with flash chromatography (EtOAc/hexanes, 0-100%) to give LP-426-p (49 mg) as a colorless oil in 32% yield. LC/MS (ESI⁺) calculated m/z 631.35 (M), found 633.21 (M+H⁺)

### Synthesis of LP-427-p

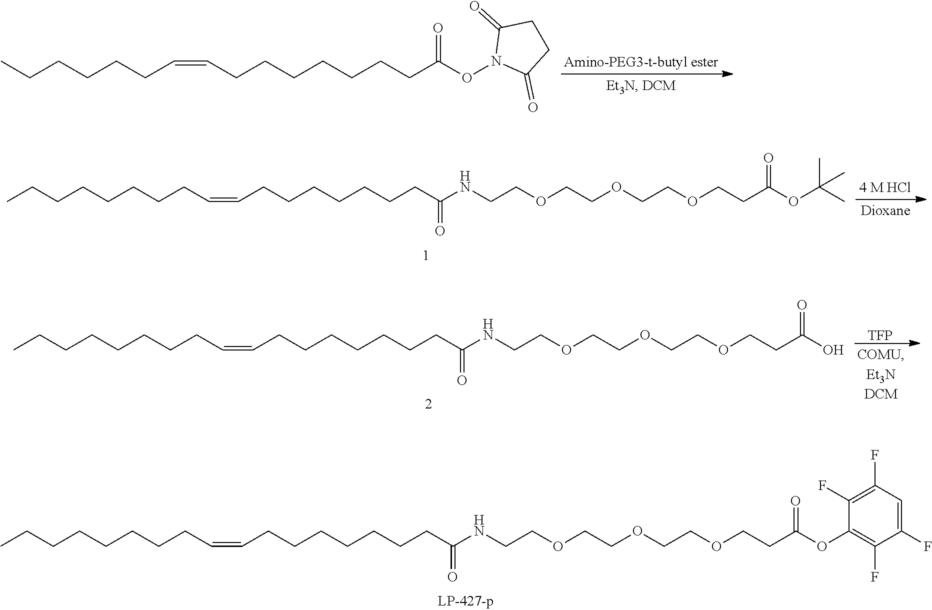

To a 40 mL vial with stir bar and under N₂ was added oleic acid NHS ester (100 mg, 0.284 mmol) and sieve dried DCM (2.5 mL). Triethyamine (0.12 mL, 0.852 mmol) and amino-PEG₃-tert-butyl ester (87 mg, 0.312 mmol) were added consecutively and the reaction was stirred for 3 h. Reaction completion was confirmed with TLc (KMnO₄ staining). The reaction was diluted with DCM and washed with saturated NaHCO₃ (×2), brine, dried over MgSO₄, filtered, and concentrated under reduced pressure to give crude product that was dissolved in DCM (2.5 mL), loaded onto a 12 g silica gel column, and purified with flash chromatography (EtOAc/hexanes 0-75% over 40 min; product eluted at 50% EtOAc) to give 1 (101 mg) as a colorless oil in 70% yield. The 40 mL vial already containing 1 (101 mg, 0.186 mmol) was stirred in 4 M HCl in dioxane (2 mL, 7.5 mmol) for 2.5 h. Reaction completion was confirmed with TLC and LCMS. The reaction was concentrated under reduced pressure and dried on vacuum pump overnight to give 2 (90 mg, 0.185 mmol) as an oil that was placed under N₂ and dissolved in sieve dried DCM (3 mL). Triethylamine (0.08 mL, 0.555 mmol), 2,3,5,6-tetrafluorophenol (34 mg, 0.203 mmol), and COMU (87 mg, 0.203 mmol) were added consecutively and the reaction was stirred for 3 h. Reaction completion was confirmed by LCMS. The reaction was diluted with DCM, washed with H₂O (×3), saturated NaHCO₃ (×3), brine, dried over MgSO₄, filtered, and concentrated under reduced pressure to give crude material that was dissolved in DCM (2.5 mL), loaded onto a 12 g silica gel column, and purified with flash chromatography (EtOAc/hexanes, 0-75%, product eluted at 50% EtAOc) to give LP-427-p (55 mg) as a colorless oil in 47% yield. LC/MS (ESI⁺) calculated m/z 633.37 (M), found 635.28 (M+H⁺).

### Synthesis of LP-428-p

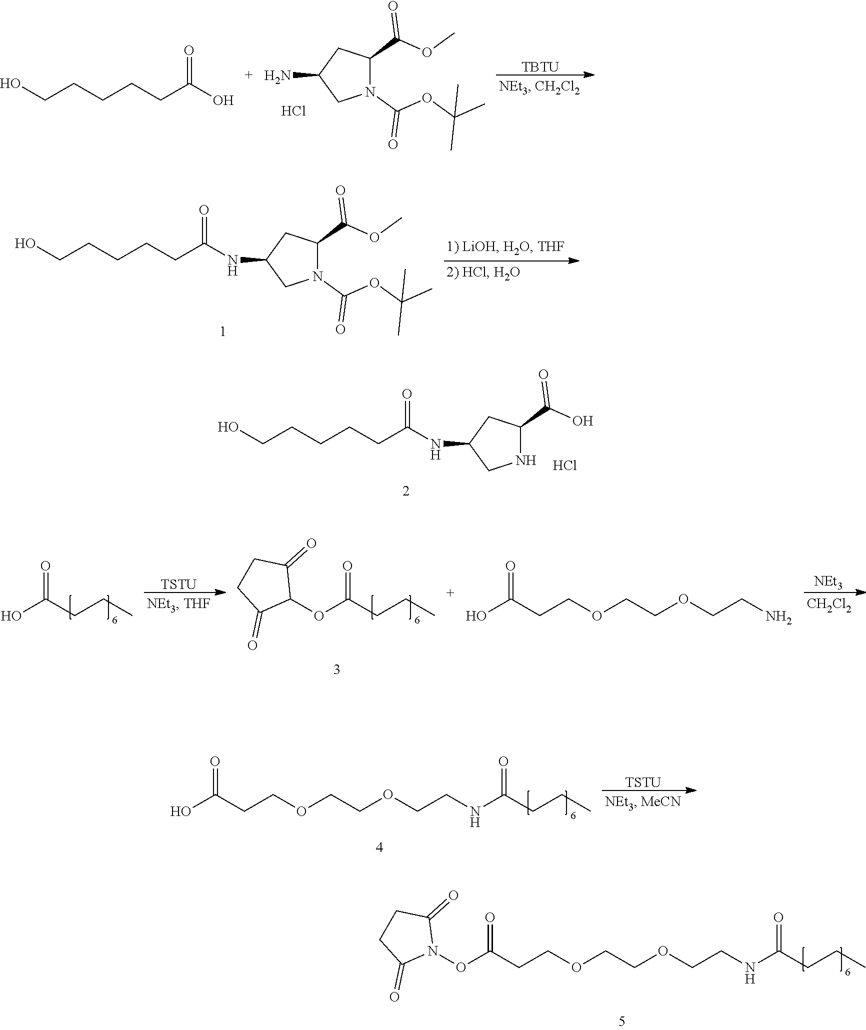

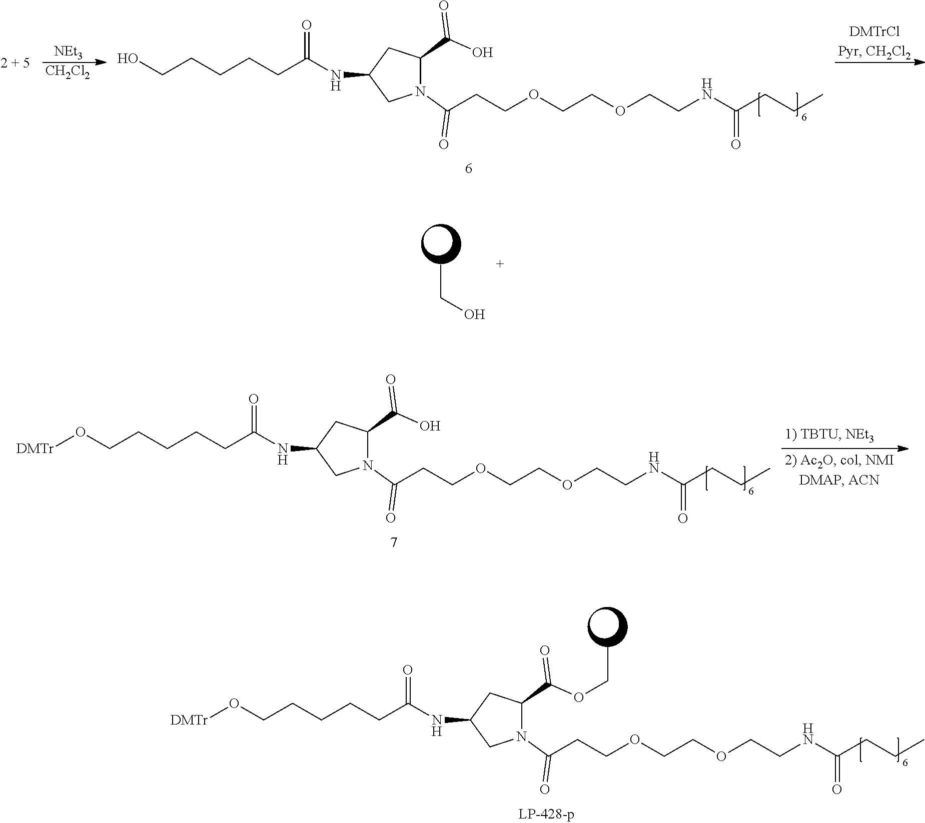

To a round bottom flask was added 6-hydroxyhexanoic acid (2.82 g, 21.3 mmol), DCM (28.3 mL), triethylamine (2.9 mL, 21.3 mmol), and TBTU (6.29 g, 19.5 mmol). The reaction was stirred for 1 min and then 2-amino-boc-proline-OMe HCl (5 g, 17.8 mmol) was added and the reaction was stirred until complete by HPLC (ELSD). The reaction was quenched with H₂O (30 mL). The layers were separated and the organic layer was washed with 1 M HCl (30 mL) and saturated NaHCO₃ (30 mL). A large emulsion formed that was then centrifuged. The layers were separated and the organic layer was dried over Na₂SO₄, filtered, and concentrated under reduced pressure to give a crude yellow oil that was loaded onto a 220 g silica gel column and purified with flash chromatography (MeOH/DCM, 0-10%) to give 1 (3.32 g) as a colorless oil in 52% yield. LC/MS calculated 358.21 (M), found 359.32 (M+H⁺). To a round bottom flask was added 1 (1.27 g, 3.56 mmol) and THF (7.5 mL). Aqueous LiOH (5 mL, 7.49 mmol) was added and the reaction was stirred for 1 h. HPLC with ELSD confirmed reaction completion. To the reaction was added 2 M HCl (21.6 mL, 43.1 mmol) and the reaction was stirred at 40° C. for 3 h. The reaction was concentrated under reduced pressure and dried on vacuum pump for 12 h to give 2 (1.4 g, HCl salt) as a thick sticky colorless oil that was used without purification.

To a round bottom flask was added myristic acid (8 g, 35.1 mmol), THF (40 mL), TSTU (11 g, 38.5 mmol), and triethylamine (5.8 mL, 42 mmol). The reaction was diluted with MeCN (40 mL) to give a solution that was stirred at room temperature for 17.5 h. The reaction was poured into H₂O (500 mL) and the crude precipitated product was filtered vacuum filtration, collected, suspended in MeCN, and concentrated under reduced pressure to give crude 3 (11.2 g) that was used without further purification. To a 100 mL round bottom flask was added amino-PEG₂-acid (2.4 g, 13.9 mmol), DCM (44 mL), and triethylamine (4.3 mL, 31.3 mmol). To this resulting suspension was added crude 3 (4.4 g, 13.6 mmol) over 4 min. The reaction was stirred for 19 h, slowly becoming homogeneous. Reaction completion was confirmed with HPLC with ELSD. The reaction was quenched with piperidine (0.188 mL), stirred for 30 min, and then diluted with H₂O (45 mL) and conc. HCl (3 mL). The layers were separated and the organic layer was washed with 0.5 M HCl (45 mL×2), H₂O (40 mL×2), dried over Na₂SO₄, filtered, and concentrated under reduced pressure to give 4 (4.9 g) as a white solid in 94% yield. LC/MS calculated m/z 387.30 (M), found 388.38 (M+H⁺) and 386.36 (M−H⁺). A portion of crude 4 (3 g, 7.88 mmol) was added to a round bottom flask and suspended in MeCN (25 mL) and triethylamine (1.3 mL, 9.5 mmol). TSTU (2.6 g, 8.7 mmol) and THF (15 mL) were added and the resulting yellow solution was stirred for 19 h. The reaction was partially concentrated under reduced pressure and then poured into H₂O (250 mL). A white precipitate formed that was filtered with vacuum filtration, suspended in MeCN, concentrated under reduced pressure, and then dried overnight to give 5 (3.6 g) as a white solid. ¹H NMR (400 MHz, CDCl₃) δ=6.02 (t, 1H), 3.85 (t, 2H), 3.66-3.60 (m, 2H), 3.57-3.52 (m, 2H), 3.47-3.41 (m, 2H), 2.89 (t, 2H), 2.83 (s, 4H), 2.16 (t, 2H), 1.66-1.58 (m, 2H), 1.34-1.22 (m, 22H), 0.877 (t, 3H).

To a round bottom flask was added 2 (1 g, 3.56 mmol), DMF (20 mL), and triethylamine (2.4 mL, 17.2 mmol). Intermediate 5 (1.3 g, 2.74 mmol) was added and the suspension was vigorously stirred for 16 h. HPLC and LCMS showed a mixture of 6 and side product in which both acid and amine of 2 reacted with 5. The reaction was diluted with H₂O (20 mL) and then 1 M NaOH (5 mL) and solid LiOH (98 mg) was added (pH 12) and the reaction was stirred for 2.5 h. The reaction was acidified with 2 M HCl (40 mL), diluted with H₂O (110 mL), and extracted with DCM (60 mL×3). The pooled organic layers were washed with 1 M HCl (75 mL×2), dried over Na₂SO₄, filtered, and concentrated under reduced pressure to give crude 6 (1.76 g) that was used without purification. To a round bottomed flask was added crude 6 (1.68 g, 2.74 mmol), DCM (17 mL), pyridine (17 mL), and DMT-Cl (1.02 g, 3.01 mmol). The orange solution was stirred for 17 h. LCMS confirmed reaction completion. The reaction was quenched with MeOH (5 mL), concentrated under reduced pressure, suspended in EtOAc (75 mL), and washed with H₂O (75 mL×2). An emulsion resulted that was resolved with the addition of brine (10 mL). The organic layer was dried over Na₂SO₄, filtered, and concentrated under reduced pressure to give crude 7 (3.19 g) that was dissolved in DCM (8 mL), loaded onto a 80 g silica gel column, and purified with flash chromatography (MeOH/DCM with 1% Et₃N; 0-10%) to give 7 (1.83 g) as a triethylammonium salt in 66% yield. LC/MS (ESI−) calculated m/z 915.56 (M), found 914.58 (M−H⁺).

To a round bottom flask was added 7 (1.84 g, 1.80 mmol) and MeCN (26 mL). Triethylamine (0.630 mL, 4.52 mmol), and TBTU (580 mg, 1.80 mmol) were added and the reaction was stirred for 2 min and then added to a reaction vessel containing NittoPhase HL native resin (hydroxy loading of 0.572 mmol/g). The reaction was shaken for 16 h, drained, and washed with MeCN (100 mL). To the resin was added NMI/MeCN (1:4, 12 mL), collidine/MeCN (3:2, 6 mL), Ac₂O/MeCN (2:3, 6 mL), and DMAP (92 mg). The capping reaction was shaken for 3 h, the resin was drained, washed with MeCN (150 mL), MeOH (100 mL), transferred to a plastic bottle, and dried on vacuum for 16 h. The resin loading was determined to be 0.371 mmol/g.

### Synthesis of LP-432-p

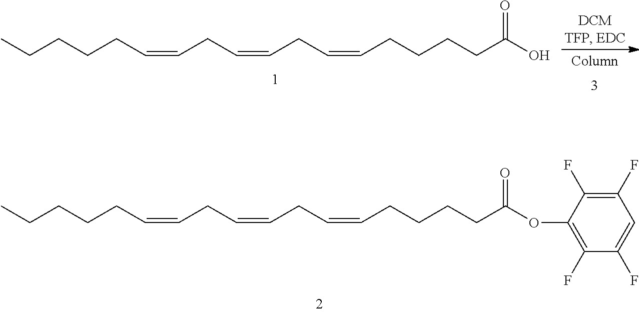 [SMILES: CCCCC/C=C\C/C=C\C/C=C\CCCCC(=O)O ; CCCCC/C=C\C/C=C\C/C=C\CCCCC(=O)Oc1c(F)c(F)cc(F)c1F]

Compound 1 (Gamma-linolenic acid, 150 mg) was dissolved in 5 mL DCM. Then tetrafluorophenol (Sigma #196789, 134 mg) was added, and the mixture was stirred for 10 minutes. Then EDC·-HCl (155 mg) was added and the mixture was stirred for 2 hours. The mixture was concentrated and the crude product was suspended in DCM (5 mL) and dry loaded in with silica onto a 12G column and purified with flash chromatography (EtOAc/hexanes, 0-30% over 35 minutes). Yield 197 mg. LC-MS: calculated [M+H] 426.50, found 427.66.

### Synthesis of LP-433-p

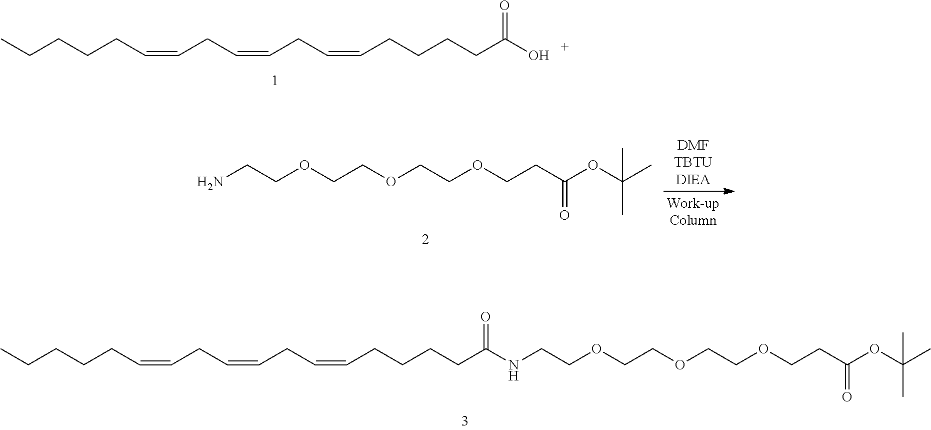 [SMILES: CCCCC/C=C\C/C=C\C/C=C\CCCCC(=O)O ; CC(C)(C)OC(=O)CCOCCOCCOCCN ; CCCCC/C=C\C/C=C\C/C=C\CCCCC(=O)NCCOCCOCCOCCC(=O)OC(C)(C)C]

Compound 1 (Sigma® #L2378, 250 mg) was dissolved in DMF then TBTU (331 mg) and DIPEA (0.636 mL) were added. The mixture was stirred for 10 minutes, then compound 2 (286 mg in DMF) was added. The mixture was stirred in the dark for 1 hour. Then the mixture was diluted with EtOAc (70 mL) washed with 3% citric acid in water (3×8 mL), H₂O (2×8 mL), and NaCl (1×8 mL), then dried over Na₂SO₄, filtered and concentrated on with rotary evaporator. The crude product was dissolved in 3 mL DCM, loaded onto a 24G column, and purified with flash chromatography (MeOH/DCM, 0-4% over 30 min). Yield 420 mg. LC-MS: calculated [M+H] 537.78, found 538.70.

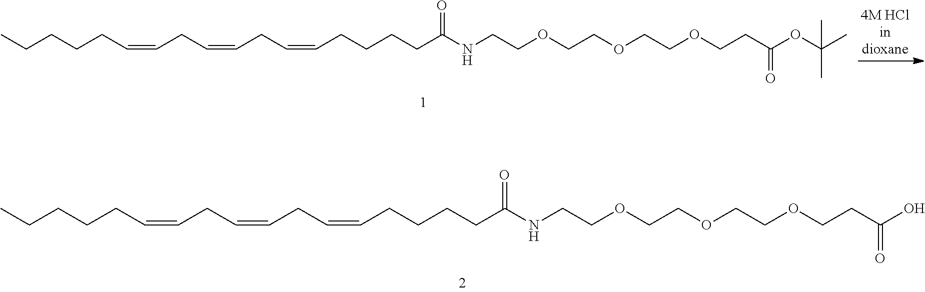

Compound 1 (400 mg) was dissolved in 6 mL 4M HCl in dioxane at 0° C. The mixture was stirred in the dark for 10 minutes, then allowed to warm to room temperature and stirred for 4 hours. The product was concentrated on rotary evaporator and placed under high vacuum. The product was dissolved and concentrated twice in DCM/toluene then placed on high vacuum overnight. Yield 358 mg. LC-MS: calculated [M+H] 481.67, found 482.37.

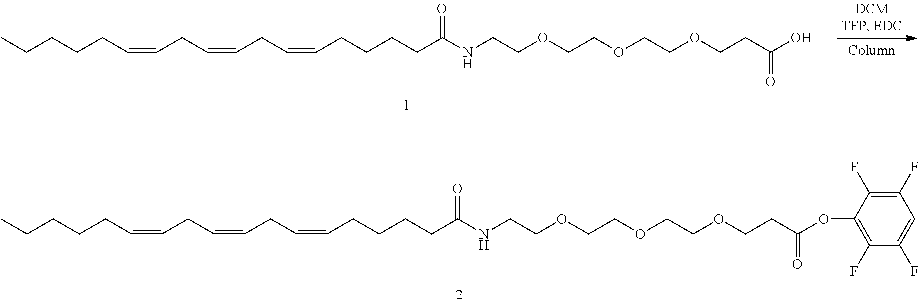 [SMILES: CCCCC/C=C\C/C=C\C/C=C\CCCCC(=O)NCCOCCOCCOCCC(=O)O ; CCCCC/C=C\C/C=C\C/C=C\CCCCC(=O)NCCOCCOCCOCCC(=O)Oc1c(F)c(F)cc(F)c1F]

Compound 1 (358 mg) was dissolved in 7.5 mL DCM. Then tetrafluorophenol (185 mg) was added. The mixture was allowed to stir for 10 minutes then EDC-HCl (214 mg) was added. The mixture was allowed to stir for 2 hours. Then the crude reaction was concentrated in vacuo, suspended in DCM, dry loaded with silica onto a 12G column, and purified with flash chromatography (EtOAc/hexanes, 0-100% over 35 min.) Yield 373 mg. LC-MS: calculated [M+H] 629.73, found 630.71.

### Synthesis of LP-444-p

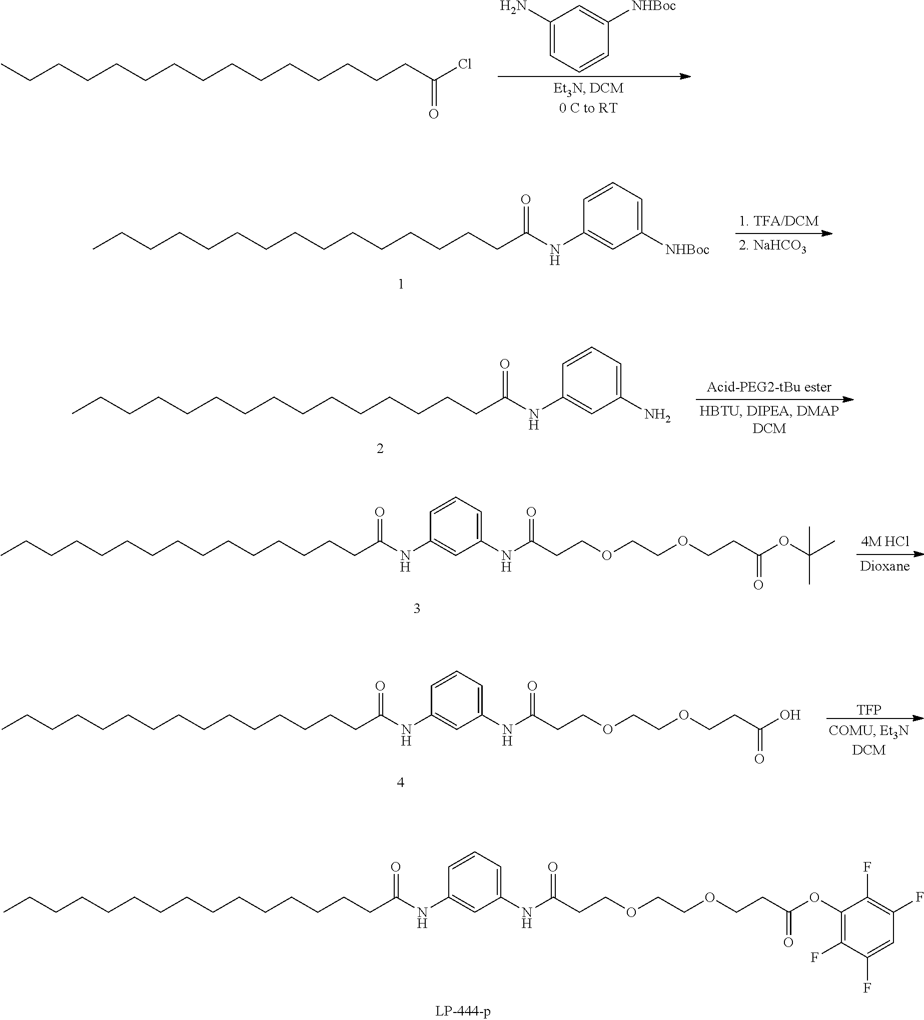

To a 100 mL round bottom flask with stir bar and under N₂ was added tert-butyl (3-aminophenyl)carbamate (500 mg, 2.4 mmol), sieve dried DCM (25 mL), and triethylamine (1 mL, 7.2 mmol). The solution was cooled in an ice bath for 10 min and then palmitoyl chloride (0.8 mL, 2.6 mmol) was added dropwise over 1 min. The reaction was stirred on ice for 15 min and then the ice bath was removed and the reaction was stirred for 18 h, warming to room temperature. Reaction completion was confirmed with TLC (ninhydrin staining). The reaction was quenched with 7 N NH₃ in MeOH (0.5 mL) and stirred for an additional 10 min. The reaction was vacuum filtered, concentrated under reduced pressure, and then dried on vacuum pump for 2 h to give 1 (1.07 g) that was used without purification. The round bottom flask containing 1 (1.07 g, 2.39 mmol) was placed under N₂ and cooled in an ice bath for 10 min. Dry DCM (15 mL) and TFA (10 mL) were added consecutively, the reaction was stirred on ice for 10 min, and then the ice bath was removed and the reaction was stirred for 2 h, warming to room temperature. Reaction completion was confirmed by LCMS. The reaction was concentrated under reduced pressure to give crude 2 that was dissolved in DCM (50 mL) and concentrated again. The reaction was dissolved in DCM and washed with Na₂CO₃ (10% aqueous solution, ×3 washes), brine, dried over MgSO₄, filtered, and concentrated under reduced pressure to give 2 (853 mg, free base) as a grey solid that was carried forward without further purification. To a 40 mL vial with stir bar and under N₂ was added 2 (273 mg, 0.784 mmol), sieve dried DCM (15 mL), DIPEA (0.54 mL, 3.13 mmol), and acid-PEG₂-tert-butyl ester (239 mg, 0.862 mmol). HBTU (327 mg, 0.862 mmol) and DMAP (10 mol %, 10 mg) were added and the reaction was stirred for 4 h. LCMS confirmed that the reaction was ˜90% complete. The reaction was diluted with DCM and washed with H₂O (×2), saturated NaHCO₃ (×4), brine, dried over MgSO₄, filtered, and concentrated under reduced pressure to give crude material that was dissolved in DCM (5 mL), loaded onto 24 g silica gel column, and purified with flash chromatography (MeOH/DCM 0-5%) to give 3 (309 mg) as a tan solid in 67% yield. LC/MS (ESI⁺) calculated m/z 590.43 (M), found 591.83 (M+H⁺).

To a 40 mL vial with stir bar was added 3 (309 mg, 0.522 mmol) and 4M HCl in dioxane (6.5 mL, 26.1 mmol). The reaction was tightly capped and stirred for 2.5 h. Reaction completion was confirmed by LCMS. The reaction was concentrated under reduced pressure and dried on vacuum pump for 16 h to give 4 that was carried forward without purification. Crude 4 (280 mg, 0.523 mmol), in a 40 mL vial with stir bar and under N₂, was dissolved in sieve dried DCM (10 mL). 2,3,5,6-Tetrafluorophenol (96 mg, 0.575 mmol) and triethylamine (0.22 mL, 1.56 mmol) were added and the resulting solution was cooled in an ice bath for 15 min. COMU (246 mg, 0.575 mmol) was added slowly and the reaction was stirred on ice for 10 min and then the ice bath was removed and the reaction was stirred for 3 h. Reaction completion was confirmed by LCMS. The reaction was diluted with DCM and washed with H₂O (×4), brine, dried over MgSO₄, filtered, and concentrated under reduced pressure to give crude material that was dissolved in DCM (3.5 mL), loaded onto a 12 g silica gel column, and purified with flash chromatography (EtOAc/hexanes, 0-100% over 40 min, product eluted at 45% EtOAc) to give LP-444-p (178 mg) as white solid in 50% yield. LC/MS (ESI⁺) calculated m/z 682.36 (M), found 683.99 (M+H⁺)

### Synthesis of LP-445-p

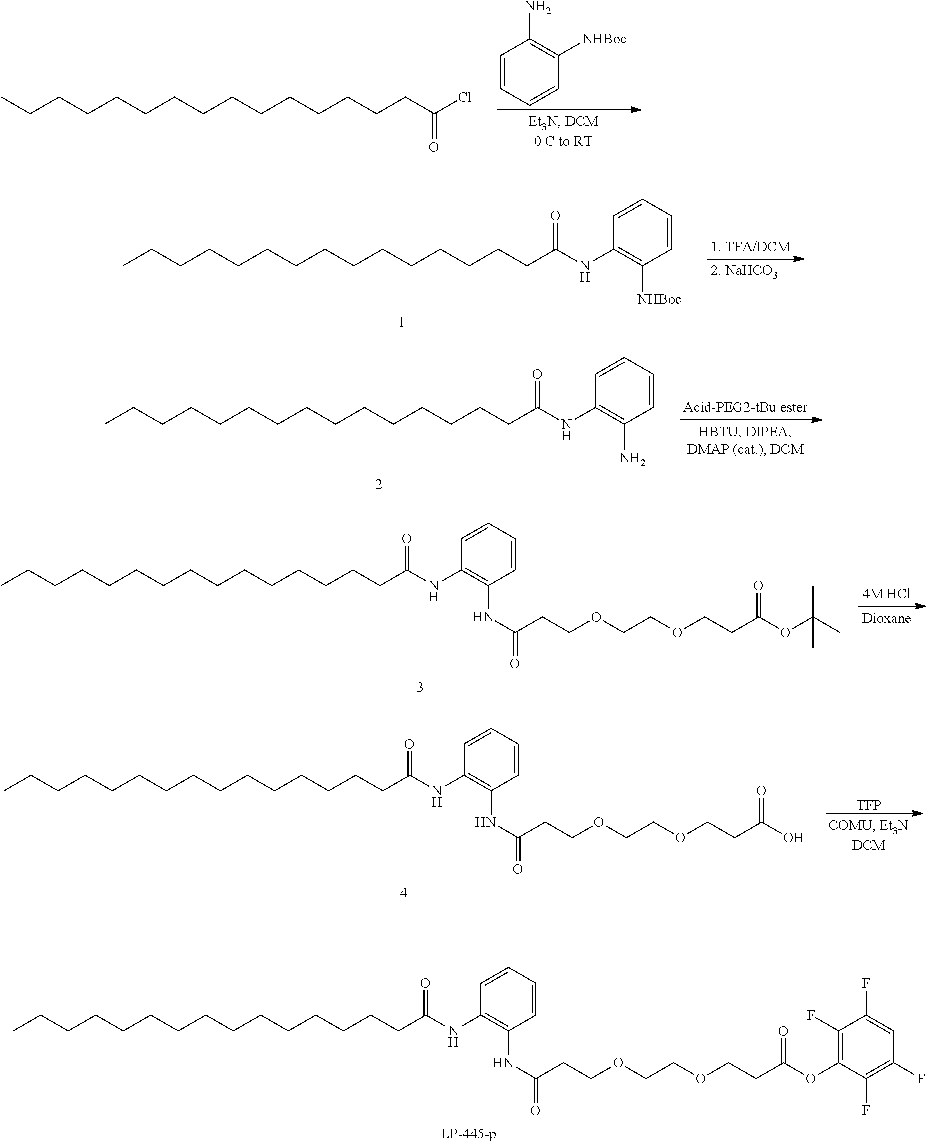

To a 100 mL round bottom flask with stir bar and under N₂ was added tert-butyl (2-aminophenyl)carbamate (500 mg, 2.4 mmol), sieve dried DCM (25 mL), and triethylamine (1 mL, 7.2 mmol). The solution was cooled in an ice bath for 10 min and then palmitoyl chloride (0.8 mL, 2.6 mmol) was added dropwise over 1 min. The reaction was stirred on ice for 15 min and then the ice bath was removed and the reaction was stirred for 1 h, warming to room temperature. Reaction completion was confirmed with TLC (ninhydrin staining). The reaction was quenched with 7 N NH₃ in MeOH (0.5 mL) and stirred for an additional 20 min. The reaction was concentrated under reduced pressure and then dried on vacuum pump for 2 h to give 1 (1.07 g) that was used without purification. The round bottom flask containing 1 (1.07 g, 2.39 mmol) was placed under N₂ and cooled in an ice bath for 10 min. Dry DCM (15 mL) and TFA (10 mL) were added consecutively, the reaction was stirred on ice for 10 min, and then the ice bath was removed and the reaction was stirred for 1.5 h, warming to room temperature. Reaction completion was confirmed by LCMS. The reaction was concentrated under reduced pressure to give crude 2 which was then suspended in DCM (50 mL), concentrated again, and dried on vacuum pump for 16 h. The crude material was suspended in DCM and washed with Na₂CO₃ (10% aqueous solution, ×4 washes), brine, dried over MgSO₄, filtered, and concentrated under reduced pressure to give 2 (807 mg, free base) as a grey solid that was carried forward without further purification. To a 40 mL vial with stir bar and under N₂ was added 2 (187 mg, 0.539 mmol), sieve dried DCM (12 mL), DIPEA (0.375 mL, 2.16 mmol), acid-PEG2-tert-butyl ester (165 mg, 0.593 mmol) and DMAP (10 mol %, 6.5 mg, 0.054 mmol). The reaction was cooled in an ice bath for 10 min and then HBTU (226 mg, 0.593 mmol) was slowly added and the reaction was stirred for 10 min on ice. The ice bath was removed and the reaction was stirred for 18 h. LCMS confirmed reaction completion. The reaction was diluted with DCM and washed with H₂O (×2), saturated NaHCO₃ (×3), brine, dried over MgSO₄, filtered, and concentrated under reduced pressure to give crude material that was dissolved in DCM (5 mL), loaded onto a 24 g silica gel column, and purified with flash chromatography (MeOH/DCM 0-8% over 60 min) to give 3 (232 mg, oil solid) as a mixture of 3 and tetramethylurea by-product. LC/MS (ESI⁺) calculated m/z 590.43 (M), found 591.83 (M+H⁺).

To a 40 mL vial with stir bar was added 3 (232 mg, 0.392 mmol) and 4M HCl in dioxane (4.5 mL, 19.6 mmol). The reaction was tightly capped and stirred for 3 h. Reaction completion was confirmed by LCMS. The reaction was concentrated under reduced pressure and dried on vacuum pump for 16 h to give 4 that was carried forward without purification. Crude 4 (210 mg, 0.393 mmol), in a 40 mL vial with stir bar and under N₂, was dissolved in sieve dried DCM (10 mL). 2,3,5,6-Tetrafluorophenol (72 mg, 0.432 mmol) and triethylamine (0.16 mL, 1.18 mmol) were added and the resulting solution was cooled in an ice bath for 15 min. COMU (185 mg, 0.432 mmol) was added slowly and the reaction was stirred on ice for 10 min and then the ice bath was removed and the reaction was stirred for 3 h. Reaction completion was confirmed by LCMS. The reaction was diluted with DCM and washed with H₂O (×3), brine, dried over MgSO₄, filtered, and concentrated under reduced pressure to give crude material that was dissolved in DCM (4 mL), loaded onto a 12 g silica gel column, and purified with flash chromatography (EtOAc/hexanes, 0-75% over 50 min, product eluted at 35% EtOAc) to give LP-445-p (157 mg) as white solid in 59% yield. LC/MS (ESI⁺) calculated m/z 682.36 (M), found 684.08 (M+H⁺).

### Synthesis of LP-446-p

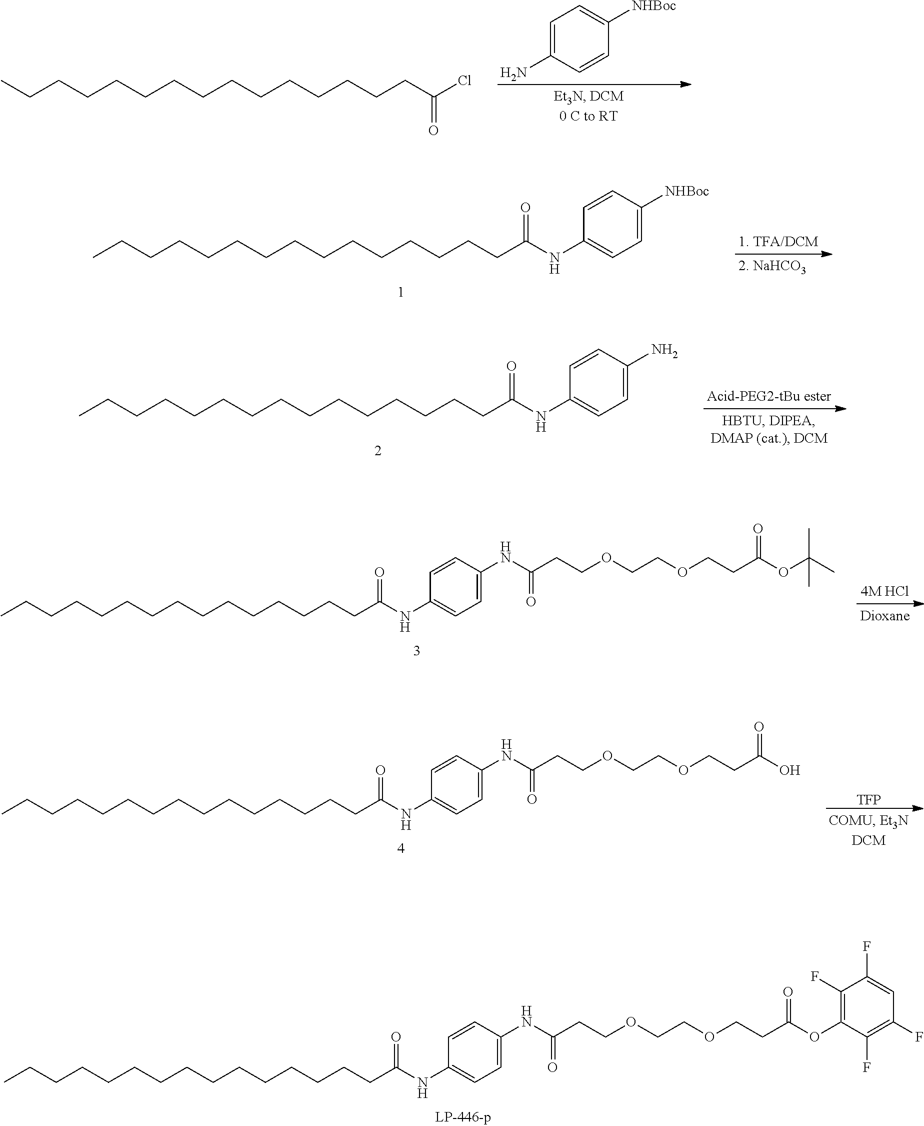

To a 100 mL round bottom flask with stir bar and under N₂ was added tert-butyl (4-aminophenyl)carbamate (250 mg, 1.2 mmol), sieve dried DCM (12 mL), and triethylamine (0.5 mL, 3.6 mmol). The solution was cooled in an ice bath for 10 min and then palmitoyl chloride (0.4 mL, 1.3 mmol) was added dropwise over 1 min. The reaction was stirred on ice for 15 min and then the ice bath was removed and the reaction was stirred for 16 h, warming to room temperature. Reaction remained heterogeneous. Reaction completion was confirmed with TLC (sample aliquot diluted with MeOH to make more homogeneous prior to spotting on plate, ninhydrin staining used for visualization). MeOH (25 mL) was added to quench the reaction and the reaction was stirred for 1 h. The reaction was concentrated under reduced pressure and then dried on vacuum pump for 2 h to give 1 (535 mg) that was used without purification. The round bottom flask containing 1 (535 mg, 1.2 mmol) was placed under N₂ and cooled in an ice bath for 10 min. Dry DCM (5 mL) and TFA (5 mL) were added consecutively, the reaction was stirred on ice for 10 min, and then the ice bath was removed and the reaction was stirred for 2 h, warming to room temperature. The reaction started heterogenous and slowly became homogeneous. Reaction completion was confirmed by LCMS. The reaction was concentrated under reduced pressure to give crude 2 which was dissolved in DCM (50 mL) concentrated again, and then suspended in PhMe (15 mL), concentrated, and dried on vacuum pump for 16 h. The reaction was dissolved in DCM and washed with Na₂CO₃ (10% aqueous solution, ×3 washes), brine, dried over MgSO₄, filtered, and concentrated under reduced pressure to give 2 (395 mg, free base) as a grey solid that was carried forward without further purification. To a 40 mL vial with stir bar and under N₂ was added 2 (250 mg, 0.721 mmol), sieve dried DCM (15 mL), DIPEA (0.5 mL, 2.88 mmol), acid-PEG₂-tert-butyl ester (220 mg, 0.793 mmol) and DMAP (10 mol %, 9 mg, 0.0721 mmol). The resulting suspension was cooled in an ice bath for 10 min and then HBTU (301 mg, 0.793 mmol) was slowly added and the reaction was stirred for 10 min on ice. The ice bath was removed and the reaction was stirred for 18 h. The reaction became homogeneous after 1 h. Reaction completion was confirmed by LCMS. The reaction was diluted with DCM and washed with H₂O (×2), saturated NaHCO₃ (×5; minor emulsions observed), brine, dried over MgSO₄, filtered, and concentrated under reduced pressure to give crude material that was dissolved in DCM (7 mL, sonication required), loaded onto a 24 g silica gel column, and purified with flash chromatography (MeOH/DCM 0-3% over 40 min) to give 3 (163 mg) as a white solid in 38% yield. LC/MS (ESI⁺) calculated m/z 590.43 (M), found 591.83 (M+H⁺).

To a 40 mL vial with stir bar was added 3 (163 mg, 0.275 mmol) and 4M HCl in dioxane (4 mL, 13.8 mmol). The reaction was tightly capped and stirred for 3 h. The reaction remained a heterogeneous suspension. Reaction completion was confirmed by LCMS. The reaction was concentrated under reduced pressure and dried on vacuum pump for 16 h to give 4 that was carried forward without purification. Crude 4 (147 mg, 0.274 mmol), in a 40 mL vial with stir bar and under N₂, was dissolved in sieve dried DCM (10 mL). To the suspension was added 2,3,5,6-tetrafluorophenol (50 mg, 0.302 mmol), triethylamine (0.11 mL, 0.822 mmol), and COMU (129 mg, 0.302 mmol) consecutively and the reaction was stirred for 2 h. Reaction remained heterogeneous. Reaction completion was confirmed by LCMS. The reaction was diluted with DCM and washed with H₂O (×3), brine, dried over MgSO₄, filtered, and concentrated under reduced pressure to give crude material that was dissolved in DCM (20 mL) and dry loaded with silica onto a 12 g silica gel column and purified with flash chromatography (EtOAc/hexanes, 0-75%, product eluted at 55% EtOAc) to give LP-446-p (39 mg) as white solid in 20% yield. LC/MS (ESI⁺) calculated m/z 682.36 (M), found 684.08 (M+H⁺).

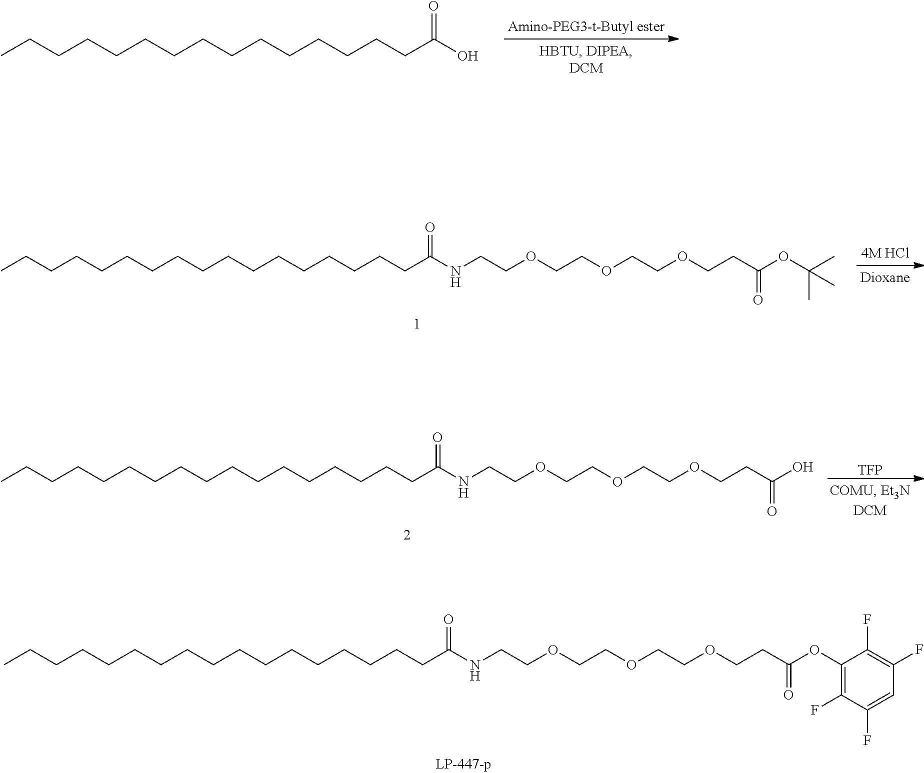

To a 40 mL vial with stir bar and under N₂ was added steric acid (100 mg, 0.352 mmol) and sieve dried DCM (3 mL). DIPEA (0.18 mL, 1.06 mmol), amine-PEG₃-tert-butyl ester (117 mg, 0.421 mmol), and HBTU (162 mg, 0.427 mmol) were added consecutively and the reaction was stirred for 3 h. Reaction completion was confirmed by TLC (b-cresol green staining). The reaction was diluted with DCM and washed with H₂O, saturated NaHCO₃ (×3), brine, dried over MgSO₄, filtered, and concentrated under reduced pressure to give crude product that was dissolved in DCM (3 mL), loaded onto a 12 g silica gel column, and purified with flash chromatography (EtOAc/hexanes 0-75%; product eluted at 50% EtOAc) to give 1 (140 mg) as a white solid in 74% yield. The 40 mL vial already containing 1 (140 mg, 0.257 mmol) was stirred in 4 M HCl in dioxane (3 mL) for 2.5 h. Reaction completion was confirmed with LCMS. The reaction was concentrated under reduced pressure and dried on vacuum pump for 16 h to give 2 (125 mg, 0.256 mmol) as a white solid that was placed under N₂ and dissolved in sieve dried DCM (3 mL). Triethylamine (0.1 mL, 0.768 mmol), 2,3,5,6-tetrafluorophenol (47 mg, 0.281 mmol), and COMU (120 mg, 0.281 mmol) were added consecutively and the reaction was stirred for 2.5 h. Reaction completion was confirmed by LCMS. The reaction was diluted with DCM, washed with H₂O (×3), brine, dried over MgSO₄, filtered, and concentrated under reduced pressure to give crude material that was dissolved in DCM (2.5 mL), loaded onto a 12 g silica gel column, and purified with flash chromatography (EtOAc/hexanes, 0-75% over 40 min, product eluted at 50% EtOAc) to give LP-447-p (90 mg) as a colorless oil in 56% yield. LC/MS (ESI⁺) calculated m/z 635.38 (M), found 636.56 (M+H⁺).

### Synthesis of LP-453-p

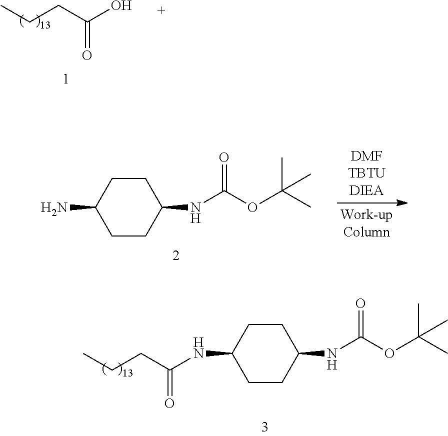

Compound 1 (palmitic acid, 400 mg) was dissolved in 9 mL DMF. Then TBTU (551 mg) and DIPEA (1.1 mL) were added and the mixture was stirred for 10 minutes. Then compound 2 (368 mg in DMF) was added and the mixture was stirred for 1 hour. Then the mixture was diluted with 125 mL EtOAc and washed with 3% citric acid in water (3×10 mL), H₂O (2×10 mL), and NaCl (1×10 mL), dried over Na₂SO₄, filtered and concentrated on rotary evaporator and placed under high vacuum. The crude product was loaded onto a 24G column in 3 mL DCM and purified with flash chromatography (MeOH/DCM, 0-5% over 30 min.) Yield 555 mg. LC-MS: calculated [M+H] 452.72, found 453.85.

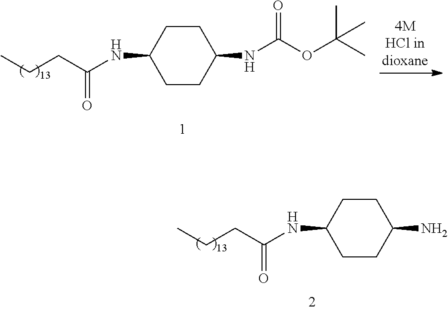

Compound 1 (555 mg) was dissolved in 8 mL 4M HCl in dioxane. The mixture was stirred for 2 hours. The product was concentrated on rotary evaporator, placed under high vacuum, then redissolved and concentrated twice in DCM/toluene, and placed on high vacuum overnight. Yield 432 mg. LC-MS: calculated [M+H] 352.61, found 353.49.

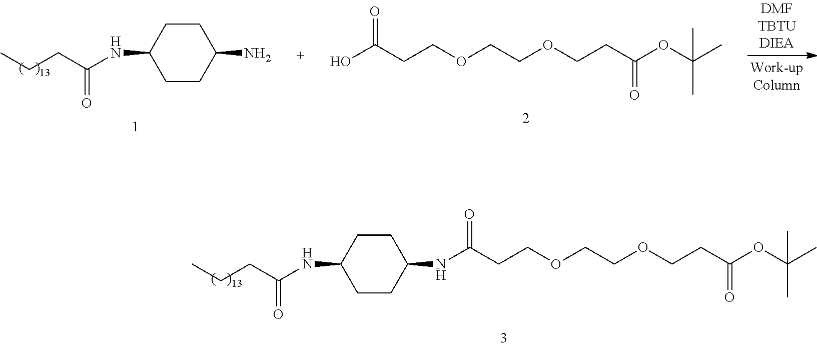

Compound 1 (432 mg) was dissolved in 10 mL DMF. Then TBTU (452 mg) and DIPEA (0.867 mL) were added and the mixture was stirred for 10 minutes. Then compound 2 (370 mg in DMF) was added and the mixture was stirred for 1 hour. The mixture was diluted with 150 mL EtOAc, washed with 3% citric acid in water (3×13 mL), H₂O (2×13 mL), NaHCO₃ (2×13 mL), and NaCl (1×13 mL), then dried over Na₂SO₄, filtered and concentrated with rotary evaporator. The crude product was dissolved in 3 mL DCM, loaded onto a 24G column, and purified with flash chromatography (MeOH/DCM, 0-4% over 30 min). Yield 390 mg. LC-MS: calculated [M+H] 596.89, found 597.86.

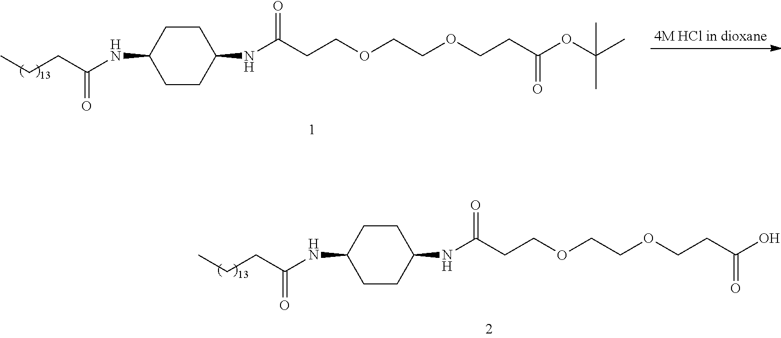

Compound 1 (400 mg) was dissolved in 6 mL 4M HCl in dioxane. The mixture was stirred for 2 hours. Then the product was concentrated on rotary evaporator and placed under high vacuum. The product was redissolved and concentrated twice in DCM/toluene and placed on high vacuum overnight. Yield 390 mg. LC-MS: calculated [M+H] 540.79, found 541.79.

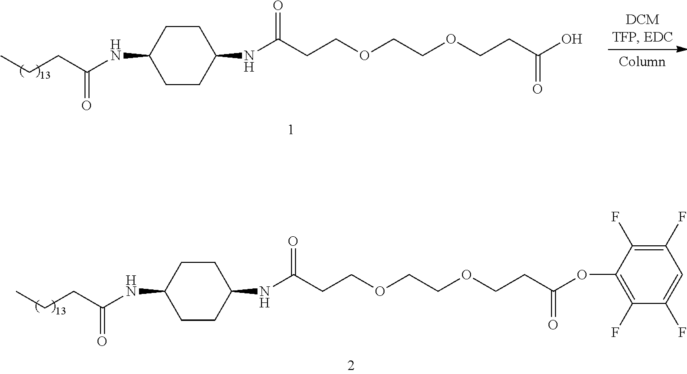

Compound 1 (362 mg) was dissolved in 7 mL DCM. Then tetrafluorophenol (167 mg) was added and the mixture was stirred for 10 minutes. Then EDC HCl (193 mg) was added. The mixture was stirred for 2 hours. The crude reaction was concentrated, suspended in DCM, and dry loaded with silica onto a 12G column and purified with flash chromatography (EtOAc/hexanes, 0-100% over 40 min.) Yield 127 mg. LC-MS: calculated [M+H] 688.85, found 689.93.

### Synthesis of LP-455-p

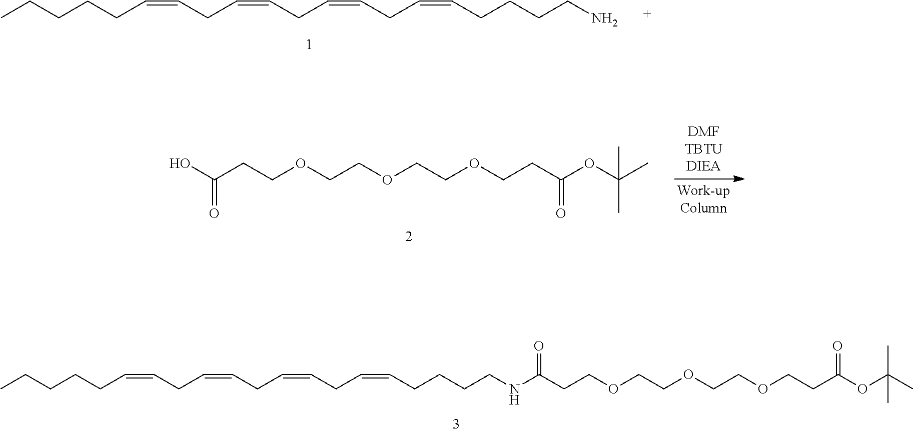 [SMILES: CCCCC/C=C\C/C=C\C/C=C\C/C=C\CCCCN ; CC(C)(C)OC(=O)CCOCCOCCOCCC(=O)O ; CCCCC/C=C\C/C=C\C/C=C\C/C=C\CCCCNC(=O)CCOCCOCCOCCC(=O)OC(C)(C)C]

Compound 2 (61 mg) was dissolved in 2 mL DMF then TBTU (64 mg) and DIPEA (0.122 mL) were added and the mixture was stirred for 10 minutes. Then compound 1 (50 mg in DMF) was added. The flask was covered in foil and the mixture was stirred for 1 hour. The mixture was then diluted with EtOAc (25 mL) and washed with 3% citric acid in water (3×3 mL), H₂O (2×3 mL), sodium bicarbonate (1×3 mL), and NaCl (1×3 mL), then dried over Na₂SO₄, filtered and concentrated with rotary evaporator. The crude product was brought up in 1 mL DCM, loaded onto a 4G column, and purified with flash chromatography (MeOH/DCM, 0-4% over 30 min. Yield 64 mg. LC-MS: calculated [M+H] 577.85, found 578.87.

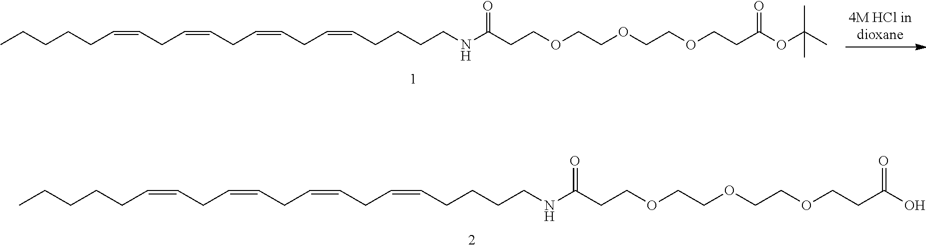

Compound 1 (64 mg) was dissolved in 2 mL 4M HCl in dioxane. The mixture was stirred for 2 hours in the dark. The product was concentrated on rotary evaporator and placed under high vacuum, then dissolved and concentrated twice with DCM/toluene and placed on high vacuum overnight. Yield 59 mg. LC-MS: calculated [M+H] 521.74, found 522.71.

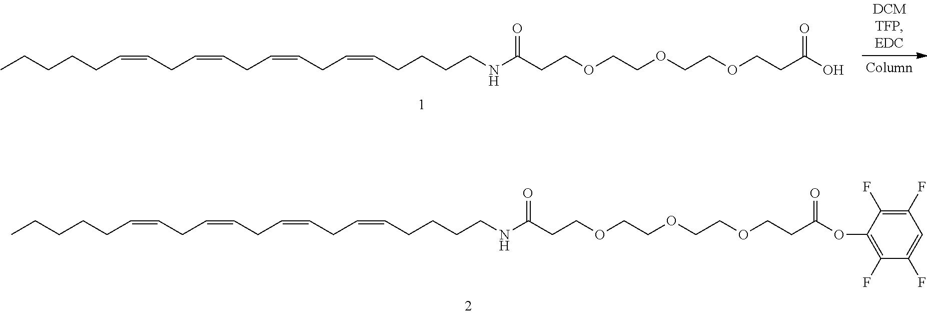 [SMILES: CCCCC/C=C\C/C=C\C/C=C\C/C=C\CCCCNC(=O)CCOCCOCCOCCC(=O)O ; CCCCC/C=C\C/C=C\C/C=C\C/C=C\CCCCNC(=O)CCOCCOCCOCCC(=O)Oc1c(F)c(F)cc(F)c1F]

Compound 1 (57 mg) was dissolved in 3 mL DCM. Then tetrafluorophenol (27 mg) was added and the mixture was stirred for 10 minutes. Then EDC HCl (31 mg) was added and the mixture was stirred for 2 hours. The reaction mixture was concentrated, dissolved in 4 mL DCM, and dry loaded with silica onto a 4G column and purified with flash chromatography (EtOAc/hexanes, 0-100% over 35 min.) Yield 59 mg. LC-MS: calculated [M+H] 669.80, found 670.71.

### Synthesis of LP-457-p

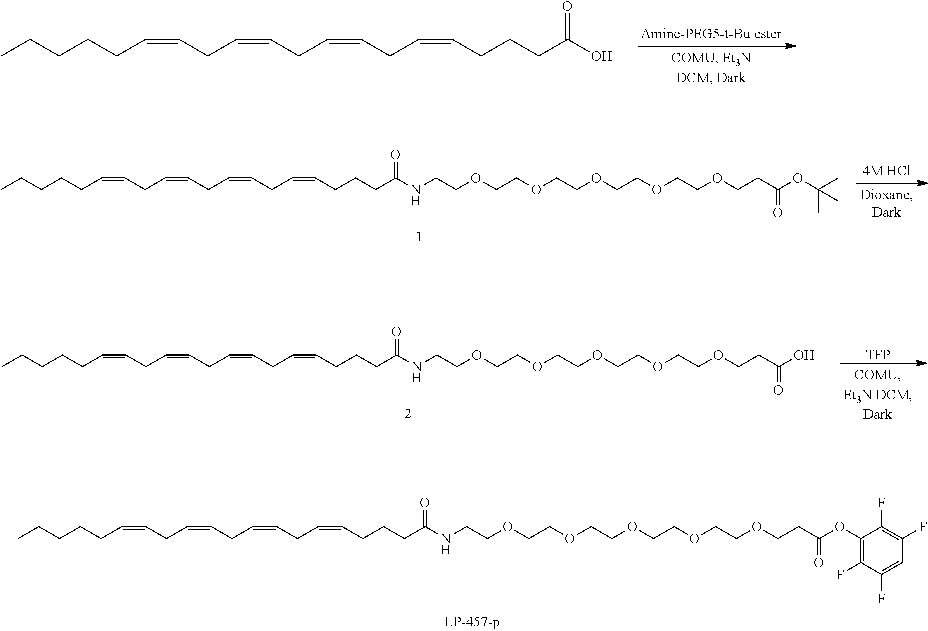

To a 40 mL vial with stir bar and under N₂ was added arachidonic acid (142 mg, 0.466 mmol) and sieve dried DCM (10 mL). Triethylamine (0.32 mL, 2.33 mmol), amine-PEG₅-tert-butyl ester (187 mg, 0.513 mmol), and COMU (219 mg, 0.513 mmol) were added consecutively and the reaction was stirred for 2 h wrapped in foil. Reaction completion was confirmed by TLc (KMnO₄ staining). The reaction was diluted with DCM and washed with H₂O (×3), saturated NaHCO₃ (×2), brine, dried over MgSO₄, filtered, and concentrated under reduced pressure (shielding product from light) to give crude product that was dissolved in DCM (3 mL), loaded onto a 12 g silica gel column, and purified with flash chromatography (EtOAc/hexanes 0-100% over 20 min; product eluted at 80% EtOAc) to give 1 (172 mg) as a white solid in 57% yield. The 40 mL vial already containing 1 (172 mg, 0.264 mmol) was wrapped in foil and stirred in 4 M HCl in dioxane (5 mL) for 3 h. Reaction completion was confirmed with LCMS. The reaction was concentrated under reduced pressure (shielding product from light) and dried on vacuum pump (shielding sample from light) for 16 h to give 2 (157 mg, 0.264 mmol) as a white solid that was placed under N₂ and dissolved in sieve dried DCM (10 mL). Triethylamine (0.18 mL, 1.32 mmol) and 2,3,5,6-tetrafluorophenol (48 mg, 0.289 mmol) were added and the reaction was cooled in an ice bath for 10 min. COMU (124 mg, 0.289 mmol) was added and the reaction was stirred for 15 min on ice. The ice bath was then removed, the reaction was wrapped in foil, and stirred for 2 h warming to room temperature. Reaction completion was confirmed by LCMS. The reaction was diluted with DCM, washed with H₂O (×3, large emulsions formed that cleared after 20 min), brine, dried over MgSO₄, filtered, and concentrated under reduced pressure (shielding product from light) to give crude material that was dissolved in DCM (3 mL), loaded onto a 12 g silica gel column, and purified with flash chromatography (EtOAc/hexanes, 0-100% over 30 min, product eluted at 80% EtOAc) to give LP-457-p (56 mg) as a yellow oil in 29% yield. LC/MS (ESI⁺) calculated m/z 743.40 (M), found 745.20 (M+H⁺).

### Synthesis of LP-458-p

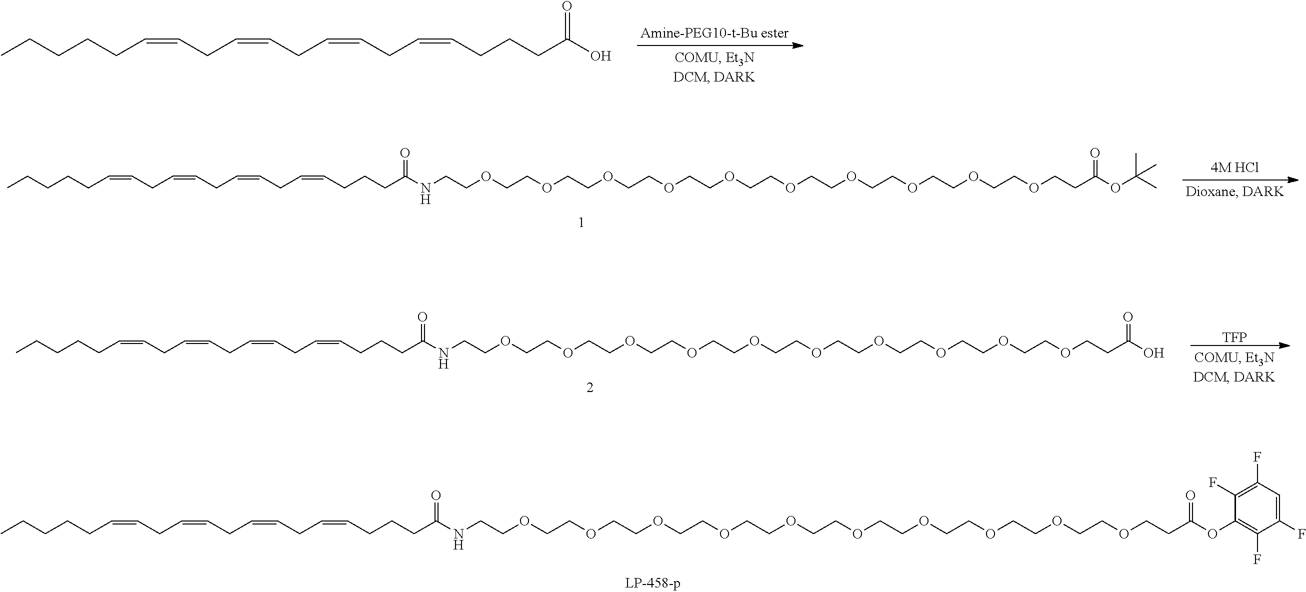

To a 40 mL vial with stir bar and under N₂ was added arachidonic acid (150 mg, 0.492 mmol) and sieve dried DCM (10 mL). Triethylamine (0.34 mL, 2.46 mmol), amine-PEG10-tert-butyl ester (317 mg, 0.541 mmol), and COMU (232 mg, 0.541 mmol) were added consecutively and the reaction was stirred for 5 h wrapped in foil. Reaction completion was confirmed by TLC (b-cresol green and KMnO₄ staining). The reaction was concentrated down to 5 mL (shielding reaction from light), loaded onto a 24 g silica gel column, and purified with flash chromatography (MeOH/DCM 0-4% over 40 min; product eluted at 2% MeOH) to give 1 (254 mg) as a colorless oil in 60% yield. The 40 mL vial already containing 1 (254 mg, 0.291 mmol) was wrapped in foil and stirred in 4 M HCl in dioxane (7 mL) for 3 h. Reaction completion was confirmed with LCMS. The reaction was concentrated under reduced pressure (shielding product from light) and dried on vacuum pump for 16 h to give 2 (237 mg, 0.29 mmol) as a white solid that was placed under N₂ and dissolved in sieve dried DCM (8 mL). Triethylamine (0.16 mL, 1.16 mmol), 2,3,5,6-tetrafluorophenol (58 mg, 0.348 mmol), and COMU (150 mg, 0.348 mmol) was added and the reaction was wrapped in foil and stirred for 2 h. The reaction was not complete after 2 h, thus additional 2,3,5,6-tetrafluorophenol (0.5 equiv.) and COMU (0.5 equiv.) were added and the reaction was stirred for an additional 1 h. Reaction completion was confirmed by LCMS. The reaction was concentrated under reduced pressure (shielding product from light) to give crude material that was dissolved in DCM (3 mL), loaded onto a 12 g silica gel column, and purified with flash chromatography (MeOH/DCM, 0-4% over 50 min, product eluted at 3% MeOH) to give LP-458-p (63 mg) as an oil in 23% yield. LC/MS (ESI⁺) calculated m/z 963.53 (M), found 965.34 (M+H⁺).

### Synthesis of LP-459-p

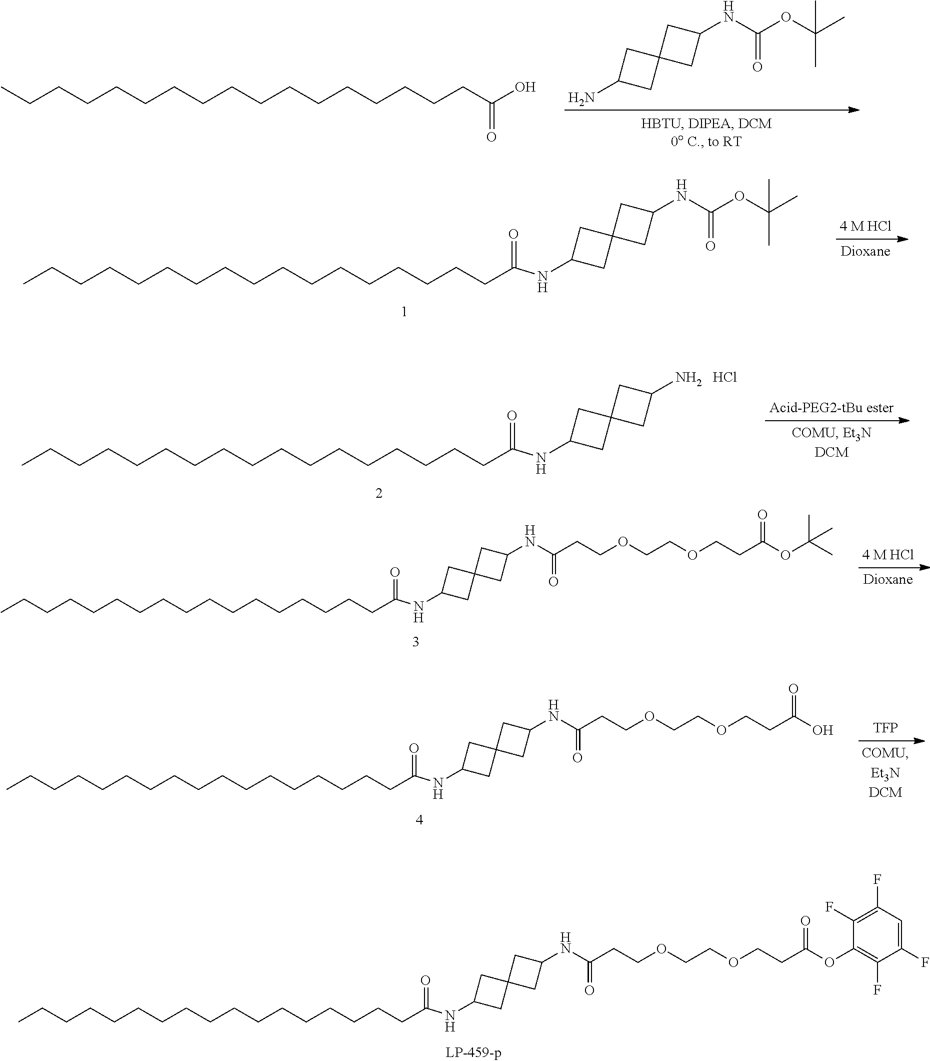

To a 40 mL vial with stir bar and under N₂ was added stearic acid (300 mg, 1.05 mmol), sieve dried DCM (15 mL), DIPEA (0.73 mL, 4.2 mmol), and HBTU (441 mg, 1.16 mmol). The reaction was stirred for 2 min and then N-(2-aminospiro[3.3]hept-6-yl)carbamic acid tert-butyl ester (262 mg, 1.16 mmol) was added and the reaction was stirred for 1.5 h. TLC (b-cresol green staining) confirmed reaction completion. The reaction was diluted with DCM (50 mL) and washed with saturated NaHCO₃ (×4) and brine. The organic phase remained heterogeneous while the aqueous phase was homogeneous during work-up. After washings, the organic layer was diluted with MeOH until homogeneous, dried over MgSO₄, filtered, and concentrated under reduced pressure to give crude material that was dissolved in DCM/MeOH (5:1), dry loaded with silica gel onto a 24 g silica gel column, and purified with flash chromatography (MeOH/DCM, 0-15% over 60 min, ELS detection, product eluted at 2.5% MeOH) to give 1 (227 mg) as a white solid in 44% yield. LC/MS (ESI⁺) calculated m/z 492.43 (M), found 493.90 (M+H⁺).

To the 40 mL vial containing 1 (227 mg, 0.461 mmol) was added stir bar and 4 M HCl in dioxanes (5 mL). The vial was tightly capped and stirred for 2 h. The reaction remained heterogeneous. An aliquot was taken, diluted with MeOH/DCM (1:1) and analyzed with LCMS to confirm reaction completion. The reaction was concentrated under reduced pressure and dried overnight to give 2 (197 mg) as a white solid HCl salt in quantitative yield. Intermediate 2 (197 mg, 0.459 mmol) was suspended in sieve dried DCM (15 mL) and triethylamine (0.32 mL, 2.3 mmol) and acid-PEG2-tert-butyl ester (140 mg, 0.504 mmol) were added. The reaction was cooled in an ice bath for 10 min and then COMU (216 mg, 0.504 mmol) was added and the reaction was stirred on ice for 10 min. The ice bath was then removed and the reaction was stirred for 3 h warming to room temperature. Reaction completion was confirmed by LCMS. The reaction was diluted with DCM and washed with H₂O (×4), saturated NaHCO₃ (×3), brine, dried over MgSO₄, filtered, and concentrated under reduced pressure to give crude material that was dissolved in DCM (5 mL, sonication required), loaded onto a 24 g silica column, and purified with flash chromatography (MeOH/DCM 0-7% over 60 min, ELS detection, product eluted at 3.75% MeOH) to give 3 (195 mg) as a white solid in 67% yield. LC/MS (ESI⁺) calculated m/z 636.51 (M), found 638.18 (M+H⁺).

Intermediate 3 (195 mg, 0.306 mmol) was stirred in 4 M HCl in dioxane (3.5 mL) in a sealed vial for 2 h. LCMS confirmed reaction completion. The reaction was concentrated under reduced pressure and dried overnight to give 4 (178 mg) as a white solid in quantitative yield. The vial containing 4 (178 mg, 0.306 mmol) was placed under N₂, stir bar added, and triethylamine (0.21 mL, 1.53 mmol), and 2,3,5,6-tetrafluorophenol (56 mg, 0.337 mmol) were added. The reaction was cooled in an ice bath for 10 min and then COMU (144 mg, 0.337 mmol) was added and the reaction was stirred on ice for 10 min. The ice bath was then removed and the reaction was stirred for 3 h warming to room temperature. Reaction completion was confirmed by LCMS. The reaction was diluted with DCM and washed with H₂O (×3), dried over MgSO₄, filtered, and concentrated under reduced pressure to give crude material that was dissolved in DCM (3.5 mL), loaded onto a 12 g silica column, and purified with flash chromatography (EtOAc/hexanes, 0-100% over 30 min, ELS detection, product eluted at 100% EtOAc) to give LP-459-p (82 mg) as a white solid in 37% yield. LC/MS (ESI⁺) calculated m/z 728.44 (M), found 730.17 (M+H⁺)

### Synthesis of LP-460-p

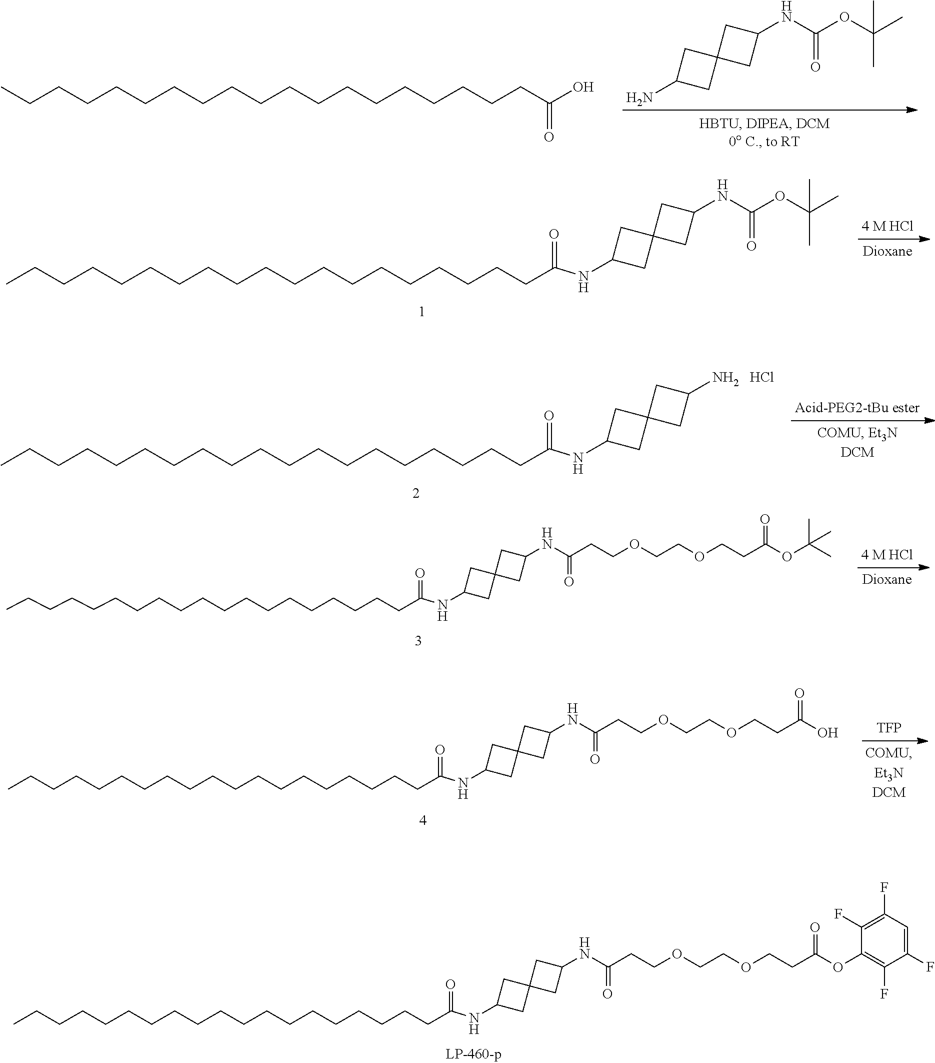

To a 40 mL vial with stir bar and under N₂ was added arachidic acid (300 mg, 0.959 mmol), sieve dried DCM (15 mL), triethylamine (0.53 mL, 3.83 mmol), and N-(2-aminospiro[3.3]hept-6-yl)carbamic acid tert-butyl ester (238 mg, 1.05 mmol). COMU (450 mg, 1.05 mmol) was added to the reaction in one portion and the reaction was stirred for 3 h. TLC (b-cresol green staining) confirmed reaction completion. The reaction was diluted with 10% MeOH/DCM and washed with H₂O (×3) and saturated NaHCO₃ (×4). Large emulsions developed during the washings which needed 20 min to resolve per wash. The pooled organic layer was diluted with MeOH, however never became homogenous. The reaction was not dried with MgSO₄, rather, directly concentrated under reduced pressure, azeotroped with PhMe (10 mL), and then dried on vacuum pump for 14 h. The crude material was suspended in DCM/MeOH (5:1), dry loaded with silica onto a 40 g silica gel column, and purified with flash chromatography (MeOH/DCM 0-15% over 60 min, ELS detection, product eluted around 3% MeOH) to give 1 (250 mg) as a white solid in 50% yield. LC/MS (ESI⁺) calculated m/z 520.46 (M), found 521.90 (M+H⁺).

To the 40 mL vial containing 1 (250 mg, 0.479 mmol) was added stir bar and 4 M HCl in dioxanes (5 mL). The vial was tightly capped and stirred for 2 h. The reaction remained heterogeneous. An aliquot was taken, diluted with MeOH/DCM (1:1) and analyzed with LCMS to confirm reaction completion. The reaction was concentrated under reduced pressure and dried overnight to give 2 (219 mg) as a white solid HCl salt in quantitative yield. Intermediate 2 (219 mg, 0.479 mmol) was suspended in sieve dried DCM (10 mL) and triethylamine (0.33 mL, 2.4 mmol) and acid-PEG2-tert-butyl ester (146 mg, 0.526 mmol) were added. The reaction was cooled in an ice bath for 10 min and then COMU (225 mg, 0.526 mmol) was added and the reaction was stirred on ice for 10 min. The ice bath was then removed and the reaction was stirred for 2 h warming to room temperature. Reaction completion was confirmed by LCMS. The reaction was diluted with DCM and washed with H₂O (×3), saturated NaHCO₃ (×2), brine, dried over MgSO₄, filtered, and concentrated under reduced pressure to give crude material that was dissolved in DCM (10 mL, sonication required), loaded onto a 24 g silica column, and purified with flash chromatography (MeOH/DCM 0-10%, ELS detection) to give intermediate 3 (238 mg) as a white solid in 75% yield. LC/MS (ESI⁺) calculated m/z 664.54 (M), found 666.26 (M+H⁺).

Intermediate 3 (238 mg, 0.358 mmol) was stirred in 4 M HCl in dioxane (7 mL) in a sealed vial for 2.5 h. The reaction started homogenous, but became heterogenous after 30 min. LCMS confirmed reaction completion. The reaction was concentrated under reduced pressure and dried overnight to give 4 (218 mg) as a white solid in quantitative yield. The vial containing 4 (218 mg, 0.358 mmol) was placed under N₂, stir bar added, and triethylamine (0.25 mL, 1.79 mmol), and 2,3,5,6-tetrafluorophenol (65 mg, 0.394 mmol) were added. The reaction was cooled in an ice bath for 10 min and then COMU (169 mg, 0.394 mmol) was added and the reaction was stirred on ice for 10 min. The ice bath was then removed and the reaction was stirred for 2 h warming to room temperature. Reaction completion was confirmed by LCMS. The reaction was diluted with DCM and washed with H₂O (×3), dried over MgSO₄, filtered, and concentrated under reduced pressure to give crude material that was dissolved in DCM (4 mL), loaded onto a 12 g silica column, and purified with flash chromatography (MeOH/DCM 0-3% over 35 min, ELS detection, product eluted at 3% MeOH) to give LP-460-p (81 mg) as a white solid in 30% yield. LC/MS (ESI⁺) calculated m/z 756.47 (M), found 758.16 (M+H⁺).

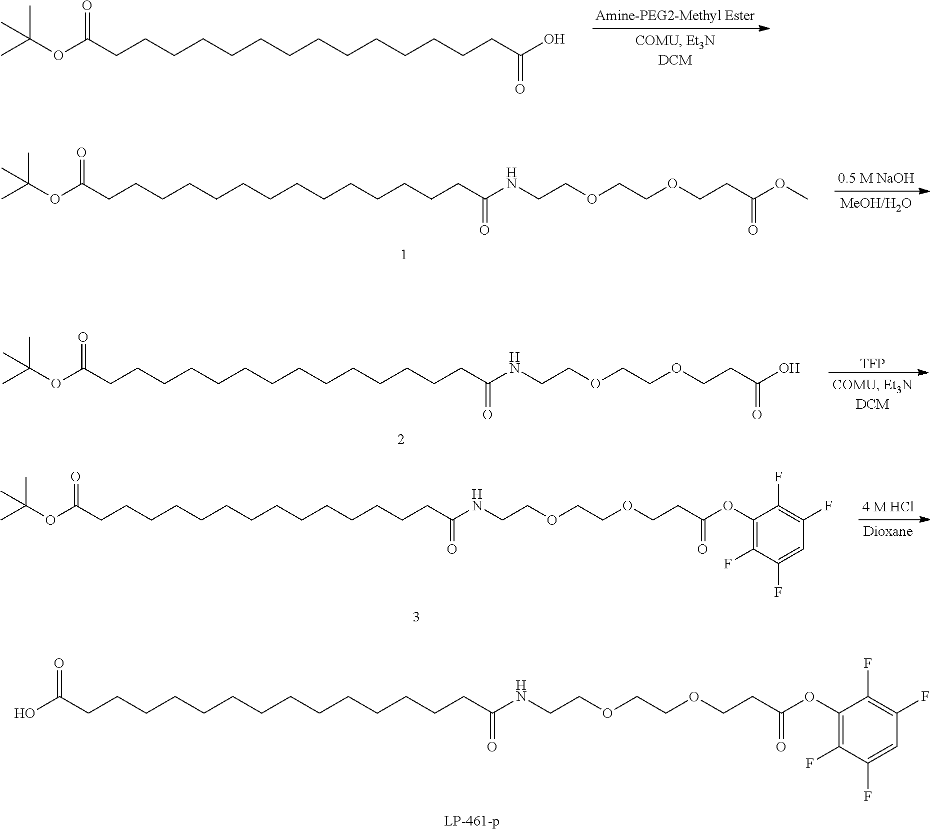

To a 40 mL vial with stir bar and under N₂ was added 16-(tert-butoxy)-16-oxohexadecanoic acid (300 mg, 0.875 mmol), amine-PEG3-methylester HCl salt (219 mg, 0.963 mmol), sieve dried DCM (10 mL), and triethylamine (0.61 mL, 4.38 mmol). The stirred solution was cooled in an ice bath for 10 min and then COMU (412 mg, 0.963 mmol) was added and the reaction was stirred on ice for 10 min. The ice bath was then removed and the reaction was stirred for 2 h warming to room temperature. Reaction completion was confirmed by LCMS and TLC (b-cresol green staining). The reaction was diluted with DCM and washed with H₂O (×3), saturated NaHCO₃ (×2), dried over MgSO₄, filtered, and concentrated under reduced pressure to give crude material that was dissolved in DCM (5 mL), loaded onto a 24 g silica column, and purified with flash chromatography (EtOAc/hexanes 0-100% over 50 min, ELS detection, product eluted at 60% EtOAc) to give 1 (309 mg) as an oily colorless solid in 69% yield. LC/MS (ESI⁺) calculated m/z 515.38 (M), found 516.86 (M+H⁺).

To a 40 mL with stir bar was added 1 (100 mg, 0.194 mmol), MeOH (0.97 mL), and 0.5 M NaOH (0.97 mL, 0.485 mmol). The vial was tightly capped and stirred for 2.5 h. The reaction started heterogenous and slowly became homogeneous. Reaction completion was confirmed by LCMS. The reaction was diluted with H₂O (7 mL), acidified with 1 M HCl until pH was 3, and extracted with DCM (15 mL×3). The pooled organic extracts were washed with brine, dried over MgSO₄, filtered, and concentrated under reduced pressure in a 40 mL vial to give 2 (75 mg) as an oily solid that was placed under N₂ and dissolved in sieve dried DCM (5 mL). To this solution was added stir bar, triethylamine (80 μL, 0.596 mmol), and 2,3,5,6-tetrafluorophenol (30 mg, 0.179 mmol). COMU (77 mg, 0.179 mmol) was added and the reaction was stirred for 3 h. Reaction completion was confirmed with LCMS. The reaction was concentrated under reduced pressure, dried on vacuum pump for 3 h, dissolved in DCM (3 mL), and loaded onto a 12 g silica gel column and purified with flash chromatography (EtOAc/hexanes, 0-100% over 40 min, ELS detection, product eluted at 45% EtOAc) to give 3 (40 mg) a colorless oil in 41% yield. To an oven dried 40 mL vial with stir bar and under N₂ was added 3 (35 mg, 0.0538 mmol), sieve dried dioxane (0.5 mL), and 4 M HCl in dioxane (1.35 mL, 5.38 mmol). The reaction was tightly capped and stirred for 5 h. Reaction completion confirmed by LCMS. The reaction was concentrated at 28° C. and dried on vacuum pump for 16 h to give LP-461-p (30 mg). LC/MS (ESI⁺) calculated m/z 593.30 (M), found 594.98 (M+H⁺).

### Synthesis of LP-468-p

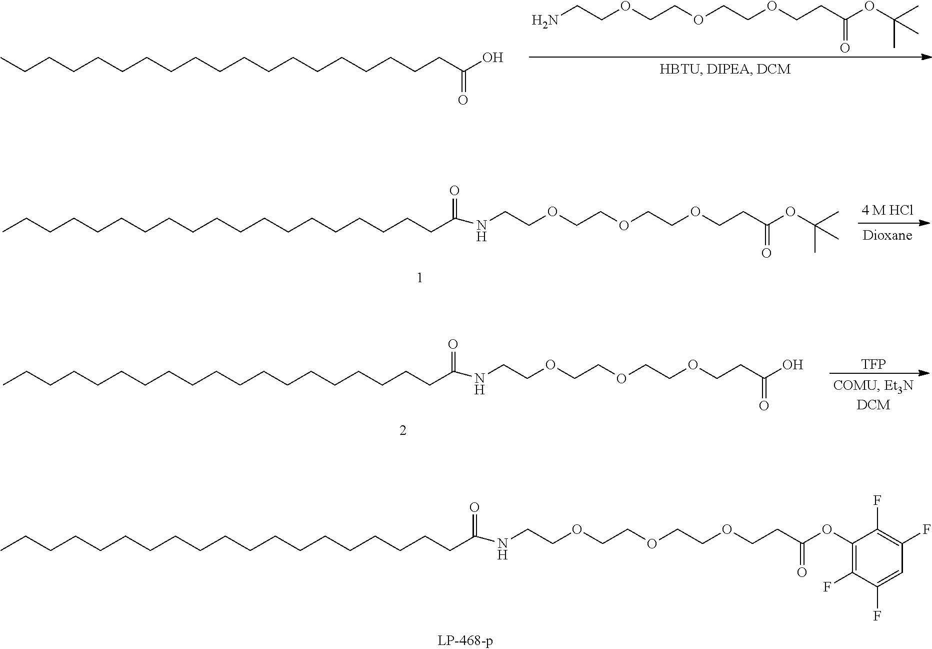

To a 40 mL vial with stir bar and under N₂ was added arachidic acid (300 mg, 0.959 mmol), sieve dried DCM (15 mL), and DIPEA (0.66 mL, 3.83 mmol). HBTU (399 mg, 1.05 mmol) was added and the reaction was stirred for 10 min. Amine-PEG3-tert-butyl ester (292 mg, 1.05 mmol) was added as a solution in DCM (5 mL) and the reaction was stirred for 3.5 h. Reaction completion was confirmed by TLC (b-cresol green staining). The reaction was diluted with DCM, washed with saturated NaHCO₃ (×4), dried over MgSO₄, filtered, and concentrated under reduced pressure to give crude product that was dissolved in DCM (6 mL), loaded onto a 24 g silica gel column, and purified with flash chromatography (EtOAc/hexanes 0-75% over 50 min; ELS detection, product eluted at 50% EtOAc) to give 1 (452 mg) as a white oily foam in 83% yield. The 40 mL vial already containing 1 (450 mg, 0.786 mmol) was stirred in 4 M HCl in dioxane (6 mL) for 2.5 h (tightly capped vial). Reaction completion was confirmed with LCMS. The reaction was concentrated under reduced pressure and dried on vacuum pump for 16 h to give 2 (405 mg). Intermediate 2 (405 mg, 0.78 mmol) was dissolved in sieve dried DCM (15 mL), stir bar added, and placed under N₂. Triethylamine (0.43 mL, 3.12 mmol), 2,3,5,6-tetrafluorophenol (156 mg, 0.94 mmol), and COMU (402 mg, 0.94 mmol) were added consecutively and the reaction was stirred for 2.5 h. The reaction was not complete after this duration (LCMS) so additional 2,3,5,6-tetrafluorophenol (0.6 equiv.) was added and the reaction was stirred for an additional 1.5 h. Reaction completion was confirmed with LCMS. The reaction was diluted with DCM, washed with H₂O (×3), dried over MgSO₄, filtered, and concentrated under reduced pressure to give crude material that was dissolved in DCM (6 mL), loaded onto a 24 g silica gel column, and purified with flash chromatography (EtOAc/hexanes, 0-100% over 45 min, ELS detection). The cleanest fractions were pooled and concentrated under reduced pressure to give LP-468-p (187 mg) as a colorless oil in 36% yield. LC/MS (ESI⁺) calculated m/z 663.41 (M), found 665.09 (M+H⁺).

### Synthesis of LP-469 Phosphoramidite

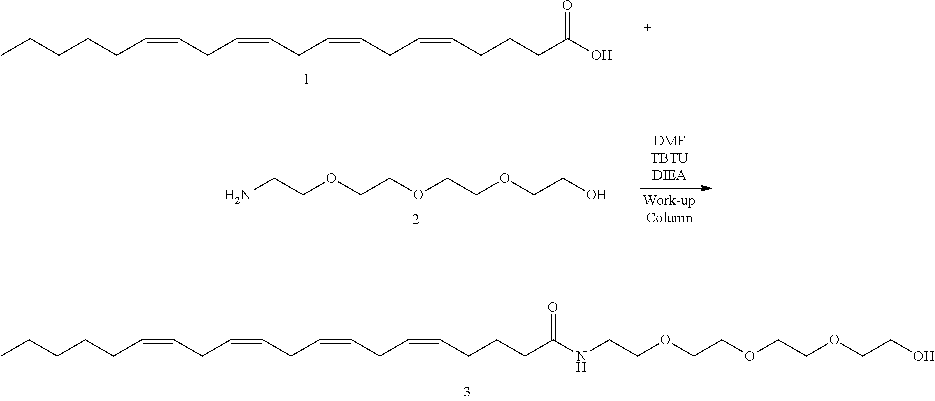 [SMILES: CCCCC/C=C\C/C=C\C/C=C\C/C=C\CCCC(=O)O ; NCCOCCOCCOCCO ; CCCCC/C=C\C/C=C\C/C=C\C/C=C\CCCC(=O)NCCOCCOCCOCCO]

Compound 1 (Asta Tech® #W15452, 500 mg) was dissolved in 11 mL DMF. Then TBTU (606 mg) and DIPEA (1.162 mL) were added and the mixture was stirred in the dark for 10 minutes. Then compound 2 (365 mg in DMF) was added and the mixture was stirred for 1 hour. The mixture was diluted with EtOAc (140 mL) and washed with 3% citric acid in water (3×15 mL), H₂O (2×15 mL), and NaCl (1×15 mL), then dried over Na₂SO₄, filtered and concentrated with rotary evaporator and placed on high vacuum. The crude product was dissolved in 4 mL DCM and loaded onto a 24G column and purified with flash chromatography (MeOH/DCM, 04% over 30 min). Yield 615 mg. LC-MS: calculated [M+H] 479.70, found 480.85.

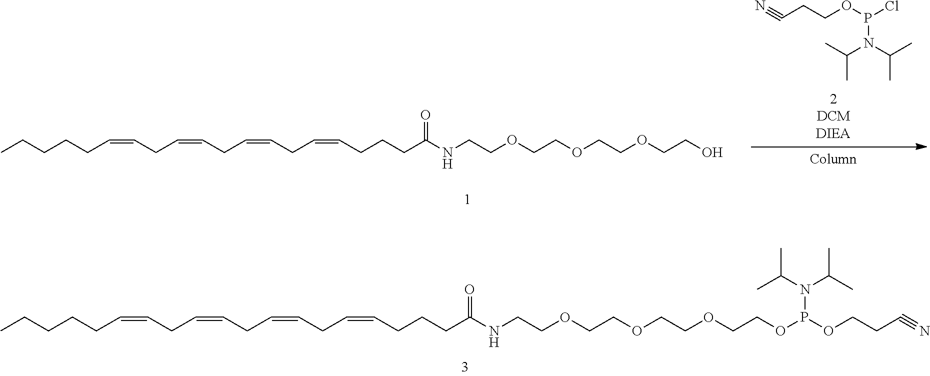 [SMILES: CC(C)N(C(C)C)P(Cl)OCCC#N ; CCCCC/C=C\C/C=C\C/C=C\C/C=C\CCCC(=O)NCCOCCOCCOCCO ; CCCCC/C=C\C/C=C\C/C=C\C/C=C\CCCC(=O)NCCOCCOCCOCCOP(OCCC#N)N(C(C)C)C(C)C]

Compound 1 (613 mg) was added to a 100 mL round bottom flask with sieves and purged with N₂. Then 12 mL of DCM was added, followed by DIPEA (0.678 mL). The mixture was cooled to 0° C. Compound 2 (0.356 mL) was added via syringe dropwise and the mixture was stirred on ice for 10 min., then the ice bath was removed and the reaction mixture was allowed to warm to room temperature and stirred overnight. The reaction was filtered over Celite® and concentrated. The crude product was dry loaded with celite onto a 24G column and purified with flash chromatography (EtOAc/hexanes w/ 1% triethylamine, 0-75% over 35 min). Yield 311 mg. 1H NMR (400 MHz, CD₂Cl₂) δ 6.09 (t, 1H), 5.45-5.32 (m, 8H), 3.85-3.8 (m, 3H), 3.70 (m, 1H), 3.60 (d, 12H), 3.52 (t, 2H), 3.40 (t, 2H), 2.90-2.80 (dd, 6H), 2.65 (t, 2H), 2.18 (t, 2H), 2.16-2.04 (m, 4H), 1.70 (m, 2H), 1.40-1.25 (m, 6H), 1.20 (dd, 12H), 0.80 (t, 3H). 31P NMR (400 MHz, CD₂Cl₂) δ 148.458 (s, 1P).

### Synthesis of LP-470 Phosphoramidite

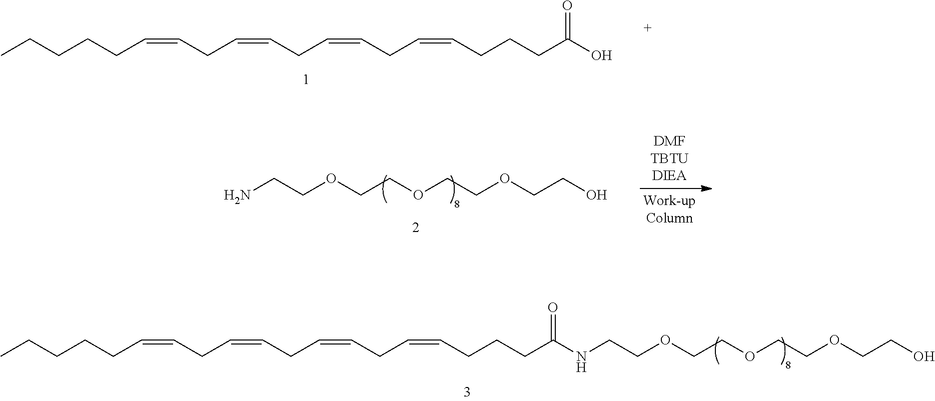

Compound 1 (Asta Tech® #W15452, 250 mg) was dissolved in 10 mL DMF. Then TBTU (303 mg) and DIPEA (0.581 mL) were added. The mixture was stirred for 10 minutes in the dark. Then compound 2 (473 mg in DMF) was added and the reaction was stirred in the dark for 1 hour. The reaction was concentrated with rotary evaporator and placed under high vacuum. The crude material was dissolved in 4 mL DCM and loaded onto a 24G column and purified with flash chromatography (MeOH/DCM, 0-5% over 30 min). Yield 462 mg. LC-MS: calculated [M+H] 788.07, found 789.30.

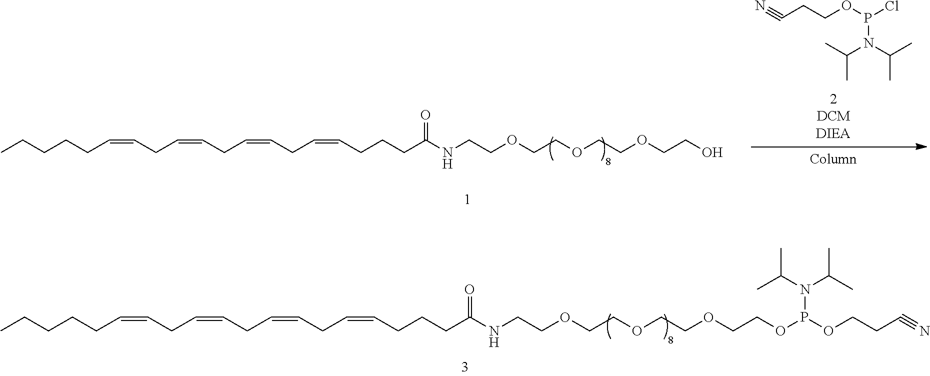

Compound 1 (462 mg) was added to a 100 mL round bottom flask with sieves and purged with N₂. Then 7 mL of DCM was added, followed by DIPEA (0.311 mL) and the mixture was cooled to 0° C. Then compound 2 (0.163 mL) was added via syringe dropwise. The reaction mixture was stirred on ice for 10 min., then the ice bath was removed and the reaction mixture was allowed to warm to room temperature and stirred overnight. The crude reaction was filtered over Celite® and concentrated. The crude product was dry loaded with celite onto a 12G column and purified with flash chromatography (EtOAc/hexanes, 0-100% over 30 min, held at 100% EtOAc for 15 min). Yield: 166 mg 1H NMR (400 MHz, CD₂Cl₂) δ 6.09 (t, 1H), 5.45-5.32 (m, 8H), 3.85-3.8 (m, 3H), 3.70 (m, 1H), 3.60 (d, 40H), 3.52 (t, 2H), 3.40 (t, 2H), 2.90-2.80 (dd, 6H), 2.65 (t, 2H), 2.18 (t, 2H), 2.16-2.04 (m, 4H), 1.70 (m, 2H), 1.40-1.25 (m, 6H), 1.20 (dd, 12H), 0.80 (t, 3H). 31P NMR (400 MHz, CD₂Cl₂) δ 148.458 (s, 1P).

### Synthesis of LP-473-p

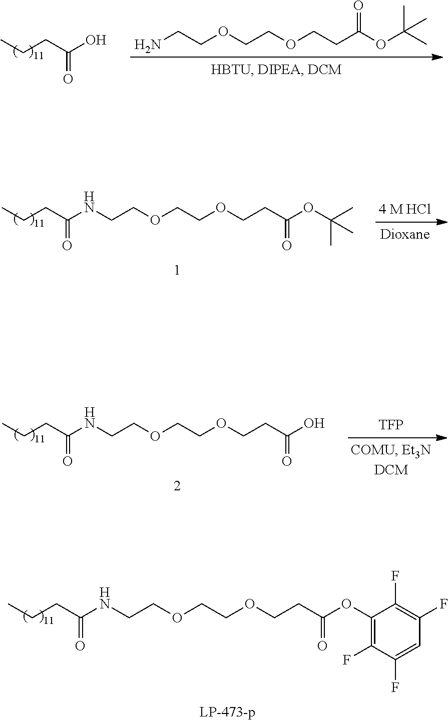

To a 40 mL vial with stir bar and under N₂ was added myristic acid (500 mg, 2.19 mmol), sieve dried DCM (15 mL), and DIPEA (1.5 mL, 8.76 mmol). HBTU (915 mg, 2.41 mmol) was added and the reaction was stirred for 10 min. Amine-PEG2-tert-butyl ester (560 mg, 2.41 mmol) was added as a solution in DCM (5 mL) and the reaction was stirred for 2.5 h. Reaction completion was confirmed by TLC (b-cresol green staining). The reaction was diluted with DCM and washed with H₂O (×2), saturated NaHCO₃ (×3), brine, dried over MgSO₄, filtered, and concentrated under reduced pressure to give crude product that was dissolved in DCM (10 mL), loaded onto a 40 g silica gel column, and purified with flash chromatography (MeOH/DCM 0-5%; ELS detection) to give 1 (1.17 g, oil foam) as a mixture of 1 and tetramethylurea by-product. To an oven dried 100 mL round bottom flask was added 1 (1.17 g), stir bar, and 4 M HCl in dioxane (16.4 mL). The reaction was sealed with a septum and stirred for 3 h. Reaction completion was confirmed with LCMS. The reaction was concentrated under reduced pressure and dried on vacuum pump for 16 h to give crude 2 (846 mg, theoretical max.). In a 100 mL round bottom flask, 2 (846 mg, 2.19 mmol, theoretical max.) was dissolved in sieve dried DCM (20 mL), stir bar added, and the flask was placed under N₂. Triethylamine (1.2 mL, 8.76 mmol) and 2,3,5,6-tetrafluorophenol (437 mg, 2.62 mmol) were added and the reaction was cooled in an ice bath for 10 min. COMU (1.12 g, 2.62 mmol) was added in portions and the reaction was stirred on ice for 30 min. The ice bath was removed and the reaction was stirred for 2 h warming to room temperature. Reaction completion was confirmed with LCMS. The reaction was diluted with DCM, washed with H₂O (×3, 20 mL), dried over MgSO₄, filtered, and concentrated under reduced pressure to give crude material that was dissolved in DCM (10 mL), loaded onto a 24 g silica gel column, and purified with flash chromatography (EtOAc/hexanes, 0-100% over 40 min, ELS detection) to give LP-473-p (558 mg) as a yellow solid. LC/MS (ESI⁺) calculated m/z 535.29 (M), found 536.93 (M+H⁺)

### Synthesis of LP-474-p

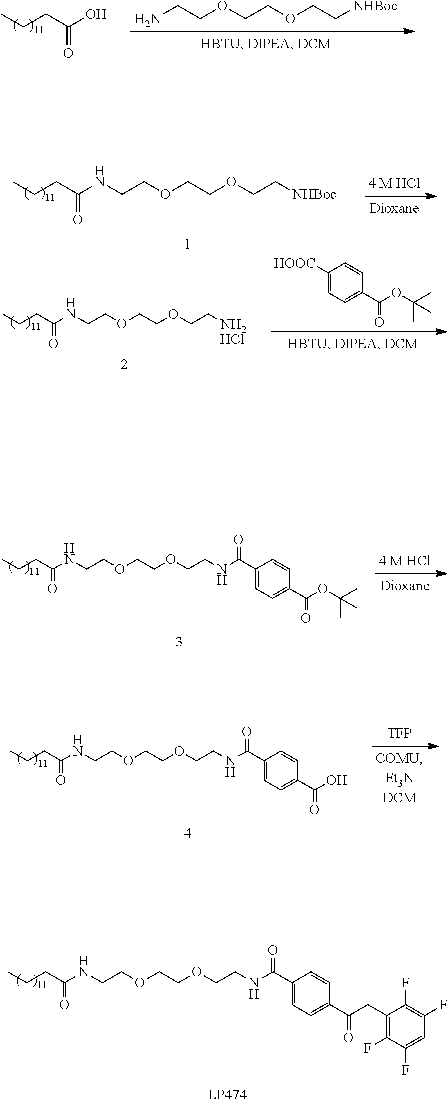

To a 40 mL vial with stir bar and under N₂ was added myristic acid (562 mg, 2.46 mmol), sieve dried DCM (15 mL), and DIPEA (1.7 mL, 9.84 mmol). HBTU (1.12 g, 2.95 mmol) was added and the reaction was stirred for 5 min. Amine-PEG2-NHBoc (675 mg, 2.95 mmol) was added as a solution in DCM (5 mL) and the reaction was stirred for 3 h. Reaction completion was confirmed by TLC (b-cresol green staining). The reaction was diluted with DCM and washed with H₂O (×2), saturated NaHCO₃ (×3), brine, dried over MgSO₄, filtered, and concentrated under reduced pressure to give crude product that was dissolved in DCM (6 mL), loaded onto a 24 g silica gel column, and purified with flash chromatography (EtOAc/hexanes 0-85% over 45 min, ELS detection) to give 1 (916 mg) as a white solid in 82% yield. LC/MS (ESI⁺) calculated m/z 458.37 (M), found 459.79 (M+H⁺).

To the round bottom flask already containing 1 (916 mg, 1.99 mmol) was added stir bar and 4 M HCl in dioxane (12.5 mL). The reaction was sealed with a septum and stirred for 2.5 h. Reaction completion was confirmed with LCMS. The reaction was concentrated under reduced pressure and dried on vacuum pump for 16 h to give crude 2 (789 mg, theoretical max.) that was dissolved in sieve dried DCM (20 mL), stir bar added, and placed under N₂. DIPEA (1 mL, 5.97 mmol) was added and the reaction was stirred for 10 min. 4-(tert-butoxycarbonyl)benzoic acid (529 mg, 2.38 mmol) and HBTU (904 mg, 2.38 mmol) were added consecutively and the reaction was stirred for 4 h. Reaction completion was confirmed by LCMS. The reaction was diluted with DCM and washed with saturated NaHCO₃ (×4), brine, dried over MgSO₄, filtered, and concentrated under reduced pressure to give crude product that was dissolved in DCM (7 mL), loaded onto a 24 g silica gel column, and purified with flash chromatography (EtOAc/hexanes 0-100% over 45 min, ELS detection, product eluted at 70% EtOAc) to give 3 (1 g) as a white solid in 89% yield. LC/MS (ESI⁺) calculated m/z 562.40 (M), found 564.02 (M+H⁺).

To the round bottom flask already containing 3 (1 g, 1.77 mmol) was added stir bar and 4 M HCl in dioxane (17 mL). The reaction was sealed with a septum and stirred for 4 h. Reaction completion was confirmed with LCMS. The reaction was concentrated under reduced pressure and dried on vacuum pump for 4 h to give crude 4 (900 mg, theoretical max.) that was dissolved in sieve dried DCM (20 mL), stir bar added, and placed under N₂. Triethylamine (0.98 mL, 7.08 mmol) and 2,3,5,6-tetrafluorophenol (440 mg, 2.65 mmol) were added and the reaction was cooled in an ice bath for 10 min. COMU (1.13 g, 2.65 mmol) was added in portions and the reaction was stirred on ice for 10 min. The ice bath was removed and the reaction was stirred for 2.5 h warming to room temperature. Reaction completion was confirmed with LCMS. The reaction was concentrated under reduced pressure and dried on vacuum pump for 2 h. The crude material was dissolved in DCM (8 mL), loaded onto a 40 g silica gel column, and purified with flash chromatography (EtOAc/hexanes, 0-100% over 50 min, ELS detection) to give LP-474-p (1.3 g, mixture of product and urea by-product) as an orange solid that was not further purified. LC/MS (ESI⁺) calculated m/z 654.33 (M), found 656.00 (M+H⁺).

### Synthesis of CNR1 SM2-p

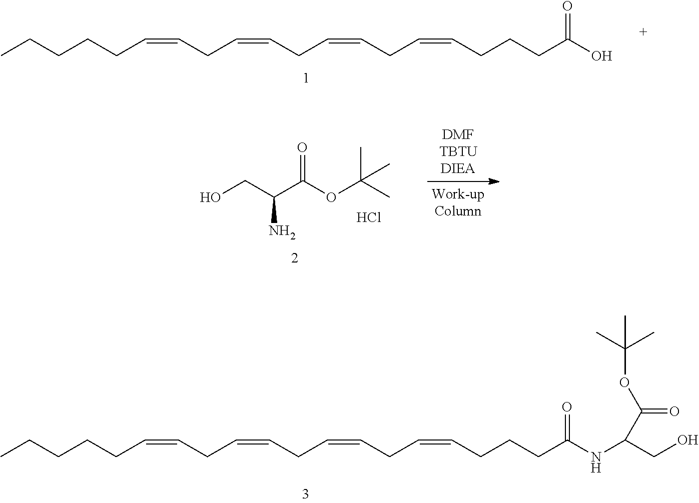 [SMILES: CCCCC/C=C\C/C=C\C/C=C\C/C=C\CCCC(=O)O ; CC(C)(C)OC(=O)[C@@H](N)CO ; Cl ; CCCCC/C=C\C/C=C\C/C=C\C/C=C\CCCC(=O)NC(CO)C(=O)OC(C)(C)C]

Compound 1 (Asta Tech® #W15452, 915 mg) was dissolved in 20 mL DMF. Then TBTU (1.1 g) and DIPEA (2.1 mL) were added and the mixture was stirred for 10 minutes. Then compound 2 (Asta Tech #F11105, 677 mg) was added and the reaction was covered in foil and allowed to stir for 1 hour. Then the reaction was diluted with 200 mL EtOAc and washed with 3% citric acid in water (3×25 mL), H₂O (2×25 mL), NaCl (1×25 mL), then dried over Na₂SO₄, filtered and concentrated with rotary evaporator. The crude product was dissolved in 5 mL DCM and loaded onto a 40 G column and purified with flash chromatography (MeOH/DCM, 0-3% over 30 min). Yield 977 mg. LC-MS: calculated [M+H] 447.66, found 448.87.

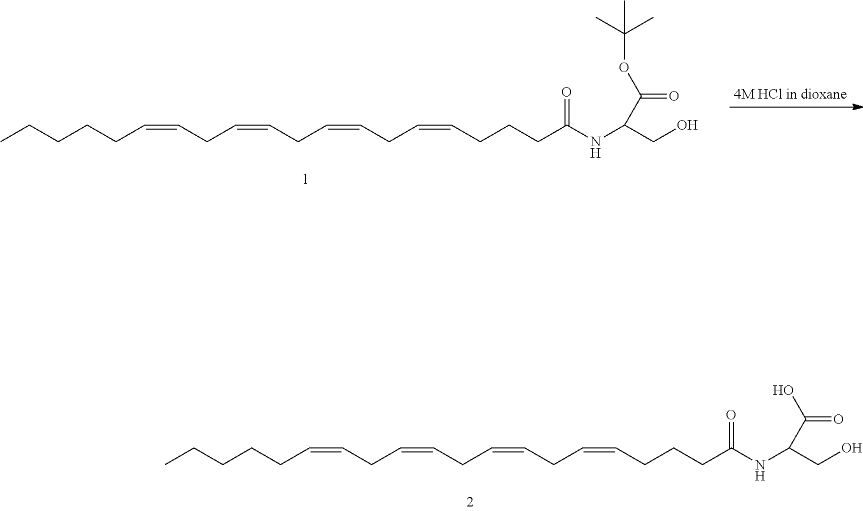 [SMILES: CCCCC/C=C\C/C=C\C/C=C\C/C=C\CCCC(=O)NC(CO)C(=O)OC(C)(C)C ; CCCCC/C=C\C/C=C\C/C=C\C/C=C\CCCC(=O)NC(CO)C(=O)O]

Compound 1 (977 mg) was dissolved in 12 mL 4M HCl in dioxane at 0° C. and stirred for 10 minutes. Then the flask was covered in foil and the reaction mixture was allowed to warm to room temperature and stirred for 5 hours. The product was concentrated on rotary evaporator and placed under high vacuum, then dissolved twice in DCM/toluene, concentrated, and placed under high vacuum overnight. Yield 879 mg. LC-MS: calculated [M+H] 391.55, found 392.83.

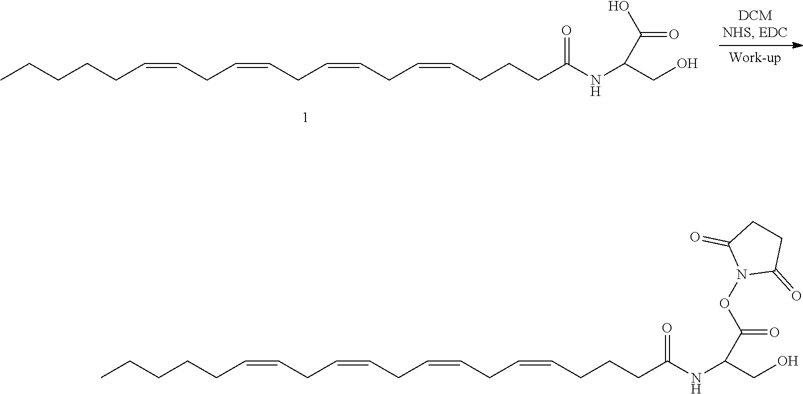 [SMILES: CCCCC/C=C\C/C=C\C/C=C\C/C=C\CCCC(=O)NC(CO)C(=O)O ; CCCCC/C=C\C/C=C\C/C=C\C/C=C\CCCC(=O)NC(CO)C(=O)ON1C(=O)CCC1=O]

Compound 1 (854 mg) was dissolved in 20 mL DCM. Then NHS (Sigma® #130672, 251 mg) was added and the mixture was allowed to stir for 10 minutes. Then EDC HCl (Sigma® #E7750, 418 mg) was added and the reaction mixture was stirred for 4 hours in the dark. The reaction mixture was diluted with 90 mL DCM, washed with 3% citric acid in water (2×10 mL), H₂O (1×10 mL), then NaCl (1×10 mL), then dried over Na₂SO₄, filtered and concentrated. Yield 970 mg. Product used without further purification. LC-MS: calculated [M+H] 488.63, found 489.79.

### Example 3. Conjugation of Lipid PK/PD Modulator Precursors

Either prior to or after annealing and prior to or after conjugation of one or more targeting ligands, one or more lipid PK/PD modulator precursors can be linked to an oligonucleotide-based agent. The following describes the general conjugation process used to link lipid PK/PD modulator precursors to the RNAi agent constructs set forth in the Examples depicted herein.

### A. Conjugation of Activated Ester PK/PD Modulators

The following procedure was used to conjugate PK/PD modulators having an activated ester moiety such as TFP (tetrafluorophenoxy) or PNP (para-nitrophenol) to an RNAi agent with an amine-functionalized sense strand, such as C6-NH2, NH2-C6, or (NH2-C6). An annealed RNAi Agent dried by lyophilization was dissolved in DMSO and 10% water (v/v %) at 25 mg/mL. Then 50-100 equivalents of TEA and 3 equivalents of activated ester PK/PD modulator were added to the solution. The solution was allowed to react for 1-2 hours, while monitored by RP-HPLC-MS (mobile phase A 100 mM HFIP, 14 mM TEA; mobile phase B: acetonitrile on an Waters™ XBridge C18 column, Waters Corp.)

The product was then precipitated by adding 12 mL acetonitrile and 0.4 mL PBS and centrifuging the solid to a pellet. The pellet was then re-dissolved in 0.4 mL of 1×PBS and 12 mL of acetonitrile. The resulting pellet was dried on high vacuum for one hour.

### B. Conjugation of a Maleimide-Containing Lipid PK/PD Modulator Precursor

The following describes the general process used to link a maleimide-containing lipid PK/PD modulator precursor to the (C6-SS-C6) or (6-SS-6) functionalized sense strand of an RNAi agent by undertaking a dithiothreitol reduction of disulfide followed by a thiol-Michael Addition of the respective maleimide-containing lipid PK/PD modulator precursor: In a vial, functionalized sense strand was dissolved at 50 mg/mL in sterilized water. Then 20 equivalents of each of 0.1M Hepes pH 8.5 buffer and dithiothreitol were added. The mixture was allowed to react for one hour, then the conjugate was precipitated in acetonitrile and PBS, and the solids were centrifuged into a pellet.

The pellet was brought up in a 70/30 mixture of DMSO/water at a solids concentration of 30 mg/mL. Then, the maleimide-containing lipid PK/PD modulator precursor was added at 1.5 equivalents. The mixture was allowed to react for 30 minutes. The product was purified on an AEX-HPLC (mobile phase A: 25 mM TRIS pH=7.2, 1 mM EDTA, 50% acetonitrile; mobile phase B: 25 mM TRIS pH=7.2, 1 mM EDTA, 500 mM NaBr, 50% acetonitrile; solid phase TSKgel-30; 1.5 cm×10 cm.) The solvent was removed by rotary evaporator, and desalted with a 3K spin column using 2×10 mL exchanges with sterilized water. The solid product was dried using lyophilization and stored for later use.

### C. Conjugation of a Sulfone-Containing Lipid PK/PD Modulator Precursor

In a vial, functionalized sense strand was dissolved at 50 mg/mL in sterilized water. Then 20 equivalents of each of 0.1M Hepes pH 8.5 buffer and dithiothreitol are added. The mixture was allowed to react for one hour, then the conjugate was precipitated in acetonitrile and PBS, and the solids were centrifuged into a pellet.

The pellet was brought up in a 70/30 mixture of DMSO/water at a solids concentration of 30 mg/mL. Then, the sulfone-containing lipid PK/PD modulator precursor was added at 1.5 equivalents. The vial was purged with N₂, and heated to 40° C. while stirring. The mixture was allowed to react for one hour. The product was purified on an AEX-HPLC (mobile phase A: 25 mM TRIS pH=7.2, 1 mM EDTA, 50% acetonitrile; mobile phase B: 25 mM TRIS pH=7.2, 1 mM EDTA, 500 mM NaBr, 50% acetonitrile; solid phase TSKgel-30; 1.5 cm×10 cm.) The solvent was removed by rotary evaporator, and desalted with a 3K spin column using 2×10 mL exchanges with sterilized water. The solid product was dried using lyophilization and stored for later use.

### D. Conjugation of an Azide-Containing Lipid PK/PD Modulator Precursor

One molar equivalent of TG-TBTA resin loaded with Cu(I) was weighed into a glass vial. The vial was purged with N₂ for 15 minutes. Then, functionalized sense strand was dissolved in a separate vial in sterilized water at a concentration of 100 mg/mL. Then two equivalents of the azide-containing lipid PK/PD modulator precursor (50 mg/mL in DMF) is added to the vial. Then TEA, DMF and water are added until the final reaction conditions are 33 mM TEA, 60% DMF, and 20 mg/mL of the conjugated product. The solution was then transferred to the vial with resin via a syringe. The N₂ purge was removed and the vial was sealed and moved to a stir plate at 40° C. The mixture was allowed to react for 16 hours. The resin was filtered off using a 0.45 μm filter.

The product was purified using AEX purification (mobile phase A: 25 mM TRIS pH=7.2, 1 mM EDTA, 50% acetonitrile; mobile phase B: 25 mM TRIS pH=7.2, 1 mM EDTA, 500 mM NaBr, 50% acetonitrile solid phase TSKgel-30; 1.5 cm×10 cm.) The acetonitrile was removed using a rotary evaporator, and desalted with a 3K spin column using 2×10 mL exchanges with sterilized water. The solid product was dried using lyophilization and stored for later use.

TABLE A
Conjugate ID Numbers With Chemically Modified Antisense and Sense Strands
(including Linkers and Table A Conjugates) used in the following Examples.

| | | SEQ | | SEQ |
|---|---|---|---|---|
| AC/AD ID | Sense Strand (Fully Modified with Conjugated | ID | | ID |
| Number | Targeting Ligand) (5′→3′) | NO: | Antisense Strand (5′→3′) | NO: |
| AC002571 | LP-249-a-(NH-C6) | 10 | cPrpasGfsusaaauguagAfgUfcGfuugasc | 1 |
| | s(invAb)sgucaacgaCfUfCfuacauuuacus(invAb)(C6-S)-NEM | | | |
| AC002574 | LP249-(NH- | 11 | cPrpasGfsusaaauguagAfgUfcGfuugasc | 1 |
| | C6)s(invAb)sgucaacgaCfUfCfuacauuuacus(invAb)(C6-S)-LP- | | | |
| | 359-a | | | |
| AC002577 | LP-379-a-L6-(NH- | 12 | cPrpasGfsusaaauguagAfgUfcGfuugasc | 1 |
| | C6)s(invAb)sgucaacgaCfUfCfuacauuuacus(invAb)(C6-S)-LP- | | | |
| | 378-a | | | |
| AC002626 | WAT Homing Pep-DBCO- | 13 | cPrpasGfsusaaauguagAfgUfcGfuugasc | 1 |
| | C6s(invAb)sgucaacgaCfUfCfuacauuuacus(invAb)(C6-S)-LP- | | | |
| | 371-a | | | |
| AC002648 | LP-183-a-(NH- | 14 | cPrpasGfsusaaauguagAfgUfcGfuugasc | 1 |
| | C6)s(invAb)sgucaacgaCfUfCfuacauuuacus(invAb)(C6-S)-NEM | | | |
| AC002649 | LP-379-a-L6-(NH- | 15 | cPrpasGfsusaaauguagAfgUfcGfuugasc | 1 |
| | C6)s(invAb)sgucaacgaCfUfCfuacauuuacus(invAb)(C6-S)-LP- | | | |
| | 371-a | | | |
| AC002650 | CNR1 SM2-1-a-L6-(NH- | 16 | cPrpasGfsusaaauguagAfgUfcGfuugasc | 1 |
| | C6)s(invAb)sgucaacgaCfUfCfuacauuuacus(invAb)(C6-S)-LP- | | | |
| | 378-a | | | |
| AC002675 | LP-377-a-(NH- | 17 | cPrpasGfsusaaauguagAfgUfcGfuugasc | 1 |
| | C6)s(invAb)sgucaacgaCfUfCfuacauuuacus(invAb)(C6-S)-NEM | | | |
| AC002679 | LP-403-a L6-(NH- | 18 | cPrpasGfsusaaauguagAfgUfcGfuugasc | 1 |
| | C6)s(invAb)sgucaacgaCfUfCfuacauuuacus(invAb)(C6-S)-LP- | | | |
| | 378-a | | | |
| AC002680 | Ac-(NH-C6)s(invAb)sgucaacgaCfUfCfuacauuuacus(invAb)(C6- | 19 | cPrpasGfsusaaauguagAfgUfcGfuugasc | 1 |
| | S)-LP-404-a | | | |
| AC002681 | LP-403-a-L6-(NH- | 20 | cPrpasGfsusaaauguagAfgUfcGfuugasc | 1 |
| | C6)s(invAb)sgucaacgaCfUfCfuacauuuacus(invAb)(C6-S)-LP- | | | |
| | 404-a | | | |
| AC002703 | LP-379-a-L6-(NH- | 21 | cPrpasGfsusaaauguagAfgUfcGfuugasc | 1 |
| | C6)s(invAb)sgucaacgaCfUfCfuacauuuacus(invAb)(C6-S)-LP- | | | |
| | 361-a | | | |
| AC002704 | LP-211-a-L6-(NH- | 22 | cPrpasGfsusaaauguagAfgUfcGfuugasc | 1 |
| | C6)s(invAb)sgucaacgaCfUfCfuacauuuacus(invAb)(C6-S)-NEM | | | |
| AC002705 | LP-379-a-L6-(NH- | 23 | cPrpasGfsusaaauguagAfgUfcGfuugasc | 1 |
| | C6)s(invAb)sgucaacgaCfUfCfuacauuuacus(invAb)(C6-S)-LP- | | | |
| | 359-a | | | |
| AC002706 | Ac-(NH-C6)s(invAb)sgucaacgaCfUfCfuacauuuacus(invAb)(C6- | 24 | cPrpasGfsusaaauguagAfgUfcGfuugasc | 1 |
| | S)-LP-374-a | | | |
| AC002707 | Ac-(NH-C6)s(invAb)sgucaacgaCfUfCfuacauuuacus(invAb)(C6- | 25 | cPrpasGfsusaaauguagAfgUfcGfuugasc | 1 |
| | S)-LP-375-a | | | |
| AC002709 | TDA Pep-1-DBCO- | 26 | cPrpasGfsusaaauguagAfgUfcGfuugasc | 1 |
| | C6s(invAb)sgucaacgaCfUfCfuacauuuacus(invAb)(C6-S)-LP- | | | |
| | 371-a | | | |
| AC002739 | LP-232-a-(NH- | 27 | cPrpasGfsusaaauguagAfgUfcGfuugasc | 1 |
| | C6)s(invAb)sgucaacgaCfUfCfuacauuuacus(invAb)(C6-S)-NEM | | | |
| AC002740 | LP-242-a-(NH- | 28 | cPrpasGfsusaaauguagAfgUfcGfuugasc | 1 |
| | C6)s(invAb)sgucaacgaCfUfCfuacauuuacus(invAb)(C6-S)-NEM | | | |
| AC002743 | LP-18-a-L6-(NH- | 29 | cPrpasGfsusaaauguagAfgUfcGfuugasc | 1 |
| | C6)s(invAb)sgucaacgaCfUfCfuacauuuacus(invAb)(C6-S)-NEM | | | |
| AC002744 | LP-18-a-L6-(NH- | 30 | cPrpasGfsusaaauguagAfgUfcGfuugasc | 1 |
| | C6)s(invAb)sgucaacgaCfUfCfuacauuuacus(invAb)(C6-S)-LP- | | | |
| | 378-a | | | |
| AC002745 | LP-274-a-(NH- | 31 | cPrpasGfsusaaauguagAfgUfcGfuugasc | 1 |
| | C6)s(invAb)sgucaacgaCfUfCfuacauuuacus(invAb)(C6-S)-LP- | | | |
| | 378-a | | | |
| AC002746 | LP-295-a-(NH- | 32 | cPrpasGfsusaaauguagAfgUfcGfuugasc | 1 |
| | C6)s(invAb)sgucaacgaCfUfCfuacauuuacus(invAb)(C6-S)-LP- | | | |
| | 378-a | | | |
| AC002772 | Ac-(NH-C6)s(invAb)sgucaacgaCfUfCfuacauuuacus(invAb)(C6- | 33 | cPrpasGfsusaaauguagAfgUfcGfuugasc | 1 |
| | S)-NEM | | | |
| AC002773 | LP-310-a-(NH- | 34 | cPrpasGfsusaaauguagAfgUfcGfuugasc | 1 |
| | C6)s(invAb)sgucaacgaCfUfCfuacauuuacus(invAb)(C6-S)-NEM | | | |
| AC002785 | CNR1-SM2-a-(NH- | 35 | cPrpasGfsusaaauguagAfgUfcGfuugasc | 1 |
| | C6)s(invAb)sgucaacgaCfUfCfuacauuuacus(invAb)(C6-S)-LP- | | | |
| | 378-a | | | |
| AC002786 | CNR1 SM2-a-(NH- | 36 | cPrpasGfsusaaauguagAfgUfcGfuugasc | 1 |
| | C6)s(invAb)sgucaacgaCfUfCfuacauuuacus(invAb)(C6-S)-LP- | | | |
| | 380-a | | | |
| AC002787 | CNR1 SM2-a-(NH-C6)s(invAb) | 37 | cPrpasGfsusaaauguagAfgUfcGfuugasc | 1 |
| | sgucaacgaCfUfCfuacauuuacus(invAb)(C6-S)-LP-4-a | | | |
| AC002788 | CNR1 SM2-a-(NH- | 38 | cPrpasGfsusaaauguagAfgUfcGfuugasc | 1 |
| | C6)s(invAb)sgucaacgaCfUfCfuacauuuacus(invAb)(C6-S)-LP- | | | |
| | 359-a | | | |
| AC002795 | LP-245-a-(NH- | 39 | cPrpasGfsusaaauguagAfgUfcGfuugasc | 1 |
| | C6)s(invAb)sgucaacgaCfUfCfuacauuuacus(invAb)(C6-S)-NEM | | | |
| AC002796 | LP-243-a-(NH- | 40 | cPrpasGfsusaaauguagAfgUfcGfuugasc | 1 |
| | C6)s(invAb)sgucaacgaCfUfCfuacauuuacus(invAb)(C6-S)-NEM | | | |
| AC002797 | LP-379-a-L6-(NH- | 41 | cPrpasGfsusaaauguagAfgUfcGfuugasc | 1 |
| | C6)s(invAb)sgucaacgaCfUfCfuacauuuacus(invAb)(C6-S)-LP- | | | |
| | 151-a | | | |
| AC002873 | LP-413-a-(NH- | 42 | cPrpasGfsusaaauguagAfgUfcGfuugasc | 1 |
| | C6)s(invAb)sgucaacgaCfUfCfuacauuuacus(invAb)(C6-S)-LP- | | | |
| | 378-a | | | |
| AC002874 | LP-412-a-(NH- | 43 | cPrpasGfsusaaauguagAfgUfcGfuugasc | 1 |
| | C6)s(invAb)sgucaacgaCfUfCfuacauuuacus(invAb)(C6-S)-LP- | | | |
| | 378-a | | | |
| AC002911 | LP-424-a-(NH- | 44 | cPrpasGfsusaaauguagAfgUfcGfuugasc | 1 |
| | C6)s(invAb)sgucaacgaCfUfCfuacauuuacus(invAb)(C6-S)-LP- | | | |
| | 378-a | | | |
| AC002912 | LP-426-a-(NH- | 45 | cPrpasGfsusaaauguagAfgUfcGfuugasc | 1 |
| | C6)s(invAb)sgucaacgaCfUfCfuacauuuacus(invAb)(C6-S)-LP- | | | |
| | 378-a | | | |
| AC002913 | LP-425-a-(NH- | 46 | cPrpasGfsusaaauguagAfgUfcGfuugasc | 1 |
| | C6)s(invAb)sgucaacgaCfUfCfuacauuuacus(invAb)(C6-S)-LP- | | | |
| | 378-a | | | |
| AC002914 | LP-427-a-(NH- | 47 | cPrpasGfsusaaauguagAfgUfcGfuugasc | 1 |
| | C6)s(invAb)sgucaacgaCfUfCfuacauuuacus(invAb)(C6-S)-LP- | | | |
| | 378-a | | | |
| AC002915 | LP-200-a-(NH- | 48 | cPrpasGfsusaaauguagAfgUfcGfuugasc | 1 |
| | C6)s(invAb)sgucaacgaCfUfCfuacauuuacus(invAb)(C6-S)-LP- | | | |
| | 378-a | | | |
| AC002954 | LP-413-a-(NH- | 49 | cPrpasGfsusaaauguagAfgUfcGfuugasc | 1 |
| | C6)s(invAb)sgucaacgaCfUfCfuacauuuacus(invAb)LP-428-a | | | |
| AC003018 | LP-432-a-(NH- | 50 | cPrpasGfsusaaauguagAfgUfcGfuugasc | 1 |
| | C6)s(invAb)sgucaacgaCfUfCfuacauuuacus(invAb)(C6-S)-LP- | | | |
| | 378-a | | | |
| AC003019 | LP-433-a-(NH- | 51 | cPrpasGfsusaaauguagAfgUfcGfuugasc | 1 |
| | C6)s(invAb)sgucaacgaCfUfCfuacauuuacus(invAb)(C6-S)-LP- | | | |
| | 378-a | | | |
| AC003020 | LP-447-a-(NH- | 52 | cPrpasGfsusaaauguagAfgUfcGfuugasc | 1 |
| | C6)s(invAb)sgucaacgaCfUfCfuacauuuacus(invAb)(C6-S)-LP- | | | |
| | 378-a | | | |
| AC003021 | LP-128-a-(NH- | 53 | cPrpasGfsusaaauguagAfgUfcGfuugasc | 1 |
| | C6)s(invAb)sgucaacgaCfUfCfuacauuuacus(invAb)(C6-S)-LP- | | | |
| | 378-a | | | |
| AC003022 | LP-445-a-(NH- | 54 | cPrpasGfsusaaauguagAfgUfcGfuugasc | 1 |
| | C6)s(invAb)sgucaacgaCfUfCfuacauuuacus(invAb)(C6-S)-NEM | | | |
| AC003023 | LP-444-a-(NH- | 55 | cPrpasGfsusaaauguagAfgUfcGfuugasc | 1 |
| | C6)s(invAb)sgucaacgaCfUfCfuacauuuacus(invAb)(C6-S)-NEM | | | |
| AC003024 | LP-446-a-(NH- | 56 | cPrpasGfsusaaauguagAfgUfcGfuugasc | 1 |
| | C6)s(invAb)sgucaacgaCfUfCfuacauuuacus(invAb)(C6-S)-NEM | | | |
| AC003025 | LP-453-a-(NH- | 57 | cPrpasGfsusaaauguagAfgUfcGfuugasc | 1 |
| | C6)s(invAb)sgucaacgaCfUfCfuacauuuacus(invAb)(C6-S)-NEM | | | |
| AC003031 | LP-244-a-(NH- | 58 | cPrpasGfsusaaauguagAfgUfcGfuugasc | 1 |
| | C6)s(invAb)sgucaacgaCfUfCfuacauuuacus(invAb)(C6-S)-NEM | | | |
| AC003058 | LP-455-a-(NH- | 59 | cPrpasGfsusaaauguagAfgUfcGfuugasc | 1 |
| | C6)s(invAb)sgucaacgaCfUfCfuacauuuacus(invAb)(C6-S)-LP- | | | |
| | 378-a | | | |
| AC003059 | LP-457-a-(NH- | 60 | cPrpasGfsusaaauguagAfgUfcGfuugasc | 1 |
| | C6)s(invAb)sgucaacgaCfUfCfuacauuuacus(invAb)(C6-S)-LP- | | | |
| | 378-a | | | |
| AC003060 | LP-458-a-(NH- | 61 | cPrpasGfsusaaauguagAfgUfcGfuugasc | 1 |
| | C6)s(invAb)sgucaacgaCfUfCfuacauuuacus(invAb)(C6-S)-LP- | | | |
| | 378-a | | | |
| AC003061 | LP-459-a-(NH- | 62 | cPrpasGfsusaaauguagAfgUfcGfuugasc | 1 |
| | C6)s(invAb)sgucaacgaCfUfCfuacauuuacus(invAb)(C6-S)-NEM | | | |
| AC003062 | LP-460-a-(NH- | 63 | cPrpasGfsusaaauguagAfgUfcGfuugasc | 1 |
| | C6)s(invAb)sgucaacgaCfUfCfuacauuuacus(invAb)(C6-S)-NEM | | | |
| AC003063 | LP-461-a-(NH- | 64 | cPrpasGfsusaaauguagAfgUfcGfuugasc | 1 |
| | C6)s(invAb)sgucaacgaCfUfCfuacauuuacus(invAb)(C6-S)-LP- | | | |
| | 371-a | | | |
| AC003103 | LP-208-a-(NH- | 65 | cPrpasGfsusaaauguagAfgUfcGfuugasc | 1 |
| | C6)s(invAb)sgucaacgaCfUfCfuacauuuacus(invAb)(C6-S)-LP- | | | |
| | 378-a | | | |
| AC003104 | LP-446-a-(NH- | 66 | cPrpasGfsusaaauguagAfgUfcGfuugasc | 1 |
| | C6)s(invAb)sgucaacgaCfUfCfuacauuuacus(invAb)(C6-S)-LP- | | | |
| | 371-a | | | |
| AC003105 | LP-446-a-(NH- | 67 | cPrpasGfsusaaauguagAfgUfcGfuugasc | 1 |
| | C6)s(invAb)sgucaacgaCfUfCfuacauuuacus(invAb)(C6-S)-LP- | | | |
| | 378-a | | | |
| AC003144 | LP-468-a-(NH- | 68 | cPrpasGfsusaaauguagAfgUfcGfuugasc | 1 |
| | C6)s(invAb)sgucaacgaCfUfCfuacauuuacus(invAb)(C6-S)-LP- | | | |
| | 378-a | | | |
| AC003478 | Ac-(NH-C6)s(invAb)sgucaacgaCfUfCfuacauuuacus(invAb)(C6- | 68 | cPrpasGfsusaaauguagAfgUfcGfuugasc | 1 |
| | S)-LP-416-a | | | |
| AC004390 | LP379-a-L6-(NH- | 69 | cPrpusAfsugauAfgaauCfgUfgCfuucasc | 2 |
| | C6)s(invAb)sgugaagcaCfGfAfuucuaucauas(invAb)(C6-S)-LP- | | | |
| | 371-a | | | |
| AC004391 | LP379-a-L6-(NH- | 70 | cPrpusAfsggauUfcuggGfgUfaUfgucusc | 3 |
| | C6)s(invAb)sgagacauaCfCfCfcagaauccuas(invAb)(C6-S)-LP- | | | |
| | 371-a | | | |
| AC004392 | LP379-a-L6-(NH- | 71 | cPrpusCfsgaauAfgaugUfcAfgCfucgcsu | 4 |
| | C6)s(invAb)sagcgagcuGfAfCfaucuauucgas(invAb)(C6-S)-LP- | | | |
| | 371-a | | | |
| AC005181 | LP379-a-L6-(NH- | 72 | cPrpusAfsugauagaauCfgUfgCfuucasc | 5 |
| | C6)s(invAb)sgugaagcaCfGfAfuucuaucauas(invAb)(C6-S)-LP- | | | |
| | 371-a | | | |
| AC005823 | LP379-a-L6-(NH- | 73 | cPrpusGfsuauc AfucaaGfcAfuUfucagsg | 6 |
| | C6)s(invAb)sccugaaauGfCfUfugaugauacas(invAb)(C6-S)-LP- | | | |
| | 371-a | | | |
| AC005824 | LP379-a-L6-(NH- | 74 | cPrpusAfsuuguAfucauCfaAfgCfauuusc | 7 |
| | C6)s(invAb)sga_2NaaugcuUfGfAfugauacaa_2Nuas(invAb)(C6- | | | |
| | S)-LP-371-a | | | |
| AC006188 | LP379-a-L6-(NH- | 75 | cPrpasUfsuguuGfgaacUfaUfgAfcagasc | 8 |
| | C6)s(invAb)sgucugucaUfAfGfuuccaacaaus(invAb)(C6-S)-LP- | | | |
| | 371-a | | | |
| AC006189 | LP379-a-L6-(NH- | 76 | cPrpusCfsaugaUfauucAfgAfuAfccagsc | 9 |
| | C6)s(invAb)sgcugguauCfUfGfaauaucaugas(invAb)(C6-S)-LP- | | | |
| | 371-a | | | |

(Ac = acetyl);
(NH-C6)s, (invAb), (C6-S), L6, NEM, DBCO-C6s, TDA Pep 1, WAT Homing Pep,: See Table 4 for chemical structure information.

### Example 4. In Vivo Administration of Lipid-Conjugated RNAi Agents in Mice

On study day 1, female C57bl/6 mice were injected with either saline or a lipid-conjugated RNAi agent formulated in saline. Five (n=5) animals were dosed in each group at 250 μL/25 g body weight with saline or RNAi agent solution (at 1 mg/kg). Animals were injected subcutaneously (SQ) to the dosing regimen of Table 5.

TABLE 5
Dosing regimen for mice of Example 4.

| Group | Dosing Material | Dosing Regimen |
|---|---|---|
| 1 | Saline | Single SQ Injection on Day 1 |
| 2 | 1 mg/kg AC002785 | Single SQ Injection on Day 1 |
| 3 | 1 mg/kg AC002873 | Single SQ Injection on Day 1 |
| 4 | 1 mg/kg AC002954 | Single SQ Injection on Day 1 |
| 5 | 1 mg/kg AC002911 | Single SQ Injection on Day 1 |
| 6 | 1 mg/kg AC002746 | Single SQ Injection on Day 1 |
| 7 | 1 mg/kg AC002912 | Single SQ Injection on Day 1 |
| 8 | 1 mg/kg AC002913 | Single SQ Injection on Day 1 |
| 9 | 1 mg/kg AC002914 | Single SQ Injection on Day 1 |
| 10 | 1 mg/kg AC002915 | Single SQ Injection on Day 1 |

The lipid-conjugated RNAi agents were designed such that the antisense included a nucleotide sequence complementary to the Adipoq gene transcript, which expresses the protein hormone adiponectin primarily in adipose tissue. Thus, the lipid-conjugated RNAi agents were designed to inhibit the expression of the Adipoq gene. On day 1 prior to dosing, serum was collected. On days 8, 15, and 22 post dose, serum was collected. On day 22 post dosing, animals were sacrificed, adipose tissues were harvested, and the inguinal white adipose tissue (iWAT) and perigonadal white adipose tissue (pgWAT) were collected. Mouse adiponectin in serum was analyzed via enzyme-linked immunoassay (ELISA) assay (R&D Systems, catalog MRP300), normalized pre-dose and control group 1 (Saline). Expression of mouse Adipoq in each tissue was determined using qPCR, with mouse mAr1 as control. Average Adipoq expression for each animal in each tissue was normalized relative to pre-dose and control group 1 (Saline). Results are shown in Table 6A and 6B below.

TABLE 6A
Average relative expression of Adiponectin
in serum (ELISA) in mice of Example 4.

| | Day 1 Pre-dose | | Day 8 | |
|---|---|---|---|---|
| | | Std Dev | | Std Dev |
| Group ID | Avg | (+/−) | Avg | (+/−) |
| 1. Saline | 1.000 | 0.000 | 1.000 | 0.135 |
| 2. 1 mg/kg AC002785 | 1.000 | 0.000 | 0.216 | 0.079 |
| 3. 1 mg/kg AC002873 | 1.000 | 0.000 | 0.136 | 0.022 |
| 4. 1 mg/kg AC002954 | 1.000 | 0.000 | 0.176 | 0.037 |
| 5. 1 mg/kg AC002911 | 1.000 | 0.000 | 0.164 | 0.020 |
| 6. 1 mg/kg AC002746 | 1.000 | 0.000 | 0.245 | 0.071 |
| 7. 1 mg/kg AC002912 | 1.000 | 0.000 | 0.173 | 0.039 |
| 8. 1 mg/kg AC002913 | 1.000 | 0.000 | 0.231 | 0.032 |
| 9. 1 mg/kg AC002914 | 1.000 | 0.000 | 0.303 | 0.039 |
| 10. 1 mg/kg AC002915 | 1.000 | 0.000 | 0.152 | 0.022 |

| | |
|---|---|
| Day 15 | Day 22 |

| | | Std Dev | | Std Dev |
|---|---|---|---|---|
| Group ID | Avg | (+/−) | Avg | (+/−) |
| 1. Saline | 1.000 | 0.094 | 1.000 | 0.176 |
| 2. 1 mg/kg AC002785 | 0.180 | 0.075 | 0.161 | 0.049 |
| 3. 1 mg/kg AC002873 | 0.083 | 0.011 | 0.057 | 0.046 |
| 4. 1 mg/kg AC002954 | 0.091 | 0.035 | 0.153 | 0.055 |
| 5. 1 mg/kg AC002911 | 0.081 | 0.032 | 0.124 | 0.036 |
| 6. 1 mg/kg AC002746 | 0.183 | 0.052 | 0.299 | 0.108 |
| 7. 1 mg/kg AC002912 | 0.132 | 0.051 | 0.188 | 0.089 |
| 8. 1 mg/kg AC002913 | 0.193 | 0.028 | 0.297 | 0.074 |
| 9. 1 mg/kg AC002914 | 0.096 | 0.020 | 0.119 | 0.042 |
| 10. 1 mg/kg AC002915 | 0.130 | 0.043 | 0.188 | 0.041 |

Groups 2-10 showed reduction of Adiponectin in serum at all measured time points. In particular, group 3, animals dosed with an RNAi agent comprising a 5′-terminal LP-413-a and 3′-terminal LP-378-a moiety, showed greater than 90% Adiponectin reduction in serum at days 15 and 22.

TABLE 6B
Average relative expression of Adipoq
(qPCR) in mice, Day 22, of Example 4.

| | mAdipoq (iWAT) | | | mAdipoq (pgWAT) | | |
|---|---|---|---|---|---|---|
| Group ID | Rel Exp | Low | High | Rel Exp | Low | High |
| 1. Saline | 1.000 | 0.149 | 0.175 | 1.000 | 0.091 | 0.100 |
| 2. 1 mg/kg AC002785 | 0.315 | 0.062 | 0.077 | 0.255 | 0.040 | 0.048 |
| 3. 1 mg/kg AC002873 | 0.233 | 0.053 | 0.068 | 0.190 | 0.027 | 0.032 |
| 4. 1 mg/kg AC002954 | 0.332 | 0.097 | 0.136 | 0.195 | 0.067 | 0.101 |
| 5. 1 mg/kg AC002911 | 0.304 | 0.057 | 0.071 | 0.247 | 0.016 | 0.017 |
| 6. 1 mg/kg AC002746 | 0.506 | 0.116 | 0.151 | 0.433 | 0.083 | 0.103 |
| 7. 1 mg/kg AC002912 | 0.330 | 0.165 | 0.328 | 0.300 | 0.097 | 0.144 |
| 8. 1 mg/kg AC002913 | 0.601 | 0.068 | 0.077 | 0.523 | 0.070 | 0.081 |
| 9. 1 mg/kg AC002914 | 0.199 | 0.024 | 0.027 | 0.153 | 0.040 | 0.055 |
| 10. 1 mg/kg AC002915 | 0.368 | 0.104 | 0.146 | 0.233 | 0.083 | 0.130 |

Groups 2-10 showed reduction of Adipoq at day 22 in both collected tissues. In particular, group 3 showed ˜80% reduction in iWAT and pgWAT at day 22.

### Example 5. In Vivo Administration of Lipid-Linked RNAi Agents in Mice

On study day 1, female C7bl/6 mice were injected with either saline or a lipid-conjugated RNAi agent formulated in saline. Five (n=5) animals were dosed in each group at 250 μL/25 g body weight with saline or RNAi agent solution (at 0.25 mg/kg, 0.5 mg/kg, or 1 mg/kg). Animals were injected subcutaneously (SQ) to the dosing regimen of Table 7.

TABLE 7
Dosing regimen for mice of Example 5.

| Group | Dosing Material | Dosing Regimen |
|---|---|---|
| 1 | Saline | Single SQ Injection on Day 1 |
| 2 | 0.25 mg/kg AC002873 | Single SQ Injection on Day 1 |
| 3 | 0.5 mg/kg AC002873 | Single SQ Injection on Day 1 |
| 4 | 1 mg/kg AC002873 | Single SQ Injection on Day 1 |
| 5 | 0.25 mg/kg AC003104 | Single SQ Injection on Day 1 |
| 6 | 0.5 mg/kg AC003104 | Single SQ Injection on Day 1 |
| 7 | 1 mg/kg AC003104 | Single SQ Injection on Day 1 |
| 8 | 0.25 mg/kg AC003105 | Single SQ Injection on Day 1 |
| 9 | 0.5 mg/kg AC003105 | Single SQ Injection on Day 1 |
| 10 | 1 mg/kg AC003105 | Single SQ Injection on Day 1 |
| 11 | 0.25 mg/kg AC003144 | Single SQ Injection on Day 1 |
| 12 | 0.5 mg/kg AC003144 | Single SQ Injection on Day 1 |
| 13 | 1 mg/kg AC003144 | Single SQ Injection on Day 1 |

The lipid-conjugated RNAi agents were designed to inhibit the expression of the Adipoq gene. On day 1 prior to dosing, serum was collected. On days 8, 15, and 22 post dose, serum was collected. On day 22 post dosing, animals were sacrificed, adipose tissues were harvested, and the inguinal white adipose tissue (iWAT) and perigonadal white adipose tissue (pgWAT) were collected. Mouse adiponectin levels in serum were analyzed via enzyme-linked immunoassay (ELISA) assay (R&D Systems, catalog MRP300), normalized pre-dose and control group 1 (Saline). Expression of mouse Adipoq in each tissue was determined using qPCR, with mouse mArl1 as control. Average Adipoq expression for each animal in each tissue was normalized relative to pre-dose and control group 1 (Saline). Results are shown in Table 8A and 8B below.

TABLE 8A
Average relative expression of Adiponectin
in serum (ELISA) in mice of Example 5.

| | Day 1 Pre-dose | | Day 8 | |
|---|---|---|---|---|
| | | Std Dev | | Std Dev |
| Group ID | Avg | (+/−) | Avg | (+/−) |
| 1. Saline | 1.000 | 0.000 | 1.000 | 0.153 |
| 2. 0.25 mg/kg AC002873 | 1.000 | 0.000 | 0.625 | 0.236 |
| 3. 0.5 mg/kg AC002873 | 1.000 | 0.000 | 0.284 | 0.021 |
| 4. 1 mg/kg AC002873 | 1.000 | 0.000 | 0.148 | 0.064 |
| 5. 0.25 mg/kg AC003104 | 1.000 | 0.000 | 0.261 | 0.043 |
| 6. 0.5 mg/kg AC003104 | 1.000 | 0.000 | 0.177 | 0.056 |
| 7. 1 mg/kg AC003104 | 1.000 | 0.000 | 0.108 | 0.017 |
| 8. 0.25 mg/kg AC003105 | 1.000 | 0.000 | 0.297 | 0.028 |
| 9. 0.5 mg/kg AC003105 | 1.000 | 0.000 | 0.413 | 0.072 |
| 10. 1 mg/kg AC003105 | 1.000 | 0.000 | 0.204 | 0.081 |
| 11. 0.25 mg/kg AC003144 | 1.000 | 0.000 | 0.282 | 0.011 |
| 12. 0.5 mg/kg AC003144 | 1.000 | 0.000 | 0.172 | 0.063 |
| 13. 1 mg/kg AC003144 | 1.000 | 0.000 | 0.083 | 0.019 |
| | Day 15 | | Day 22 | |
| | | Std Dev | | Std Dev |
| Group ID | Avg | (+/−) | Avg | (+/−) |
| 1. Saline | 1.000 | 0.355 | 1.000 | 0.140 |
| 2. 0.25 mg/kg AC002873 | 0.473 | 0.157 | 0.673 | 0.210 |
| 3. 0.5 mg/kg AC002873 | 0.176 | 0.025 | 0.230 | 0.041 |
| 4. 1 mg/kg AC002873 | 0.109 | 0.060 | 0.125 | 0.068 |
| 5. 0.25 mg/kg AC003104 | 0.256 | 0.039 | 0.290 | 0.042 |
| 6. 0.5 mg/kg AC003104 | 0.120 | 0.048 | 0.136 | 0.046 |
| 7. 1 mg/kg AC003104 | 0.057 | 0.015 | 0.074 | 0.022 |
| 8. 0.25 mg/kg AC003105 | 0.248 | 0.041 | 0.280 | 0.042 |
| 9. 0.5 mg/kg AC003105 | 0.096 | 0.021 | 0.102 | 0.017 |
| 10. 1 mg/kg AC003105 | 0.066 | 0.012 | 0.071 | 0.019 |
| 11. 0.25 mg/kg AC003144 | 0.154 | 0.017 | 0.217 | 0.093 |
| 12. 0.5 mg/kg AC003144 | 0.088 | 0.020 | 0.101 | 0.061 |
| 13. 1 mg/kg AC003144 | 0.050 | 0.028 | 0.058 | 0.021 |

Groups 2-13 showed reduction of Adiponectin in serum at all time points measured. In particular, group 10, animals dosed with an RNAi agent comprising a 5′-terminal LP-446a and a 3′-terminal LP-378a moiety showed greater than 90% reduction of Adiponectin in serum at days 15 and 22.

TABLE 8B
Average relative expression of Adipoq (qPCR)
in mouse tissues, Day 22, of Example 5.

| | mAdipoq (iWAT) | | | mAdipoq (pgWAT) | | |
|---|---|---|---|---|---|---|
| | Rel | | | Rel | | |
| Group ID | Exp | Low | High | Exp | Low | High |
| 1. Saline | 1.000 | 0.111 | 0.125 | 1.000 | 0.123 | 0.140 |
| 2. 0.25 mg/kg AC002873 | 0.734 | 0.150 | 0.189 | 0.548 | 0.160 | 0.227 |
| 3. 0.5 mg/kg AC002873 | 0.448 | 0.101 | 0.131 | 0.259 | 0.060 | 0.078 |
| 4. 1 mg/kg AC002873 | 0.272 | 0.103 | 0.166 | 0.159 | 0.041 | 0.054 |
| 5. 0.25 mg/kg AC003104 | 0.540 | 0.073 | 0.085 | 0.366 | 0.074 | 0.092 |
| 6. 0.5 mg/kg AC003104 | 0.286 | 0.089 | 0.129 | 0.223 | 0.056 | 0.075 |
| 7. 1 mg/kg AC003104 | 0.149 | 0.030 | 0.038 | 0.191 | 0.051 | 0.069 |
| 8. 0.25 mg/kg AC003105 | 0.554 | 0.133 | 0.175 | 0.304 | 0.060 | 0.074 |
| 9. 0.5 mg/kg AC003105 | 0.282 | 0.037 | 0.043 | 0.274 | 0.038 | 0.043 |
| 10. 1 mg/kg AC003105 | 0.168 | 0.030 | 0.037 | 0.136 | 0.023 | 0.027 |
| 11. 0.25 mg/kg AC003144 | 0.401 | 0.110 | 0.152 | 0.549 | 0.081 | 0.095 |
| 12. 0.5 mg/kg AC003144 | 0.477 | 0.106 | 0.135 | 0.362 | 0.076 | 0.096 |
| 13. 1 mg/kg AC003144 | 0.176 | 0.024 | 0.027 | 0.178 | 0.042 | 0.055 |

Groups 2-13 showed reduction of Adipoq in both tissues. In particular, group 10 showed greater than 80S reduction in Adiponectin in both iWAT and pgWAT at day 22.

### Example 6. In Vivo Administration of Lipid-Linked RNAi Agents in Mice

On study day 1, female C57bl/6 mice were injected with either saline or a lipid-conjugated RNAi agent formulated in saline. Five (n=5) animals were dosed in each group at 250 μL/25 g body weight with saline or RNAi agent solution (at 0.25 mg/kg, 0.5 mg/kg, or 1 mg/kg). Animals were injected subcutaneously (SQ) to the dosing regimen of Table 9.

TABLE 9
Dosing regimen for mice of Example 6.

| Group | Dosing Material | Dosing Regimen |
|---|---|---|
| 1 | Saline | Single SQ Injection on Day 1 |
| 2 | 0.25 mg/kg AC002873 | Single SQ Injection on Day 1 |
| 3 | 0.5 mg/kg AC002873 | Single SQ Injection on Day 1 |
| 4 | 1 mg/kg AC002873 | Single SQ Injection on Day 1 |
| 5 | 0.25 mg/kg AC002914 | Single SQ Injection on Day 1 |
| 6 | 0.5 mg/kg AC002914 | Single SQ Injection on Day 1 |
| 7 | 1 mg/kg AC002914 | Single SQ Injection on Day 1 |
| 8 | 0.25 mg/kg AC003020 | Single SQ Injection on Day 1 |
| 9 | 0.5 mg/kg AC003020 | Single SQ Injection on Day 1 |
| 10 | 1 mg/kg AC003020 | Single SQ Injection on Day 1 |
| 11 | 0.25 mpk mg/kg AC003103 | Single SQ Injection on Day 1 |
| 12 | 0.5 mg/kg AC003103 | Single SQ Injection on Day 1 |
| 13 | 1 mg/kg AC003103 | Single SQ Injection on Day 1 |

The lipid-conjugated RNAi agents were designed to inhibit the expression of the Adipoq gene. On day 1 prior to dosing, serum was collected. On days 8, 15, and 22 post dose, serum was collected. On day 22 post dosing, animals were sacrificed, adipose tissues were harvested, and the inguinal white adipose tissue (iWAT) and perigonadal white adipose tissue (pgWAT) were collected. Mouse adiponectin in serum was analyzed via enzyme-linked immunoassay (ELISA) assay (R&D Systems, catalog MRP300), normalized pre-dose and control group 1 (Saline). Expression of mouse Adipoq in each tissue was determined using qPCR, with mouse mArl1 as control. Average Adipoq expression for each animal in each tissue was normalized relative to pre-dose and control group 1 (Saline). Results are shown in Table 10A and 10B below.

TABLE 10A
Average relative expression of Adiponectin
in serum (ELISA) in mice of Example 6.

| | Day 1 Pre-dose | | Day 8 | |
|---|---|---|---|---|
| | | Std Dev | | Std Dev |
| Group ID | Avg | (+/−) | Avg | (+/−) |
| 1. Saline | 1.000 | 0.000 | 1.000 | 0.137 |
| 2. 0.25 mg/kg AC002873 | 1.000 | 0.000 | 0.657 | 0.157 |
| 3. 0.5 mg/kg AC002873 | 1.000 | 0.000 | 0.344 | 0.101 |
| 4. 1 mg/kg AC002873 | 1.000 | 0.000 | 0.336 | 0.177 |
| 5. 0.25 mg/kg AC002914 | 1.000 | 0.000 | 0.516 | 0.230 |
| 6. 0.5 mg/kg AC002914 | 1.000 | 0.000 | 0.358 | 0.062 |
| 7. 1 mg/kg AC002914 | 1.000 | 0.000 | 0.147 | 0.027 |
| 8. 0.25 mg/kg AC003020 | 1.000 | 0.000 | 0.237 | 0.051 |
| 9. 0.5 mg/kg AC003020 | 1.000 | 0.000 | 0.314 | 0.037 |
| 10. 1 mg/kg AC003020 | 1.000 | 0.000 | 0.158 | 0.063 |
| 11. 0.25 mpk mg/kg AC003103 | 1.000 | 0.000 | 0.752 | 0.278 |
| 12. 0.5 mg/kg AC003103 | 1.000 | 0.000 | 0.356 | 0.111 |
| 13. 1 mg/kg AC003103 | 1.000 | 0.000 | 0.182 | 0.038 |
| | Day 15 | | Day 22 | |
| | | Std Dev | | Std Dev |
| Group ID | Avg | (+/−) | Avg | (+/−) |
| 1. Saline | 1.000 | 0.147 | 1.000 | 0.136 |
| 2. 0.25 mg/kg AC002873 | 0.509 | 0.265 | 0.385 | 0.133 |
| 3. 0.5 mg/kg AC002873 | 0.216 | 0.078 | 0.164 | 0.078 |
| 4. 1 mg/kg AC002873 | 0.406 | 0.246 | 0.230 | 0.160 |
| 5. 0.25 mg/kg AC002914 | 0.515 | 0.327 | 0.411 | 0.245 |
| 6. 0.5 mg/kg AC002914 | 0.234 | 0.033 | 0.180 | 0.011 |
| 7. 1 mg/kg AC002914 | 0.114 | 0.013 | 0.071 | 0.013 |
| 8. 0.25 mg/kg AC003020 | 0.245 | 0.089 | 0.194 | 0.077 |
| 9. 0.5 mg/kg AC003020 | 0.288 | 0.114 | 0.167 | 0.053 |
| 10. 1 mg/kg AC003020 | 0.130 | 0.035 | 0.063 | 0.024 |
| 11. 0.25 mpk mg/kg AC003103 | 0.435 | 0.260 | 0.491 | 0.229 |
| 12. 0.5 mg/kg AC003103 | 0.188 | 0.065 | 0.182 | 0.061 |
| 13. 1 mg/kg AC003103 | 0.080 | 0.044 | 0.070 | 0.015 |

Groups 2-13 showed reduction of Adiponectin in serum. In particular, group 13, animals dosed with an RNAi agent comprising a 5′-terminal LP-208a and a 3′-terminal LP-378a moiety, showed greater than 90% knockdown of Adiponectin at days 15 and 22.

TABLE 10B
Average relative expression of Adipoq
(qPCR) in mice, Day 22, of Example 6.

| | mAdipoq (iWAT) | | | mAdipoq (pgWAT) | | |
|---|---|---|---|---|---|---|
| Group ID | Rel Exp | Low | High | Rel Exp | Low | High |
| 1. Saline | 1.000 | 0.083 | 0.091 | 1.000 | 0.097 | 0.108 |
| 2. 0.25 mg/kg AC002873 | 0.536 | 0.161 | 0.230 | 0.475 | 0.107 | 0.138 |
| 3. 0.5 mg/kg AC002873 | 0.387 | 0.144 | 0.230 | 0.287 | 0.100 | 0.154 |
| 4. 1 mg/kg AC002873 | 0.373 | 0.101 | 0.139 | 0.158 | 0.081 | 0.165 |
| 5. 0.25 mg/kg AC002914 | 0.657 | 0.204 | 0.296 | 0.459 | 0.186 | 0.313 |
| 6. 0.5 mg/kg AC002914 | 0.422 | 0.111 | 0.150 | 0.343 | 0.086 | 0.115 |
| 7. 1 mg/kg AC002914 | 0.233 | 0.067 | 0.094 | 0.294 | 0.078 | 0.106 |
| 8. 0.25 mg/kg AC003020 | 0.497 | 0.093 | 0.115 | 0.468 | 0.092 | 0.114 |
| 9. 0.5 mg/kg AC003020 | 0.448 | 0.055 | 0.063 | 0.422 | 0.058 | 0.067 |
| 10. 1 mg/kg AC003020 | 0.248 | 0.067 | 0.091 | 0.209 | 0.035 | 0.042 |
| 11. 0.25 mpk mg/kg | 0.731 | 0.202 | 0.280 | 0.845 | 0.124 | 0.146 |
| AC003103 | | | | | | |
| 12. 0.5 mg/kg AC003103 | 0.498 | 0.138 | 0.191 | 0.557 | 0.117 | 0.148 |
| 13. 1 mg/kg AC003103 | 0.327 | 0.073 | 0.094 | 0.274 | 0.065 | 0.085 |

Groups 2-13 showed reduction of Adipoq in both tissues. In particular, group 13 showed ˜70% knockdown of Adiponectin in both iWAT and pgWAT at day 22.

### Example 7. In Vivo Administration of Lipid-Linked RNAi Agents in Mice

On study day 1, female C57bl/6 mice were injected with either saline or a lipid-conjugated RNAi agent formulated in saline. Five (n=5) animals were dosed in each group at 250 μL/25 g body weight with saline or RNAi agent solution (at 0.5 mg/kg). Animals were injected subcutaneously (SQ) to the dosing regimen of Table 11.

TABLE 11
Dosing regimen for mice of Example 7.

| Group | Dosing Material | Dosing Regimen |
|---|---|---|
| 1 | Saline | Single SQ Injection on Day 1 |
| 2 | 0.5 mg/kg AC002873 | Single SQ Injection on Day 1 |
| 3 | 0.5 mg/kg AC003058 | Single SQ Injection on Day 1 |
| 4 | 0.5 mg/kg AC003059 | Single SQ Injection on Day 1 |
| 5 | 0.5 mg/kg AC003060 | Single SQ Injection on Day 1 |
| 6 | 0.5 mg/kg AC002571 | Single SQ Injection on Day 1 |
| 7 | 0.5 mg/kg AC003061 | Single SQ Injection on Day 1 |
| 8 | 0.5 mg/kg AC003062 | Single SQ Injection on Day 1 |
| 9 | 0.5 mg/kg AC002649 | Single SQ Injection on Day 1 |
| 10 | 0.5 mg/kg AC003063 | Single SQ Injection on Day 1 |

The lipid-conjugated RNAi agents were designed to inhibit the expression of the Adipoq gene. On day 1 prior to dosing, serum was collected. On days 8, 15, and 22 post dose, serum was collected. On day 22 post dosing, animals were sacrificed, adipose tissues were harvested, and the inguinal white adipose tissue (iWAT) and perigonadal white adipose tissue (pgWAT) were collected. Mouse adiponectin in serum was analyzed via enzyme-linked immunoassay (ELISA) assay (R&D Systems, catalog MRP300), normalized pre-dose and control group 1 (Saline). Expression of mouse Adipoq in each tissue was determined using qPCR, with mouse mAr1 as control. Average Adipoq expression for each animal in each tissue was normalized relative to pre-dose and control group 1 (Saline). Results are shown in Table 12A and 12B below.

TABLE 12A
Average relative expression of Adiponectin
in serum (ELISA) in mice of Example 7.

| | |
|---|---|
| Day 1 Pre-dose | Day 8 |

| | | Std Dev | | Std Dev |
|---|---|---|---|---|
| Group ID | Avg | (+/−) | Avg | (+/−) |
| 1. Saline | 1.000 | 0.000 | 1.000 | 0.099 |
| 2. 0.5 mg/kg AC002873 | 1.000 | 0.000 | 0.282 | 0.084 |
| 3. 0.5 mg/kg AC003058 | 1.000 | 0.000 | 0.286 | 0.062 |
| 4. 0.5 mg/kg AC003059 | 1.000 | 0.000 | 0.235 | 0.093 |
| 5. 0.5 mg/kg AC003060 | 1.000 | 0.000 | 0.388 | 0.129 |
| 6. 0.5 mg/kg AC002571 | 1.000 | 0.000 | 0.899 | 0.243 |
| 7. 0.5 mg/kg AC003061 | 1.000 | 0.000 | 0.461 | 0.088 |
| 8. 0.5 mg/kg AC003062 | 1.000 | 0.000 | 0.335 | 0.058 |
| 9. 0.5 mg/kg AC002649 | 1.000 | 0.000 | 0.559 | 0.226 |
| 10. 0.5 mg/kg AC003063 | 1.000 | 0.000 | 0.521 | 0.224 |

| | |
|---|---|
| Day 15 | Day 22 |

| | | Std Dev | | Std Dev |
|---|---|---|---|---|
| Group ID | Avg | (+/−) | Avg | (+/−) |
| 1. Saline | 1.000 | 0.177 | 1.000 | 0.194 |
| 2. 0.5 mg/kg AC002873 | 0.263 | 0.149 | 0.184 | 0.088 |
| 3. 0.5 mg/kg AC003058 | 0.194 | 0.049 | 0.173 | 0.056 |
| 4. 0.5 mg/kg AC003059 | 0.260 | 0.094 | 0.198 | 0.129 |
| 5. 0.5 mg/kg AC003060 | 0.243 | 0.049 | 0.222 | 0.056 |
| 6. 0.5 mg/kg AC002571 | 0.700 | 0.155 | 0.501 | 0.167 |
| 7. 0.5 mg/kg AC003061 | 0.328 | 0.081 | 0.269 | 0.085 |
| 8. 0.5 mg/kg AC003062 | 0.309 | 0.077 | 0.221 | 0.074 |
| 9. 0.5 mg/kg AC002649 | 0.409 | 0.188 | 0.354 | 0.167 |
| 10. 0.5 mg/kg AC003063 | 0.376 | 0.139 | 0.336 | 0.168 |

Groups 2-10 showed reduction of Adiponectin in serum at all time points. In particular, group 3, animals dosed with an RNAi agent comprising a 5′-terminal LP-455a and a 3′-terminal LP-378a moiety, showed greater than 80% knockdown of Adiponectin at days 15 and 22.

TABLE 12B
Average relative expression of Adipoq
(qPCR) in mice, Day 22, of Example 7.

| | mAdipoq (pgWAT) | | | mAdipoq (iWAT) | | |
|---|---|---|---|---|---|---|
| Group ID | Rel Exp | Low | High | Rel Exp | Low | High |
| 1. Saline | 1.000 | 0.138 | 0.160 | 1.000 | 0.163 | 0.195 |
| 2. 0.5 mg/kg AC002873 | 0.404 | 0.134 | 0.200 | 0.388 | 0.114 | 0.162 |
| 3. 0.5 mg/kg AC003058 | 0.278 | 0.040 | 0.047 | 0.341 | 0.065 | 0.081 |
| 4. 0.5 mg/kg AC003059 | 0.398 | 0.071 | 0.087 | 0.350 | 0.050 | 0.058 |
| 5.0.5 mg/kg AC003060 | 0.489 | 0.083 | 0.101 | 0.350 | 0.059 | 0.070 |
| 6. 0.5 mg/kg AC002571 | 0.849 | 0.156 | 0.191 | 0.706 | 0.121 | 0.145 |
| 7. 0.5 mg/kg AC003061 | 0.462 | 0.116 | 0.155 | 0.558 | 0.040 | 0.043 |
| 8. 0.5 mg/kg AC003062 | 0.458 | 0.079 | 0.095 | 0.464 | 0.051 | 0.058 |
| 9. 0.5 mg/kg AC002649 | 0.407 | 0.135 | 0.202 | 0.453 | 0.109 | 0.144 |
| 10. 0.5 mg/kg AC003063 | 0.529 | 0.173 | 0.258 | 0.430 | 0.166 | 0.270 |

Groups 2-10 showed reduction of Adipoq in both tissues. In particular, group 3 showed ˜70% knockdown of Adiponectin in both pgWAT and iWAT at day 22.

### Example 8. In Vivo Administration of Lipid-Linked RNAi Agents in Mice

On study day 1, female C7bl/6 mice were injected with either saline or a lipid-conjugated RNAi agent formulated in saline. Five (n=5) animals were dosed in each group at 250 μL/25 g body weight with saline or RNAi agent solution (at 0.5 mg/kg or 1 mg/kg). Animals were injected subcutaneously (SQ) to the dosing regimen of Table 13.

TABLE 13
Dosing regimen for mice of Example 8.

| Group | Dosing Material | Dosing Regimen |
|---|---|---|
| 1 | Saline | Single SQ Injection on Day 1 |
| 2 | 0.5 mg/kg AC002873 | Single SQ Injection on Day 1 |
| 3 | 1 mg/kg AC002873 | Single SQ Injection on Day 1 |
| 4 | 0.5 mg/kg AC002954 | Single SQ Injection on Day 1 |
| 5 | 1 mg/kg AC002954 | Single SQ Injection on Day 1 |
| 6 | 0.5 mg/kg AC002911 | Single SQ Injection on Day 1 |
| 7 | 1 mg/kg AC002911 | Single SQ Injection on Day 1 |
| 8 | 0.5 mg/kg AC002912 | Single SQ Injection on Day 1 |
| 9 | 1 mg/kg AC002912 | Single SQ Injection on Day 1 |
| 10 | 0.5 mg/kg AC002914 | Single SQ Injection on Day 1 |
| 11 | 1 mg/kg AC002914 | Single SQ Injection on Day 1 |

The lipid-conjugated RNAi agents were designed to inhibit the expression of the Adipoq gene. On day 1 prior to dosing, serum was collected. On days 8, 15, and 22 post dose, serum was collected. On day 22 post dosing, animals were sacrificed, adipose tissues were harvested, and the inguinal white adipose tissue (iWAT) and perigonadal white adipose tissue (pgWAT) were collected. Mouse adiponectin in serum was analyzed via enzyme-linked immunoassay (ELISA) assay (R&D Systems, catalog MRP300), normalized pre-dose and control group 1 (Saline). Expression of mouse Adipoq in each tissue was determined using qPCR, with mouse mArl1 as control. Average Adipoq expression for each animal in each tissue was normalized relative to pre-dose and control group 1 (Saline). Results are shown in Table 14A and 14B below.

TABLE 14A
Average relative expression of Adiponectin
in serum (ELISA) in mice of Example 8.

| | |
|---|---|
| Day 1 Pre-dose | Day 8 |

| | | Std Dev | | Std Dev |
|---|---|---|---|---|
| Group ID | Avg | (+/−) | Avg | (+/−) |
| 1. Saline | 1.000 | 0.000 | 1.000 | 0.123 |
| 2. 0.5 mg/kg AC002873 | 1.000 | 0.000 | 0.382 | 0.085 |
| 3. 1 mg/kg AC002873 | 1.000 | 0.000 | 0.191 | 0.026 |
| 4. 0.5 mg/kg AC002954 | 1.000 | 0.000 | 0.397 | 0.157 |
| 5. 1 mg/kg AC002954 | 1.000 | 0.000 | 0.195 | 0.025 |
| 6. 0.5 mg/kg AC002911 | 1.000 | 0.000 | 0.277 | 0.036 |
| 7. 1 mg/kg AC002911 | 1.000 | 0.000 | 0.181 | 0.006 |
| 8. 0.5 mg/kg AC002912 | 1.000 | 0.000 | 0.317 | 0.067 |
| 9. 1 mg/kg AC002912 | 1.000 | 0.000 | 0.278 | 0.055 |
| 10. 0.5 mg/kg AC002914 | 1.000 | 0.000 | 0.299 | 0.097 |
| 11. 1 mg/kg AC002914 | 1.000 | 0.000 | 0.286 | 0.039 |

| | |
|---|---|
| Day 15 | Day 22 |

| | | Std Dev | | Std Dev |
|---|---|---|---|---|
| Group ID | Avg | (+/−) | Avg | (+/−) |
| 1. Saline | 1.000 | 0.104 | 1.000 | 0.148 |
| 2. 0.5 mg/kg AC002873 | 0.263 | 0.079 | 0.210 | 0.053 |
| 3. 1 mg/kg AC002873 | 0.111 | 0.022 | 0.083 | 0.016 |
| 4. 0.5 mg/kg AC002954 | 0.310 | 0.033 | 0.221 | 0.021 |
| 5. 1 mg/kg AC002954 | 0.157 | 0.028 | 0.108 | 0.018 |
| 6. 0.5 mg/kg AC002911 | 0.235 | 0.061 | 0.171 | 0.043 |
| 7. 1 mg/kg AC002911 | 0.185 | 0.013 | 0.122 | 0.012 |
| 8. 0.5 mg/kg AC002912 | 0.225 | 0.048 | 0.174 | 0.045 |
| 9. 1 mg/kg AC002912 | 0.096 | 0.007 | 0.106 | 0.011 |
| 10. 0.5 mg/kg AC002914 | 0.128 | 0.025 | 0.158 | 0.042 |
| 11. 1 mg/kg AC002914 | 0.130 | 0.034 | 0.125 | 0.011 |

Groups 2-11 showed reduction of Adiponectin in serum at all time points measured. In particular, groups 3 and 9, animals dosed with an RNAi agent comprising a 5′-terminal LP-413a and a 3′-terminal LP-378a moiety, and a 5′-terminal LP-426a and a 3′-terminal LP-378a moiety, respectively, showed ˜90% knockdown of Adiponectin at days 15 and 22.

TABLE 14B
Average relative expression of Adipoq
(qPCR) in mice, Day 22, of Example 8.

| | mAdipoq (iWAT) | | | mAdipoq (pgWAT) | | |
|---|---|---|---|---|---|---|
| Group ID | Rel Exp | Low | High | Rel Exp | Low | High |
| 1. Saline | 1.000 | 0.309 | 0.448 | 1.000 | 0.259 | 0.350 |
| 2. 0.5 mg/kg AC002873 | 0.280 | 0.061 | 0.078 | 0.579 | 0.065 | 0.073 |
| 3. 1 mg/kg AC002873 | 0.154 | 0.066 | 0.115 | 0.212 | 0.104 | 0.205 |
| 4. 0.5 mg/kg AC002954 | 0.357 | 0.047 | 0.054 | 0.547 | 0.087 | 0.103 |
| 5. 1 mg/kg AC002954 | 0.225 | 0.051 | 0.065 | 0.403 | 0.094 | 0.123 |
| 6. 0.5 mg/kg AC002911 | 0.361 | 0.063 | 0.077 | 0.605 | 0.108 | 0.131 |
| 7. 1 mg/kg AC002911 | 0.241 | 0.054 | 0.069 | 0.436 | 0.079 | 0.097 |
| 8. 0.5 mg/kg AC002912 | 0.404 | 0.079 | 0.099 | 0.635 | 0.118 | 0.145 |
| 9. 1 mg/kg AC002912 | 0.176 | 0.078 | 0.141 | 0.362 | 0.149 | 0.254 |
| 10. 0.5 mg/kg AC002914 | 0.268 | 0.045 | 0.055 | 0.493 | 0.106 | 0.135 |
| 11. 1 mg/kg AC002914 | 0.241 | 0.043 | 0.053 | 0.320 | 0.049 | 0.057 |

Groups 2-11 showed reduction of Adipoq in both tissues. In particular, groups 3 and 9 both showed greater than 80% knockdown of Adiponectin in iWAT, and greater than 60% knockdown of Adiponectin in pgWAT.

### Example 9. In Vivo Administration of Lipid-Linked RNAi Agents in Mice

On study day 1, female C57bl/6 mice were injected with either saline or a lipid-conjugated RNAi agent formulated in saline. Five (n=5) animals were dosed in each group at 250 μL/25 g body weight with saline or RNAi agent solution (at 0.5 mg/kg). Animals were injected subcutaneously (SQ) to the dosing regimen of Table 15.

TABLE 15
Dosing regimen for mice of Example 9.

| Group | Dosing Material | Dosing Regimen |
|---|---|---|
| 1 | Saline | Single SQ Injection on Day 1 |
| 2 | 0.5 mg/kg AC002873 | Single SQ Injection on Day 1 |
| 3 | 0.5 mg/kg AC003018 | Single SQ Injection on Day 1 |
| 4 | 0.5 mg/kg AC003019 | Single SQ Injection on Day 1 |
| 5 | 0.5 mg/kg AC003020 | Single SQ Injection on Day 1 |
| 6 | 0.5 mg/kg AC003021 | Single SQ Injection on Day 1 |
| 7 | 0.5 mg/kg AC002571 | Single SQ Injection on Day 1 |
| 8 | 0.5 mg/kg AC003022 | Single SQ Injection on Day 1 |
| 9 | 0.5 mg/kg AC003023 | Single SQ Injection on Day 1 |
| 10 | 0.5 mg/kg AC003024 | Single SQ Injection on Day 1 |
| 11 | 0.5 mg/kg AC003025 | Single SQ Injection on Day 1 |
| 12 | 0.5 mg/kg AC003031 | Single SQ Injection on Day 1 |

On day 1 prior to dosing, serum was collected. On days 8, 15, and 22 post dose, serum was collected. On day 22 post dosing, animals were sacrificed, adipose tissues were harvested, and the inguinal white adipose tissue (iWAT) and perigonadal white adipose tissue (pgWAT) were collected. Mouse adiponectin in serum was analyzed via enzyme-linked immunoassay (ELISA) assay (R&D Systems, catalog MRP300), normalized pre-dose and control group 1 (Saline). Expression of mouse Adipoq in each tissue was determined using qPCR, with mouse mArl1 as control. Average Adipoq expression for each animal in each tissue was normalized relative to pre-dose and control group 1 (Saline). Results are shown in Table 16A and 16B below.

TABLE 16A
Average relative expression of Adiponectin
in serum (ELISA) in mice of Example 9.

| | |
|---|---|
| Day 1 Pre-dose | Day 8 |

| | | Std Dev | | Std Dev |
|---|---|---|---|---|
| Group ID | Avg | (+/−) | Avg | (+/−) |
| 1. Saline | 1.000 | 0.000 | 1.000 | 0.117 |
| 2. 0.5 mg/kg AC002873 | 1.000 | 0.000 | 0.416 | 0.055 |
| 3. 0.5 mg/kg AC003018 | 1.000 | 0.000 | 0.580 | 0.038 |
| 4. 0.5 mg/kg AC003019 | 1.000 | 0.000 | 0.582 | 0.090 |
| 5. 0.5 mg/kg AC003020 | 1.000 | 0.000 | 0.327 | 0.113 |
| 6. 0.5 mg/kg AC003021 | 1.000 | 0.000 | 0.371 | 0.097 |
| 7. 0.5 mg/kg AC002571 | 1.000 | 0.000 | 0.410 | 0.102 |
| 8. 0.5 mg/kg AC003022 | 1.000 | 0.000 | 1.041 | 0.151 |
| 9. 0.5 mg/kg AC003023 | 1.000 | 0.000 | 0.711 | 0.218 |
| 10. 0.5 mg/kg AC003024 | 1.000 | 0.000 | 0.536 | 0.108 |
| 11. 0.5 mg/kg AC003025 | 1.000 | 0.000 | 0.791 | 0.071 |
| 12. 0.5 mg/kg AC003031 | 1.000 | 0.000 | 0.564 | 0.105 |

| | |
|---|---|
| Day 15 | Day 22 |

| | | Std Dev | | Std Dev |
|---|---|---|---|---|
| Group ID | Avg¶ | (+/−) | Avg | (+/−) |
| 1. Saline | 1.000 | 0.156 | 1.000 | 0.183 |
| 2. 0.5 mg/kg AC002873 | 0.331 | 0.087 | 0.247 | 0.172 |
| 3. 0.5 mg/kg AC003018 | 0.838 | 0.084 | 0.558 | 0.089 |
| 4. 0.5 mg/kg AC003019 | 0.430 | 0.052 | 0.407 | 0.094 |
| 5. 0.5 mg/kg AC003020 | 0.228 | 0.104 | 0.258 | 0.092 |
| 6. 0.5 mg/kg AC003021 | 0.215 | 0.071 | 0.253 | 0.117 |
| 7. 0.5 mg/kg AC002571 | 0.354 | 0.092 | 0.411 | 0.187 |
| 8. 0.5 mg/kg AC003022 | 1.124 | 0.274 | 1.421 | 0.402 |
| 9. 0.5 mg/kg AC003023 | 0.660 | 0.092 | 0.626 | 0.169 |
| 10. 0.5 mg/kg AC003024 | 0.476 | 0.134 | 0.386 | 0.083 |
| 11. 0.5 mg/kg AC003025 | 1.212 | 0.161 | 0.558 | 0.164 |
| 12. 0.5 mg/kg AC003031 | 0.794 | 0.356 | 0.626 | 0.210 |

Groups 2-7 and 9-12 showed reduction of Adiponectin at Day 8. Groups 2-7, 9, 10, and 12 showed reduction of Adiponectin at Day 15. Groups 2-7 and 9-12 showed reduction of Adiponectin at Day 22.

TABLE 16B
Average relative expression of Adipoq
(qPCR) in mice, Day 22, of Example 9.

| | mAdipoq (iWAT) | | | mAdipoq (pgWAT) | | |
|---|---|---|---|---|---|---|
| Group ID | Rel Exp | Low | High | Rel Exp | Low | High |
| 1. Saline | 1.000 | 0.126 | 0.144 | 1.000 | 0.169 | 0.203 |
| 2. 0.5 mg/kg AC002873 | 0.481 | 0.094 | 0.116 | 0.376 | 0.073 | 0.090 |
| 3. 0.5 mg/kg AC003018 | 0.864 | 0.184 | 0.233 | 0.692 | 0.116 | 0.139 |
| 4. 0.5 mg/kg AC003019 | 0.647 | 0.149 | 0.194 | 0.586 | 0.122 | 0.155 |
| 5. 0.5 mg/kg AC003020 | 0.379 | 0.096 | 0.128 | 0.326 | 0.067 | 0.084 |
| 6. 0.5 mg/kg AC003021 | 0.407 | 0.050 | 0.057 | 0.325 | 0.084 | 0.112 |
| 7. 0.5 mg/kg AC002571 | 0.686 | 0.106 | 0.125 | 0.558 | 0.080 | 0.094 |
| 8. 0.5 mg/kg AC003022 | 1.088 | 0.155 | 0.180 | 1.076 | 0.093 | 0.102 |
| 9. 0.5 mg/kg AC003023 | 0.719 | 0.117 | 0.139 | 0.602 | 0.115 | 0.142 |
| 10. 0.5 mg/kg AC003024 | 0.500 | 0.088 | 0.107 | 0.496 | 0.067 | 0.077 |
| 11. 0.5 mg/kg AC003025 | 0.767 | 0.066 | 0.072 | 0.599 | 0.247 | 0.419 |
| 12. 0.5 mg/kg AC003031 | 0.762 | 0.104 | 0.121 | 0.606 | 0.062 | 0.069 |

Groups 2-7 and 9-12 showed reduction of Adipoq in both tissues. In particular, groups 5 and 6 showed ˜62% and ˜59% knockdown, respectively, of Adiponectin in iWAT and ˜67. knockdown of Adiponectin in pgWAT.

### Example 10. In Vivo Administration of Lipid-Linked RNAi Agents in Mice

On study day 1, female C7bl/6 mice were injected with either saline or a lipid-conjugated RNAi agent formulated in saline. Five (n=5) animals were dosed in each group at 250 μL/25 g body weight with saline or RNAi agent solution (at 0.75 mg/kg, 1.5 mg/kg, or 3 mg/kg). Animals were injected subcutaneously (SQ) to the dosing regimen of Table 17.

TABLE 17
Dosing regimen for mice of Example 10.

| Group | Dosing Material | Dosing Regimen |
|---|---|---|
| 1 | Saline | Single SQ Injection on Day 1 |
| 2 | 0.75 mg/kg AC002785 | Single SQ Injection on Day 1 |
| 3 | 1.5 mg/kg AC002785 | Single SQ Injection on Day 1 |
| 4 | 3 mg/kg AC002785 | Single SQ Injection on Day 1 |
| 5 | 0.75 mg/kg AC002744 | Single SQ Injection on Day 1 |
| 6 | 1.5 mg/kg AC002744 | Single SQ Injection on Day 1 |
| 7 | 3 mg/kg AC002744 | Single SQ Injection on Day 1 |
| 8 | 0.75 mg/kg AC002873 | Single SQ Injection on Day 1 |
| 9 | 1.5 mg/kg AC002873 | Single SQ Injection on Day 1 |
| 10 | 3 mg/kg AC002873 | Single SQ Injection on Day 1 |
| 11 | 0.75 mg/kg AC002874 | Single SQ Injection on Day 1 |
| 12 | 1.5 mg/kg AC002874 | Single SQ Injection on Day 1 |
| 13 | 3 mg/kg AC002874 | Single SQ Injection on Day 1 |

The lipid-conjugated RNAi agents were designed to inhibit the expression of the Adipoq gene. On day 1 prior to dosing, serum was collected. On days 8, 15, and 22 post dose, serum was collected. On day 22 post dosing, animals were sacrificed, adipose tissues were harvested, and the inguinal white adipose tissue (iWAT) and perigonadal white adipose tissue (pgWAT) were collected. Mouse adiponectin in serum was analyzed via enzyme-linked immunoassay (ELISA) assay (R&D Systems, catalog MRP300), normalized pre-dose and control group 1 (Saline). Expression of mouse Adipoq in each tissue was determined using qPCR, with mouse mArl1 as control. Average Adipoq expression for each animal in each tissue was normalized relative to pre-dose and control group 1 (Saline). Results are shown in Table 18A and 18B below.

TABLE 18A
Average relative expression of Adiponectin
in serum (ELISA) in mice of Example 10.

| | Day 1 Pre-dose | | Day 8 | |
|---|---|---|---|---|
| | | Std Dev | | Std Dev |
| Group ID | Avg | (+/−) | Avg | (+/−) |
| 1. Saline | 1.000 | 0.000 | 1.000 | 0.147 |
| 2. 0.75 mg/kg AC002785 | 1.000 | 0.000 | 0.280 | 0.057 |
| 3. 1.5 mg/kg AC002785 | 1.000 | 0.000 | 0.195 | 0.069 |
| 4. 3 mg/kg AC002785 | 1.000 | 0.000 | 0.084 | 0.031 |
| 5. 0.75 mg/kg AC002744 | 1.000 | 0.000 | 0.171 | 0.074 |
| 6. 1.5 mg/kg AC002744 | 1.000 | 0.000 | 0.108 | 0.024 |
| 7. 3 mg/kg AC002744 | 1.000 | 0.000 | 0.065 | 0.016 |
| 8. 0.75 mg/kg AC002873 | 1.000 | 0.000 | 0.140 | 0.042 |
| 9. 1.5 mg/kg AC002873 | 1.000 | 0.000 | 0.111 | 0.019 |
| 10. 3 mg/kg AC002873 | 1.000 | 0.000 | 0.051 | 0.011 |
| 11. 0.75 mg/kg AC002874 | 1.000 | 0.000 | 0.248 | 0.093 |
| 12. 1.5 mg/kg AC002874 | 1.000 | 0.000 | 0.129 | 0.036 |
| 13. 3 mg/kg AC002874 | 1.000 | 0.000 | 0.077 | 0.017 |
| | Day 15 | | Day 22 | |
| | | Std Dev | | Std Dev |
| Group ID | Avg | (+/−) | Avg | (+/−) |
| 1. Saline | 1.000 | 0.208 | 1.000 | 0.227 |
| 2. 0.75 mg/kg AC002785 | 0.151 | 0.036 | 0.136 | 0.040 |
| 3. 1.5 mg/kg AC002785 | 0.085 | 0.010 | 0.071 | 0.008 |
| 4. 3 mg/kg AC002785 | 0.032 | 0.007 | 0.033 | 0.004 |
| 5. 0.75 mg/kg AC002744 | 0.074 | 0.015 | 0.071 | 0.015 |
| 6. 1.5 mg/kg AC002744 | 0.056 | 0.005 | 0.043 | 0.005 |
| 7. 3 mg/kg AC002744 | 0.030 | 0.004 | 0.025 | 0.002 |
| 8. 0.75 mg/kg AC002873 | 0.076 | 0.028 | 0.070 | 0.025 |
| 9. 1.5 mg/kg AC002873 | 0.035 | 0.005 | 0.040 | 0.007 |
| 10. 3 mg/kg AC002873 | 0.022 | 0.003 | 0.023 | 0.004 |
| 11. 0.75 mg/kg AC002874 | 0.072 | 0.014 | 0.084 | 0.016 |
| 12. 1.5 mg/kg AC002874 | 0.052 | 0.012 | 0.056 | 0.013 |
| 13. 3 mg/kg AC002874 | 0.030 | 0.004 | 0.032 | 0.004 |

Groups 2-13 showed reduction of Adiponectin in serum at all time points measured. All groups showed ˜85% or greater knockdown at days 15 and 22.

TABLE 18B
Average relative expression of Adipoq
(qPCR) in mice, Day 22, of Example 10.

| | mAdipoq (iWAT) | | | mAdipoq (pgWAT) | | |
|---|---|---|---|---|---|---|
| | Rel | | | Rel | | |
| Group ID | Exp | Low | High | Exp | Low | High |
| 1. Saline | 1.000 | 0.239 | 0.315 | 1.000 | 0.161 | 0.192 |
| 2. 0.75 mg/kg AC002785 | 0.465 | 0.113 | 0.150 | 0.312 | 0.058 | 0.071 |
| 3. 1.5 mg/kg AC002785 | 0.182 | 0.043 | 0.056 | 0.231 | 0.033 | 0.039 |
| 4. 3 mg/kg AC002785 | 0.180 | 0.047 | 0.063 | 0.130 | 0.021 | 0.025 |
| 5. 0.75 mg/kg AC002744 | 0.240 | 0.050 | 0.063 | 0.176 | 0.047 | 0.064 |
| 6. 1.5 mg/kg AC002744 | 0.230 | 0.024 | 0.026 | 0.130 | 0.033 | 0.044 |
| 7. 3 mg/kg AC002744 | 0.093 | 0.025 | 0.034 | 0.054 | 0.013 | 0.017 |
| 8. 0.75 mg/kg AC002873 | 0.193 | 0.051 | 0.068 | 0.201 | 0.027 | 0.031 |
| 9. 1.5 mg/kg AC002873 | 0.146 | 0.020 | 0.023 | 0.101 | 0.032 | 0.046 |
| 10. 3 mg/kg AC002873 | 0.063 | 0.028 | 0.049 | 0.038 | 0.014 | 0.022 |
| 11. 0.75 mg/kg AC002874 | 0.312 | 0.043 | 0.050 | 0.259 | 0.082 | 0.120 |
| 12. 1.5 mg/kg AC002874 | 0.227 | 0.029 | 0.034 | 0.159 | 0.022 | 0.026 |
| 13. 3 mg/kg AC002874 | 0.134 | 0.021 | 0.024 | 0.085 | 0.014 | 0.017 |

Groups 2-13 showed reduction of Adipoq in both tissues. In particular, groups 7 and 10, animals that were dosed with an RNAi agent comprising a 5′-terminal LP-18a and a 3′-terminal LP-378a moiety, and a 5′-terminal LP-413a and a 3′-terminal LP-378a moiety, respectively (at 3 mg/kg), showed greater than 90% knockdown of Adiponectin in both iWAT and pgWAT.

### Example 11. In Vivo Administration of Lipid-Linked RNAi Agents in Mice

On study day 1, female C57bl/6 mice were injected with either saline or a lipid-conjugated RNAi agent formulated in saline. Five (n=5) animals were dosed in each group at 250 μL/25 g body weight with saline or RNAi agent solution (at 1 mg/kg). Animals were injected subcutaneously (SQ) to the dosing regimen of Table 19.

TABLE 19
Dosing regimen for mice of Example 11.

| Group | Dosing Material | Dosing Regimen |
|---|---|---|
| 1 | Saline | Single SQ Injection on Day 1 |
| 2 | 1 mg/kg AC002772 | Single SQ Injection on Day 1 |
| 3 | 1 mg/kg AC002571 | Single SQ Injection on Day 1 |
| 4 | 1 mg/kg AC002773 | Single SQ Injection on Day 1 |
| 5 | 1 mg/kg AC002795 | Single SQ Injection on Day 1 |
| 6 | 1 mg/kg AC002796 | Single SQ Injection on Day 1 |
| 7 | 1 mg/kg AC002577 | Single SQ Injection on Day 1 |
| 8 | 1 mg/kg AC002679 | Single SQ Injection on Day 1 |
| 9 | 1 mg/kg AC002797 | Single SQ Injection on Day 1 |
| 10 | 1 mg/kg AC002785 | Single SQ Injection on Day 1 |
| 11 | 1 mg/kg AC002788 | Single SQ Injection on Day 1 |

The lipid-conjugated RNAi agents were designed to inhibit the expression of the Adipoq gene. On day 1 prior to dosing, serum was collected. On days 8, 15, and 22 post dose, serum was collected. On day 22 post dosing, animals were sacrificed, adipose tissues were harvested, and the inguinal white adipose tissue (iWAT) and perigonadal white adipose tissue (pgWAT) were collected. Mouse adiponectin in serum was analyzed via enzyme-linked immunoassay (ELISA) assay (R&D Systems, catalog MRP300), normalized pre-dose and control group 1 (Saline). Expression of mouse Adipoq in each tissue was determined using qPCR, with mouse mArl1 as control. Average Adipoq expression for each animal in each tissue was normalized relative to pre-dose and control group 1 (Saline). Results are shown in Table 20A and 20B below.

TABLE 20A
Average relative expression of Adiponectin
in serum (ELISA) in mice of Example 11.

| | |
|---|---|
| Day 1 Pre-dose | Day 8 |

| | | Std Dev | | Std Dev |
|---|---|---|---|---|
| Group ID | Avg | (+/−) | Avg | (+/−) |
| 1. Saline | 1.000 | 0.000 | 1.000 | 0.132 |
| 2. 1 mg/kg AC002772 | 1.000 | 0.000 | 0.950 | 0.103 |
| 3. 1 mg/kg AC002571 | 1.000 | 0.000 | 0.327 | 0.231 |
| 4. 1 mg/kg AC002773 | 1.000 | 0.000 | 0.678 | 0.106 |
| 5. 1 mg/kg AC002795 | 1.000 | 0.000 | 0.542 | 0.111 |
| 6. 1 mg/kg AC002796 | 1.000 | 0.000 | 0.183 | 0.020 |
| 7. 1 mg/kg AC002577 | 1.000 | 0.000 | 0.234 | 0.020 |
| 8. 1 mg/kg AC002679 | 1.000 | 0.000 | 0.401 | 0.097 |
| 9. 1 mg/kg AC002797 | 1.000 | 0.000 | 0.365 | 0.124 |
| 10. 1 mg/kg AC002785 | 1.000 | 0.000 | 0.209 | 0.061 |
| 11. 1 mg/kg AC002788 | 1.000 | 0.000 | 0.436 | 0.107 |

| | |
|---|---|
| Day 15 | Day 22 |

| | | Std Dev | | Std Dev |
|---|---|---|---|---|
| Group ID | Avg | (+/−) | Avg | (+/−) |
| 1. Saline | 1.000 | 0.082 | 1.000 | 0.167 |
| 2. 1 mg/kg AC002772 | 0.955 | 0.189 | 0.840 | 0.096 |
| 3. 1 mg/kg AC002571 | 0.363 | 0.263 | 0.332 | 0.251 |
| 4. 1 mg/kg AC002773 | 0.562 | 0.233 | 0.454 | 0.204 |
| 5. 1 mg/kg AC002795 | 0.486 | 0.051 | 0.370 | 0.067 |
| 6. 1 mg/kg AC002796 | 0.119 | 0.016 | 0.111 | 0.017 |
| 7. 1 mg/kg AC002577 | 0.162 | 0.024 | 0.140 | 0.036 |
| 8. 1 mg/kg AC002679 | 0.370 | 0.079 | 0.333 | 0.080 |
| 9. 1 mg/kg AC002797 | 0.300 | 0.104 | 0.280 | 0.104 |
| 10. 1 mg/kg AC002785 | 0.124 | 0.038 | 0.113 | 0.036 |
| 11. 1 mg/kg AC002788 | 0.353 | 0.109 | 0.345 | 0.097 |

Groups 2-11 showed reduction of Adiponectin in serum at all time points measured. Group 2, which comprised mice administered RNAi agents comprising an NEM cap instead of a lipid, showed only a modest reduction of Adiponectin in serum, compared to greater reduction in other groups where the RNAi agent comprised at least one lipid at the 5′ or 3′ terminal nucleotide. In particular, group 7, animals dosed with an RNAi agent comprising a 5′-terminal LP-379a and a 3′-terminal LP-378a moiety, showed greater than 85% knockdown of Adiponectin in serum at days 15 and 22.

TABLE 20B
Average relative expression of Adipoq
(qPCR) in mice, Day 22, of Example 11.

| | mAdipoq (iWAT) | | | mAdipoq (pgWAT) | | |
|---|---|---|---|---|---|---|
| Group ID | Rel Exp | Low | High | Rel Exp | Low | High |
| 1. Saline | 1.000 | 0.160 | 0.191 | 1.000 | 0.199 | 0.249 |
| 2. 1 mg/kg AC002772 | 1.056 | 0.203 | 0.252 | 1.073 | 0.186 | 0.225 |
| 3. 1 mg/kg AC002571 | 0.654 | 0.243 | 0.385 | 0.540 | 0.201 | 0.321 |
| 4. 1 mg/kg AC002773 | 1.102 | 0.311 | 0.433 | 0.648 | 0.223 | 0.339 |
| 5. 1 mg/kg AC002795 | 0.894 | 0.266 | 0.379 | 0.849 | 0.173 | 0.217 |
| 6. 1 mg/kg AC002796 | 0.751 | 0.298 | 0.494 | 0.285 | 0.063 | 0.081 |
| 7. 1 mg/kg AC002577 | 0.758 | 0.289 | 0.466 | 0.300 | 0.067 | 0.086 |
| 8. 1 mg/kg AC002679 | 1.172 | 0.235 | 0.295 | 0.792 | 0.118 | 0.139 |
| 9. 1 mg/kg AC002797 | 0.833 | 0.296 | 0.459 | 0.479 | 0.095 | 0.119 |
| 10. 1 mg/kg AC002785 | 0.448 | 0.145 | 0.213 | 0.291 | 0.041 | 0.048 |
| 11. 1 mg/kg AC002788 | 0.886 | 0.392 | 0.702 | 0.808 | 0.147 | 0.179 |

Groups 3, 5-7, and 9-11 showed reduction of Adipoq in iWAT. Groups 3-11 showed reduction of Adipoq in pgWAT. Group 2, which comprised mice administered RNAi agents comprising an NEM cap instead of a lipid, showed a slight increase in expression of Adipoq in tissue, compared with greater reduction in other groups where the RNAi agent comprised at least one lipid at the 5′ or 3′ terminal nucleotide.

### Example 12. In Vivo Administration of Lipid-Linked RNAi Agents in Mice

On study day 1, female C57bl/6 mice were injected with either saline or RNAi agent formulated in saline. Five (n=5) (for Groups 1-4 and Groups 6-11) or four (n=4) (for Group 5) animals were dosed in each group at 250 μL/25 g body weight with saline or RNAi agent solution (at 1 mg/kg). Animals were injected subcutaneously (SQ) to the dosing regimen of Table 21.

TABLE 21
Dosing regimen for mice of Example 12.

| Group | Dosing Material | Dosing Regimen |
|---|---|---|
| 1 | Saline | Single SQ Injection on Day 1 |
| 2 | 1 mg/kg AC002571 | Single SQ Injection on Day 1 |
| 3 | 1 mg/kg AC002739 | Single SQ Injection on Day 1 |
| 4 | 1 mg/kg AC002740 | Single SQ Injection on Day 1 |
| 5 | 1 mg/kg AC002650 | Single SQ Injection on Day 1 |
| 6 | 1 mg/kg AC002785 | Single SQ Injection on Day 1 |
| 7 | 1 mg/kg AC002786 | Single SQ Injection on Day 1 |
| 8 | 1 mg/kg AC002787 | Single SQ Injection on Day 1 |
| 9 | 1 mg/kg AC002743 | Single SQ Injection on Day 1 |
| 10 | 1 mg/kg AC002744 | Single SQ Injection on Day 1 |
| 11 | 1 mg/kg AC002745 | Single SQ Injection on Day 1 |

On day 1 prior to dosing, serum was collected. On days 8, 15, and 22 post dose, serum was collected. On day 22 post dosing, animals were sacrificed, adipose tissues were harvested, and the inguinal white adipose tissue (iWAT) and perigonadal white adipose tissue (pgWAT) were collected. Mouse adiponectin in serum was analyzed via enzyme-linked immunoassay (ELISA) assay (R&D Systems, catalog MRP300), normalized pre-dose and control group 1 (Saline). Expression of mouse Adipoq in each tissue was determined using qPCR, with mouse mAr1 as control. Average Adipoq expression for each animal in each tissue was normalized relative to pre-dose and control group 1 (Saline). Results are shown in Table 22A and 22B below.

TABLE 22A
Average relative expression of Adiponectin
in serum (ELISA) in mice of Example 12.

| | |
|---|---|
| Day 1 Pre-dose | Day 8 |

| | | Std Dev | | Std Dev |
|---|---|---|---|---|
| Group ID | Avg | (+/−) | Avg | (+/−) |
| 1. Saline | 1.000 | 0.000 | 1.000 | 0.108 |
| 2. 1 mg/kg AC002571 | 1.000 | 0.000 | 0.325 | 0.123 |
| 3. 1 mg/kg AC002739 | 1.000 | 0.000 | 0.453 | 0.085 |
| 4. 1 mg/kg AC002740 | 1.000 | 0.000 | 0.253 | 0.040 |
| 5. 1 mg/kg AC002650 | 1.000 | 0.000 | 0.260 | 0.077 |
| 6. 1 mg/kg AC002785 | 1.000 | 0.000 | 0.172 | 0.028 |
| 7. 1 mg/kg AC002786 | 1.000 | 0.000 | 0.291 | 0.075 |
| 8. 1 mg/kg AC002787 | 1.000 | 0.000 | 0.325 | 0.066 |
| 9. 1 mg/kg AC002743 | 1.000 | 0.000 | 0.424 | 0.125 |
| 10. 1 mg/kg AC002744 | 1.000 | 0.000 | 0.150 | 0.020 |
| 11. 1 mg/kg AC002745 | 1.000 | 0.000 | 0.276 | 0.051 |

| | |
|---|---|
| Day 15 | Day 22 |

| | | Std Dev | | Std Dev |
|---|---|---|---|---|
| Group ID | Avg | (+/−) | Avg | (+/−) |
| 1. Saline | 1.000 | 0.169 | 1.000 | 0.068 |
| 2. 1 mg/kg AC002571 | 0.221 | 0.068 | 0.237 | 0.068 |
| 3. 1 mg/kg AC002739 | 0.337 | 0.125 | 0.390 | 0.117 |
| 4. 1 mg/kg AC002740 | 0.202 | 0.035 | 0.229 | 0.046 |
| 5. 1 mg/kg AC002650 | 0.200 | 0.069 | 0.194 | 0.045 |
| 6. 1 mg/kg AC002785 | 0.128 | 0.020 | 0.121 | 0.024 |
| 7. 1 mg/kg AC002786 | 0.200 | 0.060 | 0.195 | 0.044 |
| 8. 1 mg/kg AC002787 | 0.282 | 0.037 | 0.319 | 0.065 |
| 9. 1 mg/kg AC002743 | 0.281 | 0.208 | 0.426 | 0.133 |
| 10. 1 mg/kg AC002744 | 0.104 | 0.006 | 0.110 | 0.006 |
| 11. 1 mg/kg AC002745 | 0.234 | 0.044 | 0.224 | 0.032 |

Groups 2-11 showed reduction of Adiponectin in serum at all time points measured. In particular, group 10, which comprised animals dosed with an RNAi agent comprising a 5′-terminal LP-128a and a 3′-terminal LP-378a moiety, showed greater than 85% knockdown of Adiponectin in serum at days 15 and 22.

TABLE 22B
Average relative expression of Adipoq
(qPCR) in mice, Day 22, of Example 12.

| | mAdipoq (iWAT) | | | mAdipoq (pgWAT) | | |
|---|---|---|---|---|---|---|
| Group ID | Rel Exp | Low | High | Rel Exp | Low | High |
| 1. Saline | 1.000 | 0.149 | 0.176 | 1.000 | 0.114 | 0.129 |
| 2. 1 mg/kg AC002571 | 0.551 | 0.138 | 0.184 | 0.477 | 0.164 | 0.250 |
| 3. 1 mg/kg AC002739 | 0.679 | 0.159 | 0.208 | 0.514 | 0.114 | 0.147 |
| 4. 1 mg/kg AC002740 | 0.408 | 0.046 | 0.052 | 0.344 | 0.064 | 0.078 |
| 5. 1 mg/kg AC002650 | 0.419 | 0.087 | 0.109 | 0.263 | 0.054 | 0.067 |
| 6. 1 mg/kg AC002785 | 0.339 | 0.031 | 0.034 | 0.276 | 0.044 | 0.052 |
| 7. 1 mg/kg AC002786 | 0.512 | 0.097 | 0.119 | 0.386 | 0.084 | 0.107 |
| 8. 1 mg/kg AC002787 | 0.598 | 0.125 | 0.158 | 0.399 | 0.110 | 0.153 |
| 9. 1 mg/kg AC002743 | 0.844 | 0.330 | 0.543 | 0.643 | 0.120 | 0.148 |
| 10. 1 mg/kg AC002744 | 0.315 | 0.031 | 0.034 | 0.196 | 0.072 | 0.113 |
| 11. 1 mg/kg AC002745 | 0.376 | 0.083 | 0.106 | 0.382 | 0.060 | 0.071 |

Groups 2-11 showed reduction of Adipoq in both tissues. In particular, group 10 showed ˜68% knockdown of Adiponectin in iWAT and ˜80% knockdown of Adiponectin in pgWAT.

### Example 13. In Vivo Administration of Lipid-Linked RNAi Agents in Mice

On study day 1, female C57bl/6 mice were injected with either saline or RNAi agent formulated in saline. Five (n=5) animals were dosed in each group at 250 μL/25 g body weight with saline or RNAi agent solution (at 0.75 mg/kg, 1.5 mg/kg, or 3 mg/kg). Animals were injected subcutaneously (SQ) to the dosing regimen of Table 23.

TABLE 23
Dosing regimen for mice of Example 13.

| Group | Dosing Material | Dosing Regimen |
|---|---|---|
| 1 | Saline | Single SQ Injection on Day 1 |
| 2 | 0.75 mg/kg AC002571 | Single SQ Injection on Day 1 |
| 3 | 1.5 mg/kg AC002571 | Single SQ Injection on Day 1 |
| 4 | 3 mg/kg AC002571 | Single SQ Injection on Day 1 |
| 5 | 0.75 mg/kg AC002577 | Single SQ Injection on Day 1 |
| 6 | 1.5 mg/kg AC002577 | Single SQ Injection on Day 1 |
| 7 | 3 mg/kg AC002577 | Single SQ Injection on Day 1 |
| 8 | 0.75 mg/kg AC002650 | Single SQ Injection on Day 1 |
| 9 | 1.5 mg/kg AC002650 | Single SQ Injection on Day 1 |
| 10 | 3 mg/kg AC002650 | Single SQ Injection on Day 1 |

On day 1 prior to dosing, serum was collected. On days 8 and 15 post dose, serum was collected. On day 15 post dosing, animals were sacrificed, adipose tissues were harvested, and the inguinal white adipose tissue (iWAT) and perigonadal white adipose tissue (pgWAT) were collected. Mouse adiponectin in serum was analyzed via enzyme-linked immunoassay (ELISA) assay (R&D Systems, catalog MRP300), normalized pre-dose and control group 1 (Saline). Expression of mouse Adipoq in each tissue was determined using qPCR, with mouse mArl1 as control. Average Adipoq expression for each animal in each tissue was normalized relative to pre-dose and control group 1 (Saline). Results are shown in Table 24A and 24B below.

TABLE 24A
Average relative expression of Adiponectin
in serum (ELISA) in mice of Example 13.

| | |
|---|---|
| Day 1 Pre-dose | Day 8 |

| | | Std Dev | | Std Dev |
|---|---|---|---|---|
| Group ID | Avg | (+/−) | Avg | (+/−) |
| 1. Saline | 1.000 | 0.000 | 1.000 | 0.195 |
| 2. 0.75 mg/kg AC002571 | 1.000 | 0.000 | 0.337 | 0.066 |
| 3. 1.5 mg/kg AC002571 | 1.000 | 0.000 | 0.150 | 0.026 |
| 4. 3 mg/kg AC002571 | 1.000 | 0.000 | 0.119 | 0.024 |
| 5. 0.75 mg/kg AC002577 | 1.000 | 0.000 | 0.327 | 0.045 |
| 6. 1.5 mg/kg AC002577 | 1.000 | 0.000 | 0.165 | 0.023 |
| 7. 3 mg/kg AC002577 | 1.000 | 0.000 | 0.079 | 0.013 |
| 8. 0.75 mg/kg AC002650 | 1.000 | 0.000 | 0.127 | 0.008 |
| 9. 1.5 mg/kg AC002650 | 1.000 | 0.000 | 0.140 | 0.035 |
| 10. 3 mg/kg AC002650 | 1.000 | 0.000 | 0.130 | 0.034 |

Day 15

| | | Std Dev |
|---|---|---|
| Group ID | Avg | (+/−) |
| 1. Saline | 1.000 | 0.182 |
| 2. 0.75 mg/kg AC002571 | 0.247 | 0.059 |
| 3. 1.5 mg/kg AC002571 | 0.098 | 0.034 |
| 4. 3 mg/kg AC002571 | 0.063 | 0.014 |
| 5.0.75 mg/kg AC002577 | 0.248 | 0.042 |
| 6. 1.5 mg/kg AC002577 | 0.095 | 0.010 |
| 7. 3 mg/kg AC002577 | 0.040 | 0.006 |
| 8. 0.75 mg/kg AC002650 | 0.082 | 0.010 |
| 9. 1.5 mg/kg AC002650 | 0.079 | 0.018 |
| 10. 3 mg/kg AC002650 | 0.064 | 0.014 |

Groups 2-10 showed reduction of Adiponectin in serum at both time points measured. In particular, group 8, animals dosed with an RNAi agent comprising a 5′-terminal CNR1 SM2-1-a and a 3′-terminal LP-378a moiety, showed greater than 90% knockdown of Adiponectin at day 15 at only a 0.75 mg/kg dose.

TABLE 24B
Average relative expression of Adipoq
(qPCR) in mice, Day 15, of Example 13.

| | mAdipoq (iWAT) | | | mAdipoq (pgWAT) | | |
|---|---|---|---|---|---|---|
| Group ID | Rel Exp | Low | High | Rel Exp | Low | High |
| 1. Saline | 1.000 | 0.158 | 0.187 | 1.000 | 0.432 | 0.760 |
| 2. 0.75 mg/kg AC002571 | 0.348 | 0.102 | 0.145 | 0.361 | 0.142 | 0.233 |
| 3. 1.5 mg/kg AC002571 | 0.246 | 0.046 | 0.057 | 0.241 | 0.059 | 0.078 |
| 4. 3 mg/kg AC002571 | 0.158 | 0.043 | 0.058 | 0.188 | 0.039 | 0.048 |
| 5. 0.75 mg/kg AC002577 | 0.567 | 0.129 | 0.167 | 0.419 | 0.065 | 0.077 |
| 6. 1.5 mg/kg AC002577 | 0.257 | 0.025 | 0.028 | 0.268 | 0.055 | 0.069 |
| 7. 3 mg/kg AC002577 | 0.169 | 0.022 | 0.025 | 0.127 | 0.027 | 0.035 |
| 8. 0.75 mg/kg AC002650 | 0.272 | 0.026 | 0.029 | 0.320 | 0.046 | 0.054 |
| 9. 1.5 mg/kg AC002650 | 0.161 | 0.027 | 0.033 | 0.195 | 0.043 | 0.056 |
| 10. 3 mg/kg AC002650 | 0.185 | 0.017 | 0.019 | 0.168 | 0.031 | 0.037 |

Groups 2-10 showed reduction of Adipoq in both tissues. In particular, group 8 showed ˜70% knockdown of Adiponectin in both iWAT and pgWAT at day 15, at only a 0.75 mg/kg dose.

### Example 14. In Vivo Administration of Lipid-Linked RNAi Agents in Mice

On study day 1, female C57bl/6 mice were injected with either saline or RNAi agent formulated in saline. Five (n=5) animals were dosed in each group at 250 μL/25 g body weight with saline or RNAi agent solution (at 1.5 mg/kg). Animals were injected subcutaneously (SQ) to the dosing regimen of Table 25.

TABLE 25
Dosing regimen for mice of Example 14.

| Group | Dosing Material | Dosing Regimen |
|---|---|---|
| 1 | Saline | Single SQ Injection on Day 1 |
| 2 | 1.5 mg/kg AC002571 | Single SQ Injection on Day 1 |
| 3 | 1.5 mg/kg AC002574 | Single SQ Injection on Day 1 |
| 4 | 1.5 mg/kg AC002649 | Single SQ Injection on Day 1 |
| 5 | 1.5 mg/kg AC002703 | Single SQ Injection on Day 1 |
| 6 | 1.5 mg/kg AC002704 | Single SQ Injection on Day 1 |
| 7 | 1.5 mg/kg AC002705 | Single SQ Injection on Day 1 |
| 8 | 1.5 mg/kg AC002706 | Single SQ Injection on Day 1 |
| 9 | 1.5 mg/kg AC002707 | Single SQ Injection on Day 1 |

On day 1 prior to dosing, serum was collected. On days 8 and 15 post dose, serum was collected. On day 15 post dosing, animals were sacrificed, adipose tissues were harvested, and the inguinal white adipose tissue (iWAT) and perigonadal white adipose tissue (pgWAT) were collected. Mouse adiponectin in serum was analyzed via enzyme-linked immunoassay (ELISA) assay (R&D Systems, catalog MRP300), normalized pre-dose and control group 1 (Saline). Expression of mouse Adipoq in each tissue was determined using qPCR, with mouse mAr1 as control. Average Adipoq expression for each animal in each tissue was normalized relative to pre-dose and control group 1 (Saline). Results are shown in Table 26A and 26B below.

TABLE 26A
Average relative expression of Adiponectin
in serum (ELISA) in mice of Example 14.

| | |
|---|---|
| Day 1 Pre-dose | Day 8 |

| | | Std Dev | | Std Dev |
|---|---|---|---|---|
| Group ID | Avg | (+/−) | Avg | (+/−) |
| 1. Saline | 1.000 | 0.000 | 1.000 | 0.190 |
| 2. 1.5 mg/kg AC002571 | 1.000 | 0.000 | 0.161 | 0.047 |
| 3. 1.5 mg/kg AC002574 | 1.000 | 0.000 | 0.138 | 0.028 |
| 4. 1.5 mg/kg AC002649 | 1.000 | 0.000 | 0.162 | 0.028 |
| 5. 1.5 mg/kg AC002703 | 1.000 | 0.000 | 0.218 | 0.125 |
| 6. 1.5 mg/kg AC002704 | 1.000 | 0.000 | 0.402 | 0.084 |
| 7. 1.5 mg/kg AC002705 | 1.000 | 0.000 | 0.331 | 0.068 |
| 8. 1.5 mg/kg AC002706 | 1.000 | 0.000 | 0.384 | 0.059 |
| 9. 1.5 mg/kg AC002707 | 1.000 | 0.000 | 0.360 | 0.055 |

Day 15

| | | Std Dev |
|---|---|---|
| Group ID | Avg | (+/−) |
| 1. Saline | 1.000 | 0.242 |
| 2. 1.5 mg/kg AC002571 | 0.077 | 0.019 |
| 3. 1.5 mg/kg AC002574 | 0.067 | 0.017 |
| 4. 1.5 mg/kg AC002649 | 0.094 | 0.015 |
| 5. 1.5 mg/kg AC002703 | 0.145 | 0.081 |
| 6. 1.5 mg/kg AC002704 | 0.307 | 0.072 |
| 7. 1.5 mg/kg AC002705 | 0.237 | 0.034 |
| 8. 1.5 mg/kg AC002706 | 0.295 | 0.052 |
| 9. 1.5 mg/kg AC002707 | 0.251 | 0.027 |

Groups 2-9 showed reduction of Adiponectin in serum at both time points. In particular, group 4, animals dosed with an RNAi agent comprising a 5′-terminal 379-a and a 3′-terminal LP-371 a moiety, showed greater than 90% knockdown of Adiponectin in serum at day 15.

TABLE 26B
Average relative expression of Adipoq
(qPCR) in mice, Day 15, of Example 14.

| | mAdipoq (iWAT) | | | mAdipoq (pgWAT) | | |
|---|---|---|---|---|---|---|
| Group ID | Rel Exp | Low | High | Rel Exp | Low | High |
| 1. Saline | 1.000 | 0.165 | 0.197 | 1.000 | 0.158 | 0.188 |
| 2. 1.5 mg/kg AC002571 | 0.200 | 0.056 | 0.078 | 0.089 | 0.027 | 0.039 |
| 3. 1.5 mg/kg AC002574 | 0.211 | 0.023 | 0.026 | 0.132 | 0.024 | 0.030 |
| 4. 1.5 mg/kg AC002649 | 0.233 | 0.054 | 0.071 | 0.169 | 0.041 | 0.055 |
| 5. 1.5 mg/kg AC002703 | 0.223 | 0.020 | 0.023 | 0.217 | 0.050 | 0.064 |
| 6. 1.5 mg/kg AC002704 | 0.499 | 0.113 | 0.147 | 0.434 | 0.067 | 0.079 |
| 7. 1.5 mg/kg AC002705 | 0.489 | 0.116 | 0.152 | 0.427 | 0.115 | 0.157 |
| 8. 1.5 mg/kg AC002706 | 0.516 | 0.163 | 0.239 | 0.427 | 0.073 | 0.089 |
| 9. 1.5 mg/kg AC002707 | 0.399 | 0.077 | 0.096 | 0.398 | 0.063 | 0.074 |

Groups 2-9 showed reduction of Adipoq in both tissues. In particular, group 4 showed greater than 75% of knockdown in both iWAT and pgWAT at day 15.

### Example 15. In Vivo Administration of Lipid-Linked RNAi Agents in Mice

On study day 1, female C57bl/6 mice were injected with either saline or RNAi agent formulated in saline. Five (n=5) animals were dosed in each group at 250 μL/25 g body weight with saline or RNAi agent solution (at 1.5 mg/kg). Animals were injected subcutaneously (SQ) to the dosing regimen of Table 27.

TABLE 27
Dosing regimen for mice of Example 15.

| Group | Dosing Material | Dosing Regimen |
|---|---|---|
| 1 | Saline | Single SQ Injection on Day 1 |
| 2 | 1.5 mg/kg AC002675 | Single SQ Injection on Day 1 |
| 3 | 1.5 mg/kg AC002571 | Single SQ Injection on Day 1 |
| 4 | 1.5 mg/kg AC002709 | Single SQ Injection on Day 1 |
| 5 | 1.5 mg/kg AC002577 | Single SQ Injection on Day 1 |
| 6 | 1.5 mg/kg AC002680 | Single SQ Injection on Day 1 |
| 7 | 1.5 mg/kg AC002681 | Single SQ Injection on Day 1 |

On day 1 prior to dosing, serum was collected. On days 8 and 15 post dose, serum was collected. On day 15 post dosing, animals were sacrificed, adipose tissues were harvested, and the inguinal white adipose tissue (iWAT) and perigonadal white adipose tissue (pgWAT) were collected. Mouse adiponectin in serum was analyzed via enzyme-linked immunoassay (ELISA) assay (R&D Systems, catalog MRP300), normalized pre-dose and control group 1 (Saline). Expression of mouse Adipoq in each tissue was determined using qPCR, with mouse mAr1 as control. Average Adipoq expression for each animal in each tissue was normalized relative to pre-dose and control group 1 (Saline). Results are shown in Table 28A and 28B below.

TABLE 28A
Average relative expression of Adiponectin
in serum (ELISA) in mice of Example 15.

| | |
|---|---|
| Day 1 Pre-dose | Day 8 |

| | | Std Dev | | Std Dev |
|---|---|---|---|---|
| Group ID | Avg | (+/−) | Avg | (+/−) |
| 1. Saline | 1.000 | 0.000 | 1.000 | 0.139 |
| 2. 1.5 mg/kg AC002675 | 1.000 | 0.000 | 0.254 | 0.153 |
| 3. 1.5 mg/kg AC002571 | 1.000 | 0.000 | 0.131 | 0.054 |
| 4. 1.5 mg/kg AC002709 | 1.000 | 0.000 | 0.273 | 0.084 |
| 5. 1.5 mg/kg AC002577 | 1.000 | 0.000 | 0.168 | 0.072 |
| 6. 1.5 mg/kg AC002680 | 1.000 | 0.000 | 0.356 | 0.095 |
| 7. 1.5 mg/kg AC002681 | 1.000 | 0.000 | 0.411 | 0.078 |

Day 15

| | | Std Dev |
|---|---|---|
| Group ID | Avg | (+/−) |
| 1. Saline | 1.000 | 0.145 |
| 2. 1.5 mg/kg AC002675 | 0.180 | 0.132 |
| 3. 1.5 mg/kg AC002571 | 0.088 | 0.056 |
| 4. 1.5 mg/kg AC002709 | 0.167 | 0.051 |
| 5. 1.5 mg/kg AC002577 | 0.095 | 0.046 |
| 6. 1.5 mg/kg AC002680 | 0.204 | 0.066 |
| 7. 1.5 mg/kg AC002681 | 0.257 | 0.071 |

Groups 2-7 showed reduction of Adiponectin in serum at both time points. In particular, group 5, animals dosed with an RNAi agent comprising a 5′-terminal 379-a and a 3′-terminal LP-378a moiety, showed greater than 90% knockdown of Adiponectin in serum at day 15.

TABLE 28B
Average relative expression of Adipoq
(qPCR) in mice, Day 15, of Example 15.

| | mAdipoq (iWAT) | | | mAdipoq (pgWAT) | | |
|---|---|---|---|---|---|---|
| Group ID | Rel Exp | Low | High | Rel Exp | Low | High |
| 1. Saline | 1.000 | 0.204 | 0.256 | 1.000 | 0.253 | 0.338 |
| 2. 1.5 mg/kg AC002675 | 0.383 | 0.133 | 0.203 | 0.398 | 0.121 | 0.175 |
| 3. 1.5 mg/kg AC002571 | 0.315 | 0.130 | 0.223 | 0.265 | 0.082 | 0.119 |
| 4. 1.5 mg/kg AC002709 | 0.427 | 0.154 | 0.242 | 0.338 | 0.102 | 0.146 |
| 5. 1.5 mg/kg AC002577 | 0.278 | 0.091 | 0.135 | 0.225 | 0.056 | 0.074 |
| 6. 1.5 mg/kg AC002680 | 0.652 | 0.177 | 0.243 | 0.508 | 0.139 | 0.191 |
| 7. 1.5 mg/kg AC002681 | 0.667 | 0.220 | 0.327 | 0.502 | 0.120 | 0.158 |

Groups 2-7 showed reduction of Adipoq in both tissues. In particular, group 5 showed greater than 70m knockdown in iWAT and pgWAT at day 15.

### Example 16. In Vivo Administration of Lipid-Linked RNAi Agents in Mice

On study day 1, female C57bl/6 mice were injected with either saline or RNAi agent formulated in saline. Five (n=5) animals were dosed in each group at 250 L/25 g body weight with saline or RNAi agent solution (at 2 mg/kg). Animals were injected subcutaneously (SQ) or intravenously (IV) via left posterior tibial vein (LPTV) to the dosing regimen of Table 29.

TABLE 29
Dosing regimen for mice of Example 16.

| Group | Dosing Material | Dosing Regimen |
|---|---|---|
| 1 | Saline | Single IV Injection on Day 1 |
| 2 | 2 mg/kg AC002571 | Single IV Injection on Day 1 |
| 3 | 2 mg/kg AC002648 | Single IV Injection on Day 1 |
| 4 | 2 mg/kg AC002649 | Single IV Injection on Day 1 |
| 5 | 2 mg/kg AC002577 | Single IV Injection on Day 1 |
| 6 | 2 mg/kg AC002650 | Single IV Injection on Day 1 |
| 7 | 2 mg/kg AC002571 | Single SQ Injection on Day 1 |
| 8 | 2 mg/kg AC002648 | Single SQ Injection on Day 1 |
| 9 | 2 mg/kg AC002649 | Single SQ Injection on Day 1 |
| 10 | 2 mg/kg AC002577 | Single SQ Injection on Day 1 |
| 11 | 2 mg/kg AC002650 | Single SQ Injection on Day 1 |
| 12 | 2 mg/kg AC002626 | Single SQ Injection on Day 1 |

On day 1 prior to dosing, serum was collected. On days 8 and 15 post dose, serum was collected. On day 15 post dosing, animals were sacrificed, adipose tissues were harvested, and the inguinal white adipose tissue (iWAT) and perigonadal white adipose tissue (pgWAT) were collected. Mouse adiponectin in serum was analyzed via enzyme-linked immunoassay (ELISA) assay (R&D Systems, catalog MRP300), normalized pre-dose and control group 1 (Saline). Expression of mouse Adipoq in each tissue was determined using qPCR, with mouse mArl1 as control. Average Adipoq expression for each animal in each tissue was normalized relative to pre-dose and control group 1 (Saline). Results are shown in Table 30A and 30B below.

TABLE 30A
Average relative expression of Adiponectin
in serum (ELISA) in mice of Example 16.

| | |
|---|---|
| Day 1 Pre-dose | Day 8 |

| | | Std Dev | | Std Dev |
|---|---|---|---|---|
| Group ID | Avg | (+/−) | Avg | (+/−) |
| 1. Saline | 1.000 | 0.000 | 1.000 | 0.020 |
| 2. 2 mg/kg AC002571 | 1.000 | 0.000 | 0.184 | 0.052 |
| 3. 2 mg/kg AC002648 | 1.000 | 0.000 | 0.632 | 0.110 |
| 4. 2 mg/kg AC002649 | 1.000 | 0.000 | 0.138 | 0.059 |
| 5. 2 mg/kg AC002577 | 1.000 | 0.000 | 0.205 | 0.088 |
| 6. 2 mg/kg AC002650 | 1.000 | 0.000 | 0.096 | 0.022 |
| 7. 2 mg/kg AC002571 | 1.000 | 0.000 | 0.182 | 0.033 |
| 8. 2 mg/kg AC002648 | 1.000 | 0.000 | 0.536 | 0.209 |
| 9. 2 mg/kg AC002649 | 1.000 | 0.000 | 0.123 | 0.008 |
| 10. 2 mg/kg AC002577 | 1.000 | 0.000 | 0.189 | 0.070 |
| 11. 2 mg/kg AC002650 | 1.000 | 0.000 | 0.163 | 0.062 |
| 12. 2 mg/kg AC002626 | 1.000 | 0.000 | 0.425 | 0.174 |

Day 15

| | | Std Dev |
|---|---|---|
| Group ID | Avg | (+/−) |
| 1. Saline | 1.000 | 0.077 |
| 2. 2 mg/kg AC002571 | 0.113 | 0.033 |
| 3. 2 mg/kg AC002648 | 0.442 | 0.083 |
| 4. 2 mg/kg AC002649 | 0.068 | 0.025 |
| 5. 2 mg/kg AC002577 | 0.114 | 0.029 |
| 6. 2 mg/kg AC002650 | 0.046 | 0.006 |
| 7. 2 mg/kg AC002571 | 0.092 | 0.047 |
| 8. 2 mg/kg AC002648 | 0.288 | 0.095 |
| 9. 2 mg/kg AC002649 | 0.051 | 0.006 |
| 10. 2 mg/kg AC002577 | 0.079 | 0.036 |
| 11. 2 mg/kg AC002650 | 0.051 | 0.015 |
| 12. 2 mg/kg AC002626 | 0.156 | 0.067 |

Groups 2-12 showed reduction of Adiponectin in serum at both time points. In particular, groups 4 (IV) and 9 (SQ), animals dosed with an RNAi agent comprising a 5′-terminal 379-a and a 3′-terminal LP-371a moiety, showed ˜95% knockdown of Adiponectin in serum at day 15.

TABLE 30B
Average relative expression of Adipoq
(qPCR) in mice, Day 15, of Example 16.

| | mAdipoq (iWAT) | | | mAdipoq (pgWAT) | | |
|---|---|---|---|---|---|---|
| Group ID | Rel Exp | Low | High | Rel Exp | Low | High |
| 1. Saline | 1.000 | 0.235 | 0.306 | 1.000 | 0.141 | 0.164 |
| 2. 2 mg/kg AC002571 | 0.208 | 0.056 | 0.076 | 0.147 | 0.044 | 0.063 |
| 3. 2 mg/kg AC002648 | 0.738 | 0.095 | 0.109 | 0.613 | 0.103 | 0.124 |
| 4. 2 mg/kg AC002649 | 0.153 | 0.057 | 0.091 | 0.118 | 0.031 | 0.043 |
| 5. 2 mg/kg AC002577 | 0.226 | 0.073 | 0.108 | 0.146 | 0.043 | 0.061 |
| 6. 2 mg/kg AC002650 | 0.202 | 0.018 | 0.020 | 0.132 | 0.013 | 0.014 |
| 7. 2 mg/kg AC002571 | 0.258 | 0.051 | 0.064 | 0.144 | 0.028 | 0.034 |
| 8. 2 mg/kg AC002648 | 0.767 | 0.103 | 0.120 | 0.436 | 0.039 | 0.043 |
| 9. 2 mg/kg AC002649 | 0.210 | 0.041 | 0.051 | 0.100 | 0.025 | 0.034 |
| 10. 2 mg/kg AC002577 | 0.283 | 0.085 | 0.121 | 0.205 | 0.046 | 0.060 |
| 11. 2 mg/kg AC002650 | 0.150 | 0.028 | 0.035 | 0.102 | 0.033 | 0.048 |
| 12. 2 mg/kg AC002626 | 0.377 | 0.078 | 0.098 | 0.378 | 0.120 | 0.176 |

Groups 2-12 showed reduction of Adipoq in both tissues. In particular, groups 4 and 9 showed impressive knockdown in both tissues.

### Example 17. In Vivo Administration of Lipid-Linked RNAi Agents in Mice

On study day 1, female C57bl/6 mice were injected with either saline or RNAi agent formulated in saline. Five (n=5) animals were dosed in each group at 250 μL/25 g body weight with saline or RNAi agent solution (at 0.75 mg/kg, 1.5 mg/kg, or 3 mg/kg). Animals were injected intravenously (IV) via left posterior tibial vein (LPTV) to the dosing regimen of Table 31.

TABLE 31
Dosing regimen for mice of Example 17.

| Group | Dosing Material | Dosing Regimen |
|---|---|---|
| 1 | Saline | Single IV Injection on Day 1 |
| 2 | 0.75 mg/kg AC002571 | Single IV Injection on Day 1 |
| 3 | 1.5 mg/kg AC002571 | Single IV Injection on Day 1 |
| 4 | 3 mg/kg AC002571 | Single IV Injection on Day 1 |
| 5 | 0.75 mg/kg AC002577 | Single IV Injection on Day 1 |
| 6 | 1.5 mg/kg AC002577 | Single IV Injection on Day 1 |
| 7 | 3 mg/kg AC002577 | Single IV Injection on Day 1 |
| 8 | 0.75 mg/kg AC002650 | Single IV Injection on Day 1 |
| 9 | 1.5 mg/kg AC002650 | Single IV Injection on Day 1 |
| 10 | 3 mg/kg AC002650 | Single IV Injection on Day 1 |

On day 1 prior to dosing, serum was collected. On days 8 and 15 post dose, serum was collected. On day 15 post dosing, animals were sacrificed, adipose tissues were harvested, and the inguinal white adipose tissue (iWAT) and perigonadal white adipose tissue (pgWAT) were collected. Mouse adiponectin in serum was analyzed via enzyme-linked immunoassay (ELISA) assay (R&D Systems, catalog MRP300), normalized pre-dose and control group 1 (Saline). Expression of mouse Adipoq in each tissue was determined using qPCR, with mouse mArl1 as control. Average Adipoq expression for each animal in each tissue was normalized relative to pre-dose and control group 1 (Saline). Results are shown in Table 32A and 32B below.

TABLE 32A
Average relative expression of Adiponectin
in serum (ELISA) in mice of Example 17.

| | |
|---|---|
| Day 1 Pre-dose | Day 8 |

| | | Std Dev | | Std Dev |
|---|---|---|---|---|
| Group ID | Avg | (+/−) | Avg | (+/−) |
| 1. Saline | 1.000 | 0.000 | 1.000 | 0.138 |
| 2. 0.75 mg/kg AC002571 | 1.000 | 0.000 | 0.334 | 0.062 |
| 3. 1.5 mg/kg AC002571 | 1.000 | 0.000 | 0.169 | 0.079 |
| 4. 3 mg/kg AC002571 | 1.000 | 0.000 | 0.093 | 0.034 |
| 5. 0.75 mg/kg AC002577 | 1.000 | 0.000 | 0.254 | 0.105 |
| 6. 1.5 mg/kg AC002577 | 1.000 | 0.000 | 0.167 | 0.072 |
| 7. 3 mg/kg AC002577 | 1.000 | 0.000 | 0.090 | 0.026 |
| 8. 0.75 mg/kg AC002650 | 1.000 | 0.000 | 0.158 | 0.043 |
| 9. 1.5 mg/kg AC002650 | 1.000 | 0.000 | 0.082 | 0.028 |
| 10. 3 mg/kg AC002650 | 1.000 | 0.000 | 0.053 | 0.007 |

Day 15

| | | Std Dev |
|---|---|---|
| Group ID | Avg | (+/−) |
| 1. Saline | 1.000 | 0.224 |
| 2. 0.75 mg/kg AC002571 | 0.251 | 0.031 |
| 3. 1.5 mg/kg AC002571 | 0.120 | 0.065 |
| 4. 3 mg/kg AC002571 | 0.098 | 0.039 |
| 5. 0.75 mg/kg AC002577 | 0.164 | 0.054 |
| 6. 1.5 mg/kg AC002577 | 0.104 | 0.062 |
| 7. 3 mg/kg AC002577 | 0.074 | 0.030 |
| 8. 0.75 mg/kg AC002650 | 0.075 | 0.019 |
| 9. 1.5 mg/kg AC002650 | 0.037 | 0.014 |
| 10. 3 mg/kg AC002650 | 0.040 | 0.007 |

Groups 2-10 showed reduction of Adiponectin in serum at both time points. In particular, group 8, animals dosed with an RNAi agent comprising a 5′-terminal CNR1 SM2-1-a and a 3′-terminal LP-378-a moiety, showed greater than 90% knockdown of Adiponectin in serum at day 15 at only a 0.75 mg/kg dose.

TABLE 32B
Average relative expression of Adipoq
(qPCR) in mice, Day 15, of Example 17.

| | mAdipoq (iWAT) | | | mAdipoq (pgWAT) | | |
|---|---|---|---|---|---|---|
| Group ID | Rel Exp | Low | High | Rel Exp | Low | High |
| 1. Saline | 1.000 | 0.148 | 0.174 | 1.000 | 0.215 | 0.273 |
| 2. 0.75 mg/kg AC002571 | 0.584 | 0.104 | 0.126 | 0.453 | 0.066 | 0.077 |
| 3. 1.5 mg/kg AC002571 | 0.456 | 0.107 | 0.140 | 0.289 | 0.070 | 0.092 |
| 4. 3 mg/kg AC002571 | 0.248 | 0.071 | 0.100 | 0.160 | 0.036 | 0.047 |
| 5. 0.75 mg/kg AC002577 | 0.455 | 0.118 | 0.160 | 0.349 | 0.068 | 0.085 |
| 6. 1.5 mg/kg AC002577 | 0.430 | 0.098 | 0.127 | 0.295 | 0.068 | 0.089 |
| 7. 3 mg/kg AC002577 | 0.185 | 0.020 | 0.023 | 0.140 | 0.028 | 0.034 |
| 8.0.75 mg/kg AC002650 | 0.306 | 0.050 | 0.060 | 0.227 | 0.043 | 0.053 |
| 9. 1.5 mg/kg AC002650 | 0.233 | 0.041 | 0.050 | 0.131 | 0.015 | 0.016 |
| 10. 3 mg/kg AC002650 | 0.111 | 0.010 | 0.011 | 0.075 | 0.025 | 0.038 |

Groups 2-10 showed reduction of Adipoq in both tissues. In particular, group 8 shows ˜70% knockdown of adiponectin in both iWAT and pgWAT.

### Example 18. In Vivo Administration of ALK7 RNAi Agents in Mice

ALK7 RNAi agents were evaluated in vivo in Mice using oligonucleotides comprising the PK/PD modulators LP-371-a and LP-379-a. On Day 1, five (n=5) female C57bl/6 Mice test animals were given a single subcutaneous (SQ) injection of 250 μl per 25 g body weight containing either 1.0 mg/kg (mpk), 3.0 mg/kg (mpk) of an ALK7 RNAI agent, or saline. Dosing was in accordance with Table 33 below.

TABLE 33
Dosing groups for the mice of Example 18.

| | | # of |
|---|---|---|
| Group ID | Dose (RNAi Agent) | Animals |
| 1. Saline | Day 1: Single SQ Injection | n = 5 |
| 2. 1.0 mg/kg AC004391 | Day 1: Single SQ Injection | n = 5 |
| 3. 3.0 mg/kg AC004391 | Day 1: Single SQ Injection | n = 5 |
| 4. 1.0 mg/kg AC004390 | Day 1: Single SQ Injection | n = 5 |
| 5. 3.0 mg/kg AC004390 | Day 1: Single SQ Injection | n = 5 |
| 6. 1.0 mg/kg AC004392 | Day 1: Single SQ Injection | n = 5 |
| 7. 3.0 mg/kg AC004392 | Day 1: Single SQ Injection | n = 5 |
| 8. 1.0 mg/kg AC005181 | Day 1: Single SQ Injection | n = 5 |
| 9. 3.0 mg/kg AC005181 | Day 1: Single SQ Injection | n = 5 |
| 10. 1.0 mg/kg AC005824 | Day 1: Single SQ Injection | n = 5 |
| 11. 3.0 mg/kg AC005824 | Day 1: Single SQ Injection | n = 5 |
| 12. 1.0 mg/kg AC005823 | Day 1: Single SQ Injection | n = 5 |
| 13. 3.0 mg/kg AC005823 | Day 1: Single SQ Injection | n = 5 |

ALK7 RNAi agents AC004391, AC004390, AC004392, and AC005181 target and initiate RNAi and RNA-induced silencing complex (RISC) of mouse ALK7. ALK7 RNAi agents AC005824 and AC005823 target and initiate RNAi and RNA-induced silencing complex (RISC) of human ALK7.

Five (n=5) mice were dosed in each group. Mice were injected subcutaneously (SQ) on day 1. On day 15, mice were euthanized, and ˜50 mg adipose tissues (inguinal white adipose tissue iWAT, perigonadal white adipose tissue pgWAT) were collected for analysis. Samples were analyzed by qPCR for mALK7 mRNA knockdown, using mARL1 as an endogenous control reference gene, and normalized to group 1 (saline). Average results for each group are shown in Table 34 below.

TABLE 34
Relative expression of ALK7 mRNA in various tissues analyzed
by qPCR for each of the dosing groups of Example 18.

| | iWAT | | | pgWAT | | |
|---|---|---|---|---|---|---|
| | Group Average (n = 5) | | | Group Average (n = 5) | | |
| | Rel. | Error | Error | Rel. | Error | Error |
| Group ID | Exp. | (Low) | (High) | Exp. | (Low) | (High) |
| 1. Saline | 1.000 | 0.111 | 0.124 | 1.000 | 0.144 | 0.168 |
| 2. 1.0 mg/kg AC004391 | 0.501 | 0.101 | 0.127 | 0.566 | 0.135 | 0.178 |
| 3. 3.0 mg/kg AC004391 | 0.504 | 0.085 | 0.103 | 0.503 | 0.097 | 0.121 |
| 4. 1.0 mg/kg AC004390 | 0.454 | 0.066 | 0.078 | 0.496 | 0.062 | 0.071 |
| 5. 3.0 mg/kg AC004390 | 0.339 | 0.097 | 0.137 | 0.394 | 0.142 | 0.223 |
| 6. 1.0 mg/kg AC004392 | 0.542 | 0.109 | 0.137 | 0.520 | 0.124 | 0.163 |
| 7. 3.0 mg/kg AC004392 | 0.376 | 0.095 | 0.127 | 0.542 | 0.125 | 0.163 |
| 8. 1.0 mg/kg AC005181 | 0.399 | 0.057 | 0.067 | 0.349 | 0.091 | 0.124 |
| 9. 3.0 mg/kg AC005181 | 0.337 | 0.069 | 0.087 | 0.187 | 0.063 | 0.096 |
| 10. 1.0 mg/kg AC005824 | 0.505 | 0.130 | 0.175 | 0.461 | 0.103 | 0.133 |
| 11. 3.0 mg/kg AC005824 | 0.294 | 0.039 | 0.044 | 0.347 | 0.062 | 0.075 |
| 12. 1.0 mg/kg AC005823 | 0.422 | 0.122 | 0.171 | 0.407 | 0.114 | 0.158 |
| 13. 3.0 mg/kg AC005823 | 0.345 | 0.100 | 0.141 | 0.322 | 0.104 | 0.154 |

As shown in Table 34, Groups 2-13 showed reductions in ALK7 in comparison to Group 1 dosed with no ALK7 RNAi agent, in both iWAT and pgWAT. More specifically, ALK7 RNAi agent AC005824 at 3.0 mg/kg dose achieved ˜70% ALK7 inhibition (0.294) in iWAT on Day 15; ALK7 RNAi agent AC005181 at 3.0 mg/kg dose achieved ˜81% ALK7 inhibition (0.187) in pgWAT on Day 15. Dose response was observed in Groups 4&5, 6&7, 8&9, 10&11, and 12&13 in iWAT, and also observed in Groups 2&3, 4&5, 8&9, 10&11, and 12&13 in pgWAT.

### Example 19. In Vivo Administration of ALK7 RNAi Agents in Cynomolgus Monkeys

ALK7 RNAi agents comprising the PK/PD modulators LP-371-a and LP-379-a are currently being tested in Cynomolgus monkeys for inhibition of ALK7. On Day 1 and Day 29, three (n=3) male Cynomolgus monkey test animals for each test group will be dosed with ALK7 RNAi agents formulated in saline (at 3.0 mg/kg), via subcutaneous (SQ) injection with syringe and needle in the mid-scapular region, at 0.3 mL/kg dose volume. Adipose biopsies will be collected from all test animals on Day −7 (pre-dose), 15, 29, 57, and 85. The dosing regimen will be in accordance with Table 35 below.

TABLE 35
Dosing for test animals of Example 19.

| | Dose | # of | | Dose |
|---|---|---|---|---|
| Group | (RNAi Agent) | Animals | Dosing Route | Volume |
| 1 | 3.0 mg/kg AC006188 | n = 3 | Day 1 & 29 SQ Injection | 0.3 mL/kg |
| 2 | 3.0 mg/kg AC006189 | n = 3 | Day 1 & 29 SQ Injection | 0.3 mL/kg |

All animals will be fasted for at least 12 less than 24 hours for scheduled blood collections and biopsy procedures. Blood collection site will be femoral vein. A saphenous vein (not used for dose administration) may be used as an alternative collection site.

Adipose biopsies will be collected as a sedated procedure. Sedation will be accomplished using Ketamine HCl (10 mg/kg) or Telazol (5-8 mg/kg), administered as an intramuscular (IM) injection and supplemented with Ketamine (5 mg/kg) as needed.

An approximate 3-5 cm skin incision will be made followed by collection of adipose tissue (optimally 50-150 mg). The skin will then be closed in a routine manner using suture (or alternate) materials maintaining aseptic technique.

Each biopsy site will be separated by at least 1-2 cm. Biopsies will be separated into 2 pieces (one piece of ˜25 to 75 mg and second piece of ˜25-75 mg) for each collection time point. Analgesics may be administered at veterinarian's discretion.

Blood will be collected into tubes containing no anticoagulant (serum separator tubes). Blood will be allowed to clot at ambient temperature prior to centrifugation to obtain serum.

Blood will be collected on Day -7, Day 1, Day 15, Day 29, Day 57, and Day 85, prior to liver biopsy sample collections or dose administration (when applicable), and from any animals found in moribund condition or sacrificed at an unscheduled interval.

Individual doses of ALK7 RNAi agents will be calculated based on the body weights recorded on each day of dosing.

The adipose biopsies and serum collected from the test animals will be used for analysis for ALK7 expression and additional biological parameters. Serum ALK7 mRNA expression levels will be quantified via qPCR, using cARL1 as an endogenous control reference gene, and normalized to Day −7.

This study is currently ongoing, and currently available data is shown below in Table 36.

TABLE 36
Serum ALK7 expression of Cynomolgus animals of Example 19.

| | Day −7 | | | Day 15 | | | Day 29 | | |
|---|---|---|---|---|---|---|---|---|---|
| | Rel. | Error | Error | Rel. | Error | Error | Rel. | Error | Error |
| Group ID | Exp. | Low | High | Exp. | Low | High | Exp. | Low | High |
| 1. 3.0 mg/kg AC006188 | 1.000 | 0.240 | 0.316 | 0.185 | 0.066 | 0.101 | 0.103 | 0.041 | 0.069 |
| 2. 3.0 mg/kg AC006189 | 1.000 | 0.086 | 0.094 | 0.143 | 0.077 | 0.166 | 0.138 | 0.032 | 0.042 |

Both Groups 1 and 2, AC006188 and AC006189 showed inhibition of ALK7 in cynomolgus monkeys.

### Example 20. In vivo administration of lipid-conjugated RNAi agents in mice

On study day 1, female C57bl/6 mice were injected with either saline or a lipid-conjugated RNAi agent formulated in saline. Five (n=5) animals were dosed in each group at 250 μL/25 g body weight with saline or RNAi agent solution (at 0.5 mg/kg or 1 mg/kg). Animals were injected subcutaneously (SQ) to the dosing regimen of Table 37.

TABLE 37
Dosing regimen for mice of Example 20.

| Group | Dosing Material | Dosing Regimen |
|---|---|---|
| 1 | Saline | Single SQ Injection on Day 1 |
| 2 | 0.5 mg/kg AC002772 | Single SQ Injection on Day 1 |
| 3 | 1 mg/kg AC002772 | Single SQ Injection on Day 1 |
| 4 | 0.5 mg/kg AC002649 | Single SQ Injection on Day 1 |
| 5 | 1 mg/kg AC002649 | Single SQ Injection on Day 1 |
| 6 | 0.5 mg/kg AC002571 | Single SQ Injection on Day 1 |
| 7 | 1 mg/kg AC002571 | Single SQ Injection on Day 1 |
| 8 | 0.5 mg/kg AC003478 | Single SQ Injection on Day 1 |
| 9 | 1 mg/kg AC003478 | Single SQ Injection on Day 1 |

The lipid-conjugated RNAi agents were designed such that the antisense included a nucleotide sequence complementary to the Adipoq gene transcript, which expresses the protein hormone adiponectin primarily in adipose tissue. Thus, the lipid-conjugated RNAi agents were designed to inhibit the expression of the Adipoq gene. On day 1 prior to dosing, serum was collected. On days 8, 15, and 22 post dose, serum was collected. On day 22 post dosing, animals were sacrificed, adipose tissues were harvested, and the inguinal white adipose tissue (iWAT) and perigonadal white adipose tissue (pgWAT) were collected. Mouse adiponectin in serum was analyzed via enzyme-linked immunoassay (ELISA) assay (R&D Systems, catalog MRP300), normalized pre-dose and control group 1 (Saline). Expression of mouse Adipoq in each tissue was determined using qPCR, with mouse mAr1 as control. Average Adipoq expression for each animal in each tissue was normalized relative to pre-dose and control group 1 (Saline). Results are shown in Table 6A and 6B below.

TABLE 37A
Average relative expression of Adiponectin
in serum (ELISA) in mice of Example 20.

| | |
|---|---|
| Day 1 Pre-dose | Day 8 |

| | | Std Dev | | Std Dev |
|---|---|---|---|---|
| Group ID | Avg | (+/−) | Avg | (+/−) |
| 1. Saline | 1.000 | 0.000 | 1.000 | 0.132 |
| 2. 0.5 mg/kg AC002772 | 1.000 | 0.000 | 1.132 | 0.092 |
| 3. 1 mg/kg AC002772 | 1.000 | 0.000 | 1.035 | 0.128 |
| 4. 0.5 mg/kg AC002649 | 1.000 | 0.000 | 0.327 | 0.059 |
| 5. 1 mg/kg AC002649 | 1.000 | 0.000 | 0.211 | 0.032 |
| 6. 0.5 mg/kg AC002571 | 1.000 | 0.000 | 0.393 | 0.080 |
| 7. 1 mg/kg AC002571 | 1.000 | 0.000 | 0.278 | 0.057 |
| 8. 0.5 mg/kg AC003478 | 1.000 | 0.000 | 0.651 | 0.195 |
| 9. 1 mg/kg AC003478 | 1.000 | 0.000 | 1.050 | 0.062 |

| | |
|---|---|
| Day 15 | Day 22 |

| | | Std Dev | | Std Dev |
|---|---|---|---|---|
| Group ID | Avg | (+/−) | Avg | (+/−) |
| 1. Saline | 1.000 | 0.211 | 1.000 | 0.209 |
| 2. 0.5 mg/kg AC002772 | 0.990 | 0.196 | 0.997 | 0.160 |
| 3. 1 mg/kg AC002772 | 0.953 | 0.201 | 1.021 | 0.293 |
| 4. 0.5 mg/kg AC002649 | 0.176 | 0.017 | 0.210 | 0.024 |
| 5. 1 mg/kg AC002649 | 0.116 | 0.019 | 0.107 | 0.024 |
| 6. 0.5 mg/kg AC002571 | 0.291 | 0.074 | 0.285 | 0.098 |
| 7. 1 mg/kg AC002571 | 0.133 | 0.022 | 0.210 | 0.044 |
| 8. 0.5 mg/kg AC003478 | 0.411 | 0.117 | 0.462 | 0.109 |
| 9. 1 mg/kg AC003478 | 0.211 | 0.075 | 0.206 | 0.081 |

Groups 4-9 showed reduction of Adiponectin in serum at all measured time points. Groups 2 and 3, which had no lipid moiety conjugates, showed poor knockdown. In particular, groups 4 and 5, animals dosed with an RNAi agent comprising a 5′-terminal LP-379-a and 3′-terminal LP-371-a moiety, showed impressive Adiponectin reduction relative to dose levels in serum at days 15 and 22.

TABLE 37B
Average relative expression of Adipoq
(qPCR) in mice, Day 22, of Example 4.

| | mAdipoq (iWAT) | | | mAdipoq (pgWAT) | | |
|---|---|---|---|---|---|---|
| Group ID | Rel Exp | Low | High | Rel Exp | Low | High |
| 1. Saline | 1.000 | 0.342 | 0.520 | 1.000 | 0.074 | 0.080 |
| 2. 0.5 mg/kg AC002772 | 0.722 | 0.122 | 0.146 | 0.579 | 0.122 | 0.155 |
| 3. 1 mg/kg AC002772 | 1.133 | 0.118 | 0.131 | 0.910 | 0.072 | 0.078 |
| 4. 0.5 mg/kg AC002649 | 0.211 | 0.070 | 0.105 | 0.206 | 0.063 | 0.092 |
| 5. 1 mg/kg AC002649 | 0.217 | 0.062 | 0.087 | 0.107 | 0.033 | 0.049 |
| 6. 0.5 mg/kg AC002571 | 0.709 | 0.061 | 0.067 | 0.467 | 0.060 | 0.069 |
| 7. 1 mg/kg AC002571 | 0.464 | 0.072 | 0.086 | 0.279 | 0.057 | 0.071 |
| 8. 0.5 mg/kg AC003478 | 0.719 | 0.079 | 0.088 | 0.381 | 0.068 | 0.082 |
| 9. 1 mg/kg AC003478 | 0.408 | 0.107 | 0.144 | 0.186 | 0.037 | 0.047 |

In particular, groups 4 and 5 showed impressive adiponectin reduction in iWAT and pgWAT at day 22.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A compound of Formula (I): 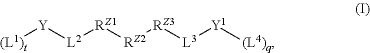
or a pharmaceutically acceptable salt thereof, wherein:
R^{Z1} and R^{Z3} each independently a bond or a capping residue;
R^{Z2} comprises an oligonucleotide containing about 8 to about 50 nucleotides, each of which may be independently modified or unmodified;
Y is a bond or a linker connecting at least one L¹ to L² when present, or to R^{Z1};
Y¹ is a bond or a linker connecting at least one L³ to L⁴ when present, or to R^{Z3};
L² and L³ are each independently a linker;
q is 1, 2, or 3, as valency permits;
t is 1, 2, or 3, as valency permits; and
L¹ and L⁴ are each independently a lipid comprising from about 10 to about 50 carbon atoms.

2. The compound or pharmaceutically acceptable salt of claim 1, wherein R^{Z1} is conjugated to a 5′ carbon atom of a modified or unmodified ribose molecule of R^{Z2}.

3. The compound or pharmaceutically acceptable salt of claim 1 or 2, wherein Y is a bond.

4. The compound or pharmaceutically acceptable salt of any one of claims 1-3, wherein Y is a linker connecting at least one L¹ to L² if present, or L¹ to R^{Z1}.

5. The compound or pharmaceutically acceptable salt of claims 1-4, wherein Y is a bivalent moiety connecting at least one L¹ to L² if present, or connecting L¹ to R^{Z1}.

6. The compound or pharmaceutically acceptable salt of any one of claims 1-5, wherein Y is of the formula: 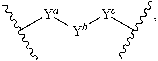
wherein:
Y^{a} and Y^{c} are each independently absent, —N(H)—, or —C(O)—;
Y^{b} is absent or substituted or unsubstituted heteroalkylene, substituted or unsubstituted carbocyclene, substituted or unsubstituted heterocyclene; substituted or unsubstituted arylene, or substituted or unsubstituted heteroarylene.

7. The compound or pharmaceutically acceptable salt of claim 6, wherein Y^{a} is —C(O)—.

8. The compound or pharmaceutically acceptable salt of claim 6, wherein Y^{a} is —N(H)—.

9. The compound or pharmaceutically acceptable of any one of claims 6-8, wherein Y^{c} is —(CO)—.

10. The compound or pharmaceutically acceptable salt of any one of claims 6-8, wherein Y^{c} is —N(H)—.

11. The compound or pharmaceutically acceptable salt of any one of claims 6-10, wherein Y^{b} is substituted or unsubstituted C₁-C₆ heteroalkylene.

12. The compound or pharmaceutically acceptable salt of any one of claims 6-11, wherein Y^{b} is of the formula

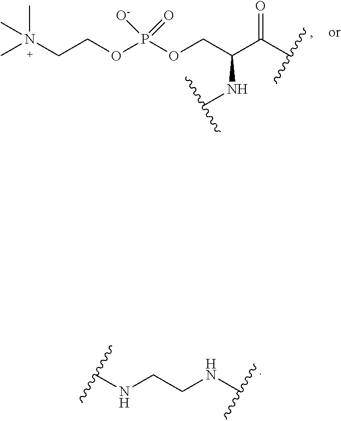

13. The compound or pharmaceutically acceptable salt of any one of claims 6-10, wherein Y^{b} substituted or unsubstituted carbocyclene.

14. The compound or pharmaceutically acceptable salt of any one of claims 6-10, or 13, wherein Y^{b} is of the formula

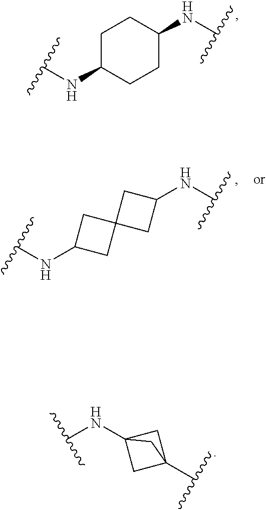

15. The compound or pharmaceutically acceptable salt of any one of claims 6-10, wherein Y^{b} substituted or unsubstituted heterocyclene.

16. The compound or pharmaceutically acceptable salt of any one of claims 6-10, or 15, wherein Y^{b} is of the formula

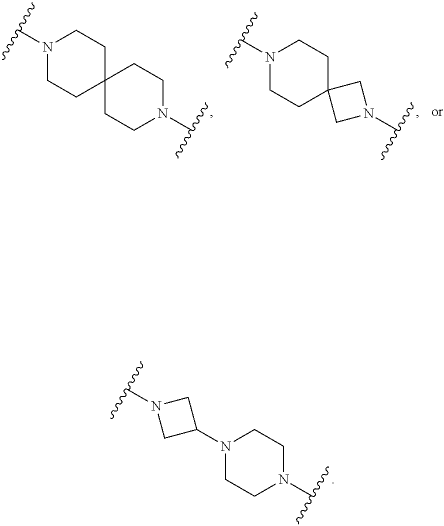 [SMILES: *N1CCC2(CC1)CCN(*)CC2 ; *N1CCC2(CC1)CN(*)C2 ; *N1CCN(C2CN(*)C2)CC1]

17. The compound or pharmaceutically acceptable salt of any one of claims 6-10, wherein Y^{b} substituted or unsubstituted arylene.

18. The compound or pharmaceutically acceptable salt of any one of claims 6-10, or 17, wherein Y^{b} substituted or unsubstituted phenylene.

19. The compound or pharmaceutically acceptable salt of any one of claims 6-10, or 17-18, wherein Y^{b} is of the formula

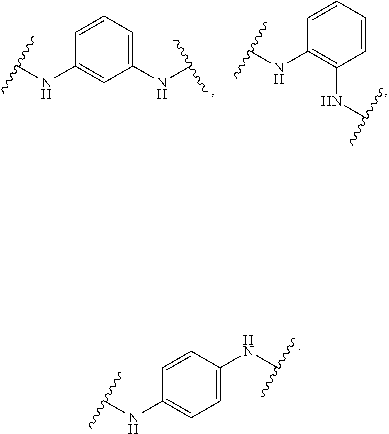 [SMILES: *Nc1cccc(N*)c1 ; *Nc1ccccc1N* ; *Nc1ccc(N*)cc1]

20. The compound or pharmaceutically acceptable salt of any one of claims 1-19, wherein Y is selected from the group consisting of —N(H)—C(O)—, —C(O)—N(H)—,

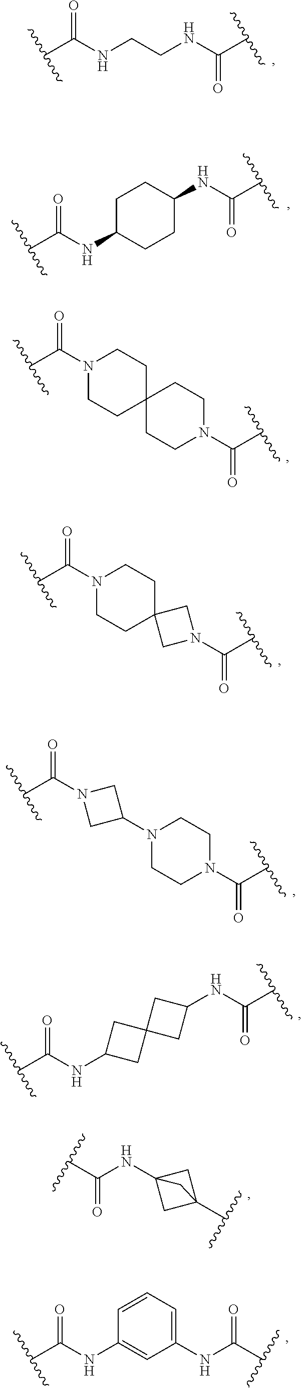

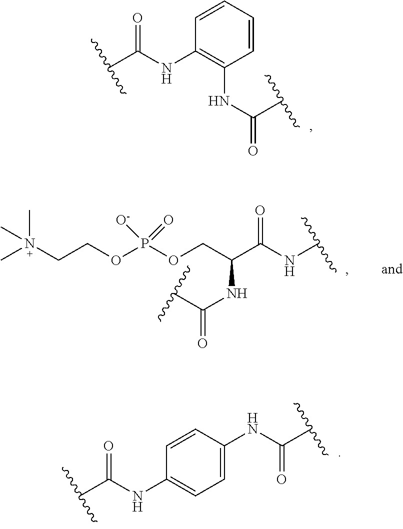

21. The compound or pharmaceutically acceptable salt of claim 1 or 2, wherein Y is a trivalent moiety connecting two L¹ groups to L², or connecting two L¹ groups to R^{Z1}.

22. The compound or pharmaceutically acceptable salt of claim 21, wherein Y is of the formula:

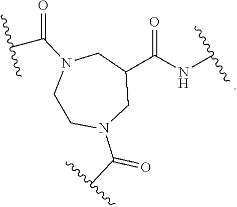 [SMILES: *NC(=O)C1CN(C(*)=O)CCN(C(*)=O)C1]

23. The compound or pharmaceutically acceptable salt of any one of claims 1-22, wherein L² is of the formula 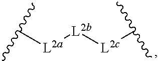
wherein:
L^{2a} is a bond or of the formula: 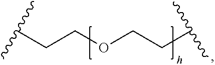
wherein h is an integer between 1 and 12;
L^{2b} is a bond or a chemical moiety formed by reacting a first reactive moiety with a second reactive moiety; and
L^{2c} is a bond or a bidentate linking group.

24. The compound or pharmaceutically acceptable salt of claim 23, wherein L^{2b} is a bond, —C(O)—, or
of the formula:

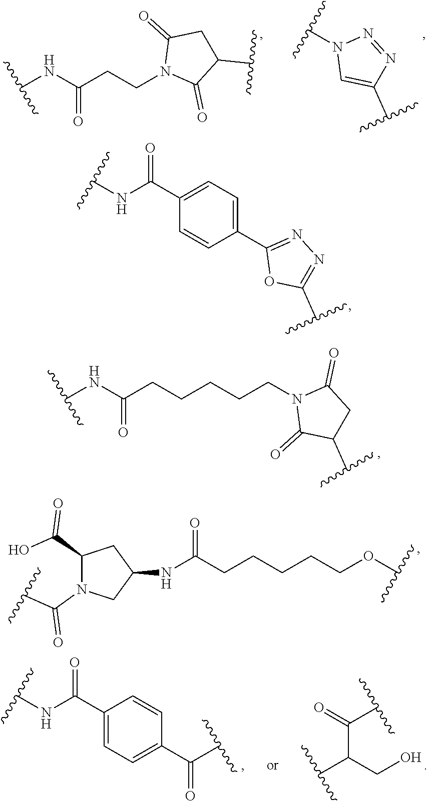

25. The compound or pharmaceutically acceptable salt of claim 23 or 24, wherein L^{2c} is

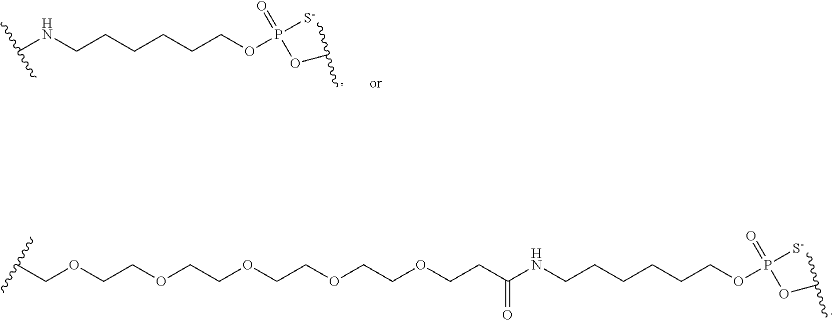 [SMILES: *NCCCCCCOP(=O)([S-])O* ; *COCCOCCOCCOCCOCCC(=O)NCCCCCCOP(=O)([S-])O*]

26. The compound or pharmaceutically acceptable salt of any one of claims 23-25, wherein h is 2, 3, 5, 9, or 10.

27. The compound or pharmaceutically acceptable salt of any one of claims 1-26, wherein at least one instance of L¹ is independently a straight chain lipid.

28. The compound or pharmaceutically acceptable salt of any one of claims 1-27, wherein at least one instance of L¹ is independently a saturated lipid.

29. The compound or pharmaceutically acceptable salt of any one of claims 1-28, wherein at least one instance of L¹ is independently of the formula: 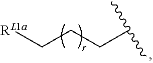
wherein
R^{L1a} is H, or CO₂H; and
r is an integer between 5 and 35.

30. The compound or pharmaceutically acceptable salt of claim 29, wherein R^{L1a} is H.

31. The compound or pharmaceutically acceptable salt of claim 29 or 30, wherein R^{L1a} is CO₂H.

32. The compound or pharmaceutically acceptable salt of any one of claims 29-31, wherein r is 8, 9, 10, 11, 12, 13, 14, 15, 16, 17, 18, 19, or 20.

33. The compound or pharmaceutically acceptable salt of any one of claims 29-32, wherein r is 9, 11, 12, 13, 14, 15, or 17.

34. The compound or pharmaceutically acceptable salt of claim 29, wherein r is 13.

35. The compound or pharmaceutically acceptable salt of claim 34, wherein R^{L1a} is H.

36. The compound or pharmaceutically acceptable salt of claim 29, wherein r is 12.

37. The compound or pharmaceutically acceptable salt of claim 36, wherein R^{L1a} is CO₂H.

38. The compound or pharmaceutically acceptable salt of any one of claims 1-37, wherein at least one instance of L¹ is independently a branched lipid.

39. The compound or pharmaceutically acceptable salt of any one of claims 1-38, wherein at least one instance of L¹ is independently of the formula 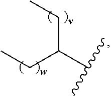
wherein:
w is an integer between 2 and 25; and
v is an integer between 2 and 25.

40. The compound or pharmaceutically acceptable salt of any one of claims 1-39, wherein at least one instance of L¹ is independently of the formula:

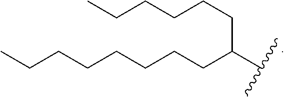 [SMILES: *C(CCCCCC)CCCCCCCC]

41. The compound or pharmaceutically acceptable salt of any one of claims 1-40, wherein at least one instance of L¹ is independently an unsaturated lipid.

42. The compound or pharmaceutically acceptable salt of any one of claims 1-41, wherein at least one instance of L¹ is independently a straight chain lipid comprising 1-8 alkenylene moieties.

43. The compound or pharmaceutically acceptable salt of any one of claims 1-42, wherein at least one instance of L¹ is independently of the formula: 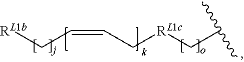
wherein:
R^{L1b} is —CH₃, or —CO₂H;
R^{L1c} is —CH₂— or —C(O)—
j is an integer between 0 and 20
k is an integer between 1 and 8; and
o is an integer between 1 and 20.

44. The compound or pharmaceutically acceptable salt of claim 43, wherein R^{L1b} is —H.

45. The compound or pharmaceutically acceptable salt of claim 43, wherein R^{L1b} is —CO₂H.

46. The compound or pharmaceutically acceptable salt of any one of claims 43-45, wherein R^{L1c} is —CH₂—.

47. The compound or pharmaceutically acceptable salt of any one of claims 43-45, wherein R^{L1c} is —C(O)—.

48. The compound or pharmaceutically acceptable salt of any one of claims 43-47, wherein j is 0, 1, 4, or 7.

49. The compound or pharmaceutically acceptable salt of any one of claims 43-48, wherein k is 1, 2, 3, 4, or 5.

50. The compound or pharmaceutically acceptable salt of any one of claims 43-49, wherein o is 2, 3, 6, or 10.

51. The compound or pharmaceutically acceptable salt of any one of claims 1-50, wherein at least one instance of L¹ is independently of the formula:

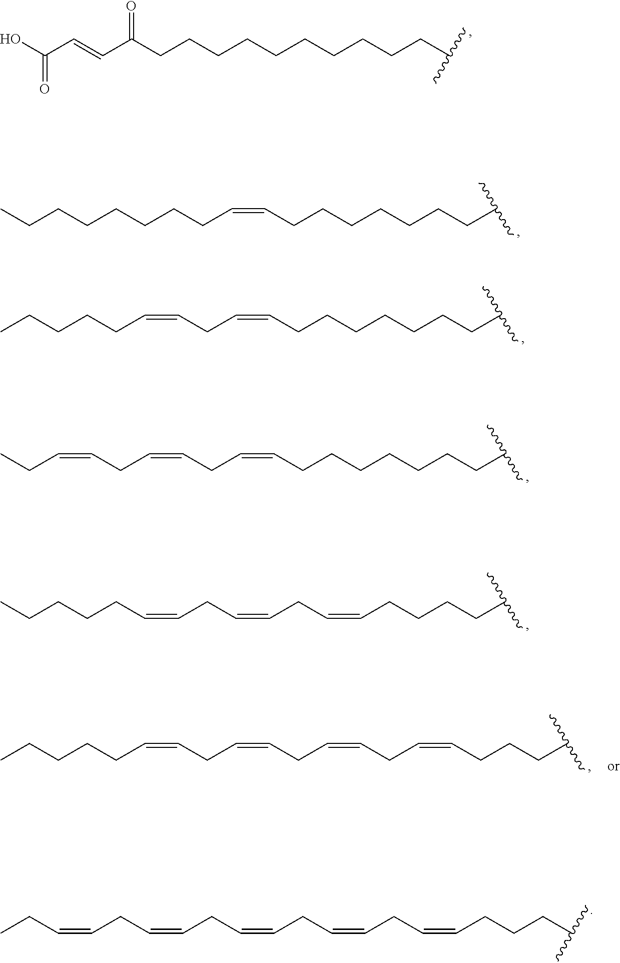

52. The compound or pharmaceutically acceptable salt of any one of claims 1-51, wherein Y¹ is a bond.

53. The compound or pharmaceutically acceptable salt of any one of claims 1-52, wherein Y¹ is a linker connecting at least one L⁴ to L³ if present, or L⁴ to R^{Z3}.

54. The compound or pharmaceutically acceptable salt of claims 1-53, wherein Y¹ is a bivalent moiety connecting at least one L⁴ to L³ if present, or connecting L⁴ to R^{Z3}.

55. The compound or pharmaceutically acceptable salt of any one of claims 1-54, wherein Y¹ is of the formula: 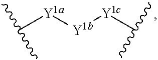
wherein:
Y^{1a} and Y^{1c} are each independently absent, —N(H)—, or —C(O)—;
Y^{1b} is absent or substituted or unsubstituted heteroalkylene, substituted or unsubstituted carbocyclene, substituted or unsubstituted heterocyclene; substituted or unsubstituted arylene, or substituted or unsubstituted heteroarylene.

56. The compound or pharmaceutically acceptable salt of claim 55, wherein Y^{1a} is —C(O)—.

57. The compound or pharmaceutically acceptable salt of claim 55, wherein Y^{1a} is —N(H)—.

58. The compound or pharmaceutically acceptable of any one of claims 55-57, wherein Y^{1c} is —(CO)—.

59. The compound or pharmaceutically acceptable salt of any one of claims 55-57, wherein Y^{1c} is —N(H)—.

60. The compound or pharmaceutically acceptable salt of any one of claims 55-59, wherein Y^{1b} is substituted or unsubstituted C₁-C₆ heteroalkylene.

61. The compound or pharmaceutically acceptable salt of any one of claims 55-60, wherein Y^{1b} is of the formula

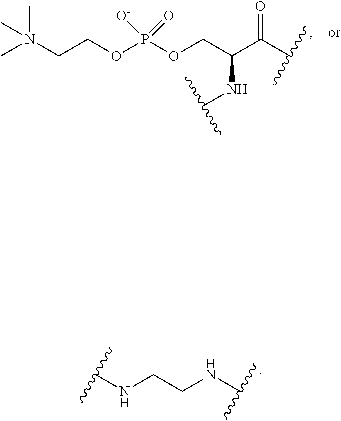

62. The compound or pharmaceutically acceptable salt of any one of claims 55-59, wherein Y^{1b} substituted or unsubstituted carbocyclene.

63. The compound or pharmaceutically acceptable salt of any one of claims 55-59, or 62, wherein Y^{1b} is of the formula

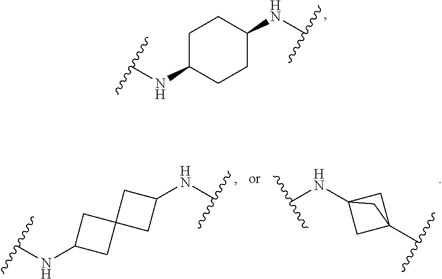 [SMILES: *N[C@H]1CC[C@@H](N*)CC1 ; *NC1CC2(C1)CC(N*)C2 ; *NC12CC(*)(C1)C2]

64. The compound or pharmaceutically acceptable salt of any one of claims 55-59, wherein Y^{1b} substituted or unsubstituted heterocyclene.

65. The compound or pharmaceutically acceptable salt of any one of claims 55-59, or 64, wherein Y^{1b} is of the formula

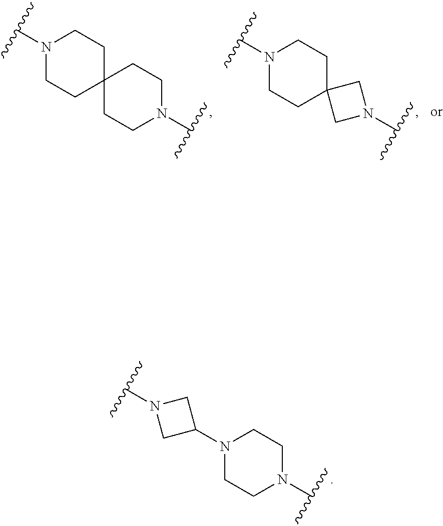 [SMILES: *N1CCC2(CC1)CCN(*)CC2 ; *N1CCC2(CC1)CN(*)C2 ; *N1CCN(C2CN(*)C2)CC1]

66. The compound or pharmaceutically acceptable salt of any one of claims 55-59, wherein Y^{1b} substituted or unsubstituted arylene.

67. The compound or pharmaceutically acceptable salt of any one of claims 55-59, or 66, wherein Y^{1b} substituted or unsubstituted phenylene.

68. The compound or pharmaceutically acceptable salt of any one of claims 55-59, or 66-67, wherein Y^{1b} is of the formula

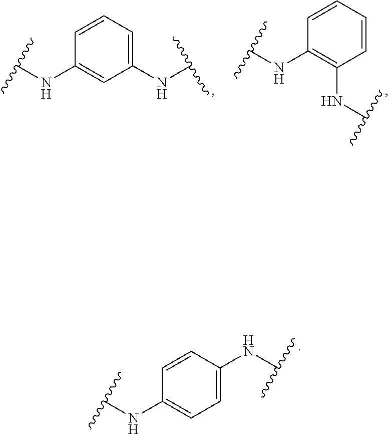 [SMILES: *Nc1cccc(N*)c1 ; *Nc1ccccc1N* ; *Nc1ccc(N*)cc1]

69. The compound or pharmaceutically acceptable salt of any one of claims 1-68, wherein Y¹ is selected from the group consisting of —N(H)—C(O)—, —C(O)—N(H)—,

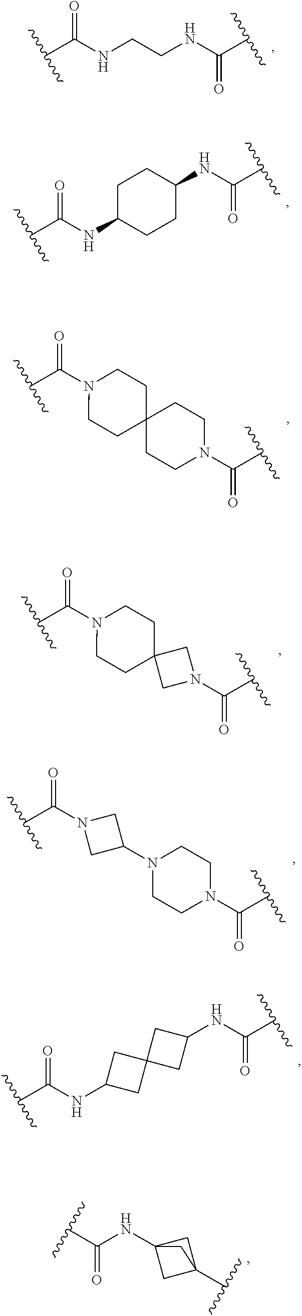

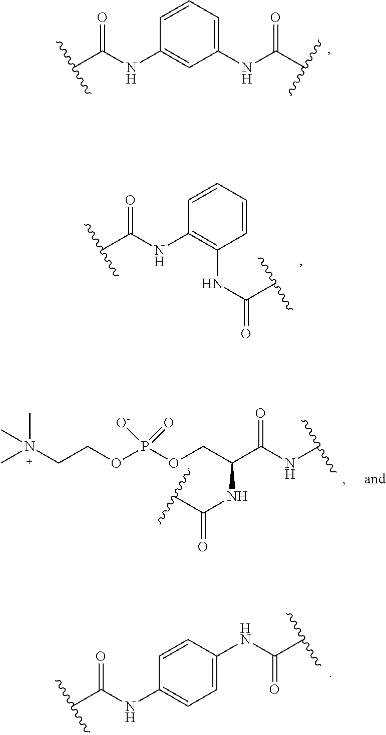

70. The compound or pharmaceutically acceptable salt of any one of claims 1-51, wherein Y¹ is a trivalent moiety connecting two L⁴ groups to L³, or connecting two L⁴ groups to R^{Z3}.

71. The compound or pharmaceutically acceptable salt of claim 70, wherein Y¹ is of the formula:

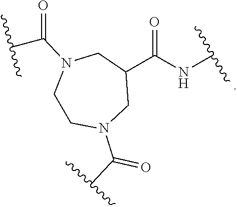 [SMILES: *NC(=O)C1CN(C(*)=O)CCN(C(*)=O)C1]

72. The compound or pharmaceutically acceptable salt of any one of claims 1-71, wherein L³ is of the formula 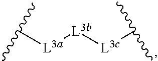
wherein:
L^{3a} is a bond or a bidentate linking group;
L^{3b} is a bond or a chemical moiety formed by reacting a first reactive moiety with a second reactive moiety; and
L^{3c} is a bond or of the formula: 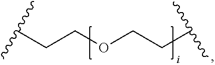
wherein i is an integer between 1 and 12.

73. The compound or pharmaceutically acceptable salt of claim 72, wherein L^{3b} is a bond, —C(O)—, or of the formula:

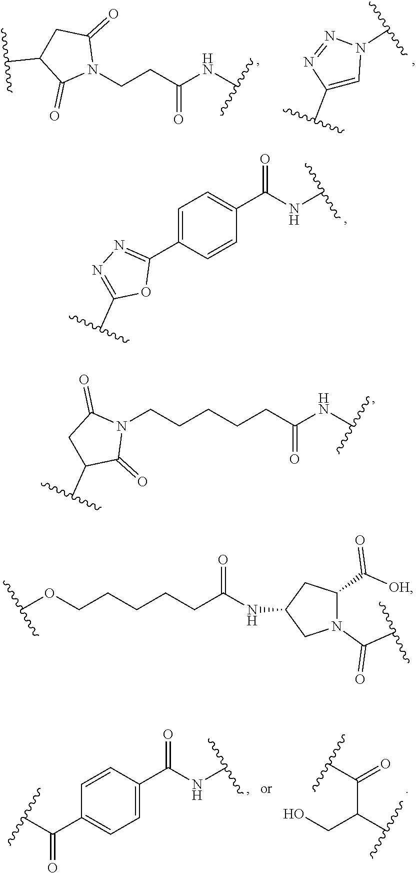

74. The compound or pharmaceutically acceptable salt of claim 72 or 73, wherein L^{3a} is of the formula:

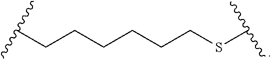 [SMILES: *CCCCCCS*]

75. The compound or pharmaceutically acceptable salt of any one of claims 1-74, wherein at least one instance of L⁴ is independently a saturated lipid.

76. The compound or pharmaceutically acceptable salt of any one of claims 1-75, wherein at least one instance of L⁴ is independently a straight chain lipid.

77. The compound or pharmaceutically acceptable salt of any one of claims 1-76, wherein at least one instance of L⁴ is independently of the formula: 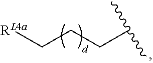
wherein
R^{L4a} is H, or CO₂H; and
d is an integer between 5 and 35.

78. The compound or pharmaceutically acceptable salt of claim 77, wherein R^{L4a} is H.

79. The compound or pharmaceutically acceptable salt of claim 77 or 78, wherein R^{L4a} is CO₂H.

80. The compound or pharmaceutically acceptable salt of any one of claims 77-79, wherein d is 8, 9, 10, 11, 12, 13, 14, 15, 16, 17, 18, 19, or 20.

81. The compound or pharmaceutically acceptable salt of any one of claims 77-80, wherein d is 9, 11, 12, 13, 14, 15, or 17.

82. The compound or pharmaceutically acceptable salt of claim 77, wherein d is 13.

83. The compound or pharmaceutically acceptable salt of claim 82, wherein R^{L4a} is H.

84. The compound or pharmaceutically acceptable salt of claim 77, wherein d is 12.

85. The compound or pharmaceutically acceptable salt of claim 84, wherein R^{L4a} is CO₂H.

86. The compound or pharmaceutically acceptable salt of any one of claims 1-85, wherein at least one instance of L⁴ is independently a branched lipid.

87. The compound or pharmaceutically acceptable salt of any one of claims 1-86, wherein at least one instance of L⁴ is independently of the formula 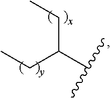
wherein:
x is an integer between 2 and 25; and
y is an integer between 2 and 25.

88. The compound or pharmaceutically acceptable salt of any one of claims 1-87, wherein at least one instance of L⁴ is independently of the formula:

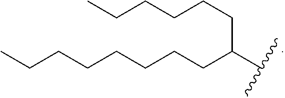 [SMILES: *C(CCCCCC)CCCCCCCC]

89. The compound or pharmaceutically acceptable salt of any one of claims 1-88, wherein at least one instance of L⁴ is independently an unsaturated lipid.

90. The compound or pharmaceutically acceptable salt of any one of claims 1-89, wherein at least one instance of L⁴ is independently a straight chain lipid comprising 1-8 alkenylene moieties.

91. The compound or pharmaceutically acceptable salt of any one of claims 1-90, wherein at least one instance of L⁴ is independently of the formula 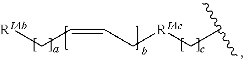
wherein:
R^{L4b} is —CH₃, or —CO₂H;
R^{L4c} is —CH₂— or —C(O)—
a is an integer between 0 and 20
b is an integer between 1 and 8; and
c is an integer between 1 and 20.

92. The compound or pharmaceutically acceptable salt of claim 91, wherein R^{L4b} is —H,

93. The compound or pharmaceutically acceptable salt of claim 91, wherein R^{L4b} is —CO₂H.

94. The compound or pharmaceutically acceptable salt of any one of claims 91-93, wherein R^{L4c} is —CH₂—.

95. The compound or pharmaceutically acceptable salt of any one of claims 91-93, wherein R^{L4c} is —C(O)—.

96. The compound or pharmaceutically acceptable salt of any one of claims 91-95, wherein a is 0, 1, 4, or 7.

97. The compound or pharmaceutically acceptable salt of any one of claims 91-96, wherein b is 1, 2, 3, 4, or 5.

98. The compound or pharmaceutically acceptable salt of any one of claims 91-97, wherein c is 2, 3, 6, or 10.

99. The compound or pharmaceutically acceptable salt of any one of claims 1-98, wherein at least one instance of L⁴ is independently of the formula:

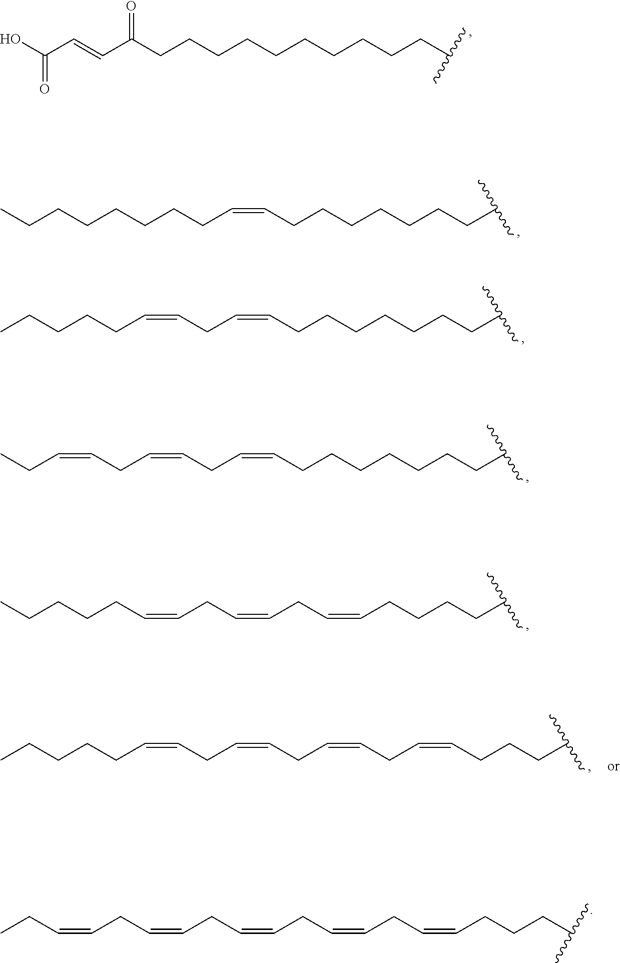

100. The compound or pharmaceutically acceptable salt of any one of claims 1-99, wherein 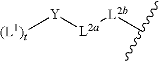
is selected from the group consisting of any of the PK/PD modulators shown in Table 2.

101. The compound or pharmaceutically acceptable salt of any one of claims 1-100, wherein 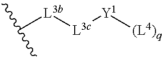
is selected from the group consisting of any of the PK/PD modulators shown in Table 2.

102. The compound or pharmaceutically acceptable salt of any one of claims 1-101, wherein R^{Z1} is an inverted abasic residue.

103. The compound or pharmaceutically acceptable salt of any one of claims 1-102, wherein R^{Z1} is of the formula:

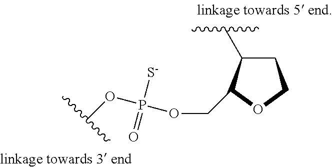

104. The compound or pharmaceutically acceptable salt of any one of claims 1-103, wherein R^{Z3} is an inverted abasic residue.

105. The compound or pharmaceutically acceptable salt of any one of claims 1-104, wherein R^{Z3} is of the formula:

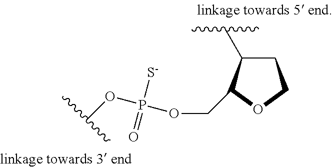

106. A compound comprising:
a) an oligonucleotide-based agent comprising an oligonucleotide; and
b) at least one lipid conjugated to the oligonucleotide-based agent, wherein the at least one lipid is conjugated to the 5′ terminus or the 3′ terminus of the oligonucleotide;
wherein the oligonucleotide-based agent comprises at least 15 nucleotides that are complementary to a gene expressed in adipose tissue.

107. The compound of claim 106, wherein the oligonucleotide-based agent is double-stranded.

108. The compound of claim 106 or 107, wherein the oligonucleotide-based agent comprises a sense strand and an antisense strand.

109. The compound of claim 108, wherein the at least one lipid is conjugated to the sense strand.

110. The compound of claim 109, wherein the at least one lipid is conjugated to the 5′ position of the sense strand.

111. The compound of any one of claims 106-110, wherein the at least one lipid is saturated.

112. The compound of any one of claims 106-110, wherein the at least one lipid is unsaturated.

113. The compound of any one of claims 106-112, wherein the at least one lipid comprises 10 to 30 carbon atoms.

114. The compound of any one of claims 106-113, wherein the at least one lipid comprises 15 to 20 carbon atoms.

115. The compound of any one of claims 106-114, wherein the at least one lipid is selected from the group consisting of: 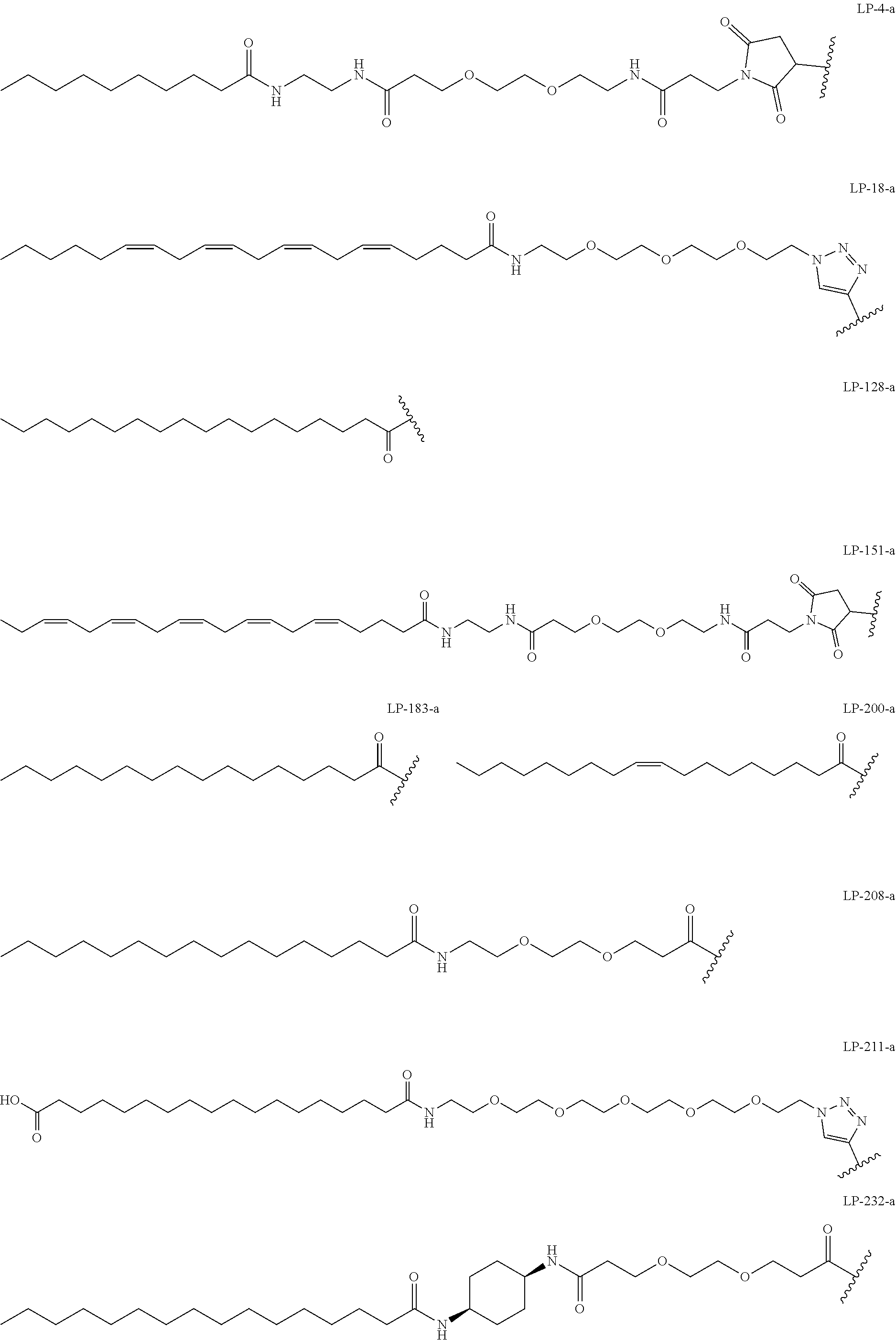 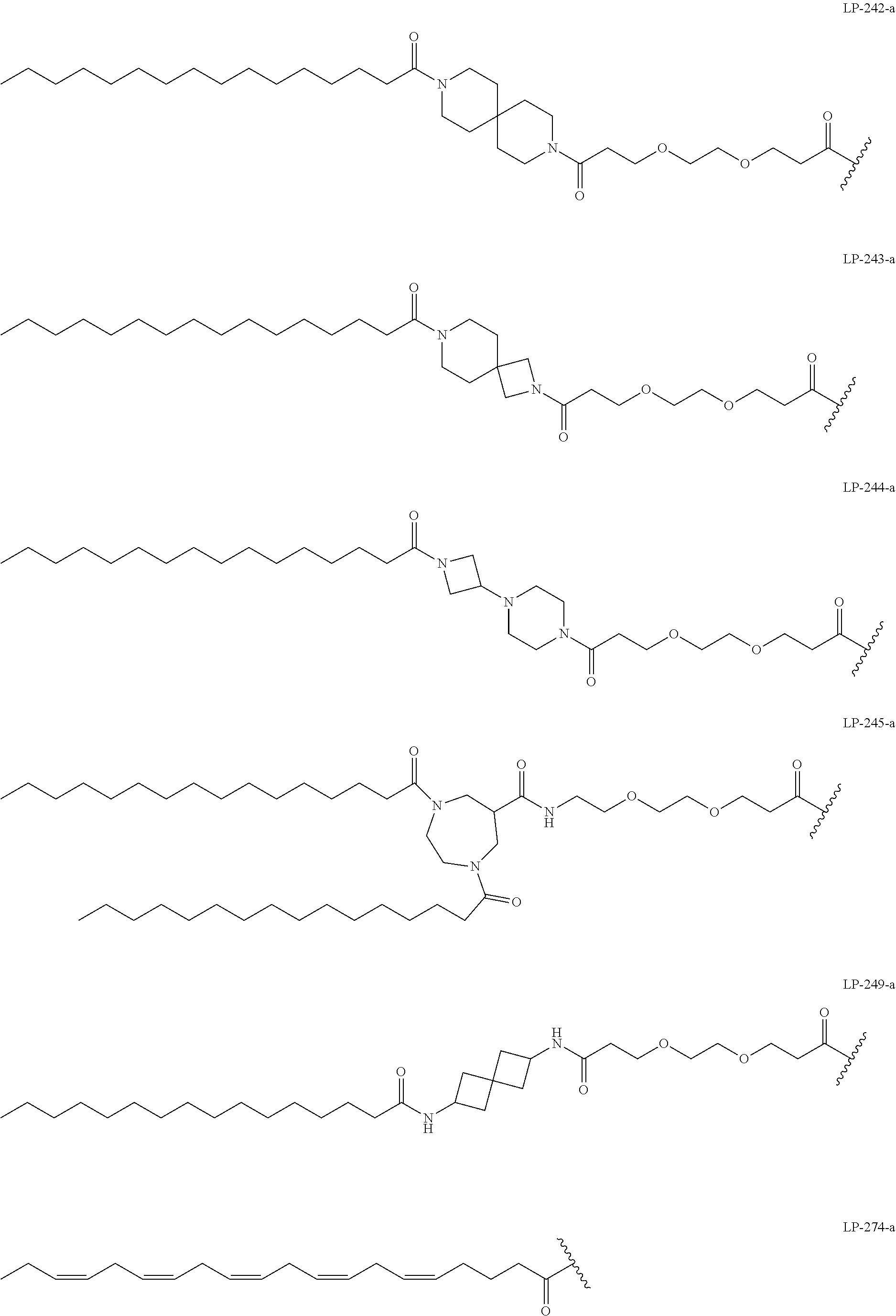 [SMILES: *C(=O)CCOCCOCCC(=O)N1CCC2(CCN(C(=O)CCCCCCCCCCCCCCC)CC2)CC1 ; *C(=O)CCOCCOCCC(=O)N1CC2(CCN(C(=O)CCCCCCCCCCCCCCC)CC2)C1 ; *C(=O)CCOCCOCCC(=O)N1CCN(C2CN(C(=O)CCCCCCCCCCCCCCC)C2)CC1 ; *C(=O)CCOCCOCCNC(=O)C1CN(C(=O)CCCCCCCCCCCCCCC)CCN(C(=O)CCCCCCCCCCCCCCC)C1 ; *C(=O)CCOCCOCCC(=O)NC1CC2(CC(NC(=O)CCCCCCCCCCCCCCC)C2)C1 ; *C(=O)CCC/C=C\C/C=C\C/C=C\C/C=C\C/C=C\CC] 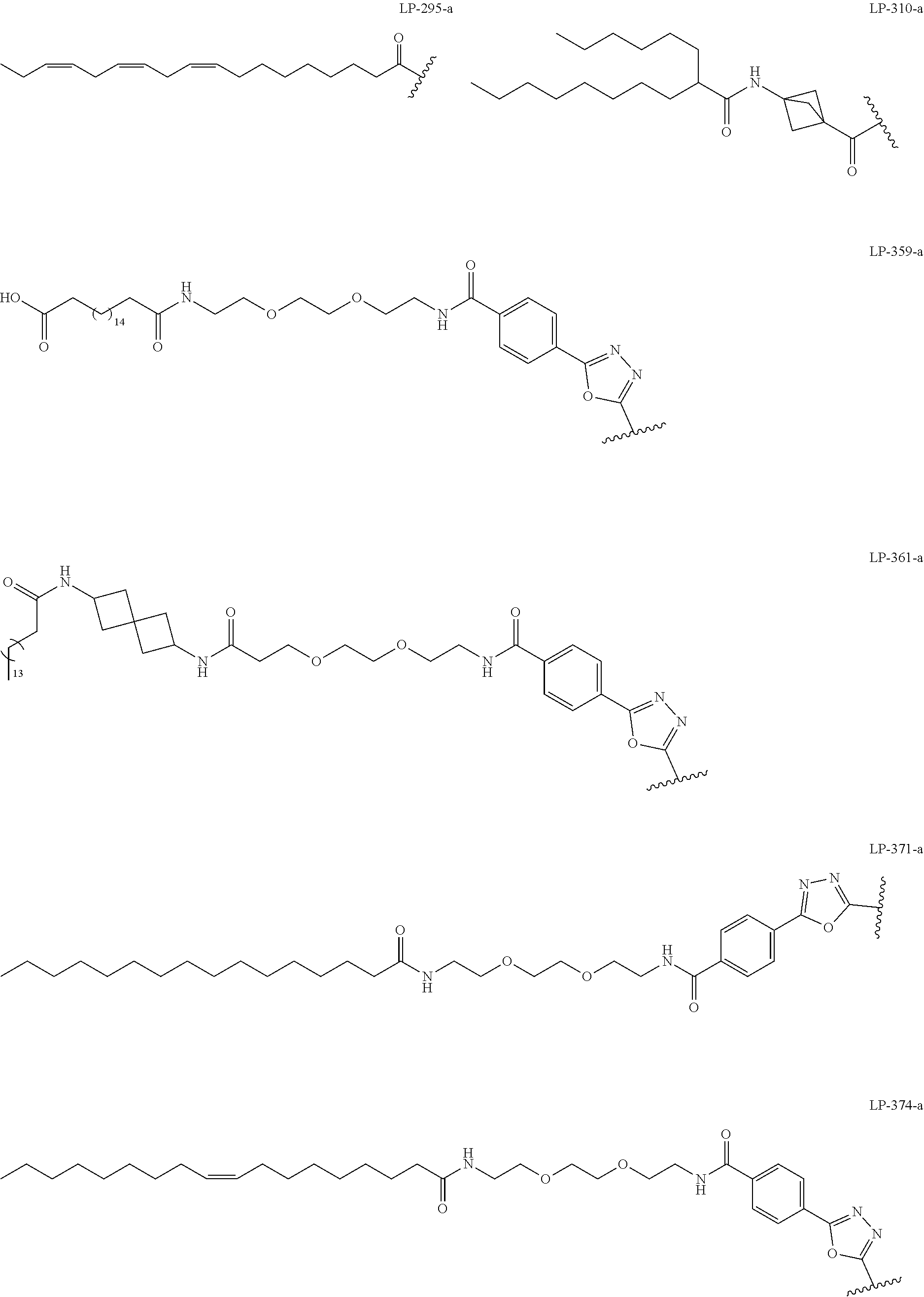 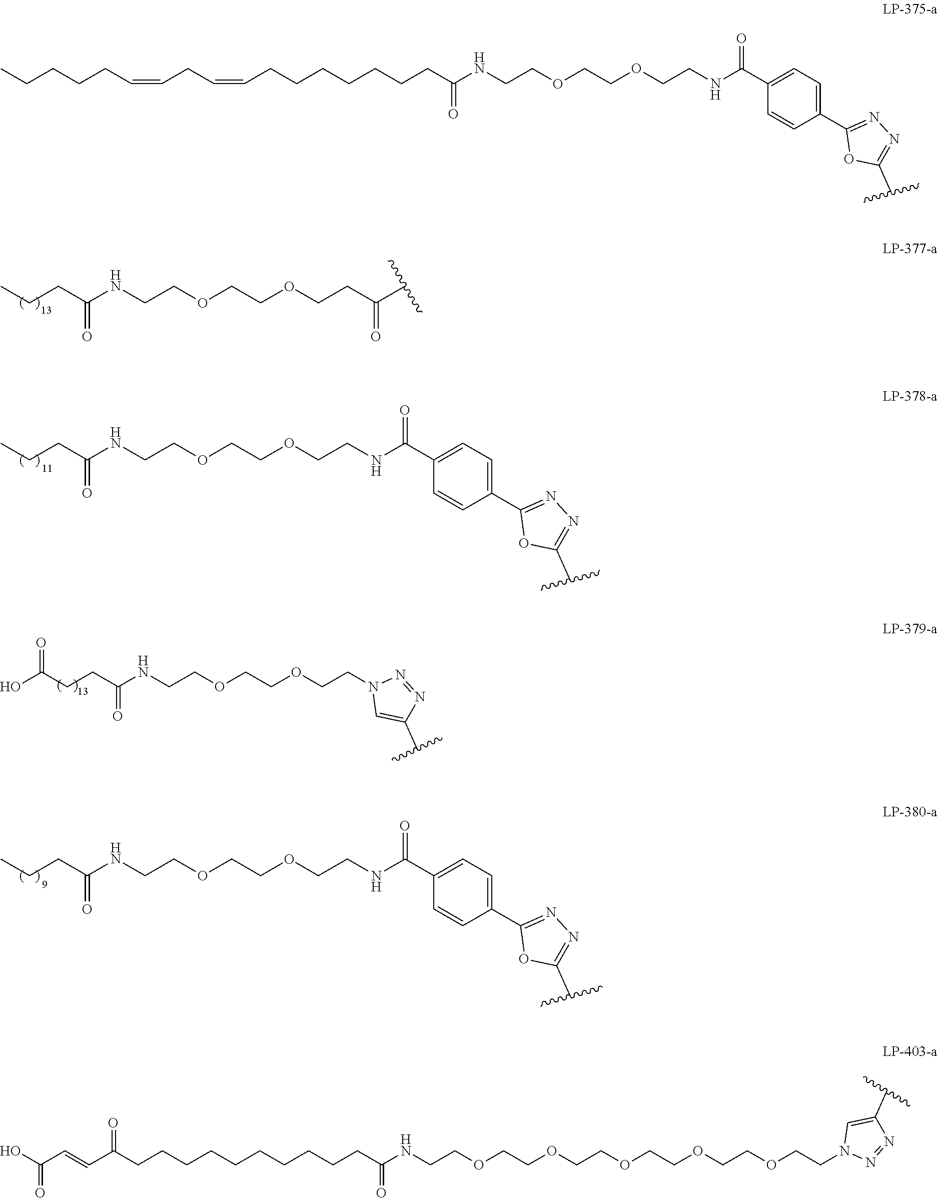 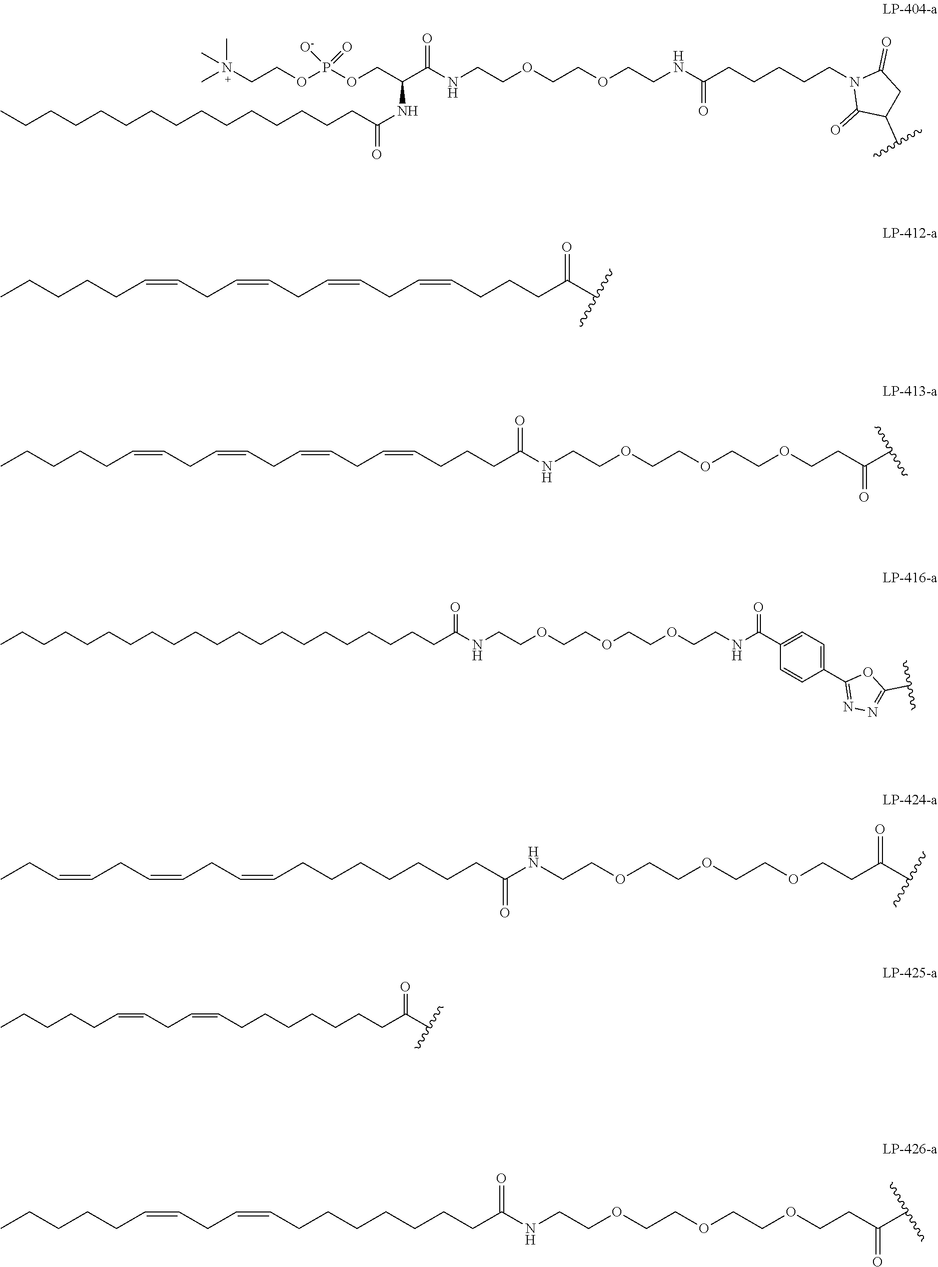 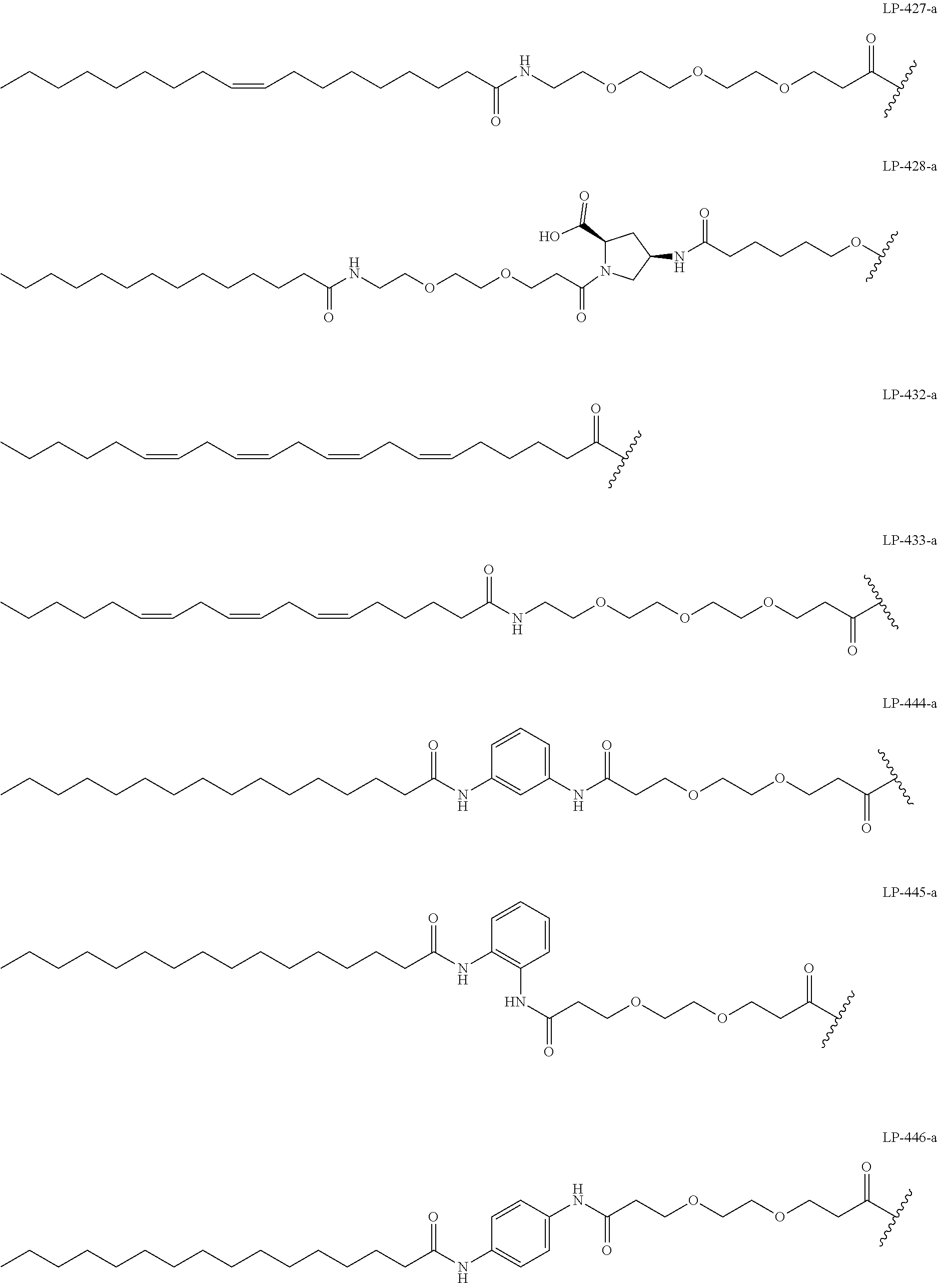 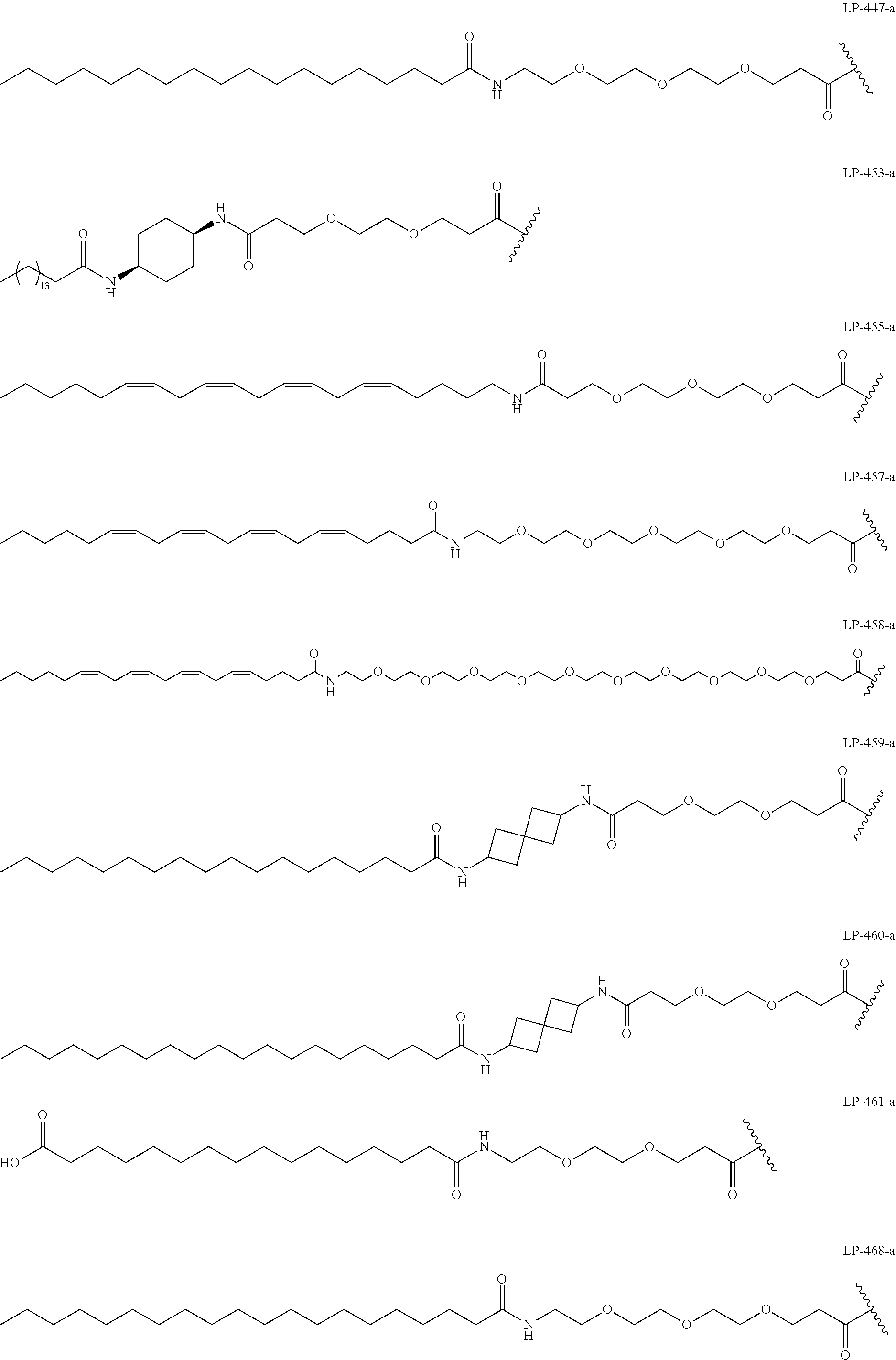 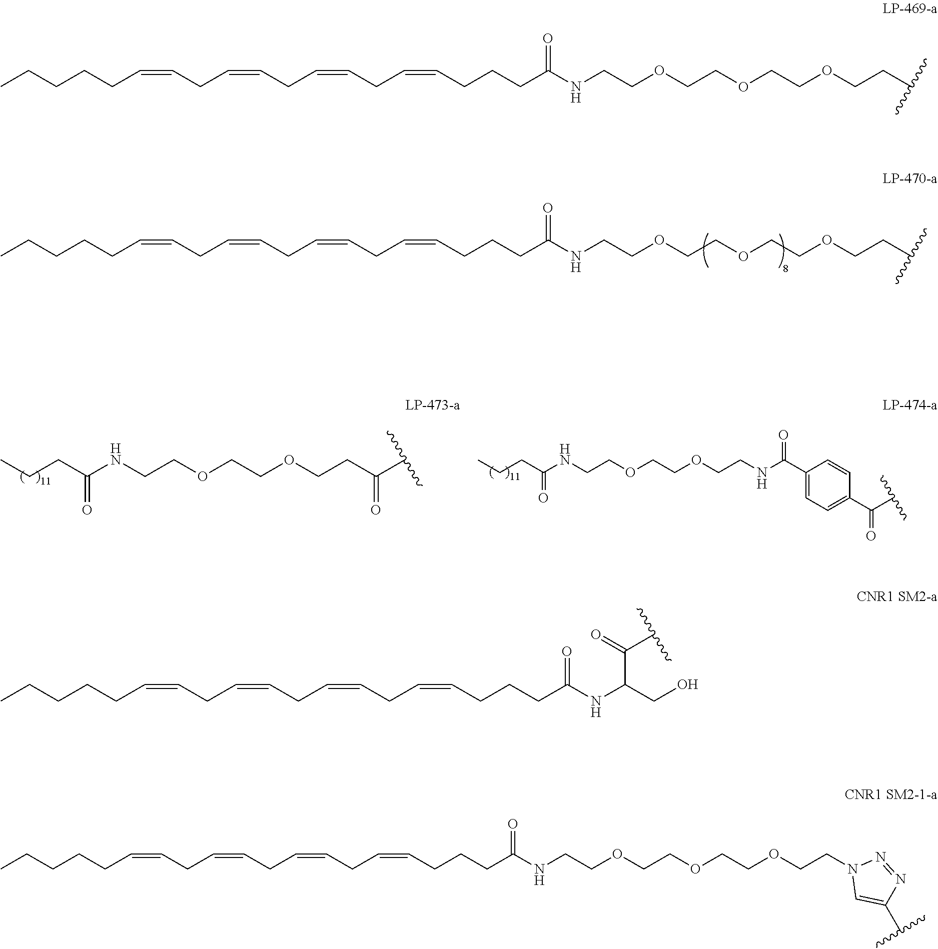
wherein [symbol] indicates the point of connection to the oligonucleotide.

116. The compound of any one of claims 106-115, wherein the oligonucleotide based agent is conjugated to at least two lipids.

117. The compound of any one of claims 106-116, wherein the oligonucleotide-based agent is conjugated to at least three lipids.

118. The compound of claim 116, wherein the at least two lipids are conjugated to the 5′ terminus of the oligonucleotide and the 3′ terminus of the oligonucleotide.

119. The compound of any one of claims 106-118, wherein the oligonucleotide-based agent is an RNAi agent wherein the antisense strand comprises a cyclopropyl-phosphonate-modified nucleotide (cPrp) or a vinyl-phosphonate modified nucleotide (vp).

120. The compound of claim 119, wherein the cyclopropyl-phosphonate modified nucleotide or vinyl-phosphonate modified nucleotide is the 5′-terminal nucleotide of the antisense strand.

121. A compound selected from the group consisting of: 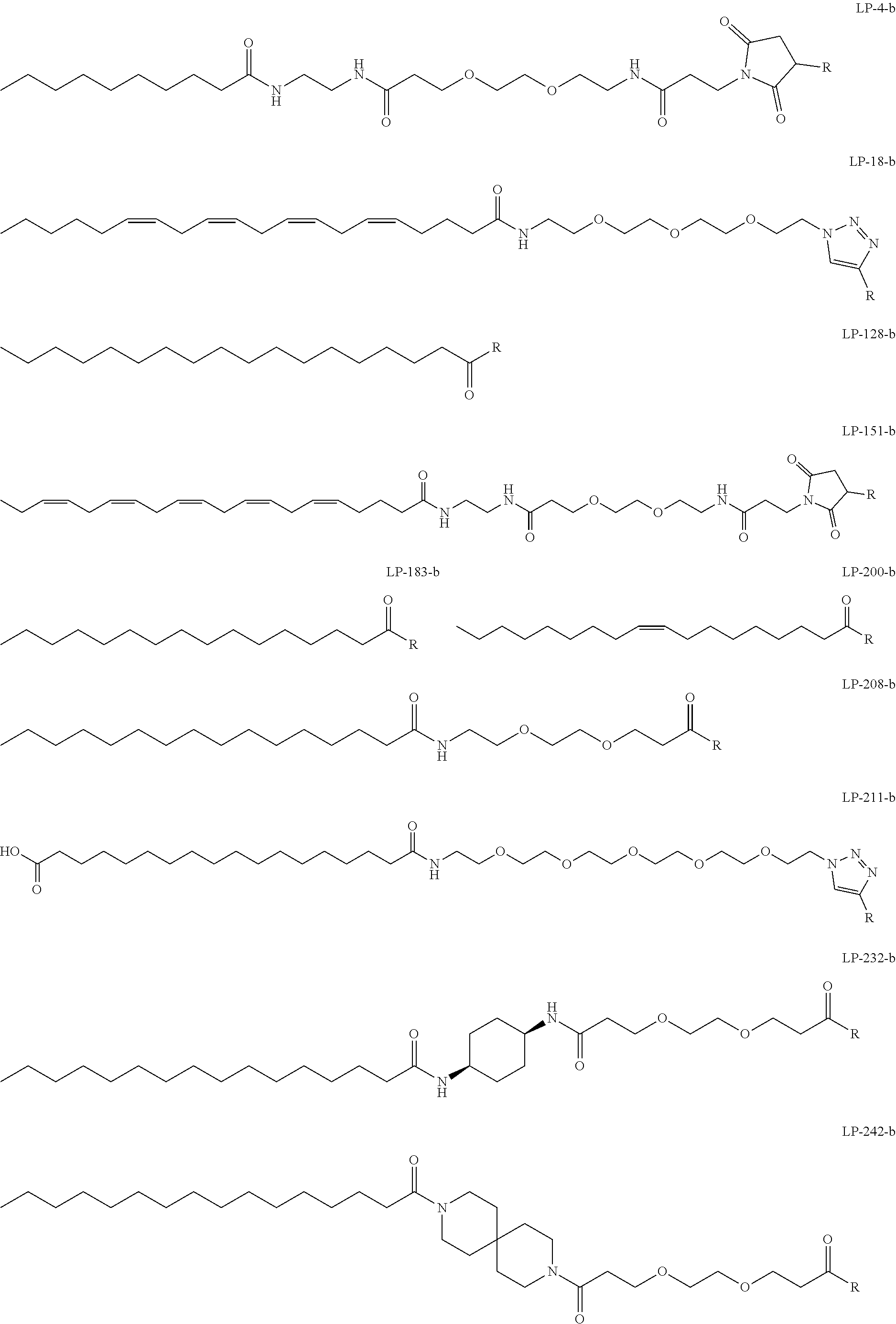 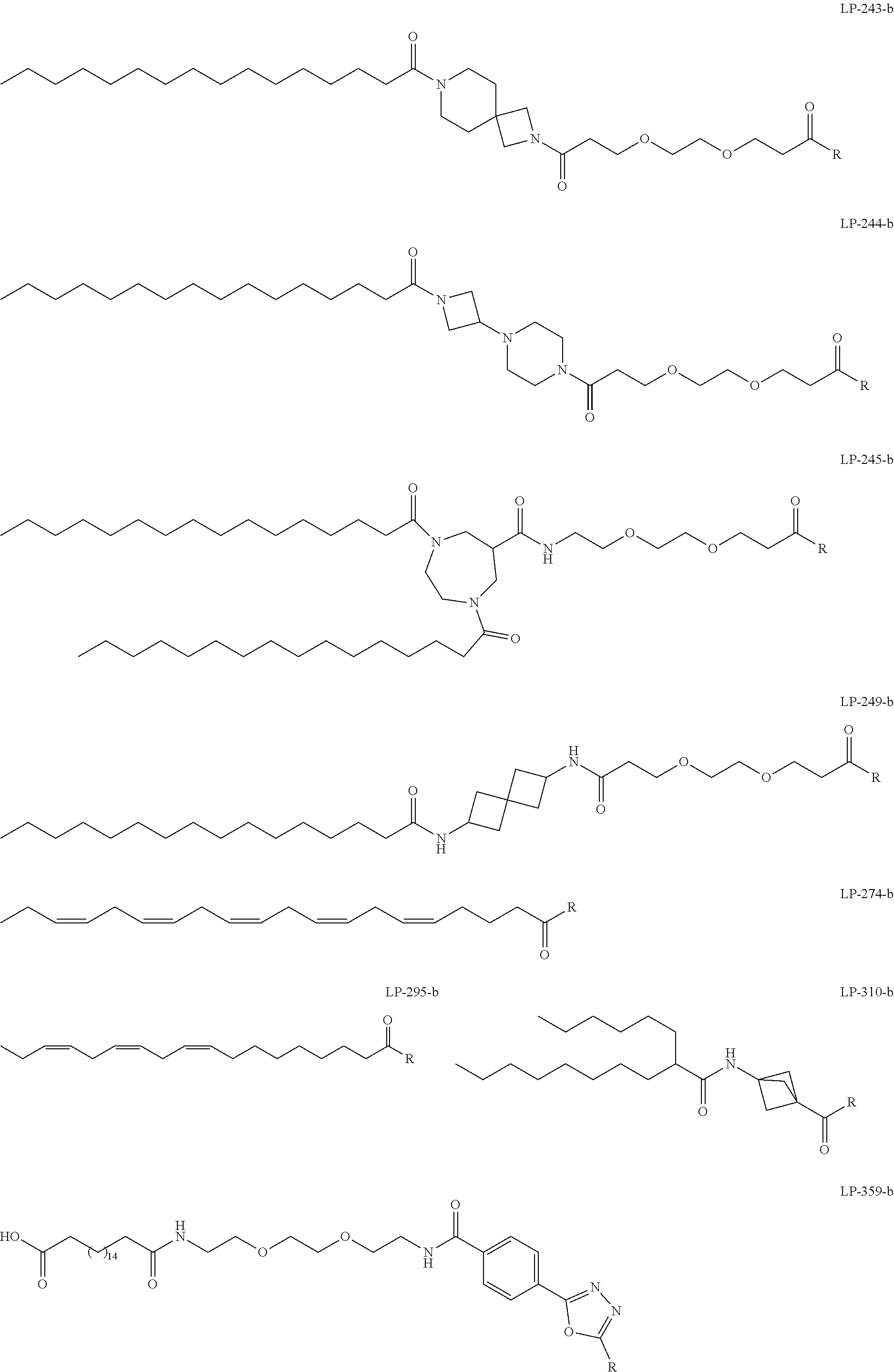 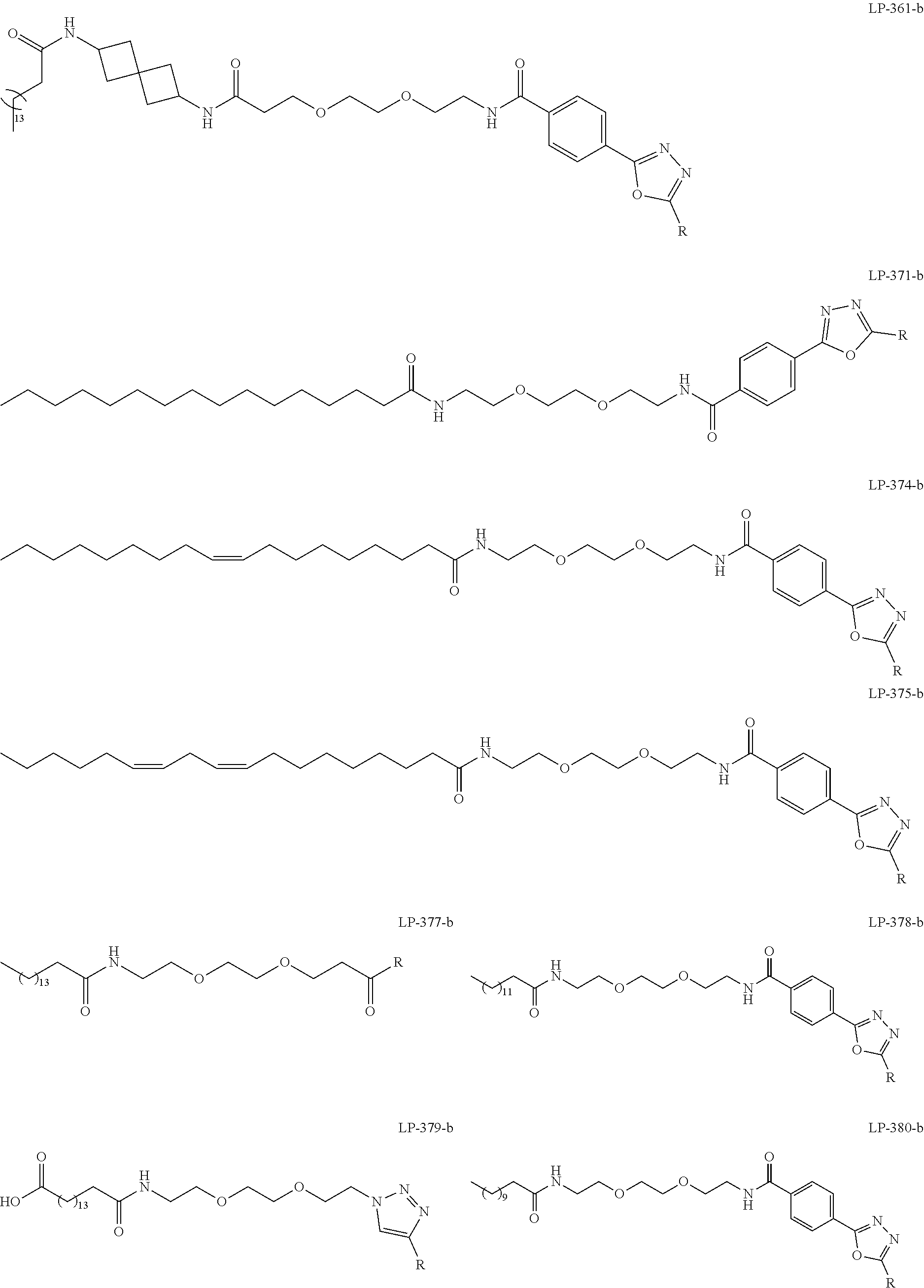 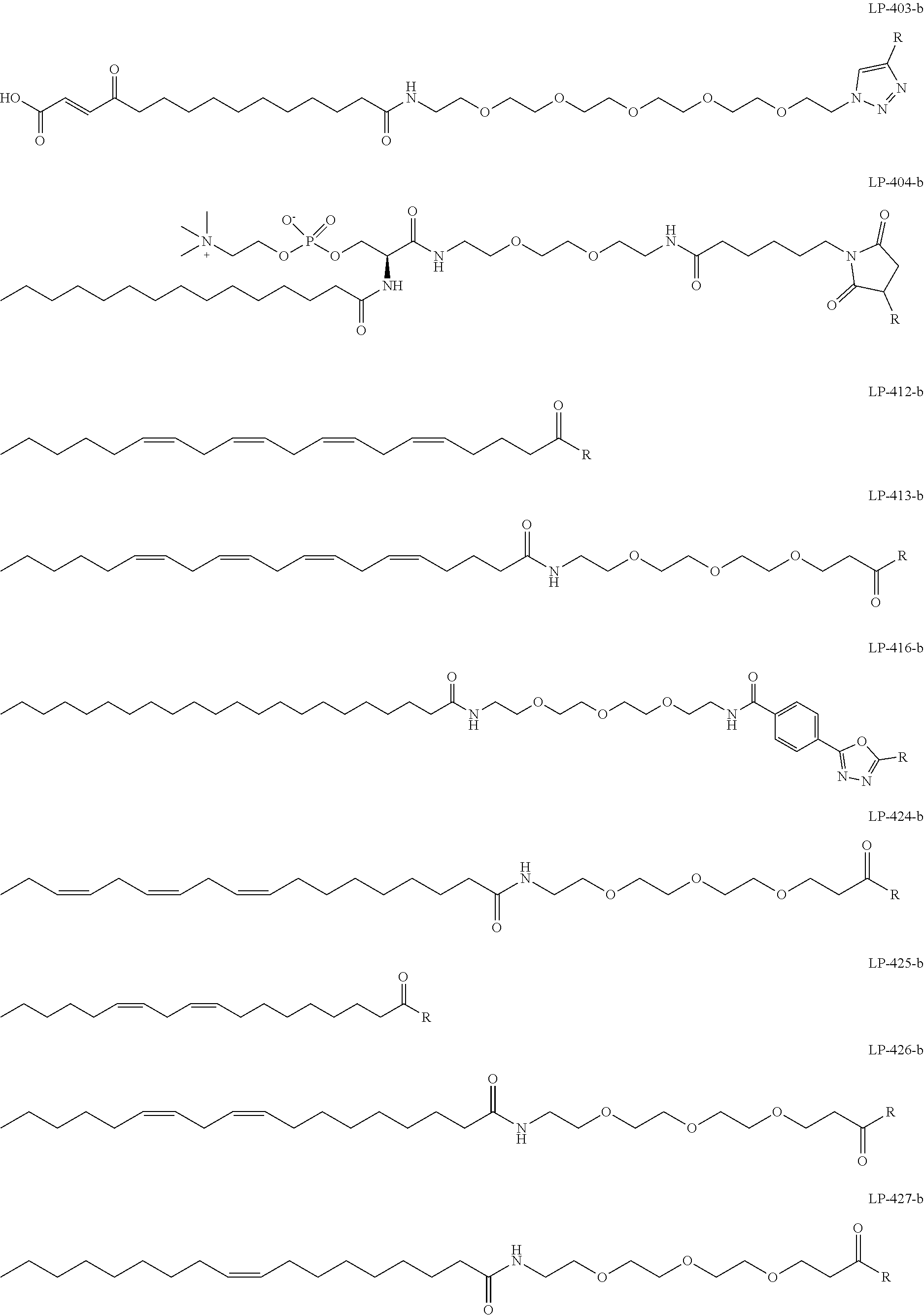 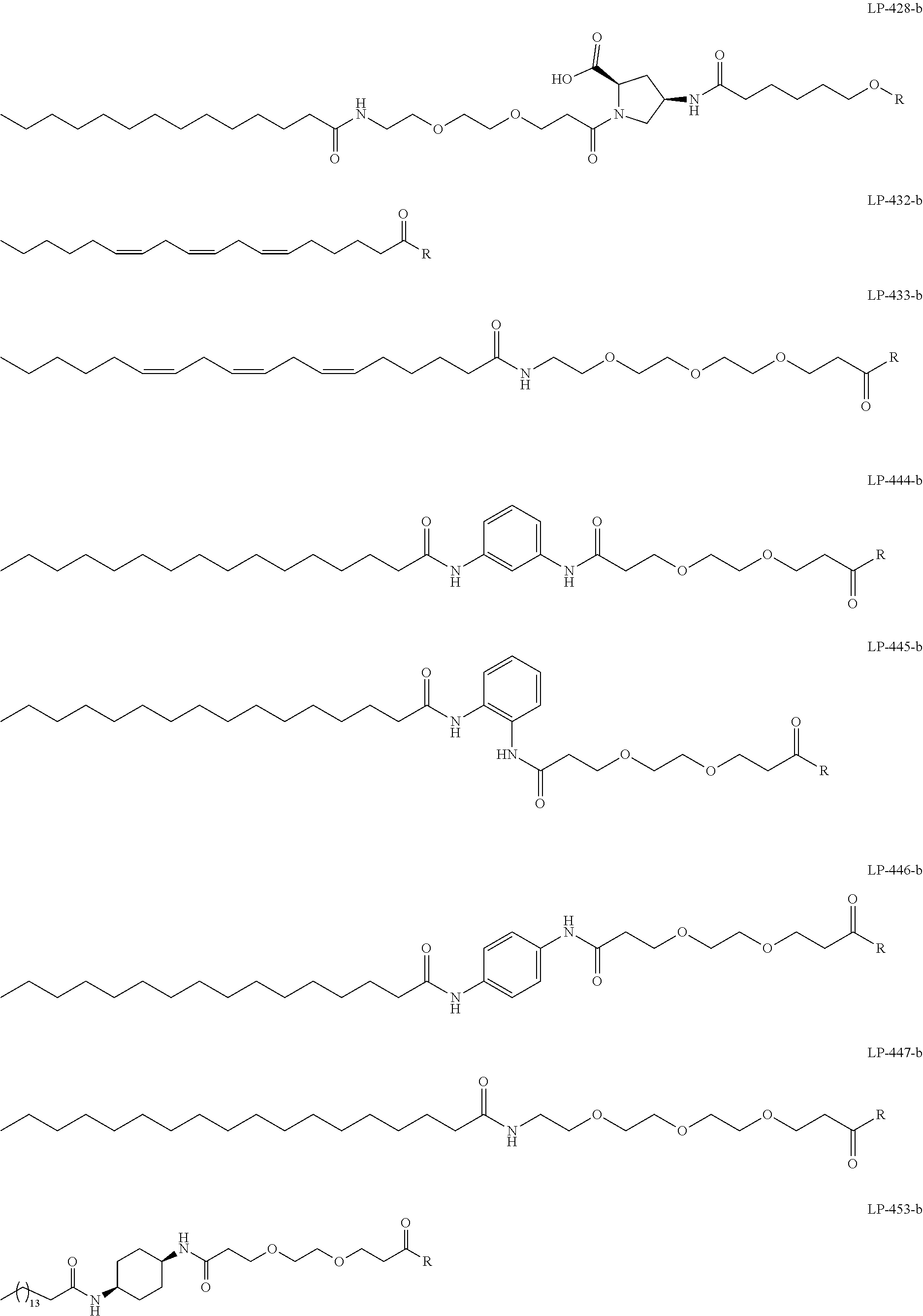 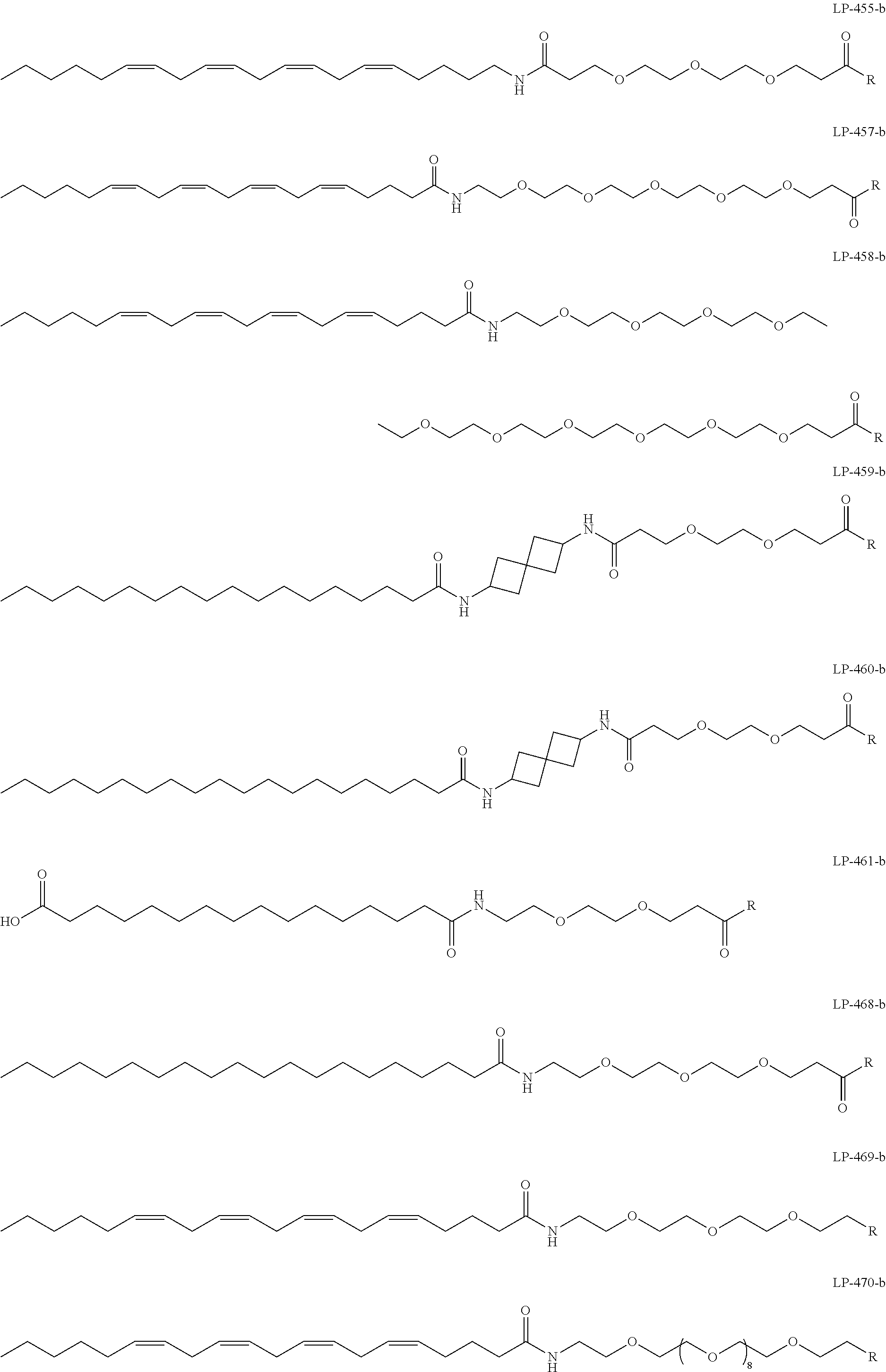 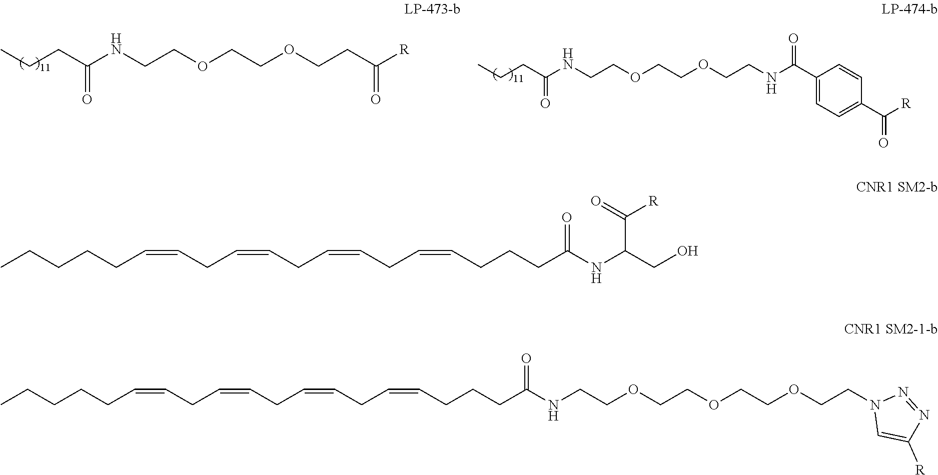
wherein R comprises an oligonucleotide.

122. The compound of claim 121, wherein the oligonucleotide is double stranded.

123. The compound of claim 122, wherein the oligonucleotide comprises a sense strand and an antisense strand.

124. The compound of claim 123, wherein the point of connection to R is on the sense strand.

125. The compound of claim 124, wherein the point of connection to R is on the 5′ terminal nucleotide of the sense strand.

126. An RNAi agent comprising a sense strand and an antisense strand, wherein:
the sense strand is a compound of any one of claims 1-125, and the antisense strand is at least 70%, 85%, or 90% complimentary to the sense strand.

127. The RNAi agent of claim 126, wherein the antisense strand is at least 70%, 80%, or 90% complementary to the mRNA of a gene expressed in adipose tissue.

128. The RNAi agent of claim 126 or 127, wherein the antisense strand is at least 70%, 80%, or 90% complementary to the mRNA of a gene expressed in human adipose tissue.

129. The RNAi agent of any one of claims 126-128, wherein the antisense strand is at least 70%, 80%, or 90% complementary to the mRNA of a gene expressed in an adipocyte.

130. The RNAi agent of any one of claims 126-129, wherein the antisense strand is at least 70%, 80%, or 90% complementary to the mRNA of a gene expressed in a human adipocyte.

131. The RNAi agent of claim 129 or 130, wherein the gene is expressed in a white adipocyte cell.

132. The RNAi agent of any one of claims 129-131, wherein the gene is expressed in a mature adipocyte.

133. A pharmaceutical composition comprising a compound or pharmaceutically acceptable salt thereof, of any one of claims 1-125, or the RNAi agent of any one of claims 126-132, and a pharmaceutically acceptable excipient.

134. A method for delivering an oligonucleotide to adipose tissue, comprising administering to a subject a compound of any one of claims 1-125, an RNAi agent of any one of claims 123-132, or a pharmaceutical composition of claim 133.

135. A method for delivering an oligonucleotide to a cell, comprising administering to a subject a compound of any one of claims 1-125, an RNAi agent of any one of claims 126-132, or a pharmaceutical composition of claim 133.

136. The method of claim 135, wherein the cell is an adipocyte.

137. A method of modulating the activity of a gene in a subject, biological sample, or cell, the method comprising introducing into the subject, biological sample, or cell an effective amount of a compound of any one of claims 1-125, an RNAi agent of any one of claims 126-132, or a pharmaceutical composition of claim 133, wherein the gene is a gene expressed in adipose tissue or an adipocyte.

138. The method of 137, wherein the expression of the gene is inhibited.

139. The method of 137 or 138, wherein the expression is inhibited by at least 50%, 60%, 70%, 80%, or 90% compared to expression prior to administration.

140. The method of any one of claims 137-139, wherein the gene is overexpressed in the subject, biological sample, or cell.

141. The method of any one of claims 137-140, wherein the gene is expressed in a white adipocyte cell.

142. The method of any one of claims 137-141, wherein the gene is expressed in a mature adipocyte.

143. A compound of Formula (II): 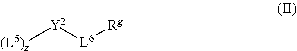
or a pharmaceutically acceptable salt thereof, wherein
R^{g} is a reactive moiety suitable for conjugation with an oligonucleotide-based agent;
Y² is a bond or a linker connecting at least one L⁵ to L⁶ when present, or to R^{g};
each L⁵ is independently a lipid comprising from about 10 to about 50 carbon atoms
L⁶ is a linker comprising 1-20 PEG units; and
z is 1, 2, or 3, as valency permits.

144. The compound or pharmaceutically acceptable salt of claim 143, wherein Y² is a bond.

145. The compound or pharmaceutically acceptable salt of claim 143, wherein Y² is a linker connecting at least one L⁵ to L⁶ if present, or L⁵ to R^{g}.

146. The compound or pharmaceutically acceptable salt of any one of claims 143-145 wherein Y² is a bivalent moiety connecting at least one L⁵ to L⁶ if present, or connecting L⁵ to R^{g}.

147. The compound or pharmaceutically acceptable salt of claim 145 or 146, wherein Y² is selected from the group consisting of —N(H)—C(O)—, —C(O)—N(H)—,

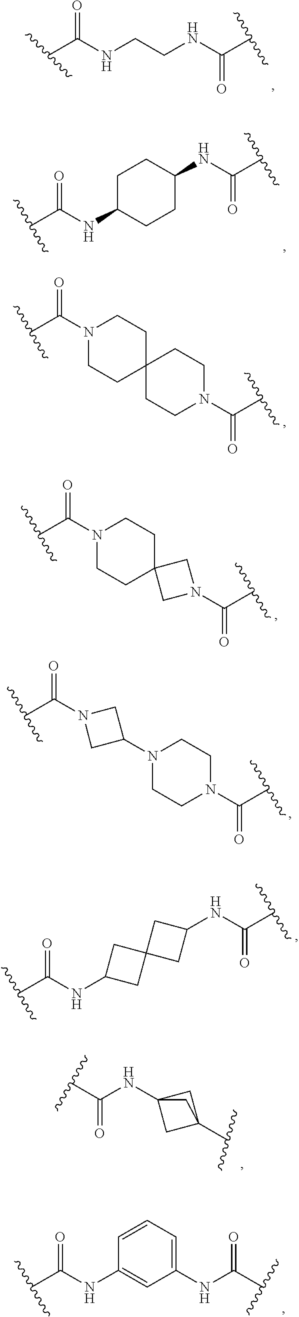

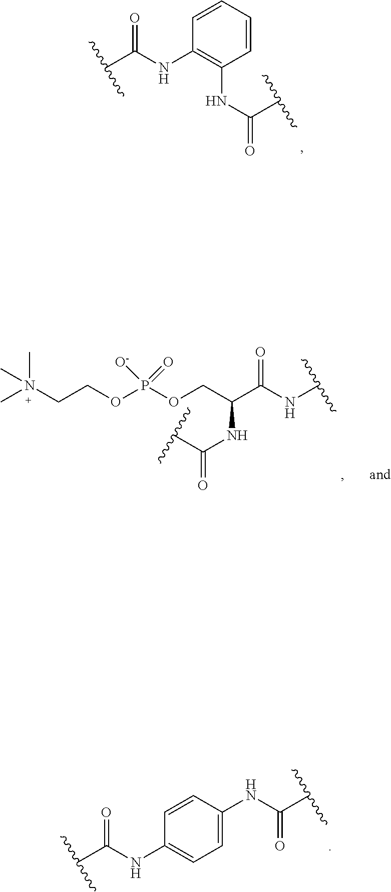

148. The compound or pharmaceutically acceptable salt of claim 143, wherein Y² is a trivalent moiety connecting two L⁵ groups to L⁶, or connecting two L⁵ groups to R^{g}.

149. The compound or pharmaceutically acceptable salt of claim 148, wherein Y² is of the formula:

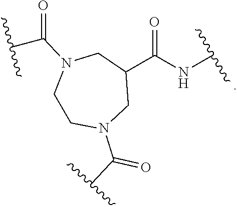 [SMILES: *NC(=O)C1CN(C(*)=O)CCN(C(*)=O)C1]

150. The compound or pharmaceutically acceptable salt of any one of claims 143-149, wherein L⁶ is a linker comprising 1-10 PEG units.

151. The compound or pharmaceutically acceptable salt of any one of claims 143-150, wherein L⁶ is of the formula:

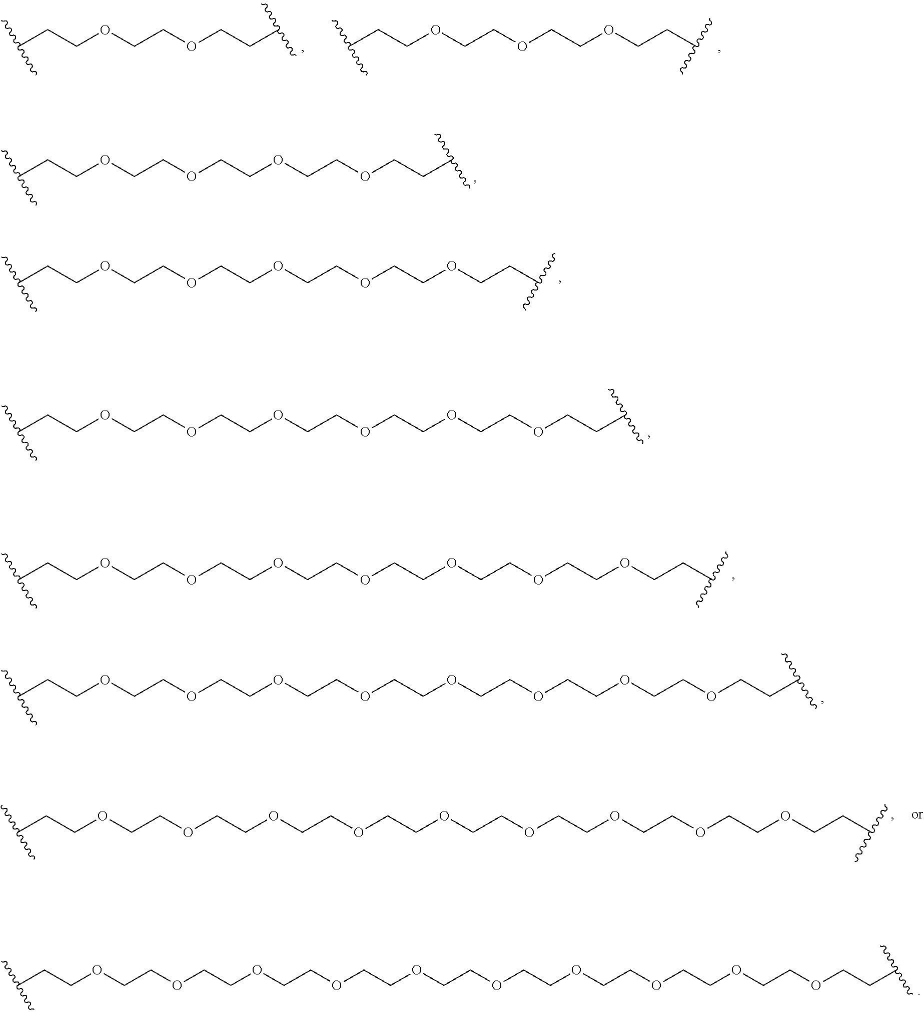

152. The compound or pharmaceutically acceptable salt of any one of claims 143-151, wherein L⁶ is of the formula:

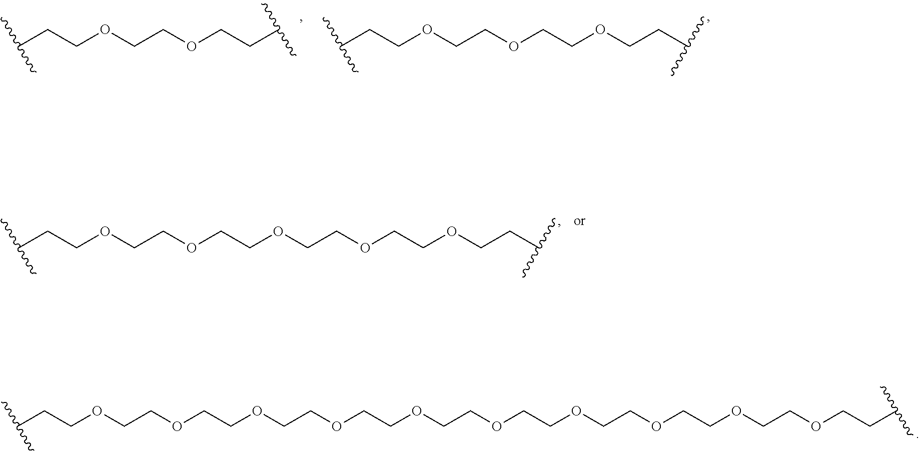 [SMILES: *CCOCCOCC* ; *CCOCCOCCOCC* ; *CCOCCOCCOCCOCCOCC* ; *CCOCCOCCOCCOCCOCCOCCOCCOCCOCCOCC*]

153. The compound or pharmaceutically acceptable salt of any one of claims 143-152, wherein z is 1 or 2, as valency permits.

154. The compound or pharmaceutically acceptable salt of any one of claims 143-153, wherein R^{g} is of the formula:

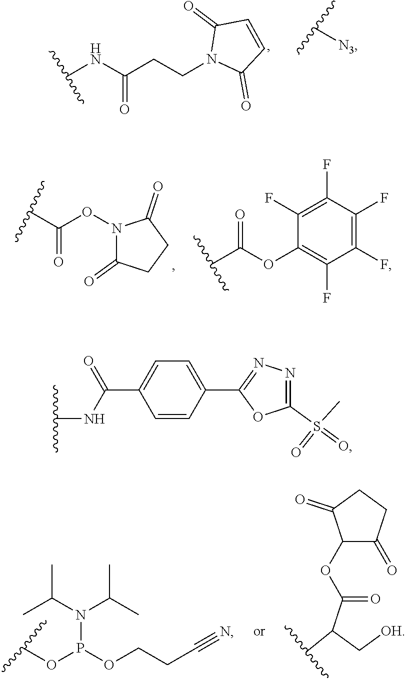

155. The compound or pharmaceutically acceptable salt of any one of claims 143-154, wherein at least one instance of L⁵ is independently a saturated lipid.

156. The compound or pharmaceutically acceptable salt of any one of claims 143-154, wherein at least one instance of L⁵ is independently an unsaturated lipid.

157. The compound or pharmaceutically acceptable salt of any one of claims 143-156, wherein at least one instance of L⁵ is independently a straight chain lipid.

158. The compound or pharmaceutically acceptable salt of any one of claims 143-157, wherein at least one instance of L⁵ is independently of the formula: 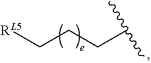
wherein
R^{L5} is H, or CO₂H; and
e is an integer between 5 and 35.

159. The compound or pharmaceutically acceptable salt of claim 158, wherein R^{L5} is H.

160. The compound or pharmaceutically acceptable salt of claim 158 or 159, wherein R^{L5} is CO₂H.

161. The compound or pharmaceutically acceptable salt of any one of claims 158-160, wherein e is 8, 9, 10, 11, 12, 13, 14, 15, 16, 17, 18, 19, or 20.

162. The compound or pharmaceutically acceptable salt of any one of claims 158-161, wherein e is 9, 11, 12, 13, 14, 15, or 17.

163. The compound or pharmaceutically acceptable salt of claim 158, wherein e is 13.

164. The compound or pharmaceutically acceptable salt of claim 163, wherein R^{L5} is H.

165. The compound or pharmaceutically acceptable salt of claim 158, wherein e is 12.

166. The compound or pharmaceutically acceptable salt of claim 165, wherein R^{L5} is CO₂H.

167. The compound or pharmaceutically acceptable salt of any one of claims 143-166, wherein at least one instance of L⁵ is independently a branched lipid

168. The compound or pharmaceutically acceptable salt of any one of claims 143-167, wherein at least one instance of L⁵ is independently of the formula:

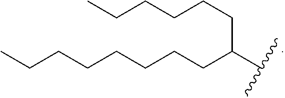 [SMILES: *C(CCCCCC)CCCCCCCC]

169. The compound or pharmaceutically acceptable salt of any one of claims 143-168, wherein at least one instance of L⁵ is independently of the formula:

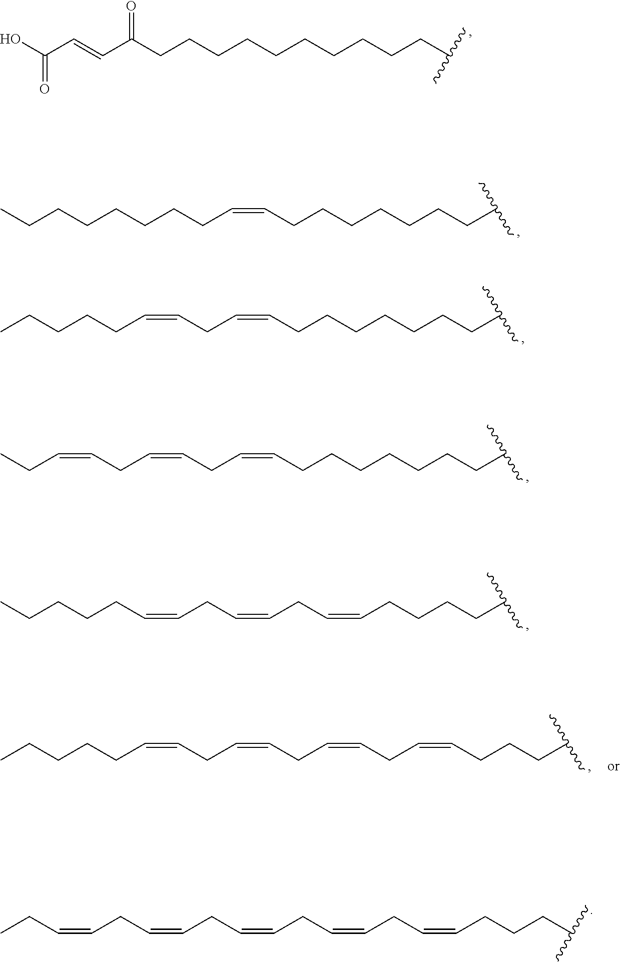

170. A compound or pharmaceutically acceptable salt thereof, of the formula: 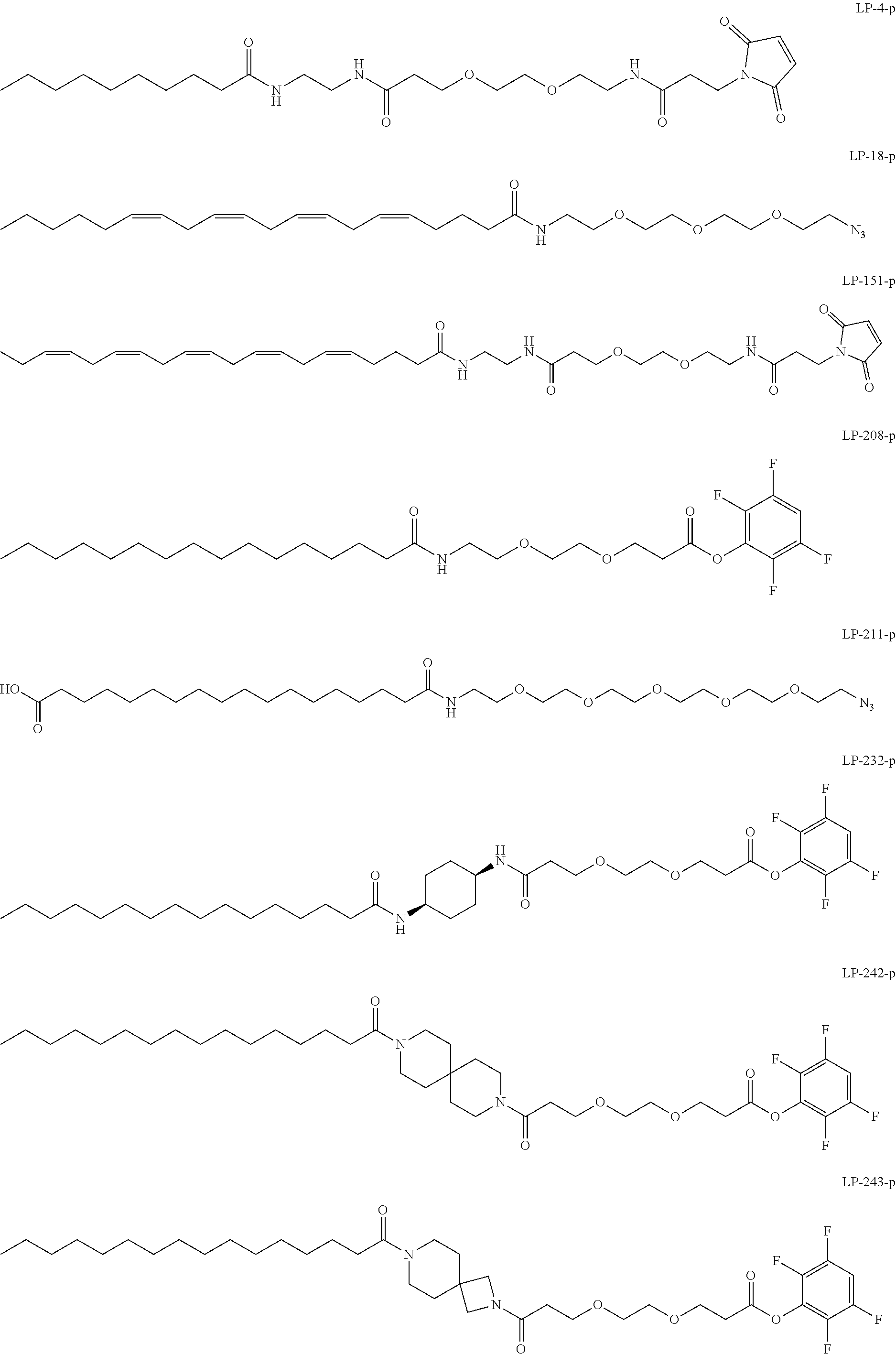 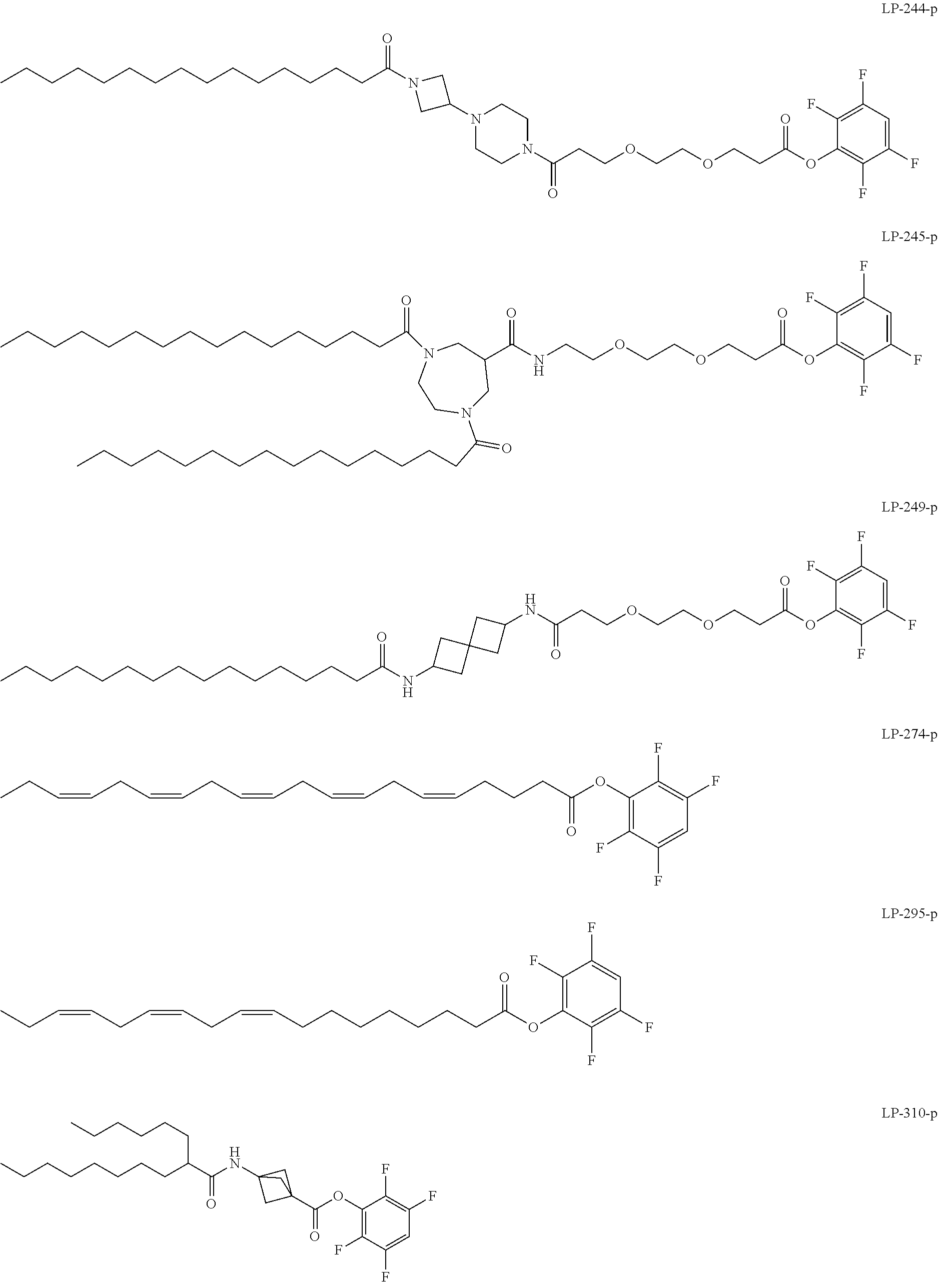 [SMILES: CCCCCCCCCCCCCCCC(=O)N1CC(N2CCN(C(=O)CCOCCOCCC(=O)Oc3c(F)c(F)cc(F)c3F)CC2)C1 ; CCCCCCCCCCCCCCCC(=O)N1CCN(C(=O)CCCCCCCCCCCCCCC)CC(C(=O)NCCOCCOCCC(=O)Oc2c(F)c(F)cc(F)c2F)C1 ; CCCCCCCCCCCCCCCC(=O)NC1CC2(C1)CC(NC(=O)CCOCCOCCC(=O)Oc1c(F)c(F)cc(F)c1F)C2 ; CC/C=C\C/C=C\C/C=C\C/C=C\C/C=C\CCCC(=O)Oc1c(F)c(F)cc(F)c1F ; CC/C=C\C/C=C\C/C=C\CCCCCCCC(=O)Oc1c(F)c(F)cc(F)c1F ; CCCCCCCCC(CCCCCC)C(=O)NC12CC(C(=O)Oc3c(F)c(F)cc(F)c3F)(C1)C2] 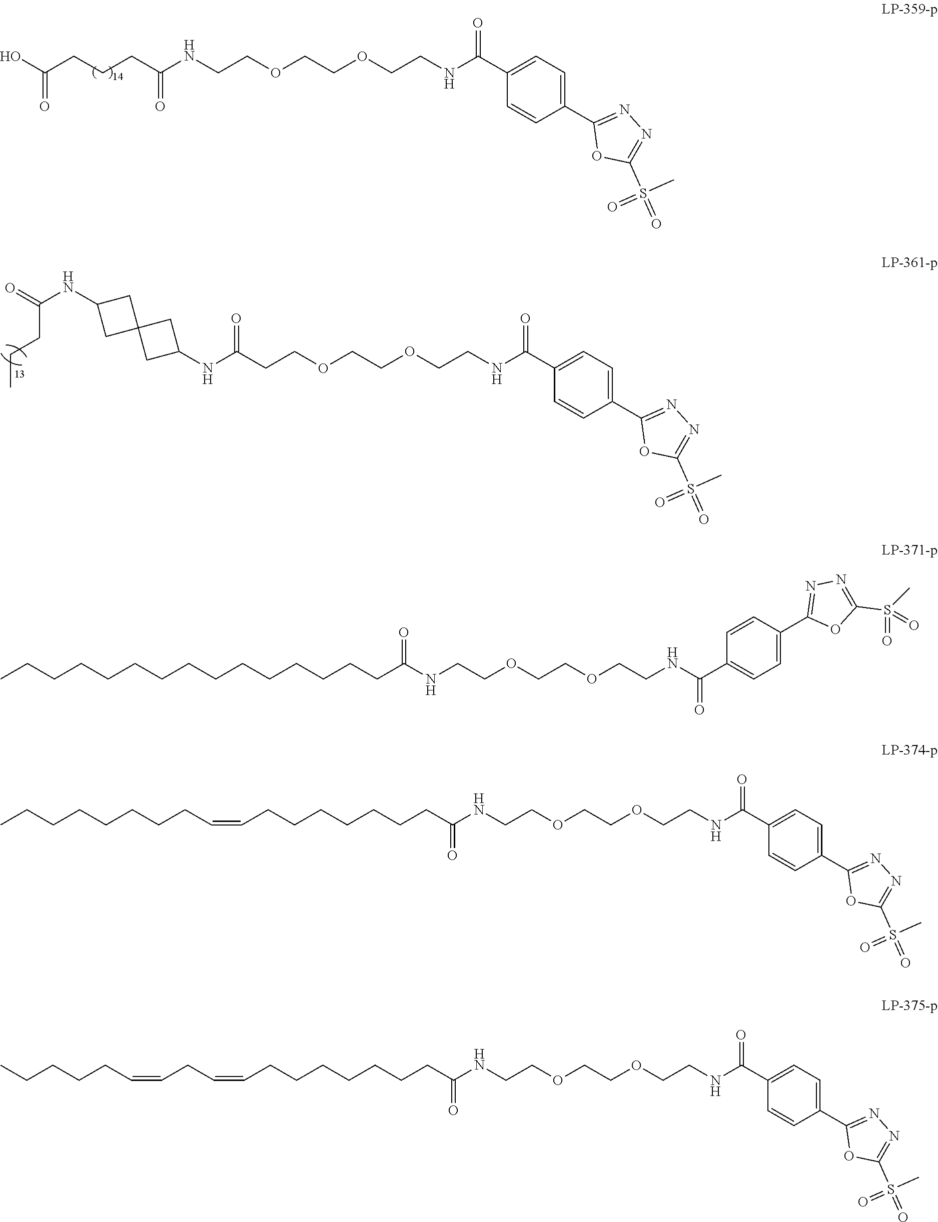 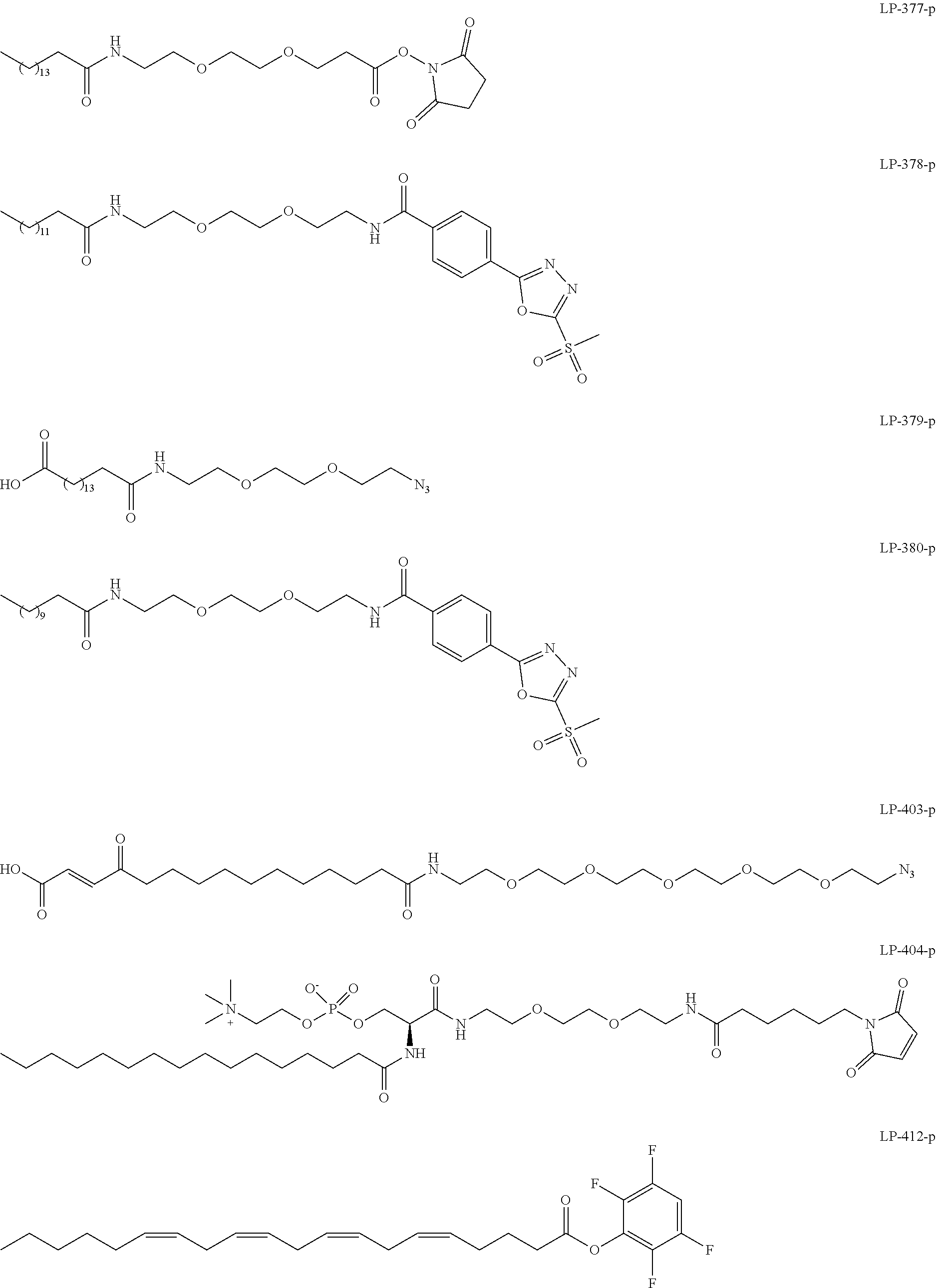 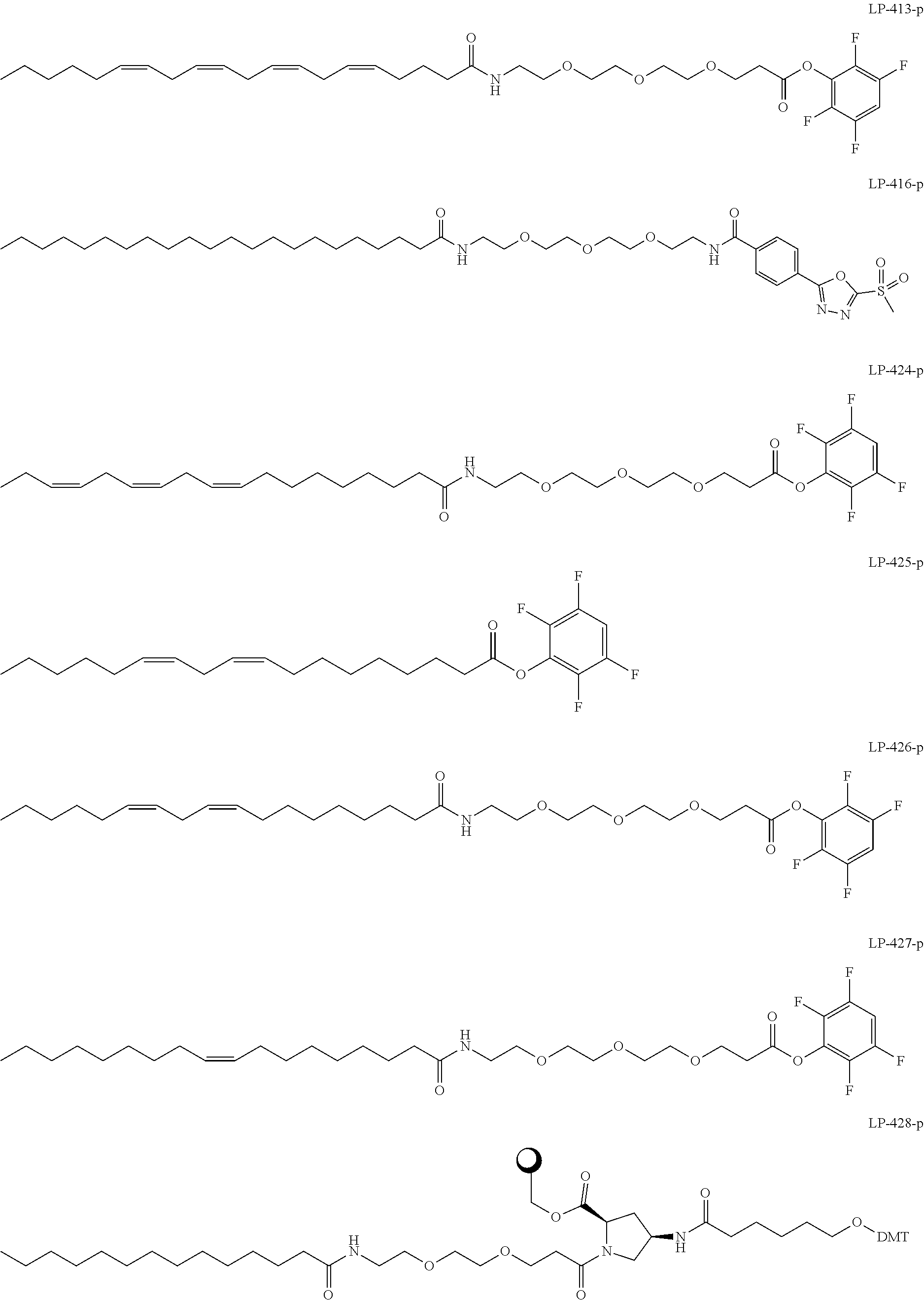 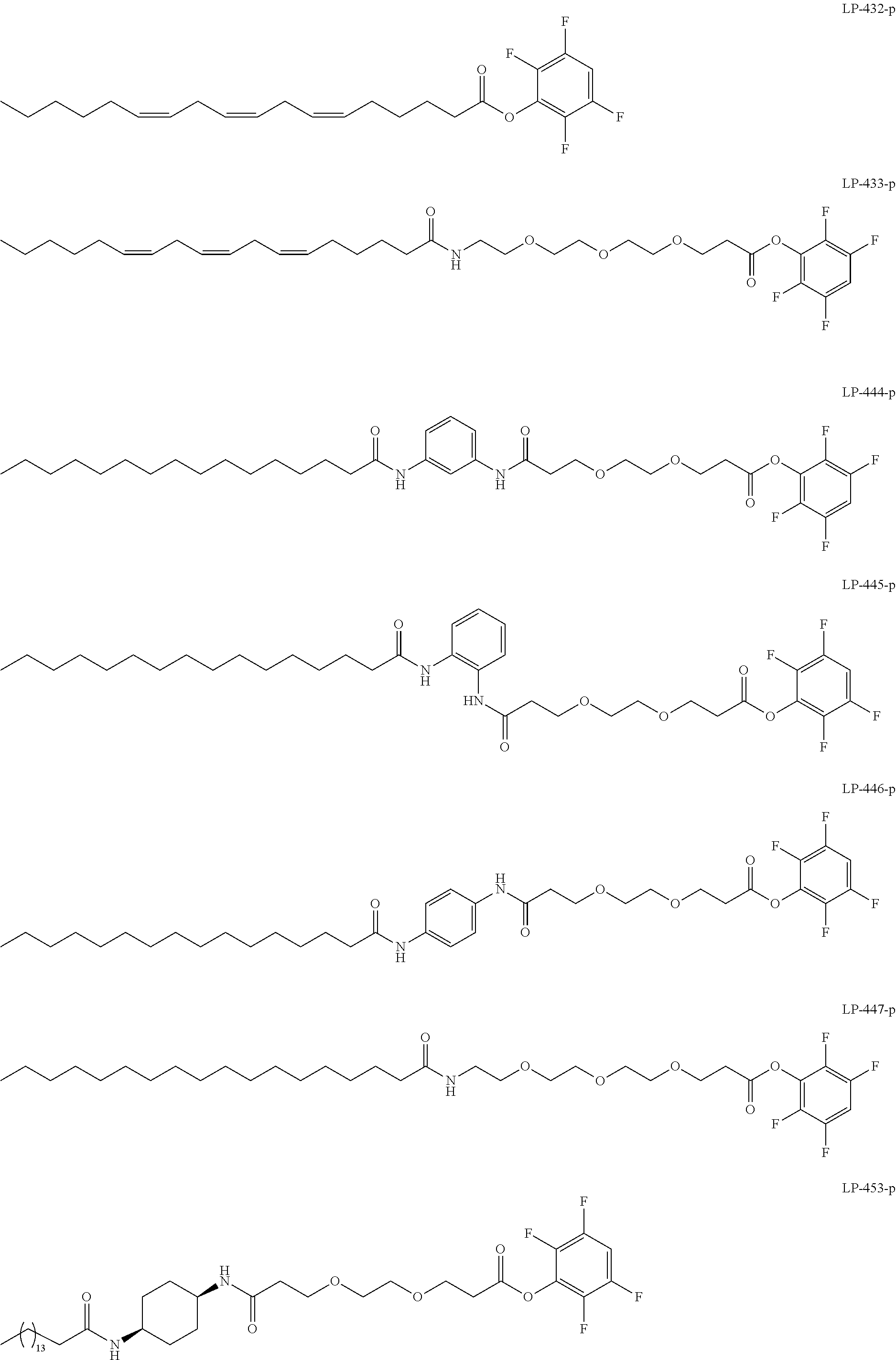 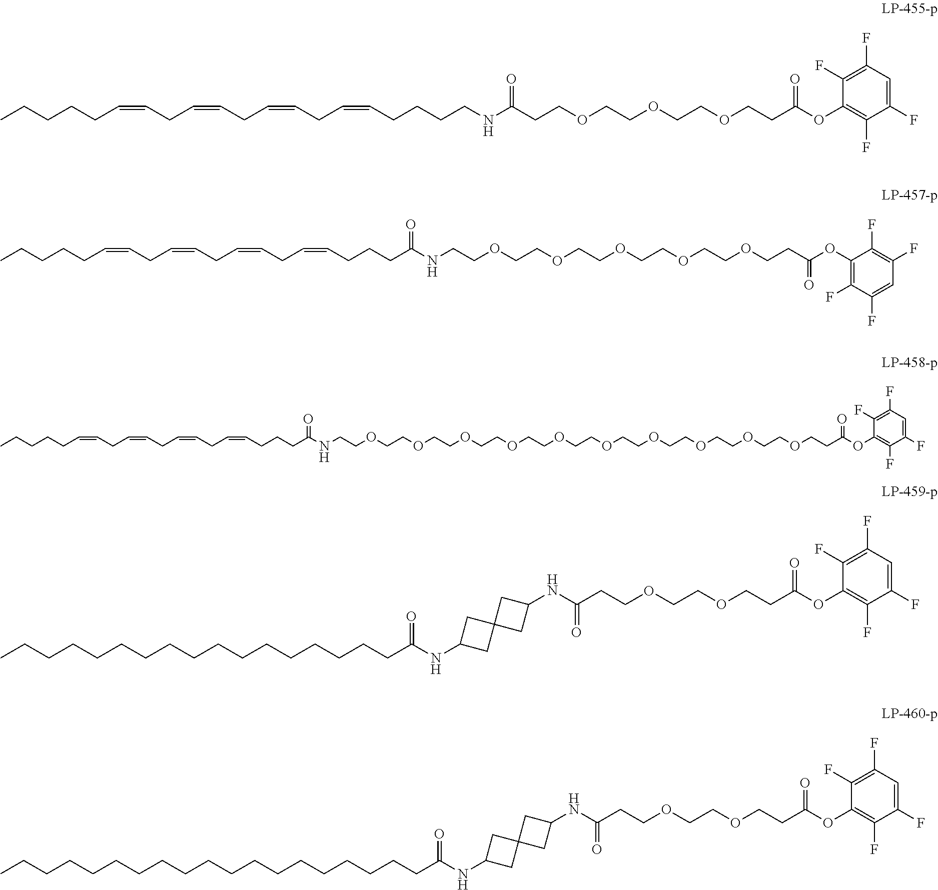 [SMILES: CCCCC/C=C\C/C=C\C/C=C\C/C=C\CCCCNC(=O)CCOCCOCCOCCC(=O)Oc1c(F)c(F)cc(F)c1F ; CCCCC/C=C\C/C=C\C/C=C\C/C=C\CCCC(=O)NCCOCCOCCOCCOCCOCCC(=O)Oc1c(F)c(F)cc(F)c1F ; CCCCC/C=C\C/C=C\C/C=C\C/C=C\CCCC(=O)NCCOCCOCCOCCOCCOCCOCCOCCOCCOCCOCCC(=O)Oc1c(F)c(F)cc(F)c1F ; CCCCCCCCCCCCCCCCCC(=O)NC1CC2(C1)CC(NC(=O)CCOCCOCCC(=O)Oc1c(F)c(F)cc(F)c1F)C2 ; CCCCCCCCCCCCCCCCCCCC(=O)NC1CC2(C1)CC(NC(=O)CCOCCOCCC(=O)Oc1c(F)c(F)cc(F)c1F)C2] 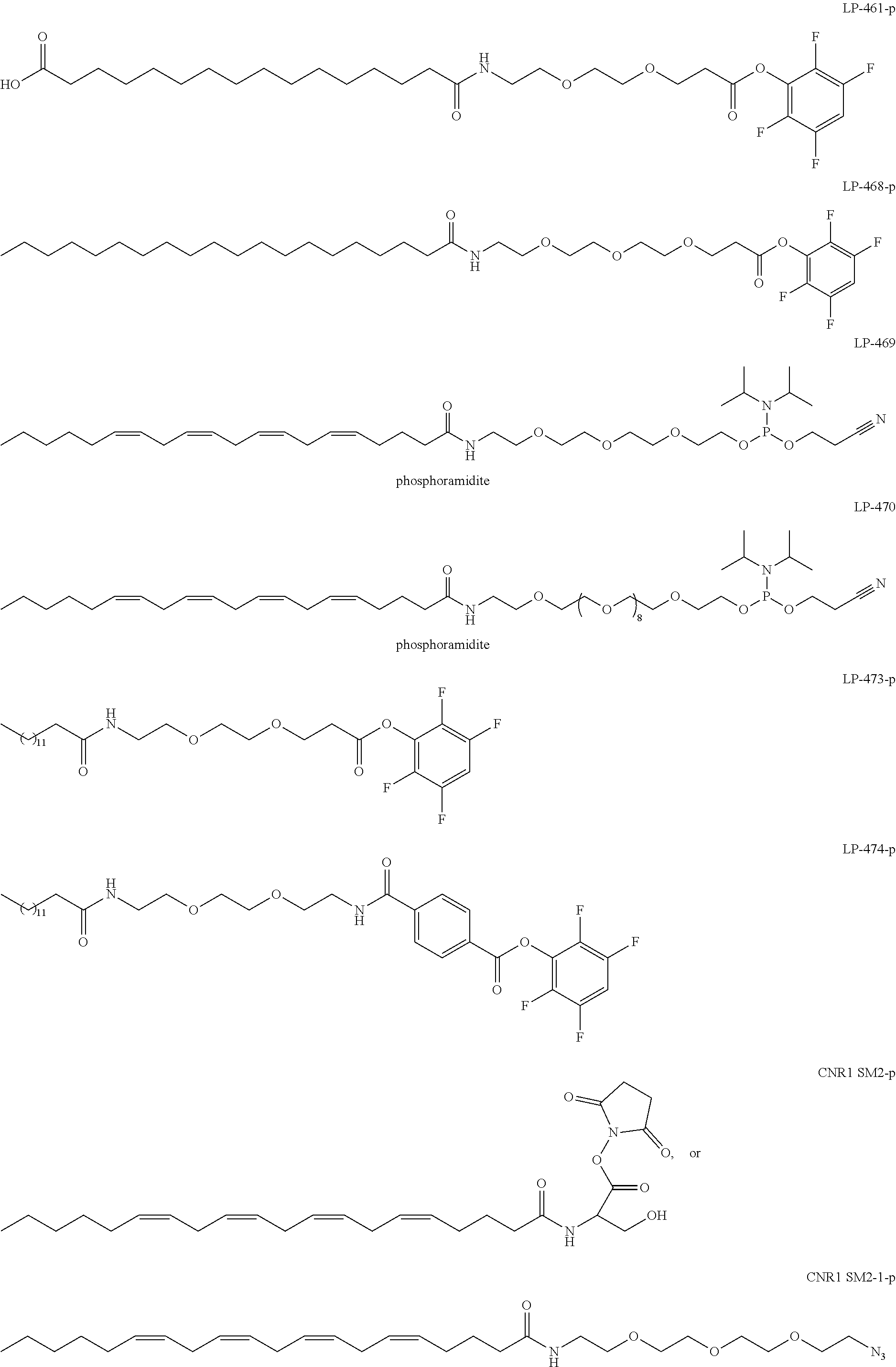
Wherein [symbol] indicates a solid support, such as a resin.

171. A method of synthesizing a lipid-oligonucleotide conjugate, comprising contacting an oligonucleotide with a compound of any one of compounds 143-170.


SEQUENCE LISTING (76 sequences)

SEQ ID NO: 1 - RNA, 21 nt, synthetic construct
  modified_base 1: mod_base = OTHER; note = 5'-cyclopropyl phosphonate-2'-O-methyladenosine
  modified_base 1^2: mod_base = OTHER; note = phosphorothioate linkage
  modified_base 2: mod_base = OTHER; note = 2'-fluoroguanosine
  modified_base 2^3: mod_base = OTHER; note = phosphorothioate linkage
  modified_base 3: mod_base = um
  modified_base 3^4: mod_base = OTHER; note = phosphorothioate linkage
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-O-methyladenosine
  modified_base 7: mod_base = um
  modified_base 8: mod_base = gm
  modified_base 9: mod_base = um
  modified_base 10: mod_base = OTHER; note = 2'-O-methyladenosine
  modified_base 11: mod_base = gm
  modified_base 12: mod_base = OTHER; note = 2'-fluoroadenosine
  modified_base 13: mod_base = gm
  modified_base 14: mod_base = OTHER; note = 2'-fluorouridine
  modified_base 15: mod_base = cm
  modified_base 16: mod_base = OTHER; note = 2'-fluoroguanosine
  modified_base 17: mod_base = um
  modified_base 18: mod_base = um
  modified_base 19: mod_base = gm
  modified_base 20: mod_base = OTHER; note = 2'-O-methyladenosine
  modified_base 20^21: mod_base = OTHER; note = phosphorothioate linkage
  modified_base 21: mod_base = cm
  source 1..21: mol_type = other RNA
agtaaatgtagagtcgttgac

SEQ ID NO: 2 - RNA, 21 nt, synthetic construct
  modified_base 1: mod_base = OTHER; note = 5'-cyclopropyl phosphonate-2'-O-methyluridine
  modified_base 1^2: mod_base = OTHER; note = phosphorothioate linkage
  modified_base 2: mod_base = OTHER; note = 2'-fluoroadenosine
  modified_base 2^3: mod_base = OTHER; note = phosphorothioate linkage
  modified_base 3: mod_base = um
  modified_base 4: mod_base = gm
  modified_base 5: mod_base = OTHER; note = 2'-O-methyladenosine
  modified_base 6: mod_base = um
  modified_base 7: mod_base = OTHER; note = 2'-fluoroadenosine
  modified_base 8: mod_base = gm
  modified_base 9: mod_base = OTHER; note = 2'-O-methyladenosine
  modified_base 10: mod_base = OTHER; note = 2'-O-methyladenosine
  modified_base 11: mod_base = um
  modified_base 12: mod_base = OTHER; note = 2'-fluorocytidine
  modified_base 13: mod_base = gm
  modified_base 14: mod_base = OTHER; note = 2'-fluorouridine
  modified_base 15: mod_base = gm
  modified_base 16: mod_base = OTHER; note = 2'-fluorocytidine
  modified_base 17: mod_base = um
  modified_base 18: mod_base = um
  modified_base 19: mod_base = cm
  modified_base 20: mod_base = OTHER; note = 2'-O-methyladenosine
  modified_base 20^21: mod_base = OTHER; note = phosphorothioate linkage
  modified_base 21: mod_base = cm
  source 1..21: mol_type = other RNA
tatgatagaatcgtgcttcac

SEQ ID NO: 3 - RNA, 21 nt, synthetic construct
  modified_base 1: mod_base = OTHER; note = 5'-cyclopropyl phosphonate-2'-O-methyluridine
  modified_base 1^2: mod_base = OTHER; note = phosphorothioate linkage
  modified_base 2: mod_base = OTHER; note = 2'-fluoroadenosine
  modified_base 2^3: mod_base = OTHER; note = phosphorothioate linkage
  modified_base 3: mod_base = gm
  modified_base 4: mod_base = gm
  modified_base 5: mod_base = OTHER; note = 2'-O-methyladenosine
  modified_base 6: mod_base = um
  modified_base 7: mod_base = OTHER; note = 2'-fluorouridine
  modified_base 8: mod_base = cm
  modified_base 9: mod_base = um
  modified_base 10: mod_base = gm
  modified_base 11: mod_base = gm
  modified_base 12: mod_base = OTHER; note = 2'-fluoroguanosine
  modified_base 13: mod_base = gm
  modified_base 14: mod_base = OTHER; note = 2'-fluorouridine
  modified_base 15: mod_base = OTHER; note = 2'-O-methyladenosine
  modified_base 16: mod_base = OTHER; note = 2'-fluorouridine
  modified_base 17: mod_base = gm
  modified_base 18: mod_base = um
  modified_base 19: mod_base = cm
  modified_base 20: mod_base = um
  modified_base 20^21: mod_base = OTHER; note = phosphorothioate linkage
  modified_base 21: mod_base = cm
  source 1..21: mol_type = other RNA
taggattctggggtatgtctc

SEQ ID NO: 4 - RNA, 21 nt, synthetic construct
  modified_base 1: mod_base = OTHER; note = 5'-cyclopropyl phosphonate-2'-O-methyluridine
  modified_base 1^2: mod_base = OTHER; note = phosphorothioate linkage
  modified_base 2: mod_base = OTHER; note = 2'-fluorocytidine
  modified_base 2^3: mod_base = OTHER; note = phosphorothioate linkage
  modified_base 3: mod_base = gm
  modified_base 4: mod_base = OTHER; note = 2'-O-methyladenosine
  modified_base 5: mod_base = OTHER; note = 2'-O-methyladenosine
  modified_base 6: mod_base = um
  modified_base 7: mod_base = OTHER; note = 2'-fluoroadenosine
  modified_base 8: mod_base = gm
  modified_base 9: mod_base = OTHER; note = 2'-O-methyladenosine
  modified_base 10: mod_base = um
  modified_base 11: mod_base = gm
  modified_base 12: mod_base = OTHER; note = 2'-fluorouridine
  modified_base 13: mod_base = cm
  modified_base 14: mod_base = OTHER; note = 2'-fluoroadenosine
  modified_base 15: mod_base = gm
  modified_base 16: mod_base = OTHER; note = 2'-fluorocytidine
  modified_base 17: mod_base = um
  modified_base 18: mod_base = cm
  modified_base 19: mod_base = gm
  modified_base 20: mod_base = cm
  modified_base 20^21: mod_base = OTHER; note = phosphorothioate linkage
  modified_base 21: mod_base = um
  source 1..21: mol_type = other RNA
tcgaatagatgtcagctcgct

SEQ ID NO: 5 - RNA, 21 nt, synthetic construct
  modified_base 1: mod_base = OTHER; note = 5'-cyclopropyl phosphonate-2'-O-methyluridine
  modified_base 1^2: mod_base = OTHER; note = phosphorothioate linkage
  modified_base 2: mod_base = OTHER; note = 2'-fluoroadenosine
  modified_base 2^3: mod_base = OTHER; note = phosphorothioate linkage
  modified_base 3: mod_base = um
  modified_base 4: mod_base = gm
  modified_base 5: mod_base = OTHER; note = 2'-O-methyladenosine
  modified_base 6: mod_base = um
  modified_base 7: mod_base = OTHER; note = 2'-O-methyladenosine
  modified_base 8: mod_base = gm
  modified_base 9: mod_base = OTHER; note = 2'-O-methyladenosine
  modified_base 10: mod_base = OTHER; note = 2'-O-methyladenosine
  modified_base 11: mod_base = um
  modified_base 12: mod_base = OTHER; note = 2'-fluorocytidine
  modified_base 13: mod_base = gm
  modified_base 14: mod_base = OTHER; note = 2'-fluorouridine
  modified_base 15: mod_base = gm
  modified_base 16: mod_base = OTHER; note = 2'-fluorocytidine
  modified_base 17: mod_base = um
  modified_base 18: mod_base = um
  modified_base 19: mod_base = cm
  modified_base 20: mod_base = OTHER; note = 2'-O-methyladenosine
  modified_base 20^21: mod_base = OTHER; note = phosphorothioate linkage
  modified_base 21: mod_base = cm
  source 1..21: mol_type = other RNA
tatgatagaatcgtgcttcac

SEQ ID NO: 6 - RNA, 21 nt, synthetic construct
  modified_base 1: mod_base = OTHER; note = 5'-cyclopropyl phosphonate-2'-O-methyluridine
  modified_base 1^2: mod_base = OTHER; note = phosphorothioate linkage
  modified_base 2: mod_base = OTHER; note = 2'-fluoroguanosine
  modified_base 2^3: mod_base = OTHER; note = phosphorothioate linkage
  modified_base 3: mod_base = um
  modified_base 4: mod_base = OTHER; note = 2'-O-methyladenosine
  modified_base 5: mod_base = um
  modified_base 6: mod_base = cm
  modified_base 7: mod_base = OTHER; note = 2'-fluoroadenosine
  modified_base 8: mod_base = um
  modified_base 9: mod_base = cm
  modified_base 10: mod_base = OTHER; note = 2'-O-methyladenosine
  modified_base 11: mod_base = OTHER; note = 2'-O-methyladenosine
  modified_base 12: mod_base = OTHER; note = 2'-fluoroguanosine
  modified_base 13: mod_base = cm
  modified_base 14: mod_base = OTHER; note = 2'-fluoroadenosine
  modified_base 15: mod_base = um
  modified_base 16: mod_base = OTHER; note = 2'-fluorouridine
  modified_base 17: mod_base = um
  modified_base 18: mod_base = cm
  modified_base 19: mod_base = OTHER; note = 2'-O-methyladenosine
  modified_base 20: mod_base = gm
  modified_base 20^21: mod_base = OTHER; note = phosphorothioate linkage
  modified_base 21: mod_base = gm
  source 1..21: mol_type = other RNA
tgtatcatcaagcatttcagg

SEQ ID NO: 7 - RNA, 21 nt, synthetic construct
  modified_base 1: mod_base = OTHER; note = 5'-cyclopropyl phosphonate-2'-O-methyluridine
  modified_base 1^2: mod_base = OTHER; note = phosphorothioate linkage
  modified_base 2: mod_base = OTHER; note = 2'-fluoroadenosine
  modified_base 2^3: mod_base = OTHER; note = phosphorothioate linkage
  modified_base 3: mod_base = um
  modified_base 4: mod_base = um
  modified_base 5: mod_base = gm
  modified_base 6: mod_base = um
  modified_base 7: mod_base = OTHER; note = 2'-fluoroadenosine
  modified_base 8: mod_base = um
  modified_base 9: mod_base = cm
  modified_base 10: mod_base = OTHER; note = 2'-O-methyladenosine
  modified_base 11: mod_base = um
  modified_base 12: mod_base = OTHER; note = 2'-fluorocytidine
  modified_base 13: mod_base = OTHER; note = 2'-O-methyladenosine
  modified_base 14: mod_base = OTHER; note = 2'-fluoroadenosine
  modified_base 15: mod_base = gm
  modified_base 16: mod_base = OTHER; note = 2'-fluorocytidine
  modified_base 17: mod_base = OTHER; note = 2'-O-methyladenosine
  modified_base 18: mod_base = um
  modified_base 19: mod_base = um
  modified_base 20: mod_base = um
  modified_base 20^21: mod_base = OTHER; note = phosphorothioate linkage
  modified_base 21: mod_base = cm
  source 1..21: mol_type = other RNA
tattgtatcatcaagcatttc

SEQ ID NO: 8 - RNA, 21 nt, synthetic construct
  modified_base 1: mod_base = OTHER; note = 5'-cyclopropyl phosphonate-2'-O-methyladenosine
  modified_base 1^2: mod_base = OTHER; note = phosphorothioate linkage
  modified_base 2: mod_base = OTHER; note = 2'-fluorouridine
  modified_base 2^3: mod_base = OTHER; note = phosphorothioate linkage
  modified_base 3: mod_base = um
  modified_base 4: mod_base = gm
  modified_base 5: mod_base = um
  modified_base 6: mod_base = um
  modified_base 7: mod_base = OTHER; note = 2'-fluoroguanosine
  modified_base 8: mod_base = gm
  modified_base 9: mod_base = OTHER; note = 2'-O-methyladenosine
  modified_base 10: mod_base = OTHER; note = 2'-O-methyladenosine
  modified_base 11: mod_base = cm
  modified_base 12: mod_base = OTHER; note = 2'-fluorouridine
  modified_base 13: mod_base = OTHER; note = 2'-O-methyladenosine
  modified_base 14: mod_base = OTHER; note = 2'-fluorouridine
  modified_base 15: mod_base = gm
  modified_base 16: mod_base = OTHER; note = 2'-fluoroadenosine
  modified_base 17: mod_base = cm
  modified_base 18: mod_base = OTHER; note = 2'-O-methyladenosine
  modified_base 19: mod_base = gm
  modified_base 20: mod_base = OTHER; note = 2'-O-methyladenosine
  modified_base 20^21: mod_base = OTHER; note = phosphorothioate linkage
  modified_base 21: mod_base = cm
  source 1..21: mol_type = other RNA
attgttggaactatgacagac

SEQ ID NO: 9 - RNA, 21 nt, synthetic construct
  modified_base 1: mod_base = OTHER; note = 5'-cyclopropyl phosphonate-2'-O-methyluridine
  modified_base 1^2: mod_base = OTHER; note = phosphorothioate linkage
  modified_base 2: mod_base = OTHER; note = 2'-fluorocytidine
  modified_base 2^3: mod_base = OTHER; note = phosphorothioate linkage
  modified_base 3: mod_base = OTHER; note = 2'-O-methyladenosine
  modified_base 4: mod_base = um
  modified_base 5: mod_base = gm
  modified_base 6: mod_base = OTHER; note = 2'-O-methyladenosine
  modified_base 7: mod_base = OTHER; note = 2'-fluorouridine
  modified_base 8: mod_base = OTHER; note = 2'-O-methyladenosine
  modified_base 9: mod_base = um
  modified_base 10: mod_base = um
  modified_base 11: mod_base = cm
  modified_base 12: mod_base = OTHER; note = 2'-fluoroadenosine
  modified_base 13: mod_base = gm
  modified_base 14: mod_base = OTHER; note = 2'-fluoroadenosine
  modified_base 15: mod_base = um
  modified_base 16: mod_base = OTHER; note = 2'-fluoroadenosine
  modified_base 17: mod_base = cm
  modified_base 18: mod_base = cm
  modified_base 19: mod_base = OTHER; note = 2'-O-methyladenosine
  modified_base 20: mod_base = gm
  modified_base 20^21: mod_base = OTHER; note = phosphorothioate linkage
  modified_base 21: mod_base = cm
  source 1..21: mol_type = other RNA
tcatgatattcagataccagc

SEQ ID NO: 10 - RNA, 21 nt, synthetic construct
  modified_base 1: mod_base = gm
  modified_base 2: mod_base = um
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = cm
  modified_base 7: mod_base = gm
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16: mod_base = um
  modified_base 17: mod_base = um
  modified_base 18: mod_base = um
  modified_base 19: mod_base = OTHER; note = 2'-O-methyladenosine
  modified_base 20: mod_base = cm
  modified_base 21: mod_base = um
  source 1..21: mol_type = other RNA
gtcaacgactctacatttact

SEQ ID NO: 11 - RNA, 21 nt, synthetic construct
  modified_base 1: mod_base = gm
  modified_base 2: mod_base = um
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = cm
  modified_base 7: mod_base = gm
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16: mod_base = um
  modified_base 17: mod_base = um
  modified_base 18: mod_base = um
  modified_base 19: mod_base = OTHER; note = 2'-O-methyladenosine
  modified_base 20: mod_base = cm
  modified_base 21: mod_base = um
  source 1..21: mol_type = other RNA
gtcaacgactctacatttact

SEQ ID NO: 12 - RNA, 21 nt, synthetic construct
  modified_base 1: mod_base = gm
  modified_base 2: mod_base = um
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = cm
  modified_base 7: mod_base = gm
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16: mod_base = um
  modified_base 17: mod_base = um
  modified_base 18: mod_base = um
  modified_base 19: mod_base = OTHER; note = 2'-O-methyladenosine
  modified_base 20: mod_base = cm
  modified_base 21: mod_base = um
  source 1..21: mol_type = other RNA
gtcaacgactctacatttact

SEQ ID NO: 13 - RNA, 21 nt, synthetic construct
  modified_base 1: mod_base = gm
  modified_base 2: mod_base = um
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = cm
  modified_base 7: mod_base = gm
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16: mod_base = um
  modified_base 17: mod_base = um
  modified_base 18: mod_base = um
  modified_base 19: mod_base = OTHER; note = 2'-O-methyladenosine
  modified_base 20: mod_base = cm
  modified_base 21: mod_base = um
  source 1..21: mol_type = other RNA
gtcaacgactctacatttact

SEQ ID NO: 14 - RNA, 21 nt, synthetic construct
  modified_base 1: mod_base = gm
  modified_base 2: mod_base = um
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = cm
  modified_base 7: mod_base = gm
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16: mod_base = um
  modified_base 17: mod_base = um
  modified_base 18: mod_base = um
  modified_base 19: mod_base = OTHER; note = 2'-O-methyladenosine
  modified_base 20: mod_base = cm
  modified_base 21: mod_base = um
  source 1..21: mol_type = other RNA
gtcaacgactctacatttact

SEQ ID NO: 15 - RNA, 21 nt, synthetic construct
  modified_base 1: mod_base = gm
  modified_base 2: mod_base = um
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = cm
  modified_base 7: mod_base = gm
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16: mod_base = um
  modified_base 17: mod_base = um
  modified_base 18: mod_base = um
  modified_base 19: mod_base = OTHER; note = 2'-O-methyladenosine
  modified_base 20: mod_base = cm
  modified_base 21: mod_base = um
  source 1..21: mol_type = other RNA
gtcaacgactctacatttact

SEQ ID NO: 16 - RNA, 21 nt, synthetic construct
  modified_base 1: mod_base = gm
  modified_base 2: mod_base = um
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = cm
  modified_base 7: mod_base = gm
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16: mod_base = um
  modified_base 17: mod_base = um
  modified_base 18: mod_base = um
  modified_base 19: mod_base = OTHER; note = 2'-O-methyladenosine
  modified_base 20: mod_base = cm
  modified_base 21: mod_base = um
  source 1..21: mol_type = other RNA
gtcaacgactctacatttact

SEQ ID NO: 17 - RNA, 21 nt, synthetic construct
  modified_base 1: mod_base = gm
  modified_base 2: mod_base = um
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = cm
  modified_base 7: mod_base = gm
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16: mod_base = um
  modified_base 17: mod_base = um
  modified_base 18: mod_base = um
  modified_base 19: mod_base = OTHER; note = 2'-O-methyladenosine
  modified_base 20: mod_base = cm
  modified_base 21: mod_base = um
  source 1..21: mol_type = other RNA
gtcaacgactctacatttact

SEQ ID NO: 18 - RNA, 21 nt, synthetic construct
  modified_base 1: mod_base = gm
  modified_base 2: mod_base = um
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = cm
  modified_base 7: mod_base = gm
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16: mod_base = um
  modified_base 17: mod_base = um
  modified_base 18: mod_base = um
  modified_base 19: mod_base = OTHER; note = 2'-O-methyladenosine
  modified_base 20: mod_base = cm
  modified_base 21: mod_base = um
  source 1..21: mol_type = other RNA
gtcaacgactctacatttact

SEQ ID NO: 19 - RNA, 21 nt, synthetic construct
  modified_base 1: mod_base = gm
  modified_base 2: mod_base = um
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = cm
  modified_base 7: mod_base = gm
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16: mod_base = um
  modified_base 17: mod_base = um
  modified_base 18: mod_base = um
  modified_base 19: mod_base = OTHER; note = 2'-O-methyladenosine
  modified_base 20: mod_base = cm
  modified_base 21: mod_base = um
  source 1..21: mol_type = other RNA
gtcaacgactctacatttact

SEQ ID NO: 20 - RNA, 21 nt, synthetic construct
  modified_base 1: mod_base = gm
  modified_base 2: mod_base = um
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = cm
  modified_base 7: mod_base = gm
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16: mod_base = um
  modified_base 17: mod_base = um
  modified_base 18: mod_base = um
  modified_base 19: mod_base = OTHER; note = 2'-O-methyladenosine
  modified_base 20: mod_base = cm
  modified_base 21: mod_base = um
  source 1..21: mol_type = other RNA
gtcaacgactctacatttact

SEQ ID NO: 21 - RNA, 21 nt, synthetic construct
  modified_base 1: mod_base = gm
  modified_base 2: mod_base = um
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = cm
  modified_base 7: mod_base = gm
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16: mod_base = um
  modified_base 17: mod_base = um
  modified_base 18: mod_base = um
  modified_base 19: mod_base = OTHER; note = 2'-O-methyladenosine
  modified_base 20: mod_base = cm
  modified_base 21: mod_base = um
  source 1..21: mol_type = other RNA
gtcaacgactctacatttact

SEQ ID NO: 22 - RNA, 21 nt, synthetic construct
  modified_base 1: mod_base = gm
  modified_base 2: mod_base = um
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = cm
  modified_base 7: mod_base = gm
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16: mod_base = um
  modified_base 17: mod_base = um
  modified_base 18: mod_base = um
  modified_base 19: mod_base = OTHER; note = 2'-O-methyladenosine
  modified_base 20: mod_base = cm
  modified_base 21: mod_base = um
  source 1..21: mol_type = other RNA
gtcaacgactctacatttact

SEQ ID NO: 23 - RNA, 21 nt, synthetic construct
  modified_base 1: mod_base = gm
  modified_base 2: mod_base = um
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = cm
  modified_base 7: mod_base = gm
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16: mod_base = um
  modified_base 17: mod_base = um
  modified_base 18: mod_base = um
  modified_base 19: mod_base = OTHER; note = 2'-O-methyladenosine
  modified_base 20: mod_base = cm
  modified_base 21: mod_base = um
  source 1..21: mol_type = other RNA
gtcaacgactctacatttact

SEQ ID NO: 24 - RNA, 21 nt, synthetic construct
  modified_base 1: mod_base = gm
  modified_base 2: mod_base = um
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = cm
  modified_base 7: mod_base = gm
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16: mod_base = um
  modified_base 17: mod_base = um
  modified_base 18: mod_base = um
  modified_base 19: mod_base = OTHER; note = 2'-O-methyladenosine
  modified_base 20: mod_base = cm
  modified_base 21: mod_base = um
  source 1..21: mol_type = other RNA
gtcaacgactctacatttact

SEQ ID NO: 25 - RNA, 21 nt, synthetic construct
  modified_base 1: mod_base = gm
  modified_base 2: mod_base = um
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = cm
  modified_base 7: mod_base = gm
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16: mod_base = um
  modified_base 17: mod_base = um
  modified_base 18: mod_base = um
  modified_base 19: mod_base = OTHER; note = 2'-O-methyladenosine
  modified_base 20: mod_base = cm
  modified_base 21: mod_base = um
  source 1..21: mol_type = other RNA
gtcaacgactctacatttact

SEQ ID NO: 26 - RNA, 21 nt, synthetic construct
  modified_base 1: mod_base = gm
  modified_base 2: mod_base = um
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = cm
  modified_base 7: mod_base = gm
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16: mod_base = um
  modified_base 17: mod_base = um
  modified_base 18: mod_base = um
  modified_base 19: mod_base = OTHER; note = 2'-O-methyladenosine
  modified_base 20: mod_base = cm
  modified_base 21: mod_base = um
  source 1..21: mol_type = other RNA
gtcaacgactctacatttact

SEQ ID NO: 27 - RNA, 21 nt, synthetic construct
  modified_base 1: mod_base = gm
  modified_base 2: mod_base = um
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = cm
  modified_base 7: mod_base = gm
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16: mod_base = um
  modified_base 17: mod_base = um
  modified_base 18: mod_base = um
  modified_base 19: mod_base = OTHER; note = 2'-O-methyladenosine
  modified_base 20: mod_base = cm
  modified_base 21: mod_base = um
  source 1..21: mol_type = other RNA
gtcaacgactctacatttact

SEQ ID NO: 28 - RNA, 21 nt, synthetic construct
  modified_base 1: mod_base = gm
  modified_base 2: mod_base = um
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = cm
  modified_base 7: mod_base = gm
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16: mod_base = um
  modified_base 17: mod_base = um
  modified_base 18: mod_base = um
  modified_base 19: mod_base = OTHER; note = 2'-O-methyladenosine
  modified_base 20: mod_base = cm
  modified_base 21: mod_base = um
  source 1..21: mol_type = other RNA
gtcaacgactctacatttact

SEQ ID NO: 29 - RNA, 21 nt, synthetic construct
  modified_base 1: mod_base = gm
  modified_base 2: mod_base = um
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = cm
  modified_base 7: mod_base = gm
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16: mod_base = um
  modified_base 17: mod_base = um
  modified_base 18: mod_base = um
  modified_base 19: mod_base = OTHER; note = 2'-O-methyladenosine
  modified_base 20: mod_base = cm
  modified_base 21: mod_base = um
  source 1..21: mol_type = other RNA
gtcaacgactctacatttact

SEQ ID NO: 30 - RNA, 21 nt, synthetic construct
  modified_base 1: mod_base = gm
  modified_base 2: mod_base = um
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = cm
  modified_base 7: mod_base = gm
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16: mod_base = um
  modified_base 17: mod_base = um
  modified_base 18: mod_base = um
  modified_base 19: mod_base = OTHER; note = 2'-O-methyladenosine
  modified_base 20: mod_base = cm
  modified_base 21: mod_base = um
  source 1..21: mol_type = other RNA
gtcaacgactctacatttact

SEQ ID NO: 31 - RNA, 21 nt, synthetic construct
  modified_base 1: mod_base = gm
  modified_base 2: mod_base = um
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = cm
  modified_base 7: mod_base = gm
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16: mod_base = um
  modified_base 17: mod_base = um
  modified_base 18: mod_base = um
  modified_base 19: mod_base = OTHER; note = 2'-O-methyladenosine
  modified_base 20: mod_base = cm
  modified_base 21: mod_base = um
  source 1..21: mol_type = other RNA
gtcaacgactctacatttact

SEQ ID NO: 32 - RNA, 21 nt, synthetic construct
  modified_base 1: mod_base = gm
  modified_base 2: mod_base = um
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = cm
  modified_base 7: mod_base = gm
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16: mod_base = um
  modified_base 17: mod_base = um
  modified_base 18: mod_base = um
  modified_base 19: mod_base = OTHER; note = 2'-O-methyladenosine
  modified_base 20: mod_base = cm
  modified_base 21: mod_base = um
  source 1..21: mol_type = other RNA
gtcaacgactctacatttact

SEQ ID NO: 33 - RNA, 21 nt, synthetic construct
  modified_base 1: mod_base = gm
  modified_base 2: mod_base = um
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = cm
  modified_base 7: mod_base = gm
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16: mod_base = um
  modified_base 17: mod_base = um
  modified_base 18: mod_base = um
  modified_base 19: mod_base = OTHER; note = 2'-O-methyladenosine
  modified_base 20: mod_base = cm
  modified_base 21: mod_base = um
  source 1..21: mol_type = other RNA
gtcaacgactctacatttact

SEQ ID NO: 34 - RNA, 21 nt, synthetic construct
  modified_base 1: mod_base = gm
  modified_base 2: mod_base = um
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = cm
  modified_base 7: mod_base = gm
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16: mod_base = um
  modified_base 17: mod_base = um
  modified_base 18: mod_base = um
  modified_base 19: mod_base = OTHER; note = 2'-O-methyladenosine
  modified_base 20: mod_base = cm
  modified_base 21: mod_base = um
  source 1..21: mol_type = other RNA
gtcaacgactctacatttact

SEQ ID NO: 35 - RNA, 21 nt, synthetic construct
  modified_base 1: mod_base = gm
  modified_base 2: mod_base = um
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = cm
  modified_base 7: mod_base = gm
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16: mod_base = um
  modified_base 17: mod_base = um
  modified_base 18: mod_base = um
  modified_base 19: mod_base = OTHER; note = 2'-O-methyladenosine
  modified_base 20: mod_base = cm
  modified_base 21: mod_base = um
  source 1..21: mol_type = other RNA
gtcaacgactctacatttact

SEQ ID NO: 36 - RNA, 21 nt, synthetic construct
  modified_base 1: mod_base = gm
  modified_base 2: mod_base = um
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = cm
  modified_base 7: mod_base = gm
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16: mod_base = um
  modified_base 17: mod_base = um
  modified_base 18: mod_base = um
  modified_base 19: mod_base = OTHER; note = 2'-O-methyladenosine
  modified_base 20: mod_base = cm
  modified_base 21: mod_base = um
  source 1..21: mol_type = other RNA
gtcaacgactctacatttact

SEQ ID NO: 37 - RNA, 21 nt, synthetic construct
  modified_base 1: mod_base = gm
  modified_base 2: mod_base = um
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = cm
  modified_base 7: mod_base = gm
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16: mod_base = um
  modified_base 17: mod_base = um
  modified_base 18: mod_base = um
  modified_base 19: mod_base = OTHER; note = 2'-O-methyladenosine
  modified_base 20: mod_base = cm
  modified_base 21: mod_base = um
  source 1..21: mol_type = other RNA
gtcaacgactctacatttact

SEQ ID NO: 38 - RNA, 21 nt, synthetic construct
  modified_base 1: mod_base = gm
  modified_base 2: mod_base = um
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = cm
  modified_base 7: mod_base = gm
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16: mod_base = um
  modified_base 17: mod_base = um
  modified_base 18: mod_base = um
  modified_base 19: mod_base = OTHER; note = 2'-O-methyladenosine
  modified_base 20: mod_base = cm
  modified_base 21: mod_base = um
  source 1..21: mol_type = other RNA
gtcaacgactctacatttact

SEQ ID NO: 39 - RNA, 21 nt, synthetic construct
  modified_base 1: mod_base = gm
  modified_base 2: mod_base = um
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = cm
  modified_base 7: mod_base = gm
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16: mod_base = um
  modified_base 17: mod_base = um
  modified_base 18: mod_base = um
  modified_base 19: mod_base = OTHER; note = 2'-O-methyladenosine
  modified_base 20: mod_base = cm
  modified_base 21: mod_base = um
  source 1..21: mol_type = other RNA
gtcaacgactctacatttact

SEQ ID NO: 40 - RNA, 21 nt, synthetic construct
  modified_base 1: mod_base = gm
  modified_base 2: mod_base = um
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = cm
  modified_base 7: mod_base = gm
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16: mod_base = um
  modified_base 17: mod_base = um
  modified_base 18: mod_base = um
  modified_base 19: mod_base = OTHER; note = 2'-O-methyladenosine
  modified_base 20: mod_base = cm
  modified_base 21: mod_base = um
  source 1..21: mol_type = other RNA
gtcaacgactctacatttact

SEQ ID NO: 41 - RNA, 21 nt, synthetic construct
  modified_base 1: mod_base = gm
  modified_base 2: mod_base = um
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = cm
  modified_base 7: mod_base = gm
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16: mod_base = um
  modified_base 17: mod_base = um
  modified_base 18: mod_base = um
  modified_base 19: mod_base = OTHER; note = 2'-O-methyladenosine
  modified_base 20: mod_base = cm
  modified_base 21: mod_base = um
  source 1..21: mol_type = other RNA
gtcaacgactctacatttact

SEQ ID NO: 42 - RNA, 21 nt, synthetic construct
  modified_base 1: mod_base = gm
  modified_base 2: mod_base = um
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = cm
  modified_base 7: mod_base = gm
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16: mod_base = um
  modified_base 17: mod_base = um
  modified_base 18: mod_base = um
  modified_base 19: mod_base = OTHER; note = 2'-O-methyladenosine
  modified_base 20: mod_base = cm
  modified_base 21: mod_base = um
  source 1..21: mol_type = other RNA
gtcaacgactctacatttact

SEQ ID NO: 43 - RNA, 21 nt, synthetic construct
  modified_base 1: mod_base = gm
  modified_base 2: mod_base = um
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = cm
  modified_base 7: mod_base = gm
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16: mod_base = um
  modified_base 17: mod_base = um
  modified_base 18: mod_base = um
  modified_base 19: mod_base = OTHER; note = 2'-O-methyladenosine
  modified_base 20: mod_base = cm
  modified_base 21: mod_base = um
  source 1..21: mol_type = other RNA
gtcaacgactctacatttact

SEQ ID NO: 44 - RNA, 21 nt, synthetic construct
  modified_base 1: mod_base = gm
  modified_base 2: mod_base = um
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = cm
  modified_base 7: mod_base = gm
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16: mod_base = um
  modified_base 17: mod_base = um
  modified_base 18: mod_base = um
  modified_base 19: mod_base = OTHER; note = 2'-O-methyladenosine
  modified_base 20: mod_base = cm
  modified_base 21: mod_base = um
  source 1..21: mol_type = other RNA
gtcaacgactctacatttact

SEQ ID NO: 45 - RNA, 21 nt, synthetic construct
  modified_base 1: mod_base = gm
  modified_base 2: mod_base = um
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = cm
  modified_base 7: mod_base = gm
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16: mod_base = um
  modified_base 17: mod_base = um
  modified_base 18: mod_base = um
  modified_base 19: mod_base = OTHER; note = 2'-O-methyladenosine
  modified_base 20: mod_base = cm
  modified_base 21: mod_base = um
  source 1..21: mol_type = other RNA
gtcaacgactctacatttact

SEQ ID NO: 46 - RNA, 21 nt, synthetic construct
  modified_base 1: mod_base = gm
  modified_base 2: mod_base = um
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = cm
  modified_base 7: mod_base = gm
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16: mod_base = um
  modified_base 17: mod_base = um
  modified_base 18: mod_base = um
  modified_base 19: mod_base = OTHER; note = 2'-O-methyladenosine
  modified_base 20: mod_base = cm
  modified_base 21: mod_base = um
  source 1..21: mol_type = other RNA
gtcaacgactctacatttact

SEQ ID NO: 47 - RNA, 21 nt, synthetic construct
  modified_base 1: mod_base = gm
  modified_base 2: mod_base = um
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = cm
  modified_base 7: mod_base = gm
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16: mod_base = um
  modified_base 17: mod_base = um
  modified_base 18: mod_base = um
  modified_base 19: mod_base = OTHER; note = 2'-O-methyladenosine
  modified_base 20: mod_base = cm
  modified_base 21: mod_base = um
  source 1..21: mol_type = other RNA
gtcaacgactctacatttact

SEQ ID NO: 48 - RNA, 21 nt, synthetic construct
  modified_base 1: mod_base = gm
  modified_base 2: mod_base = um
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = cm
  modified_base 7: mod_base = gm
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16: mod_base = um
  modified_base 17: mod_base = um
  modified_base 18: mod_base = um
  modified_base 19: mod_base = OTHER; note = 2'-O-methyladenosine
  modified_base 20: mod_base = cm
  modified_base 21: mod_base = um
  source 1..21: mol_type = other RNA
gtcaacgactctacatttact

SEQ ID NO: 49 - RNA, 21 nt, synthetic construct
  modified_base 1: mod_base = gm
  modified_base 2: mod_base = um
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = cm
  modified_base 7: mod_base = gm
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16: mod_base = um
  modified_base 17: mod_base = um
  modified_base 18: mod_base = um
  modified_base 19: mod_base = OTHER; note = 2'-O-methyladenosine
  modified_base 20: mod_base = cm
  modified_base 21: mod_base = um
  source 1..21: mol_type = other RNA
gtcaacgactctacatttact

SEQ ID NO: 50 - RNA, 21 nt, synthetic construct
  modified_base 1: mod_base = gm
  modified_base 2: mod_base = um
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = cm
  modified_base 7: mod_base = gm
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16: mod_base = um
  modified_base 17: mod_base = um
  modified_base 18: mod_base = um
  modified_base 19: mod_base = OTHER; note = 2'-O-methyladenosine
  modified_base 20: mod_base = cm
  modified_base 21: mod_base = um
  source 1..21: mol_type = other RNA
gtcaacgactctacatttact

SEQ ID NO: 51 - RNA, 21 nt, synthetic construct
  modified_base 1: mod_base = gm
  modified_base 2: mod_base = um
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = cm
  modified_base 7: mod_base = gm
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16: mod_base = um
  modified_base 17: mod_base = um
  modified_base 18: mod_base = um
  modified_base 19: mod_base = OTHER; note = 2'-O-methyladenosine
  modified_base 20: mod_base = cm
  modified_base 21: mod_base = um
  source 1..21: mol_type = other RNA
gtcaacgactctacatttact

SEQ ID NO: 52 - RNA, 21 nt, synthetic construct
  modified_base 1: mod_base = gm
  modified_base 2: mod_base = um
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = cm
  modified_base 7: mod_base = gm
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16: mod_base = um
  modified_base 17: mod_base = um
  modified_base 18: mod_base = um
  modified_base 19: mod_base = OTHER; note = 2'-O-methyladenosine
  modified_base 20: mod_base = cm
  modified_base 21: mod_base = um
  source 1..21: mol_type = other RNA
gtcaacgactctacatttact

SEQ ID NO: 53 - RNA, 21 nt, synthetic construct
  modified_base 1: mod_base = gm
  modified_base 2: mod_base = um
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = cm
  modified_base 7: mod_base = gm
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16: mod_base = um
  modified_base 17: mod_base = um
  modified_base 18: mod_base = um
  modified_base 19: mod_base = OTHER; note = 2'-O-methyladenosine
  modified_base 20: mod_base = cm
  modified_base 21: mod_base = um
  source 1..21: mol_type = other RNA
gtcaacgactctacatttact

SEQ ID NO: 54 - RNA, 21 nt, synthetic construct
  modified_base 1: mod_base = gm
  modified_base 2: mod_base = um
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = cm
  modified_base 7: mod_base = gm
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16: mod_base = um
  modified_base 17: mod_base = um
  modified_base 18: mod_base = um
  modified_base 19: mod_base = OTHER; note = 2'-O-methyladenosine
  modified_base 20: mod_base = cm
  modified_base 21: mod_base = um
  source 1..21: mol_type = other RNA
gtcaacgactctacatttact

SEQ ID NO: 55 - RNA, 21 nt, synthetic construct
  modified_base 1: mod_base = gm
  modified_base 2: mod_base = um
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = cm
  modified_base 7: mod_base = gm
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16: mod_base = um
  modified_base 17: mod_base = um
  modified_base 18: mod_base = um
  modified_base 19: mod_base = OTHER; note = 2'-O-methyladenosine
  modified_base 20: mod_base = cm
  modified_base 21: mod_base = um
  source 1..21: mol_type = other RNA
gtcaacgactctacatttact

SEQ ID NO: 56 - RNA, 21 nt, synthetic construct
  modified_base 1: mod_base = gm
  modified_base 2: mod_base = um
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = cm
  modified_base 7: mod_base = gm
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16: mod_base = um
  modified_base 17: mod_base = um
  modified_base 18: mod_base = um
  modified_base 19: mod_base = OTHER; note = 2'-O-methyladenosine
  modified_base 20: mod_base = cm
  modified_base 21: mod_base = um
  source 1..21: mol_type = other RNA
gtcaacgactctacatttact

SEQ ID NO: 57 - RNA, 21 nt, synthetic construct
  modified_base 1: mod_base = gm
  modified_base 2: mod_base = um
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = cm
  modified_base 7: mod_base = gm
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16: mod_base = um
  modified_base 17: mod_base = um
  modified_base 18: mod_base = um
  modified_base 19: mod_base = OTHER; note = 2'-O-methyladenosine
  modified_base 20: mod_base = cm
  modified_base 21: mod_base = um
  source 1..21: mol_type = other RNA
gtcaacgactctacatttact

SEQ ID NO: 58 - RNA, 21 nt, synthetic construct
  modified_base 1: mod_base = gm
  modified_base 2: mod_base = um
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = cm
  modified_base 7: mod_base = gm
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16: mod_base = um
  modified_base 17: mod_base = um
  modified_base 18: mod_base = um
  modified_base 19: mod_base = OTHER; note = 2'-O-methyladenosine
  modified_base 20: mod_base = cm
  modified_base 21: mod_base = um
  source 1..21: mol_type = other RNA
gtcaacgactctacatttact

SEQ ID NO: 59 - RNA, 21 nt, synthetic construct
  modified_base 1: mod_base = gm
  modified_base 2: mod_base = um
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = cm
  modified_base 7: mod_base = gm
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16: mod_base = um
  modified_base 17: mod_base = um
  modified_base 18: mod_base = um
  modified_base 19: mod_base = OTHER; note = 2'-O-methyladenosine
  modified_base 20: mod_base = cm
  modified_base 21: mod_base = um
  source 1..21: mol_type = other RNA
gtcaacgactctacatttact

SEQ ID NO: 60 - RNA, 21 nt, synthetic construct
  modified_base 1: mod_base = gm
  modified_base 2: mod_base = um
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = cm
  modified_base 7: mod_base = gm
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16: mod_base = um
  modified_base 17: mod_base = um
  modified_base 18: mod_base = um
  modified_base 19: mod_base = OTHER; note = 2'-O-methyladenosine
  modified_base 20: mod_base = cm
  modified_base 21: mod_base = um
  source 1..21: mol_type = other RNA
gtcaacgactctacatttact

SEQ ID NO: 61 - RNA, 21 nt, synthetic construct
  modified_base 1: mod_base = gm
  modified_base 2: mod_base = um
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = cm
  modified_base 7: mod_base = gm
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16: mod_base = um
  modified_base 17: mod_base = um
  modified_base 18: mod_base = um
  modified_base 19: mod_base = OTHER; note = 2'-O-methyladenosine
  modified_base 20: mod_base = cm
  modified_base 21: mod_base = um
  source 1..21: mol_type = other RNA
gtcaacgactctacatttact

SEQ ID NO: 62 - RNA, 21 nt, synthetic construct
  modified_base 1: mod_base = gm
  modified_base 2: mod_base = um
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = cm
  modified_base 7: mod_base = gm
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16: mod_base = um
  modified_base 17: mod_base = um
  modified_base 18: mod_base = um
  modified_base 19: mod_base = OTHER; note = 2'-O-methyladenosine
  modified_base 20: mod_base = cm
  modified_base 21: mod_base = um
  source 1..21: mol_type = other RNA
gtcaacgactctacatttact

SEQ ID NO: 63 - RNA, 21 nt, synthetic construct
  modified_base 1: mod_base = gm
  modified_base 2: mod_base = um
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = cm
  modified_base 7: mod_base = gm
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16: mod_base = um
  modified_base 17: mod_base = um
  modified_base 18: mod_base = um
  modified_base 19: mod_base = OTHER; note = 2'-O-methyladenosine
  modified_base 20: mod_base = cm
  modified_base 21: mod_base = um
  source 1..21: mol_type = other RNA
gtcaacgactctacatttact

SEQ ID NO: 64 - RNA, 21 nt, synthetic construct
  modified_base 1: mod_base = gm
  modified_base 2: mod_base = um
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = cm
  modified_base 7: mod_base = gm
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16: mod_base = um
  modified_base 17: mod_base = um
  modified_base 18: mod_base = um
  modified_base 19: mod_base = OTHER; note = 2'-O-methyladenosine
  modified_base 20: mod_base = cm
  modified_base 21: mod_base = um
  source 1..21: mol_type = other RNA
gtcaacgactctacatttact

SEQ ID NO: 65 - RNA, 21 nt, synthetic construct
  modified_base 1: mod_base = gm
  modified_base 2: mod_base = um
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = cm
  modified_base 7: mod_base = gm
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16: mod_base = um
  modified_base 17: mod_base = um
  modified_base 18: mod_base = um
  modified_base 19: mod_base = OTHER; note = 2'-O-methyladenosine
  modified_base 20: mod_base = cm
  modified_base 21: mod_base = um
  source 1..21: mol_type = other RNA
gtcaacgactctacatttact

SEQ ID NO: 66 - RNA, 21 nt, synthetic construct
  modified_base 1: mod_base = gm
  modified_base 2: mod_base = um
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = cm
  modified_base 7: mod_base = gm
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16: mod_base = um
  modified_base 17: mod_base = um
  modified_base 18: mod_base = um
  modified_base 19: mod_base = OTHER; note = 2'-O-methyladenosine
  modified_base 20: mod_base = cm
  modified_base 21: mod_base = um
  source 1..21: mol_type = other RNA
gtcaacgactctacatttact

SEQ ID NO: 67 - RNA, 21 nt, synthetic construct
  modified_base 1: mod_base = gm
  modified_base 2: mod_base = um
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = cm
  modified_base 7: mod_base = gm
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16: mod_base = um
  modified_base 17: mod_base = um
  modified_base 18: mod_base = um
  modified_base 19: mod_base = OTHER; note = 2'-O-methyladenosine
  modified_base 20: mod_base = cm
  modified_base 21: mod_base = um
  source 1..21: mol_type = other RNA
gtcaacgactctacatttact

SEQ ID NO: 68 - RNA, 21 nt, synthetic construct
  modified_base 1: mod_base = gm
  modified_base 2: mod_base = um
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = cm
  modified_base 7: mod_base = gm
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16: mod_base = um
  modified_base 17: mod_base = um
  modified_base 18: mod_base = um
  modified_base 19: mod_base = OTHER; note = 2'-O-methyladenosine
  modified_base 20: mod_base = cm
  modified_base 21: mod_base = um
  source 1..21: mol_type = other RNA
gtcaacgactctacatttact

SEQ ID NO: 69 - RNA, 21 nt, synthetic construct
  modified_base 1: mod_base = gm
  modified_base 2: mod_base = um
  modified_base 3: mod_base = gm
  modified_base 4: mod_base = OTHER; note = 2'-O-methyladenosine
  modified_base 5: mod_base = OTHER; note = 2'-O-methyladenosine
  modified_base 6: mod_base = gm
  modified_base 7: mod_base = cm
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluoroguanosine
  modified_base 11: mod_base = OTHER; note = 2'-fluoroadenosine
  modified_base 12: mod_base = um
  modified_base 13: mod_base = um
  modified_base 14: mod_base = cm
  modified_base 15: mod_base = um
  modified_base 16: mod_base = OTHER; note = 2'-O-methyladenosine
  modified_base 17: mod_base = um
  modified_base 18: mod_base = cm
  modified_base 19: mod_base = OTHER; note = 2'-O-methyladenosine
  modified_base 20: mod_base = um
  modified_base 21: mod_base = OTHER; note = 2'-O-methyladenosine
  source 1..21: mol_type = other RNA
gtgaagcacgattctatcata

SEQ ID NO: 70 - RNA, 21 nt, synthetic construct
  modified_base 1: mod_base = gm
  modified_base 2: mod_base = OTHER; note = 2'-O-methyladenosine
  modified_base 3: mod_base = gm
  modified_base 4: mod_base = OTHER; note = 2'-O-methyladenosine
  modified_base 5: mod_base = cm
  modified_base 6: mod_base = OTHER; note = 2'-O-methyladenosine
  modified_base 7: mod_base = um
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorocytidine
  modified_base 11: mod_base = OTHER; note = 2'-fluorocytidine
  modified_base 12: mod_base = cm
  modified_base 13: mod_base = OTHER; note = 2'-O-methyladenosine
  modified_base 14: mod_base = gm
  modified_base 15: mod_base = OTHER; note = 2'-O-methyladenosine
  modified_base 16: mod_base = OTHER; note = 2'-O-methyladenosine
  modified_base 17: mod_base = um
  modified_base 18: mod_base = cm
  modified_base 19: mod_base = cm
  modified_base 20: mod_base = um
  modified_base 21: mod_base = OTHER; note = 2'-O-methyladenosine
  source 1..21: mol_type = other RNA
gagacataccccagaatccta

SEQ ID NO: 71 - RNA, 21 nt, synthetic construct
  modified_base 1: mod_base = OTHER; note = 2'-O-methyladenosine
  modified_base 2: mod_base = gm
  modified_base 3: mod_base = cm
  modified_base 4: mod_base = gm
  modified_base 5: mod_base = OTHER; note = 2'-O-methyladenosine
  modified_base 6: mod_base = gm
  modified_base 7: mod_base = cm
  modified_base 8: mod_base = um
  modified_base 9: mod_base = OTHER; note = 2'-fluoroguanosine
  modified_base 10: mod_base = OTHER; note = 2'-fluoroadenosine
  modified_base 11: mod_base = OTHER; note = 2'-fluorocytidine
  modified_base 12: mod_base = OTHER; note = 2'-O-methyladenosine
  modified_base 13: mod_base = um
  modified_base 14: mod_base = cm
  modified_base 15: mod_base = um
  modified_base 16: mod_base = OTHER; note = 2'-O-methyladenosine
  modified_base 17: mod_base = um
  modified_base 18: mod_base = um
  modified_base 19: mod_base = cm
  modified_base 20: mod_base = gm
  modified_base 21: mod_base = OTHER; note = 2'-O-methyladenosine
  source 1..21: mol_type = other RNA
agcgagctgacatctattcga

SEQ ID NO: 72 - RNA, 21 nt, synthetic construct
  modified_base 1: mod_base = gm
  modified_base 2: mod_base = um
  modified_base 3: mod_base = gm
  modified_base 4: mod_base = OTHER; note = 2'-O-methyladenosine
  modified_base 5: mod_base = OTHER; note = 2'-O-methyladenosine
  modified_base 6: mod_base = gm
  modified_base 7: mod_base = cm
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluoroguanosine
  modified_base 11: mod_base = OTHER; note = 2'-fluoroadenosine
  modified_base 12: mod_base = um
  modified_base 13: mod_base = um
  modified_base 14: mod_base = cm
  modified_base 15: mod_base = um
  modified_base 16: mod_base = OTHER; note = 2'-O-methyladenosine
  modified_base 17: mod_base = um
  modified_base 18: mod_base = cm
  modified_base 19: mod_base = OTHER; note = 2'-O-methyladenosine
  modified_base 20: mod_base = um
  modified_base 21: mod_base = OTHER; note = 2'-O-methyladenosine
  source 1..21: mol_type = other RNA
gtgaagcacgattctatcata

SEQ ID NO: 73 - RNA, 21 nt, synthetic construct
  modified_base 1: mod_base = cm
  modified_base 2: mod_base = cm
  modified_base 3: mod_base = um
  modified_base 4: mod_base = gm
  modified_base 5: mod_base = OTHER; note = 2'-O-methyladenosine
  modified_base 6: mod_base = OTHER; note = 2'-O-methyladenosine
  modified_base 7: mod_base = OTHER; note = 2'-O-methyladenosine
  modified_base 8: mod_base = um
  modified_base 9: mod_base = OTHER; note = 2'-fluoroguanosine
  modified_base 10: mod_base = OTHER; note = 2'-fluorocytidine
  modified_base 11: mod_base = OTHER; note = 2'-fluorouridine
  modified_base 12: mod_base = um
  modified_base 13: mod_base = gm
  modified_base 14: mod_base = OTHER; note = 2'-O-methyladenosine
  modified_base 15: mod_base = um
  modified_base 16: mod_base = gm
  modified_base 17: mod_base = OTHER; note = 2'-O-methyladenosine
  modified_base 18: mod_base = um
  modified_base 19: mod_base = OTHER; note = 2'-O-methyladenosine
  modified_base 20: mod_base = cm
  modified_base 21: mod_base = OTHER; note = 2'-O-methyladenosine
  source 1..21: mol_type = other RNA
cctgaaatgcttgatgataca

SEQ ID NO: 74 - RNA, 21 nt, synthetic construct
  modified_base 1: mod_base = gm
  modified_base 2: mod_base = OTHER; note = 2'-O-methyl-2-aminoadenosine
  modified_base 3: mod_base = OTHER; note = 2'-O-methyladenosine
  modified_base 4: mod_base = OTHER; note = 2'-O-methyladenosine
  modified_base 5: mod_base = um
  modified_base 6: mod_base = gm
  modified_base 7: mod_base = cm
  modified_base 8: mod_base = um
  modified_base 9: mod_base = OTHER; note = 2'-fluorouridine
  modified_base 10: mod_base = OTHER; note = 2'-fluoroguanosine
  modified_base 11: mod_base = OTHER; note = 2'-fluoroadenosine
  modified_base 12: mod_base = um
  modified_base 13: mod_base = gm
  modified_base 14: mod_base = OTHER; note = 2'-O-methyladenosine
  modified_base 15: mod_base = um
  modified_base 16: mod_base = OTHER; note = 2'-O-methyladenosine
  modified_base 17: mod_base = cm
  modified_base 18: mod_base = OTHER; note = 2'-O-methyladenosine
  modified_base 19: mod_base = OTHER; note = 2'-O-methyl-2-aminoadenosine
  modified_base 20: mod_base = um
  modified_base 21: mod_base = OTHER; note = 2'-O-methyladenosine
  source 1..21: mol_type = other RNA
gnaatgcttgatgatacanta

SEQ ID NO: 75 - RNA, 21 nt, synthetic construct
  modified_base 1: mod_base = gm
  modified_base 2: mod_base = um
  modified_base 3: mod_base = cm
  modified_base 4: mod_base = um
  modified_base 5: mod_base = gm
  modified_base 6: mod_base = um
  modified_base 7: mod_base = cm
  modified_base 8: mod_base = OTHER; note = 2'-O-methyladenosine
  modified_base 9: mod_base = OTHER; note = 2'-fluorouridine
  modified_base 10: mod_base = OTHER; note = 2'-fluoroadenosine
  modified_base 11: mod_base = OTHER; note = 2'-fluoroguanosine
  modified_base 12: mod_base = um
  modified_base 13: mod_base = um
  modified_base 14: mod_base = cm
  modified_base 15: mod_base = cm
  modified_base 16: mod_base = OTHER; note = 2'-O-methyladenosine
  modified_base 17: mod_base = OTHER; note = 2'-O-methyladenosine
  modified_base 18: mod_base = cm
  modified_base 19: mod_base = OTHER; note = 2'-O-methyladenosine
  modified_base 20: mod_base = OTHER; note = 2'-O-methyladenosine
  modified_base 21: mod_base = um
  source 1..21: mol_type = other RNA
gtctgtcatagttccaacaat

SEQ ID NO: 76 - RNA, 21 nt, synthetic construct
  modified_base 1: mod_base = gm
  modified_base 2: mod_base = cm
  modified_base 3: mod_base = um
  modified_base 4: mod_base = gm
  modified_base 5: mod_base = gm
  modified_base 6: mod_base = um
  modified_base 7: mod_base = OTHER; note = 2'-O-methyladenosine
  modified_base 8: mod_base = um
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluoroguanosine
  modified_base 12: mod_base = OTHER; note = 2'-O-methyladenosine
  modified_base 13: mod_base = OTHER; note = 2'-O-methyladenosine
  modified_base 14: mod_base = um
  modified_base 15: mod_base = OTHER; note = 2'-O-methyladenosine
  modified_base 16: mod_base = um
  modified_base 17: mod_base = cm
  modified_base 18: mod_base = OTHER; note = 2'-O-methyladenosine
  modified_base 19: mod_base = um
  modified_base 20: mod_base = gm
  modified_base 21: mod_base = OTHER; note = 2'-O-methyladenosine
  source 1..21: mol_type = other RNA
gctggtatctgaatatcatga